(19) United States
(12) Patent Application Publication
(10) Pub. No.: US 2024/0252677 A1
(43) Pub. Date: Aug. 1, 2024

(54) ONE-COMPONENT DELIVERY SYSTEM FOR NUCLEIC ACIDS

(71) Applicant: The Trustees of the University of Pennsylvania, Philadelphia, PA (US)

(72) Inventors: Virgil Percec, Philadelphia, PA (US); Drew Weissman, Philadelphia, PA (US); Dapeng Zhang, Philadelphia, PA (US); Elena Atochina-Vasserman, Huntingdon Valley, PA (US); Devendra Maurya, Philadelphia, PA (US); Qi Xiao, Philadelphia, PA (US)

(21) Appl. No.: 18/563,503

(22) PCT Filed: May 24, 2022
(86) PCT No.: PCT/US22/30694

§ 371 Date: Nov. 22, 2023

Related U.S. Application Data
(60) Provisional application No. 63/316,794, filed on Mar. 4, 2022.
(60) Provisional application No. 63/253,348, filed on Oct. 7, 2021.
(60) Provisional application No. 63/192,236, filed on May 24, 2021.

(51) Int. Cl. A61K 48/00; A61K 9/51; A61K 39/385; A61K 47/54

(52) U.S. Cl. CPC A61K 48/0033; A61K 9/5123; A61K 39/385; A61K 47/542; A61K 47/545

(57) ABSTRACT

The invention relates to amphiphilic Janus dendrimers which may form nanoparticles. The invention also relates to methods of inducing an adaptive immune response in a subject comprising administering to the subject an effective amount of a composition comprising at least one nucleoside-modified RNA encoding at least one antigen and at least one amphiphilic Janus dendrimer and to methods of delivering an agent to a subject in need thereof, said method comprising the step of delivering to the subject a composition comprising an agent encapsulated by a nanoparticle.

### CROSS REFERENCE TO RELATED APPLICATIONS

This application claims priority to and the benefit of U.S. Provisional Application No. 63/192,236, filed on May 24, 2021, U.S. Provisional Application No. 63/253,348, filed on Oct. 7, 2021, and U.S. Provisional Application No. 63/316,794, filed on Mar. 4, 2022, the contents of which are incorporated hereby by reference herein in their entireties.

### STATEMENT REGARDING FEDERALLY SPONSORED RESEARCH OR DEVELOPMENT

This invention was made with government support under 1720530, 1807127, 2104554 awarded by the National Science Foundation and TR002776 awarded by the National Institutes of Health. The government has certain rights in the invention.

### BACKGROUND OF THE INVENTION

Delivery of exogenously produced nucleic acids into cells and/or their nucleus to modify protein expression by viral and nonviral vectors represents one of the most fundamental concepts of nanomedicine. Both viral and nonviral delivery systems exhibit advantages and disadvantages. Viral vectors have high transfection efficiency (95%) and higher specificity for cell targeting including for unnatural cells. Some drawbacks of viral gene delivery include immunogenicity, cytotoxicity, difficulty of their assembly, inflammatory responses to repeated administration and the potential for insertional mutagenesis. Nonviral delivery is biosafe, exhibits lower toxicity and lower immunogenicity but it is less transfection efficient (1-2%) and the vectors are less stable than viral vectors. Covalent and supramolecular dendrimers complexed on their cationic periphery groups with the nucleic acid have been employed as nonviral vectors for cell transfection of DNA.

Four component lipid nanoparticles (LNPs) containing ionizable lipids, phospholipids, cholesterol for improved mechanical properties and a poly(ethylene glycol) (PEG)-conjugated lipid that provides stability, represents the current leading nonviral vector for the delivery of mRNA. Shortcomings of LNPs production and stability are demonstrated by a microfluidic device required for their assembly and the need to be stored at extremely low temperatures (−70° C.). Their design, synthesis and assembly was inspired by stealth liposomes developed to deliver low molar mass drugs.

Since RNA is less stable than DNA, it must be protected by encapsulation before being released in the cell. At acidic pH (pH 3 to 5), LNPs can encapsulate large quantities of mRNA when the pKₐ of the ionizable amine is less than 7. At physiological pH (7.4) LNPs have a nearly neutral surface charge and a high positive charge at the endosomal pH. In endosomal membranes the electrostatic interaction between the catatonically charged LNPs and the naturally occurring anionic lipids has been suggested to be responsible for the release of the RNA. One of the major limitations of the four-component vector is the unknown distribution of its four components in the LNP. The segregation of the neutral ionizable lipid as an oil-phase in the core of the LNPs is considered to be responsible for their very low transfection efficiency (1-2%). The second deficiency of the LNP is provided by the PEG-conjugated lipid and is known as the “PEG dilemma”. PEG conjugated to LNP increases the circulation time in blood after intravenous injection. However, the same PEG is known to decrease gene expression with up to four-order of magnitude by decreasing intracellular trafficking of cellular uptake and endosomal escape.

Charge-altering releasable transporters (CARTS) have also been demonstrated for the delivery of mRNA. This delivery concept is unrelated to the viral and nonviral LNP based methodologies discussed above. Artificial and synthetic vesicles, such as liposomes and polymersomes have been elaborated both for drug delivery and also as mimics of natural cells. Dendrimersomes (DSs), which are assembled from amphiphilic Janus dendrimers (JDs), have been shown to exchibit excellent mechanical properties and stability including in serum. Amphiphilic JDs with sugars conjugated on their hydrophilic part, denoted Janus glycodendrimers (JGDs), self-assemble into glycodendrimersomes (GDSs), which mimic the glycans of biological membranes and bind sugar binding proteins. Both JDs and JGDs self-assemble into monodisperse DSs and GDSs with unilamellar or multilamellar structures by simple injection rather than by the microfluidic technology and their dimensions can be predicted. Sequence-defined JGDs self-assemble by injection into GDSs. They demonstrated that a lower sugar density in a defined sequence elicited higher bioactivity to sugar-binding proteins.

Thus, there is a need in the art for compositions and methods for the delivery of mRNA. The present invention satisfies this unmet need.

### SUMMARY OF THE INVENTION

In one aspect, the present invention relates, in part, to an ionizable amphiphilic Janus dendrimer having the structure of Formula (I):

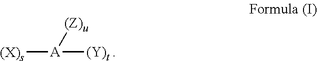

In some embodiments, A is a polyvalent group comprising at least one selected from

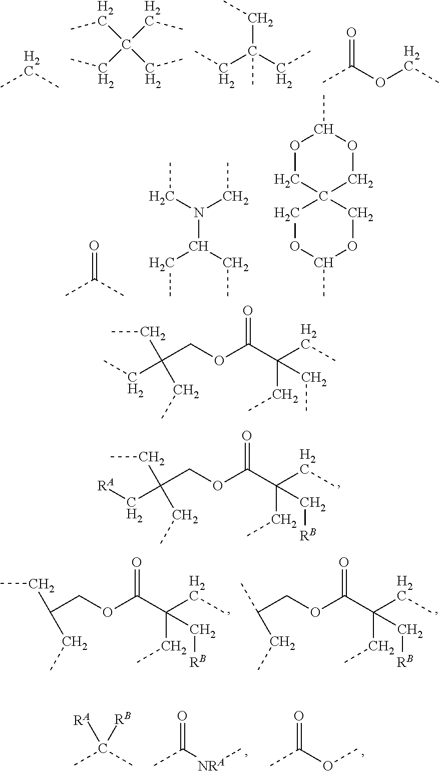

or any combination thereof. In some embodiments A is a polyvalent group comprising at least one selected from

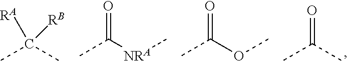

or any combination thereof.

In some embodiments, dashed lines represent a binding site of one of X, Y, or Z.

In some embodiments, X is a hydrophilic group comprising at least one amine.

In some embodiments, Y is a lipophilic group comprising at least one C₁-C₃₀-alkyl chain. In some embodiments, Y is a lipophilic group comprising at least two C₁-C₃₀-alkyl chains having differing numbers of carbon atoms

In some embodiments, Z comprises at least one selected from ethylene glycol, diethylene glycol, triethylene glycol, or polyethylene glycol chain.

In some embodiments, R^{A} and R^{B} are independently selected from hydrogen, halide, hydroxy, C₁-C₃₀-alkyl, C₁-C₃₀-alkyl halide, C₁-C₃₀-alkoxy, C₁-C₃₀-alkoxy halide, or any combination thereof.

In some embodiments, s is an integer from 0 to 5. In some embodiments, s is an integer from 1 to 5.

In some embodiments, t is an integer from 0 to 5. In some embodiments, t is an integer from 1 to 5.

In some embodiments, u is an integer from 0 to 4.

In some embodiments, the sum of s, t, and u is equal to the valency of A.

For example, in some embodiments, A is represented by

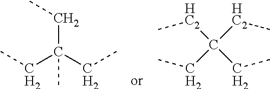

and s and t are each 2.

In some embodiments, A is represented by

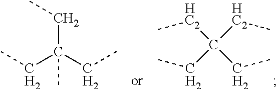

t is 2; s is 1; and u is 0 or 1.

In some embodiments, A is represented by

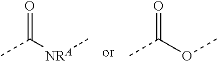

and s and t are each 1.

In some embodiments, the ionizable amphiphilic Janus dendrimer having the structure of Formula (I) is an ionizable amphiphilic Janus dendrimer having the structure of Formula (II):

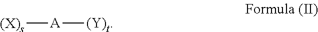

In some embodiments, each occurrence of X is independently selected from

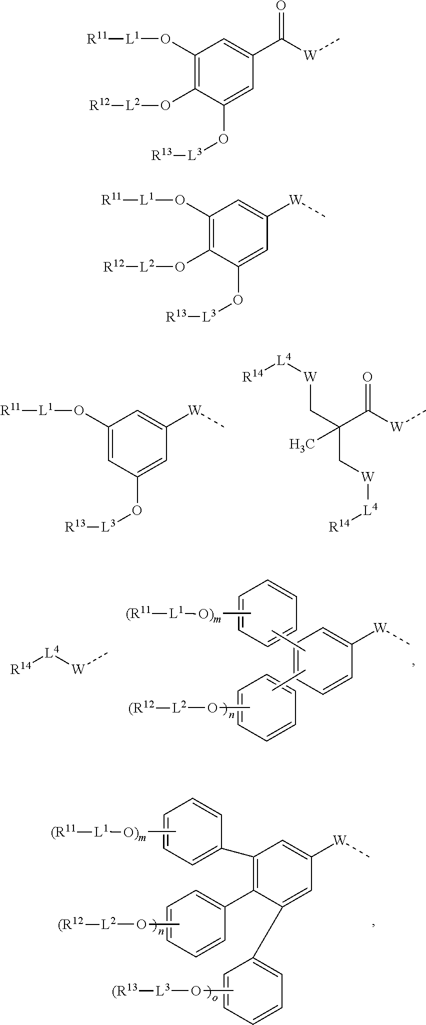

or any combination thereof.

In some embodiments, dashed lines indicate the connection to A.

In some embodiments, each occurrence of m, n, o is independently an integer from 1 to 20. In some embodiments, each occurrence of m, n, o is independently an integer from 1 to 10. In some embodiments, each occurrence of m, n, o is independently an integer from 1 to 5.

In some embodiments, each occurrence of W is independently selected from C═O, C(R^{W})(R^{W}), NR^{W}, O, or S. In some embodiments, each occurrence of R^{W} is independently selected from hydrogen, halide, hydroxy, alkyl, alkyl halide, aryl, aryl halide, alkoxy, alkoxy halide, or any combination thereof.

In some embodiments, each occurrence of L¹, L², L³, and L⁴ is independently a covalent bond or a divalent linking group selected from alkylene, cycloalkylene, heteroalkylene, heterocycloalkylene, alkenylene, alkynylene, arylene, heteroarylene, silyl, amine, amide, ester, ether, carbonyl, carbamate, thioether, thioester, disulfide, hydrazine, urea, thiourea, phosphate, poly(alkyl ether), heteroatom, or any combinations thereof.

In some embodiments, each occurrence of R¹¹, R¹², R¹³, and R¹⁴ is independently selected from hydrogen, deuterium, alkyl, aryl, heteroaryl, cycloalkyl, alkoxy, phenoxy, amine, heterocycloalkyl, carbonyl, or any combinations thereof. In some embodiments, at least one occurrence of R¹¹, R¹², R¹³, or R¹⁴ is —C(O)(CH₂)ₘN(R¹)(R²). In some embodiments, each occurrence of m is independently an integer from 1 to 10. In some embodiments, each occurrence of R¹ and R² is independently selected from hydrogen, deuterium, alkyl, aryl, cycloalkyl, amine, heterocycloalkyl, carbonyl, or any combinations thereof. In some embodiments, R¹ and R² may together form a ring. In some embodiments, each occurrence of R¹¹, R¹², R¹³, and R¹⁴ is independently selected from

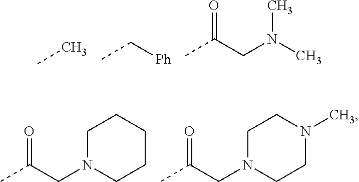

or any combination thereof.

In some embodiments, X comprises at least two tertiary amines.

In some embodiments, each occurrence of X is independently selected from

or any combination thereof.

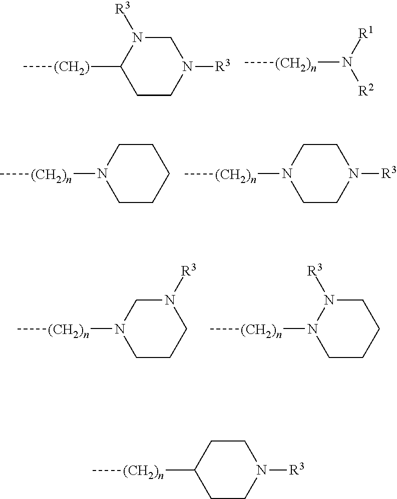

In some embodiments, each occurrence of R³ is independently selected from hydrogen, (CH₂)ₙ, (CH₂)ₙ—OH, or any combination thereof.

In some embodiments, each occurrence of X is independently selected from

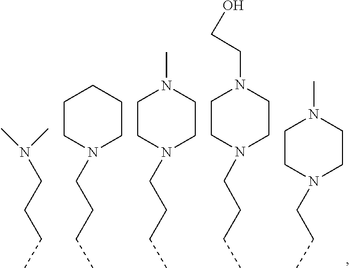

or any combination thereof.

In some embodiments, each occurrence of Y is independently selected from

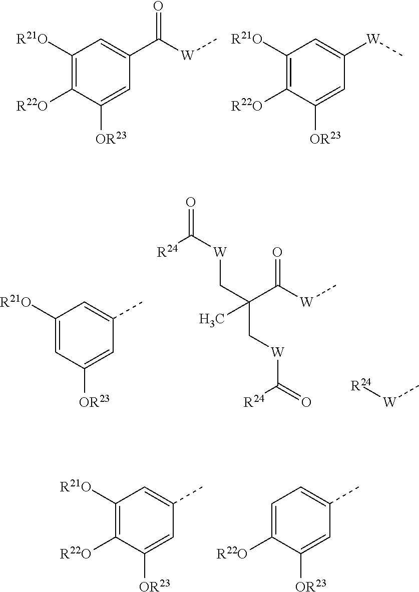

or any combination thereof.

In some embodiments, each occurrence of R²¹, R²², R²³, and R²⁴ independently comprises a linear or branched C₁-C₅₀-alkyl. In some embodiments, each occurrence of R²¹, R²², R²³, and R²⁴ independently comprises C₁-C₃₀-alkyl.

In some embodiments, each occurrence of Y is independently selected from

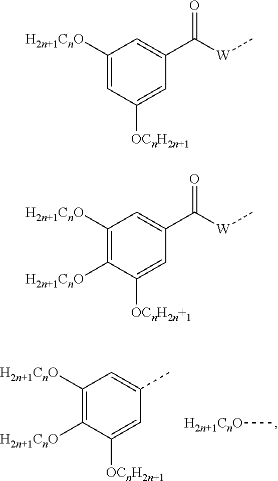

or any combination thereof. In some embodiments, n is an integer from 1 to 30.

In some embodiments, each occurrence of Y is independently selected from

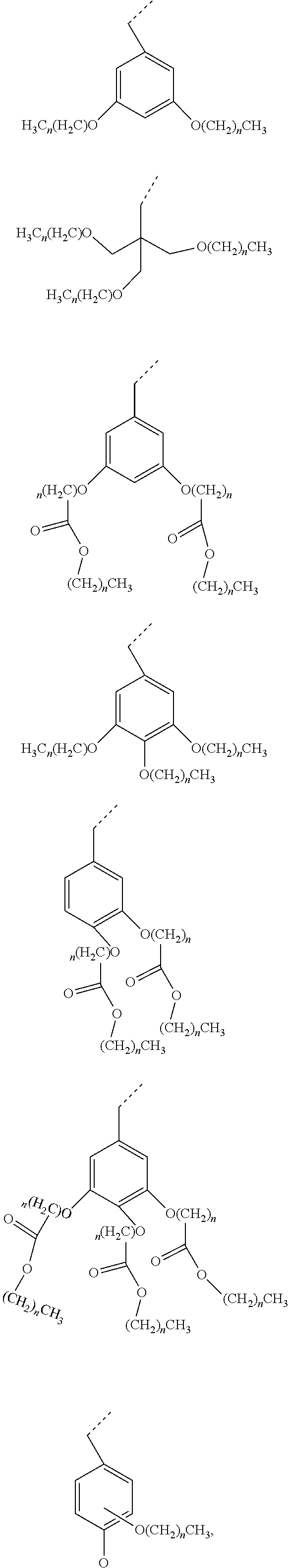

or any combination thereof.

In some embodiments, each occurrence of —(CH₂)ₙCH₃, —O(CH₂)ₙ—, and —O(CH₂)ₙCH₃ is independently linear or branched.

In some embodiments, the ionizable amphiphilic Janus dendrimer comprises a first Y and a second Y. In some embodiments, the first Y comprises an alkyl chain having an even number of carbon atoms and the second Y comprises an alkyl chain having an odd number of carbon atoms. In some embodiments, the ratio between the carbon atoms in the first Y and the carbon atoms in the second Y is greater than or equal to 3 and less than 7.

In some embodiments, u is 1 or 2.

In some embodiments, each occurrence of Z is independently selected from

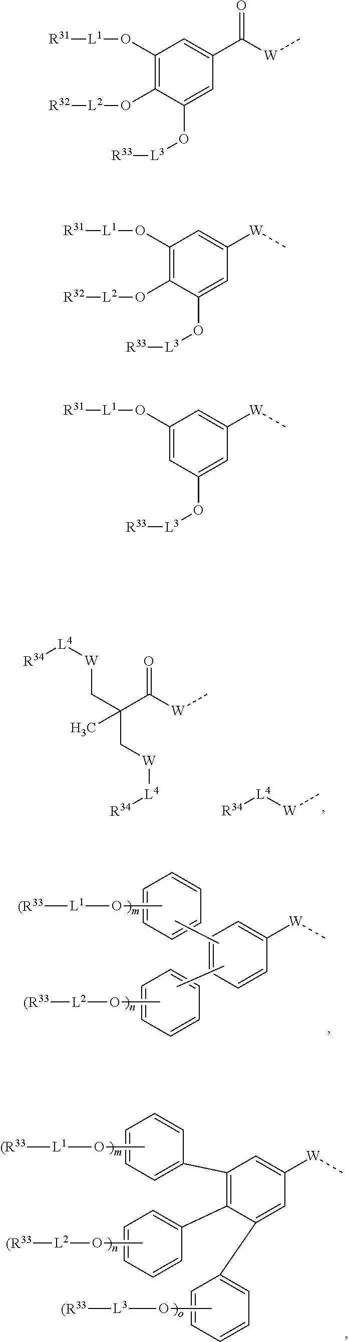

or any combination thereof.

In some embodiments, each occurrence of R³¹, R³², R³³, and R³⁴ is independently selected from hydrogen, deuterium, alkyl, aryl, heteroaryl, cycloalkyl, alkoxy, phenoxy, amine, heterocycloalkyl, carbonyl, or any combinations thereof. In some embodiments, each occurrence of Z is independently selected from

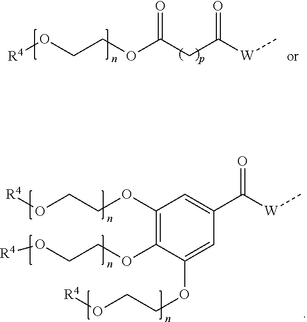

In some embodiments, each occurrence of n is independently an integer from 1 to 100. In some embodiments, each occurrence of p is independently an integer from 1 to 10. In some embodiments, each occurrence of R⁴ is independently selected from hydrogen, deuterium, alkyl, aryl, or any combinations thereof.

In some embodiments, the ionizable amphiphilic Janus dendrimer comprises a homochiral, racemic, or achiral branding points.

In some embodiments, the ionizable amphiphilic Janus dendrimer is a homochiral ionizable amphiphilic Janus dendrimer, racemic ionizable amphiphilic Janus dendrimer, or achiral ionizable amphiphilic Janus dendrimer.

In some embodiments, the ionizable amphiphilic Janus dendrimer is an ionizable amphiphilic Janus dendrimer having a structure selected from the group consisting of at least one structure of FIG. 12, at least one structure of FIG. 13, at least one structure of FIG. 14, at least one structure of FIG. 47, at least one structure of FIG. 60, at least one structure of FIG. 80, and any combination thereof.

In one aspect, the present invention relates, in part, to a nanoparticle comprising at least one ionizable amphiphilic Janus dendrimer of the present invention.

In some embodiments, the nanoparticle comprises a first ionizable amphiphilic Janus dendrimer and a second ionizable amphiphilic Janus dendrimer. In some embodiments, the first ionizable amphiphilic Janus dendrimer has a different structure than the second ionizable amphiphilic Janus dendrimer.

In some embodiments, the nanoparticle comprises a homochiral ionizable amphiphilic Janus dendrimer, achiral ionizable amphiphilic Janus dendrimer, or any combination thereof.

In some embodiments, the nanoparticle is a unilamellar nanoparticle or an onion multilamellar nanoparticle. In some embodiments, the nanoparticle comprises a racemic ionizable amphiphilic Janus dendrimer. In some embodiments, the nanoparticle is a multilamellar nanoparticle.

In some embodiments, the nanoparticle further comprises at least one agent. In some embodiments, the at least one agent comprises a diagnostic agent, detectable agent, therapeutic agent, nucleic acid molecule, or any combination thereof. In some embodiments, the at least one agent is selected from an mRNA, siRNA, microRNA, CRISPR-Cas9, sgRNA, small molecule, protein, antibody, peptide, protein, or any combination thereof.

In some embodiments, the at least one agent comprises a nucleic acid molecule. In some embodiments, the nucleic acid molecule is a DNA molecule or an RNA molecule. In some embodiments, the nucleic acid molecule is selected from cDNA, cRNA, CirRNA, mRNA, miRNA, siRNA, sgRNA, modified RNA, tRNA, antagomir, antisense molecule, targeted nucleic acid, or any combination thereof. In some embodiments, the nucleic acid molecule encodes at least one selected from an antigen, antibody, gene editing molecule, chimeric antigen receptor (CAR), or any combination thereof. In one embodiment, the nucleoside-modified RNA comprises pseudouridine. In one embodiment, the nucleoside-modified RNA comprises pseudouridine plus 5-methyl-cytosine. In one embodiment, the nucleoside-modified RNA comprises 5-methyl-uridine. In one embodiment, the nucleoside-modified RNA comprises 1-methyl-pseudouridine.

In one aspect, the invention relates to a composition comprising at least one dendrimersome nanoparticle as described herein.

In one aspect, the present invention relates, in part, a composition comprising at least one ionizable amphiphilic Janus dendrimer of the present invention and/or at least one nanoparticle of the present invention.

In one embodiment, the composition further comprises an adjuvant.

In one embodiment, the composition is a pharmaceutical composition.

In one embodiment, the composition is a vaccine.

In one aspect, the present invention relates, in part, a method of delivering an agent to a subject in need thereof using at least one nanoparticle or a composition of the present invention comprising the same.

In some embodiment, the method further comprises delivering the agent to the liver of the subject.

In some embodiment, the method further comprises delivering the agent to the spleen of the subject.

In some embodiment, the method further comprises delivering the agent to the lungs of the subject.

In some embodiment, the method treats or prevents at least one condition selected from a viral infection, bacterial infection, fungal infection, parasitic infection, cancer, disease or disorder associated with cancer, autoimmune disease or disorder, or any combination thereof.

In some embodiments, the agent is encapsulated within the nanoparticle. In one embodiment, the agent is any agent described herein. For example, in one embodiment, the agent is a composition for protein replacement therapy. In one embodiment, the agent is a composition for gene editing. In one embodiment, the agent is a vaccine.

In one aspect, the present invention relates, in part, a method of preventing or treating a disease or disorder in a subject in need thereof using at least one nanoparticle or a composition of the present invention comprising the same. In some embodiments, the disease or disorder is selected from a viral infection, bacterial infection, fungal infection, parasitic infection, cancer, disease or disorder associated with cancer, autoimmune disease or disorder, or any combination thereof.

In one aspect, the present invention relates, in part, a method of inducing an immune response in a subject in need thereof using at least one nanoparticle or a composition of the present invention comprising the same.

In one aspect, the invention relates to a method of delivering an agent to a subject in need thereof, said method comprising administering at least one dendrimersome nanoparticle described herein or a composition comprising the same to a subject.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a schematic depicting an exemplary one-component nanoparticle (DNP) for mRNA delivery.

FIG. 2 depicts a schematic depicting a four-component lipid nanoparticle (LNP) system for mRNA delivery.

FIG. 3 depicts a schematic representation of hydrophilic acids, and hydrophobic acids, and linkers employed in the exemplary DNPs.

FIG. 4 depicts a schematic representation of the six libraries containing 52 ionizable amphiphilic Janus dendrimers (IAJDs).

FIG. 5 depicts representative Luciferase expression in HEK293T cells with DNPs encapsulating Luciferase-mRNA.

FIG. 6 depicts representative assay results of in vivo transfection results of one-component DNPs. Diameters and polydispersities of DNPs (both in black) and pKₐ values of IAJDs (in blue) are shown under the number of the IAJD molecule. All these data are printed on top of each mice image. The luminescence values are also shown.

FIG. 7 depicts representative results quantifying the of luciferase signal from in vivo images.

FIG. 8 depicts representative results comparing concentration and sequence of IAs of IAJDs on activity in vitro and in vivo. At right are the schematic representations of the IAJDs employed.

FIG. 9 depicts representative in vivo images of organs.

FIG. 10 depicts representative images of mRNA delivery to different organs by one-component DNPs.

FIG. 11 depicts representative results demonstrating examples of excellent stability of DNPs assembled from IAJD9, IAJD22, IAJD33, IAJD34, IAJD32, IAJD33+IAJD32 (2%), IAJD46, and IAJD47.

FIG. 12 depicts exemplary compounds from Single-Single IAJD Libraries 1-4. Deep-yellow color means that the molecule shows activity both in vitro and in vivo except IAJD19, 23 and 45, which only show activity in vivo but no activity in vitro. Light-yellow color means that the molecule only shows activity in vitro. White color means that the molecule shows no activity neither in vitro nor in vivo.

FIG. 13 depicts exemplary compounds from Twin-Twin IAJD Library 5. Deep-yellow color means that the molecule shows activity both in vitro and in vivo. Light-yellow color means that the molecule only shows activity in vitro. White color means that the molecule shows no activity neither in vitro nor in vivo.

FIG. 14 depicts exemplary compounds from Hybrid Twin-Mixed IAJD Library 6. Deep-yellow color means that the molecule shows activity both in vitro and in vivo. Light-yellow color means that the molecule only shows activity in vitro. White color means that the molecule shows no activity neither in vitro nor in vivo.

FIG. 15 depicts representative MALDI-TOF MS spectra of compound 91c (4/2_{DMBA}^{1,3}_{Bn}²PEG⁴). At right is the zoomed spectrum.

FIG. 16 depicts results demonstrating the effect of vortex time on the dimensions of assemblies of exemplary dendrimer IAJD9 (4.0 mg/mL in tris buffer).

FIG. 17 depicts representative DLS data of assemblies of IAJD9 (8.00 to 1.00 mg/ml) in tris buffer (pH=7.4).

FIG. 18 depicts representative DLS data of assemblies of IAJD22 (8.0 to 1.0 mg/ml) in tris buffer (pH=7.4).

FIG. 19 depicts representative DLS data of assemblies of IAJD27 (8.0 to 1.0 mg/ml) in tris buffer (pH=7.4).

FIG. 20 depicts representative DLS data of DNPs assembled from IAJD1 and IAJD2.

FIG. 21 depicts representative DLS data of DNPs assembled from IAJD8 and IAJD9.

FIG. 22 depicts representative DLS data of DNPs assembled from IAJD10 and IAJD17.

FIG. 23 depicts representative DLS data of DNPs assembled from IAJD18 and IAJD19.

FIG. 24 depicts representative DLS data of DNPs assembled from IAJD20 and IAJD21.

FIG. 25 depicts representative DLS data of DNPs assembled from IAJD22 and IAJD23.

FIG. 26 depicts representative DLS data of DNPs assembled from IAJD24 and IAJD25.

FIG. 27 depicts representative DLS data of DNPs assembled from IAJD26 and IAJD27.

FIG. 28 depicts representative DLS data of DNPs assembled from IAJD28 and IAJD29.

FIG. 29 depicts representative DLS data of DNPs assembled from IAJD30 and IAJD31.

FIG. 30 depicts representative DLS data of DNPs assembled from IAJD33 and IAJD34.

FIG. 31 depicts representative DLS data of DNPs assembled from IAJD37 and IAJD40.

FIG. 32 depicts representative DLS data of DNPs assembled from IAJD43 and IAJD44.

FIG. 33 depicts representative DLS data of DNPs assembled from IAJD45 and IAJD46.

FIG. 34 depicts representative DLS data of DNPs assembled from IAJD47.

FIG. 35 depicts representative results showing examples of good stability of DNPs assembled from IAJD27 and not good stability of DNPs assembled from IAJD30 and IAJD31.

FIG. 36 depicts representative results showing dimensions of DNPs assembled from IAJD33+IAJD32 (2%) in 1% fetal bovine serum.

FIG. 37 depicts representative titration curves showing changes in solution pH in response to addition of a strong acid for IAJD molecules.

FIG. 38 depicts representative titration curves showing changes in solution pH in response to addition of a strong acid for IAJD molecules.

FIG. 39 depicts representative titration curves showing changes in solution pH in response to addition of a strong acid for IAJD molecules.

FIG. 40 depicts representative titration curves showing changes in solution pH in response to addition of a strong acid for IAJD molecules.

FIG. 41 depicts representative titration curves showing changes in solution pH in response to addition of a strong acid for IAJD molecules.

FIG. 42 depicts representative titration curves showing changes in solution pH in response to addition of a strong acid for IAJD molecules.

FIG. 43 depicts representative titration curves showing changes in solution pH in response to addition of a strong acid for IAJD molecules.

FIG. 44 depicts representative titration curves showing changes in solution pH in response to addition of a strong acid for IAJD molecules.

FIG. 45 depicts representative titration curves showing changes in solution pH in response to addition of a strong acid for IAJD molecules.

FIG. 46 depicts representative titration curves showing changes in solution pH in response to addition of a strong acid for IAJD molecules.

FIG. 47 depicts schematic representation of additional compounds of the present invention as well as the pKₐ values determined for these compounds.

FIG. 48 depicts representative results demonstrating the distribution of nanoparticles in mice as a function of the IAJD.

FIG. 49 depicts representative results demonstrating a comparison of in vivo and in vitro luminescence for different IAJDs.

FIG. 50 depicts representative DLS data of DNPs assembled from IAJD64, IAJD65, IAJD66, IAJD70, IAJD71, IAJD74, IAJD75, IAJD76, and IAJD77.

FIG. 51 depicts representative DLS data of DNPs assembled from IAJD78, IAJD79, IAJD81, IAJD82, IAJD83, IAJD84, IAJD85, IAJD86, and IAJD87.

FIG. 52 depicts representative DLS data of DNPs assembled from IAJD88, IAJD89, IAJD91, IAJD95, IAJD96, IAJD97, IAJD98, IAJD99, and IAJD103.

FIG. 53 depicts representative DLS data of DNPs assembled from IAJD105, IAJD106, IAJD107, and IAJD108.

FIG. 54 depicts representative titration curves showing changes in solution pH in response to addition of a strong acid for IAJD molecules.

FIG. 55 depicts representative titration curves showing changes in solution pH in response to addition of a strong acid for IAJD molecules.

FIG. 56 depicts representative titration curves showing changes in solution pH in response to addition of a strong acid for IAJD molecules.

FIG. 57 depicts representative titration curves showing changes in solution pH in response to addition of a strong acid for IAJD molecules.

FIG. 58 depicts representative results demonstrating a comparison of in vivo and in vitro efficacy.

FIG. 59 depicts a schematic representation of a synthesis of nonsymmetric IAJDs.

FIG. 60 depicts schematic representations of IAJD 81 through IAJD 159

FIG. 61 depicts representative pKₐ and luminescence of selected IAJDs.

FIG. 62 depicts representative results demonstrating the luminescence of selected IAJDs by location in mice.

FIG. 63 depicts representative DLS data of DNPs assembled from IAJD113 through IAJD120 and IAJD122.

FIG. 64 depicts representative DLS data of DNPs assembled from IAJD124 through IAJD130, IAJD133, IAJD135, and IAJD136.

FIG. 65 depicts representative DLS data of DNPs assembled from IAJD138 and IAJD141 through IAJD148.

FIG. 66 depicts representative DLS data of DNPs assembled from IAJD149 through IAJD154, IAJD161, IAJD162, and IAJD171.

FIG. 67 depicts representative DLS data of DNPs assembled from IAJD172, IAJD173, IAJD177, IAJD178, IAJD110, IAJD111, and IAJD155 through IAJD159.

FIG. 68 depicts representative titration curves showing changes in solution pH in response to addition of a strong acid for IAJD molecules.

FIG. 69 depicts representative titration curves showing changes in solution pH in response to addition of a strong acid for IAJD molecules.

FIG. 70 depicts representative titration curves showing changes in solution pH in response to addition of a strong acid for IAJD molecules.

FIG. 71 depicts representative titration curves showing changes in solution pH in response to addition of a strong acid for IAJD molecules.

FIG. 72 depicts representative titration curves showing changes in solution pH in response to addition of a strong acid for IAJD molecules.

FIG. 73 depicts representative results relating structure and activity of IAJDs.

FIG. 74 depicts schematic representations of IAJDs with nonsymmetric alkyl chains and representative results demonstrating selective delivery of Luc-mRNA to spleens and lymph nodes in vivo by DNPs and less-selective delivery of Luc-mRNA to organs in vivo by DNPs.

FIG. 75 depicts representative results demonstrating the ratios between in vivo activities of nonsymmetric IAJDs with one C18 alkyl chain and corresponding symmetric IAJDs with two identical C18 alkyl chains.

FIG. 76 depicts representative results demonstrating dimensions of DNPs assembled from IAJD125 in 1% fetal bovine serum.

FIG. 77 depicts representative results demonstrating dimensions of DNPs assembled from IAJD155 in 1% fetal bovine serum.

FIG. 78 depicts representative DLS data of DNP 125 and 178 before and after dialysis in 1×PBS buffer for 3 h.

FIG. 79 depicts a schematic representation of glycerol-amphiphilic Ds.

FIG. 80 depicts schematic representations of R-, S-, rac- and achiral glycerol with two protected OH and homochiral (1R, 1S, 2R, 2S), racemic (1rac, 2rac), and achiral (3, 4) glycerol-JDs.

FIG. 81 depicts a schematic representation of synthesis of Glycerol-JDs from Library 1. Reagents and conditions: (i) DCC, DPTS, DCM, 23° C., 12 h; (ii) H₂, Pd/C, EtOAc, 23° C., 8 h; (iii) 1 M HCl, MeOH, 23° C., 1 h.

FIG. 82 depicts a schematic representation of synthesis of Glycerol-JDs from Library 2. Reagents and conditions: (i) DCC, DPTS, DCM, 23° C., 12 h; (ii) H₂, Pd/C, EtOAc, 23° C., 8 h; (iii) 1 M HCl, 1,4-dioxane, 60° C., 8 h.

FIG. 83 depicts a schematic representation of synthesis of Glycerol-Achiral Ds. Reagents and conditions: (i) DCC, DPTS, DCM, 23° C., 12 h; (ii) H₂, Pd/C, EtOAc, 23° C., 8 h; (iii) 1 M HCl, 1,4-dioxane, 60° C., 8 h.

FIG. 84 depicts representative results demonstrating the concentration dependence of diameter (Dₕ, in nm) and square diameter (Dₕ²) of DSs assembled by glycerol-JDs in water. Top two left graphs correspond to glycerol-JDs of library 1. Bottom two right graphs correspond to achiral glycerol-JDs 3 and 4. The remaining graphs correspond to glycerol-JDs of library 2.

FIG. 85 depicts representative cryo-TEM images of DSs assembled by glycerol-JD 2R, 2S, and 2rac.

FIG. 86 depicts representative cryo-TEM images of DSs self-assembled by glycerol-based JD 1R (a), 1S (b) and 1rac (c). Scale bar is 100 nm.

FIG. 87 depicts representative cryo-TEM images of DSs self-assembled by glycerol-based achiral JD 3.

FIG. 88 depicts representative cryo-TEM images of DSs self-assembled by glycerol-based achiral JD 4.

FIG. 89 depicts representative results demonstrating histograms of normalized frequency of number of vesicle layers of DSs assembled from glycerol-JD 1R, 1S, 1rac, 2R, 2S, and 2rac by statistical analysis.

FIG. 90 depicts representative ¹H NMR spectrum of (3,5)-12G1-GC-(R)-BMPA-(3,4,5)-3EO-G1 (1R). CDCl₃, 500 MHz, 298 K. Asterisked signals at δ 7.26 ppm and 2.04 ppm are due to partially nondeuterated residues of CDCl₃ and EtOAc respectively.

FIG. 91 depicts representative ¹³C NMR spectrum of (3,5)-12G1-GC-(R)-BMPA-(3,4,5)-3EO-G1 (1R). CDCl₃, 500 MHz, 298 K. The asterisked signal at δ 77.16 ppm is due to CDCl₃.

FIG. 92 depicts representative ¹H NMR spectrum of (3,5)-12G1-GC-(S)-BMPA-(3,4,5)-3EO-G1 (1S). CDCl₃, 500 MHz, 298 K. Asterisked signals at δ 7.26 ppm and 2.04 ppm are due to partially nondeuterated residues of CDCl₃ and EA respectively.

FIG. 93 depicts representative ¹³C NMR spectrum of (3,5)-12G1-GC-(S)-BMPA-(3,4,5)-3EO-G1 (1S). CDCl₃, 500 MHz, 298 K. The asterisked signal at δ 77.16 ppm is due to CDCl₃.

FIG. 94 depicts representative ¹H NMR spectrum of (3,5)-12G1-GC-(rac)-BMPA-(3,4,5)-3EO-G1 (1rac). CDCl₃, 500 MHz, 298 K. Asterisked signals at δ 7.26 ppm, 5.30 ppm and 1.66 ppm are due to partially nondeuterated residues of CDCl₃, DCM, and water respectively.

FIG. 95 depicts representative ¹³C NMR spectrum of (3,5)-12G1-GC-(rac)-BMPA-(3,4,5)-3EO-G1 (1rac). CDCl₃, 500 MHz, 298 K. The asterisked signal at δ 77.16 ppm is due to CDCl₃.

FIG. 96 depicts representative ¹H NMR spectrum of (3,5)-12G1-BMPA-GC-(R)-(3,4,5)-3EO-G1 (2R). CDCl₃, 500 MHz, 298 K. Asterisked signals at δ 7.26 ppm and 1.68 ppm are due to partially nondeuterated residues of CDCl₃ and water respectively.

FIG. 97 depicts representative ¹³C NMR spectrum of (3,5)-12G1-BMPA-GC-(R)-(3,4,5)-3EO-G1 (2R). CDCl₃, 500 MHz, 298 K. The asterisked signal at δ 77.16 ppm is due to CDCl₃.

FIG. 98 depicts representative ¹H NMR spectrum of (3,5)-12G1-BMPA-GC-(S)-(3,4,5)-3EO-G1 (2S). CDCl₃, 500 MHz, 298 K. Asterisked signals at δ 7.26 ppm and 1.65 ppm are due to partially nondeuterated residues of CDCl₃ and water respectively.

FIG. 99 depicts representative ¹³C NMR spectrum of (3,5)-12G1-BMPA-GC-(S)-(3,4,5)-3EO-G1 (2S). CDCl₃, 500 MHz, 298 K. The asterisked signal at δ 77.16 ppm is due to CDCl₃.

FIG. 100 depicts representative ¹H NMR spectrum of (3,5)-12G1-BMPA-GC-(rac)-(3,4,5)-3EO-G1 (2rac). CDCl₃, 500 MHz, 298 K. Asterisked signals at δ 7.26 ppm and 1.60 ppm are due to partially nondeuterated residues of CDCl₃ and water respectively.

FIG. 101 depicts representative ¹³C NMR spectrum of (3,5)-12G1-BMPA-GC-(rac)-(3,4,5)-3EO-G1 (2rac). CDCl₃, 500 MHz, 298 K. The asterisked signal at δ 77.16 ppm is due to CDCl₃.

FIG. 102 depicts representative ¹H NMR spectrum of (3,5)-12G1-GC-(achiral)-BMPA-(3,4,5)-3EO-G2 (3). CDCl₃, 500 MHz, 298 K. Asterisked signals at δ 7.26 ppm, 2.04 ppm and 1.66 ppm are due to partially nondeuterated residues of CDCl₃, EtOAc, and water respectively.

FIG. 103 depicts representative ¹³C NMR spectrum of (3,5)-12G1-GC-(achiral)-BMPA-(3,4,5)-3EO-G2 (3). CDCl₃, 500 MHz, 298 K. The asterisked signal at δ 77.16 ppm is due to CDCl₃.

FIG. 104 depicts representative ¹H NMR spectrum of (3,5)-12G2-BMPA-GC-(achiral)-(3,4,5)-3EO-G1 (4). CDCl₃, 500 MHz, 298 K. Asterisked signals at δ 7.26 ppm, 5.30 ppm and 1.67 ppm are due to partially nondeuterated residues of CDCl₃, DCM, and water respectively.

FIG. 105 depicts representative ¹³C NMR spectrum of (3,5)-12G2-BMPA-GC-(achiral)-(3,4,5)-3EO-G1 (4). CDCl₃, 500 MHz, 298 K. The asterisked signal at δ 77.16 ppm is due to CDCl₃.

FIG. 106 depicts representative MALDI-TOF-MS spectra of glycerol-based JDs from library 1.

FIG. 107 depicts representative MALDI-TOF-MS spectra of glycerol-based JDs from library 2.

FIG. 108 depicts representative MALDI-TOF-MS spectra of glycerol-based achiral JDs 3 (left) and 4 (right).

FIG. 109 depicts representative HPLC traces of glycerol-based JDs from library 1.

FIG. 110 depicts representative HPLC traces of glycerol-based JDs from library 2.

FIG. 111 depicts representative HPLC traces of glycerol-based achiral JDs 3 (left) and 4 (right).

FIG. 112 depicts representative results demonstrating normal Q-Q plot of sample 1rac. The distribution of 1rac is normal-like.

FIG. 113 depicts representative results demonstrating normal Q-Q plot of sample 2rac. The distribution of 2rac is normal-like.

### DETAILED DESCRIPTION

The present invention is based, in part, on the unexpected results that nanoparticles comprising at least one ionizable amphiphilic Janus dendrimer having the structure of Formula (I) effectively and efficiently delivered an agent to a target of interest. Thus, in one aspect, the present invention relates to an ionizable amphiphilic Janus dendrimer having the structure of Formula (I).

In another aspect, the present invention relates to a nanoparticle comprising at least one ionizable amphiphilic Janus dendrimer of the present invention. In some embodiments, the nanoparticle further comprises at least one agent. In some embodiment, the nanoparticle further comprises at least one agent that is encapsulated by the ionizable amphiphilic Janus dendrimer of the present invention. In another aspect, the present invention relates to a composition comprising at least one ionizable amphiphilic Janus dendrimer of the present invention or a nanoparticle thereof. In some embodiments, the composition is a vaccine.

In one aspect, the present invention relates to methods of delivering an agent to a target of interest using at least one ionizable amphiphilic Janus dendrimer of the present invention or a nanoparticle or a composition thereof. In another aspect, the present invention relates to methods of preventing or treating a disease or disorder in a subject using at least one ionizable amphiphilic Janus dendrimer of the present invention or a nanoparticle or a composition thereof. In another aspect, the present invention relates to methods of inducing an adaptive immune response in a subject using at least one ionizable amphiphilic Janus dendrimer of the present invention or a nanoparticle or a composition thereof.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element. “About” as used herein when referring to a measurable value, for example numerical values and/or ranges, such as an amount, a temporal duration, and the like, is meant to encompass variations of 20%, ±10%, ±5%, ±1%, or +0.1% from the specified value, as such variations are appropriate to perform the disclosed methods. For example, “about 40 [units]” may mean within 25% of 40 (e.g., from 30 to 50), within +20%, 15%, 10%, 9%, 8%, 7%, 6%, 5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

The term “compound,” as used herein, unless otherwise indicated, refers to any specific chemical compound disclosed herein. In one embodiment, the term also refers to stereoisomers and/or optical isomers (including racemic mixtures) or enantiomerically enriched mixtures of disclosed compounds.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, prodrug, or other variant of the reference molecule.

As used herein, the term “prodrug” refers to an agent that is converted into the parent drug in vivo. For example, the term “prodrug” refers to a derivative of a known direct acting drug, which derivative has enhanced delivery characteristics and therapeutic value as compared to the drug, and is transformed into the active drug by an enzymatic or chemical process. In some embodiments, “prodrug” refers to an inactive or relatively less active form of an active agent that becomes active by undergoing a chemical conversion through one or more metabolic processes. In one embodiment, upon in vivo administration, a prodrug is chemically converted to the biologically, pharmaceutically, or therapeutically active form of the compound. In another embodiment, a prodrug is enzymatically metabolized by one or more steps or processes to the biologically, pharmaceutically, or therapeutically active form of the compound. For example, the present compounds can be administered to a subject as a prodrug that includes an initiator bound to an active agent, and, by virtue of being degraded by a metabolic process, the active agent is released in its active form.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

As used herein, the term “alkyl,” by itself or as part of another substituent means, unless otherwise stated, a straight or branched chain hydrocarbon having the number of carbon atoms designated (i.e., C₁₋₅₀ means one to fifty carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(═O)OH, trifluoromethyl, —C—N, —C(═O)O(C₁-C₄)alkyl, —C(═O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

The term “amino” refers to a group of the formula —NRaRa, —NHRa, or —NH₂, where each Ra is, independently, an alkyl, alkenyl or alkynyl group as defined above containing 1 to 20 carbon atoms. Unless stated otherwise specifically in the specification, an alkylamino group can be optionally substituted.

The term “hydroxy” or “hydroxyl” refers to a group of the formula OH group.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo”, “halide”, or “halogen” alone or as part of another substituent means, unless otherwise stated, a fluorine, chlorine, bromine, or iodine atom.

“Haloalkyl” or “alkylhalide” refers to an alkyl radical, as defined above, that is substituted by one or more halo radicals, as defined above, e.g., trifluoromethyl, difluoromethyl, trichloromethyl, 2,2,2 trifluoroethyl, 1,2 difluoroethyl, 3 bromo 2 fluoropropyl, 1,2 dibromoethyl, and the like. Unless stated otherwise specifically in the specification, a haloalkyl group can be optionally substituted.

As used herein, the term “cycloalkyl” refers to a mono cyclic or polycyclic non-aromatic 25 radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

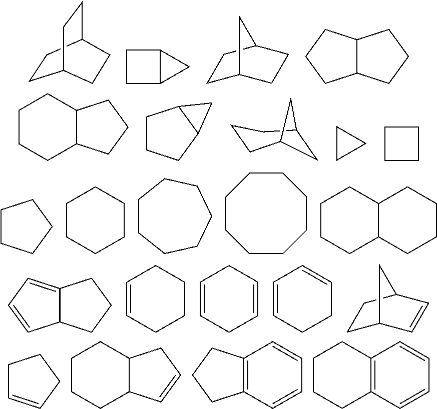

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heterocycloalkyl” or “heterocyclyl” refers to a cyclic group containing one to four ring heteroatoms each selected from O, S, and N. In one embodiment, each heterocycloalkyl group has from 4 to 10 atoms in its ring system, with the proviso that the ring of said group does not contain two adjacent O atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are.

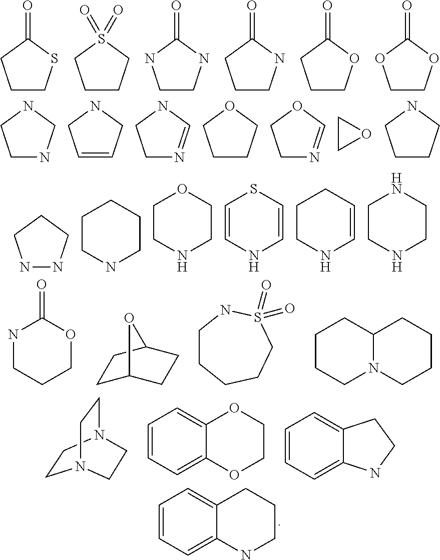

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized 11 (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

“Aralkyl” or “arylalkyl” refers to a radical of the formula —R_{b}—R_{c} where R_{b} is an alkylene group as defined above and Re is one or more aryl radicals as defined above, for example, benzyl, diphenylmethyl and the like. Unless stated otherwise specifically in the specification, an aralkyl group can be optionally substituted. For example, as used herein, the term “aryl-(C₁-C₃)alkyl” means a functional group wherein a one- to three-carbon alkylene chain is attached to an aryl group, e.g., —CH₂CH₂-phenyl, —CH₂-phenyl (benzyl), aryl-CH₂— and aryl-CH(CH₃)—. The term “substituted aryl-(C₁-C₃)alkyl” means an aryl-(C₁-C₃)alkyl functional group in which the aryl group is substituted. Similarly, the term “heteroaryl-(C₁-C₃)alkyl” means a functional group wherein a one to three carbon alkylene chain is attached to a heteroaryl group, e.g., —CH₂CH₂-pyridyl. The term “substituted heteroaryl-(C₁-C₃)alkyl” means a heteroaryl-(C₁-C₃)alkyl functional group in which the heteroaryl group is substituted.

As used herein, the term “heteroaryl” or “heteroaromatic” refers to a heterocycle having aromatic character. A polycyclic heteroaryl may include one or more rings that are partially saturated. Examples include the following moieties:

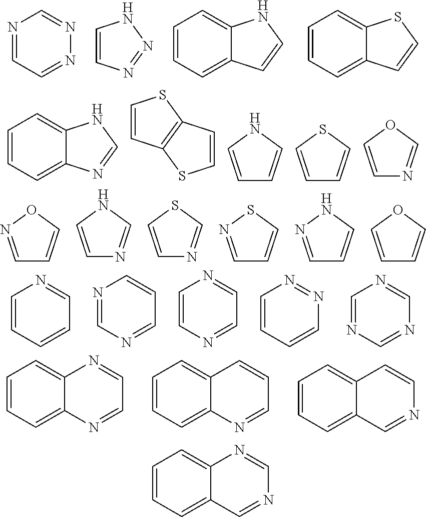

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, S(═O)₂N[H, alkyl, or aryl], —C(═O)NH[substituted or unsubstituted alkyl, or substituted or unsubstituted phenyl], —C(═O)N[H or substituted or unsubstituted alkyl or aryl]2, —OC(═O)N[substituted or unsubstituted alkyl]2, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]2, and —C(NH₂)[substituted or unsubstituted alkyl]2. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. As used herein, where a substituent is an alkyl or alkoxy group, the carbon chain may be branched, straight or cyclic.

Several references to integers and R, R¹, R², R³, R⁴, R⁵, R⁶, etc. are made in chemical structures and moieties disclosed and described herein. Any description of integers and R, R¹, R², R³, R⁴, R⁵, R⁶, etc. in the specification is applicable to any structure or moiety reciting integers and R, R¹, R², R³, R⁴, R⁵, R⁶, etc. respectively.

The term “nanoparticle” refers to a particle having at least one dimension on the order of nanometers (e.g., 1-1,000 nm), which includes one or more amphiphilic Janus dendrimer of Formula (I). In some embodiments, nanoparticles are included in a formulation comprising a nucleoside-modified RNA as described herein. In some embodiments, such nanoparticles an ionizable hydrophilic group and a lipophilic (hydrophobic) group. In one embodiment, the nanoparticles further comprise one or more excipient selected from neutral lipids, charged lipids, steroids and polymer conjugated lipids. In one embodiment, the nanoparticles do not comprise additional excipients. In one embodiment, the nanoparticles do not comprise any of additional lipids, additional cationic polymers, steroids, neutral lipids, charged lipids, or polymer conjugated lipids, besides the at least one compound of Formula (I). In some embodiments, the nucleoside-modified RNA is encapsulated in the lipid portion of the nanoparticle or an aqueous space enveloped by some or all of the lipid portion of the nanoparticle, thereby protecting it from enzymatic degradation or other undesirable effects induced by the mechanisms of the host organism or cells, e.g., an adverse immune response.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “adjuvant” as used herein is defined as any molecule to enhance an antigen-specific adaptive immune response.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Homologous” as used herein, refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Immunogen” refers to any substance introduced into the body in order to generate an immune response. That substance can a physical molecule, such as a protein, or can be encoded by a vector, such as DNA, mRNA, or a virus.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translation by translational machinery in a cell. For example, an mRNA where all of the uridines have been replaced with pseudouridine, 1-methyl pseudouridine, or another modified nucleoside.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27:196-197).

In certain embodiments, “pseudouridine” refers, in another embodiment, to m¹acp³ψ (1-methyl-3-(3-amino-3-carboxypropyl) pseudouridine. In another embodiment, the term refers to m¹ψ (1-methylpseudouridine). In another embodiment, the term refers to ψm (2′-O-methylpseudouridine). In another embodiment, the term refers to m⁵D (5-methyldihydrouridine). In another embodiment, the term refers to m³ψ (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the invention.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “amino acid”, “amino acidic monomer”, or “amino acid residue” refer to any of the twenty naturally occurring amino acids including synthetic amino acids with unnatural side chains and including both D and L optical isomers.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

The term “therapeutic” as used herein means a treatment and/or prophylaxis. A therapeutic effect is obtained by suppression, diminution, remission, or eradication of at least one sign or symptom of a disease or disorder state.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the nanocluster. The active agent-nanocluster combination may be coated further to encapsulate the agent-nanocluster combination and may be directed to a target by functionalizing the nanocluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

An “effective amount” as used herein, means an amount which provides a therapeutic or prophylactic benefit.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

To “treat” a disease as the term is used herein, means to reduce the frequency or severity of at least one sign or symptom of a disease or disorder experienced by a subject.

As used herein, “treating a disease or disorder” means reducing the frequency with which a symptom of the disease or disorder is experienced by a patient. Disease and disorder are used interchangeably herein.

A disease or disorder is “alleviated” if the severity of a sign or symptom of the disease or disorder, the frequency with which such a sign or symptom is experienced by a patient, or both, is reduced.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Parenteral” administration of a composition includes, e.g., subcutaneous (s.c.), intravenous (i.v.), intramuscular (i.m.), or intrasternal injection, or infusion techniques.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the unexpected results that nanoparticles comprising at least one ionizable amphiphilic Janus dendrimer having the structure of Formula (I) effectively and efficiently delivered an agent to a target of interest. Thus, in one aspect, the present invention relates to an ionizable amphiphilic Janus dendrimer having the structure of Formula (I). In another aspect, the present invention relates to a nanoparticle comprising at least one ionizable amphiphilic Janus dendrimer of the present invention. In some embodiments, the nanoparticle further comprises at least one agent. In some embodiment, the nanoparticle further comprises at least one agent that is encapsulated by the ionizable amphiphilic Janus dendrimer of the present invention. In another aspect, the present invention relates to a composition comprising at least one ionizable amphiphilic Janus dendrimer of the present invention or a nanoparticle thereof. In some embodiments, the composition is a vaccine.

In one aspect, the present invention relates to methods of delivering an agent to a target of interest using at least one ionizable amphiphilic Janus dendrimer of the present invention or a nanoparticle or a composition thereof. In another aspect, the present invention relates to methods of preventing or treating a disease or disorder in a subject using at least one ionizable amphiphilic Janus dendrimer of the present invention or a nanoparticle or a composition thereof. In another aspect, the present invention relates to methods of inducing an adaptive immune response in a subject using at least one ionizable amphiphilic Janus dendrimer of the present invention or a nanoparticle or a composition thereof.

### Compounds of the Invention

In one aspect, the invention relates to amphiphilic Janus dendrimers of Formula (I):

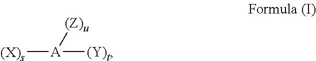

or a racemate, an enantiomer, a diastereomer, a pharmaceutically acceptable salt, or a derivative thereof. In various embodiments, the amphiphilic Janus dendrimer is an ionizable amphiphilic Janus dendrimer.

In some embodiments, A is a polyvalent group comprising at least one selected from the following structures:

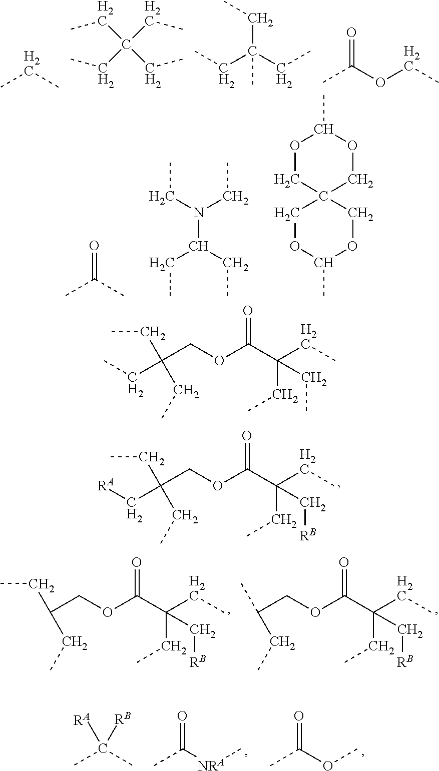

or any combination thereof. In some embodiments, A is a polyvalent group comprising at least one selected from

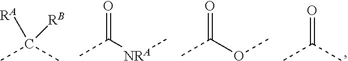

or any combination thereof. In some embodiments, A is optionally substituted.

In some embodiments, dashed lines represent a binding site of one of X, Y, or Z.

In some embodiments, X is a hydrophilic group comprising at least one amine. In some embodiments, the hydrophilic group is optionally substituted. In some embodiments, the amine is optionally substituted.

In some embodiments, Y is a lipophilic group comprising at least one C₁-C₅₀ alkyl chain. In some embodiments, Y is a lipophilic group comprising at least one C₁-C₃₀ alkyl chain. In some embodiments, the lipophilic group is optionally substituted. In some embodiments, the C₁-C₅₀ alkyl chain is optionally substituted.

In some embodiments, Z is a group comprising at least one selected from ethylene glycol, diethylene glycol, triethylene glycol, or polyethylene glycol chain. In some embodiments, Z is optionally substituted. In some embodiments, the ethylene glycol, diethylene glycol, triethylene glycol, and/or polyethylene glycol chain are optionally substituted.

In some embodiments, R^{A} is selected from hydrogen, halide, hydroxy, C₁-C₅₀-alkyl, C₁-C₅₀-alkyl halide, C₁-C₅₀-alkoxy, C₁-C₅₀-alkoxy halide, or any combination thereof. In some embodiments, R^{A} is selected from hydrogen, halide, hydroxy, C₁-C₃₀-alkyl, C₁-C₃₀-alkyl halide, C₁-C₃₀-alkoxy, C₁-C₃₀-alkoxy halide, or any combination thereof. In some embodiments, the hydroxy, C₁-C₅₀-alkyl, C₁-C₅₀-alkyl halide, C₁-C₅₀-alkoxy, and/or C₁-C₅₀-alkoxy halide are optionally substituted.

In some embodiments, R^{B} is selected from hydrogen, halide, hydroxy, C₁-C₅₀-alkyl, C₁-C₅₀-alkyl halide, C₁-C₅₀-alkoxy, C₁-C₅₀-alkoxy halide, or any combination thereof. In some embodiments, R^{B} is selected from hydrogen, halide, hydroxy, C₁-C₃₀-alkyl, C₁-C₃₀-alkyl halide, C₁-C₃₀-alkoxy, C₁-C₃₀-alkoxy halide, or any combination thereof. In some embodiments, the hydroxy, C₁-C₅₀-alkyl, C₁-C₅₀-alkyl halide, C₁-C₅₀-alkoxy, and/or C₁-C₅₀-alkoxy halide are optionally substituted.

In some embodiments, s is an integer from 0 to 5. In some embodiments, s is an integer from 1 to 5. For example, in one embodiment, s is an integer 5. In one embodiment, s is an integer 4. In one embodiment, s is an integer 3. In one embodiment, s is an integer 2. In one embodiment, s is an integer 1. In one embodiment, s is an integer 0.

In some embodiments, t is an integer from 0 to 5. In some embodiments, t is an integer from 1 to 5. For example, in one embodiment, t is an integer 5. In one embodiment, t is an integer 4. In one embodiment, t is an integer 3. In one embodiment, t is an integer 2. In one embodiment, t is an integer 1. In one embodiment, t is an integer 0.

In some embodiments, u is an integer from 0 to 4. In some embodiments, u is an integer from 1 to 4. For example, in one embodiment, u is an integer 4. In one embodiment, u is an integer 3. In one embodiment, u is an integer 2. In one embodiment, u is an integer 1. In one embodiment, u is an integer 0.

In some embodiments, the sum of s, t, and u is equal to the valency of A. For example, in one embodiment, the valency of A is 5, and the sum of s, t, and u is 5. In one embodiment, the valency of A is 4, and the sum of s, t, and u is 4. In one embodiment, s and t are each 2. In one embodiment, s is 1 and t is 3. In one embodiment, s is 3 and t is 1. In one embodiment, s is 1, t is 1, and u is 2. In one embodiment, s is 2, t is 1, and u is 1. In one embodiment, s is 1, t is 2, and u is 1. In one embodiment, the valency of A is 2, and the sum of s, t, and u is 2. In one embodiment, s and t are each 1.

For example, in some embodiments, A is represented by

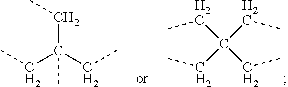

and s and t are each 2.

In some embodiments, A is represented by

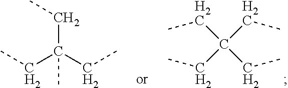

t is 2; s is 1; and u is 0 or 1.

In some embodiments, A is represented by

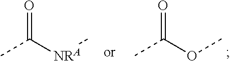

and s and t are each 1.

In one embodiment, u is 0. Thus, in one embodiment, wherein the amphiphilic Janus dendrimer having the structure of Formula (I) is an amphiphilic Janus dendrimer having the structure of Formula (II):

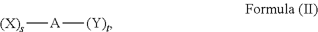

or a racemate, an enantiomer, a diastereomer, a pharmaceutically acceptable salt, or a derivative thereof.

In one embodiment, X is a hydrophilic group comprising one amine. In one embodiment, X is a hydrophilic group comprising two amines. In one embodiment, X is a hydrophilic group comprising three amines. In one embodiment, X comprises at least one primary amine. In one embodiment, X comprises at least one secondary amine. In one embodiment, X comprises at least one tertiary amine. For example, in one embodiment, X comprises at least two tertiary amines.

In one embodiment, X comprises at least one amine which is protonated under biological conditions. In one embodiment, X comprises at least one amine which has a formal charge of +1 under biological conditions. In one embodiment, X comprises at least one carbohydrate.

In one embodiment, each occurrence of X is independently selected from the following structures:

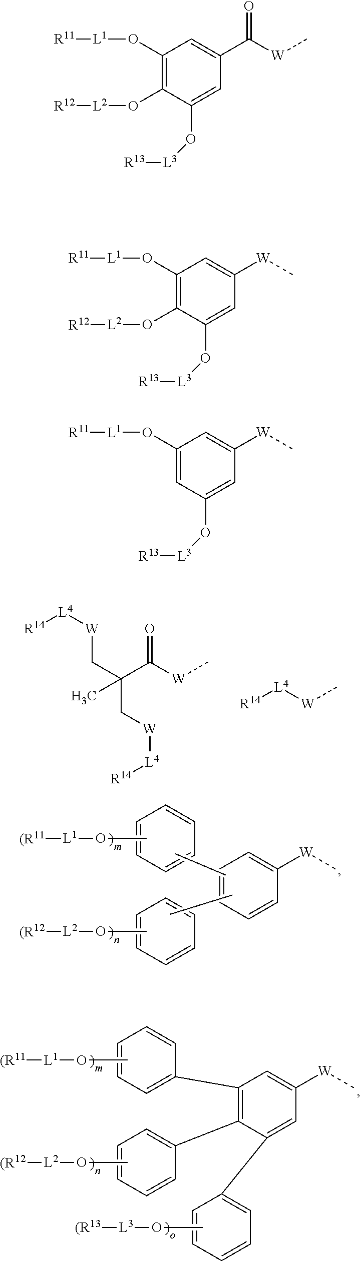

or any combination thereof.

In some embodiments, dashed lines indicate the connection to A.

In some embodiments, each occurrence of m, n, o is independently an integer from 1 to 5.

In some embodiments, each occurrence of W is independently selected from C═O, C(R^{W})(R^{W}), NR^{W}, O, or S. In some embodiments, each occurrence of R^{W} is independently selected from hydrogen, halide, hydroxy, alkyl, alkyl halide, aryl, aryl halide, alkoxy, alkoxy halide, or any combination thereof. In one embodiment, each occurrence of W is NH. In one embodiment, each occurrence of W is O. In one embodiment, at least one occurrence of W is NH and at least occurrence of W is O.

In some embodiments, each occurrence of L¹, L², L³, and L⁴ is independently a covalent bond or a divalent linking group selected from alkylene, cycloalkylene, heteroalkylene, heterocycloalkylene, alkenylene, alkynylene, arylene, heteroarylene, silyl, amine, amide, ester, ether, carbonyl, carbamate, thioether, thioester, disulfide, hydrazine, urea, thiourea, phosphate, poly(alkyl ether), heteroatom, or any combination thereof. In one embodiment, at least one occurrence of L¹, L², L³, and L⁴ is a poly(alkyl ether) or an oligo(alkyl ether). In one embodiment, each occurrence of L¹, L², L³, and L⁴ is polyethylene glycol (PEG) or oligoethyleneoxide. In one embodiment, each occurrence of L¹, L², L³, and L⁴ independently has the structure—[CH₂CH₂O]ₙ—; wherein n is an integer between 0 and 10.

In some embodiments, each occurrence of R¹¹, R¹², R¹³, and R¹⁴ is independently selected from hydrogen, deuterium, alkyl, aryl, heteroaryl, cycloalkyl, alkoxy, phenoxy, amine, heterocycloalkyl, carbonyl, or any combination thereof. In some embodiments, at least one of R¹¹, R¹², R¹³, or R¹⁴ comprises an amine. In one embodiment, one of R¹¹, R¹², and R¹³ comprises an amine. In one embodiment, two of R¹¹, R¹², and R¹³ comprises an amine. In one embodiment, three of R¹¹, R¹², and R¹³ comprises an amine. In one embodiment, at least one of R¹¹, R¹², and R¹³ comprises two amines. In one embodiment the amine of one of R¹¹, R¹², and R¹³ is protonated under biological conditions. In one embodiment, any of R¹¹, R¹², and R¹³ that does not comprise an amine comprises an alkyl group, an aryl group, or a combination thereof.

In one embodiment, at least one occurrence of R¹¹, R¹², R¹³, or R¹⁴ has the structure

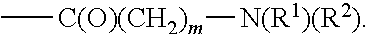

In some embodiments, each occurrence of m is an integer from 1 to 10. In one embodiment, m is 1, 2, 3, 4, 5, 6, 7, 8, 9, or 10.

In some embodiments, each occurrence of R¹ and R² is independently selected from the hydrogen, deuterium, alkyl, aryl, cycloalkyl, amine, heterocycloalkyl, carbonyl, or any combination thereof. In some embodiments, R¹ and R² may together form a ring.

In one embodiment, R¹ and R² are each alkyl. In one embodiment, R¹ and R² are each methyl. In one embodiment, R¹ and R² together form a 6-membered heterocyclic ring. In one embodiment, R¹ and R² together form a piperidine ring with the N to which they are bound. In one embodiment, R¹ and R² together form an N-alkyl piperazine ring with the N to which they are bound. In one embodiment, R¹ and R² are each hydrogen.

In one embodiment, at least one occurrence of R¹¹, R¹², R¹³, or R¹⁴ has the structure —C(O)(CR⁵R⁶)ₘ—N(R¹)(R²).

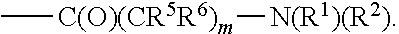

In some embodiments, each occurrence of m is an integer from 1 to 10. In one embodiment, m is 1, 2, 3, 4, 5, 6, 7, 8, 9, or 10.

In some embodiments, each occurrence of R¹ and R² is independently selected from hydrogen, deuterium, alkyl, aryl, cycloalkyl, amine, heterocycloalkyl, carbonyl, or any combination thereof.

In some embodiments, each occurrence of R⁵ and R⁶ is independently selected from hydrogen, deuterium, alkyl, aryl, heteroaryl, cycloalkyl, alkoxy, phenoxy, amine, heterocycloalkyl, carbonyl, or any combination thereof. In some embodiments, any two or more of R¹, R², R⁵, and R⁶ may together form a ring.

In one embodiment, the group having the structure —C(O)(CR⁵R⁶)ₘ—N(R¹)(R²) is derived from an amino acid. In one embodiment, the amino acid is a canonical amino acid. In one embodiment, the amino acid is a non-canonical amino acid. In one embodiment, the amino acid is a beta-amino acid (i.e., m is at least 2, and one geminal pair of R⁵ and R⁶ is H). In one embodiment, one of R⁵ and R⁶ is H, and the other of R⁵ and R⁶ is not H. In one embodiment, the group may be chiral. In one embodiment, the group may be enantiomerically enriched or enantiomerically pure. In one embodiment, the group, the group may be racemic.

In one embodiment, each occurrence of R¹¹, R¹², R¹³, and R¹⁴ is independently selected from the following structures:

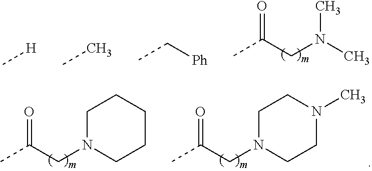

In some embodiments, each occurrence of m is an integer from 1 to 10. In one embodiment, m is 1, 2, 3, 4, 5, 6, 7, 8, 9, or 10.

In one embodiment, each occurrence of X is independently selected from the following structures:

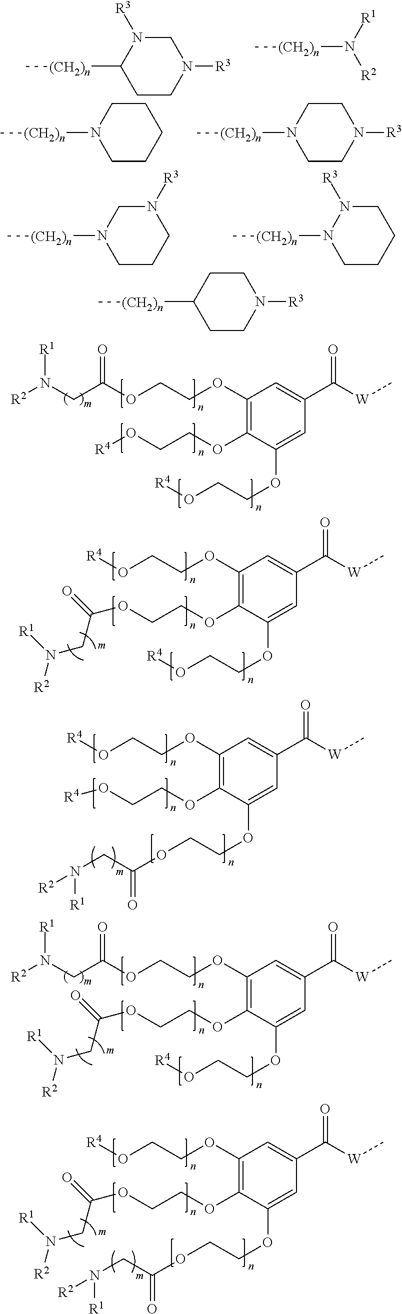

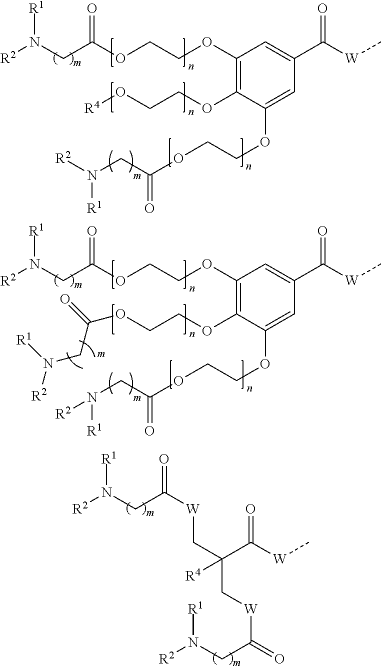

or any combination thereof.

In some embodiments, each occurrence of n is independently an integer from 1 to 20. In 5 some embodiments, each occurrence of n is independently an integer from 1 to 10.

In some embodiments, each occurrence of m is independently an integer from 1 to 20. In some embodiments, each occurrence of m is independently an integer from 1 to 10.

In some embodiments, each occurrence of R¹ and R² is independently selected from hydrogen, deuterium, alkyl, aryl, cycloalkyl, amine, heterocycloalkyl, carbonyl, or any combination thereof. In some embodiments, R¹ and R² may together form a ring.

In some embodiments, each occurrence of R³ is independently selected from hydrogen, (CH₂)ₙ, (CH₂)ₙ—OH, or any combination thereof.

In some embodiments, each occurrence of R⁴ is independently selected from hydrogen, deuterium, alkyl, aryl, or any combinations thereof.

For example, in some embodiments, each occurrence of X is independently selected from

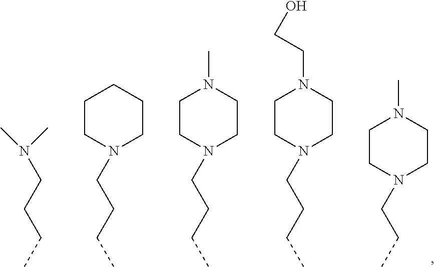

or any combination thereof.

In one embodiment, Y is a lipophilic (hydrophobic) group comprising at least one C₁-C₅₀ alkyl chain. In one embodiment, Y is a lipophilic (hydrophobic) group comprising at least one C₁-C₃₀ alkyl chain. In one embodiment, Y is a lipophilic (hydrophobic) group comprising at least one C₄-C₃₀ alkyl chain. For example, in one embodiment, Y is a lipophilic (hydrophobic) group comprising at least two C₁-C₃₀ alkyl chain. In one embodiment, Y is a lipophilic (hydrophobic) group comprising at least two C₁-C₃₀ alkyl chain having differing numbers of carbon atoms.

In one embodiment, Y is a lipophilic group further comprising at least one linking group selected from the group consisting of alkylene, cycloalkylene, heteroalkylene, heterocycloalkylene, alkenylene, alkynylene, arylene, heteroarylene, silyl, amine, amide, ester, ether, carbonyl, carbamate, thioether, thioester, disulfide, hydrazine, urea, thiourea, phosphate, poly(alkyl ether), heteroatom, or any combination thereof. In one embodiment, Y comprises alkylene, arylene, alkenylene, alkynylene, disulfide, ether, or any combination thereof. In one embodiment, Y does not comprise an amine. In one embodiment, Y is a lipophilic group comprising at least one C₆ alkyl chain, at least one C₇ alkyl chain, at least one C₈ alkyl chain, at least one C₉ alkyl chain, at least one C₁₀ alkyl chain, at least one C₁₁ alkyl chain, at least one C₁₂ alkyl chain, at least one C₁₃ alkyl chain, at least one C₁₄ alkyl chain, at least one C₁₅ alkyl chain, at least one C₂₀ alkyl chain, at least one C₂₁ alkyl chain, at least one C₂₃ alkyl chain, at least one C₂₇ alkyl chain, at least one C₃₀ alkyl chain, at least one C₃₅ alkyl chain, at least one C₄₀ alkyl chain, or at least one C₅₀ alkyl chain. In one embodiment, Y comprises at least one carbohydrate.

In one embodiment, each occurrence of Y is independently selected from the following structures:

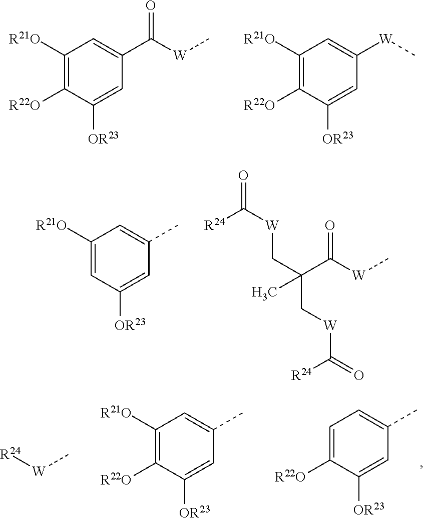

or any combination thereof.

In some embodiments, dashed lines indicate the connection to A.

In some embodiments, each occurrence of W is independently selected from C═O, C(R^{W})(R^{W}), NR^{W}, O, or S. In some embodiments, each occurrence of R^{W} is independently selected from hydrogen, halide, hydroxy, alkyl, alkyl halide, aryl, aryl halide, alkoxy, alkoxy halide, or any combination thereof. In one embodiment, each occurrence of W represents O. In one embodiment, each occurrence of W represents NH. In one embodiment, at least one occurrence of W represents O and at least one occurrence of W represents NH.

In some embodiments, each occurrence of R²¹, R²², R²³, and R²⁴ independently comprises C₁-C₅₀-alkyl. In some embodiments, each occurrence of R²¹, R²², R²³, and R²⁴ independently comprises a linear or branched C₁-C₅₀-alkyl. In some embodiments, each occurrence of R²¹, R²², R²³, and R²⁴ independently comprises C₁-C₃₀-alkyl. In some embodiments, each occurrence of R²¹, R²², R²³, and R²⁴ independently comprises a linear or branched C₁-C₃₀ alkyl. In one embodiment, each occurrence of R²¹, R²², R²³, and R²⁴ independently represents C₈-C₁₂ alkyl.

In one embodiment, each occurrence of Y is independently selected from the following structures:

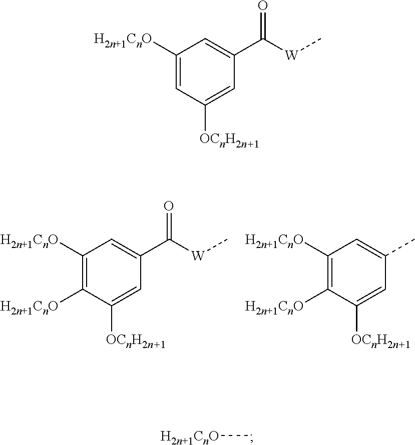

In some embodiments, each occurrence of W is independently selected from C═O, C(R^{W})(R^{W}), NR^{W}, O, or S. In some embodiments, each occurrence of R^{W} is independently selected from hydrogen, halide, hydroxy, alkyl, alkyl halide, aryl, aryl halide, alkoxy, alkoxy halide, or any combination thereof. In one embodiment, each occurrence of W represents O. In one embodiment, each occurrence of W represents NH. In one embodiment, at least one occurrence of W represents O and at least one occurrence of W represents NH.

In some embodiments, n is an integer from 1 to 30. In some embodiments, n is an integer from 6 to 18.

In some embodiments, each occurrence of Y is independently selected from

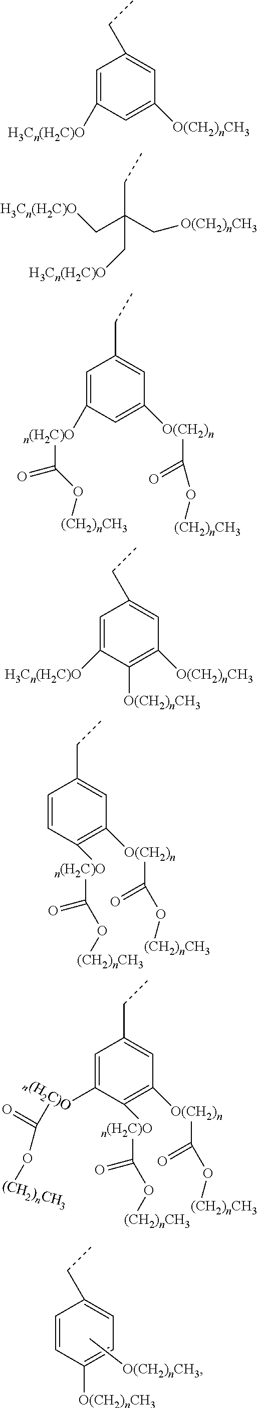

or any combination thereof.

In some embodiments, each occurrence of Y is independently selected from

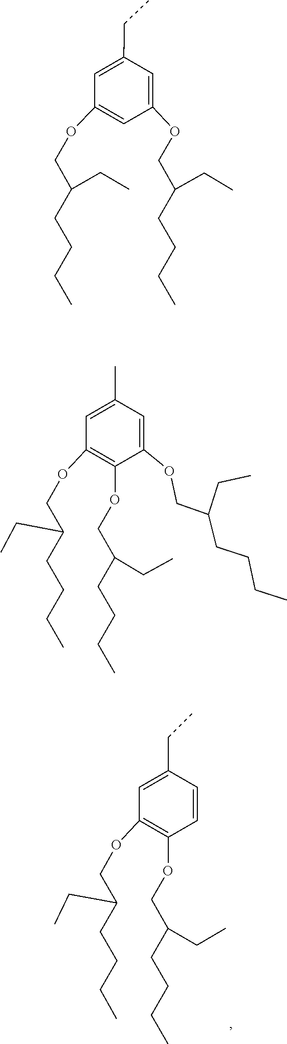

or any combination thereof.

In some embodiments, dashed lines indicate the connection to A.

In some embodiments, each occurrence of n is independently an integer from 1 to 20.

In some embodiments, each occurrence of —(CH₂)ₙCH₃, —O(CH₂)ₙ—, and —O(CH₂)ₙCH₃ is independently linear or branched.

In some embodiments, t is an integer from 2 to 5. Thus, in some embodiments, the amphiphilic Janus dendrimer comprises a first Y and a second Y. In some embodiments, the first Y comprises an alkyl chain having a different number of carbon atoms than the second Y comprises an alkyl chain. In some embodiments, the first Y comprises an alkyl chain having an even number of carbon atoms, and the second Y comprises an alkyl chain having an odd number of carbon atoms. In some embodiments, the ratio between the carbon atoms in the first Y and the carbon atoms in the second Y is greater than or equal to 3 and less than 7.

In one embodiment, u is 1 or 2. In one embodiment, u is 1. In one embodiment, u is 2.

In one embodiment, Z does not comprise an amine.

In one embodiment, Z is not a lipophilic group.

In one embodiment, Z comprises at least one carbohydrate.

In one embodiment, each occurrence of Z is independently selected from the following structures:

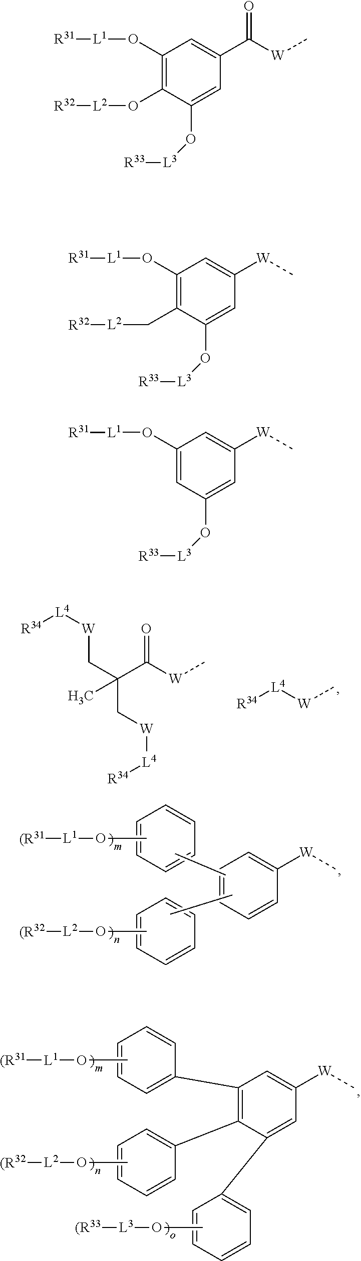

or any combination thereof.

In some embodiments, dashed lines indicate the connection to A.

In some embodiments, each occurrence of L¹, L², L³, and L⁴ is independently a covalent bond or a divalent linking group selected from alkylene, cycloalkylene, heteroalkylene, heterocycloalkylene, alkenylene, alkynylene, arylene, heteroarylene, silyl, amine, amide, ester, ether, carbonyl, carbamate, thioether, thioester, disulfide, hydrazine, urea, thiourea, phosphate, poly(alkyl ether), heteroatom, or any combination thereof. In one embodiment, at least one occurrence of L¹, L², L³, and L⁴ in the group Z represents a single bond. In one embodiment, at least one of L¹, L², L³, and L⁴ represents a group of formula —W—C(O)(CH₂)ₚC(O)—W—; wherein p is an integer between 1 and 10 and W represents NH, O, or S. In one embodiment, at least one of L¹, L², L³, and L⁴ comprises polyethylene glycol.

In some embodiments, each occurrence of R³¹, R³², R³³, and R³⁴ is independently selected from hydrogen, deuterium, alkyl, aryl, heteroaryl, cycloalkyl, alkoxy, phenoxy, amine, heterocycloalkyl, carbonyl, or any combination thereof.

In some embodiments, each occurrence of m, n, o is independently an integer from 1 to 5.

In one embodiment, each occurrence of Z is independently selected from the following structures:

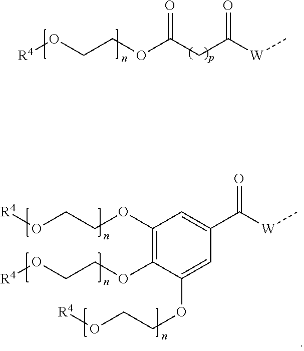

In some embodiments, each occurrence of n is independently an integer from 1 to 100.

In some embodiments, each occurrence of p is independently an integer from 1 to 10.

In some embodiments, each occurrence of R⁴ is independently selected from hydrogen, deuterium, alkyl, aryl, or any combination thereof.

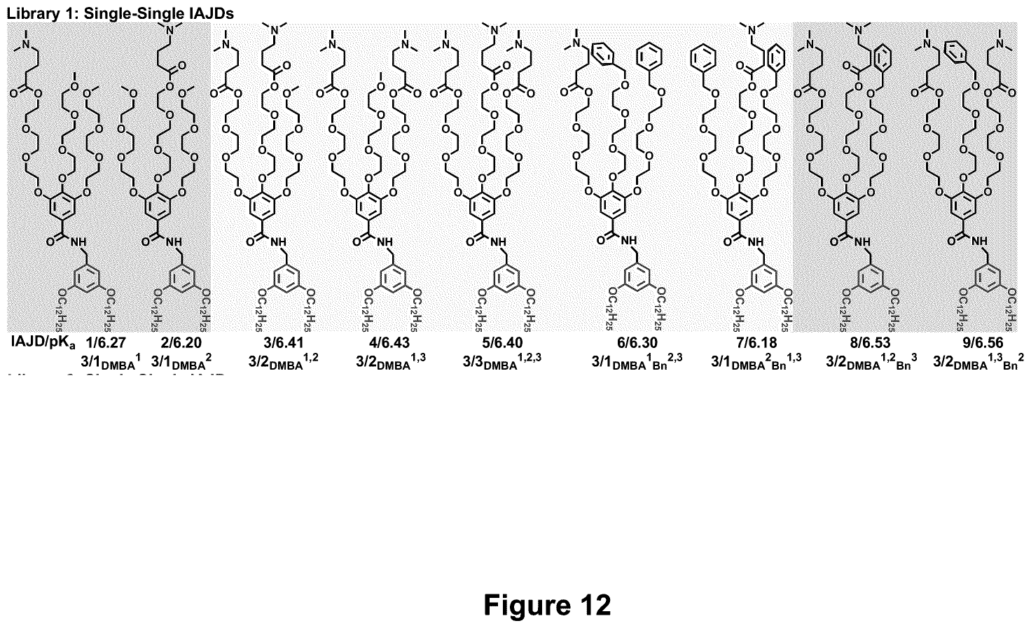

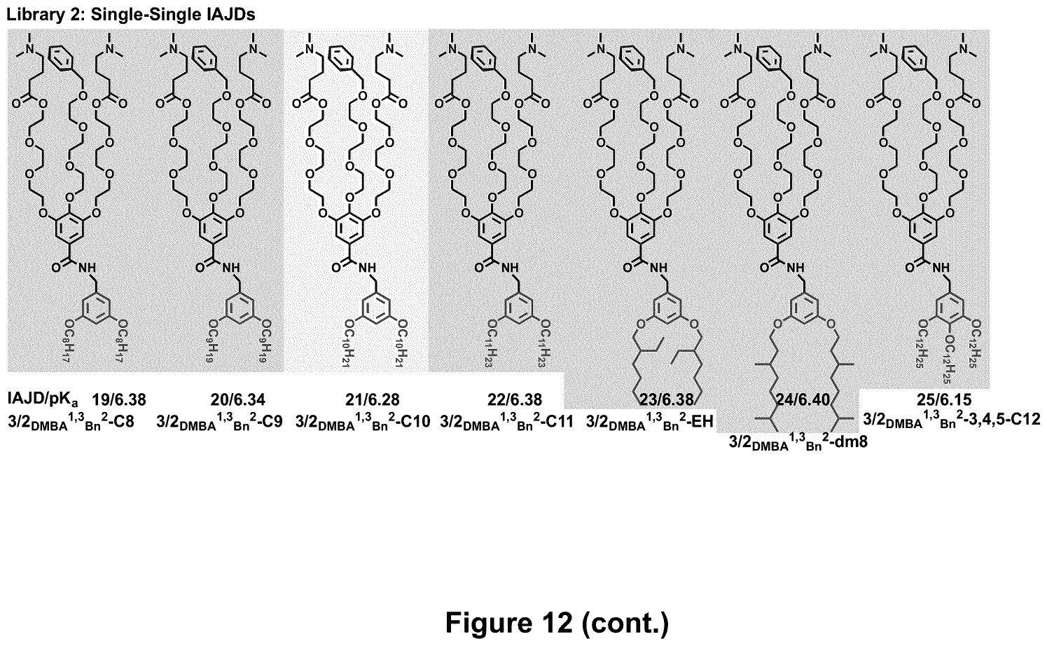

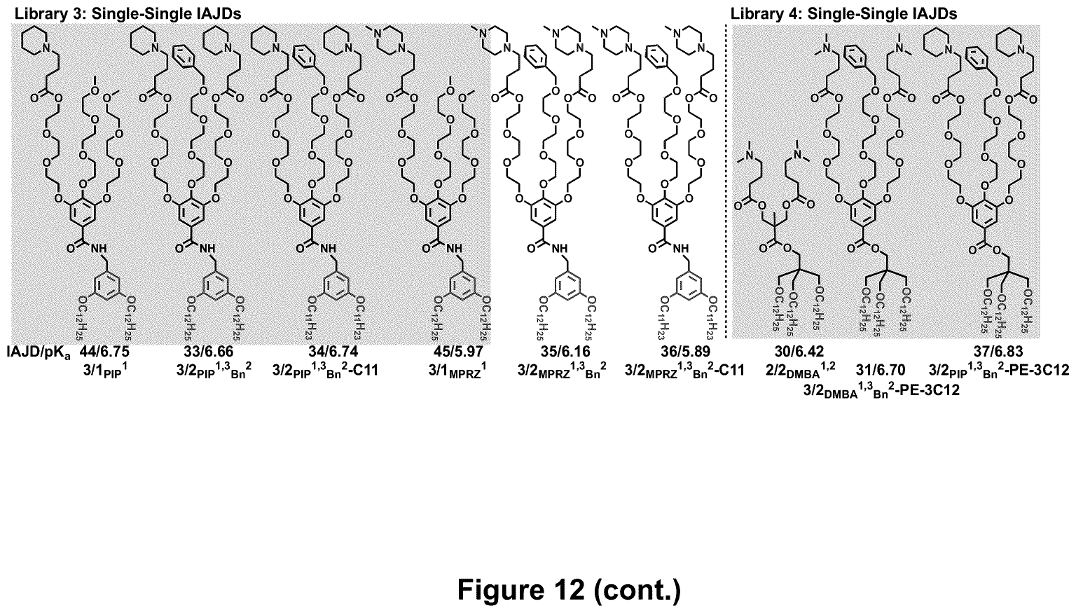

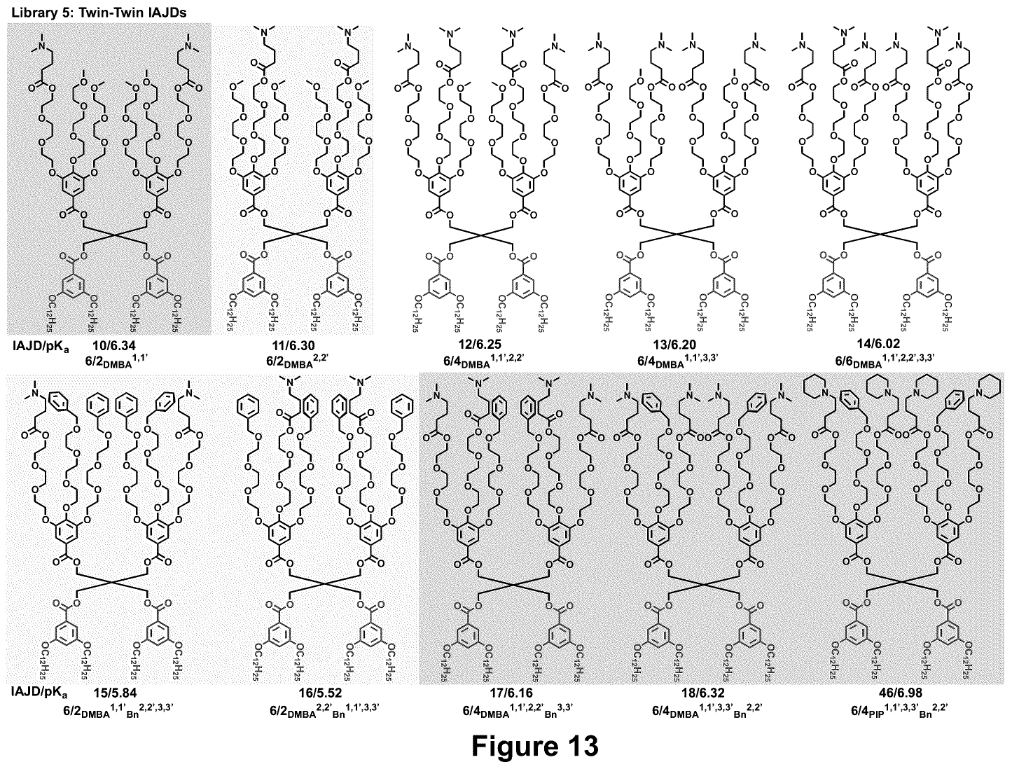

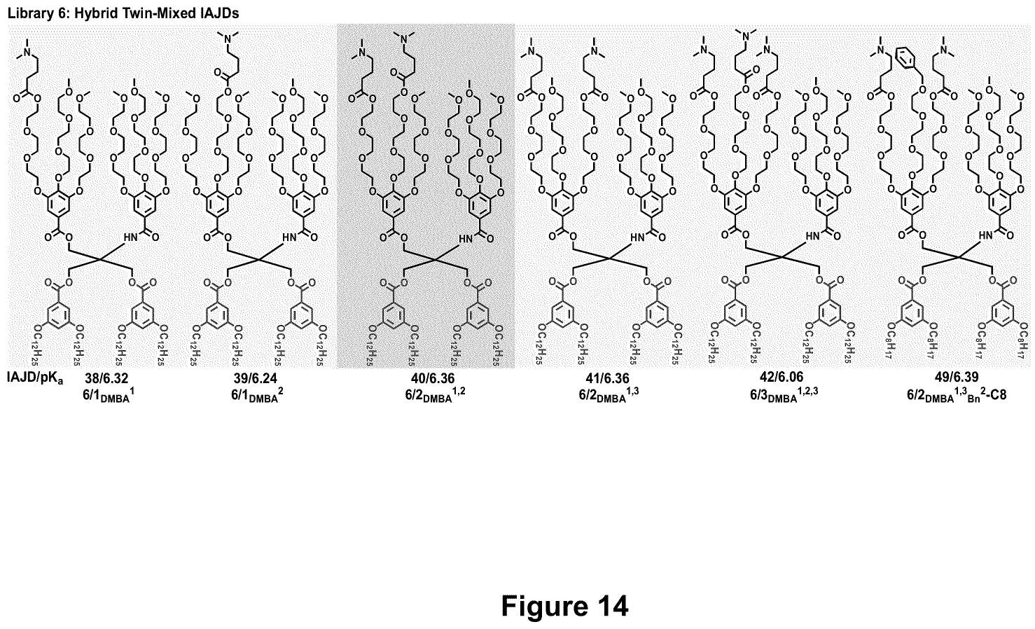

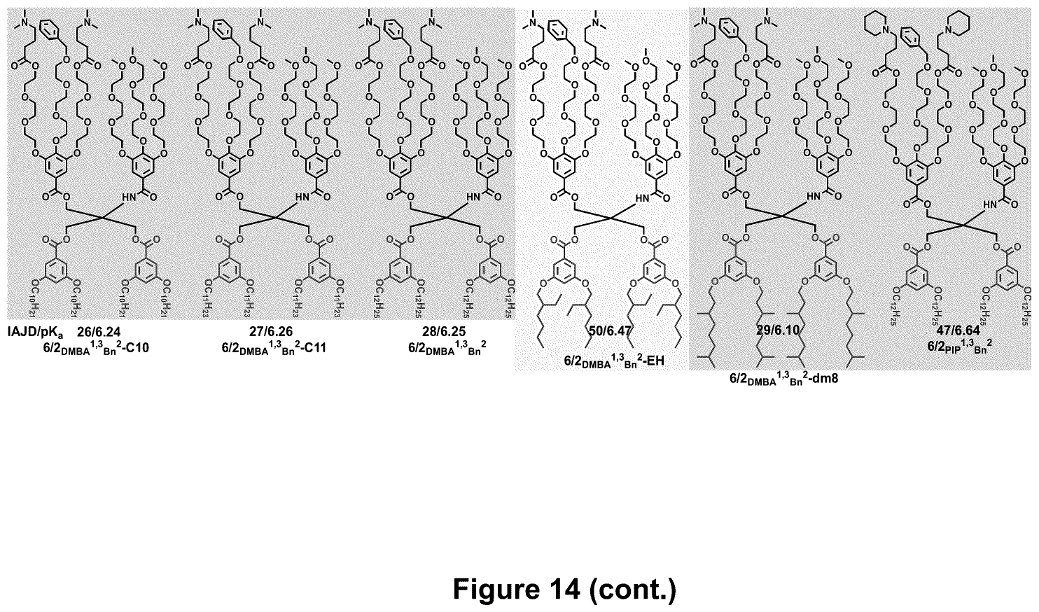

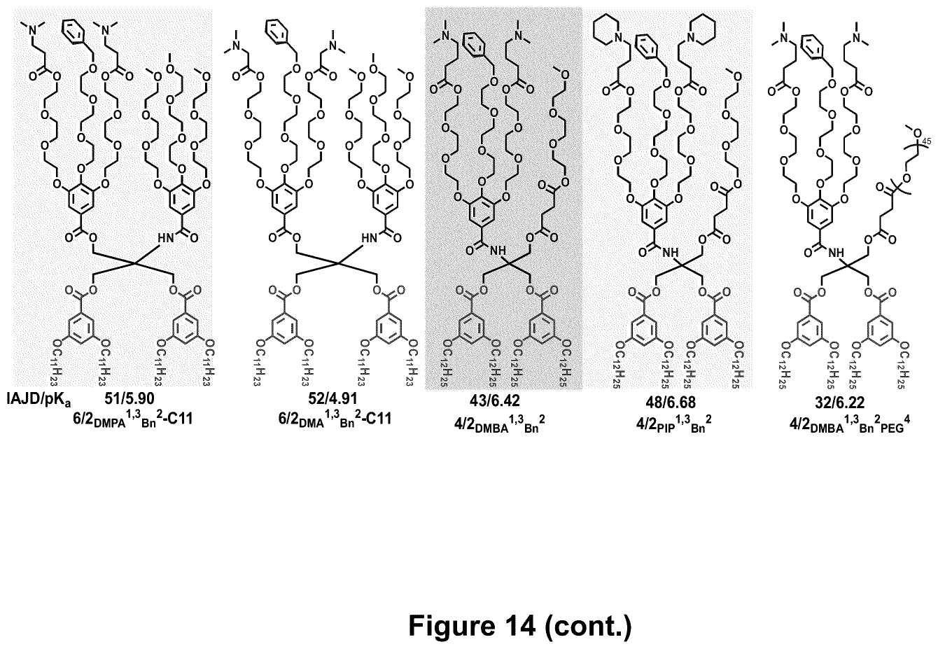

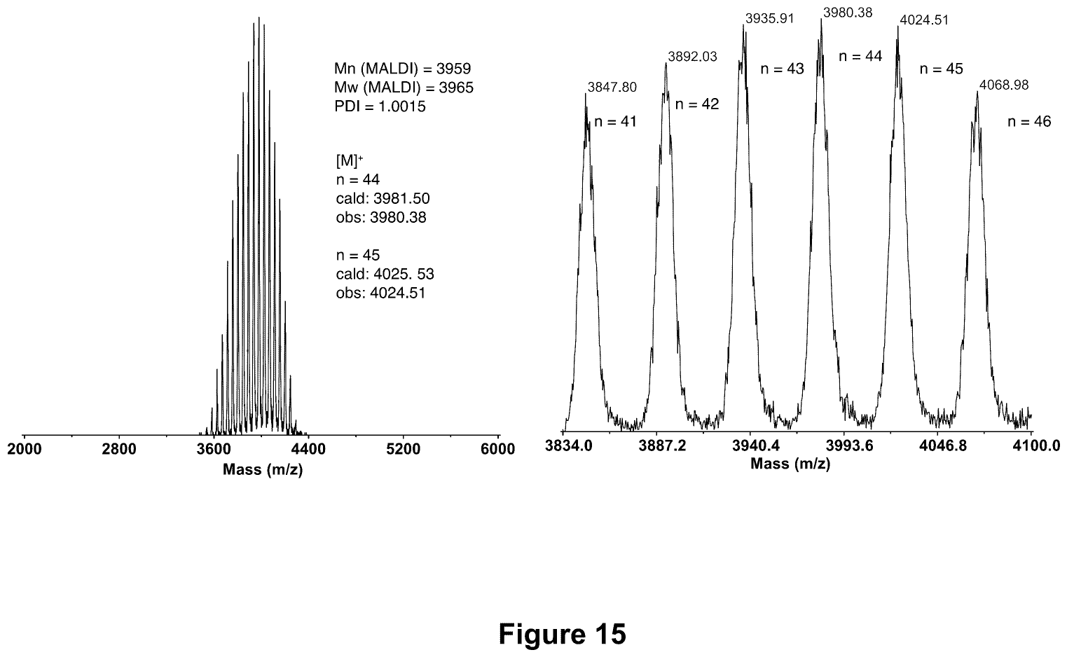

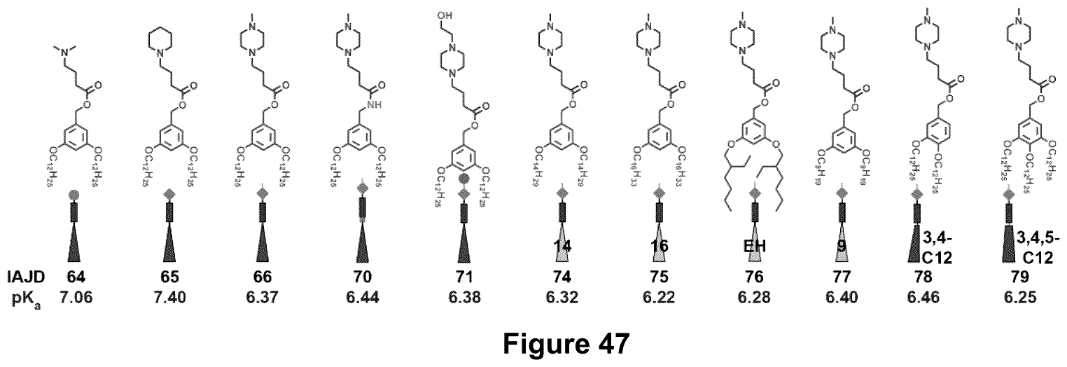

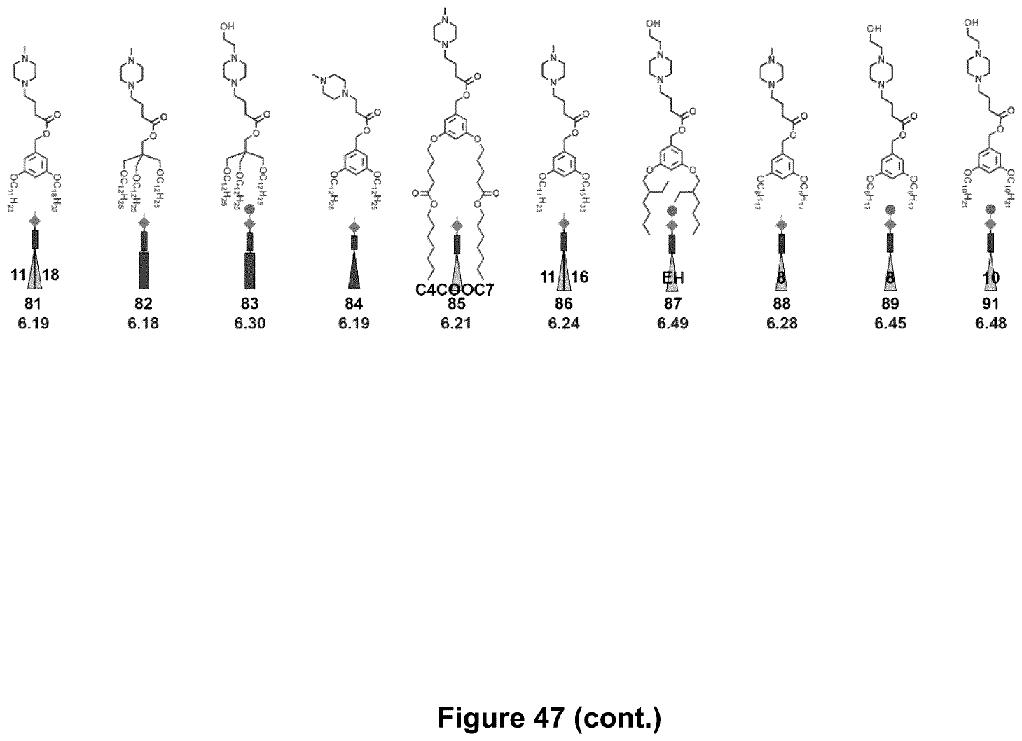

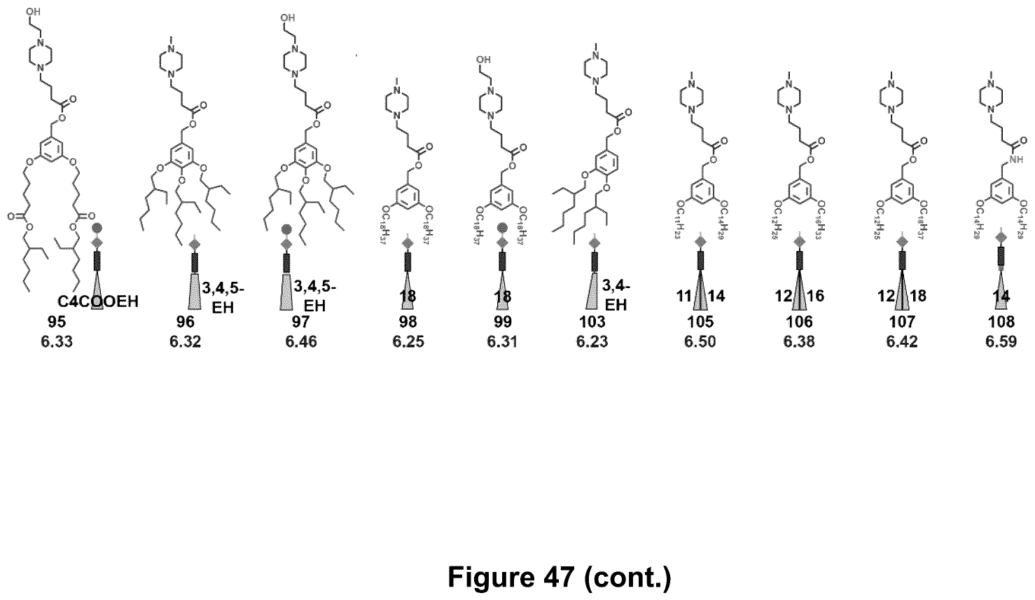

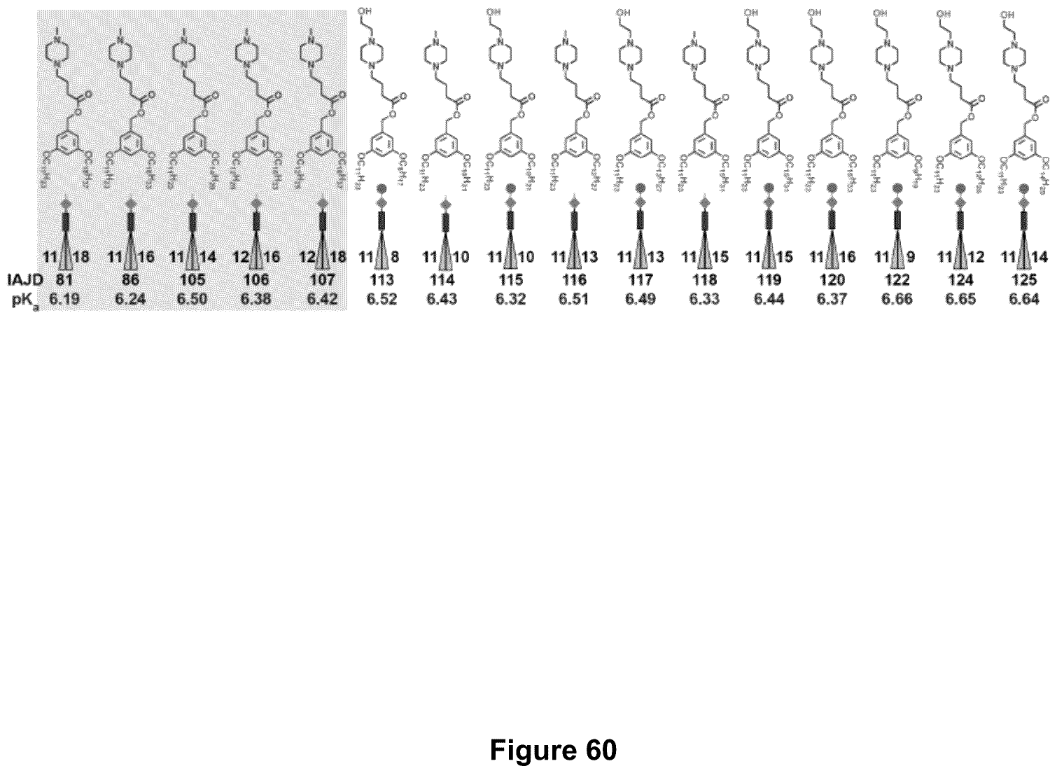

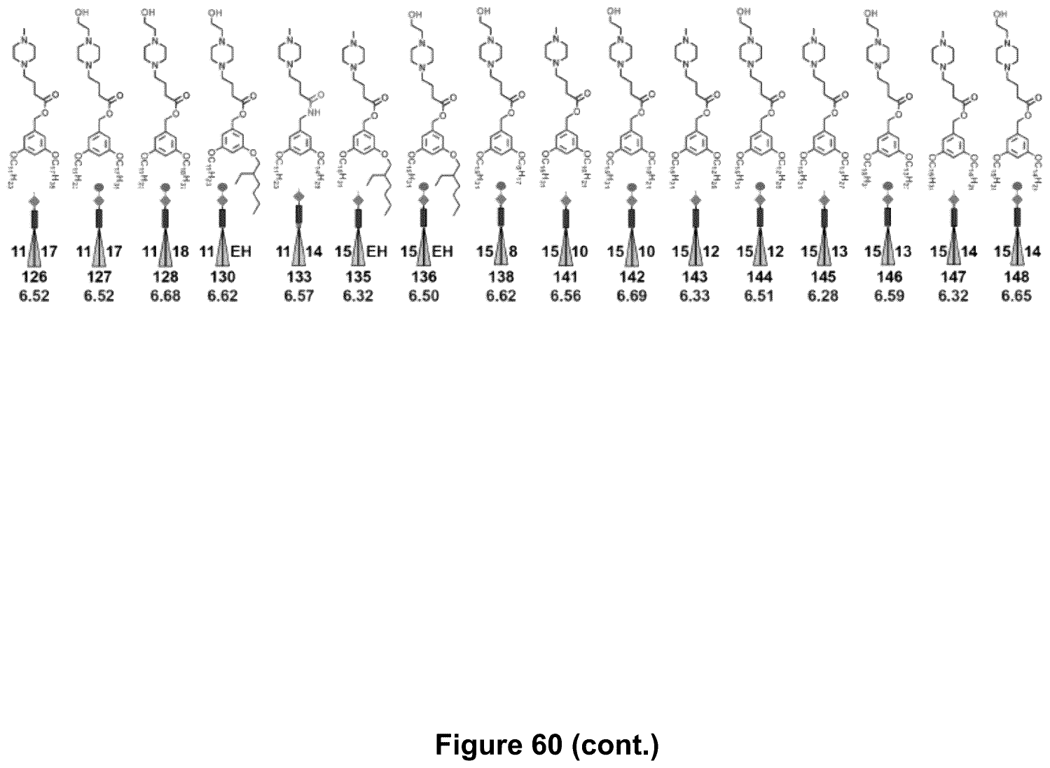

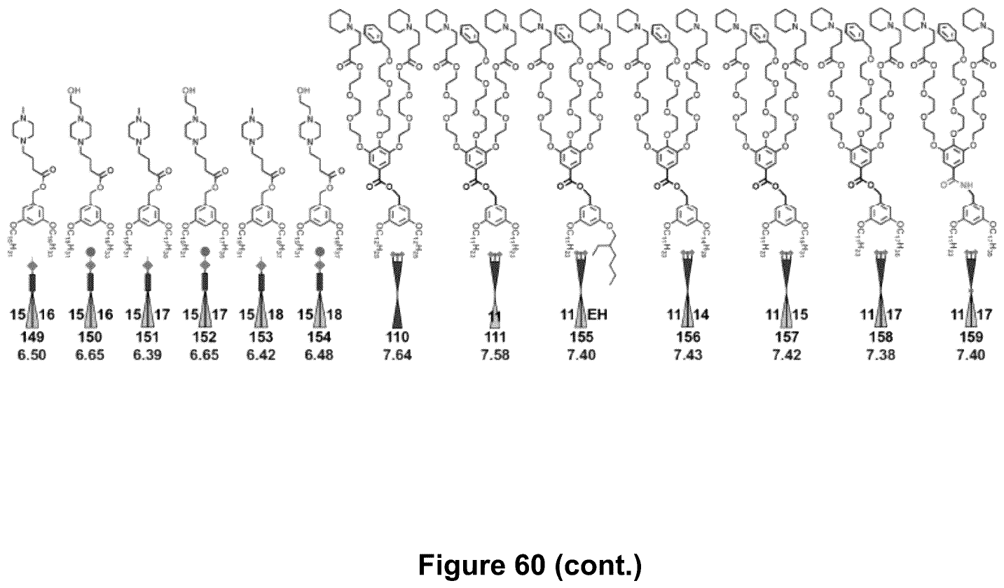

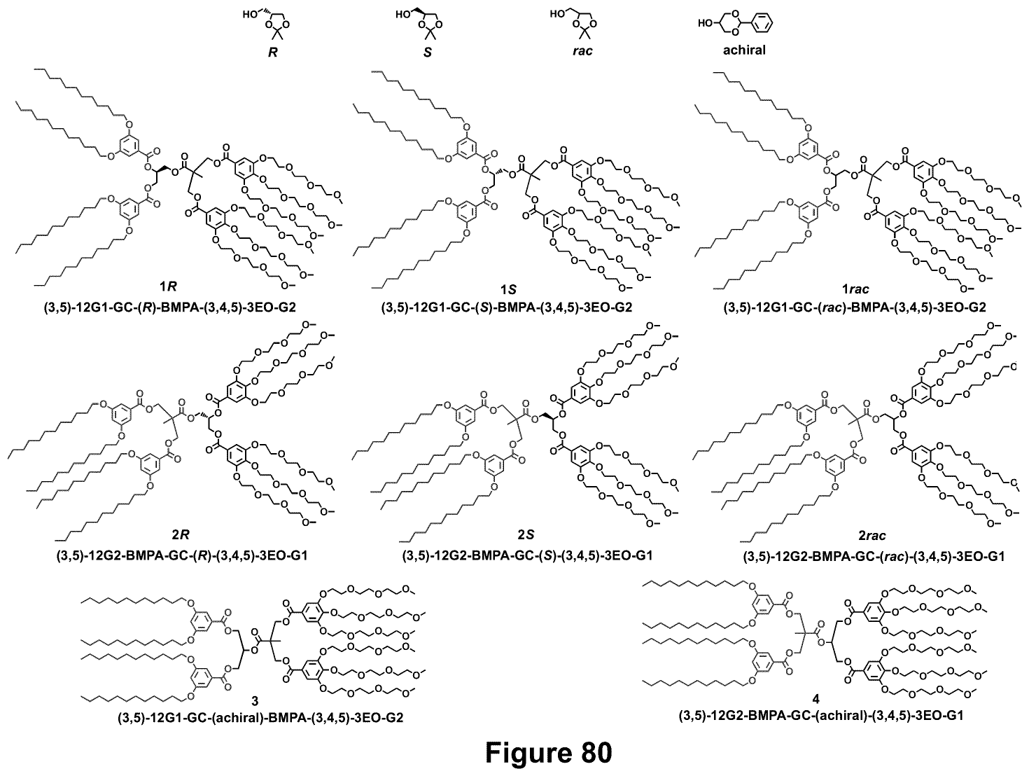

In some embodiments, the amphiphilic Janus dendrimer is an amphiphilic Janus dendrimer having a structure selected from at least one structure of FIG. 12, at least one structure of FIG. 13, at least one structure of FIG. 14, at least one structure of FIG. 47, at least one structure of FIG. 60, at least one structure of FIG. 80, or any combination thereof.

In some embodiments, the amphiphilic Janus dendrimer is an amphiphilic Janus dendrimer having a structure selected from:

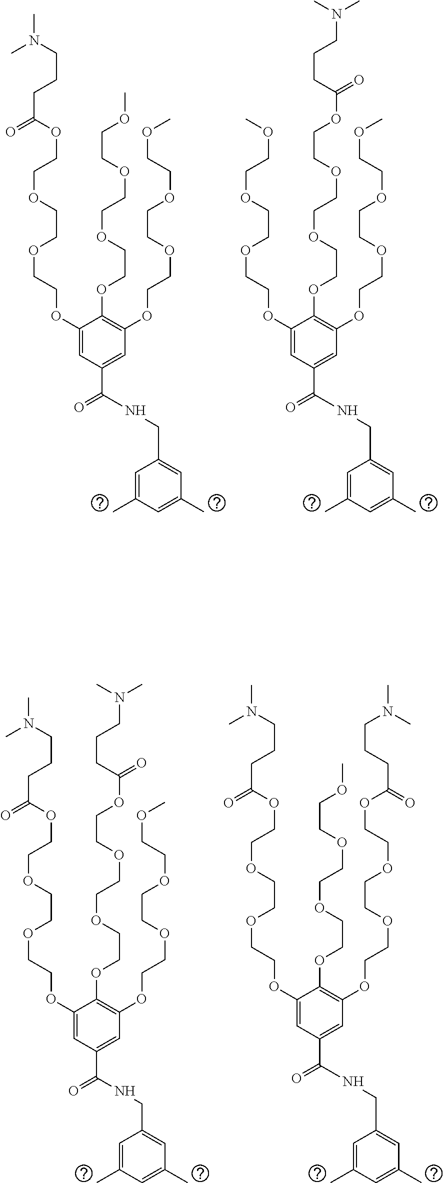 [SMILES: COCCOCCOCCOc1cc(C(=O)NCc2cc(C)cc(C)c2)cc(OCCOCCOCCOC(=O)CCCN(C)C)c1OCCOCCOCCOC ; COCCOCCOCCOc1cc(C(=O)NCc2cc(C)cc(C)c2)cc(OCCOCCOCCOC)c1OCCOCCOCCOC(=O)CCCN(C)C ; COCCOCCOCCOc1cc(C(=O)NCc2cc(C)cc(C)c2)cc(OCCOCCOCCOC(=O)CCCN(C)C)c1OCCOCCOCCOC(=O)CCCN(C)C ; COCCOCCOCCOc1c(OCCOCCOCCOC(=O)CCCN(C)C)cc(C(=O)NCc2cc(C)cc(C)c2)cc1OCCOCCOCCOC(=O)CCCN(C)C]

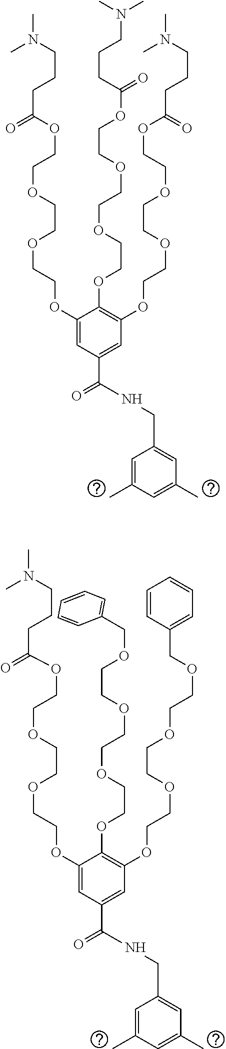 [SMILES: Cc1cc(C)cc(CNC(=O)c2cc(OCCOCCOCCOC(=O)CCCN(C)C)c(OCCOCCOCCOC(=O)CCCN(C)C)c(OCCOCCOCCOC(=O)CCCN(C)C)c2)c1 ; Cc1cc(C)cc(CNC(=O)c2cc(OCCOCCOCCOCc3ccccc3)c(OCCOCCOCCOCc3ccccc3)c(OCCOCCOCCOC(=O)CCCN(C)C)c2)c1]

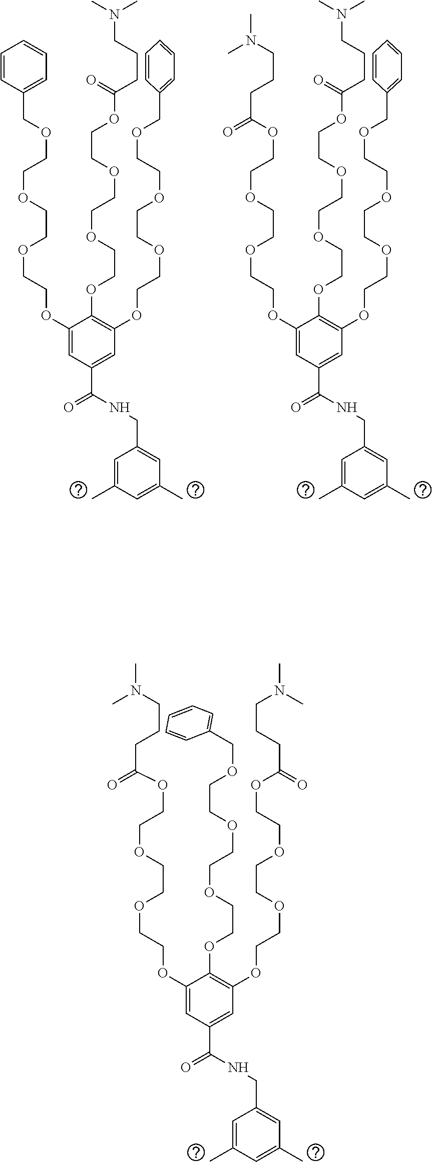 [SMILES: Cc1cc(C)cc(CNC(=O)c2cc(OCCOCCOCCOCc3ccccc3)c(OCCOCCOCCOC(=O)CCCN(C)C)c(OCCOCCOCCOCc3ccccc3)c2)c1 ; Cc1cc(C)cc(CNC(=O)c2cc(OCCOCCOCCOCc3ccccc3)c(OCCOCCOCCOC(=O)CCCN(C)C)c(OCCOCCOCCOC(=O)CCCN(C)C)c2)c1 ; Cc1cc(C)cc(CNC(=O)c2cc(OCCOCCOCCOC(=O)CCCN(C)C)c(OCCOCCOCCOCc3ccccc3)c(OCCOCCOCCOC(=O)CCCN(C)C)c2)c1]

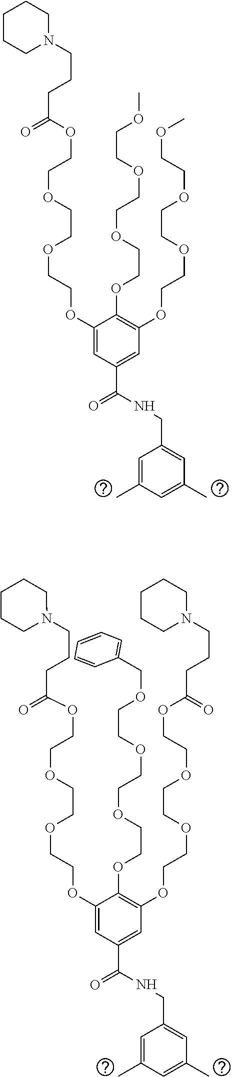 [SMILES: COCCOCCOCCOc1cc(C(=O)NCc2cc(C)cc(C)c2)cc(OCCOCCOCCOC(=O)CCCN2CCCCC2)c1OCCOCCOCCOC ; Cc1cc(C)cc(CNC(=O)c2cc(OCCOCCOCCOC(=O)CCCN3CCCCC3)c(OCCOCCOCCOCc3ccccc3)c(OCCOCCOCCOC(=O)CCCN3CCCCC3)c2)c1]

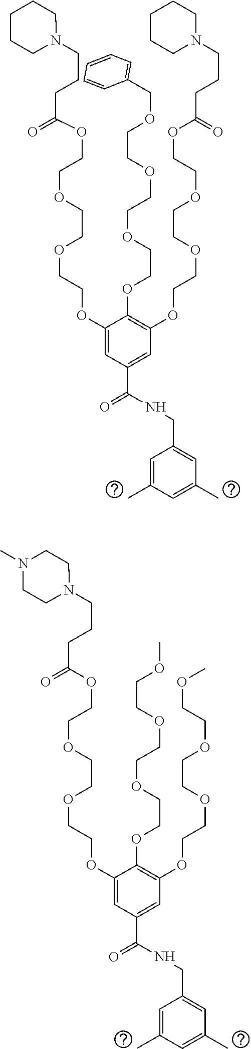

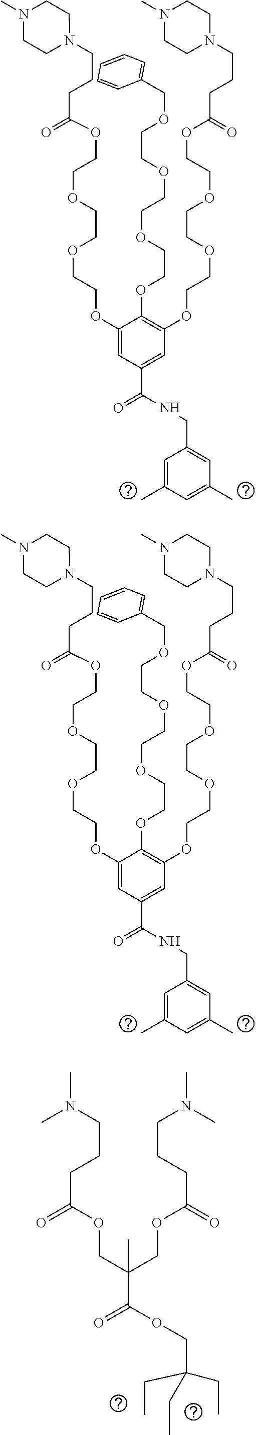

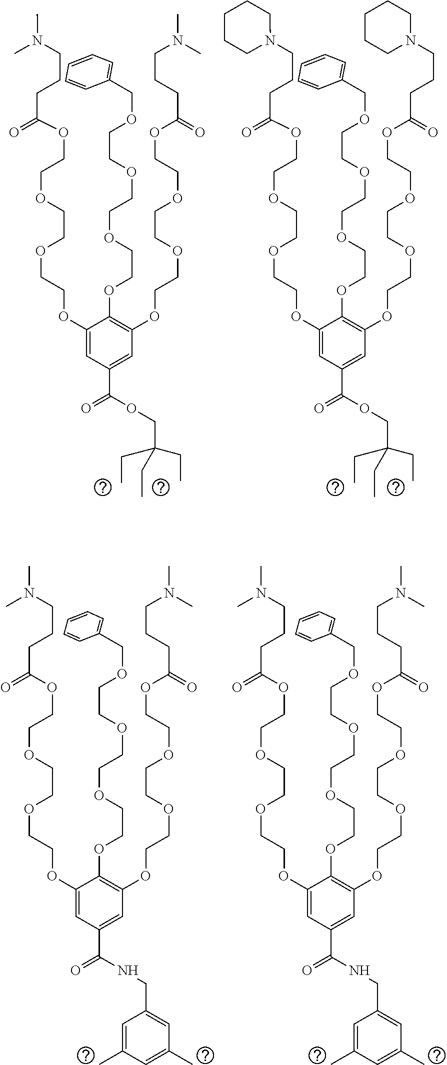

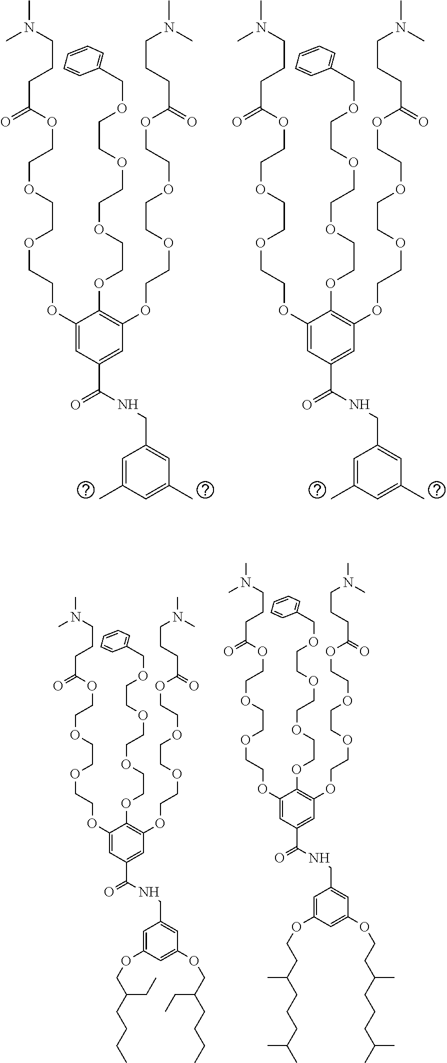 [SMILES: Cc1cc(C)cc(CNC(=O)c2cc(OCCOCCOCCOC(=O)CCCN(C)C)c(OCCOCCOCCOCc3ccccc3)c(OCCOCCOCCOC(=O)CCCN(C)C)c2)c1 ; Cc1cc(C)cc(CNC(=O)c2cc(OCCOCCOCCOC(=O)CCCN(C)C)c(OCCOCCOCCOCc3ccccc3)c(OCCOCCOCCOC(=O)CCCN(C)C)c2)c1 ; CCCCC(CC)COc1cc(CNC(=O)c2cc(OCCOCCOCCOC(=O)CCCN(C)C)c(OCCOCCOCCOCc3ccccc3)c(OCCOCCOCCOC(=O)CCCN(C)C)c2)cc(OCC(CC)CCCC)c1 ; CC(C)CCCC(C)CCOc1cc(CNC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCC(C)CCCC(C)C)c1]

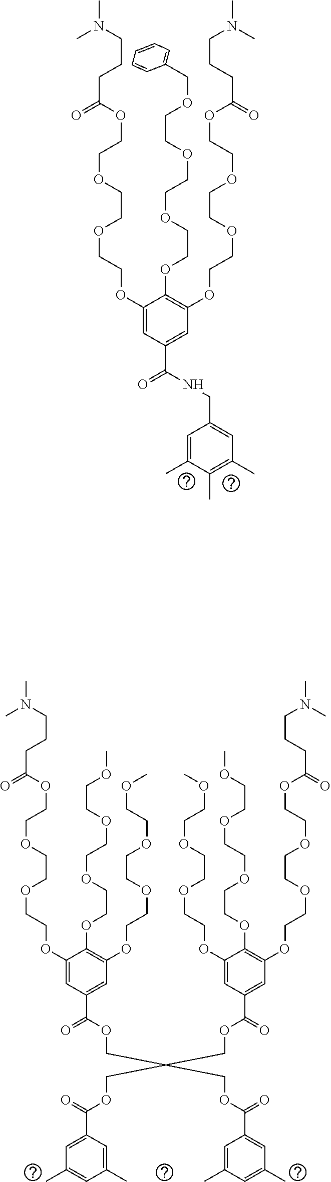 [SMILES: Cc1cc(CNC(=O)c2cc(OCCOCCOCCOC(=O)CCCN(C)C)c(OCCOCCOCCOCc3ccccc3)c(OCCOCCOCCOC(=O)CCCN(C)C)c2)cc(C)c1C ; COCCOCCOCCOc1cc(C(=O)OCC(COC(=O)c2cc(C)cc(C)c2)(COC(=O)c2cc(C)cc(C)c2)COC(=O)c2cc(OCCOCCOCCOC)c(OCCOCCOCCOC)c(OCCOCCOCCOC(=O)CCCN(C)C)c2)cc(OCCOCCOCCOC(=O)CCCN(C)C)c1OCCOCCOCCOC]

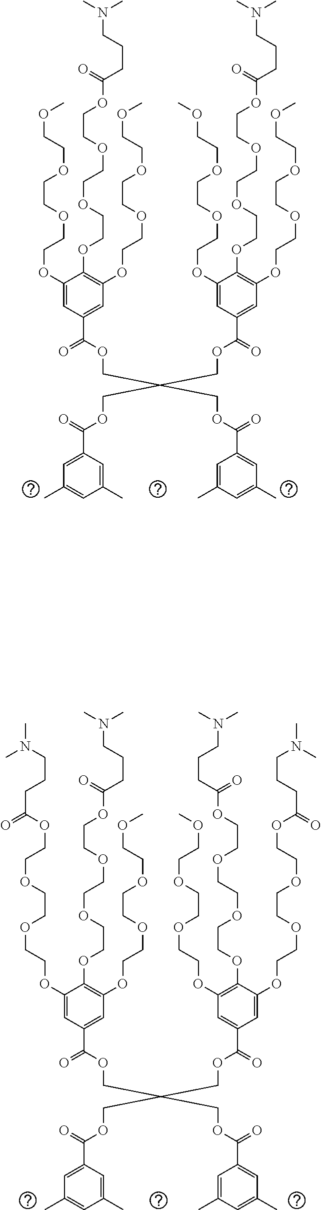 [SMILES: COCCOCCOCCOc1cc(C(=O)OCC(COC(=O)c2cc(C)cc(C)c2)(COC(=O)c2cc(C)cc(C)c2)COC(=O)c2cc(OCCOCCOCCOC)c(OCCOCCOCCOC(=O)CCCN(C)C)c(OCCOCCOCCOC)c2)cc(OCCOCCOCCOC)c1OCCOCCOCCOC(=O)CCCN(C)C ; COCCOCCOCCOc1cc(C(=O)OCC(COC(=O)c2cc(C)cc(C)c2)(COC(=O)c2cc(C)cc(C)c2)COC(=O)c2cc(OCCOCCOCCOC)c(OCCOCCOCCOC(=O)CCCN(C)C)c(OCCOCCOCCOC(=O)CCCN(C)C)c2)cc(OCCOCCOCCOC(=O)CCCN(C)C)c1OCCOCCOCCOC(=O)CCCN(C)C]

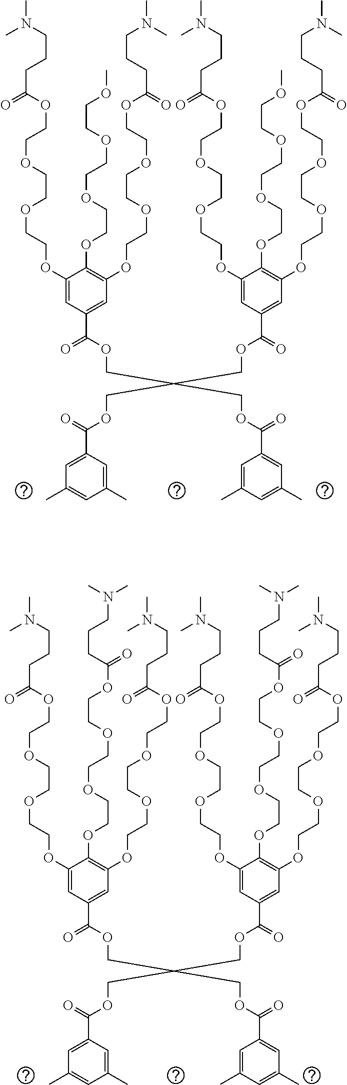 [SMILES: COCCOCCOCCOc1c(OCCOCCOCCOC(=O)CCCN(C)C)cc(C(=O)OCC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOC)c(OCCOCCOCCOC(=O)CCCN(C)C)c2)cc1OCCOCCOCCOC(=O)CCCN(C)C ; Cc1cc(C)cc(C(=O)OCC(COC(=O)c2cc(C)cc(C)c2)(COC(=O)c2cc(OCCOCCOCCOC(=O)CCCN(C)C)c(OCCOCCOCCOC(=O)CCCN(C)C)c(OCCOCCOCCOC(=O)CCCN(C)C)c2)COC(=O)c2cc(OCCOCCOCCOC(=O)CCCN(C)C)c(OCCOCCOCCOC(=O)CCCN(C)C)c(OCCOCCOCCOC(=O)CCCN(C)C)c2)c1]

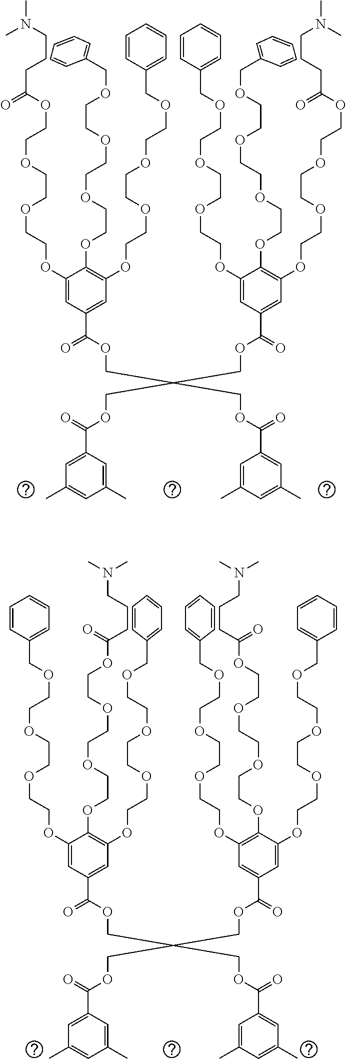 [SMILES: Cc1cc(C)cc(C(=O)OCC(COC(=O)c2cc(C)cc(C)c2)(COC(=O)c2cc(OCCOCCOCCOCc3ccccc3)c(OCCOCCOCCOCc3ccccc3)c(OCCOCCOCCOC(=O)CCCN(C)C)c2)COC(=O)c2cc(OCCOCCOCCOCc3ccccc3)c(OCCOCCOCCOCc3ccccc3)c(OCCOCCOCCOC(=O)CCCN(C)C)c2)c1 ; Cc1cc(C)cc(C(=O)OCC(COC(=O)c2cc(C)cc(C)c2)(COC(=O)c2cc(OCCOCCOCCOCc3ccccc3)c(OCCOCCOCCOC(=O)CCCN(C)C)c(OCCOCCOCCOCc3ccccc3)c2)COC(=O)c2cc(OCCOCCOCCOCc3ccccc3)c(OCCOCCOCCOC(=O)CCCN(C)C)c(OCCOCCOCCOCc3ccccc3)c2)c1]

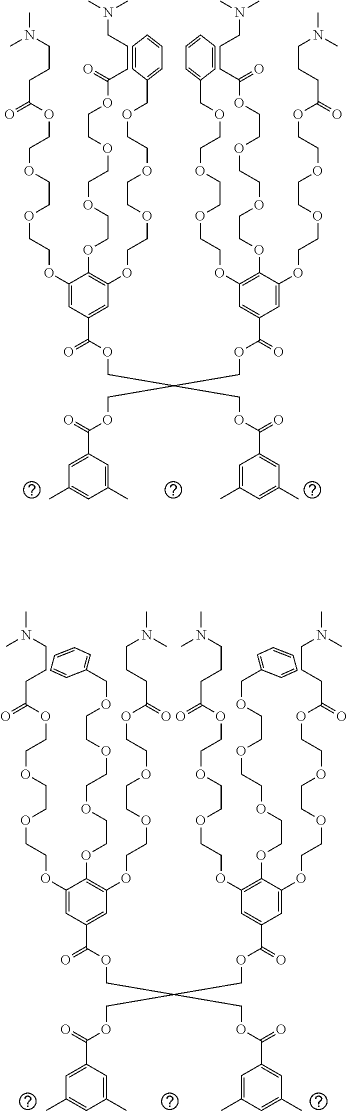 [SMILES: Cc1cc(C)cc(C(=O)OCC(COC(=O)c2cc(C)cc(C)c2)(COC(=O)c2cc(OCCOCCOCCOCc3ccccc3)c(OCCOCCOCCOC(=O)CCCN(C)C)c(OCCOCCOCCOC(=O)CCCN(C)C)c2)COC(=O)c2cc(OCCOCCOCCOCc3ccccc3)c(OCCOCCOCCOC(=O)CCCN(C)C)c(OCCOCCOCCOC(=O)CCCN(C)C)c2)c1 ; Cc1cc(C)cc(C(=O)OCC(COC(=O)c2cc(C)cc(C)c2)(COC(=O)c2cc(OCCOCCOCCOC(=O)CCCN(C)C)c(OCCOCCOCCOCc3ccccc3)c(OCCOCCOCCOC(=O)CCCN(C)C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)c1]

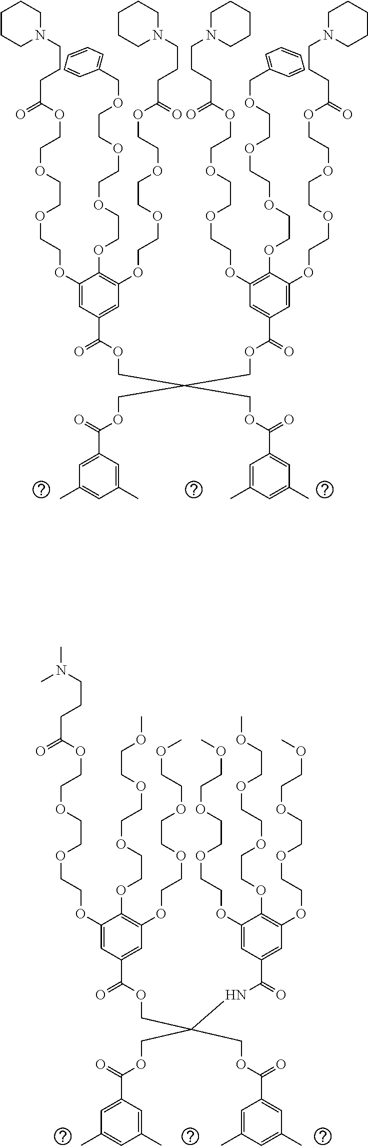 [SMILES: Cc1cc(C)cc(C(=O)OCC(COC(=O)c2cc(C)cc(C)c2)(COC(=O)c2cc(OCCOCCOCCOC(=O)CCCN3CCCCC3)c(OCCOCCOCCOCc3ccccc3)c(OCCOCCOCCOC(=O)CCCN3CCCCC3)c2)COC(=O)c2cc(OCCOCCOCCOC(=O)CCCN3CCCCC3)c(OCCOCCOCCOCc3ccccc3)c(OCCOCCOCCOC(=O)CCCN3CCCCC3)c2)c1 ; COCCOCCOCCOc1cc(C(=O)NC(COC(=O)c2cc(C)cc(C)c2)(COC(=O)c2cc(C)cc(C)c2)COC(=O)c2cc(OCCOCCOCCOC)c(OCCOCCOCCOC)c(OCCOCCOCCOC(=O)CCCN(C)C)c2)cc(OCCOCCOCCOC)c1OCCOCCOCCOC]

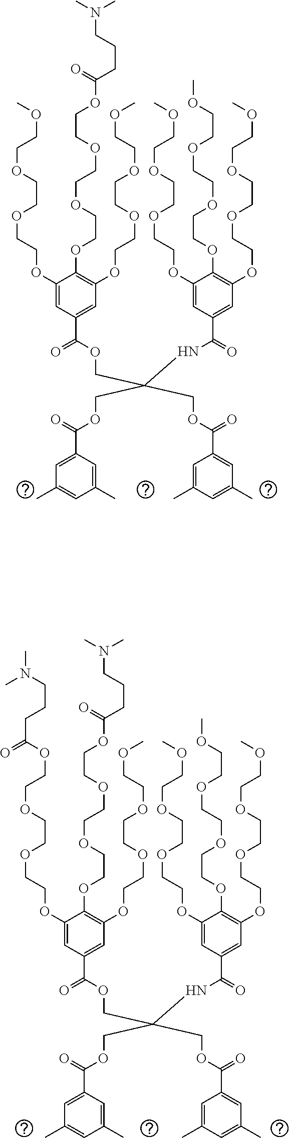 [SMILES: COCCOCCOCCOc1cc(C(=O)NC(COC(=O)c2cc(C)cc(C)c2)(COC(=O)c2cc(C)cc(C)c2)COC(=O)c2cc(OCCOCCOCCOC)c(OCCOCCOCCOC(=O)CCCN(C)C)c(OCCOCCOCCOC)c2)cc(OCCOCCOCCOC)c1OCCOCCOCCOC ; COCCOCCOCCOc1cc(C(=O)NC(COC(=O)c2cc(C)cc(C)c2)(COC(=O)c2cc(C)cc(C)c2)COC(=O)c2cc(OCCOCCOCCOC)c(OCCOCCOCCOC(=O)CCCN(C)C)c(OCCOCCOCCOC(=O)CCCN(C)C)c2)cc(OCCOCCOCCOC)c1OCCOCCOCCOC]

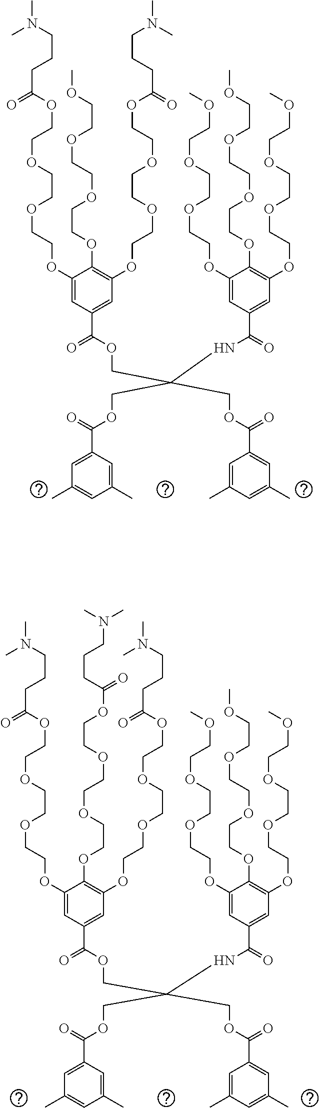 [SMILES: COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOC)c(OCCOCCOCCOC(=O)CCCN(C)C)c2)cc(OCCOCCOCCOC)c1OCCOCCOCCOC ; COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOC(=O)CCCN(C)C)c(OCCOCCOCCOC(=O)CCCN(C)C)c2)cc(OCCOCCOCCOC)c1OCCOCCOCCOC]

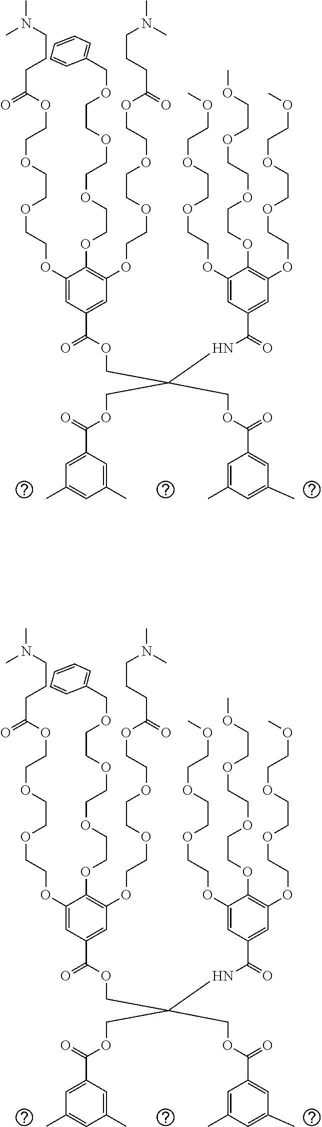 [SMILES: COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCOCCOCCOC)c1OCCOCCOCCOC ; COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCOCCOCCOC)c1OCCOCCOCCOC]

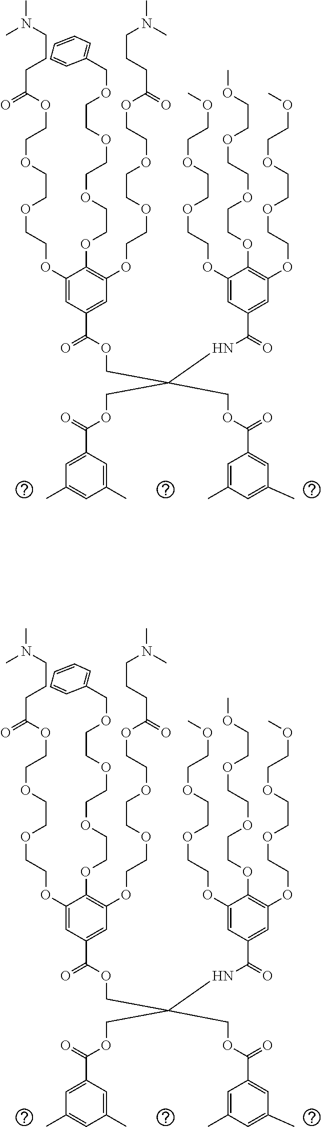 [SMILES: COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCOCCOCCOC)c1OCCOCCOCCOC ; COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCOCCOCCOC)c1OCCOCCOCCOC]

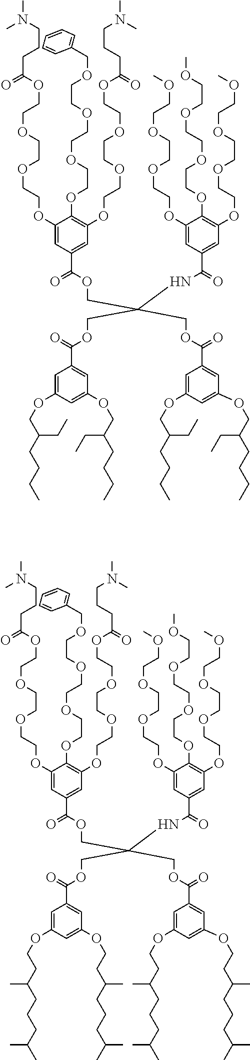 [SMILES: CCCCC(CC)COc1cc(OCC(CC)CCCC)cc(C(=O)OCC(COC(=O)c2cc(OCC(CC)CCCC)cc(OCC(CC)CCCC)c2)(COC(=O)c2cc(OCCOCCOCCOC(=O)CCCN(C)C)c(OCCOCCOCCOCc3ccccc3)c(OCCOCCOCCOC(=O)CCCN(C)C)c2)NC(=O)c2cc(OCCOCCOCCOC)c(OCCOCCOCCOC)c(OCCOCCOCCOC)c2)c1 ; COCCOCCOCCOc1cc(C(=O)NC(COC(=O)c2cc(OCCC(C)CCCC(C)C)cc(OCCC(C)CCCC(C)C)c2)(COC(=O)c2cc(OCCC(C)CCCC(C)C)cc(OCCC(C)CCCC(C)C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCOCCOCCOC)c1OCCOCCOCCOC]

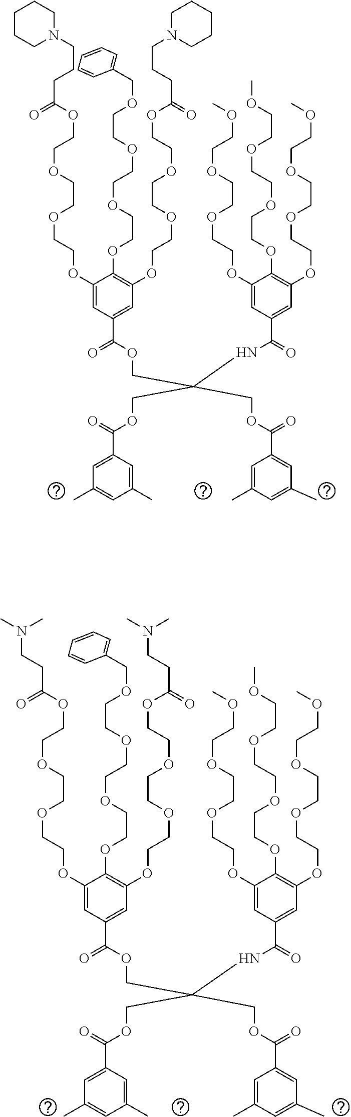

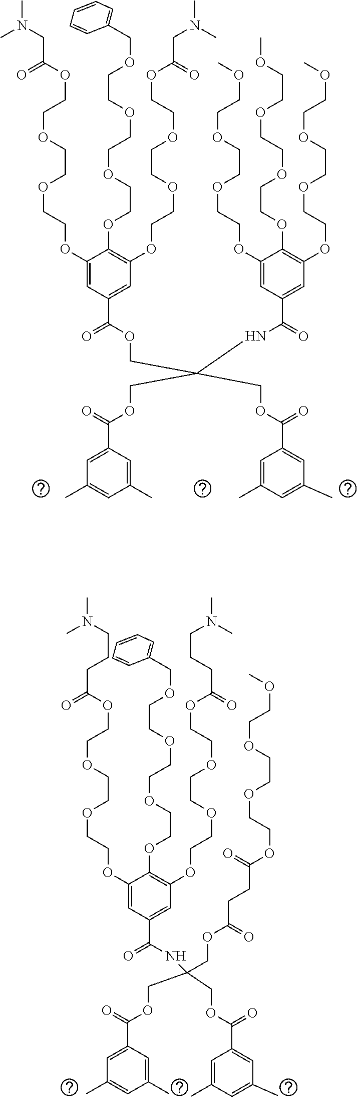

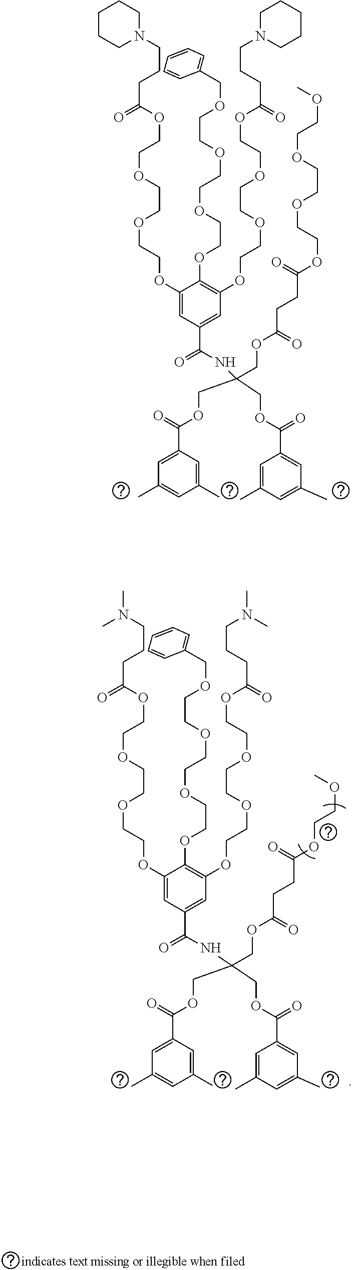

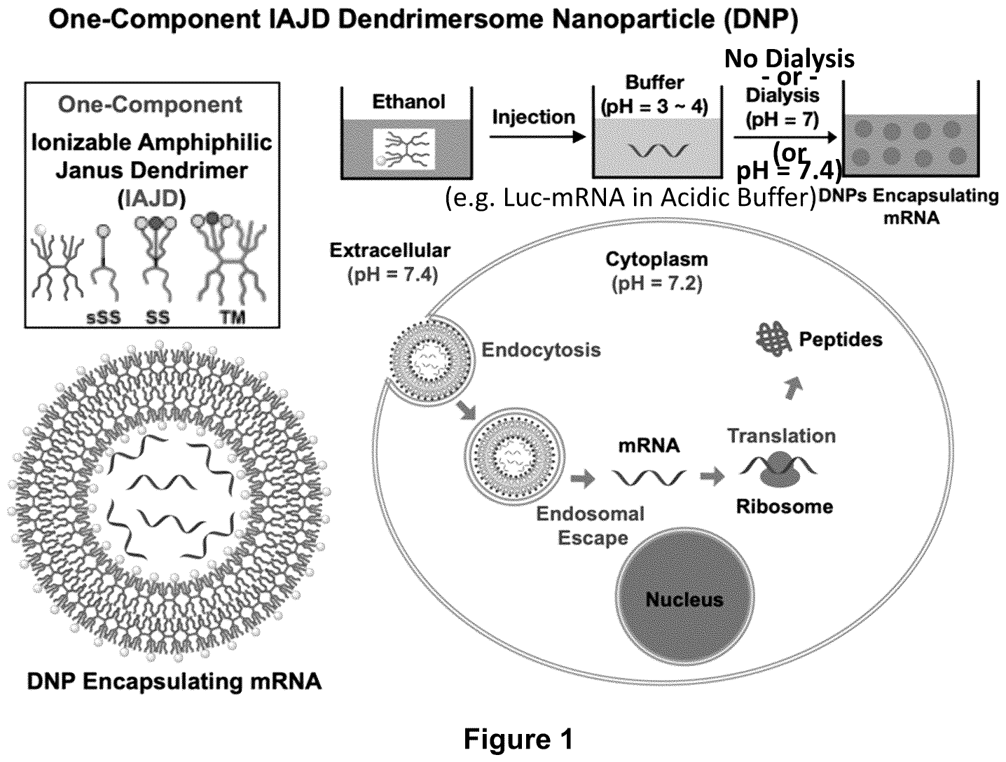

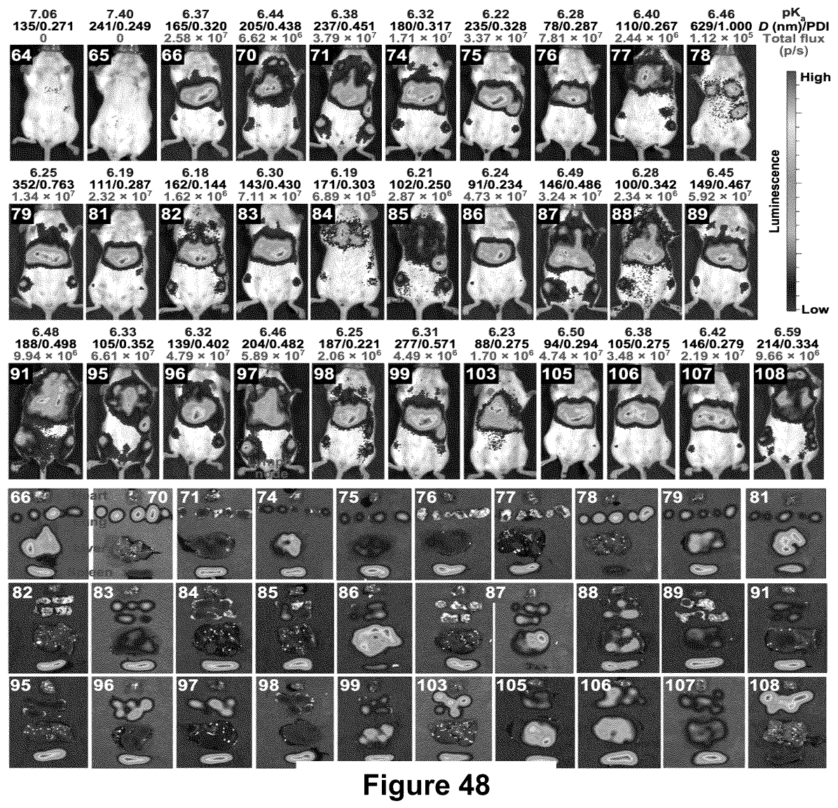

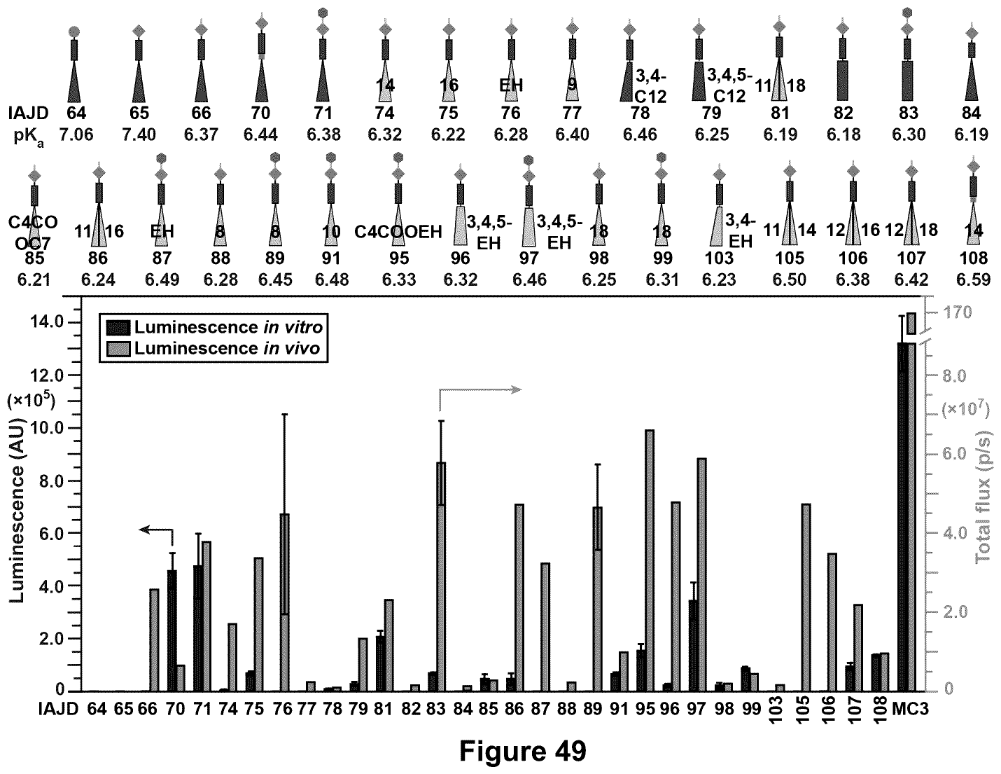

In some embodiments, amphiphilic Janus dendrimer having the structure of Formula (II) comprise an amine (the ionizable amine) in X that is sufficient to provide the dual hydrophilic and binding role in the IAJD. For example, FIG. 47 summarizes IAJDs as described herein, synthesized, and investigated in vitro and in vivo, their schematic representations and the pKₐ values. pKₐ values were determined as described in the examples. Co-assembly of all IAJDs from FIG. 47 with Luc-mRNA was performed by the injection of an ethanol solution of IAJD into an acetate buffer of pH=4. The acetate buffer contained mRNA added to the buffer from neutral water. Further details are found in the Example section below. The co-assembly forms a nanoparticle (DNP). Co-assembly is shown schematically in FIG. 1. FIG. 48 shows in vivo transfection results of the DNPs co-assembled from the IAJDs, shown in FIG. 1. The IAJD numbers are shown on the top left, IAJD pKₐ values, size in nm, with the polydispersities (PDI) of the resulting DNPs, are shown above each mouse image. The scale of the luminescence values is also shown. Representative images of mRNA delivery to different organs are shown in the bottom part of this figure. FIG. 49 shows a comparison of the activity of DNPs assembled from the IAJDs, shown schematically in the top part of the Figure, in vitro (in blue) and in vivo (in red). Thus, in some embodiments, in dendrimers having the structure of Formula (II), the valency of A may be two, and the sum of s and t may be two.

In some embodiments, the amphiphilic Janus dendrimer comprises a homochiral, racemic, or achiral branding points. Thus, in some embodiments, the ionizable amphiphilic Janus dendrimer is a homochiral ionizable amphiphilic Janus dendrimer, racemic ionizable amphiphilic Janus dendrimer, or achiral ionizable amphiphilic Janus dendrimer.

In some embodiments, the amphiphilic Janus dendrimer is a symmetric amphiphilic Janus dendrimer.

In some embodiments, the amphiphilic Janus dendrimer is an asymmetric amphiphilic Janus dendrimer.

In some embodiments, the amphiphilic Janus dendrimer is a nonsymmetric amphiphilic Janus dendrimer.

For example, in some embodiments, the successful design of the hydrophobic region of the amphiphilic Janus dendrimer is based, in part, on the usage of dissimilar alkyl lengths and discovery of the unexpectedly important role of the primary structure of the hydrophobic part of the IAJDs which increases the activity of targeted delivery of a desired cargo to a target site up to 90.2 fold. In some embodiments, the nonsymmetric one-component amphiphilic Janus dendrimers do not require microfluidic or T-tube technology employed by 4-component LNPs to co-assemble with mRNA. In some embodiments, the one-component systems may co-assemble with mRNA into DNPs with about 97% nucleic acid encapsulation efficiency by simple injection of their ethanol solution into an acidic buffer containing mRNA rather than by microfluidic or T-tube technology required by LNPs.

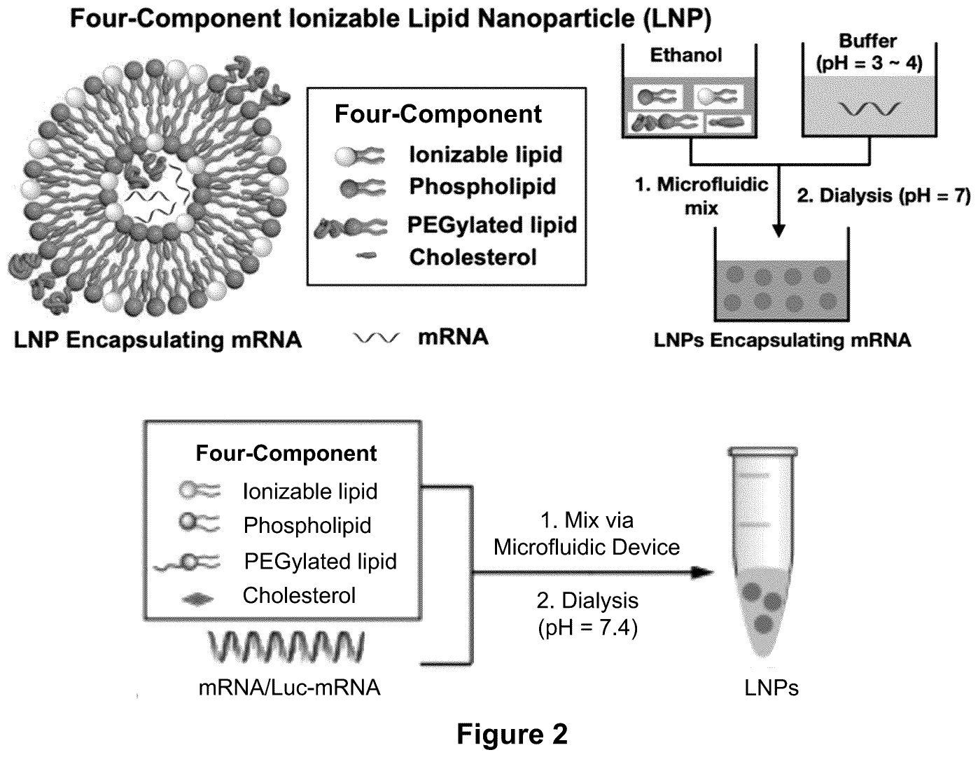

For example, FIG. 2 outlines the structure and co-assembly with mRNA of LNPs. FIG. 1 shows the structures of sSS, SS and TM IAJDs with dissimilar and similar alkyl groups in their hydrophobic parts and their co-assembly with mRNA. IAJDs can be described as single-single, (SS, single hydrophilic dendron connected to single lipophilic dendron), twin-twin (TT, two hydrophilic dendrons connected to two lipophilic dendrons) and twin-mixed (TM, two different hydrophilic dendrons connected to two lipophilic dendrons) IAJDs.

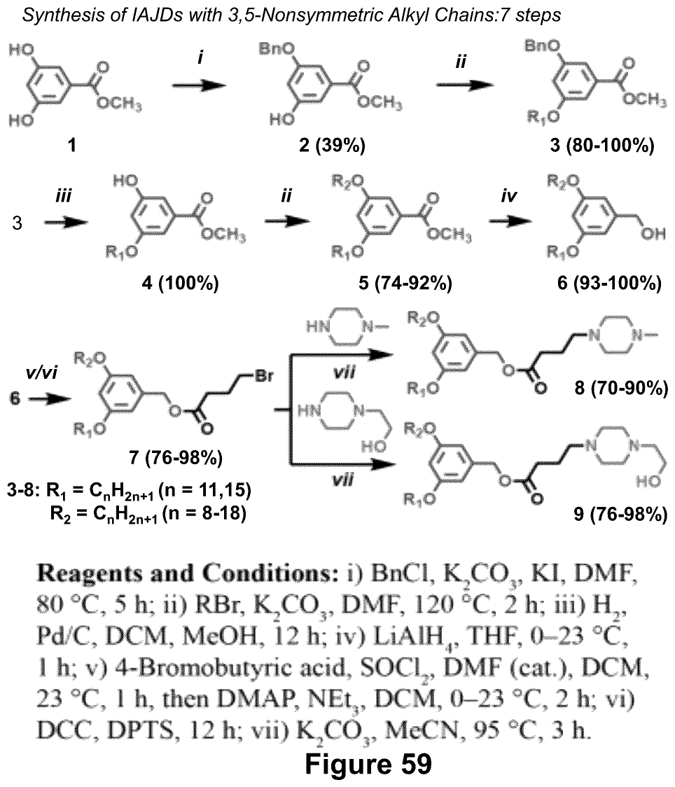

For example, FIG. 59 shows the synthesis of nonsymmetric IAJDs. In the first step the 3-benzylether of 3,5-dihydroxy methyl benzoate was produced in 39% isolated yield in 5 hours by etherification of 1 with BnCl at 80° C. in DMF. Subsequently 2 was alkylated with 1-bromoundecane or 1bromopentadecane in DMF, with K₂CO₃ base at 120° C. to produce 80-100% isolated yield of 3. Hydrogenolysis of 3 (H₂/Pd, DCM/MeOH, 12 hours) produced 4 in 100% isolated yield. Alkylation of 4 with varying alkyl lengths from 1-bromooctane to 1-bromooctadecane in DMF, with K₂CO₃ base at 120° C. generated the nonsymmetric compounds 5 in 74-92% isolated yield. Reduction of compounds 5 with LiAlH₄ in THF (0-23° C., 1 hour) produced the benzyl alcohols 6 in 93-100% isolated yield. Compounds 6 were reacted with 4-bromobutyric acid either via its acid chloride generated with SOCl₂ catalyzed by DMF in CH₂Cl₂ at 23° C. followed by esterification in the presence of NEt₃/DMAP (0-23° C., 2 hours) or by direct esterification with DCC/DPTS in 12 hours to produce compounds 7 in 76 to 98% isolated yield. Compounds 7 were reacted with methylpiperazine or hydroxyethyl piperazine (K₂CO₃, MeCN, 95° C., 3 hours) to yield IAJDs 8 (70-90% isolated yield) and 9 (76-98% isolated yield). Their purity was determined by a combination of HPLC, MALDI-TOF and NMR and was higher than 99%. Their structures are shown in FIG. 60 (IAJDs 113 to 178). IAJD133 has a similar structure with IAJD105 except that the interconnecting ester group of 105 was replaced with an amide in 133. The benzyl amine precursor of 133 was generated from the corresponding benzyl alcohol via its benzyl chloride obtained with SOCl₂ followed by reaction with K-phthalimide and subsequently hydrazine as reported.

Single-single (SS) IAJDs reported previously to display very high activity for delivery to lung were synthesized with nonsymmetric alkyl groups in their lipophilic part. They are IAJDs 110 to 159 from FIG. 60. Their sequence-defined hydrophilic dendrons were synthesized as reported in Zhang et al., J. Am. Chem. Soc. 2021, 143, 12315-12327. The hydrophilic dendrons were reacted with selected nonsymmetric lipophilic dendrons 6 or their amine. For convenience, the IAJDs are referred to by their number followed by the ratio between their two alkyl groups forming their nonsymmetric lipophilic part. This nomenclature together with their entire and schematic structures shown in FIG. 60 facilitates the discussion of their in vitro and in vivo activity vs molecular structure. For example, 116(11/13) and 117(11/13) both contain a combination of 11 and 13 carbons in their lipophilic part but 116 contains a methyl piperazine while 117 a hydroxyethyl piperazine ionizable amine. The large red dot on the top of the cartoon for 117 refers to hydroxyethyl while the blue thin-line on 116 indicates the methyl group, both attached to piperazine (FIG. 60). A combination of 33 IAJDs sSS with 7 IAJDs SS are shown in FIG. 60. IAJDs 81, 86, 105, 106 and 107 marked in blue on top left corner of FIG. 60 were reported previously (Zhang et al., J. Am. Chem. Soc. 2021, 143, 17975-17982). Transfection experiments with Luc-mRNA were performed both in vitro and in vivo by following the methodology reported in Zhang et al., J. Am. Chem. Soc. 2021, 143, 12315-12327 and Zhang et al., J. Am. Chem. Soc. 2021, 143, 17975-17982.

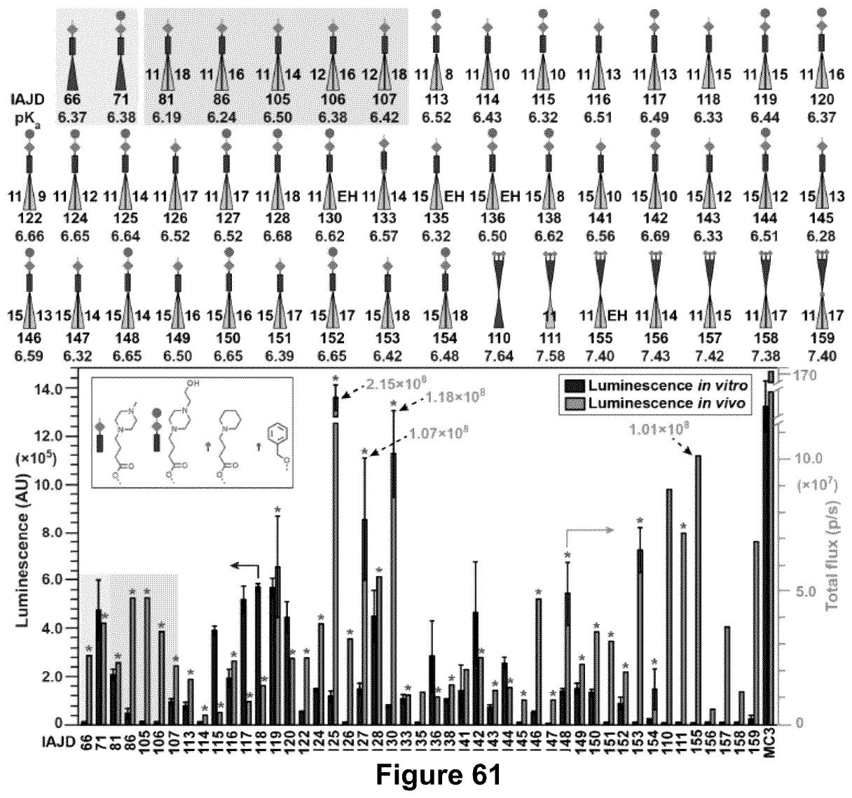

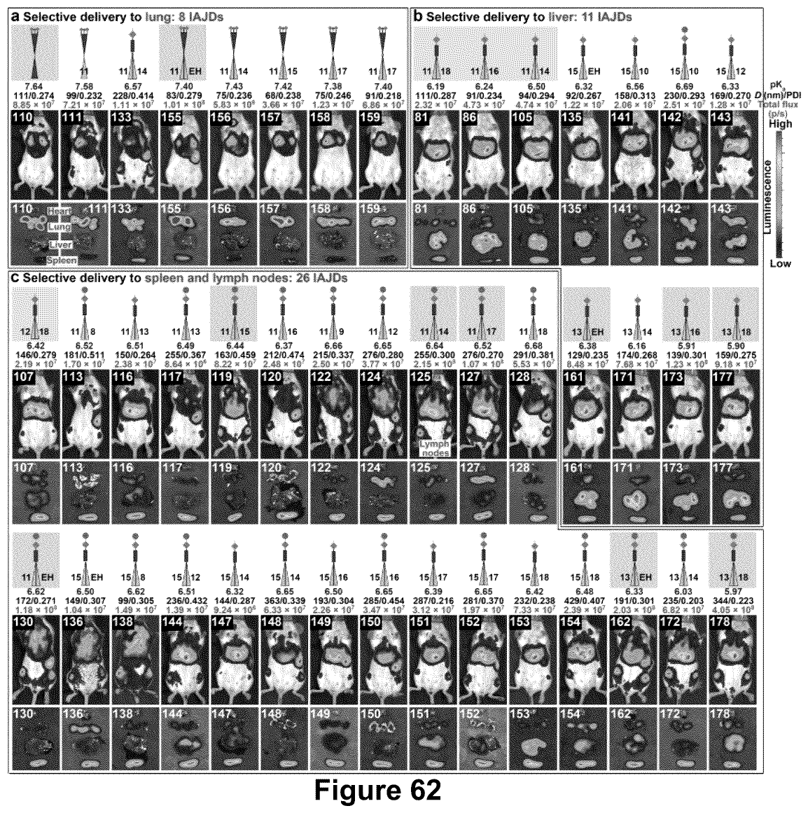

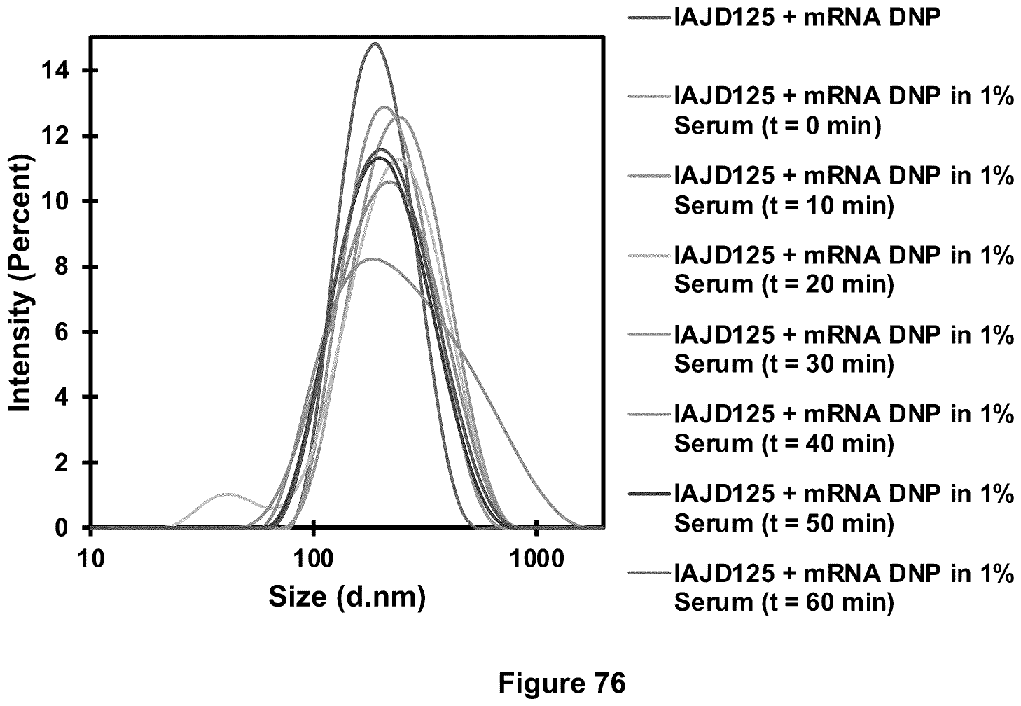

The overall transfection activity in vivo was analyzed according to its target selectivity and organized in FIG. 62. The first important result of the transfection experiments is that 11 IAJDs show approximately 108 activity, 2 for lung, 3 for liver and 6 for spleen and lymph (FIG. 62 marked in pink, FIG. 74). The symmetric IAJDs 110(12/12) and 111(11/11) were previously reported to show the highest activity to lung as IAJDs 33(12/12) and 34(11/11) when they contained an amide interconnecting group. The new IAJDs 110(12/12) and 111(11/11) containing an interconnecting ester rather than amide group show also very high activity to lung. Nonsymmetric IAJDs are stable in serum and PBS buffer and exhibit very high activity in lung by a mechanism different from aggregation (Tables shown below and FIG. 76 through FIG. 78). The highest activity of all IAJDs is for 178(13/18), which displays a total flux luminescence of 4.05×10⁸ p/s, that is 90.2 times higher than of symmetric 99 with the same headgroup (18/18) and only 4.2 times lower than of MC3 (FIG. 61). It is also important to mention that the transition from 158(11/17) to 159(11/17), the second an IAJD containing an amide interconnecting group, while the first an ester, increased activity about 6-fold. This demonstrates the significance of amide interconnecting groups for the delivery to lung but simultaneously reveals that the presence of oligooxyethylenes in the hydrophilic part may be important for targeted delivery to lung. These experiments demonstrate the important role of the dissimilar alkyl groups from the hydrophobic part of IAJDs.

Finally, FIG. 61 summarizes the activity of all IAJDs made in the examples and compares them with their symmetric (marked in light blue) and nonsymmetric (marked in blue) IAJDs which can be used as control experiments. The results from FIG. 61 show that an increase of up to 90.2 times in the activity of the IAJDs was observed by changing their primary structure in the lipophilic part from symmetric to nonsymmetric. Ratios between the two-alkyl length, preferably from odd-even combinations, may be equal or larger than 3 and less than 7 and seem to result in the largest increase in activity. Selected examples of IAJDs supporting this conclusion are provided by IAJDs 119(11/15), 125(11/14), 127(11/17), 128(11/18), 130(11/EH), 153(15/18), 155(11/EH), 159(11/17).

In some embodiments, the nonsymmetric amphiphilic Janus dendrimer is stable at around 5° C.

### Nanoparticles

In one aspect, the invention relates to nanoparticles comprising at least one amphiphilic Janus dendrimer of the present invention.

In various embodiments, the nanoparticle is a one-component nanoparticle.

In various embodiments, the nanoparticle is a four-component nanoparticle.

In some embodiments, the nanoparticle comprises a homochiral ionizable amphiphilic Janus dendrimer, achiral ionizable amphiphilic Janus dendrimer, or any combination thereof.

In some embodiments, the nanoparticle is a unilamellar nanoparticle.

In some embodiments, the nanoparticle is a multilamellar nanoparticle.

In some embodiments, the nanoparticle is an onion multilamellar nanoparticle.

In some embodiments, the nanoparticle is a racemic ionizable amphiphilic Janus dendrimer.

In some embodiments, the nanoparticle is a dendrimersome nanoparticle (DNP).

In some embodiments, the nanoparticle comprises at least two amphiphilic Janus dendrimers. Thus, in some embodiments, the the nanoparticle comprises a first ionizable amphiphilic Janus dendrimer and a second ionizable amphiphilic Janus dendrimer. In some embodiments, the first ionizable amphiphilic Janus dendrimer has a different structure than the second ionizable amphiphilic Janus dendrimer.

In some embodiments, the nanoparticles further comprise at least one amphiphilic Janus dendrimer disclosed in Wang, et al., J. Am. Chem. Soc. 2020, 142, 9525-9536; Xiao et al., J. Am. Chem. Soc. 2016, 138, 12655-12663; Torre et al., Proc. Natl. Acad. Sci. U.S.A. 2019, 116, 15378-15385; Percec et al., J. Am. Chem. Soc. 2021, 143, 17724-17743; Wilson et al., J. Polym. Sci. Part A: Polymer Chemistry, 2010, 2498-2508; Xiao et al., Proc. Natl. Acad. Sci. U.S.A 2017, E7045-E7053; and U.S. Patent Application Publication No. 2012277460; and U.S. Pat. No. 8,614,347; each of which is hereby incorporated by reference in their entireties.

In various embodiments, the nanoparticle has a mean diameter of from about 10 nm to about 100,000 nm, about 30 nm to about 1000 nm, about 30 nm to about 500 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 200 nm, 250 nm, 300 nm, 310 nm, 375 nm, 400 nm, 500 nm, 800 nm, 1000 nm, 1250 nm, 1400 nm, or 1500 nm. For example, in some embodiments, the the nanoparticle has a mean diameter of from about 10 nm to about 1,000 nm

In various embodiments, the nanoparticle is substantially non-toxic.

In various embodiments, the nanoparticle is biodegradable.

In one aspect of the invention, the nanoparticle comprises at least one cargo. In various aspects, the invention is not limited to any particular cargo or otherwise agent for which the nanoparticle is able to carry or transport. Rather, the invention includes any agent that can be carried by the nanoparticle. For example, agents that can be carried by the nanoparticle of the invention include, but are not limited to, diagnostic agents, detectable agents, and therapeutic agents. Thus, in various embodiments, the nanoparticle comprises at least one agent. In other embodiments, the nanoparticle encapsulates at least one agent.

In some embodiments, the weight ratio of the amphiphilic Janus dendrimer: the at least one agent is between about 1:1 to about 10,000:1. In some embodiments, the nanoparticle comprises, or encapsulates, at least one agent. In some embodiments, the weight ratio of the amphiphilic Janus dendrimer: the at least one agent is between about 2:1 to about 1,000:1. In some embodiments, the nanoparticle comprises, or encapsulates, at least one agent. In some embodiments, the weight 5 ratio of the amphiphilic Janus dendrimer: the at least one agent is between about 3:1 to about 10:1. In some embodiments, the nanoparticle comprises, or encapsulates, at least one agent. In some embodiments, the weight ratio of the amphiphilic Janus dendrimer: the at least one agent is between about 4:1 to about 10:1. In some embodiments, the nanoparticle comprises, or encapsulates, at least one agent. In some embodiments, the weight ratio of the amphiphilic Janus dendrimer: the at least one agent is between about 5:1 to about 10:1. In some embodiments, the nanoparticle comprises, or encapsulates, at least one agent. In some embodiments, the weight ratio of the amphiphilic Janus dendrimer: the at least one agent is between about 6:1 to about 10:1. In some embodiments, the nanoparticle comprises, or encapsulates, at least one agent. In some embodiments, the weight ratio of the amphiphilic Janus dendrimer: the at least one agent is between about 7:1 to about 10:1. In some embodiments, the nanoparticle comprises, or encapsulates, at least one agent. In some embodiments, the weight ratio of the amphiphilic Janus dendrimer: the at least one agent is between about 8:1 to about 10:1. In some embodiments, the nanoparticle comprises, or encapsulates, at least one agent. In some embodiments, the weight ratio of the amphiphilic Janus dendrimer: the at least one agent is between about 9:1 to about 10:1. In some embodiments, the nanoparticle comprises, or encapsulates, at least one agent. In some embodiments, the weight ratio of the amphiphilic Janus dendrimer: the at least one agent is between about 9.5:1 to about 10:1.

In various embodiments, the nanoparticle is suitable for delivering at least one cargo to a cell of interest.

For example, in some embodiments, the cargo is at least one agent comprising a diagnostic agent, detectable agent, therapeutic agent, nucleic acid molecule, gene editing agent, vaccine, composition for protein replacement therapy, or any combination thereof. In some embodiments, the at least one agent is selected from an mRNA, siRNA, microRNA, CRISPR-Cas9, sgRNA, small molecule, protein, antibody, peptide, protein, or any combination thereof. In some embodiments, the at least one agent comprises a nucleic acid molecule. In some embodiments, the nucleic acid molecule encodes at least one selected from an antigen, antibody, gene editing molecule, chimeric antigen receptor (CAR), or any combination thereof. In some embodiments, the nucleic acid molecule is a DNA molecule or an RNA molecule. In some embodiments, the nucleic acid molecule is selected from cDNA, cRNA, CirRNA, mRNA, miRNA, siRNA, sgRNA, modified RNA, tRNA, antagomir, antisense molecule, targeted nucleic acid, or any combination thereof. In some embodiments, the modified RNA is a nucleoside-modified RNA. In some embodiments, the nucleoside-modified RNA comprises pseudouridine. In some embodiments, the nucleoside-modified RNA comprises pseudouridine plus 5-methyl-cytosine. In some embodiments, the nucleoside-modified RNA comprises 5-methyl-uridine. In some embodiments, the nucleoside-modified RNA comprises 1-methyl-pseudouridine.

Thus, in one embodiment, the nanoparticles may be used for the delivery of nucleoside-modified RNA to a subject in need thereof. In certain embodiments, delivery of a nucleoside-modified RNA to a subject comprises mixing the nucleoside-modified RNA with at least one dendrimer of Formula (I) prior to the step of contacting. In another embodiment, a method of invention further comprises administering nucleoside-modified RNA together with at least one dendrimer of Formula (I).

In some embodiments, customizable targeting can be achieved based on the identity of the linking group A. Additionally, the identity of the linking group A affects the delivery of the nanoparticle cargo. For an mRNA cargo it was found that an ester linking group resulted in delivery to the liver and/or spleen while an amide group favored delivery to the lungs. This allows the nanoparticle to be tailored to facilitate delivery to the desired target organ. One example of which would be the delivery of anti-inflammatory drugs to the lungs.

In another embodiment, the transfection reagent forms a nanoparticle, which is a liposome. Liposomes, in another embodiment, increase intracellular stability, increase uptake efficiency and improve biological activity. In another embodiment, liposomes are hollow spherical vesicles composed of dendrimers arranged in a similar fashion as those lipids which make up the cell membrane. They have, in another embodiment, an internal aqueous space for entrapping water-soluble compounds and range in size from 0.05 to several microns in diameter. In another embodiment, nanoparticle liposomes can deliver RNA to cells in a biologically active form.

In various embodiments, the nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, the nucleoside-modified RNA, when present in the nanoparticles, is resistant in aqueous solution to degradation with a nuclease.

### Small Molecule Therapeutic Agents

In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the invention, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the invention, the therapeutic agent is synthesized via small library synthesis.

The small molecule and small molecule compounds described herein may be present as salts even if salts are not depicted, and it is understood that the invention embraces all salts and solvates of the therapeutic agents depicted here, as well as the non-salt and non-solvate form of the therapeutic agents, as is well understood by the skilled artisan. In some embodiments, the salts of the therapeutic agents of the invention are pharmaceutically acceptable salts.

Where tautomeric forms may be present for any of the therapeutic agents described herein, each and every tautomeric form is intended to be included in the invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the invention, such as crystalline or non-crystalline forms of the therapeutic agent. Compositions comprising a therapeutic agents of the invention are also intended, such as a composition of substantially pure therapeutic agent, including a specific stereochemical form thereof, or a composition comprising mixtures of therapeutic agents of the invention in any ratio, including two or more stereochemical forms, such as in a racemic or non-racemic mixture.

The invention also includes any or all active analog or derivative, such as a prodrug, of any therapeutic agent described herein. In one embodiment, the therapeutic agent is a prodrug. In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the invention can be used to treat a disease or disorder.

In one embodiment, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Therapeutic Agents

In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In one embodiment, the isolated nucleic acid molecule encodes a therapeutic peptide such a thrombomodulin, endothelial protein C receptor (EPCR), anti-thrombotic proteins including plasminogen activators and their mutants, antioxidant proteins including catalase, superoxide dismutase (SOD) and iron-sequestering proteins. In some embodiments, the therapeutic agent is an siRNA, miRNA, shRNA, or an antisense molecule, which inhibits a targeted nucleic acid including those encoding proteins that are involved in aggravation of the pathological processes.

In one embodiment, the nucleic acid comprises a promoter/regulatory sequence such that the nucleic acid is capable of directing expression of the nucleic acid. Thus, the invention encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In one embodiment, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the invention also includes methods of decreasing levels of PTPN22 using RNAi technology.

In one aspect, the invention includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a the delivery vehicle of the invention. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the invention to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrawal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl- methyl-, thio- and other modified forms of adenine, cytidine, guanine, thymine, and uridine.

In one embodiment of the invention, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In one embodiment, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In one embodiment, the therapeutic agent comprises a gRNA or a nucleic acid molecule encoding a gRNA. In one embodiment, the therapeutic agent comprises a Cas peptide or a nucleic acid molecule encoding a Cas peptide.

In one embodiment, the agent comprises a miRNA or a mimic of a miRNA. In one embodiment, the agent comprises a nucleic acid molecule that encodes a miRNA or mimic of a miRNA.

MiRNAs are small non-coding RNA molecules that are capable of causing post-transcriptional silencing of specific genes in cells by the inhibition of translation or through degradation of the targeted mRNA. A miRNA can be completely complementary or can have a region of noncomplementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, the miRNA includes a 2-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅Q. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,61 1, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In one embodiment, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In one embodiment, the oligonucleotide shares identity with endogenous miRNA or miRNA precursor nucleobase sequences. An oligonucleotide selected for inclusion in a composition of the invention may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the invention encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

### Nucleic Acids

In one embodiment, the invention includes a nanoparticle comprising or encapsulating one or more nucleic acid molecule. In one embodiment, the nucleic acid molecule is a nucleoside-modified mRNA molecule. In one embodiment, the nucleoside-modified mRNA molecule encodes an antigen. In one embodiment, the nucleoside-modified mRNA molecule encodes a plurality of antigens. In certain embodiments, the nucleoside-modified mRNA molecule encodes an antigen that induces an adaptive immune response against the antigen. In one embodiment, the invention includes a nucleoside-modified mRNA molecule encoding an adjuvant.

The nucleotide sequences encoding an antigen or adjuvant, as described herein, can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the invention includes nucleotide sequences that are substantially homologous to the nucleotide sequences recited herein and encode an antigen or adjuvant of interest.

As used herein, a nucleotide sequence is “substantially homologous” to any of the nucleotide sequences described herein when its nucleotide sequence has a degree of identity with respect to the nucleotide sequence of at least 60%, advantageously of at least 70%, preferably of at least 85%, and more preferably of at least 95%. A nucleotide sequence that is substantially homologous to a nucleotide sequence encoding an antigen can typically be isolated from a producer organism of the antigen based on the information contained in the nucleotide sequence by means of introducing conservative or non-conservative substitutions, for example. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art.

Further, the scope of the invention includes nucleotide sequences that encode amino acid sequences that are substantially homologous to the amino acid sequences recited herein and preserve the immunogenic function of the original amino acid sequence.

As used herein, an amino acid sequence is “substantially homologous” to any of the amino acid sequences described herein when its amino acid sequence has a degree of identity with respect to the amino acid sequence of at least 60%, advantageously of at least 70%, preferably of at least 85%, and more preferably of at least 95%. The identity between two amino acid sequences is preferably determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

In one embodiment, the invention relates to a construct, comprising a nucleotide sequence encoding an antigen. In one embodiment, the construct comprises a plurality of nucleotide sequences encoding a plurality of antigens. For example, in certain embodiments, the construct encodes 1 or more, 2 or more, 5 or more, 10 or more, 15 or more, or 20 or more antigens. In one embodiment, the invention relates to a construct, comprising a nucleotide sequence encoding an adjuvant. In one embodiment, the construct comprises a first nucleotide sequence encoding an antigen and a second nucleotide sequence encoding an adjuvant.

In one embodiment, the composition comprises a plurality of constructs, each construct encoding one or more antigens. In certain embodiments, the composition comprises 1 or more, 2 or more, 5 or more, 10 or more, 15 or more, or 20 or more constructs. In one embodiment, the composition comprises a first construct, comprising a nucleotide sequence encoding an antigen; and a second construct, comprising a nucleotide sequence encoding an adjuvant.

In another particular embodiment, the construct is operatively bound to a translational control element. The construct can incorporate an operatively bound regulatory sequence for the expression of the nucleotide sequence of the invention, thus forming an expression cassette.

### Vectors

The nucleic acid sequences encapsulated in the nanoparticle of the invention can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid molecule of interest can be produced synthetically.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, sequencing vectors and vectors optimized for in vitro transcription.

In one embodiment, the composition of the invention comprises in vitro transcribed (IVT) RNA encoding an antigen. In one embodiment, the composition of the invention comprises IVT RNA encoding a plurality of antigens. In one embodiment, the composition of the invention comprises IVT RNA encoding an adjuvant. In one embodiment, the composition of the invention comprises IVT RNA encoding one or more antigens and one or more adjuvants.

### Nucleoside-Modified RNA

In one embodiment, the nucleic acid molecule comprises a nucleoside-modified RNA. Nucleoside-modified mRNA have particular advantages over non-modified mRNA, including for example, increased stability, low or absent innate immunogenicity, and enhanced translation. Nucleoside-modified mRNA useful in the invention is further described in U.S. Pat. No. 8,278,036, which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days (Karikó et al., 2008, Mol Ther 16:1833-1840; Karikó et al., 2012, Mol Ther 20:948-953). The amount of mRNA required to exert a physiological effect is small and that makes it applicable for human therapy. For example, as described herein, nucleoside-modified mRNA encoding an antigen has demonstrated the ability to induce CD4+ and CD8+ T-cell and antigen-specific antibody production. For example, in certain instances, antigen encoded by nucleoside-modified mRNA induces greater production of antigen-specific antibody production as compared to antigen encoded by non-modified mRNA.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Karikó et al., 2011, Nucleic Acids Research 39:e142; Karikó et al., 2012, Mol Ther 20:948-953; Karikó et al., 2005, Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). A preparative HPLC purification procedure has been established that was critical to obtain pseudouridine-containing RNA that has superior translational potential and no innate immunogenicity (Karikó et al., 2011, Nucleic Acids Research 39:e142). Administering HPLC-purified, pseudourine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy.

The invention encompasses RNA, oligoribonucleotide, and polyribonucleotide molecules comprising pseudouridine or a modified nucleoside. In certain embodiments, the composition comprises an isolated nucleic acid encoding an antigen, wherein the nucleic acid comprises a pseudouridine or a modified nucleoside. In certain embodiments, the composition comprises a vector, comprising an isolated nucleic acid encoding an antigen, adjuvant, or combination thereof, wherein the nucleic acid comprises a pseudouridine or a modified nucleoside.

In one embodiment, the nucleoside-modified RNA of the invention is IVT RNA. For example, in certain embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In one embodiment, the modified nucleoside is m¹acp³ψ (1-methyl-3-(3-amino-3-carboxypropyl) pseudouridine. In another embodiment, the modified nucleoside is m¹ψ (1-methylpseudouridine). In another embodiment, the modified nucleoside is ψm (2′-O-methylpseudouridine. In another embodiment, the modified nucleoside is m⁵D (5-methyldihydrouridine). In another embodiment, the modified nucleoside is m³ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the nucleoside that is modified in the nucleoside-modified RNA the invention is uridine (U). In another embodiment, the modified nucleoside is cytidine (C). In another embodiment, the modified nucleoside is adenosine (A). In another embodiment the modified nucleoside is guanosine (G).

In another embodiment, the modified nucleoside of the invention is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is ψ (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m6A (2-methylthio-N⁶-methyladenosine); i⁶A (N⁶-isopentenyladenosine); ms²i⁶A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); miI (1-methylinosine); miIm (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²2G (N²,N²-dimethylguanosine); m²Gm (N²,2′-O-dimethylguanosine); m²2Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G (archaeosine); D (dihydrouridine); m⁵Um (5,2′-O-dimethyluridine); s⁴U (4-thiouridine); m⁵s2U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s2U (5-methoxycarbonylmethyl-2-thiouridine); nm⁵s2U (5-aminomethyl-2-thiouridine); mnm⁵U (5-methylaminomethyluridine); mnm⁵s2U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s2U (5-carboxymethylaminomethyl-2-thiouridine); m⁶2A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶2Am (N⁶,N⁶,O-2′-trimethyladenosine); m²° 7G (N²,7-dimethylguanosine); m²,2° ⁷G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s2U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In another embodiment, between 0.1% and 100% of the residues in the nucleoside-modified of the invention are modified (e.g. either by the presence of pseudouridine or a modified nucleoside base). In another embodiment, 0.1% of the residues are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of the given nucleotide that is modified is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, a nucleoside-modified RNA of the invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 5-fold factor. In another embodiment, translation is enhanced by a 7-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In another embodiment, the nucleoside-modified antigen-encoding RNA of the invention induces significantly more adaptive immune response than an unmodified in vitro-synthesized RNA molecule with the same sequence. In another embodiment, the modified RNA molecule exhibits an adaptive immune response that is 2-fold greater than its unmodified counterpart. In another embodiment, the adaptive immune response is increased by a 3-fold factor. In another embodiment the adaptive immune response is increased by a 5-fold factor. In another embodiment, the adaptive immune response is increased by a 7-fold factor. In another embodiment, the adaptive immune response is increased by a 10-fold factor. In another embodiment, the adaptive immune response is increased by a 15-fold factor. In another embodiment the adaptive immune response is increased by a 20-fold factor. In another embodiment, the adaptive immune response is increased by a 50-fold factor. In another embodiment, the adaptive immune response is increased by a 100-fold factor. In another embodiment, the adaptive immune response is increased by a 200-fold factor. In another embodiment, the adaptive immune response is increased by a 500-fold factor. In another embodiment, the adaptive immune response is increased by a 1000-fold factor. In another embodiment, the adaptive immune response is increased by a 2000-fold factor. In another embodiment, the adaptive immune response is increased by another fold difference.

In another embodiment, “induces significantly more adaptive immune response” refers to a detectable increase in an adaptive immune response. In another embodiment, the term refers to a fold increase in the adaptive immune response (e.g., 1 of the fold increases enumerated above). In another embodiment, the term refers to an increase such that the nucleoside-modified RNA can be administered at a lower dose or frequency than an unmodified RNA molecule with the same species while still inducing an effective adaptive immune response. In another embodiment, the increase is such that the nucleoside-modified RNA can be administered using a single dose to induce an effective adaptive immune response.

In another embodiment, the nucleoside-modified RNA of the invention exhibits significantly less innate immunogenicity than an unmodified in vitro-synthesized RNA molecule with the same sequence. In another embodiment, the modified RNA molecule exhibits an innate immune response that is 2-fold less than its unmodified counterpart. In another embodiment, innate immunogenicity is reduced by a 3-fold factor. In another embodiment, innate immunogenicity is reduced by a 5-fold factor. In another embodiment, innate immunogenicity is reduced by a 7-fold factor. In another embodiment, innate immunogenicity is reduced by a 10-fold factor. In another embodiment, innate immunogenicity is reduced by a 15-fold factor. In another embodiment, innate immunogenicity is reduced by a 20-fold factor. In another embodiment, innate immunogenicity is reduced by a 50-fold factor. In another embodiment, innate immunogenicity is reduced by a 100-fold factor. In another embodiment, innate immunogenicity is reduced by a 200-fold factor. In another embodiment, innate immunogenicity is reduced by a 500-fold factor. In another embodiment, innate immunogenicity is reduced by a 1000-fold factor. In another embodiment, innate immunogenicity is reduced by a 2000-fold factor. In another embodiment, innate immunogenicity is reduced by another fold difference.

In another embodiment, “exhibits significantly less innate immunogenicity” refers to a detectable decrease in innate immunogenicity. In another embodiment, the term refers to a fold decrease in innate immunogenicity (e.g., 1 of the fold decreases enumerated above). In another embodiment, the term refers to a decrease such that an effective amount of the nucleoside-modified RNA can be administered without triggering a detectable innate immune response. In another embodiment, the term refers to a decrease such that the nucleoside-modified RNA can be repeatedly administered without eliciting an innate immune response sufficient to detectably reduce production of the recombinant protein. In another embodiment, the decrease is such that the nucleoside-modified RNA can be repeatedly administered without eliciting an innate immune response sufficient to eliminate detectable production of the recombinant protein.

### Polypeptide Therapeutic Agents

In other related aspects, the therapeutic agent includes an isolated peptide that modulates a target. For example, in one embodiment, the peptide of the invention inhibits or activates a target directly by binding to the target thereby modulating the normal functional activity of the target. In one embodiment, the peptide of the invention modulates the target by competing with endogenous proteins. In one embodiment, the peptide of the invention modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

The nanoparticles may further comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids. In one embodiment, the nanoparticles do not further comprise any lipid capable of forming a particle to which the one or more nucleic acid molecules are attached, or in which the one or more nucleic acid molecules are encapsulated. In one embodiment, the nanoparticles do not further comprise any or all of a simple lipid, a compound lipid, or a derived lipid.

In one embodiment, the nanoparticle comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease. In one embodiment, the nanoparticle does not comprise a cationic lipid.

In certain embodiments, the cationic lipid which is optionally present or not present in the nanoparticles comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N (N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate 5 (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Such amino lipids include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In various embodiments, the nanoparticles further comprise a steroid or steroid analogue. A “steroid” is a compound comprising the following carbon skeleton:

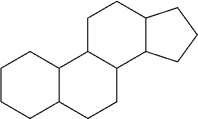 [SMILES: C1CCC2C(C1)CCC1C3CCCC3CCC21]

In certain embodiments, the steroid or steroid analogue is cholesterol. In one embodiment, the nanoparticles do not comprise a steroid or steroid analogue. In one embodiment, the nanoparticles do not comprise cholesterol.

In one embodiment, the nanoparticles further comprise a stabilizer. In one embodiment, the stabilizer comprises oligooxyetylenes. In one embodiment, the stabilizer comprises a water soluble macromolecule. In one embodiment the stabilizer comprises a water soluble oligomer. In one embodiment, the stabilizer comprises a carbohydrate.

In certain embodiments, the nanoparticle comprises one or more targeting moieties which are capable of targeting the nanoparticle to a cell or cell population. For example, in one embodiment, the targeting moiety is a ligand which directs the nanoparticle to a receptor found on a cell surface.

In certain embodiments, the nanoparticle comprises one or more internalization domains. For example, in one embodiment, the nanoparticle comprises one or more domains which bind to a cell to induce the internalization of the nanoparticle. For example, in one embodiment, the one or more internalization domains bind to a receptor found on a cell surface to induce receptor-mediated uptake of the nanoparticle. In certain embodiments, the nanoparticle is capable of binding a biomolecule in vivo, where the nanoparticle-bound biomolecule can then be recognized by a cell-surface receptor to induce internalization. For example, in one embodiment, the nanoparticle binds systemic ApoE, which leads to the uptake of the nanoparticle and associated cargo.

### Compositions

In one aspect, the present invention relates to compositions comprising at least one amphiphilic Janus dendrimer of the presenting invention and/or nanoparticle thereof. In some embodiments, the composition further comprises at least one agent described herein.

The invention also relates to compositions comprising at least one compound of Formula (I) and methods of use thereof for delivering an encapsulated agent to a site of interest. Exemplary agents that can be encapsulated in the compositions of the invention include, but are not limited to, diagnostic agents, detectable agents, and therapeutic agents.

In one embodiment, the composition comprises nanoparticles comprising a compound of Formula (I) and at least one agent encapsulated by the nanoparticle. In some embodiments, the encapsulated agent comprises an agent for inducing an immune response in a subject. In certain embodiments, the invention provides a composition comprising a nanoparticle encapsulating a nucleic acid molecule encoding an agent for inducing an immune response in a subject. For example, in certain embodiments, the composition comprises a vaccine comprising a nucleic acid molecule encoding an antigen.

In one embodiment, the composition is a vaccine.

In one embodiment, the composition comprises a nanoparticle and one or more nucleic acid molecules described herein. For example, in one embodiment, the composition comprises a nanoparticle and one or more nucleoside-modified RNA molecules encoding one or more antigens, adjuvants, or a combination thereof.

In one embodiment, the composition may be prepared by injection of a mixture comprising a compound described herein into a suitable solution, such as a solution comprising the agent to be encapsulated. In one embodiment, microfluidic techniques such as those required for the formation of lipid nanoparticles (LNPs) are not required for the production of the inventive nanoparticles.

In one embodiment, the composition of the invention comprises in vitro transcribed (IVT) RNA molecule. For example, in certain embodiments, the composition of the invention comprises IVT RNA molecule which encodes an agent. In certain embodiments, the IVT RNA molecule of the present composition is a nucleoside-modified mRNA molecule. In certain embodiments, the agent is at least one of a viral antigen, bacterial antigen, fungal antigen, parasitic antigen, tumor-specific antigen, or tumor-associated antigen. However, the invention is not limited to any particular agent or combination of agents. In certain embodiments, the composition comprises an adjuvant. In certain embodiments, the composition comprises a nucleic acid molecule encoding an adjuvant. In one embodiment, the composition comprises a nucleoside-modified RNA encoding an adjuvant.

In one embodiment, the composition comprises at least one nucleoside-modified RNA molecule encoding a combination of at least two agents. In one embodiment, the composition comprises a combination of two or more nucleoside-modified RNA molecules encoding a combination of two or more agents.

In one embodiment, the invention provides a method for inducing an immune response in a subject. For example, the method can be used to provide immunity in the subject against a virus, bacteria, fungus, parasite, cancer, or the like. In some embodiments, the method comprises administering to the subject a composition comprising one or more nanoparticles comprising one or more nucleoside-modified RNA encoding at least one antigen, an adjuvant, or a combination thereof.

In one embodiment, the method comprises the systemic administration of the composition into the subject, including for example intradermal administration. In certain embodiments, the method comprises administering a plurality of doses to the subject. In another embodiment, the method comprises administering a single dose of the composition, where the single dose is effective in inducing a therapeutic response.

### Vaccine

In one embodiment, the invention provides an immunogenic composition for inducing an immune response in a subject. For example, in one embodiment, the immunogenic composition is a vaccine. As used herein, an “immunogenic composition” may comprise an antigen (e.g., a peptide or polypeptide), a nucleic acid encoding an antigen, a cell expressing or presenting an antigen or cellular component, or a combination thereof. In particular embodiments the composition comprises or encodes all or part of any peptide antigen, or an immunogenically functional equivalent thereof. In other embodiments, the composition comprises a mixture of mRNA molecules that encodes one or more additional immunostimulatory agent. Immunostimulatory agents include, but are not limited to, an additional antigen, an immunomodulator, or an adjuvant. In the context of the invention, the term “vaccine” refers to a substance that induces immunity upon inoculation into animals.

A vaccine of the invention may vary in its composition of nucleic acid components. In a non-limiting example, a nucleic acid encoding an antigen might also be formulated with an adjuvant. Of course, it will be understood that various compositions described herein may further comprise additional components. A vaccine of the invention, and its various components, may be prepared and/or administered by any method disclosed herein or as would be known to one of ordinary skill in the art, in light of the present disclosure.

The induction of the immunity by the expression of the antigen can be detected by observing in vivo or in vitro the response of all or any part of the immune system in the host against the antigen.

For example, a method for detecting the induction of cytotoxic T lymphocytes is well known. A foreign substance that enters the living body is presented to T cells and B cells by the action of APCs. T cells that respond to the antigen presented by APC in an antigen specific manner differentiate into cytotoxic T cells (also referred to as cytotoxic T lymphocytes or CTLs) due to stimulation by the antigen. These antigen stimulated cells then proliferate. This process is referred to herein as “activation” of T cells. Therefore, CTL induction by an epitope of a polypeptide or peptide or combinations thereof can be evaluated by presenting an epitope of a polypeptide or peptide or combinations thereof to a T cell by APC, and detecting the induction of CTL. Furthermore, APCs have the effect of activating B cells, CD4+ T cells, CD8+ T cells, macrophages, eosinophils and NK cells.

A method for evaluating the inducing action of CTL using dendritic cells (DCs) as APC is well known in the art. DC is a representative APC having a robust CTL inducing action among APCs. In the methods of the invention, the epitope of a polypeptide or peptide or combinations thereof is initially expressed by the DC and then this DC is contacted with T cells. Detection of T cells having cytotoxic effects against the cells of interest after the contact with DC shows that the an epitope of a polypeptide or peptide or combinations thereof has an activity of inducing the cytotoxic T cells. Furthermore, the induced immune response can be also examined by measuring IFN-gamma produced and released by CTL in the presence of antigen-presenting cells that carry immobilized peptide or combination of peptides by visualizing using anti-IFN-gamma antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7.

The antigens confirmed to possess CTL-inducing activity by these methods are antigens having DC activation effect and subsequent CTL-inducing activity. Furthermore, CTLs that have acquired cytotoxicity due to presentation of the antigen by APC can be also used as vaccines against antigen-associated disorders.

The induction of immunity by expression of the antigen can be further confirmed by observing the induction of antibody production against the antigen. For example, when antibodies against an antigen are induced in a laboratory animal immunized with the composition encoding the antigen, and when antigen-associated pathology is suppressed by those antibodies, the composition is determined to induce immunity.

The induction of immunity by expression of the antigen can be further confirmed by observing the induction of CD4+ T cells. CD4+ T cells can also lyse target cells, but mainly supply help in the induction of other types of immune responses, including CTL and antibody generation. The type of CD4+ T cell help can be characterized, as Th1, Th2, Th9, Th17, Tregulatory, or T follicular helper (T_{fh}) cells. Each subtype of CD4+ T cell supplies help to certain types of immune responses. Of particular interest to this invention, the T_{fh} subtype provides help in the generation of high affinity antibodies.

In some embodiments, the therapeutic compounds or compositions of the invention may be administered prophylactically (i.e., to prevent disease or disorder) or therapeutically (i.e., to treat disease or disorder) to subjects suffering from or at risk of (or susceptible to) developing the disease or disorder. Such subjects may be identified using standard clinical methods. In the context of the invention, prophylactic administration occurs prior to the manifestation of overt clinical symptoms of disease, such that a disease or disorder is prevented or alternatively delayed in its progression. In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

### Targeting Domain

In one embodiment, the composition comprises a targeting domain that directs the delivery vehicle to a site. In one embodiment, the site is a site in need of the agent comprised within the delivery vehicle. The targeting domain may comprise a nucleic acid, peptide, antibody, small molecule, organic molecule, inorganic molecule, glycan, sugar, hormone, and the like that targets the particle to a site in particular need of the therapeutic agent. In certain embodiments, the particle comprises multivalent targeting, wherein the particle comprises multiple targeting mechanisms described herein. In certain embodiments, the targeting domain of the delivery vehicle specifically binds to a target associated with a site in need of an agent comprised within the delivery vehicle. For example, the targeting domain may be chosen to recognize a ligand that acts as a cell surface marker on target cells associated with a particular disease state. Such a target can be a protein, protein fragment, antigen, or other biomolecule that is associated with the targeted site. In some embodiments, the targeting domain is an affinity ligand which specifically binds to a target. In certain embodiments, the target (e.g. antigen) associated with a site in need of a treatment with an agent. In some embodiments, the targeting domain may be co-polymerized with the composition comprising the delivery vehicle. In some embodiments, the targeting domain may be covalently attached to the composition comprising the delivery vehicle, such as through a chemical reaction between the targeting domain and the composition comprising the delivery vehicle. In some embodiments, the targeting domain is an additive in the delivery vehicle. Targeting domains of the instant invention include, but are not limited to, antibodies, antibody fragments, proteins, peptides, and nucleic acids.

In various embodiments, the targeting domain binds to a cell surface molecule of a cell of interest. For example, in various embodiments, the targeting domain binds to a cell surface molecule of an endothelial cell, a stem cell, or an immune cell.

### Peptides

In one embodiment, the targeting domain of the invention comprises a peptide. In certain embodiments, the peptide targeting domain specifically binds to a target of interest.

The peptide of the invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the invention can be post-translationally modified. For example, post-translational modifications that fall within the scope of the invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation.

### Nucleic Acids

In one embodiment, the targeting domain of the invention comprises an isolated nucleic acid, including for example a DNA oligonucleotide and a RNA oligonucleotide. In certain embodiments, the nucleic acid targeting domain specifically binds to a target of interest. For example, in one embodiment, the nucleic acid comprises a nucleotide sequence that specifically binds to a target of interest.

The nucleotide sequences of a nucleic acid targeting domain can alternatively comprise sequence variations with respect to the original nucleotide sequences, for example, substitutions, insertions and/or deletions of one or more nucleotides, with the condition that the resulting nucleic acid functions as the original and specifically binds to the target of interest.

In the sense used in this description, a nucleotide sequence is “substantially homologous” to any of the nucleotide sequences describe herein when its nucleotide sequence has a degree of identity with respect to the nucleotide sequence of at least 60%, advantageously of at least 70%, preferably of at least 85%, and more preferably of at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm [BLAST Manual, Altschul, S., et al., NCBI NLM NIH Bethesda, Md. 20894, Altschul, S., et al., J. Mol. Biol. 215: 403-410 (1990)].

### Antibodies

In one embodiment, the targeting domain of the invention comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Adjuvant

In one embodiment, the composition comprises an adjuvant. In one embodiment, the composition comprises a nucleic acid molecule encoding an adjuvant. In one embodiment, the adjuvant-encoding nucleic acid molecule is IVT RNA. In one embodiment, the adjuvant-encoding nucleic acid molecule is nucleoside-modified mRNA.

Exemplary adjuvants include, but is not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MIHC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R², TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, INK, interferon response genes, NFkB, Bax, TRAIL, TRAILrec, TRAILrecDRC5, TRAIL-R³, TRAIL-R⁴, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

### Antibody Therapeutic Agents

The invention also contemplates a delivery vehicle comprising an antibody, or antibody fragment, specific for a target. That is, the antibody can inhibit a target to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain FV molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

### Antigen

The invention provides a composition that induces a therapeutic response in a subject. In one embodiment, the composition comprises an antigen. In one embodiment, the composition comprises a nucleic acid sequence which encodes an antigen. For example, in certain embodiments, the composition comprises a nucleoside-modified RNA encoding an antigen. The antigen may be any molecule or compound, including but not limited to a polypeptide, peptide or protein that induces a therapeutic response, such as an adaptive immune response, in a subject.

In one embodiment, the antigen comprises a polypeptide or peptide associated with a pathogen, such that the antigen induces an adaptive immune response against the antigen, and therefore the pathogen. In one embodiment, the antigen comprises a fragment of a polypeptide or peptide associated with a pathogen, such that the antigen induces an adaptive immune response against the pathogen.

In certain embodiments, the antigen comprises an amino acid sequence that is substantially homologous to the amino acid sequence of an antigen described herein and retains the immunogenic function of the original amino acid sequence. For example, in certain embodiments, the amino acid sequence of the antigen has a degree of identity with respect to the original amino acid sequence of at least 60%, advantageously of at least 70%, preferably of at least 85%, and more preferably of at least 95%.

In one embodiment, the antigen is encoded by a nucleic acid sequence of a nucleic acid molecule. In certain embodiments, the nucleic acid sequence comprises DNA, RNA, cDNA, a variant thereof, a fragment thereof, or a combination thereof. In one embodiment, the nucleic acid sequence comprises a modified nucleic acid sequence. For example, in one embodiment the antigen-encoding nucleic acid sequence comprises nucleoside-modified RNA, as described in detail elsewhere herein. In certain instances, the nucleic acid sequence comprises include additional sequences that encode linker or tag sequences that are linked to the antigen by a peptide bond.

In certain embodiments, the antigen, encoded by the nucleoside-modified nucleic acid molecule, comprises a protein, peptide, a fragment thereof, or a variant thereof, or a combination thereof from any number of organisms, for example, a virus, a parasite, a bacterium, a fungus, or a mammal. For example, in certain embodiments, the antigen is associated with an autoimmune disease, allergy, or asthma. In other embodiments, the antigen is associated with cancer, herpes, influenza, hepatitis B, hepatitis C, human papilloma virus (HPV), ebola, pneumococcus, Haemophilus influenza, meningococcus, dengue, tuberculosis, malaria, norovirus or human immunodeficiency virus (HIV). In certain embodiments, the antigen comprises a consensus sequence based on the amino acid sequence of two or more different organisms. In certain embodiments, the nucleic acid sequence encoding the antigen is optimized for effective translation in the organism in which the composition is delivered.

In one embodiment, the antigen comprises a tumor-specific antigen or tumor-associated antigen, such that the antigen induces an adaptive immune response against the tumor. In one embodiment, the antigen comprises a fragment of a tumor-specific antigen or tumor-associated antigen, such that the antigen induces an adaptive immune response against the tumor. In certain embodiment, the tumor-specific antigen or tumor-associated antigen is a mutation variant of a host protein.

### Viral Antigens

In one embodiment, the antigen comprises a viral antigen, or fragment thereof, or variant thereof. In certain embodiments, the viral antigen is from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. In certain embodiments, the viral antigen is from papilloma viruses, for example, human papillomoa virus (HPV), human immunodeficiency virus (HIV), polio virus, hepatitis B virus, hepatitis C virus, smallpox virus (Variola major and minor), vaccinia virus, influenza virus, rhinoviruses, dengue fever virus, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, chikungunya virus, lassa virus, arenavirus, severe acute respiratory syndrome (SARS) virus, severe acute respiratory syndrome coronavirus 2 (SARS-CoV-2) or a cancer causing virus.

### Parasite Antigens

In certain embodiments, the antigen comprises a parasite antigen or fragment or variant thereof. In certain embodiments, the parasite is a protozoa, helminth, or ectoparasite. In certain embodiments, the helminth (i.e., worm) is a flatworm (e.g., flukes and tapeworms), a thorny-headed worm, or a round worm (e.g., pinworms). In certain embodiments, the ectoparasite is lice, fleas, ticks, and mites.

In certain embodiments, the parasite is any parasite causing the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

In certain embodiments, the parasite is Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus—lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

### Bacterial Antigens

In one embodiment, the antigen comprises a bacterial antigen or fragment or variant thereof. In certain embodiments, the bacterium is from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

In certain embodiments, the bacterium is a gram positive bacterium or a gram negative bacterium. In certain embodiments, the bacterium is an aerobic bacterium or an anaerobic bacterium. In certain embodiments, the bacterium is an autotrophic bacterium or a heterotrophic bacterium. In certain embodiments, the bacterium is a mesophile, a neutrophile, an extremophile, an acidophil, an alkaliphile, a thermophile, psychrophile, halophile, or an osmophile.

In certain embodiments, the bacterium is an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. In certain embodiments, bacterium is a mycobacteria, Clostridium tetani, Yersinia pestis, Bacillus anthracis, methicillin-resistant Staphylococcus aureus (MRSA), or Clostridium difficile.

### Fungal Antigens

In one embodiment, the antigen comprises a fungal antigen or fragment or variant thereof. In certain embodiments, the fungus is Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

### Tumor Antigens

In certain embodiments, the antigen comprises a tumor antigen, including for example a tumor-associated antigen or a tumor-specific antigen. In the context of the invention, “tumor antigen” or “hyperporoliferative disorder antigen” or “antigen associated with a hyperproliferative disorder” refer to antigens that are common to specific hyperproliferative disorders. In certain aspects, the hyperproliferative disorder antigens of the invention are derived from cancers including, but not limited to, primary or metastatic melanoma, mesothelioma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

Tumor antigens are proteins that are produced by tumor cells that elicit an immune response, particularly T-cell mediated immune responses. In one embodiment, the tumor antigen of the invention comprises one or more antigenic cancer epitopes immunogenically recognized by tumor infiltrating lymphocytes (TIL) derived from a cancer tumor of a mammal. The selection of the antigen will depend on the particular type of cancer to be treated or prevented by way of the composition of the invention.

Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H₄-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H₁, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90Mac-2 binding protein\cyclophilin C-associated protein, TAAL6, TAG72, TLP, and TPS.

In a preferred embodiment, the antigen includes but is not limited to CD19, CD20, CD22, ROR1, Mesothelin, CD33/IL3Ra, c-Met, PSMA, Glycolipid F77, EGFRvIII, GD-2, MY-ESO-1 TCR, MAGE A3 TCR, and the like.

### Combinations

In one embodiment, the composition of the invention comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, in one embodiment, the composition comprises a 1:1 ratio of two individual agents. However, the combination is not limited to any particular ratio. Rather any ratio that is shown to be effective is encompassed.

### Conjugation

In various embodiments of the invention, the delivery vehicle is conjugated to a targeting domain. Exemplary methods of conjugation can include, but are not limited to, covalent bonds, electrostatic interactions, and hydrophobic (“van der Waals”) interactions. In one embodiment, the conjugation is a reversible conjugation, such that the delivery vehicle can be disassociated from the targeting domain upon exposure to certain conditions or chemical agents. In another embodiment, the conjugation is an irreversible conjugation, such that under normal conditions the delivery vehicle does not dissociate from the targeting domain.

In some embodiments, the conjugation comprises a covalent bond between an activated polymer conjugated lipid and the targeting domain. The term “activated polymer conjugated lipid” refers to a molecule comprising a lipid portion and a polymer portion that has been activated via functionalization of a polymer conjugated lipid with a first coupling group. In one embodiment, the activated polymer conjugated lipid comprises a first coupling group capable of reacting with a second coupling group. In one embodiment, the activated polymer conjugated lipid is an activated pegylated lipid. In one embodiment, the first coupling group is bound to the lipid portion of the pegylated lipid. In another embodiment, the first coupling group is bound to the polyethylene glycol portion of the pegylated lipid. In one embodiment, the second functional group is covalently attached to the targeting domain.

The first coupling group and second coupling group can be any functional groups known to those of skill in the art to together form a covalent bond, for example under mild reaction conditions or physiological conditions. In some embodiments, the first coupling group or second coupling group are selected from the group consisting of maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctyne, aldehydes, and sulfhydryl groups. In some embodiments, the first coupling group or second coupling group is selected from the group consisiting of free amines (—NH₂), free sulfhydryl groups (—SH), free hydroxide groups (—OH), carboxylates, hydrazides, and alkoxyamines. In some embodiments, the first coupling group is a functional group that is reactive toward sulfhydryl groups, such as maleimide, pyridyl disulfide, or a haloacetyl. In one embodiment, the first coupling group is a maleimide.

In one embodiment, the second coupling group is a sulfhydryl group. The sulfhydryl group can be installed on the targeting domain using any method known to those of skill in the art. In one embodiment, the sulfhydryl group is present on a free cysteine residue. In one embodiment, the sulfhydryl group is revealed via reduction of a disulfide on the targeting domain, such as through reaction with 2-mercaptoethylamine. In one embodiment, the sulfhydryl group is installed via a chemical reaction, such as the reaction between a free amine and 2-iminothilane or N-succinimidyl S-acetylthioacetate (SATA).

In some embodiments, the polymer conjugated lipid and targeting domain are functionalized with groups used in “click” chemistry. Bioorthogonal “click” chemistry comprises the reaction between a functional group with a 1,3-dipole, such as an azide, a nitrile oxide, a nitrone, an isocyanide, and the link, with an alkene or an alkyne dipolarophiles. Exemplary dipolarophiles include any strained cycloalkenes and cycloalkynes known to those of skill in the art, including, but not limited to, cyclooctynes, dibenzocyclooctynes, monofluorinated cyclcooctynes, difluorinated cyclooctynes, and biarylazacyclooctynone

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### Treatment Methods

The invention provides methods of delivering an agent to a cell, tissue, or organ of a subject. In some embodiments, the agent is a diagnostic agent to detect at least one marker associated with a disease or disorder. In some embodiments, the agent is a therapeutic agent for the treatment or prevention of a disease or disorder. Therefore, in some embodiments, the invention provides methods for diagnosing, treating or preventing a disease or disorder comprising administering an effective amount of a composition comprising one or more diagnostic or therapeutic agents, one or more adjuvants, or a combination thereof.

In some embodiments, the method provides immunity in the subject to an infection, disease, or disorder associated with an antigen. The invention thus provides a method of treating or preventing the infection, disease, or disorder associated with the antigen. For example, the method may be used to treat or prevent a viral infection, bacterial infection, fungal infection, parasitic infection, or cancer, depending upon the type of antigen of the administered composition. Exemplary antigens and associated infections, diseases, and tumors are described elsewhere herein.

In one embodiment, the composition is administered to a subject having an infection, disease, or cancer associated with the antigen. In one embodiment, the composition is administered to a subject at risk for developing the infection, disease, or cancer associated with the antigen. For example, the composition may be administered to a subject who is at risk for being in contact with a virus, bacteria, fungus, parasite, or the like. In one embodiment, the composition is administered to a subject who has increased likelihood, though genetic factors, environmental factors, or the like, of developing cancer.

In one embodiment, the method comprises administering a composition comprising one or more nucleoside-modified nucleic acid molecules encoding one or more antigens and one or more adjuvant. In one embodiment, the method comprises administering a composition comprising a first nucleoside-modified nucleic acid molecule encoding one or more antigens and a second nucleoside-modified nucleic acid molecule encoding one or more adjuvants. In one embodiment, the method comprises administering a first composition comprising one or more nucleoside-modified nucleic acid molecules encoding one or more antigens and administering a second composition comprising one or more nucleoside-modified nucleic acid molecules encoding one or more adjuvants.

In certain embodiments, the method comprises administering to subject a plurality of nucleoside-modified nucleic acid molecules encoding a plurality of antigens, adjuvants, or a combination thereof.

In certain embodiments, the method of the invention allows for sustained expression of the antigen or adjuvant, described herein, for at least several days following administration. However, the method, in certain embodiments, also provides for transient expression, as in certain embodiments, the nucleic acid is not integrated into the subject genome.

In certain embodiments, the method comprises administering nucleoside-modified RNA which provides stable expression of the antigen or adjuvant described herein. In some embodiments, administration of nucleoside-modified RNA results in little to no innate immune response, while inducing an effective adaptive immune response.

Administration of the compositions of the invention in a method of treatment can be achieved in a number of different ways, using methods known in the art. In one embodiment, the method of the invention comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition.

In one embodiment, the method comprises inhalation of the composition. In one embodiment, the method comprises intranasal delivery of the composition.

It will be appreciated that the composition of the invention may be administered to a subject either alone, or in conjunction with another agent.

The therapeutic and prophylactic methods of the invention thus encompass the use of pharmaceutical compositions encoding an antigen, adjuvant, or a combination thereof, described herein to practice the methods of the invention. The pharmaceutical compositions useful for practicing the invention may be administered to deliver a dose of from ng/kg/day and 100 mg/kg/day. In one embodiment, the invention envisions administration of a dose which results in a concentration of the compound of the invention from 10 nM and 10 μM in a mammal.

Typically, dosages which may be administered in a method of the invention to a mammal, preferably a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 0.1 μg to about 10 mg per kilogram of body weight of the mammal. More preferably, the dosage will vary from about 1 pg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of an immunogenic composition or vaccine of the invention may be performed by single administration or boosted by multiple administrations.

In one embodiment, the invention includes a method comprising administering one or more compositions encoding one or more antigens or adjuvants described herein. In certain embodiments, the method has an additive effect, wherein the overall effect of the administering the combination is approximately equal to the sum of the effects of administering each antigen or adjuvant. In other embodiments, the method has a synergistic effect, wherein the overall effect of administering the combination is greater than the sum of the effects of administering each antigen or adjuvant.

### Treatment Methods for SARS-CoV-2

In one embodiment, the invention provides methods of inducing an adaptive immune response against SARS-CoV-2 in a subject comprising administering an effective amount of a composition comprising an amphiphilic Janus dendrimer of Formula (I) and one or more isolated nucleic acids encoding one or more SARS-CoV-2 antigens.

In one embodiment, the method provides immunity in the subject to SARS-CoV-2, SARS-CoV-2 infection, or to a disease or disorder associated with SARS-CoV-2. The invention thus provides a method of treating or preventing the infection, disease, or disorder associated with SARS-CoV-2. In one embodiment, the disease or disorder associated with SARS-CoV-2 is COVID-19 or a comorbidity of COVID-19.

In some embodiments, the invention is a method of administering to a subject a composition comprising at least one nucleoside-modified RNA encoding at least one SARS-CoV-2 antigen.

In one embodiment, the composition is administered to a subject having an infection, disease, or disorder associated with SARS-CoV-2. In one embodiment, the composition is administered to a subject at risk for developing the infection, disease, or disorder associated with SARS-CoV-2. For example, the composition may be administered to a subject who is at risk for being in contact with a SARS-CoV-2. In one embodiment, the composition is administered to a subject who lives in, traveled to, or is expected to travel to a geographic region in which SARS-CoV-2 is prevalent. In one embodiment, the composition is administered to a subject who is in contact with or expected to be in contact with another person who lives in, traveled to, or is expected to travel to a geographic region in which SARS-CoV-2 is prevalent. In one embodiment, the composition is administered to a subject who has knowingly been exposed to SARS-CoV-2 through their occupation or contact.

In one embodiment, the method comprises administering a composition comprising at least one dendrimer of Formula (I) and one or more nucleoside-modified nucleic acid molecules encoding one or more SARS-CoV-2 antigens and one or more adjuvant. In one embodiment, the method comprises administering a composition comprising a first nucleoside-modified nucleic acid molecule encoding one or more SARS-CoV-2 antigens and a second nucleoside-modified nucleic acid molecule encoding one or more adjuvants. In one embodiment, the method comprises administering a first composition comprising one or more nucleoside-modified nucleic acid molecules encoding one or more SARS-CoV-2 antigens and administering a second composition comprising one or more nucleoside-modified nucleic acid molecules encoding one or more adjuvants.

In certain embodiments, the method comprises administering to subject a plurality of nucleoside-modified nucleic acid molecules encoding a plurality of SARS-CoV-2 antigens, adjuvants, or a combination thereof.

In certain embodiments, the method of the invention allows for sustained expression of the SARS-CoV-2 antigen or adjuvant, described herein, for at least several days following administration. In certain embodiments, the method of the invention allows for sustained expression of the SARS-CoV-2 antigen or adjuvant, described herein, for at least 2 weeks following administration. In certain embodiments, the method of the invention allows for sustained expression of the SARS-CoV-2 antigen or adjuvant, described herein, for at least 1 month following administration. However, the method, in certain embodiments, also provides for transient expression, as in certain embodiments, the nucleic acid is not integrated into the subject genome.

In certain embodiments, the method comprises administering nucleoside-modified RNA, which provides stable expression of the SARS-CoV-2 antigen or adjuvant described herein. In some embodiments, administration of nucleoside-modified RNA results in little to no innate immune response, while inducing an effective adaptive immune response.

In certain embodiments, the method provides sustained protection against SARS-CoV-2. For example, in certain embodiments, the method provides sustained protection against SARS-CoV-2 for more than 2 weeks. In certain embodiments, the method provides sustained protection against SARS-CoV-2 for 1 month or more. In certain embodiments, the method provides sustained protection against SARS-CoV-2 for 2 months or more. In certain embodiments, the method provides sustained protection against SARS-CoV-2 for 3 months or more. In certain embodiments, the method provides sustained protection against SARS-CoV-2 for 4 months or more. In certain embodiments, the method provides sustained protection against SARS-CoV-2 for 5 months or more. In certain embodiments, the method provides sustained protection against SARS-CoV-2 for 6 months or more. In certain embodiments, the method provides sustained protection against SARS-CoV-2 for 1 year or more.

In one embodiment, a single immunization of the composition induces a sustained protection against SARS-CoV-2 for 1 month or more, 2 months or more, 3 months or more, 4 months or more, 5 months or more, 6 months or more, or 1 year or more.

Administration of the compositions of the invention in a method of treatment can be achieved in a number of different ways, using methods known in the art. In one embodiment, the method of the invention comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

It will be appreciated that the composition of the invention may be administered to a subject either alone, or in conjunction with another agent.

The therapeutic and prophylactic methods of the invention thus encompass the use of pharmaceutical compositions encoding a SARS-CoV-2 antigen, adjuvant, or a combination thereof, described herein to practice the methods of the invention. The pharmaceutical compositions useful for practicing the invention may be administered to deliver a dose of from 1 ng/kg/day and 100 mg/kg/day. In one embodiment, the invention envisions administration of a dose, which results in a concentration of the compound of the invention from 10 nM and 10 gM in a mammal.

Typically, dosages which may be administered in a method of the invention to a mammal, such as a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. In certain embodiments, the dosage of the compound will vary from about 0.1 pg to about 10 mg per kilogram of body weight of the mammal. In certain embodiments, the dosage will vary from about 1 pg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of an immunogenic composition or vaccine of the invention may be performed by single administration or boosted by multiple administrations.

In one embodiment, the invention includes a method comprising administering one or more compositions encoding one or more SARS-CoV-2 antigens or adjuvants described herein. In certain embodiments, the method has an additive effect, wherein the overall effect of the administering the combination is approximately equal to the sum of the effects of administering each SARS-CoV-2 antigen or adjuvant. In other embodiments, the method has a synergistic effect, wherein the overall effect of administering the combination is greater than the sum of the effects of administering each SARS-CoV-2 antigen or adjuvant.

### Experimental Examples

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the invention and practice the claimed methods. The following working examples therefore are not to be construed as limiting in any way the remainder of the disclosure.

### Example 1: One-Component Multifunctional Sequence-Defined Ionizable Amphiphilic Janus Dendrimer (IAJD) Delivery Systems of mRNA for Vaccines and Drugs

The present invention relates in part to a one-component multifunctional sequence-defined ionizable amphiphilic Janus dendrimer (IAJD) delivery system that co-assembles with mRNA by simple injection into dendrimersome nanoparticles (DNPs) (FIG. 1). Screening experiments with six libraries containing 52 IAJDs was performed both in-vitro and in vivo. They demonstrated the proof of concept of DNPs, their potential applications and utility as a model to elucidate fundamental aspects of nonviral vectors.

One of the major advantages of the synthetic vectors used for the delivery of mRNA consist in their unlimited synthetic capabilities. The transformation of the four-component LNP (FIG. 2) into a one-component DNP represents a demonstration of this synthetic capability.

FIG. 2 illustrates the process involved in the assembly of four-component LNPs. The four-components composition containing various ratios of the ionizable lipid, phospholipid, PEG-lipid and cholesterol is prepared as a solution in ethanol. This ethanol solution is mixed with a microfluidic device with a pH 3 to 5 buffer solution and with an aqueous solution of mRNA. The mRNA used in the acidic buffer solution is produced and stored in neutral water. The resulting nanoparticles are analyzed by dynamic light scattering (DLS) to determine the diameter (D, nm) and the polydispersity (PDI) of the LNPs, dialyzed to pH 7.4, analyzed by DLS again and stored at −70° C. before the in vitro or in vivo experiments are performed. FIG. 1 illustrates the same process for the one-component DNPs. The IAJD containing an ionizable amine incorporated in a precise sequence is dissolved in ethanol. The ethanol solution is injected into an acidic buffer solution containing mRNA (pH 3 to 5.2). Depending on the original pH of the buffer the resulting DNPs containing mRNA have already a pH between 4.5 and 7.3 and after DLS analysis can be used for in vitro and in vivo experiment without dialysis. Long time storage of DNPs is at 5° C. FIG. 1 also illustrates the transition from the extracellular to the intracellular process for both LNPs and DNPs. Once injected both LNP and DNP approach the corresponding cells and get encapsulated via endocytosis. The extracellular pH is 7.4 and therefore LNPs and DNPs enter the cell with an almost neutral surface. Then the endocytosis of LNPs or DNPs deposits them into endosomes, the pH of which decreases from 6.8 to 4.5 during their morphing into lysosomes due to the ATP-dependent proton pumps on the endosomal membrane (Huotari and Helenius, EMBO J. 2011, 30, 3481-3500). Therefore, both LNPs and DNPs get re-protonated, interact with the naturally occurring anionic lipids and release the mRNA that helps the ribosome to generate new proteins. When the ionizable amines from LNPs are segregated in their center, re-protonation of their periphery cannot occur and therefore, the release of mRNA in the cell has a very low efficiency (1-2%).

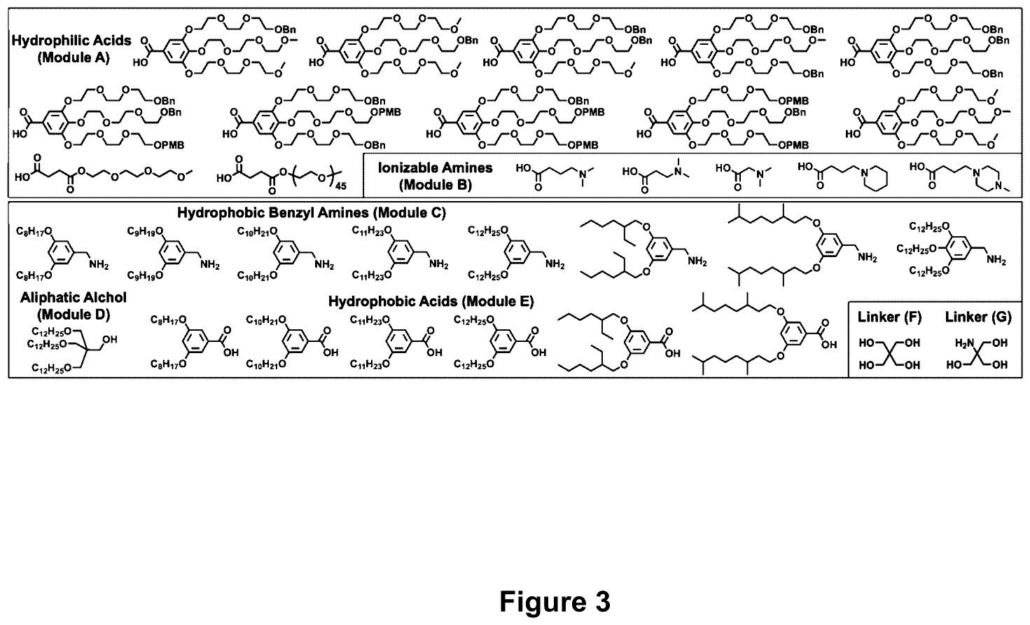

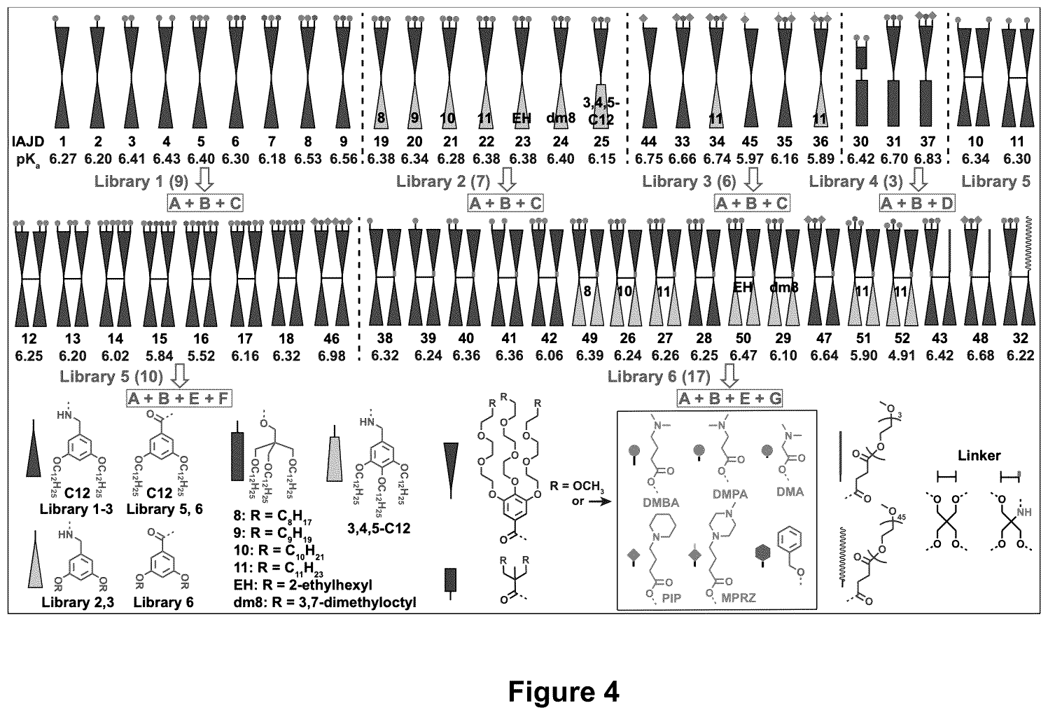

Accelerated Modular-Orthogonal Synthesis of Six Libraries of IAJDs: Modular-orthogonal methodologies were employed for the synthesis of sequence-defined amphiphilic Janus glycodendrimers (JGDs). Accelerated modular-orthogonal methodologies (FIG. 3 and FIG. 4) for the synthesis of single-single (a single hydrophobic combined with a single hydrophilic dendrons), twin-twin (two identical hydrophobic and two identical hydrophilic dendrons), and hybrid twin-mix (two identical hydrophobic and two different hydrophilic dendrons), rely on related but improved and accelerated synthetic principles originally employed for the synthesis of sequence-defined JGDs. Two different orthogonal protective groups, 4-methoxybenzyl ether and benzyl ether, were employed in this methodology (Horita, et al., Tetrahedron 1986, 42, 3021-3028). The six libraries synthesized are schematically shown in FIG. 4. Four of these libraries are based on single-single IAJDs. Nine IAJDs are available in library 1, seven in library 2, six in library 3 and three in library 4. Library 5 is based on the twin-twin IAJDs generated from IAJD1 to IAJD9 of library 1 and JAJD33 of library 3. Library 6 contains 17 hybrid twin-mix IAJDs selected from all libraries. The selection process was determined by the activity in vivo and in vitro of their single-single components. The hydrophilic parts of these IAJDs contain sequence-defined compositions based on the dimethylaminobutanoate (DMBA), dimethylaminopropanoate (DMPA), dimethylaminoacetate (DMA), piperidinebutanoate (PIP) and methylpiperazinebutanoate (MPRZ) ionizable amines (FIG. 3). They were selected based on the pKₐ of their corresponding ionizable lipids available in the literature (Ramishetti, et al., Adv. Mater. 2020, 32, 1906128; Kim, et al., Sci. Adv. 2021, 7, eabf4398). The symbols employed in the schematic representation of these sequence-defined IAJDs are shown in the square from the bottom part of FIG. 4. A benzyl ether group, marked in red, was employed to isolate the ionizable amines and construct different sequences in the hydrophilic part of the IAJDs. Aromatic groups such as benzyl ethers are suggested to interact as hydrogen-bond acceptors in molecular recognition with cations including ammonium groups both in biology and in synthetic supramolecular chemistry (Levitt, et al., Philos. Trans. R. Soc., A. 1993, 345, 105-112; Perutz, et al., J. Am. Chem. Soc. 1986, 108, 1064-1078; Loewenthal, et al., J. Mol. Biol. 1992, 224, 759-770; Zacharias, et al., Trends Pharmacol. Sci. 2002, 23, 281-287; Gallivan, et al., Proc. Natl. Acad. Sci. U.S.A. 1999, 96, 9459-9464; Dougherty, Science 1996, 271, 163-168; Dougherty, Acc. Chem. Res. 2013, 46, 885-893; Ma, et al., Chem. Rev. 1997, 97, 1303-1324; Dougherty, et al., Science 1990, 250, 1558-1560). This cation-π interaction is weaker than traditional H-bonds (about 3 kcal/mol) and therefore, can mediate a dynamic control of the pKₐ of the ionizable amines. In the protonated state of the amine, it can increase its pKₐ while in the non-protonated state it may facilitate an interaction of the benzyl ether with the nucleic bases of the mRNA to enhance co-assembly, or segregate in the hydrophobic part of the DNPs. This cation-π interaction has not been used previously in delivery vectors for mRNA. The hydrophobic parts of the IAJDs contains both linear and branched alkyl groups of different length (FIG. 3 and FIG. 4). The hydrophilic acid components of these IAJDs are shown in FIG. 3 (Module A); while their synthesis is described elsewhere herein. The structures of the hydrophobic benzyl amines are shown in FIG. 3 (Module C). Their synthesis is also described elsewhere herein. Combining in an orthogonal way all these modules as illustrated in FIG. 4 provides, in an accelerated way, the 6 libraries of IAJDs. The synthesis of the twin-twin part of the hydrophobic IAJDs is described elsewhere herein. The detailed structures of all libraries of IAJDs together with their pKₐ values and short names are presented in FIG. 12 (libraries 1,2,3,4), FIG. 13 (library 5), and FIG. 14 (library 6).

In Vitro Transfection Activity of DNPs in Human Embrionic Kidney (HEK) 293T Cells: HEK293T cells were seeded into 96-well plates (20,000 cells/well/200 μL) and cultured for 24 h at 37° C. in 5% CO2 complete cell culture media. Screening experiments were performed with nonoptimized DNPs containing naked nucleoside-modified mRNA (Karikó, et al., Immunity 2005, 23, 165-175; Pardi, et al., Methods Mol. Biol. 2013, 969, 29-42), of molar mass 664,341 encoding firefly luciferase (mRNA-Luc). A constant concentration of mRNA-Luc of 125 ng/well was used. The transIT (TransIT-mRNA transfection Kit from Mirus Bio) and MC3-based LNPs (MC3: DLin-MC3-DMA, which is the FDA approved LNP for mRNA delivery) were used as positive controls for cell transfection at a concentration of mRNA-Luc of 125 ng/well, identical to that of the tested DNPs. Subsequently cells were cultured for 24 h, the medium was aspirated under reduced pressure by a glass pipette and cells were lysed with 30 μL/well of cell culture lysis reagent (Promega). The luminescence intensity corresponding to the luciferase protein expressed was characterized and analyzed.

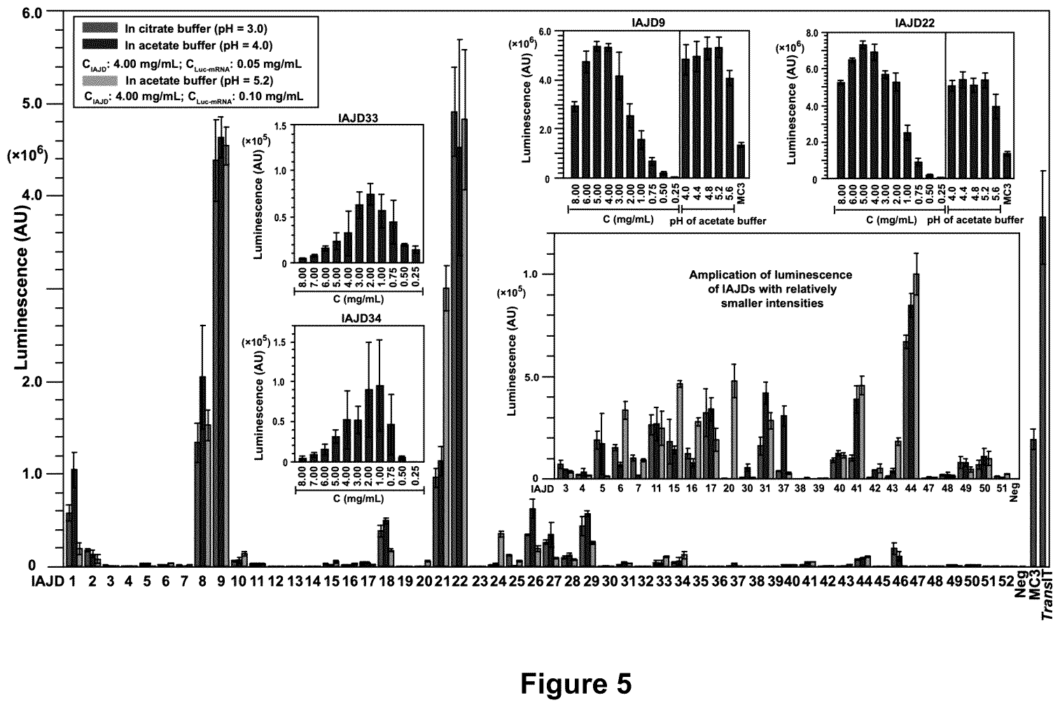

All 52 IAJDs from FIG. 4 were co-assembled by injection with mRNA in DNPs and were used in these experiments. Table 1 through Table 5 (vida infra) summarize the conditions employed for the preparation of the DNPs, their diameter (D, in nm) and polydispersity (PDI) as obtained by DLS experiments and the luminescence results. Each experiment was performed at least three times (FIG. 5). Out of 52 DNPs, 42 (81%) showed activity in vitro (FIG. 5). Without any optimization two of them DNP9 and DNP 22 display higher activity than the positive controls MC3 and TransIT, while four of them DNP8, DNP9, DNP21 and DNP22 show higher activity than the most commonly used positive control MC3.

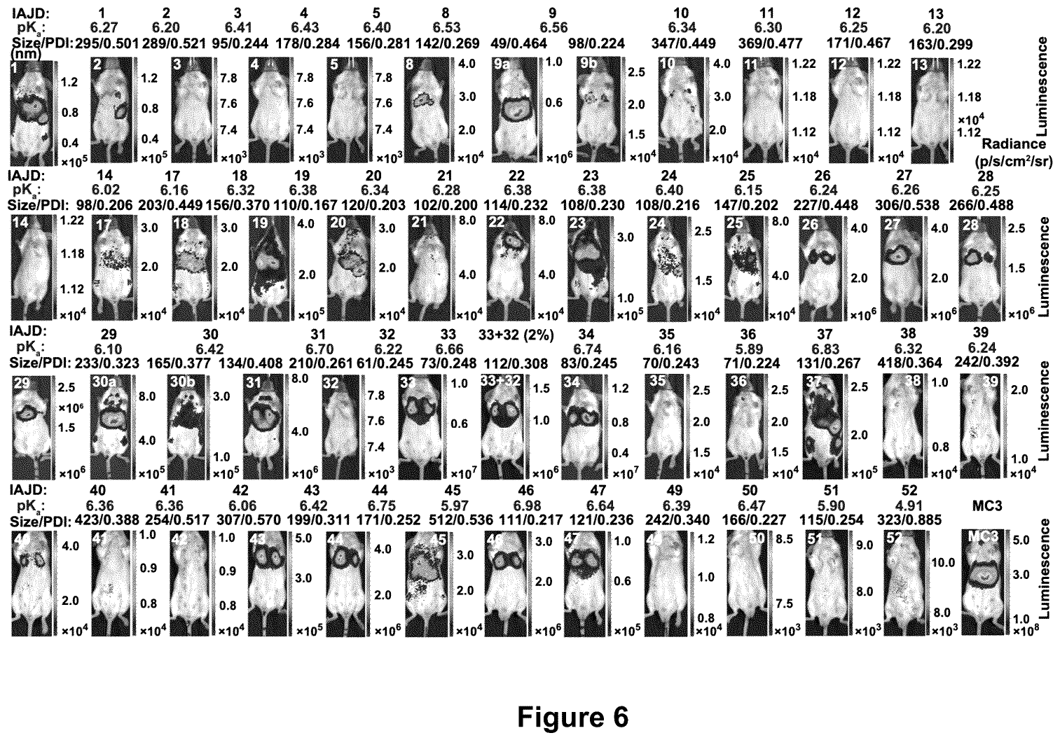

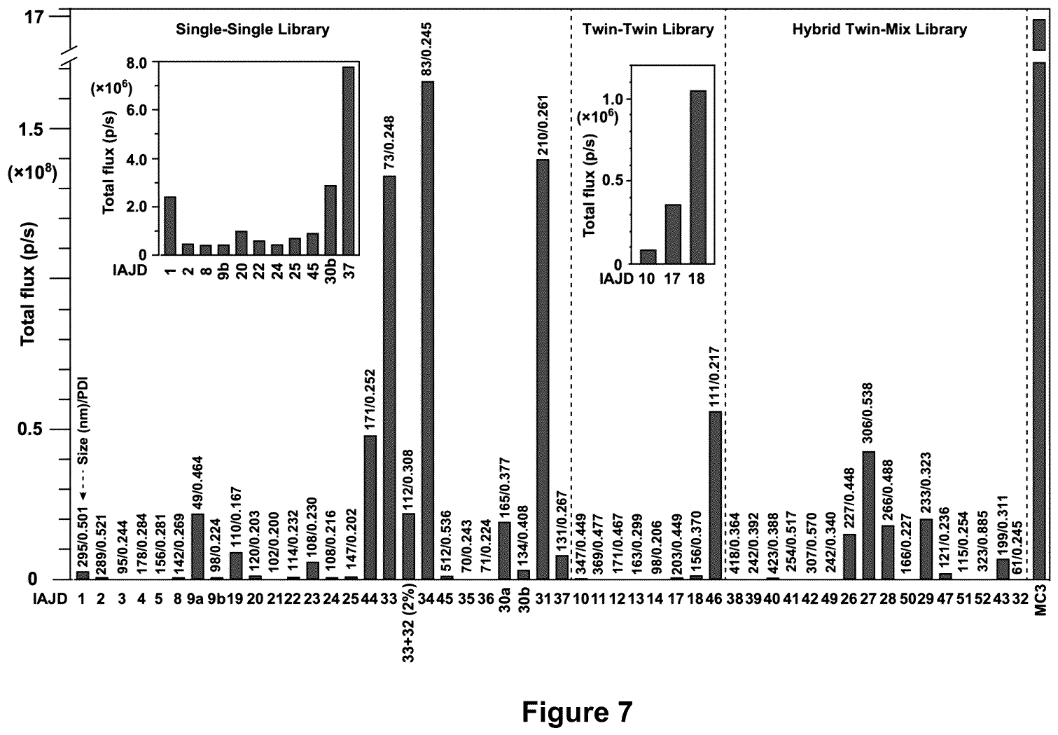

In vivo mRNA Delivery in Mice with DNPs: Six to eight weeks old female mice were used in these experiments. Four to seven hours after injection with 100 μL solution of DNP encapsulated with 10 μg of mRNA-Luc the mice were imaged 10 min after intraperitoneal injection with D-Luciferin, 15 mg/mL at 10 μL/g of body weight. The exposure time was 1 min. For imaging of the organs, mice were sacrified, the organs were immediately collected and bioluminescence imaging was performed. Table 7 (vida infra) summarizes all DNPs injection assembly data including D in nm and PDI determined by DLS together with the resulting results obtained in vivo. FIG. 6 summarizes all mice experiments including the D in nm and PDI of DNPs (both in black on top of the mice image), pKₐ values of the corresponding IAJDs (in blue also on top) of all compounds used for delivery. The results from FIGS. 6 and 7 provide a proof of concept for the one-component multifunctional sequence-defined ionizable amphiphilic Janus dendrimers delivery system for mRNA.

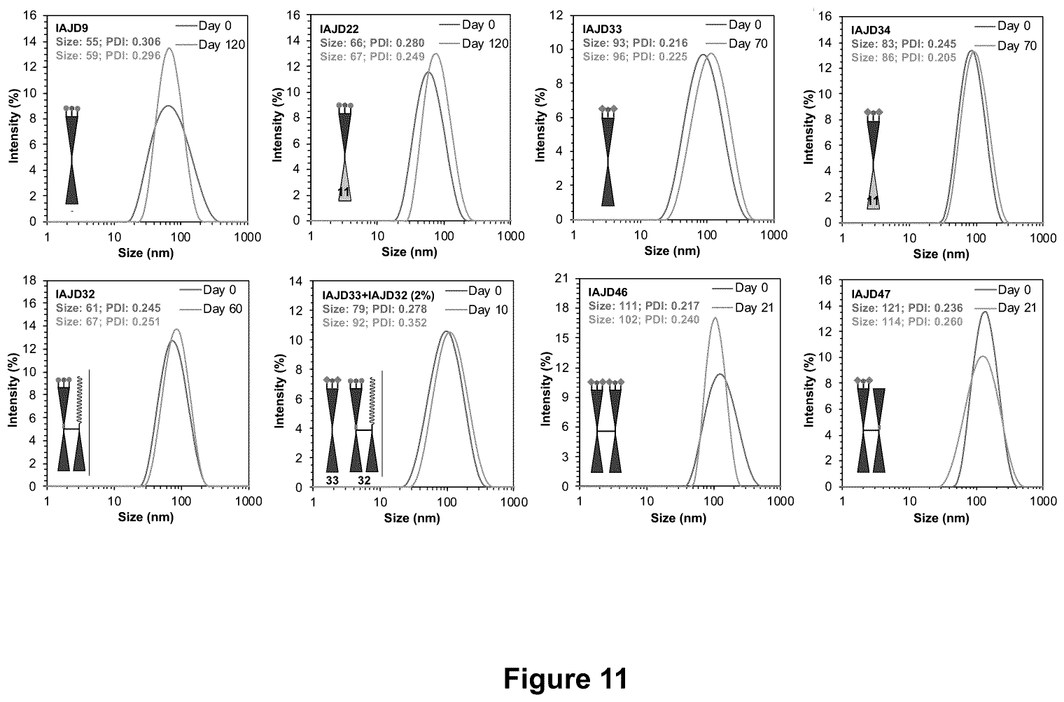

The summary of the results from FIG. 7 demonstrate that there seem to be no correlation between the activity of the DNPs in-vivo and the same experiments in vitro (FIG. 5) and that the most active IAJDs 33, 34 and 31 do not have the lowest pKₐ values. The results from FIG. 6 also demonstrate that there is quite a tolerance of the activity of DNPs to their diameters and polydispersity. Larger than 100 nm diameter seem to be as active as lower than 100 nm diameter DNPs (FIG. 6). However quantitative correlations for the dependences of activity with pKₐ and DNPs dimensions must be studied. The stability of the dimension of 40 DNPs was investigated as a 15 function of time at 5° C. Unoptimized experiments showed that the dimensions of 19 out of 40 DNPs were very stable after being stored at 5° C. for up to 120 days. Almost all these DNPs were assembled from IAJDs containing a benzyl ether in their hydrophilic part (Table 9, vida infra). A very interesting series of activity trends is observed when comparing the data from FIGS. 7 and 11. As shown in FIG. 11, the stability of the dimensions of the DNPs is excellent for up to 120 days when they are stored at 5° C. IAJD9, IAJD22, IAJD 33 and IAJD34 single-single compounds form remarkably stable DNPSs that also show high in-vivo activity (FIG. 7). IAJD46 is the twin-twin of IAJD33. However the activity of the DNP36 is about half that of DNP33 (FIGS. 6 and 7). The hybrid twin-mix IAJD47 is also based on the structure of IAJD33 or half of the structure of IAJD46. However, the activity of the DNP47 is much lower than both that of DNP33 and DNP46. Even more interesting is the comparison of single-single IAJD9 with the hybrid twin-mix IAJD32 (FIGS. 6 and 7).

FIG. 7 plots the results from FIG. 6. Without any optimization, out of the 52 DNPs investigated, 28 (54%) show activity in vivo. Two of them, DNP33 and DNP34 show very high activity in lung. Single-single IAJD9 derived DNP9 exhibits good activity (FIG. 7) and stability (FIG. 11). At the same time the corresponding hybrid twin-mix IAJD32 based DNP32 that is based on IAJD9 and a single PEG of degree of polymerization 45 (FIGS. 3 and 4) exhibits also excellent stability (FIG. 11) but it is completely inactive in mice (FIG. 7). In order to clarify this result, several co-assembled DNPs were prepared based on very active IAJDs and a small concentration of IAJD32. One example is the DNP assembled from IAJD33 and 2% IAJD32. (FIG. 11, bottom row, second from left) and FIG. 7. The stability of this combined DNP is excellent (FIG. 11) and is comparable with that of DNP32 assembled from IAJD32 alone. However, the in vivo activity of DNP co-assembled from IAJD33 with 2% IAJD32 is only a small fraction of the activity of the DNP33 (FIG. 7). This confirms the mechanism of the PEG enigma. Therefore, while insertion of a small fraction of PEG-conjugated to an IAJD can increase dramatically the stability of the resulting DNP it also decreases even more dramatically its activity in vivo. Incorporation of short oligooxyethylene fragments in the structure of single-single, twin-twin or even hybrid twin-mix IAJDs may serve to address this issue.

The Role of Ionizable Amine Concentration and Sequence on the Activity of the Corresponding DNPs: The binding activity of sugars located on the surface of the glycodendrimersomes (GDSs) assembled from amphiphilic Janus glycodendrimers (JGDs) toward sugar binding proteins increases by decreasing the concentration of the sugar in a sequence-defined process (Percec, et al., J. Am. Chem. Soc. 2013, 135, 9055-9077). This unexpected trend was explained by self-organization on the periphery of the glycodendrimersome of a morphology that facilitated higher binding activity between the sugar and the proteins at lower concentrations of sugar.

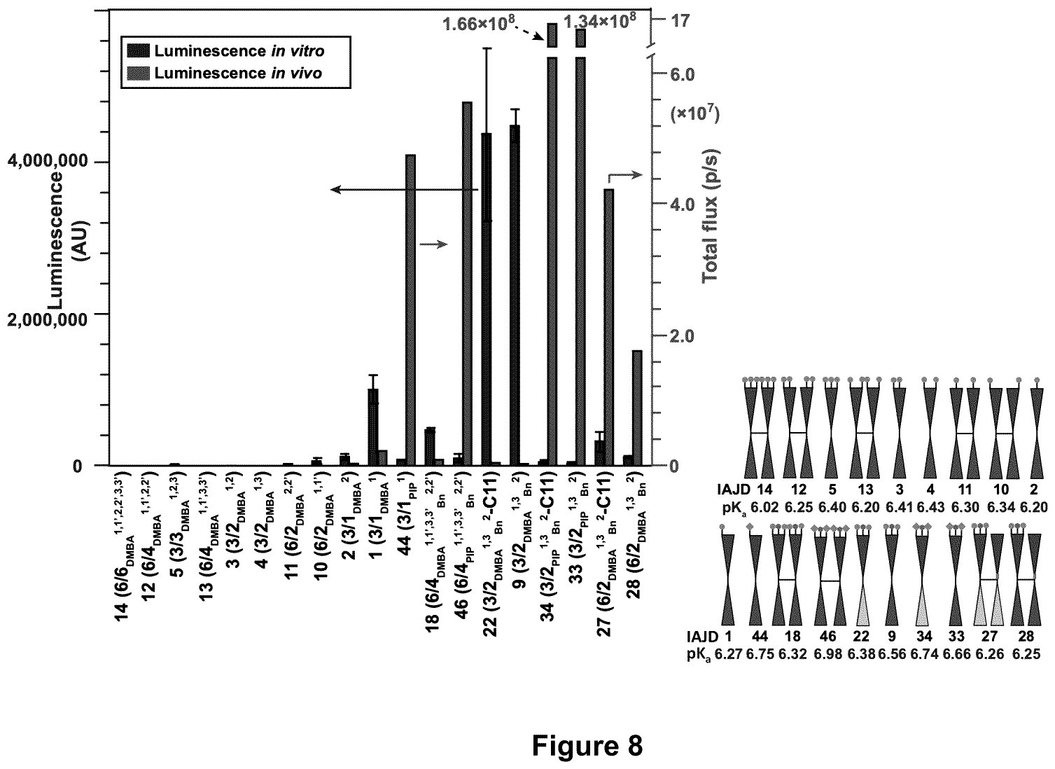

FIG. 8 plots representative activity data for the sequence-defined IAJDs derived DNPs both in vitro and in vivo experiments. Low or no activity was observed at high concentrations of ionizable amines in the structure of the IAJD and extremely high activities were observed at lower ionizable amine concentration in very specific sequences. Without discussing in great details, the results from FIG. 8, provide a mechanism to engineer activity of NDPs via the sequence and concentration of their ionizable amines. The change in activity observed in FIG. 8 is much higher than that observed in the case of sequence-defined dendrimersomes.

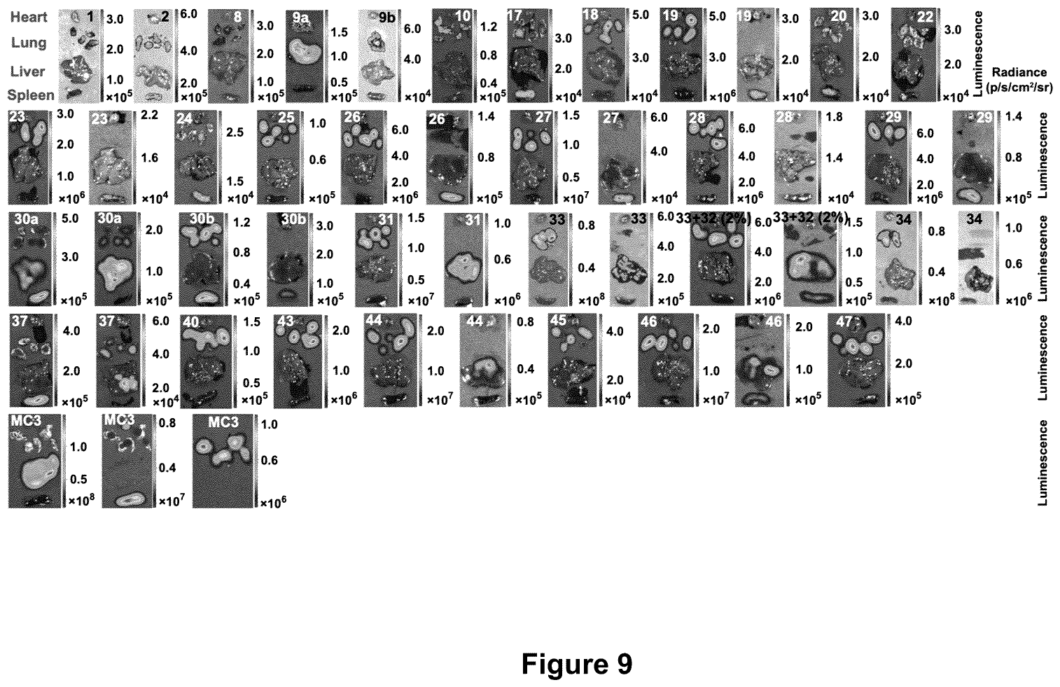

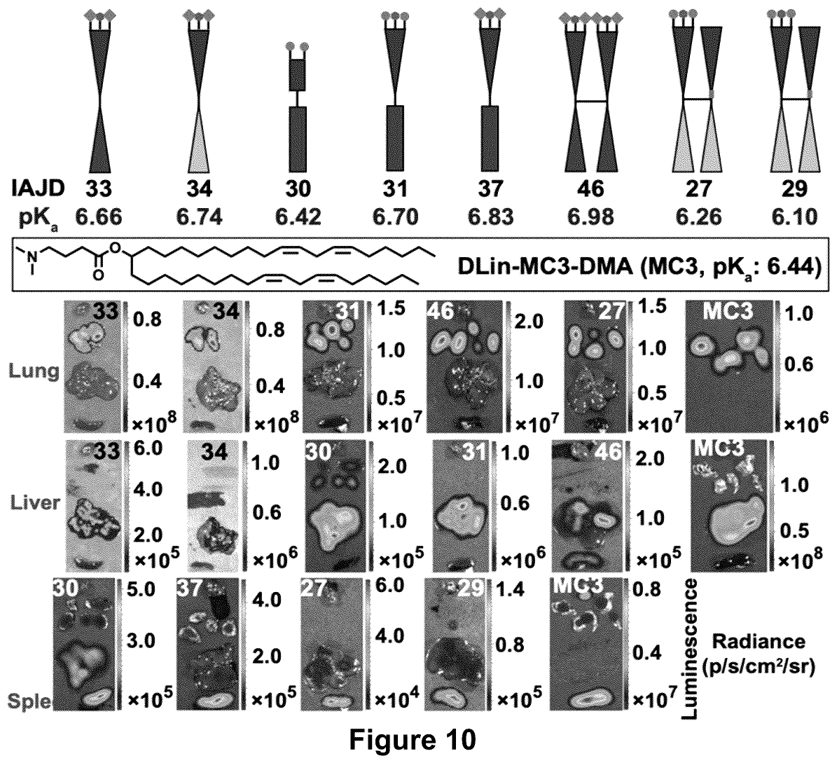

Some Comments On Potential Targeted Delivery of mRNA: FIG. 9 and FIG. 10 summarize representative organ delivery data selected from the experiments reported in FIG. 6. They illustrate the luminescence intensity reflecting delivery activity in heart, lung, liver and spleen as a function of the structure of the IAJD employed in the design of the structure of the DNP used in the delivery of mRNA. The highest luminescence is exhibited by single-single IAJD33 and IAJD34 forming DNPs (10⁸) in lung. The next higher is again based on the IAJD31, IAJD46 and IAJD27 (10⁷) based DNPs and is also in lung.

These lung activities are higher than the activity in lung of the control experiment of MC3 (FIG. 9). It is important to realize that organ activities from FIG. 10 are much higher than overall mice activities from FIGS. 7 and 9. The next higher activity is in liver (FIGS. 9 and 10). Without optimization, the luminescence in liver is 10⁶ for IAJD31, IAJD34 DNPs and 105 for IAJD30, IAJD33 and IAJD46 based DNPs. They are smaller than the values of MC3 that is 10′. The highest activity in spleen is for IAJD29, IAJD30, IAJD37 based DNPs (105) and IAJD27 based DNP (104). The control experiment with MC3 is 107 in spleen. These results indicate that single component IAJDs could provide a potential strategy to target different organs.

The design and accelerated modular-orthogonal synthesis of six libraries containing 52 multifunctional sequence-defined ionizable amphiphilic Janus dendrimers (IAJDs) are reported. All 52 IAJDs co-assemble with mRNA by simple injection in acidic buffers to generate dendrimersome nanoparticles (DNPs) with predictable dimensions and narrow polydispersity. The dimensions of these DNPs are stable for over 120 days at 5° C. Among the 52 DNPs encapsulating Luciferase-mRNA, 42 (81%) showed activity in vitro and four of them showed higher activity than the four component lipid nanoparticles obtained from the MC3 FDA approved control experiment. 28 (54%) DNPs displayed activity in vivo with two of them exhibiting higher activity than the MC3 control experiment in the lung. These unoptimized preliminary experiments provide proof of concept for the one-component multifunctional sequence-defined amphiphilic Janus dendrimer as an efficient delivery system for mRNA. This one-component delivery platform can be used to elucidate the mechanisms of encapsulation and release of mRNA from supramolecular virus-like assemblies and for the production of vaccines and drugs.

mRNA vaccines rely on a delivery system for the nucleic acid based on four-component ionizable lipid nanoparticles (LNPs), containing phospholipids, cholesterol for mechanical properties, PEG conjugated lipids for stability and ionizable amines.

In this report, the current four-component LNP delivery system is transformed into a simpler and more precise one-component multifunctional JD system, with highest activity mediated by a sequence-defined low concentration of ionizable amines.

A new class of JDs, named ionizable amphiphilic Janus dendrimers (IAJDs), was designed and synthesized. This concept provides the first attempt to elucidate the role of ionizable amine(s) (IA) and their ability to encapsulate and release mRNA as a function of concentration, constitutional isomerism, number of ionizable amines and their sequence by using IAJDs based DNPs as a delivery tool. The assembly of one-component DNPs relies on a simple injection method. This process is much simpler than the microfluidic device required to assemble four-component LNPs.

Six libraries containing 52 sequence-defined IAJDs were synthesized via an accelerated modular-orthogonal methodology.

Among the 52 DNPs, assembled without any optimization from IAJDs encapsulating Luciferase-mRNA, 45 (87%) showed activity in vitro with two of them (IAJD9 and 22) displaying higher activity than the positive controls, MC3 and transIT. 28 (54%) showed activity in vivo with two of them (IAJD33 and 34) exhibited very high activity in the lungs.

The size stability of 40 DNPs was tested. Unoptimized screening experiments showed that sizes of 19 out of 40 DNPs were very stable after storage at 5° C. for up to 120 days. Almost all these DNPs were assembled from IAJDs containing a benzyl ether in the hydrophilic part.

This work provides proof of concept for one-component IAJDs as efficient delivery systems for mRNA. They will be employed to produce vaccines and drugs by mRNA delivery.

The materials and methods employed in these experiments are now described.

Triethylene glycol monomethyl ether (TCI, 98%), triethylene glycol (Alfa Aesar, 99%), benzyl chloride (Alfa Aesar, 99%), 4-methoxybenzyl chloride (TCI, 98%), p-toluenesulfonyl chloride (Alfa Aesar, 98%), gallic acid (Chem Impex, anhydrous, ACS grade), triethyl orthoformate (TCI, 98+%), Amberlyst-15(H) (Alfa Aesar), 1-bromooctane (Aldrich, 99%), 1-bromononane (Lancaster, 99%), 1-bromoundecane (Aldrich, 99%), 1-bromododecane (Alfa Aesar, 99%), (rac)-3-(bromomethyl)heptane (Aldrich, 95%), (rac)-1-bromo-3,7-dimethyloctane (TCI, 93+%), palladium on activated carbon catalyst (Spectrum, 10 wt % loading), pentaerythritol (Aldrich, 98%), 4-(dimethylamino)butyric acid hydrochloride (Alfa Aesar, 98%), 3-(dimethylamino)propionic acid hydrochloride (TCI, 98%), N,N-dimethylglycine hydrochloride (Acros, 99%), piperidine (Beantown Chemical, 99%), 1-methylpiperazine (Alfa Aesar, 98%), citrate buffer (100 mM, pH 3.0, TEKnova), tris buffer (1 M, Thermo Fisher Scientific) and other reagents and solvents were obtained from commercial sources and were used as received. 4-(Dimethylamino)pyridinium 4-toluenesulfonate (DPTS) was prepared according to a literature procedure. 2-Chloro-4,6-dimethoxy-1,3,5-triazine (CDMT) was prepared according to a literature procedure. Citrate buffer (100 mM, pH 3.0, TEKnova) CH₂Cl₂ (DCM) was dried over CaH2 and freshly distilled before use.

The purity and structural identity of final products and intermediates was determined by a combination of techniques that include thin-layer chromatography (TLC), high-pressure liquid chromatography (HPLC), ¹H and ¹³C NMR, and matrix assisted laser desorption ionization-time of flight (MALDI-TOF) mass spectrometry.

¹H and ¹³C NMR spectra were recorded at 400 MHz and 101 MHz, on a Bruker NEO (400 MHz) NMR spectrometer with autosampler, and 500 MHz and 126 MHz respectively, on a Bruker DRX (500 MHz) NMR spectrometer. All NMR spectra were measured at 23° C. in CDCl₃. Residual protic solvent of CDCl₃ (¹H, δ 7.26 ppm; ¹³C, δ 77.16 ppm), and tetramethylsilane (TMS, δ ppm) were used as the internal reference in the ¹H- and ¹³C-NMR spectra. The absorptions are given in wavenumbers (cm¹). NMR spectra were analyzed and exported by TopSpin 4.07 (Bruker) or MNova 14.

The determination of the purity of molecules by high-pressure liquid chromatography (HPLC) was carried out using Shimadzu LC-20AD high-performance liquid chromatograph pump, a PE Nelson Analytical 900 Series integration data station, a Shimadzu SPD-10A VP (UV-vis, =254 nm) and three AM gel columns (a guard column, two 500 Å, 10 pm columns). THF with 5% of NEt₃ was used as solvent at the oven temperature of 23° C. Detection was done by UV absorbance at 254 nm.

Matrix Assisted Laser Desorption Ionization-Time of Flight (MALDI-TOF) Mass Spectrometry was performed on a PerSeptive Biosystem-Voyager-DE (Framingham, MA) mass spectrometer equipped with nitrogen laser (337 nm) and operating in linear mode. Internal calibration was performed using Angiotensin II and Bombesin as standards. The analytical sample solution was prepared by mixing the THF solution of the sample (5-10 mg/mL) and THF solution of the matrix (2,5-dihydroxybenzoic acid, 10 mg/mL) with a 1/5 (v/v) ratio. The prepared sample solution (0.5 μL) was loaded on the MALDI plate and dried at 23° C. before the plate was inserted into the vacuum chamber of the instrument. The laser intensity and voltages applied were adjusted depending on the molecular weight and the nature of each analyzed compound.

Dynamic Light Scattering (DLS) for the DNPs was performed in buffer with a Malvern Instruments particle sizer (Zetasizer Nano S, Malvern Instruments, UK) equipped with 4 mW He—Ne laser 633 nm and avalanche photodiode positioned at 1750 to the beam and temperature-controlled cuvette holder. Instrument parameters were determined automatically along with measurement times.

pKₐ Measurements of Individual IAJD Molecules: IAJD Molecules were dissolved in ethanol (Sat. with NaCl) at a concentration of 1.5 mg/mL in a volume of 3 mL. 0.1 M HCl aqueous solution was added in increments of 7.5 μL, with the resulting pH measured using an Hach H170 pH meter. pKₐ was calculated using half equivalence point titration.

Formulation of DNPs Containing mRNA-Luc: IAJDs were dissolved in ethanol with various initial concentrations (5-160 mg/mL). Nucleoside-modified mRNA encoding firefly luciferase (mRNA-Luc) was dissolved in water with various initial concentrations (1-4 mg/mL). 12.5 μL of mRNA solution was placed into a clean RNAs free eppendorf (1.5 mL) and mixed with 463 μL of citrate buffer (10 mM, pH 3.0)/acetate buffer (10 mM). For encapsulation of mRNA into IAJDs, 25 μL of IAJDs of the ethanol stock solution was rapidly injected into mRNA solution in citrate or acetate buffer followed by vortex for 5 seconds.

Dialysis of DNPs: The 0.5 mL DNP solution was dialyzed against 10 mM tris buffer (pH 7.4) or 1×PBS buffer (pH 7.4) for 2 h in 3,500-14,000 molecular weight cut-off dialysis tube (Spectrum Medical Instruments Inc. Spectra/Por molecular porous membrane tubing Flat Width: 45 mm; Diameter: 29 mm & Vol/length: 6.4 mL/cm).

In vitro mRNA Delivery: Human embryonic kidney (HEK) 293T cells (American Type Culture Collection) were cultured in Dulbecco's modified Eagle's medium (DMEM) supplemented with 10% inactivated fetal bovine serum (FBS) (Gemini Bio-Products), 2 mM L-glutamine and 100 U/mL penicillin/streptomycin (Life Technologies) (complete medium). For in vitro screening experiments, HEK293T cells were seeded into 96-well plates (20,000 cells/well/200 μL) and cultured for 24 h in at 37° C. and 5% CO2 in complete media. Cell were transfected with IAJDs with encapsulated naked nucleoside-modified mRNA encoding firefly luciferase (mRNA-Luc) with fixed concentration of mRNA-Luc, 125 ng per well. The transIT (TransIT®-mRNA Transfection Kit, Mirus Bio) was used as a positive control for cell transfection, accordingly per manufacturer protocol and added to cells at the concentration of mRNA-Luc, 125 ng per well, the same as for tested particles. Cells were further cultured for 24 h, then medium was aspirated, and cells were lysed with 30 μL/well of cell culture lysis reagent (Promega). For the characterization of luminescence intensity, 2.5 μL of the lysed cells was mixed with 10 μL of luciferase assay substrate and luminescence was analyzed using a MiniLumat LB 9506 luminometer (Berthold/EG&G; Wallac).

In vivo mRNA Delivery: Female BALB/c mice aged 6-8 weeks were purchased from Charles River Laboratories. 100 μL of buffer solution containing IAJDs with encapsulated 10 μg of mRNA-Luc was retro-orbitally injected into mice under Isoflurane-anesthesia. After 4-7 h post injection mice were imaged on a Perkin Elmer IVIS Spectrum CT system 10 minutes after intraperitoneal injection of D-Luciferin, 15 mg/mL (Regis Technologies) at 10 μL/g body weight. The exposure time was set to 1 min using medium binning (binning=8). For IVIS imaging of the organs, mice were sacrificed, then the organs were immediately collected, and bioluminescence imaging was performed.

### Synthesis of Hydrophilic Acids

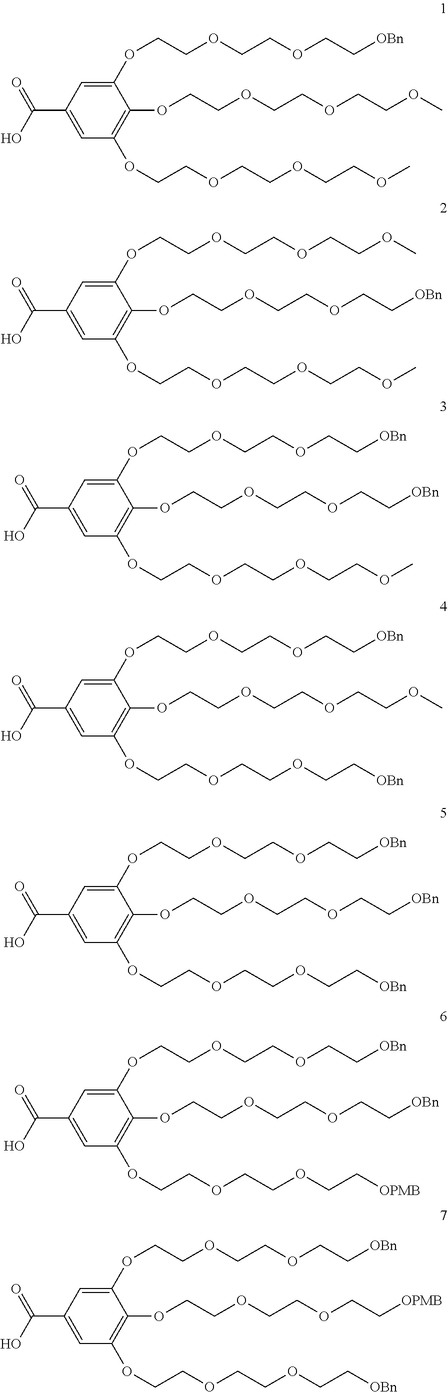

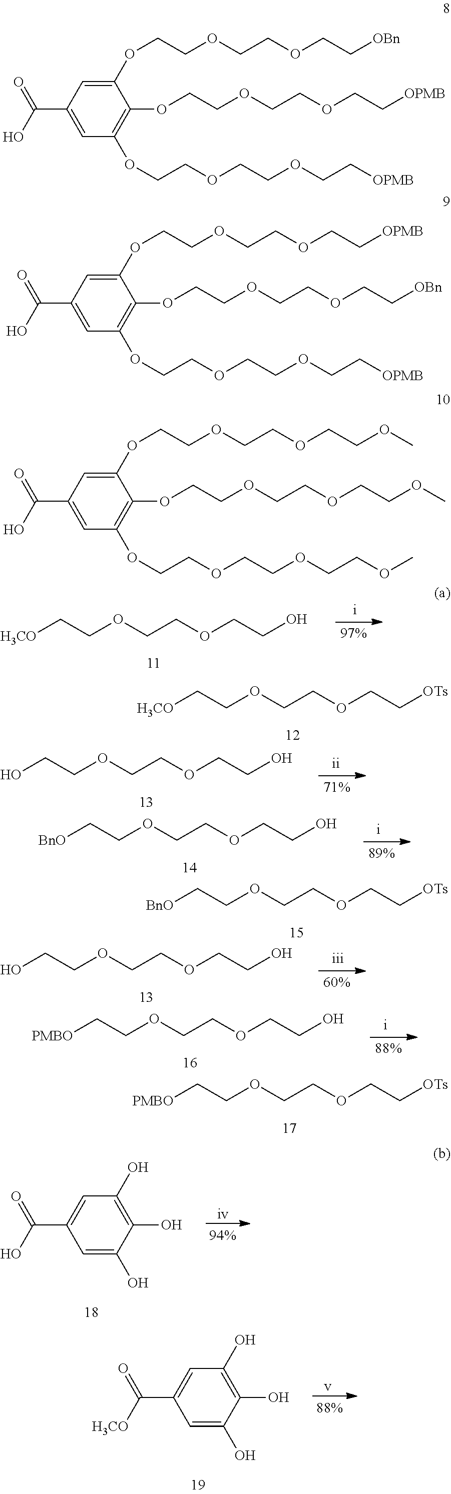

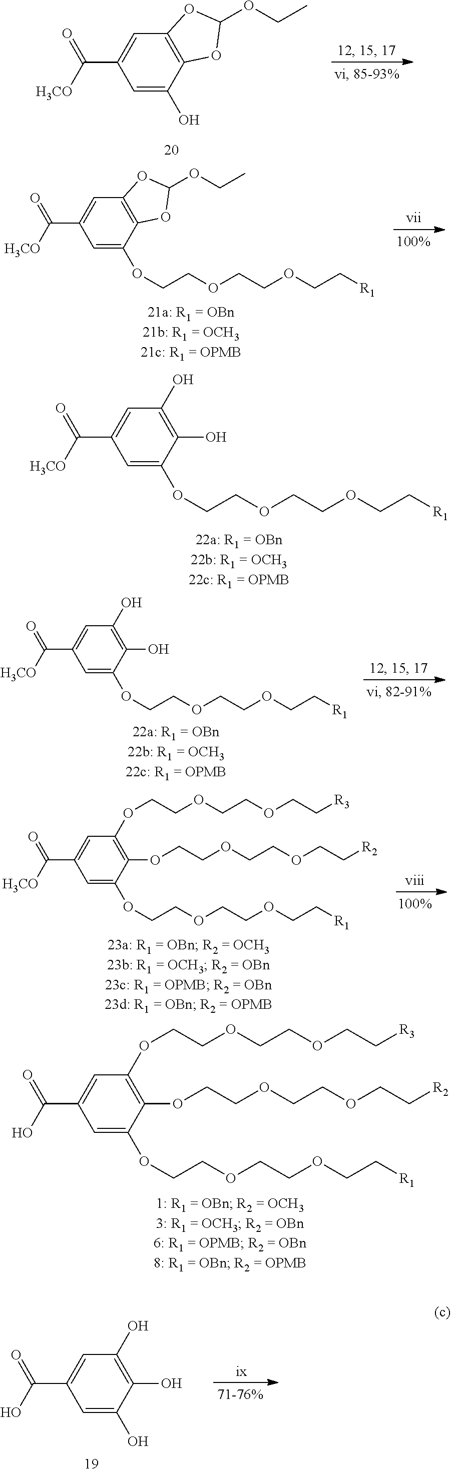

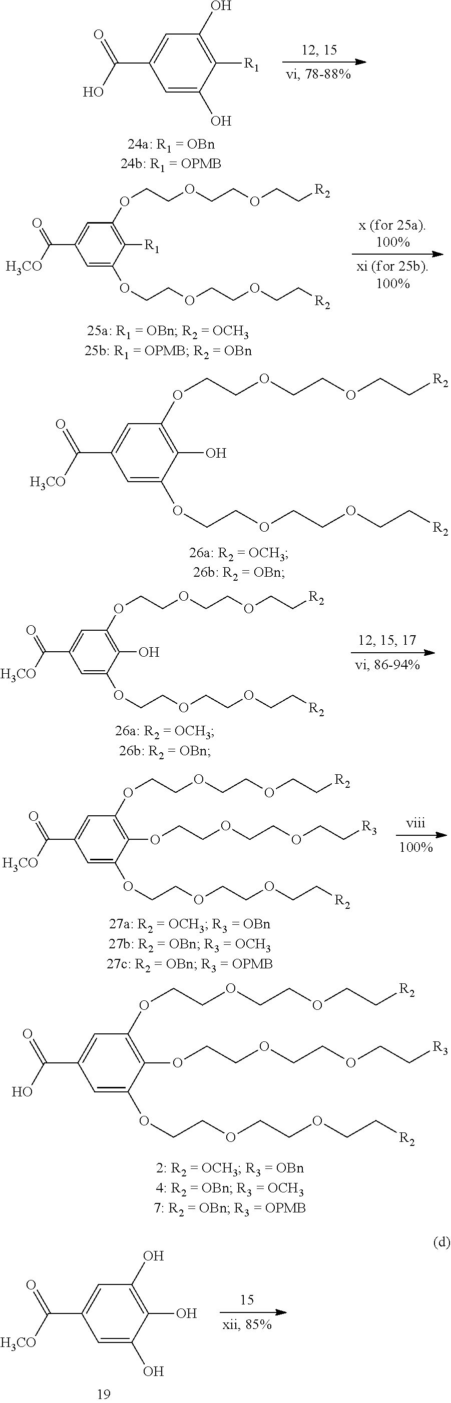

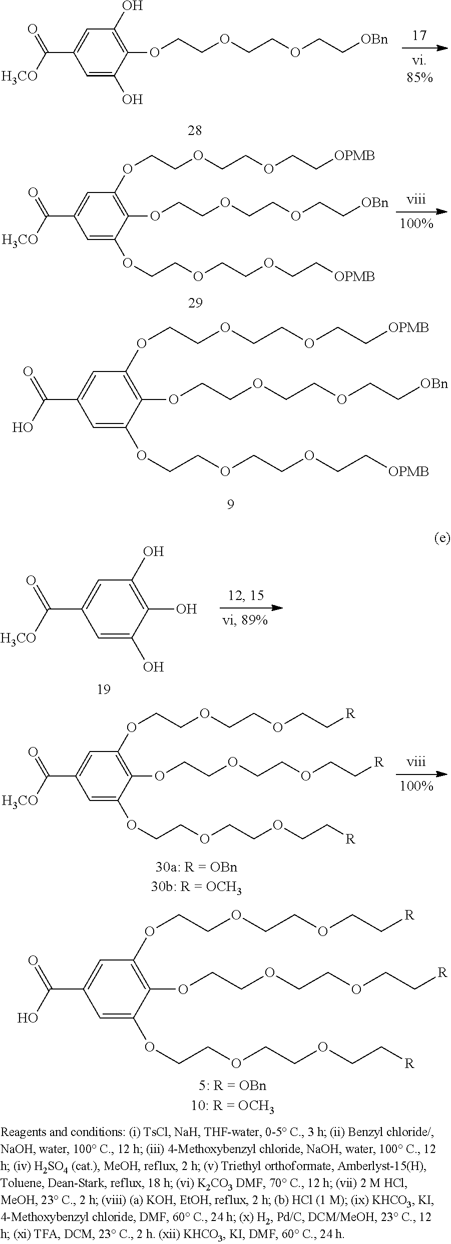

The synthesis of 2-(2-(2-methoxyethoxy)ethoxy)ethyl 4-methylbenzenesulfonate (11), 2-(2-(2-(benzyloxy)ethoxy)ethoxy)ethan-1-ol (14) and 2-(2-(2-(benzyloxy)ethoxy)ethoxy)ethyl 4-methylbenzenesulfonate (15) was adapted from literature procedures previously reported. The procedures are optimized to increase the yields of compounds 11, 14 and 15 to 78.02 g, 20.21 g, and 28.81 g, respectively. The synthesis of methyl 2-ethoxy-7-hydroxybenzo[d][1,3]dioxole-5-carboxylate (20) and methyl 4-(benzyloxy)-3,5-dihydroxybenzoate (24a) was adapted from literature procedures (Wang, et al., J. Am. Chem. Soc. 2020, 142, 9525-9536). Methyl 3,4,5-tris(2-(2-(2-(benzyloxy)ethoxy)ethoxy)ethoxy) benzoate (30a), methyl 3,4,5-tris(2-(2-(2-methoxyethoxy)ethoxy)ethoxy)benzoate (30b), 3,4,5-tris(2-(2-(2-(benzyloxy)ethoxy)ethoxy)ethoxy)benzoic acid (5) and 3,4,5-tris(2-(2-(2-methoxyethoxy)ethoxy)ethoxy)benzoic acid (10) were prepared according to literature procedures (Percec, et al., Science 2010, 328, 1009-1014; Percec, et al., J. Am. Chem. Soc. 2013, 135, 4129-4148).

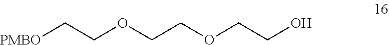

2-(2-(2-((4-Methoxybenzyl)oxy)ethoxy)ethoxy)ethan-1-ol (16). Triethylene glycol (13, 34.60 g, 229.75 mmol, 4 equiv) was added to 120 mL NaOH aqueous solution (50%). The mixture was stirred at 23° C. for 30 min. Then 4-methoxybenzyl chloride (9.00 g, 57.47 mmol, 1 equiv) was added and the mixture was allowed to stir at 100° C. for 24 h. The reaction mixture was cooled to 23° C., followed by diluting with water (80 mL) and extracting with diethyl ether (60 mL×3). The organic phase was dried over anhydrous magnesium sulfate (MgSO₄) and filtered. The filtrate was concentrated and purified by column chromatography (SiO₂) with ethyl acetate (EtOAc)/hexane=1/1 as the eluent to give the title compound as a brown-red oil (9.82 g, 60%).

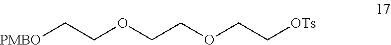

2-(2-(2-((4-Methoxybenzyl)oxy)ethoxy)ethoxy)ethyl 4-methylbenzenesulfonate (17). Compound 16 (8.31 g, 30.74 mmol, 1 equiv) and NaOH (1.84 g, 46.11 mmol, 1 equiv) were dissolved in the mixture of THF and water (1/1, 40 mL) and the mixture was cooled to 0° C. 4-Toluenesulfonyl chloride (TsCl, 5.86 g, 30.74 mmol, 1 equiv) dissolved in 20 mL THF was added to the mixture over 30 min at 0° C. The reaction mixture was allowed to stir at 0-5° C. for 2 h. The reaction mixture was poured into the ice-water bath (100 mL), and the mixture was extracted by DCM (50 mL×3). The organic phase was dried over anhydrous MgSO₄ and filtered. The filtrate was concentrated and purified by column chromatography (SiO₂) with EtOAc/hexane=1/1 as the eluent to give the title compound as a brown-red oil (11.45 g, 88%).

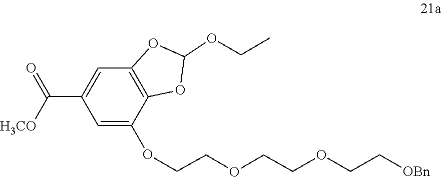 [SMILES: CCOC1Oc2cc(C(=O)OC)cc(OCCOCCOCCOCc3ccccc3)c2O1]

Methyl 7-(2-(2-(2-(benzyloxy)ethoxy)ethoxy)ethoxy)-2-ethoxybenzo-[d][1,3]dioxole-5-carboxylate (21a). Methyl 2-ethoxy-7-hydroxybenzo[d][1,3]dioxole-5-carboxylate (20, 0.80 g, 3.33 mmol, 1 equiv) and K₂CO₃ (1.38 g, 10.00 mmol, 3 equiv) were stirred in dry DMF (50 mL). 2-(2-(2-(benzyloxy)ethoxy)ethoxy)ethyl 4-methylbenzenesulfonate (15, 1.58 g, 4.00 mmol, 1.2 equiv) was added and the mixture was stirred at 70° C. under N₂ atmosphere for 12 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with hexane/EtOAc=1/1 as the eluent to give the title compound as a light-yellow oil (1.43 g, 93%).

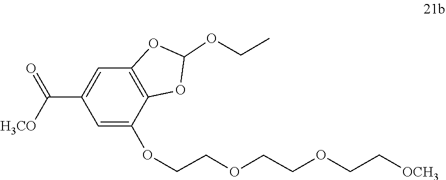 [SMILES: CCOC1Oc2cc(C(=O)OC)cc(OCCOCCOCCOC)c2O1]

Methyl 2-ethoxy-7-(2-(2-(2-methoxyethoxy)ethoxy)ethoxy)benzo-[d][1,3]dioxole-5-carboxylate (21b). Compound 20 (5.00 g, 20.82 mmol, 1 equiv) and K₂CO₃ (8.63 g, 62.46 mmol, 3 equiv) were stirred in dry DMF (70 mL). Compound 12 (7.95 g, 24.98 mol, 1.2 equiv) was added and the mixture was stirred at 70° C. under N₂ atmosphere for 12 h. The reaction mixture was cooled to 23° C. and DMF was removed under reduced pressure. Then water (50 mL) was added, and the mixture was extracted by DCM (50 mL×3). The organic phase was dried over anhydrous MgSO₄ and filtered. The filtrate was concentrated and purified by column chromatography (SiO₂) with hexane/EtOAc=1/2 as the eluent to give the title compound as a light-yellow oil (6.23 g, 86%).

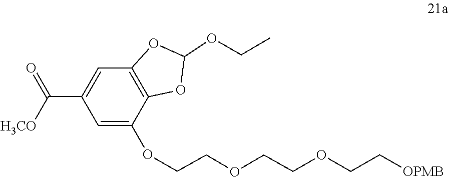

Methyl 2-ethoxy-7-(2-(2-(2-((4-methoxybenzyl)oxy)ethoxy)ethoxy)ethoxy)-benzo[d][1,3]dioxole-5-carboxylate (21c). Compound 20 (0.70 g, 2.91 mmol, 1 equiv) and K₂CO₃ (1.21 g, 8.73 mmol, 3 equiv) were stirred in dry DMF (30 mL). Compound 17 (1.36 g, 3.20 mmol, 1.1 equiv) was added and the mixture was stirred at 70° C. under N₂ atmosphere for 12 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with hexane/EtOAc=1/1 as the eluent to give the title compound as a light-yellow oil (1.22 g, 85%).

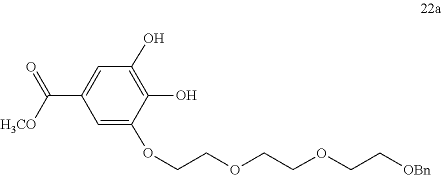 [SMILES: COC(=O)c1cc(O)c(O)c(OCCOCCOCCOCc2ccccc2)c1]

Methyl 3-(2-(2-(2-(benzyloxy)ethoxy)ethoxy)ethoxy)-4,5-dihydroxybenzoate (22a). Compound 21a (0.97 g, 2.10 mmol) was dissolved in 20 mL MeOH. Then HCl (2 M, 10.0 mL, 20.00 mmol) was added. The mixture was stirred at 23° C. for 2 h. The reaction mixture was extracted by DCM (20 mL×3) and the organic phase was dried over anhydrous MgSO₄. Filtration and evaporation of the solvent yielded the title compound as a light-yellow oil (0.85 g, 100%).

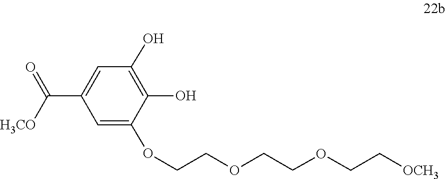 [SMILES: COCCOCCOCCOc1cc(C(=O)OC)cc(O)c1O]

Methyl 3,4-dihydroxy-5-(2-(2-(2-methoxyethoxy)ethoxy)ethoxy)benzoate (22b). Compound 21b (5.36 g, 13.88 mmol) was dissolved in 30 mL MeOH. Then HCl (2 M, 30.0 mL, 60.00 mmol) was added. The mixture was stirred at 23° C. for 2 h. The reaction mixture was extracted by DCM (30 mL×3) and the organic phase was dried over anhydrous MgSO₄. Filtration and evaporation of the solvent yielded the title compound as a light-yellow oil (4.58 g, 100%).

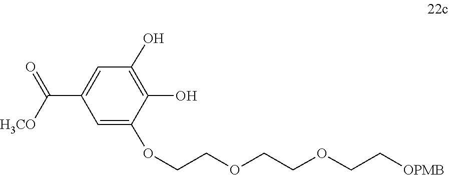

Methyl 3,4-dihydroxy-5-(2-(2-(2-((4-methoxybenzyl)oxy)ethoxy)ethoxy)ethoxy)-benzoate (22c). Compound 21c (0.90 g, 1.83 mmol) was dissolved in 10 mL MeOH. Then HCl (2 M, 4.0 mL, 8.00 mmol) was added. The mixture was stirred at 23° C. for 2 h. The reaction mixture was extracted by DCM (20 mL×3) and the organic phase was dried over anhydrous MgSO₄. Filtration and evaporation of the solvent yielded the title compound as a light-yellow oil (0.80 g, 100%).

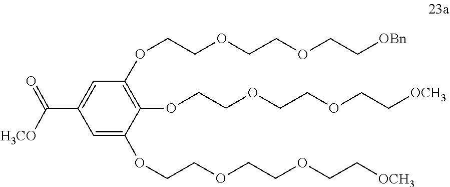 [SMILES: COCCOCCOCCOc1cc(C(=O)OC)cc(OCCOCCOCCOCc2ccccc2)c1OCCOCCOCCOC]

Methyl 3-(2-(2-(2-(benzyloxy)ethoxy)ethoxy)ethoxy)-4,5-bis(2-(2-(2-methoxyethoxy)ethoxy)ethoxy) benzoate (23a). Compound 22a (0.84 g, 2.07 mmol, 1 equiv) and K₂CO₃ (1.72 g, 12.42 mmol, 6 equiv) were stirred in dry DMF (30 mL). Compound 12 (1.45 g, 4.55 mol, 2.2 equiv) was added and the mixture was stirred at 70° C. under N₂ atmosphere for 12 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with EtOAc/MeOH=30/1 as the eluent to give the title compound as a yellow oil (1.19 g, 82%).

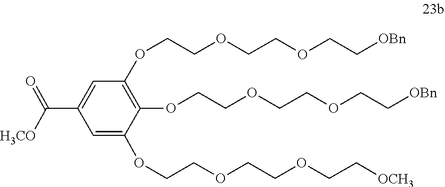 [SMILES: COCCOCCOCCOc1cc(C(=O)OC)cc(OCCOCCOCCOCc2ccccc2)c1OCCOCCOCCOCc1ccccc1]

Methyl 3,4-bis(2-(2-(2-(benzyloxy)ethoxy)ethoxy)ethoxy)-5-(2-(2-(2-methoxyethoxy)ethoxy)ethoxy) benzoate (23b). Compound 22b (4.00 g, 12.11 mmol, 1 equiv) and K₂CO₃ (10.04 g, 72.66 mmol, 6 equiv) were stirred in dry DMF (70 mL). Compound 15 (10.98 g, 27.83 mol, 2.3 equiv) was added and the mixture was stirred at 70° C. under N₂ atmosphere for 12 h. The reaction mixture was cooled to 23° C. and DMF was removed under reduced pressure. Then water (50 mL) was added, and the mixture was extracted by DCM (50 mL×3). The organic phase was dried over anhydrous MgSO₄ and filtered. The filtrate was concentrated and purified by column chromatography (SiO₂) with EtOAc/MeOH=50/1 as the eluent to give the title compound as a light-yellow oil (8.42 g, 90%).

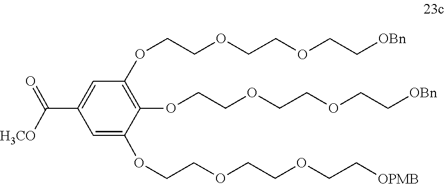

Methyl 3,4-bis(2-(2-(2-(benzyloxy)ethoxy)ethoxy)ethoxy)-5-(2-(2-(2-((4-methoxybenzyl)oxy)ethoxy) ethoxy)ethoxy)benzoate (23c). Compound 22c (0.79 g, 1.81 mmol, 1 equiv) and K₂CO₃ (1.50 g, 10.86 mmol, 6 equiv) were stirred in dry DMF (30 mL). Compound 15 (1.50 g, 3.80 mol, 2.1 equiv) was added and the mixture was stirred at 70° C. under N₂ atmosphere for 12 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with hexane/EtOAc=1/3 as the eluent to give the title compound as a yellow oil (1.31 g, 82%).

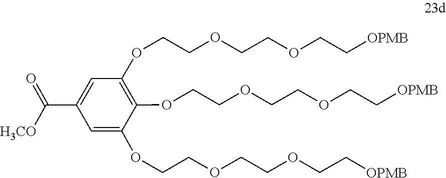

Methyl 3-(2-(2-(2-(benzyloxy)ethoxy)ethoxy)ethoxy)-4,5-bis(2-(2-(2-((4-methoxybenzyl)oxy)ethoxy) ethoxy)ethoxy)benzoate (23d). Compound 22a (1.80 g, 4.43 mmol, 1 equiv) and K₂CO₃ (3.67 g, 26.58 mmol, 6 equiv) were stirred in dry DMF (50 mL). Compound 17 (3.95 g, 9.30 mol, 2.1 equiv) was added and the mixture was stirred at 70° C. under N₂ atmosphere for 12 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with EtOAc as the eluent to give the title compound as a brown-yellow oil (3.65 g, 91%).

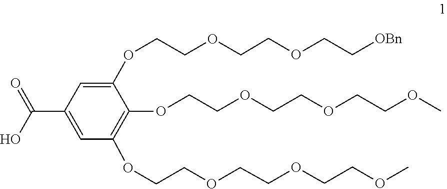 [SMILES: COCCOCCOCCOc1cc(C(=O)O)cc(OCCOCCOCCOCc2ccccc2)c1OCCOCCOCCOC]

3-(2-(2-(2-(Benzyloxy)ethoxy)ethoxy)ethoxy)-4,5-bis(2-(2-(2-methoxyethoxy)ethoxy)ethoxy) benzoic acid (1). Compound 23a (1.10 g, 1.57 mmol, 1 equiv) was dissolved in ethanol (EtOH, 15 mL). KOH (0.62 g, 10.99 mmol, 7 equiv) was added and the mixture was stirred at reflux for 2 h. The mixture was cooled to 23° C. and acidified with dilute HCl (1 M) to pH≈1. The solution was extracted by DCM (20 mL×3) and the organic phase was dried over anhydrous MgSO₄. Filtration and evaporation of the solvent yielded the title compound as a yellow oil (1.07 g, 100%).

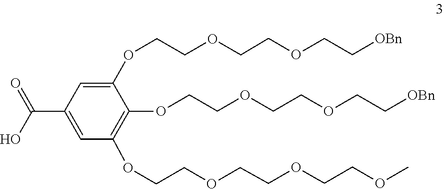 [SMILES: COCCOCCOCCOc1cc(C(=O)O)cc(OCCOCCOCCOCc2ccccc2)c1OCCOCCOCCOCc1ccccc1]

3,4-Bis(2-(2-(2-(benzyloxy)ethoxy)ethoxy)ethoxy)-5-(2-(2-(2-methoxyethoxy)ethoxy)ethoxy) benzoic acid (3). Compound 23b (7.88 g, 10.17 mmol, 1 equiv) was dissolved in ethanol (50 mL). KOH (3.99 g, 71.19 mmol, 7 equiv) was added and the mixture was stirred at reflux for 2 h. The mixture was cooled to 23° C. and acidified with dilute HCl (1 M) to pH≈1. The solution was extracted by DCM (40 mL×3) and the organic phase was dried over anhydrous MgSO₄. Filtration and evaporation of the solvent yielded the title compound as a yellow oil (7.73 g, 100%).

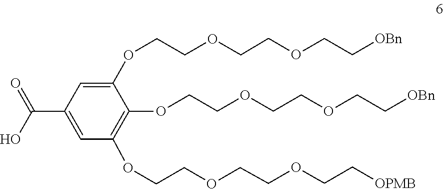

3,4-Bis(2-(2-(2-(benzyloxy)ethoxy)ethoxy)ethoxy)-5-(2-(2-(2-((4-methoxybenzyl)oxy)ethoxy)ethoxy) ethoxy)benzoic acid (6). Compound 23c (0.98 g, 1.11 mmol, 1 equiv) was dissolved in ethanol (15 mL). KOH (0.44 g, 7.77 mmol, 7 equiv) was added and the mixture was stirred at reflux for 2 h. The mixture was cooled to 23° C. and acidified with dilute HCl (1 M) to pH<2. The solution was extracted by DCM (30 mL×3) and the organic phase was dried over anhydrous MgSO₄. Filtration and evaporation of the solvent yielded the title compound as a brown-yellow oil (0.96 g, 100%).

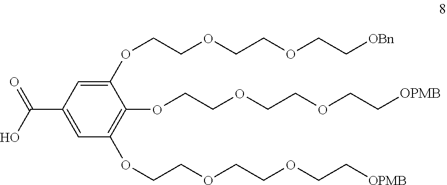

3-(2-(2-(2-(Benzyloxy)ethoxy)ethoxy)ethoxy)-4,5-bis(2-(2-(2-((4-methoxybenzyl)oxy)ethoxy)ethoxy) ethoxy)benzoic acid (8). Compound 23d (2.80 g, 3.07 mmol, 1 equiv) was dissolved in ethanol (30 mL). KOH (1.20 g, 21.49 mmol, 7 equiv) was added and the mixture was stirred at reflux for 2 h. The mixture was cooled to 23° C. and acidified with dilute HCl (1 M) to pH<2. The solution was extracted by DCM (30 mL×3) and the organic phase was dried over anhydrous MgSO₄. Filtration and evaporation of the solvent yielded the title compound as a brown-yellow oil (2.73 g, 100%).

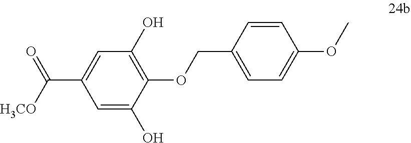 [SMILES: COC(=O)c1cc(O)c(OCc2ccc(OC)cc2)c(O)c1]

Methyl 3,5-dihydroxy-4-((4-methoxybenzyl)oxy)benzoate (24b). Methyl 3,4,5-trihydroxybenzoate (19, 3.25 g, 17.66 mmol, 1 equiv), 4-methoxybenzyl chloride (2.77 g, 17.66 mmol, 1 equiv), KHCO₃ (2.53 g, 35.32 mmol, 3 equiv) and KI (18 mg, 0.11 mmol, 0.006 equiv) were stirred in dry DMF (80 mL) at 60° C. under N₂ atmosphere for 24 h. The reaction mixture was cooled to 23° C. and DMF was removed under reduced pressure. Then water (50 mL) was added, and the mixture was extracted by DCM (50 mL×3). The organic phase was dried over anhydrous MgSO₄ and filtered. The filtrate was concentrated and purified by column chromatography (SiO₂) with hexane/EtOAc=2/1 as the eluent to give the title compound as a white solid (2.30 g, 76%).

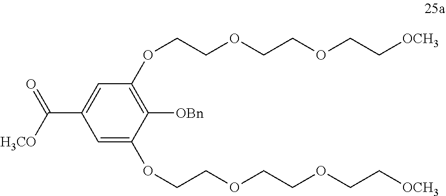 [SMILES: COCCOCCOCCOc1cc(C(=O)OC)cc(OCCOCCOCCOC)c1OCc1ccccc1]

Methyl 4-(benzyloxy)-3,5-bis(2-(2-(2-methoxyethoxy)ethoxy)ethoxy)benzoate (25a). Methyl 4-(benzyloxy)-3,5-dihydroxybenzoate (24a, 4.00 g, 14.59 mmol, 1 equiv) and K₂CO₃ (12.08 g, 87.41 mmol, 6 equiv) were stirred in dry DMF (70 mL). Compound 12 (10.64 g, 33.42 mol, 2.3 equiv) was added and the mixture was stirred at 70° C. under N₂ atmosphere for 12 h. The reaction mixture was cooled to 23° C. and DMF was removed under reduced pressure. Then water (50 mL) was added, and the mixture was extracted by DCM (30 mL×3). The organic phase was dried over anhydrous MgSO₄ and filtered. The filtrate was concentrated and purified by column chromatography (SiO₂) with EtOAc as the eluent to give the title compound as a light-yellow oil (7.27 g, 88%).

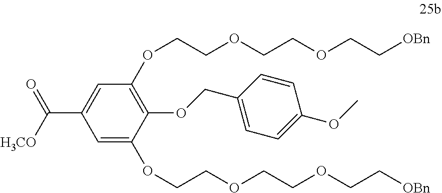 [SMILES: COC(=O)c1cc(OCCOCCOCCOCc2ccccc2)c(OCc2ccc(OC)cc2)c(OCCOCCOCCOCc2ccccc2)c1]

Methyl 3,5-bis(2-(2-(2-(benzyloxy)ethoxy)ethoxy)ethoxy)-4-((4-methoxybenzyl)oxy)benzoate (25b). Compound 24b (4.00 g, 13.14 mmol, 1 equiv) and K₂CO₃ (10.90 g, 78.86 mmol, 6 equiv) were stirred in dry DMF (60 mL). Compound 15 (11.90 g, 30.17 mol, 2.3 equiv) was added and the mixture was stirred at 70° C. under N₂ atmosphere for 12 h. The reaction mixture was cooled to 23° C. and DMF was removed under reduced pressure. Then water (50 mL) was added and the mixture was extracted by DCM (50 mL×3). The organic phase was dried over anhydrous MgSO₄ and filtered. The filtrate was concentrated and purified by column chromatography (SiO₂) with hexane/EtOAc=1/2 as the eluent to give the title compound as a light-yellow oil (7.70 g, 78%).

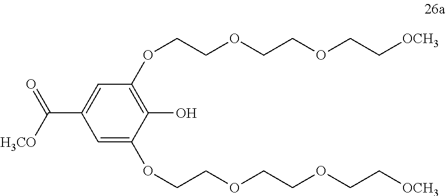 [SMILES: COCCOCCOCCOc1cc(C(=O)OC)cc(OCCOCCOCCOC)c1O]

Methyl 4-hydroxy-3,5-bis(2-(2-(2-methoxyethoxy)ethoxy)ethoxy)benzoate (26a). Compound 25a (7.27 g, 12.83 mmol) was dissolved in 1:2 MeOH:DCM (60 mL). Then Pd/C (0.22 g, 3 wt %) was added and the flask was evacuated and filled with hydrogen for three times. The mixture was stirred at 23° C. for 12 h under hydrogen atmosphere. The reaction mixture was filtered through Celite and the filter cake was washed with DCM. Evaporation of the solvent yielded the title compound as a light-yellow oil (6.11 g, 100%).

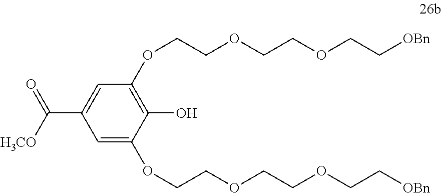 [SMILES: COC(=O)c1cc(OCCOCCOCCOCc2ccccc2)c(O)c(OCCOCCOCCOCc2ccccc2)c1]

Methyl 3,5-bis(2-(2-(2-(benzyloxy)ethoxy)ethoxy)ethoxy)-4-hydroxybenzoate (26b). 20 Compound 25b (3.00 g, 4.01 mmol) was dissolved in 20 mL DCM. Then trifluoroacetic acid (TFA, 1.83 g, 16.04 mmol) was added. The mixture was stirred at 23° C. for 2 h. The reaction was quenched by adding 30 mL saturated NaHCO₃solution and the mixture was extracted by DCM (30 mL×3). The combined organic phase was dried over anhydrous MgSO₄. Filtration and evaporation of the solvent yielded the title compound as a light-yellow oil (2.52 g, 100%).

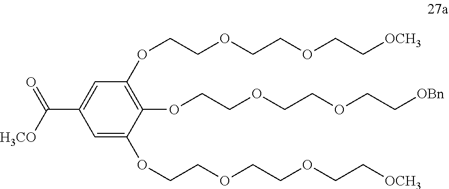 [SMILES: COCCOCCOCCOc1cc(C(=O)OC)cc(OCCOCCOCCOC)c1OCCOCCOCCOCc1ccccc1]

Methyl 4-(2-(2-(2-(benzyloxy)ethoxy)ethoxy)ethoxy)-3,5-bis(2-(2-(2-methoxyethoxy)ethoxy)ethoxy) benzoate (27a). Compound 26a (1.06 g, 2.22 mmol, 1 equiv) and K₂CO₃ (0.92 g, 6.66 mmol, 3 equiv) were stirred in dry DMF (30 mL). Compound 15 (1.00 g, 2.53 mmol, 1.2 equiv) was added and the mixture was stirred at 70° C. under N₂ atmosphere for 12 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with EtOAc/MeOH=30/1 as the eluent to give the title compound as a light-yellow oil (1.46 g, 94%).

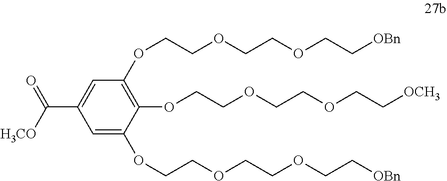 [SMILES: COCCOCCOCCOc1c(OCCOCCOCCOCc2ccccc2)cc(C(=O)OC)cc1OCCOCCOCCOCc1ccccc1]

Methyl 3,5-bis(2-(2-(2-(benzyloxy)ethoxy)ethoxy)ethoxy)-4-(2-(2-(2-methoxyethoxy)ethoxy)ethoxy) benzoate (27b). Compound 26b (1.70 g, 2.71 mmol, 1 equiv) and K₂CO₃ (1.12 g, 8.13 mmol, 3 equiv) were stirred in dry DMF (70 mL). Compound 12 (1.00 g, 3.25 mol, 1.2 equiv) was added and the mixture was stirred at 70° C. under N₂ atmosphere for 12 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with EtOAc/MeOH=50/1 as the eluent to give the title compound as a light-yellow oil (1.85 g, 89%).

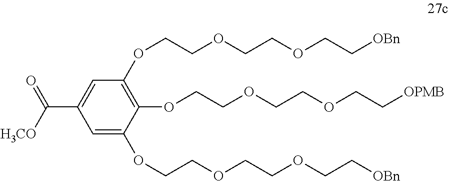

Methyl 3,5-bis(2-(2-(2-(benzyloxy)ethoxy)ethoxy)ethoxy)-4-(2-(2-(2-((4-methoxybenzyl)oxy)ethoxy) ethoxy)ethoxy)benzoate (27c). Compound 26b (1.00 g, 1.59 mmol, 1 equiv) and K₂CO₃ (0.66 g, 4.78 mmol, 3 equiv) were stirred in dry DMF (20 mL). Compound 17 (0.75 g, 1.75 mol, 1.1 equiv) was added and the mixture was stirred at 70° C. under N₂ atmosphere for 12 h. The reaction mixture was cooled to 23° C. and DMF was removed under reduced pressure. Then water (20 mL) was added, and the mixture was extracted by DCM (20 mL×3). The organic phase was dried over anhydrous MgSO₄ and filtered. The filtrate was concentrated and purified by column chromatography (SiO₂) with hexane/EtOAc=1/3 as the eluent to give the title compound as a yellow oil (1.21 g, 86%).

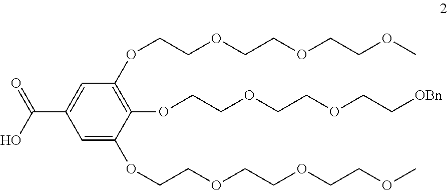 [SMILES: COCCOCCOCCOc1cc(C(=O)O)cc(OCCOCCOCCOC)c1OCCOCCOCCOCc1ccccc1]

4-(2-(2-(2-(Benzyloxy)ethoxy)ethoxy)ethoxy)-3,5-bis(2-(2-(2-methoxyethoxy)ethoxy)ethoxy) benzoic acid (2). Compound 27a (1.46 g, 2.09 mmol, 1 equiv) was dissolved in ethanol (15 mL). KOH (0.82 g, 14.62 mmol, 7 equiv) was added and the mixture was stirred at reflux for 2 h. The mixture was cooled to 23° C. and acidified with dilute HCl (1 M) to pH≈1. The solution was extracted by DCM (20 mL×3) and the organic phase was dried over anhydrous MgSO₄. Filtration and evaporation of the solvent yielded the title compound as a light-yellow oil (1.42 g, 100%).

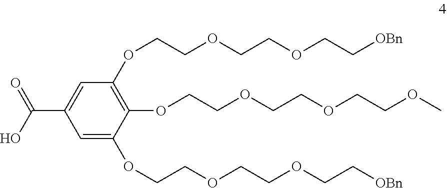 [SMILES: COCCOCCOCCOc1c(OCCOCCOCCOCc2ccccc2)cc(C(=O)O)cc1OCCOCCOCCOCc1ccccc1]

3,4-Bis(2-(2-(2-(benzyloxy)ethoxy)ethoxy)ethoxy)-5-(2-(2-(2-methoxyethoxy)ethoxy)ethoxy) benzoic acid (4). Compound 27b (1.80 g, 2.32 mmol, 1 equiv) was dissolved in ethanol (20 mL). KOH (0.90 g, 16.30 mmol, 7 equiv) was added and the mixture was stirred at reflux for 2 h. The mixture was cooled to 23° C. and acidified with dilute HCl (1 M) to pH≈1. The solution was extracted by DCM (20 mL×3) and the organic phase was dried over anhydrous MgSO₄. Filtration and evaporation of the solvent yielded the title compound as a yellow oil (1.76 g, 100%).

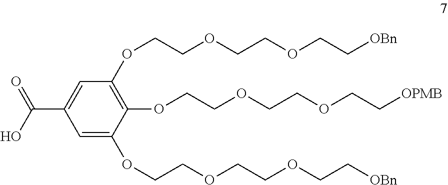

3,5-Bis(2-(2-(2-(benzyloxy)ethoxy)ethoxy)ethoxy)-4-(2-(2-(2-((4-methoxybenzyl)oxy)ethoxy)ethoxy) ethoxy)benzoic acid (7). Compound 27c (1.00 g, 1.14 mmol, 1 equiv) was dissolved in ethanol (15 mL). KOH (0.45 g, 7.98 mmol, 7 equiv) was added and the mixture was stirred at reflux for 2 h. The mixture was cooled to 23° C. and acidified with dilute HCl (1 M) to pH<2. The solution was extracted by DCM (30 mL×3) and the organic phase was dried over anhydrous MgSO₄. Filtration and evaporation of the solvent yielded the title compound as a brown-yellow oil (0.99 g, 100%).

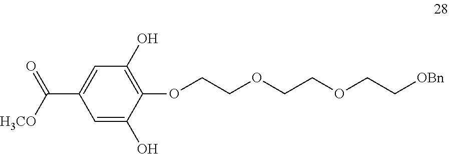 [SMILES: COC(=O)c1cc(O)c(OCCOCCOCCOCc2ccccc2)c(O)c1]

Methyl 4-(2-(2-(2-(benzyloxy)ethoxy)ethoxy)ethoxy)-3,5-dihydroxybenzoate (28). Compound 19 (2.73 g, 14.84 mmol, 1 equiv), compound 15 (5.85 g, 14.84 mmol, 1 equiv), KHCO₃ (2.97 g, 29.68 mmol, 2 equiv) and KI (15 mg, 0.09 mmol, 0.006 equiv) were stirred in dry DMF (50 mL) at 60° C. under N₂ atmosphere for 24 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with hexane/EtOAc=1/1 as the eluent to give the title compound as a yellow oil (5.12 g, 85%).

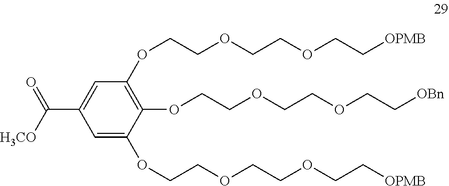

Methyl 4-(2-(2-(2-(benzyloxy)ethoxy)ethoxy)ethoxy)-3,5-bis(2-(2-(2-((4-methoxybenzyl)oxy)ethoxy) ethoxy)ethoxy)benzoate (29). Compound 28 (1.80 g, 4.43 mmol, 1 equiv) and K₂CO₃ (3.67 g, 26.58 mmol, 6 equiv) were stirred in dry DMF (50 mL). Compound 17 (3.95 g, 9.30 mol, 2.1 equiv) was added and the mixture was stirred at 70° C. under N₂ atmosphere for 12 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with EtOAc as the eluent to give the title compound as a yellow oil (3.42 g, 85%).

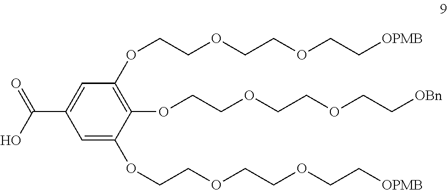

4-(2-(2-(2-(Benzyloxy)ethoxy)ethoxy)ethoxy)-3,5-bis(2-(2-(2-((4-methoxybenzyl)oxy)ethoxy)ethoxy) ethoxy)benzoic acid (9). Compound 29 (3.40 g, 3.73 mmol, 1 equiv) was dissolved in ethanol (30 mL). KOH (1.46 g, 26.11 mmol, 7 equiv) was added and the mixture was stirred at reflux for 2 h. The mixture was cooled to 23° C. and acidified with dilute HCl (1 M) to pH<2. The solution was extracted by DCM (30 mL×3) and the organic phase was dried over anhydrous MgSO₄. Filtration and evaporation of the solvent yielded the title compound as a yellow oil (3.35 g, 100%).

### Synthesis of Hydrophobic Benzyl Amines

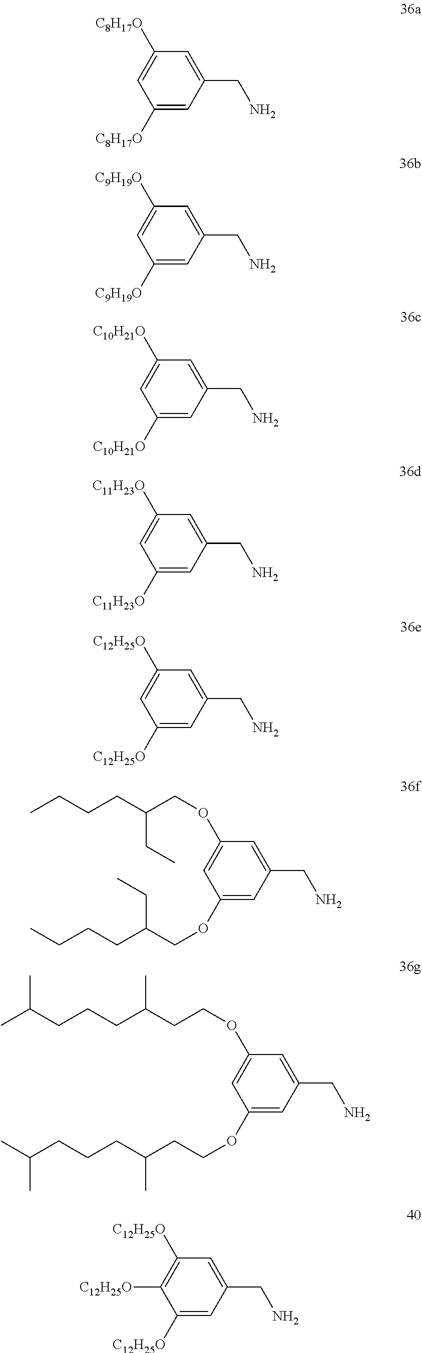

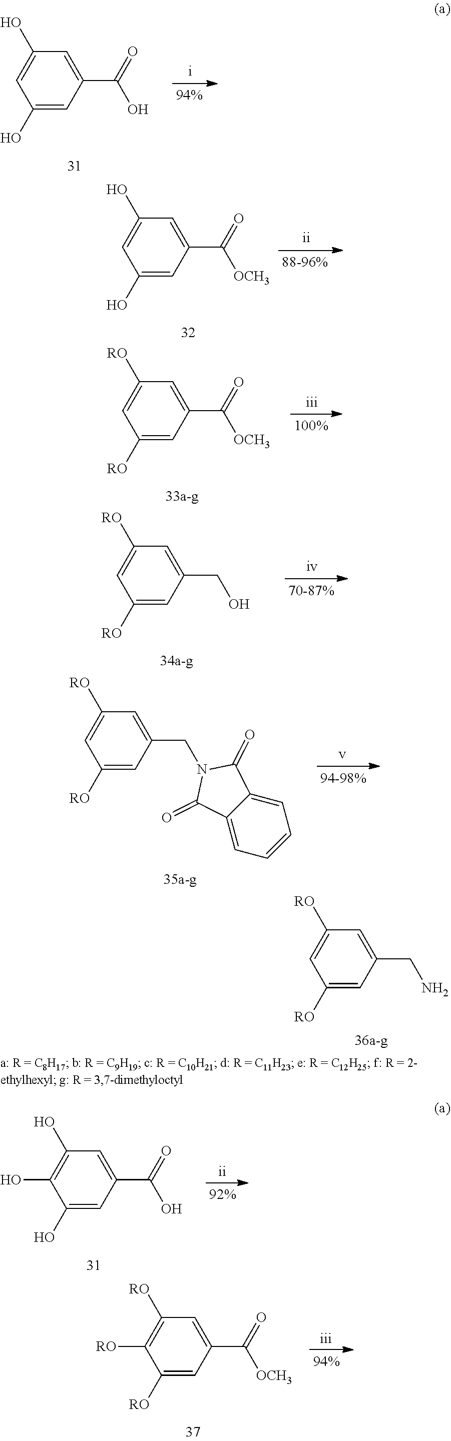

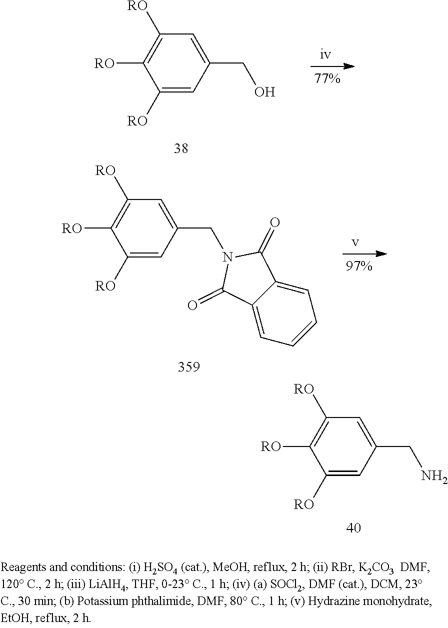

General Synthetic Procedure for Compounds 33a-g and 37: Compounds 33a-g and 37 were synthesized according to a literature procedure (Buzzacchera, et al., Biomacromolecules 2019, 20, 712-727). Generally, compound 32/19 (1 equiv) and K₂CO₃ (3 equiv) were stirred in dry DMF. RBr (2.2 equiv for compounds 33a-g, 3.3 equiv for compound 37) was added, and the mixture was stirred at 120° C. under N₂ atmosphere for 2 h. The reaction mixture was cooled to 23° C. For compounds 33a-e and 37, the reaction mixture was poured into ice/water and the white precipitates were filtered. Then the precipitates were recrystallized from acetone to afford the title compound as a white solid. For compounds 33f, g, water was added to the reaction mixture and the mixture was extracted by DCM for three times. The organic phase was dried over anhydrous MgSO₄ and filtered. The filtrate was concentrated and purified by column chromatography (SiO₂) with hexane/EtOAc=20/1 as the eluent to give the title compound as a light-yellow oil.

General Synthetic Procedure for Compounds 34a-g and 38. Compounds 34a-g and 38 were synthesized according to a literature procedure (Li, et al., ACS Nano 2020, 14, 7398-7411). Generally, compound 33a-g and 38 (1 equiv) dissolved in dry THF was added dropwise to a slurry of LiAlH₄ (1.2 equiv) in dry THF at 0° C. under N₂ atmosphere. The mixture was stirred at 23° C. for 1 h. Afterwards, the reaction was quenched by the successive addition of water, 15% NaOH aqueous solution and water. Then the mixture was filtered and dried over anhydrous MgSO₄. Filtration and evaporation of the solvent yielded the title compound as a white solid or light-yellow oil.

General Synthetic Procedure for Compounds 35a-g and 39. Compounds 35a-g and 39 were synthesized according to a literature procedure (Zhang, et al., ACS Nano 2014, 8, 1554-1565). The characterizations of 2-(3,5-bis(dodecyloxy)benzyl)isoindoline-1,3-dione (35e) and 2-(3,4,5-tris(dodecyloxy)benzyl)isoindoline-1,3-dione (39) were reported in the literature. A typical example is provided below.

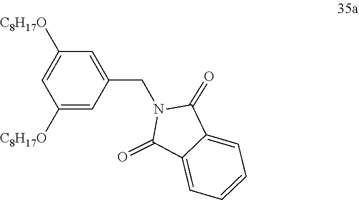

2-(3,5-Bis(octyloxy)benzyl)isoindoline-1,3-dione (35a). To a solution of 34a (3.82 g, 10.48 mmol, 1 equiv) in dry DCM (20 mL) was added 2 drops of DMF, followed by the dropwise addition of SOCl₂ (1.87 g, 15.72 mmol, 1.5 equiv). The reaction mixture was stirred at 23° C. for 30 min. Then DCM and excess SOCl₂ was removed under vacuum. The obtained intermediate product was dissolved in dry DMF (40 mL) and potassium phthalimide (2.15 g, 11.53 mmol, 1.1 equiv) was added. The mixture was stirred at 80° C. under N₂ atmosphere for 1 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with hexane/EtOAc=10/1 as the eluent to give the title compound as a yellow oil (4.64 g, 81%).

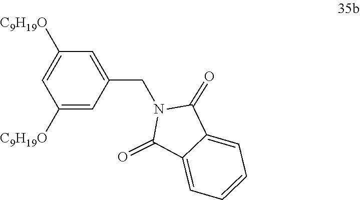

2-(3,5-Bis(nonyloxy)benzyl)isoindoline-1,3-dione (35b). From compound 34b (2.15 g, 5.47 mmol), SOCl₂ (0.60 g, 5.51 mmol) and potassium phthalimide (1.21 g, 6.56 mmol), compound 35b was obtained as a white solid (2.01 g, 70%).

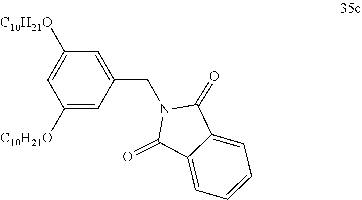

2-(3,5-Bis(decyloxy)benzyl)isoindoline-1,3-dione (35c). From compound 34c (2.94 g, 6.17 mmol), SOCl₂ (1.10 g, 9.26 mmol) and potassium phthalimide (1.26 g, 6.79 mmol), compound 35c was obtained as a red oil (2.84 g, 76%).

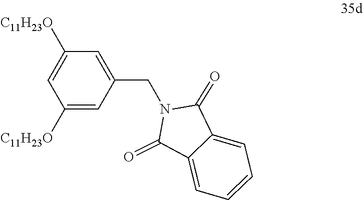

2-(3,5-Bis(undecyloxy)benzyl)isoindoline-1,3-dione (35d). From compound 34d (3.10 g, 7.90 mmol), SOCl₂ (1.13 g, 9.48 mmol) and potassium phthalimide (1.76 g, 9.48 mmol), compound 35d was obtained as a yellow solid 3.28 g, 72%).

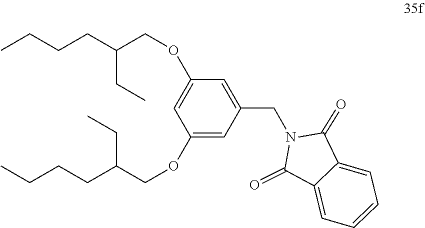 [SMILES: CCCCC(CC)COc1cc(CN2C(=O)c3ccccc3C2=O)cc(OCC(CC)CCCC)c1]

2-(3,5-Bis((2-ethylhexyl)oxy)benzyl)isoindoline-1,3-dione (35f). From compound 34f (3.20 g, 8.78 mmol), SOCl₂ (1.57 g, 13.17 mmol) and potassium phthalimide (1.80 g, 9.66 mmol), compound 35f was obtained as a yellow oil (3.25 g, 75%).

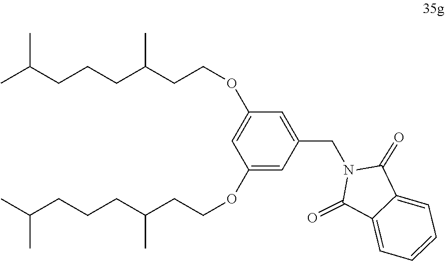 [SMILES: CC(C)CCCC(C)CCOc1cc(CN2C(=O)c3ccccc3C2=O)cc(OCCC(C)CCCC(C)C)c1]

2-(3,5-Bis((3,7-dimethyloctyl)oxy)benzyl)isoindoline-1,3-dione (35g). From compound 34g (3.50 g, 8.33 mmol), SOCl₂ (1.19 g, 10.0 mmol) and potassium phthalimide (1.85 g, 10.0 mmol), compound 35g was obtained as a yellow oil (3.98 g, 87%).

General Synthetic Procedure for Compounds 36a-g and 40. Compounds 36a-g and 40 were synthesized according to a literature procedure (Zhang, et al., ACS Nano 2014, 8, 1554-1565). The characterizations of (3,5-bis(dodecyloxy)phenyl)methanamine (36e) and (3,4,5-tris(dodecyloxy)phenyl)methanamine (40) were reported in the literature. A typical example is provided below.

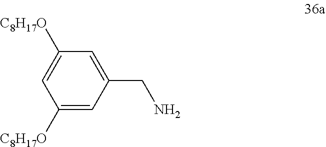

(3,5-Bis(octyloxy)phenyl)methanamine (36a). Compound 35a (1.52 g, 2.76 mmol, 1 equiv) and NH₂NH₂·H₂O (1.38 g, 27.6 mmol, 10 equiv) were dissolved in EtOH (40 mL). The mixture was heated at reflux for 2 h. The reaction mixture was cooled to 23° C. and EtOH was removed under reduced pressure. Then water (30 mL) was added, and the mixture was extracted by DCM (30 mL×3). The organic phase was dried over anhydrous MgSO₄ and filtered. Filtration and evaporation of the solvent yielded the title compound as a yellow oil (0.98 g, 98%).

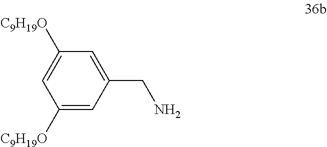

(3,5-Bis(nonyloxy)phenyl)methanamine (36b). From compound 35b (2.01 g, 3.85 mmol) and NH₂NH₂·H₂O (1.92 g, 38.5 mmol), compound 36b was obtained as a yellow oil (1.42 g, 94%).

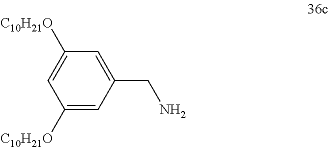

(3,5-Bis(decyloxy)phenyl)methanamine (36c). From compound 35c (1.87 g, 3.09 mmol) and NH₂NH₂·H₂O (1.55 g, 30.9 mmol), compound 36c was obtained as a brownish-red oil (1.26 g, 97%).

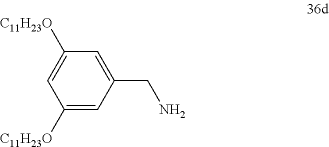

(3,5-Bis(undecyloxy)phenyl)methanamine (36d). From compound 35d (3.22 g, 5.57 mmol) and NH₂NH₂·H₂O (2.79 g, 55.7 mmol), compound 36d was obtained as a brownish-red oil (2.35 g, 94%).

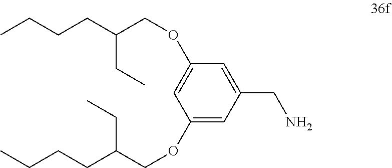 [SMILES: CCCCC(CC)COc1cc(CN)cc(OCC(CC)CCCC)c1]

(3,5-Bis((2-ethylhexyl)oxy)phenyl)methanamine (36f). From compound 35f (2.49 g, 5.04 mmol) and NH₂NH₂·H₂O (2.52 g, 50.4 mmol), compound 36f was obtained as a brownish-yellow oil (1.80 g, 98%).

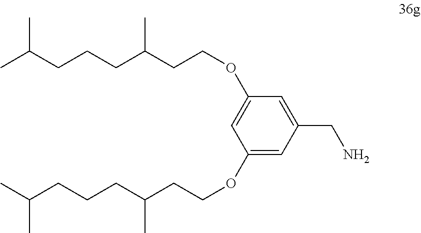 [SMILES: CC(C)CCCC(C)CCOc1cc(CN)cc(OCCC(C)CCCC(C)C)c1]

(3,5-Bis((3,7-dimethyloctyl)oxy)phenyl)methanamine (36g). From compound 35g (3.50 g, 6.4 mmol) and NH₂NH₂·H₂O (3.19 g, 64 mmol), compound 36g was obtained as a brownish-yellow oil (2.55 g, 95%).

### Synthesis of Hydrophobic Acids

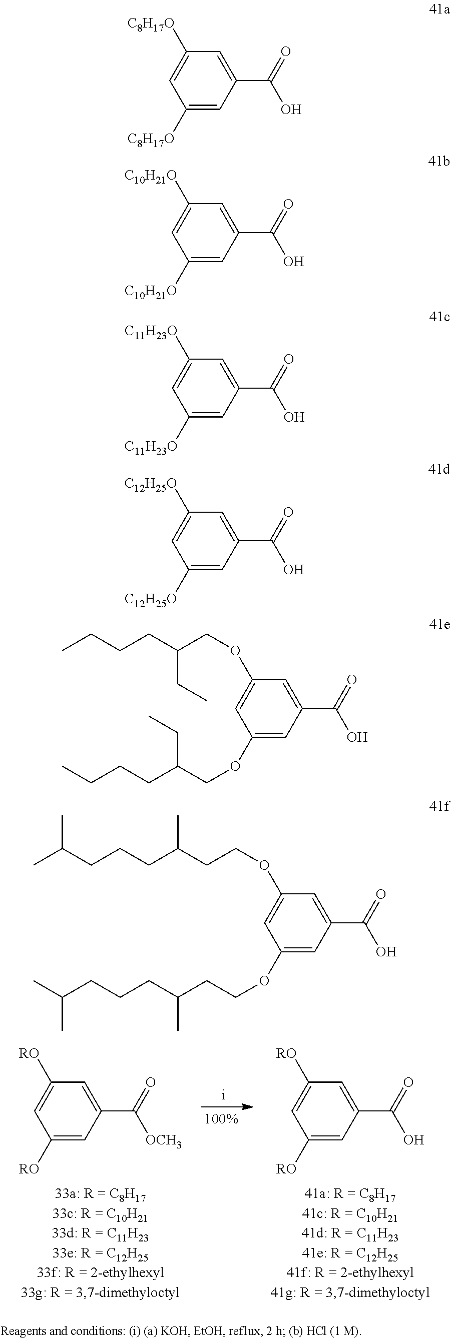

Compounds 41a-f were synthesized according to a literature procedure. The procedures were optimized to increase the yields of compounds 41a-f to around 20 g, respectively.

### Synthesis of Twin-Twin Hydrophobic Building Blocks

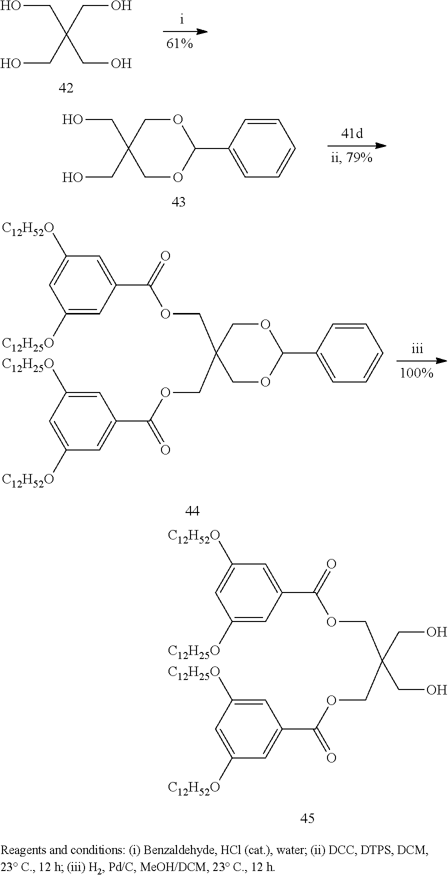

Compounds 43-45 were synthesized according to a literature procedure. The procedures were optimized to increase the yields of compound 45 to 30 g.

### 3.2 Synthesis of Single-Single IAJDs of Library 1

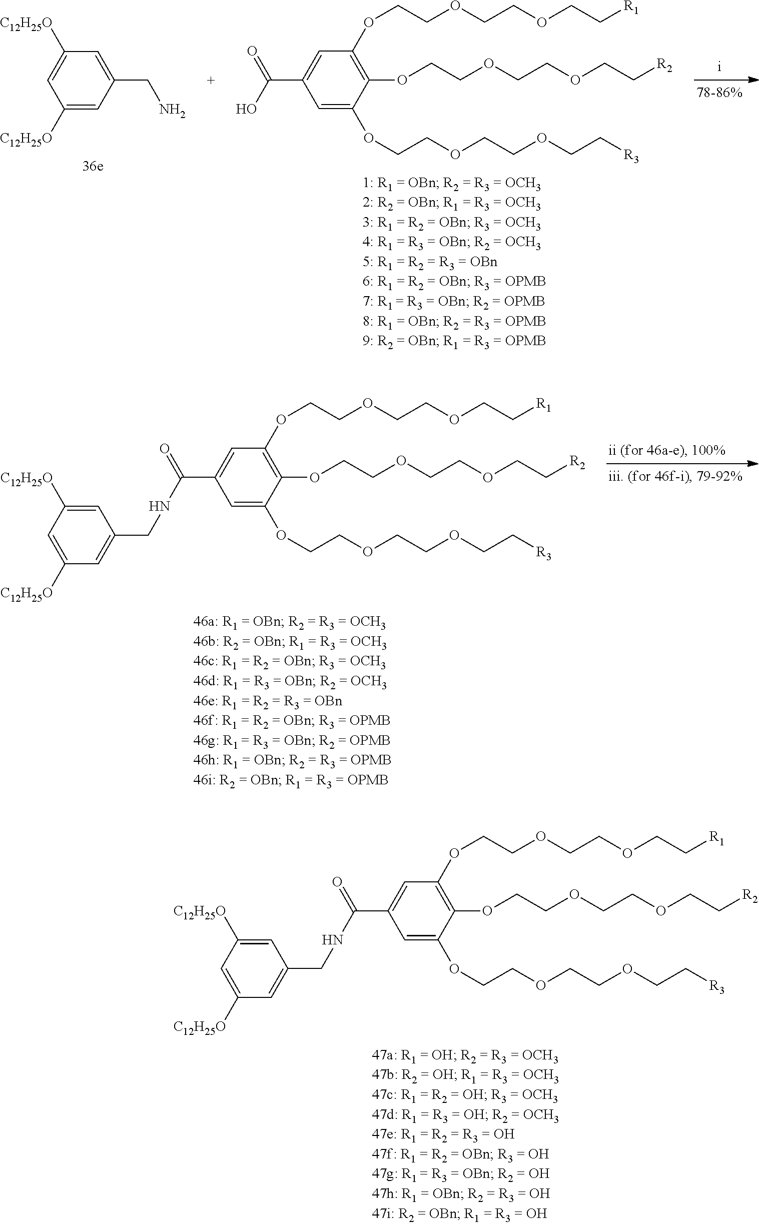

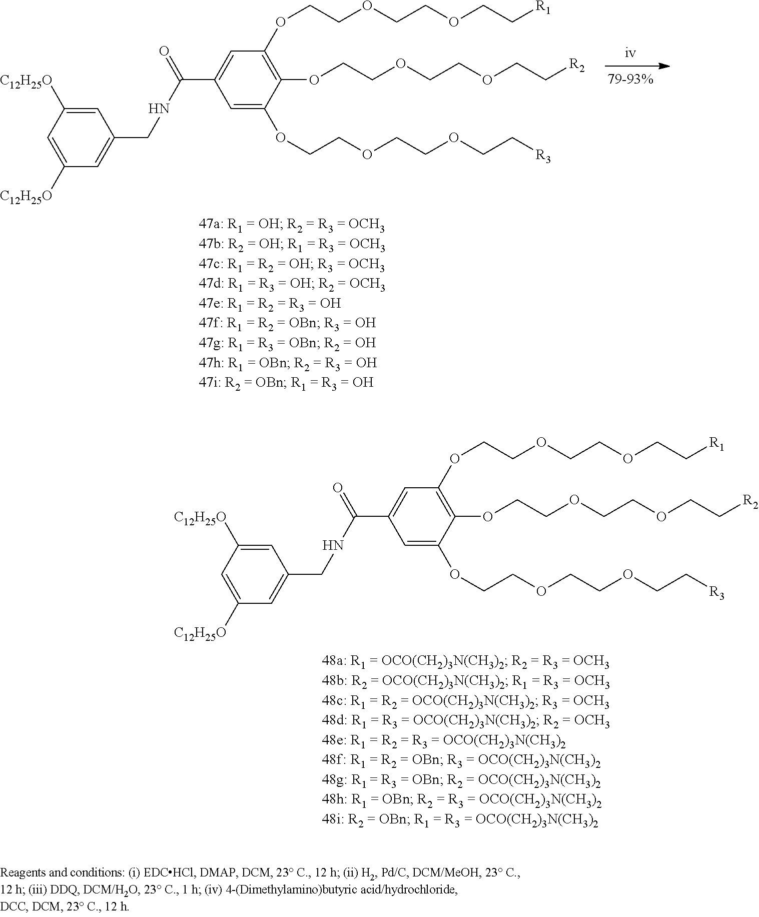

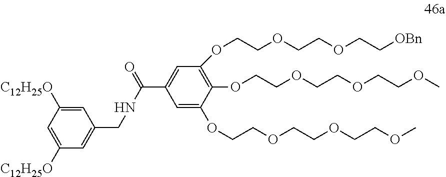

Compound 46a. Compound 1 (0.36 g, 0.53 mmol, 1 equiv), compound 36e (0.27 g, 0.57 mmol, 1.1 equiv), EDC·HCl (0.11 g, 0.57 mmol, 1.1 equiv) and DMAP (19 mg, 0.16 mmol, 0.3 equiv) were dissolved in dry DCM (6 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/MeOH=30/1 as the eluent to give the title compound as a light-yellow oil (0.51 g, 85%).

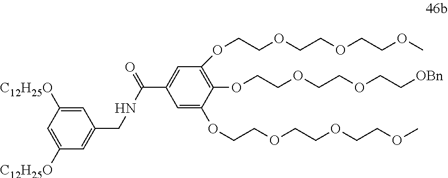

Compound 46b. Compound 2 (0.36 g, 0.53 mmol, 1 equiv), compound 36e (0.27 g, 0.57 mmol, 1.1 equiv), EDC·HCl (0.11 g, 0.57 mmol, 1.1 equiv) and DMAP (19 mg, 0.16 mmol, 0.3 equiv) were dissolved in dry DCM (6 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/MeOH=30/1 as the eluent to give the title compound as a light-yellow oil (0.49 g, 82%).

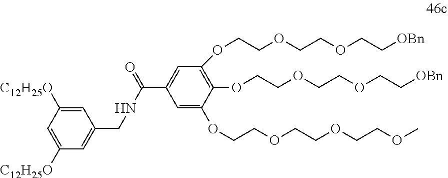

Compound 46c. Compound 3 (1.15 g, 1.51 mmol, 1 equiv), compound 36e (0.80 g, 1.68 mmol, 1.1 equiv), EDC·HCl (0.32 g, 1.68 mmol, 1.1 equiv) and DMAP (55 mg, 0.45 mmol, 0.3 equiv) were dissolved in dry DCM (10 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (1.55 g, 84%).

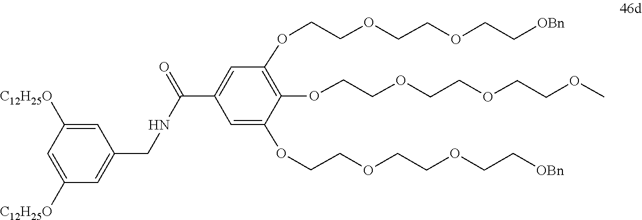

Compound 46d. Compound 4 (1.15 g, 1.51 mmol, 1 equiv), compound 36e (0.80 g, 1.68 mmol, 1.1 equiv), EDC·HCl (0.32 g, 1.68 mmol, 1.1 equiv) and DMAP (55 mg, 0.45 mmol, 0.3 equiv) were dissolved in dry DCM (10 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (1.44 g, 78%).

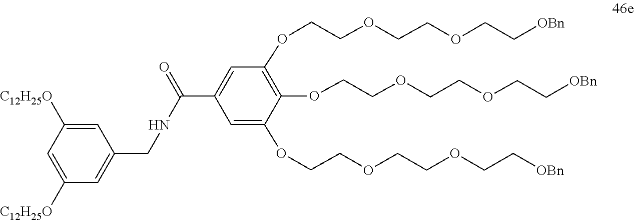

Compound 46e. Compound 5 (0.37 g, 0.44 mmol, 1 equiv), compound 36e (0.23 g, 0.48 mmol, 1.1 equiv), EDC·HCl (92 mg, 0.48 mmol, 1.1 equiv) and DMAP (27 mg, 0.22 mmol, 0.5 equiv) were dissolved in dry DCM (8 mL). The reaction mixture was stirred at 23° C. for 12 h. Brine (20 mL) was added, and the mixture was extracted by DCM (20 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (0.48 g, 84%).

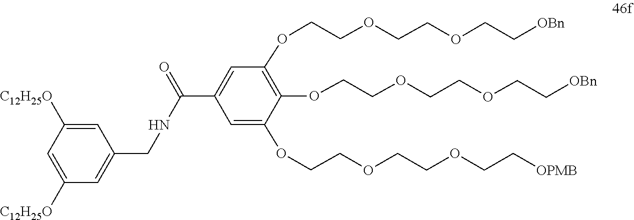

Compound 46f. Compound 6 (0.45 g, 0.52 mmol, 1 equiv), compound 36e (0.27 g, 0.57 mmol, 1.1 equiv), EDC·HCl (0.11 g, 0.57 mmol, 1.1 equiv) and DMAP (19 mg, 0.16 mmol, 0.3 equiv) were dissolved in dry DCM (6 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (0.59 g, 86%).

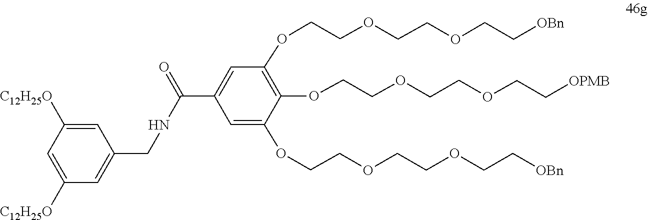

Compound 46g. Compound 7 (0.45 g, 0.52 mmol, 1 equiv), compound 36e (0.27 g, 0.57 mmol, 1.1 equiv), EDC·HCl (0.11 g, 0.57 mmol, 1.1 equiv) and DMAP (19 mg, 0.16 mmol, 0.3 equiv) were dissolved in dry DCM (6 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (0.54 g, 78%).

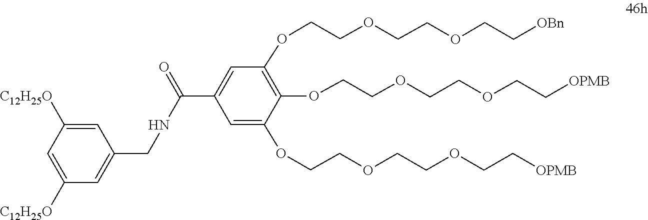

Compound 46h. Compound 8 (0.83 g, 0.92 mmol, 1 equiv), compound 36e (0.48 g, 1.01 mmol, 1.1 equiv), EDC·HCl (0.19 g, 1.01 mmol, 1.1 equiv) and DMAP (34 mg, 0.28 mmol, 0.3 equiv) were dissolved in dry DCM (8 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (1.06 g, 85%).

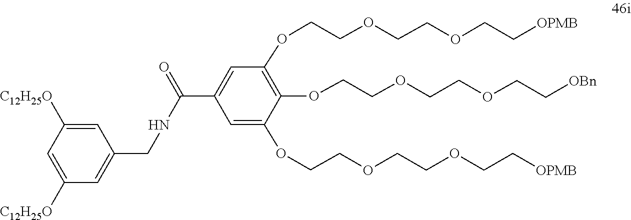

Compound 46i. Compound 9 (0.86 g, 0.96 mmol, 1 equiv), compound 36e (0.50 g, 1.05 mmol, 1.1 equiv), EDC·HCl (0.20 g, 1.05 mmol, 1.1 equiv) and DMAP (35 mg, 0.29 mmol, 0.3 equiv) were dissolved in dry DCM (8 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (1.07 g, 83%).

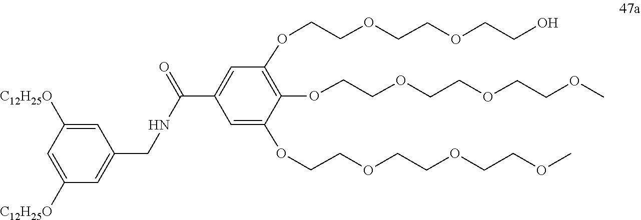

Compound 47a. Compound 46a (0.46 g, 0.40 mmol) was dissolved in 1:2 MeOH:DCM (15 mL). Then Pd/C (23.0 mg, 5 wt %) was added and the flask was evacuated and filled with hydrogen for three times. The mixture was stirred at 23° C. for 12 h under hydrogen atmosphere. The reaction mixture was filtered through Celite and the filter cake was washed with DCM. Evaporation of the solvent yielded the title compound as a colorless oil (0.42 g, 100%).

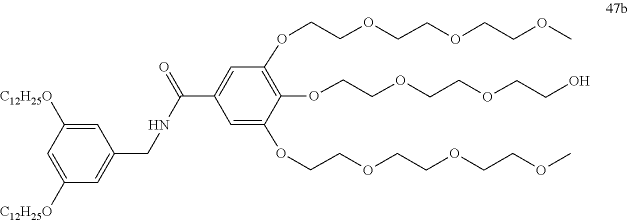

Compound 47b. Compound 47b was synthesized from compound 46b (0.45 g, 0.39 mmol) following a procedure similar to that used for the synthesis of compound 47a. The title compound was obtained as a light-yellow oil (0.41 g, 100%).

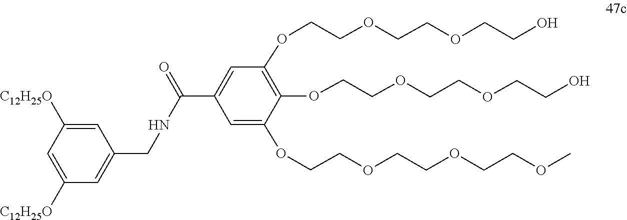

Compound 47c. Compound 47c was synthesized from compound 46c (1.55 g, 1.27 mmol) following a procedure similar to that used for the synthesis of compound 47a. The title compound was obtained as a colorless oil (1.31 g, 100%).

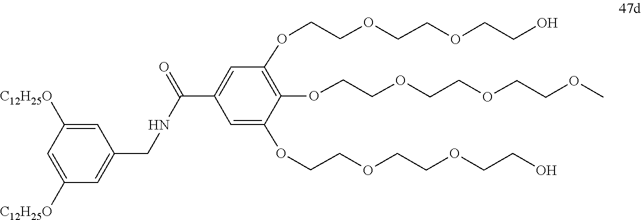

Compound 47d. Compound 47d was synthesized from compound 46d (1.40 g, 1.15 mmol) following a procedure similar to that used for the synthesis of compound 47a. The title compound was obtained as a colorless oil (1.19g, 100%)

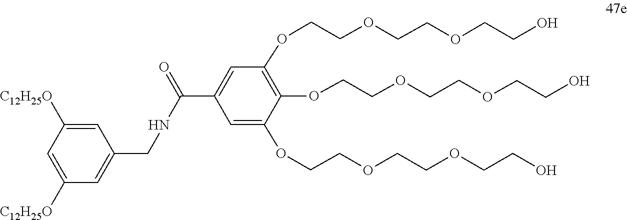

Compound 47e. Compound 47e was synthesized from compound 46e (0.40 g, 0.31 mmol) following a procedure similar to that used for the synthesis of compound 47a. The title compound was obtained as a light-yellow oil (0.31 g, 100%).

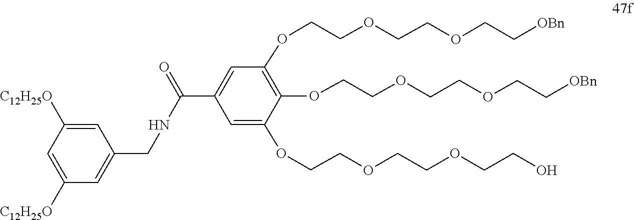

Compound 47f. Compound 46f (0.51 g, 0.38 mmol, 1 equiv) was dissolved in 5 mL DCM and 0.25 mL water (5%) was added. To this solution was added 2,3-dichloro-5,6-dicyano-1,4-benzoquinone (DDQ, 95 mg, 0.42 mmol, 1.1 equiv). The mixture was allowed to stir at 23° C. for 1 h. The precipitates were filtered out and DCM (20 mL) was added. The mixture was washed by NaHCO₃aqueous solution (saturated), NaHSO₃ aqueous solution (2%) and NaHCO₃aqueous solution (saturated) successively. The organic phase was dried over anhydrous MgSO₄ and filtered. The filtrate was concentrated and purified by column chromatography (SiO₂) with EtOAc/MeOH=20/1 as the eluent to give the title compound as a yellow oil (0.42 g, 91%).

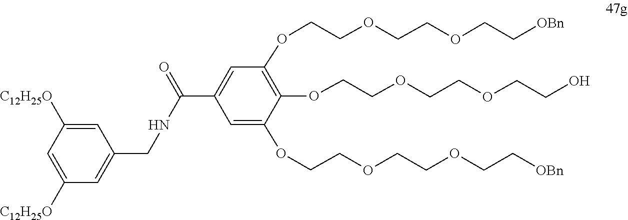

Compound 47g. Compound 47g was synthesized from compound 46g (0.44 g, 0.33 mmol) following a procedure similar to that used for the synthesis of compound 47f. The title compound was obtained as a yellow oil (0.39 g, 82%).

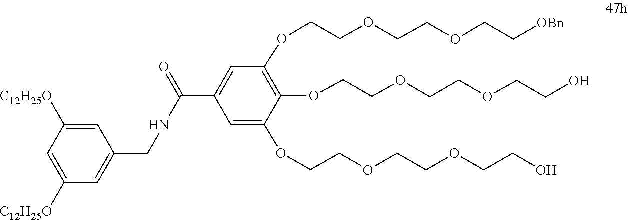

Compound 47h. Compound 47h was synthesized from compound 46h (0.74 g, 0.55 mmol) following a procedure similar to that used for the synthesis of compound 47f. The title compound was obtained as a light-yellow oil (0.56 g, 92%).

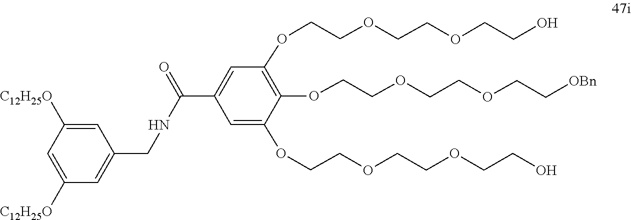

Compound 47i. Compound 47i was synthesized from compound 46i (0.80 g, 0.59 mmol) following a procedure similar to that used for the synthesis of compound 47f. The title compound was obtained as a light-yellow oil (0.51 g, 79%).

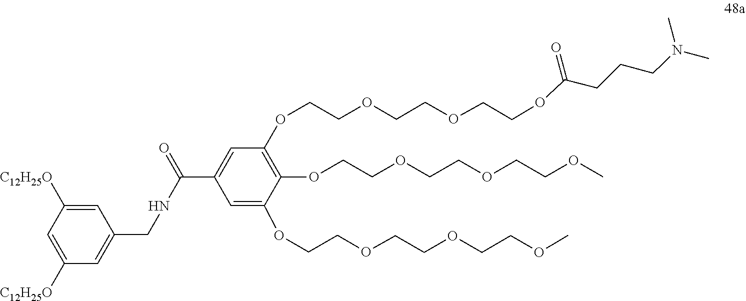

Compound 48a (3/1_{DMBA}¹). Compound 47a (0.40 g, 0.38 mmol, 1 equiv) and 4-(dimethylamino)butyric acid hydrochloride (70 mg, 0.42 mmol, 1.1 equiv) were dissolved in 8 mL dry DCM. N,N′-Dicyclohexylcarbodiimide (DCC, 0.17 g, 0.57 mmol, 1.5 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=8/1 as the eluent to give the title compound as a colorless oil (0.39 g, 89%).

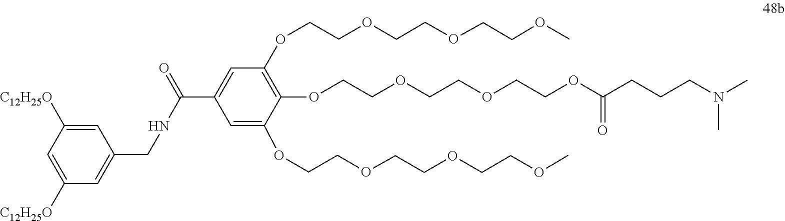

Compound 48b (3/1_{DMBA}²). Compound 47b (0.37 g, 0.35 mmol, 1 equiv) and 4-(dimethylamino)butyric acid hydrochloride (65 mg, 0.39 mmol, 1.1 equiv) were dissolved in 6 mL dry DCM. DCC (0.11 g, 0.53 mmol, 1.5 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=8/1 as the eluent to give the title compound as a light-yellow oil (0.37 g, 89%).

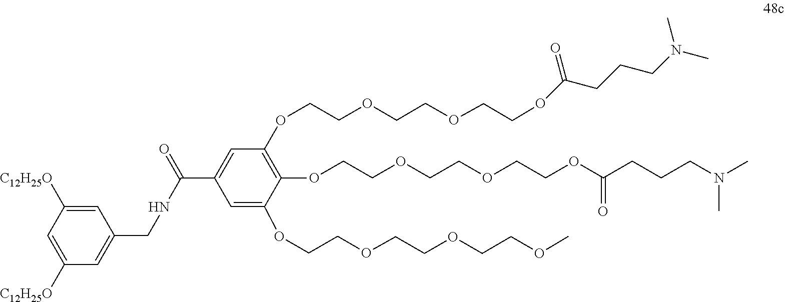

Compound 48c (3/2_{DMBA}^{1,2}). Compound 47c (0.40 g, 0.38 mmol, 1 equiv) and 4-(dimethylamino)butyric acid hydrochloride (0.15 g, 0.89 mmol, 2.3 equiv) were dissolved in 8 mL dry DCM. DCC (0.24 g, 1.16 mmol, 3 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=8/1, 6/1 with 0.1% triethylamine (TEA) as the eluent. Then the crude product was dissolved in sodium bicarbonate (NaHCO₃) solution (2%, 30 mL) and the mixture was extracted by DCM (30 mL×3). The organic phase was dried over anhydrous MgSO₄. Filtration and evaporation of the solvent yielded the title compound as a light-yellow oil (0.38 g, 79%).

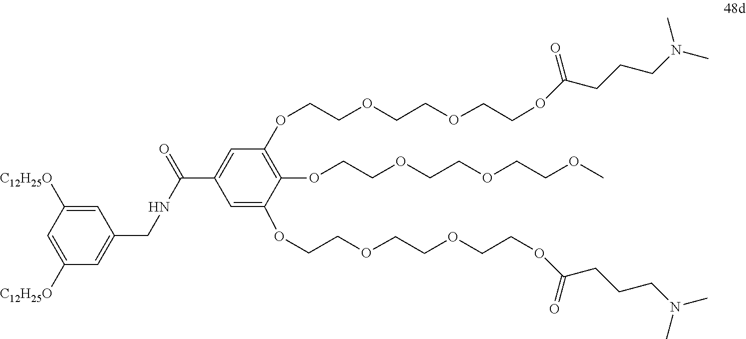

Compound 48d (3/2_{DMBA}^{1,3}). Compound 47d (0.26 g, 0.25 mmol, 1 equiv) and 4-(dimethylamino)butyric acid hydrochloride (97 mg, 0.58 mmol, 2.3 equiv) were dissolved in 6 mL dry DCM. DCC (0.16 g, 0.75 mmol, 3 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=8/1, 6/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (0.28 g, 88%).

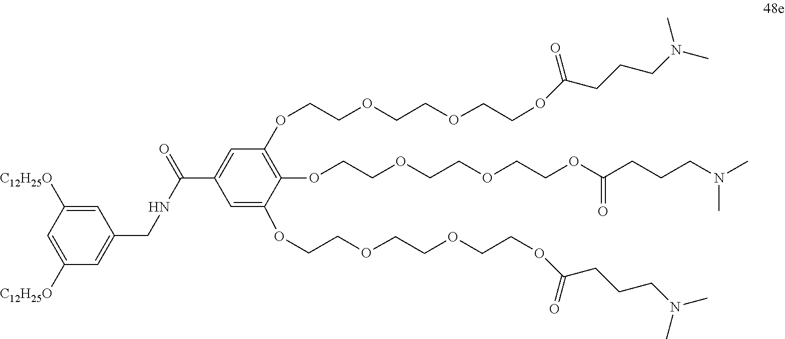

Compound 48e (3/3_{DMBA}^{1,2,3}). Compound 47e (0.27 g, 0.26 mmol, 1 equiv) and 4-(dimethylamino)butyric acid hydrochloride (0.15 g, 0.87 mmol, 3.3 equiv) were dissolved in 6 mL dry DCM. DCC (0.24 g, 1.17 mmol, 4.5 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=8/1, 6/1 and 4/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a colorless oil (0.31 g, 88%).

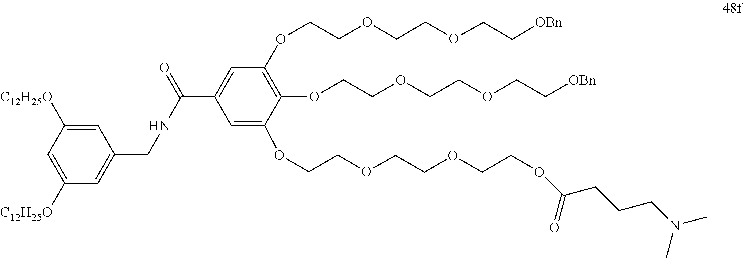

Compound 48f (3/1_{DMBA}¹_{Bn}^{2,3}). Compound 47f (0.30 g, 0.25 mmol, 1 equiv) and 4-(dimethylamino)butyric acid hydrochloride (50 mg, 0.30 mmol, 1.2 equiv) were dissolved in 6 mL dry DCM. DCC (0.11 g, 0.50 mmol, 2 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=7/1 as the eluent to give the title compound as a light-yellow oil (0.29 g, 88%).

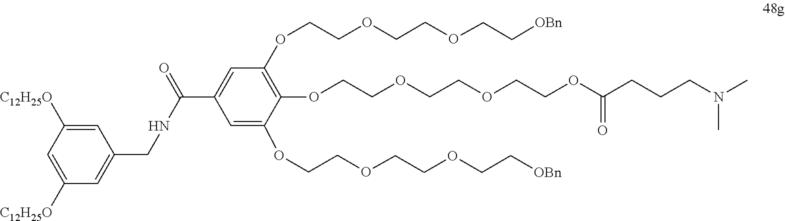

Compound 48g (3/1_{DMBA}²_{Bn}^{1,3}). Compound 47g (0.29 g, 0.24 mmol, 1 equiv) and 4-(dimethylamino)butyric acid hydrochloride (49 mg, 0.29 mmol, 1.2 equiv) were dissolved in 6 mL dry DCM. DCC (0.10 g, 0.48 mmol, 2 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=6/1 as the eluent to give the title compound as a light-yellow oil (0.28 g, 86%).

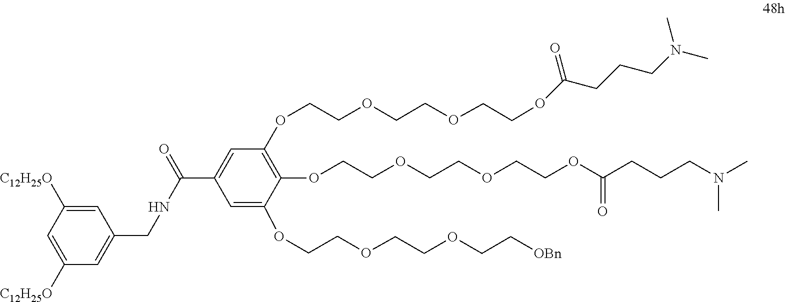

Compound 48h (3/2_{DMBA}^{1,2}_{Bn}³). Compound 47h (0.31 g, 0.28 mmol, 1 equiv) and 4-(dimethylamino)butyric acid hydrochloride (0.11 g, 0.66 mmol, 2.3 equiv) were dissolved in 6 mL 20 dry DCM. DCC (0.18 g, 0.84 mmol, 3 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=8/1, 6/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (0.35 g, 93%).

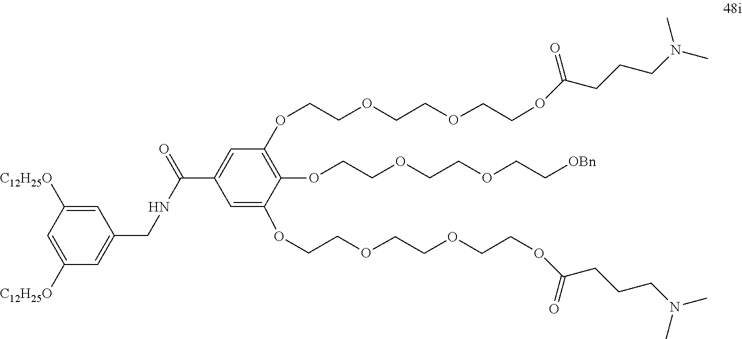

Compound 48i (3/2_{DMBA}^{1,3}_{Bn}²). Compound 47i (0.43 g, 0.39 mmol, 1 equiv) and 4-(dimethylamino)butyric acid hydrochloride (0.14 g, 0.85 mmol, 2.3 equiv) were dissolved in 6 mL dry DCM. DCC (0.24 g, 1.16 mmol, 3 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=8/1, 6/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (0.42 g, 82%).

### 3.3 Synthesis of Single-Single IAJDs in Library 2

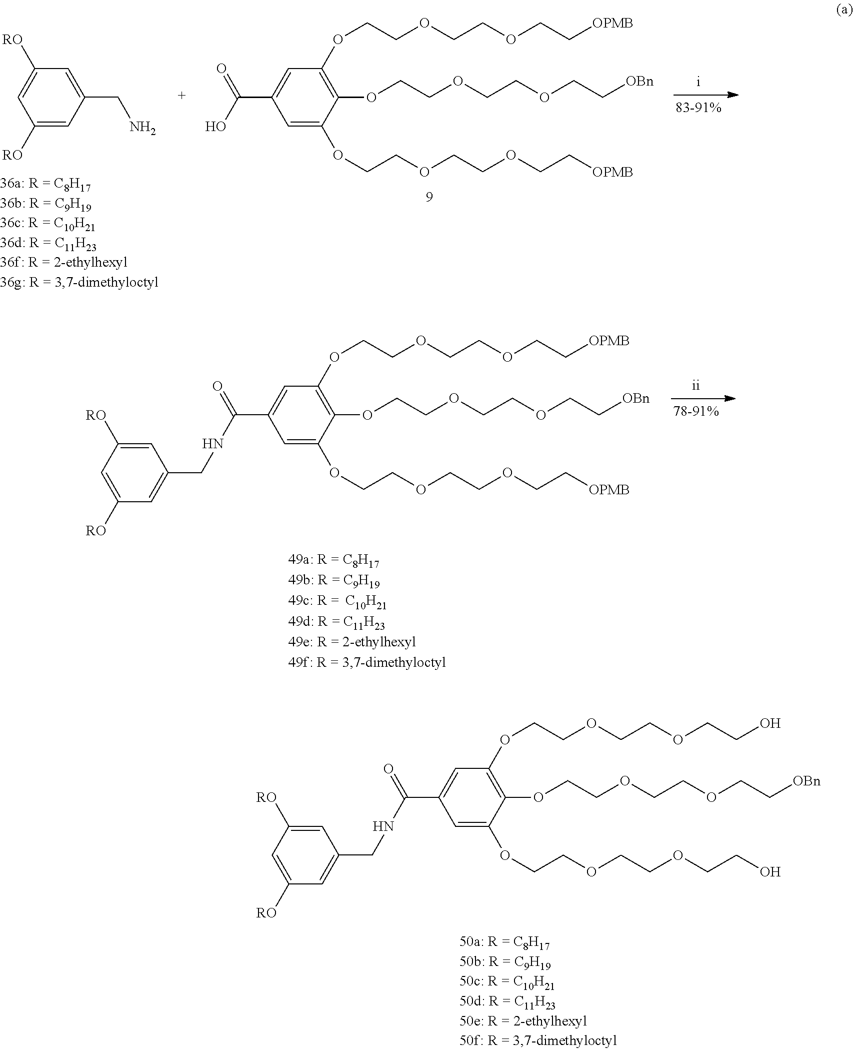

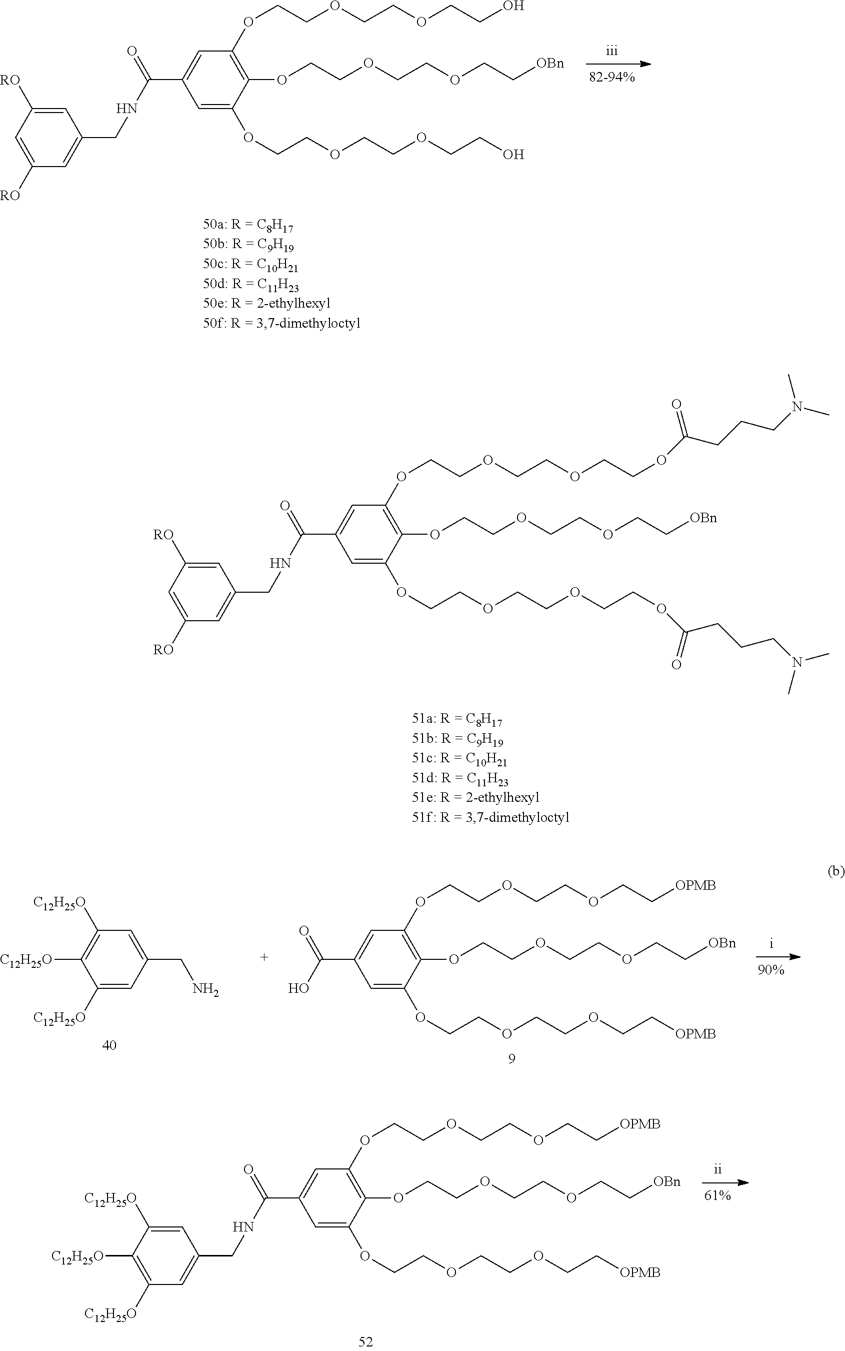

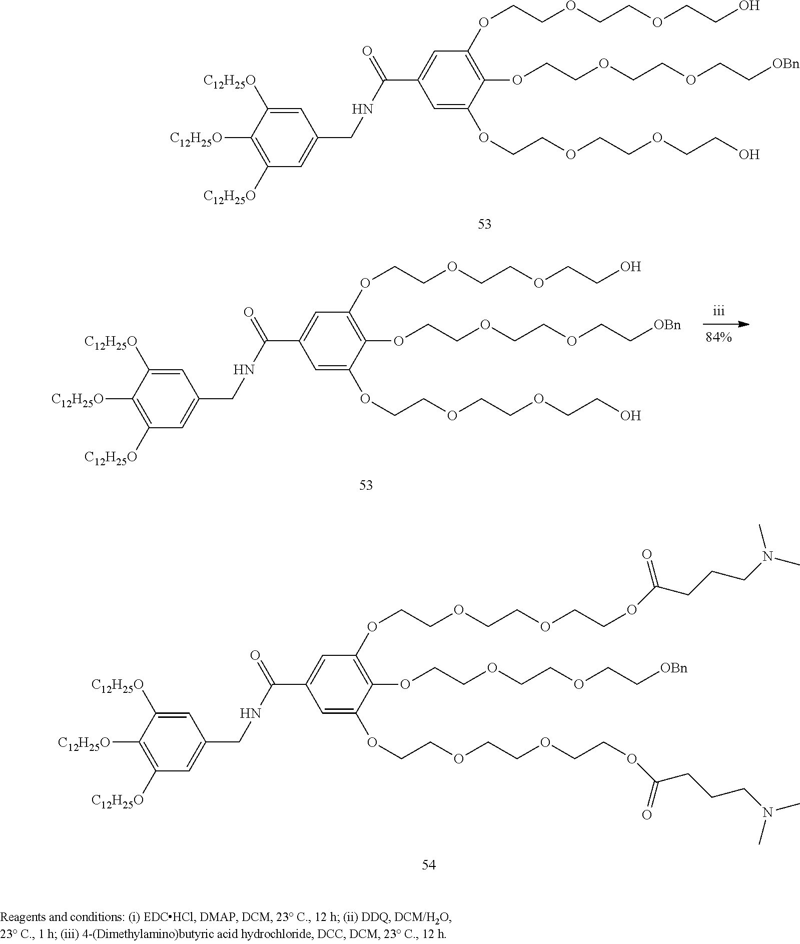

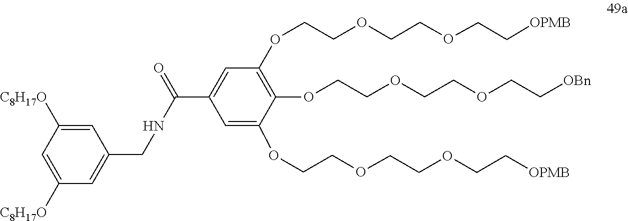

Compound 49a. Compound 9 (0.40 g, 0.45 mmol, 1 equiv), compound 36a (0.18 g, 0.50 mmol, 1.1 equiv), EDC·HCl (96 mg, 0.50 mmol, 1.1 equiv) and DMAP (17 mg, 0.14 mmol, 0.3 equiv) were dissolved in dry DCM (6 mL). The reaction mixture was stirred at 23° C. for 12 h. Brine (30 mL) was added, and the mixture was extracted by DCM (30 mL×3). The organic phase 10 was dried over anhydrous MgSO₄ and filtered. The filtrate was concentrated and purified by column chromatography (SiO₂) with EtOAc/hexane=4/1 as the eluent to give the title compound as a light-yellow oil (0.51 g, 91%).

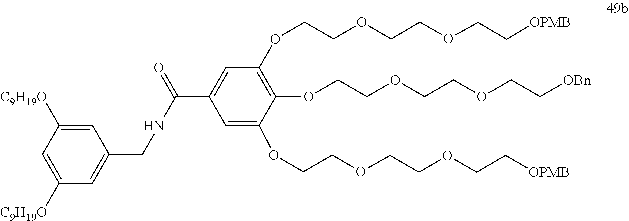

Compound 49b. Compound 49b was synthesized from compound 9 (0.40 g, 0.45 mmol) and compound 36b (0.20 g, 0.50 mmol) following a procedure similar to that used for the synthesis of compound 49a. The title compound was obtained as a yellow oil (0.48 g, 84%).

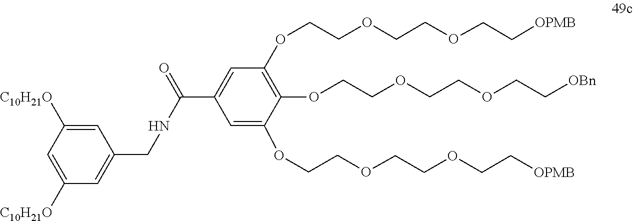

Compound 49c. Compound 49c was synthesized from compound 9 (0.40 g, 0.45 mmol) and compound 36c (0.21 g, 0.50 mmol) following a procedure similar to that used for the synthesis of compound 49a. The title compound was obtained as a yellow oil (0.48 g, 83%).

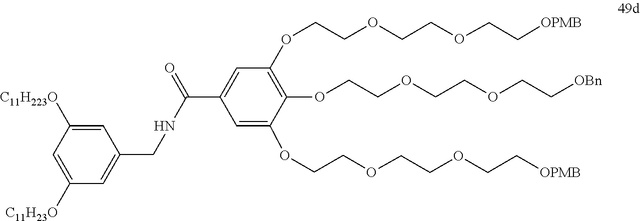

Compound 49d. Compound 49d was synthesized from compound 9 (0.35 g, 0.39 mmol) and compound 36d (0.19 g, 0.43 mmol) following a procedure similar to that used for the synthesis of compound 49a. The title compound was obtained as a light-yellow oil (0.43 g, 83%).

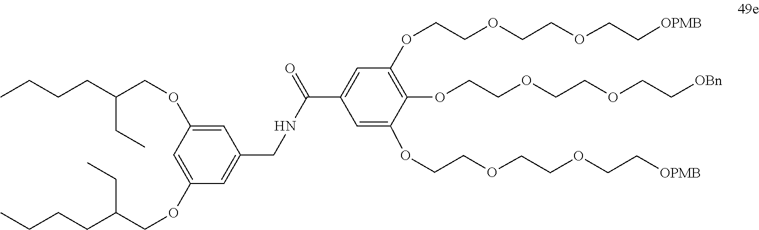

Compound 49e. Compound 49e was synthesized from compound 9 (0.40 g, 0.45 mmol) and compound 36f (0.18 g, 0.50 mmol) following a procedure similar to that used for the synthesis of compound 49a. The title compound was obtained as a light-yellow oil (0.49 g, 88%).

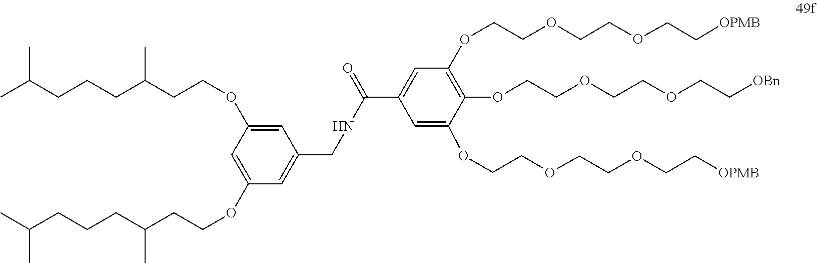

Compound 49f. Compound 49f was synthesized from compound 9 (0.40 g, 0.45 mmol) and compound 36g (0.21 g, 0.50 mmol) following a procedure similar to that used for the synthesis of compound 49a. The title compound was obtained as a light-yellow oil (0.48 g, 85%).

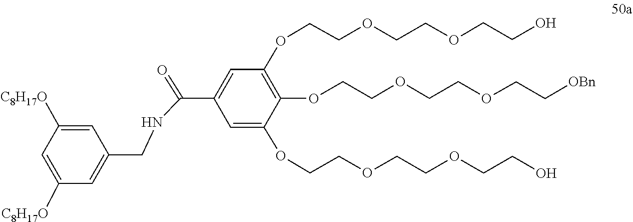

Compound 50a. Compound 49a (0.49 g, 0.39 mmol, 1 equiv) was dissolved in 6 mL DCM and 0.3 mL water (5%) was added. To this solution was added DDQ (0.20 g, 0.86 mmol, 2.2 equiv). The mixture was allowed to stir at 23° C. for 1 h. The precipitates were filtered out and DCM (20 mL) was added. The mixture was washed by NaHCO₃aqueous solution (saturated), NaHSO₃ aqueous solution (2%) and NaHCO₃aqueous solution (saturated) successively. The organic phase was dried over anhydrous MgSO₄ and filtered. The filtrate was concentrated and purified by column chromatography (SiO₂) with EtOAc/MeOH=10/1 as the eluent to give the title compound as a light-yellow oil (0.33 g, 85%).

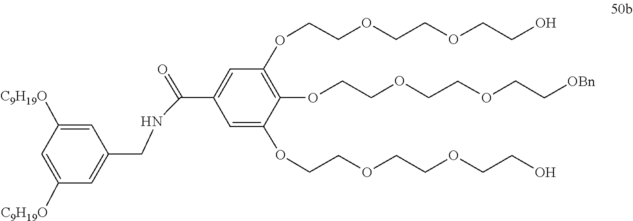

Compound 50b. Compound 50b was synthesized from compound 49b (0.48 g, 0.38 mmol) following a procedure similar to that used for the synthesis of compound 50a. The title compound was obtained as a light-yellow oil (0.32 g, 78%).

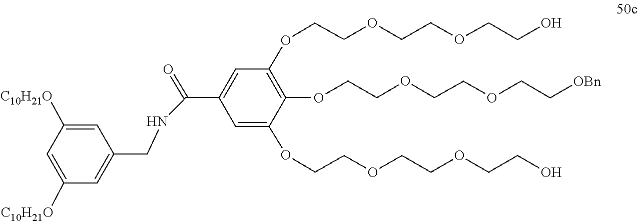

Compound 50c. Compound 50c was synthesized from compound 49c (0.43 g, 0.34 mmol) following a procedure similar to that used for the synthesis of compound 50a. The title compound was obtained as a yellow oil (0.30 g, 86%).

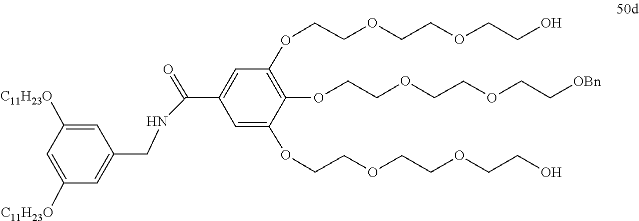

Compound 50d. Compound 50d was synthesized from compound 49d (0.41 g, 0.31 mmol) following a procedure similar to that used for the synthesis of compound 50a. The title compound was obtained as a yellow oil (0.31 g, 91%).

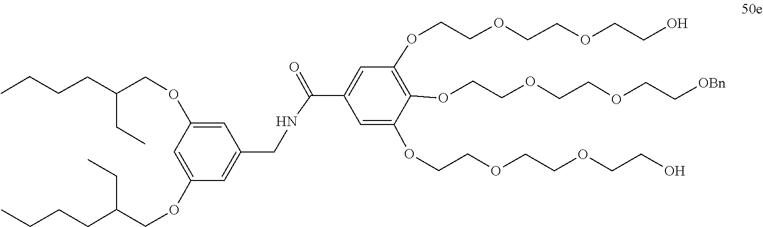 [SMILES: CCCCC(CC)COc1cc(CNC(=O)c2cc(OCCOCCOCCO)c(OCCOCCOCCOCc3ccccc3)c(OCCOCCOCCO)c2)cc(OCC(CC)CCCC)c1]

Compound 50e. Compound 50e was synthesized from compound 49e (0.44 g, 0.35 mmol) following a procedure similar to that used for the synthesis of compound 50a. The title compound was obtained as a yellow oil (0.32 g, 91%).

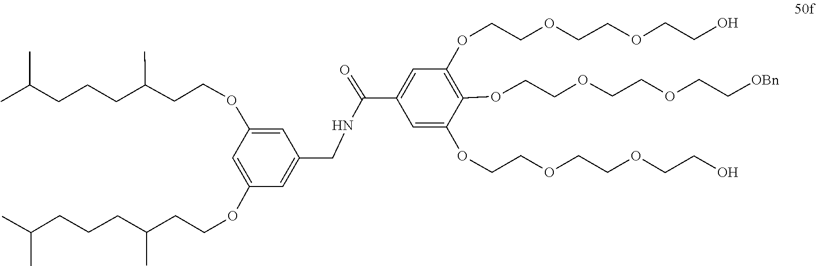 [SMILES: CC(C)CCCC(C)CCOc1cc(CNC(=O)c2cc(OCCOCCOCCO)c(OCCOCCOCCOCc3ccccc3)c(OCCOCCOCCO)c2)cc(OCCC(C)CCCC(C)C)c1]

Compound 50f. Compound 50f was synthesized from compound 49f (0.48 g, 0.37 mmol) following a procedure similar to that used for the synthesis of compound 50a. The title compound was obtained as a yellow oil (0.31 g, 78%).

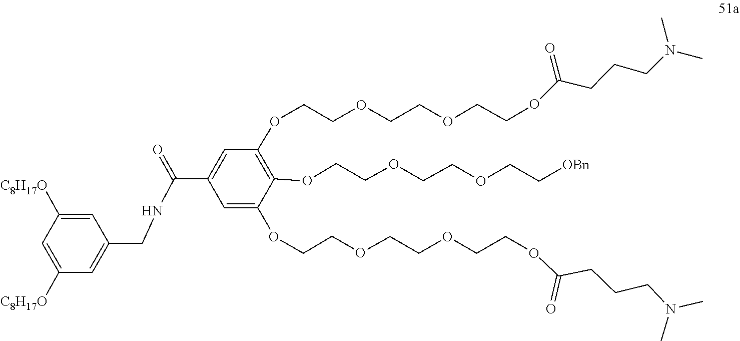

Compound 51a (3/2_{DMBA}^{1,3}_{Bn}²-C₈). Compound ⁵⁰a (0.27 g, 0.27 mmol, 1 equiv) and 4-(dimethylamino)butyric acid hydrochloride (0.10 g, 0.59 mmol, 2.2 equiv) were dissolved in 5 mL dry DCM. DCC (0.17 g, 0.81 mmol, 3 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=10/1, 6/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (0.31 g, 94%).

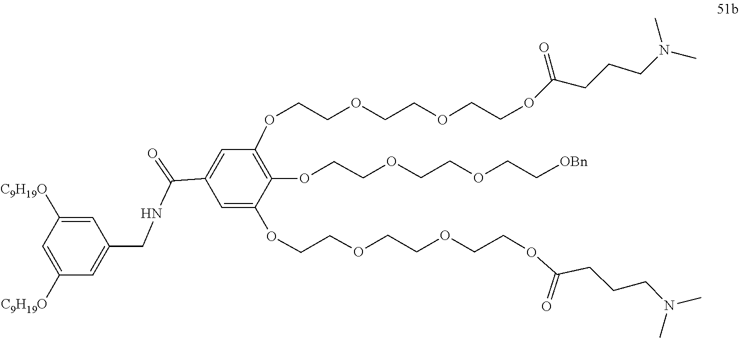

Compound 51b (3/2_{DMBA}^{1,3}_{Bn}²-C9). Compound 51b was synthesized from compound 50b (0.43 g, 0.42 mmol), 4-(dimethylamino)butyric acid hydrochloride (0.15 g, 0.92 mmol) and DCC (0.26 g, 1.26 mmol) following a procedure similar to that used for the synthesis of compound 51a. The title compound was obtained as a light-yellow oil (0.40 g, 82%).

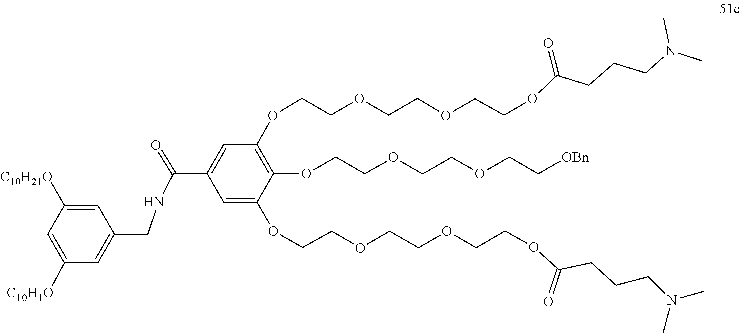

Compound 51c (3/2_{DMBA}^{1,3}_{Bn}²-C10). Compound 51c was synthesized from compound 50c (0.25 g, 0.24 mmol), 4-(dimethylamino)butyric acid hydrochloride (87 mg, 0.52 mmol) and DCC (0.15 g, 0.71 mmol) following a procedure similar to that used for the synthesis of compound 51a. The title compound was obtained as a light-yellow oil (0.26 g, 84%).

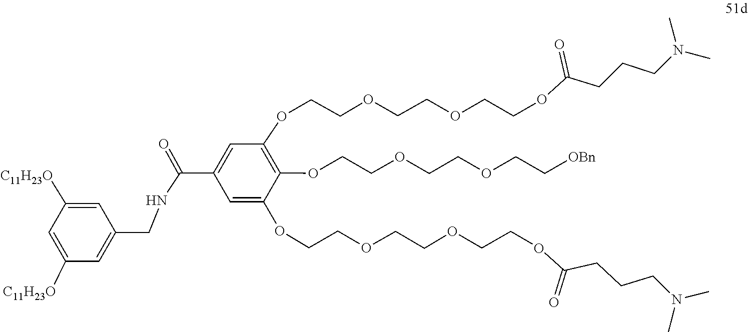

Compound 51d (3/2_{DMBA}^{1,3}_{Bn}²-C11). Compound 51d was synthesized from compound 50d (0.23 g, 0.21 mmol), 4-(dimethylamino)butyric acid hydrochloride (77 mg, 0.46 mmol) and DCC (0.13 g, 0.63 mmol) following a procedure similar to that used for the synthesis of compound 51a. The title compound was obtained as a light-yellow oil (0.23 g, 84%).

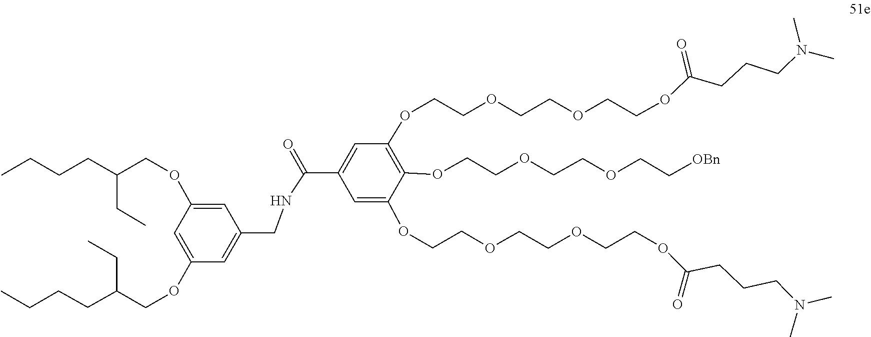 [SMILES: CCCCC(CC)COc1cc(CNC(=O)c2cc(OCCOCCOCCOC(=O)CCCN(C)C)c(OCCOCCOCCOCc3ccccc3)c(OCCOCCOCCOC(=O)CCCN(C)C)c2)cc(OCC(CC)CCCC)c1]

Compound 51e (3/2D_{MB}A1′^{3B}n2-EH). Compound 51e was synthesized from compound 50e (0.29 g, 0.29 mmol), 4-(dimethylamino)butyric acid hydrochloride (0.11 g, 0.64 mmol) and DCC (0.18 g, 0.87 mmol) following a procedure similar to that used for the synthesis of compound 51a. The title compound was obtained as a light-yellow oil (0.32 g, 89%).

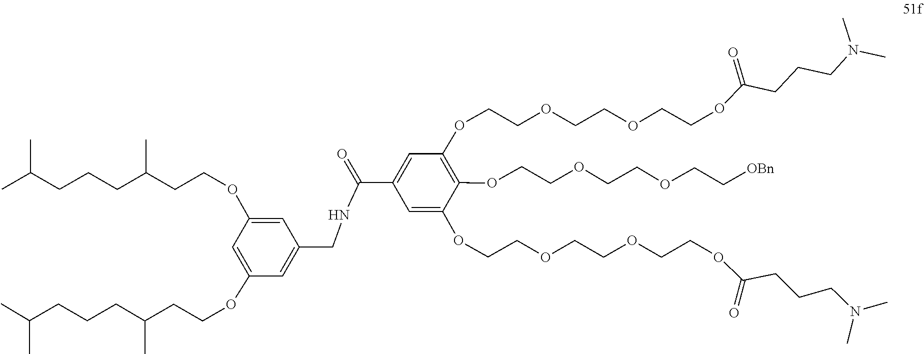 [SMILES: CC(C)CCCC(C)CCOc1cc(CNC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCC(C)CCCC(C)C)c1]

Compound 51f (3/2_{DMBA}^{1,3}_{Bn}²-dm8). Compound 51f was synthesized from compound 50f (0.30 g, 0.28 mmol), 4-(dimethylamino)butyric acid hydrochloride (104 mg, 0.62 mmol) and DCC (0.17 g, 0.84 mmol) following a procedure similar to that used for the synthesis of compound 51a. The title compound was obtained as a light-yellow oil (0.31 g, 86%).

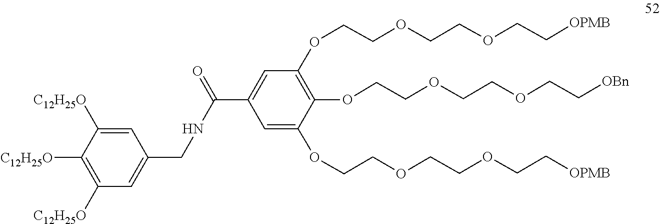

Compound 52. Compound 52 was synthesized from compound 9 (0.30 g, 0.33 mmol) and compound 40 (0.24 g, 0.37 mmol) following a procedure similar to that used for the synthesis of compound 49a. The title compound was obtained as a white viscous solid (0.46 g, 90%).

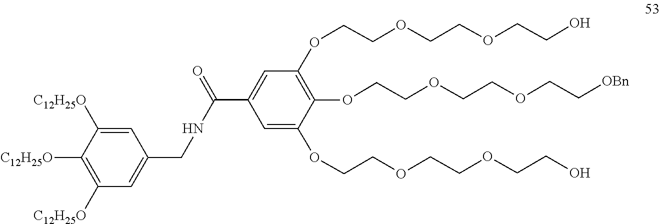

Compound 53. Compound 53 was synthesized from compound 52 (0.45 g, 0.29 mmol) following a procedure similar to that used for the synthesis of compound 50a. The title compound was obtained as a yellow oil (0.23 g, 61%).

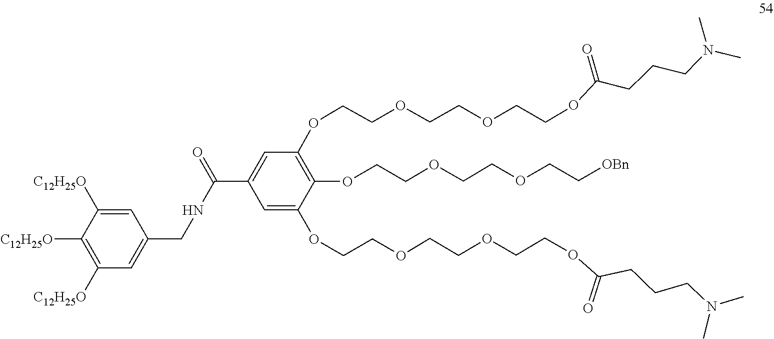

Compound 54 (3/2_{DMBA}^{1,3}_{Bn}²-3,4,5-C₁₂). Compound 54 was synthesized from compound 53 (0.21 g, 0.16 mmol), 4-(dimethylamino)butyric acid hydrochloride (60 mg, 0.36 mmol) and DCC (0.10 g, 0.49 mmol) following a procedure similar to that used for the synthesis of compound 51a. The title compound was obtained as a light-yellow solid (0.21 g, 84%). 15 Synthesis of Single-Single IAJDs in Library 3

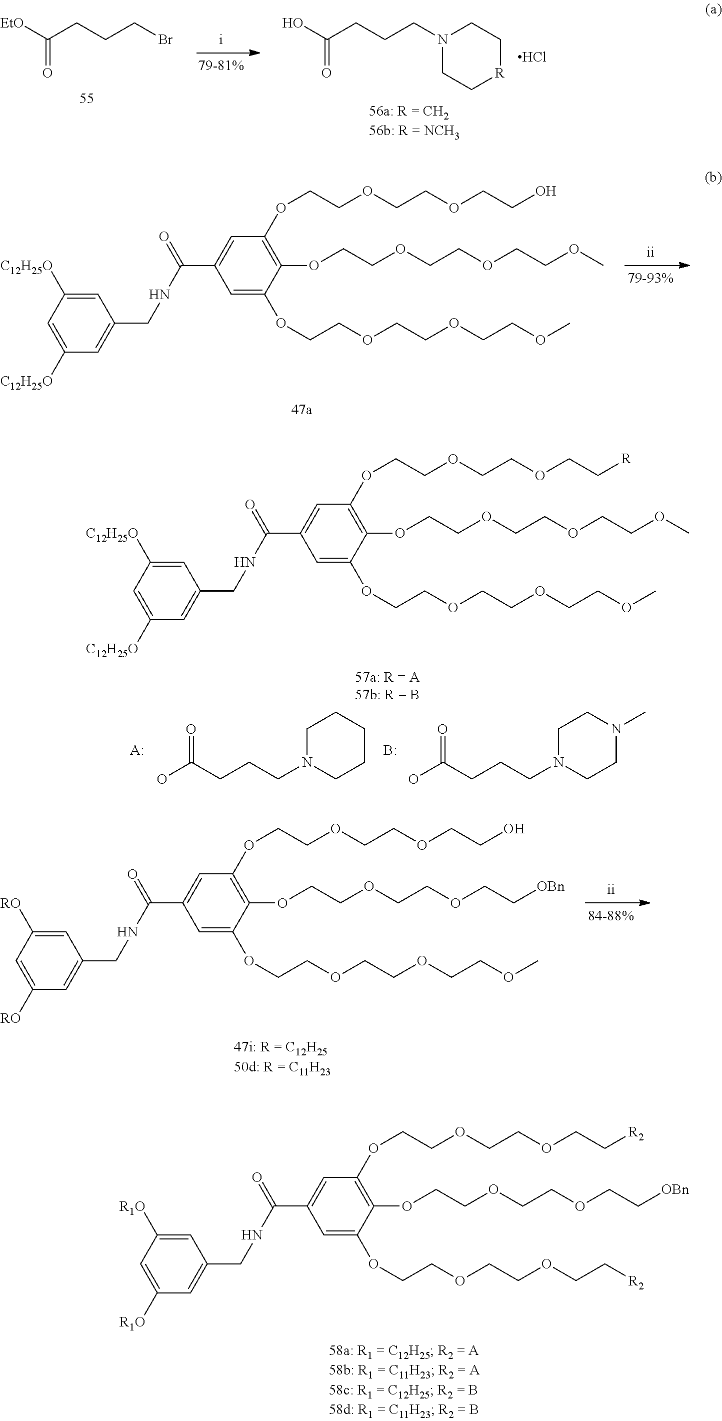

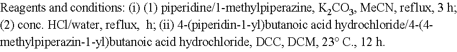

The synthesis of compounds 56a and 56b was adapted from literature procedure (May, et al., Bioconjugate Chem. 2016, 27, 226-237).

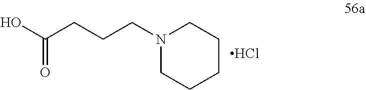 [SMILES: O=C(O)CCCN1CCCCC1 ; Cl]

4-(Piperidin-1-yl)butanoic acid hydrochloride (56a). Ethyl 4-bromobutanoate (55, 1.00 g, 5.15 mmol, 1 equiv), piperidine (0.46 g, 5.41 mmol, 1.05 equiv) and K₂CO₃ (0.70 g, 5.15 mmol, 1 equiv) were stirred in MeCN (20 mL). The mixture was heated at reflux for 3h. The mixture was cooled and extracted by DCM (30 mL×3). The organic phase was washed with NaHCO₃aqueous solution (saturated) (20 mL×2), dried over anhydrous MgSO₄ and filtered. The solvent was evaporated to leave an orange precipitate. The precipitate was suspended in conc. HCl/water (1/1, 10 mL) and the mixture was heated at reflux for 3 h. The solvent was evaporated to give the crude product, which was recrystallized in EtOH to give the title compound as a white solid (0.86 g, 81%).

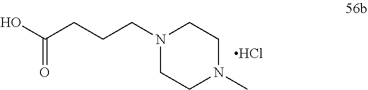

4-(4-Methylpiperazin-1-yl)butanoic acid hydrochloride (56b). Compound 56b was synthesized from compound 55 (1.50 g, 7.73 mmol) and 1-methylpiperazine (0.81 g, 8.12 mmol) following a procedure similar to that used for the synthesis of compound 56a. The title compound was obtained as a white solid (1.31 g, 79%).

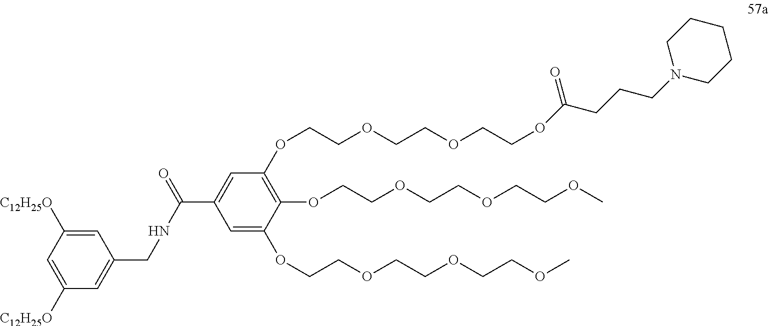

Compound 57a (3/1_{PIP}¹). Compound 57a was synthesized from compound 47a (0.25 g, 0.24 mmol), 4-(piperidin-1-yl)butanoic acid hydrochloride (56a, 0.11 g, 0.53 mmol) and DCC (0.15 g, 0.72 mmol) following a procedure similar to that used for the synthesis of compound 51a. The title compound was obtained as a light-yellow oil (0.23 g, 79%).

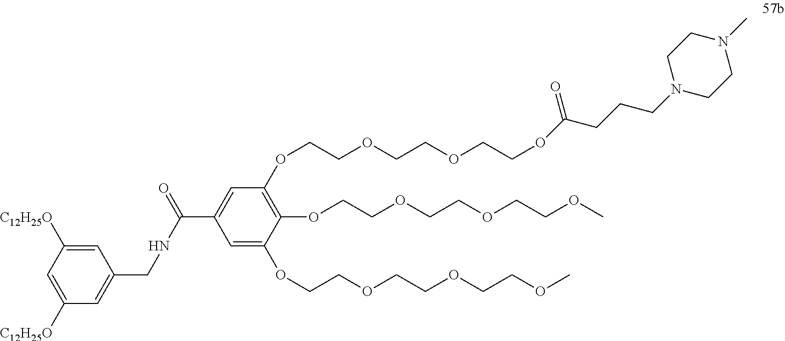

Compound 57b (3/I_{MPRZ}¹). Compound 57b was synthesized from compound 47a (0.25 g, 0.24 mmol), 4-(4-methylpiperazin-1-yl)butanoic acid hydrochloride (56b, 0.12 g, 0.53 mmol) and DCC (0.15 g, 0.72 mmol) following a procedure similar to that used for the synthesis of compound 51a. The title compound was obtained as a light-yellow oil (0.27 g, 93%).

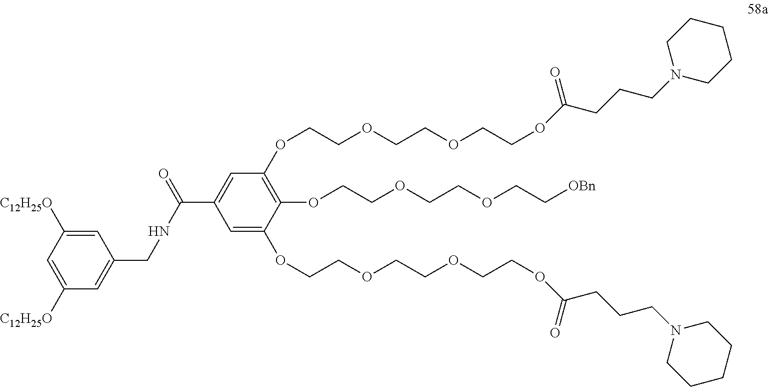

Compound 58a (3/2_{PIP}^{1,3}_{Bn}²). Compound 58a was synthesized from compound 47i (0.30 g, 0.27 mmol), compound 56a (0.12 g, 0.59 mmol) and DCC (0.17 g, 0.81 mmol) following a procedure similar to that used for the synthesis of compound 51a. The title compound was obtained as a light-yellow oil (0.32 g, 84%).

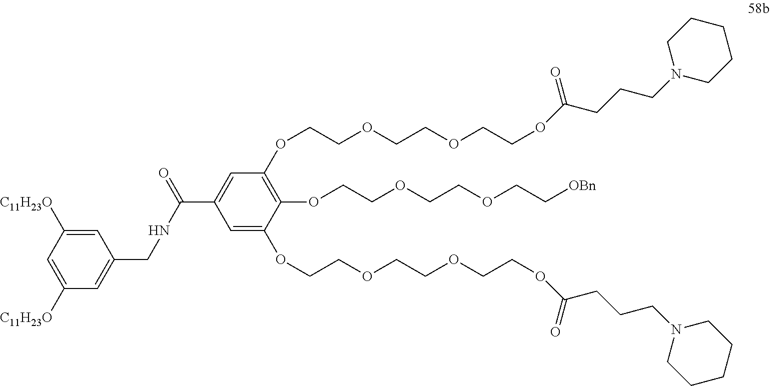

Compound 58b (3/2_{PIP}^{1,3}_{Bn}²-C11). Compound 58b was synthesized from compound 50d (0.28 g, 0.26 mmol), compound 56a (0.11 g, 0.57 mmol) and DCC (0.16 g, 0.78 mmol) following a procedure similar to that used for the synthesis of compound 51a. The title compound was obtained as a light-yellow oil (0.31 g, 86%).

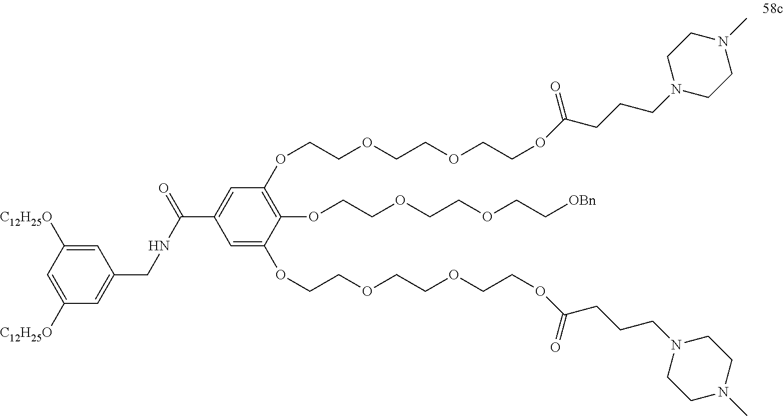

Compound 58c (3/2_{MPRZ}^{1,3}_{Bn}²). Compound 47i (0.30 g, 0.27 mmol, 1 equiv) and compound 56b (0.15 g, 0.68 mmol, 2.5 equiv) were dissolved in 6 mL dry DCM. DCC (0.17 g, 0.81 mmol, 3 equiv) was added in one portion into the above mixture. To the reaction mixture was added 3 drops of TEA. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=10/1, 8/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (0.33 g, 85%).

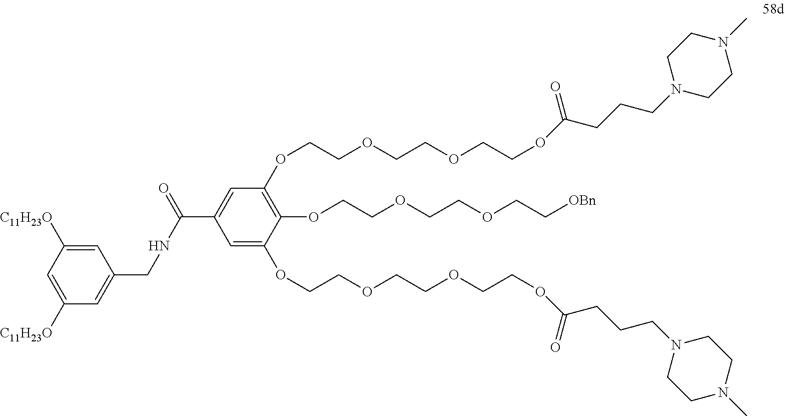

Compound 58d (3/2_{MPRZ}^{1,3}_{Bn}²-C11). Compound 58d was synthesized from compound 50d (0.30 g, 0.28 mmol), compound 56b (0.14 g, 0.62 mmol) and DCC (0.17 g, 0.84 mmol) following a procedure similar to that used for the synthesis of compound 58c. The title compound was obtained as a light-yellow oil (0.35 g, 88%).

### Synthesis of Single-Single IAJDs in Library 4

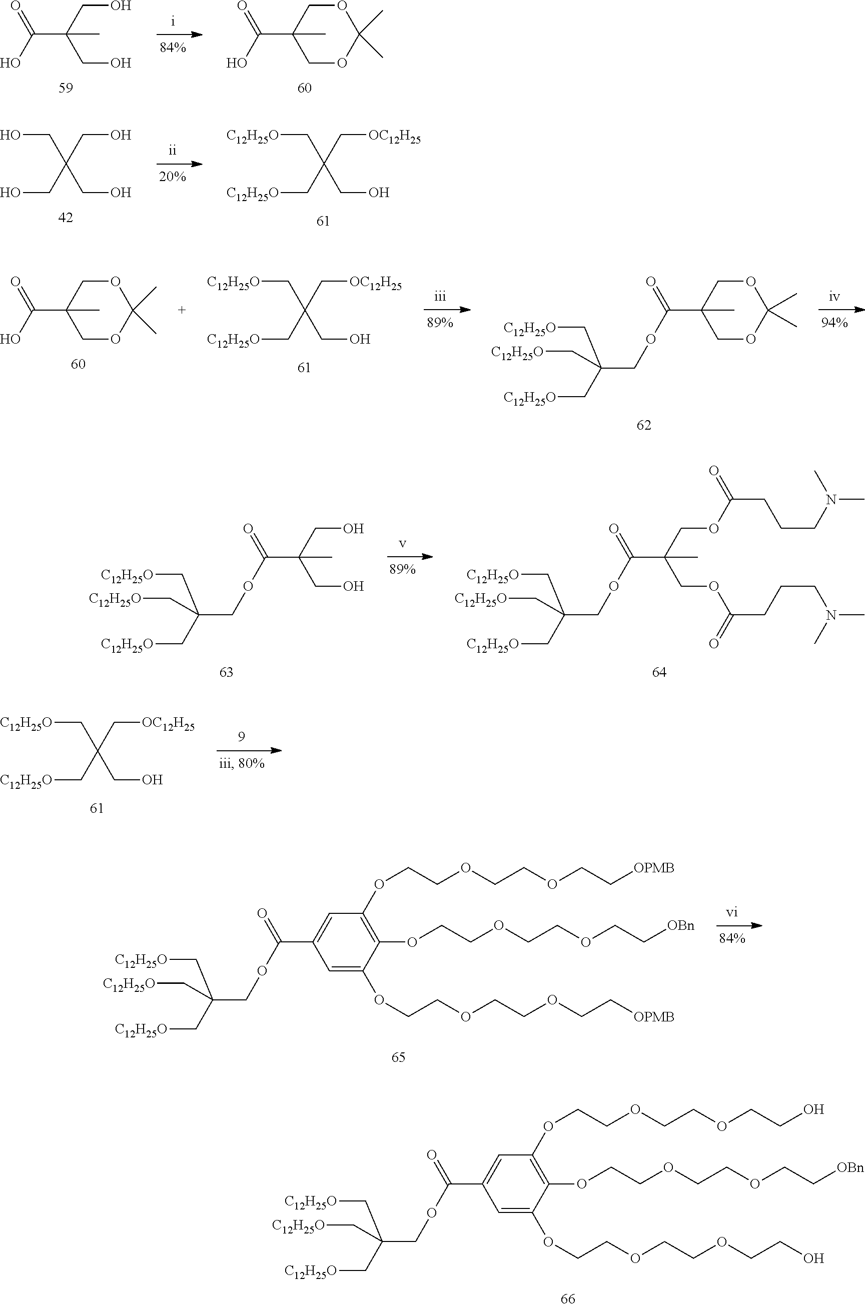

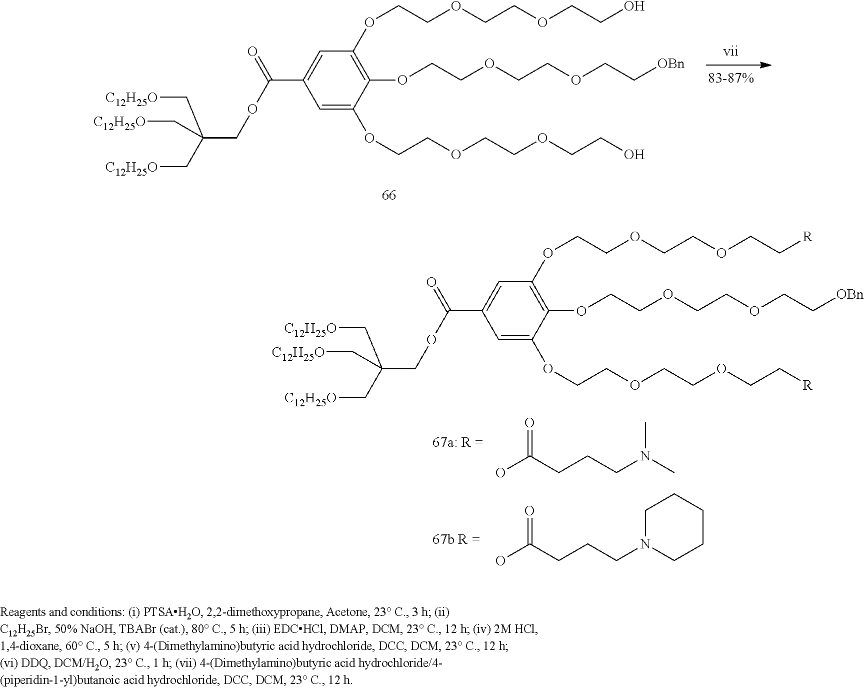

Compounds 60 and 61 were synthesized according to literature procedures The procedures were optimized to increase the yields of compounds 60 and 61 to 10 g and 5 g, respectively.

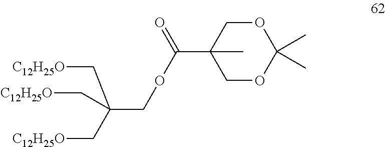

Compound 62. Compound 60 (0.22 g, 1.25 mmol, 1 equiv), compound 61 (0.80 g, 1.25 mmol, 1 equiv), EDC·HCl (0.26 g, 1.38 mmol, 1.1 equiv) and DMAP (46 mg, 0.38 mmol, 0.3 equiv) were dissolved in dry DCM (6 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with hexane/EtOAc=30/1 as the eluent to give the title compound as a colorless oil (0.89 g, 89%).

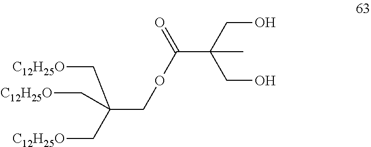

Compound 63. Compound 62 (0.55 g, 0.69 mmol) was dissolved in 20 mL 1,4-dioxane. Then HCl (2 M, 3.0 mL, 6.0 mmol) was added. The mixture was stirred at 60° C. for 5 h. The reaction mixture was extracted by DCM (3×20 mL) and the organic phase was dried over anhydrous MgSO₄. The filtrate was concentrated and purified by column chromatography (SiO₂) with hexane/EtOAc=5/1 as the eluent to give the title compound as a colorless oil (0.49 g, 94%).

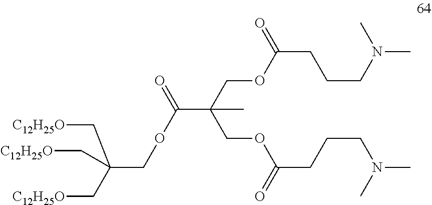

Compound 64 (2/2_{DMBA}^{1,2}). Compound 63 (0.20 g, 0.26 mmol, 1 equiv) and 4-(dimethylamino)butyric acid hydrochloride (96 mg, 0.57 mmol, 2.2 equiv) were dissolved in 5 mL dry DCM. DCC (0.16 g, 0.78 mmol, 3 equiv) was added in one portion into the above mixture. The reaction was allowed to stir at 23° C. for 12 h. The reaction mixture was filtered to remove the urea, 20 which was washed with DCM carefully. The filtrate was concentrated and purified by column chromatography (SiO₂) with DCM/MeOH=10/1, 6/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a colorless oil (0.23 g, 89%).

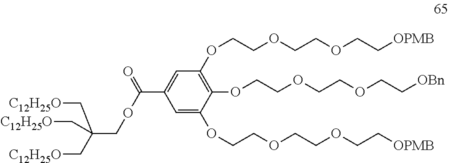

Compound 65. Compound 61 (0.30 g, 0.47 mmol, 1.1 equiv), compound 9 (0.38 g, 0.43 mmol, 1 equiv), EDC·HCl (90 mg, 0.47 mmol, 1.1 equiv) and DMAP (16 mg, 0.13 mmol, 0.3 equiv) were dissolved in dry DCM (6 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (0.52 g, 80%).

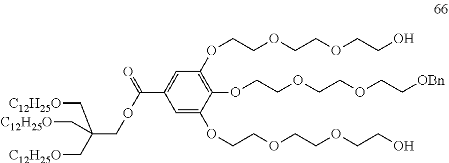

Compound 66. Compound 65 (0.36 g, 0.24 mmol, 1 equiv) was dissolved in 6 mL DCM and 0.3 mL water (5%) was added. To this solution was added DDQ (0.12 g, 0.53 mmol, 2.2 equiv). The mixture was allowed to stir at 23° C. for 1 h. The precipitates were filtered out and DCM (20 mL) was added. The mixture was washed by NaHCO₃aqueous solution (saturated), NaHSO₃ aqueous solution (2%) and NaHCO₃aqueous solution (saturated) successively. The organic phase was dried over anhydrous MgSO₄ and filtered. The filtrate was concentrated and purified by column chromatography (SiO₂) with EtOAc/MeOH=10/1 as the eluent to give the title compound as a light-yellow oil (0.26 g, 84%).

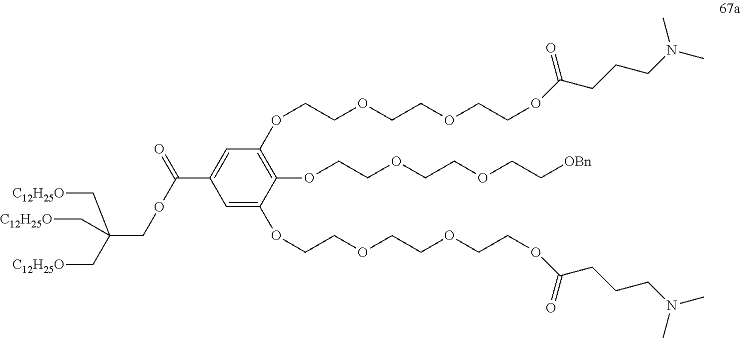

Compound 67a (3/2_{DMBA}^{1,3}_{Bn}²-PE-3C12). Compound 66 (0.25 g, 0.20 mmol, 1 equiv) and 4-(dimethylamino)butyric acid hydrochloride (74 mg, 0.44 mmol, 2.2 equiv) were dissolved in 5 mL dry DCM. DCC (0.13 g, 0.60 mmol, 3 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=10/1, 5/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a colorless oil (0.26 g, 87%).

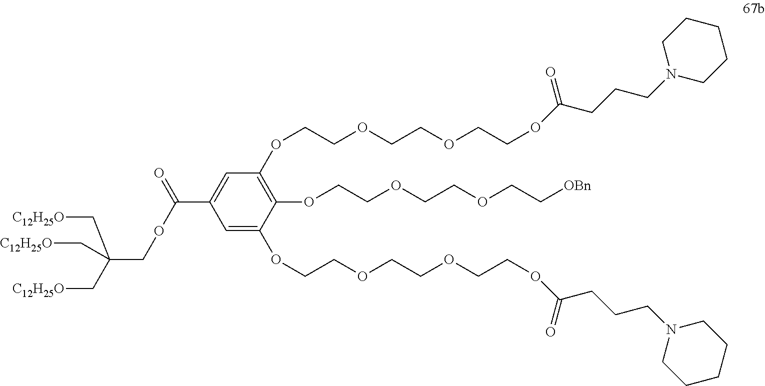

Compound 67b (3/2_{PIP}^{1,3}_{Bn}²-PE-3C12). Compound 67b was synthesized from compound 66 (0.24 g, 0.19 mmol), compound 56a (87 mg, 0.42 mmol) and DCC (0.12 g, 0.57 mmol) following a procedure similar to that used for the synthesis of compound 67a. The title compound was obtained as a colorless oil (0.25 g, 83%).

### Synthesis of Twin-Twin IAJDs of Library 5

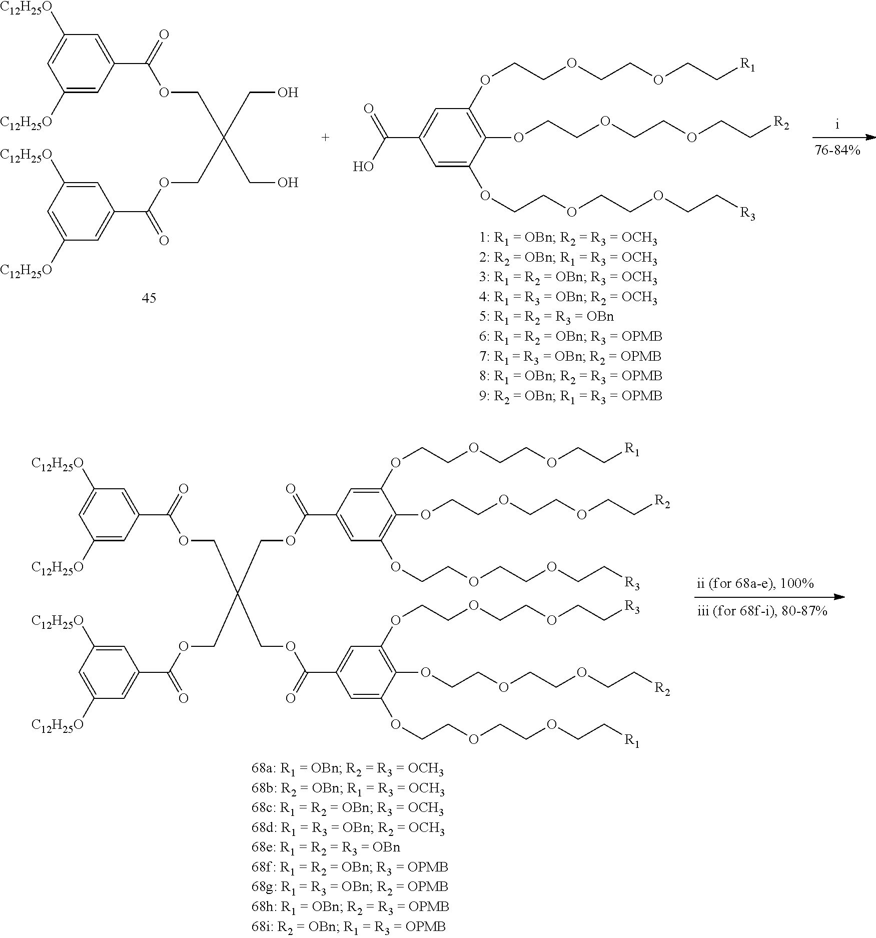

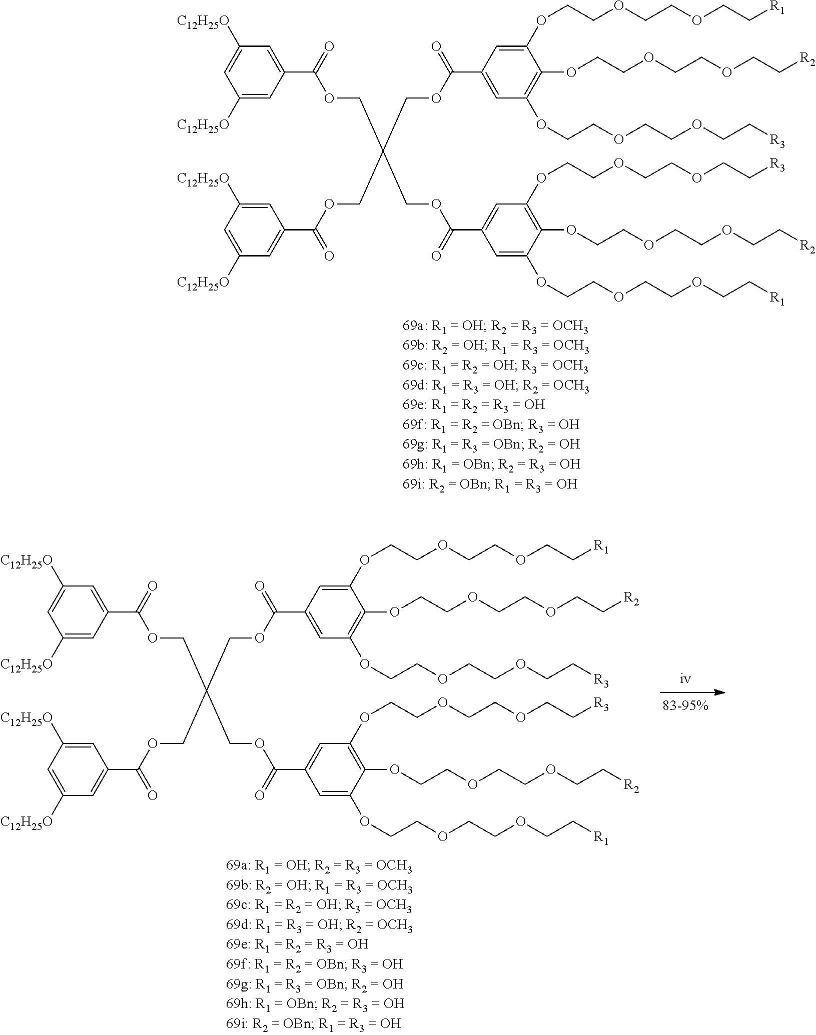

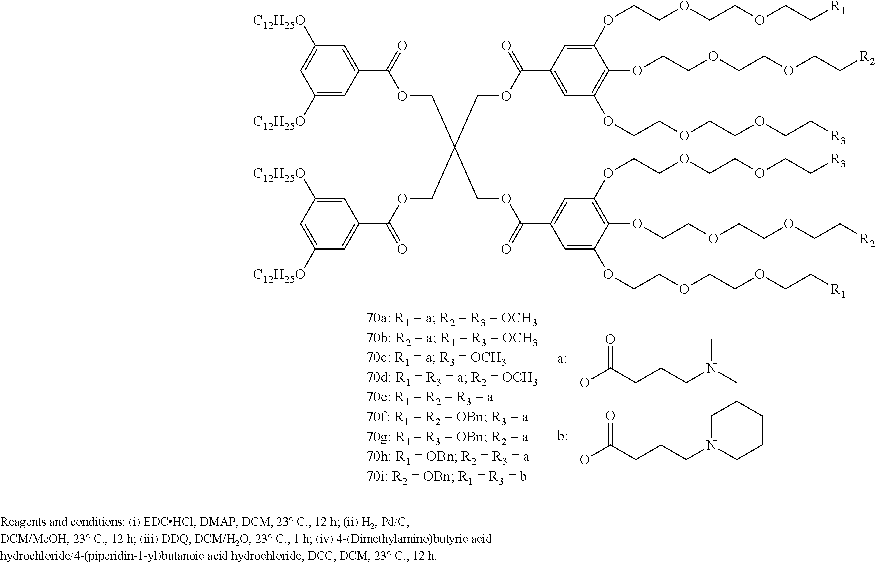

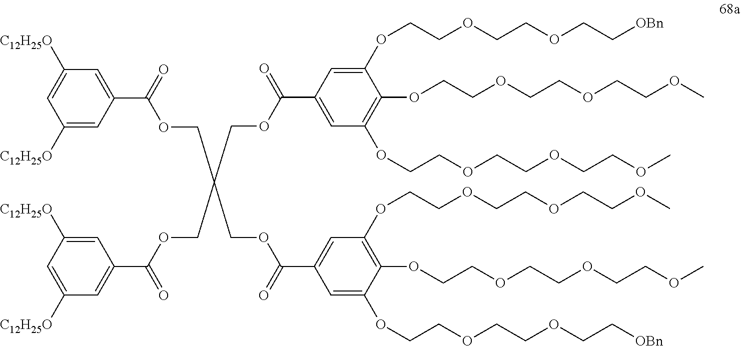

Compound 68a. Compound 45 (0.23 g, 0.21 mmol, 1 equiv), compound 1 (0.30 g, 0.44 mmol, 2.1 equiv), EDC·HCl (84 mg, 0.44 mmol, 2.1 equiv) and DMAP (16 mg, 0.13 mmol, 0.6 equiv) were dissolved in dry DCM (5 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/MeOH=30/1 as the eluent to give the title compound as a light-yellow oil (0.41 g, 81%).

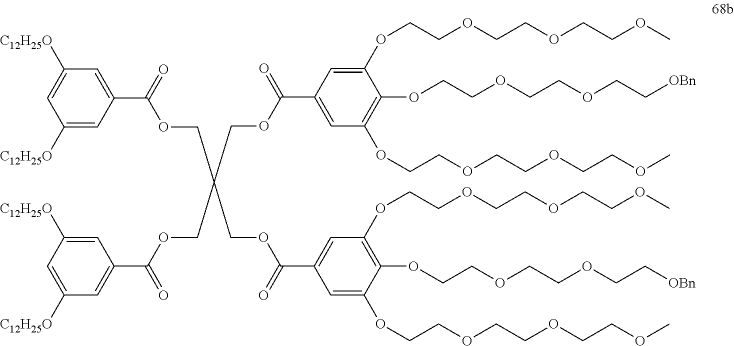

Compound 68b. Compound 45 (0.23 g, 0.21 mmol, 1 equiv), compound 2 (0.30 g, 0.44 mmol, 2.1 equiv), EDC·HCl (84 mg, 0.44 mmol, 2.1 equiv) and DMAP (16 mg, 0.13 mmol, 0.6 equiv) were dissolved in dry DCM (5 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/MeOH=30/1 as the eluent to give the title compound as a light-yellow oil (0.39 g, 77%).

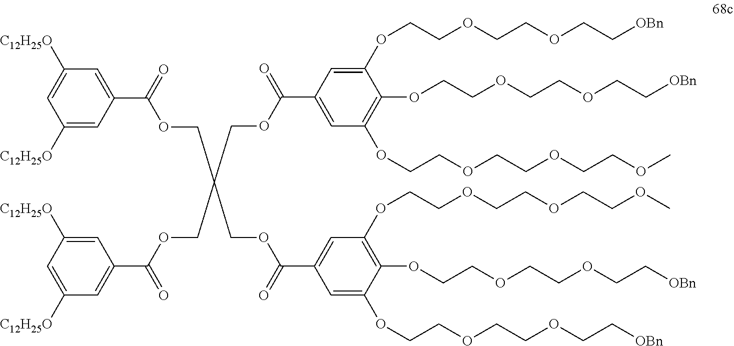

Compound 68c. Compound 45 (0.21 g, 0.19 mmol, 1 equiv), compound 3 (0.30 g, 0.40 mmol, 2.1 equiv), EDC·HCl (77 mg, 0.40 mmol, 2.1 equiv) and DMAP (14 mg, 0.11 mmol, 0.6 equiv) were dissolved in dry DCM (5 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc as the eluent to give the title compound as a colorless oil (0.41 g, 84%).

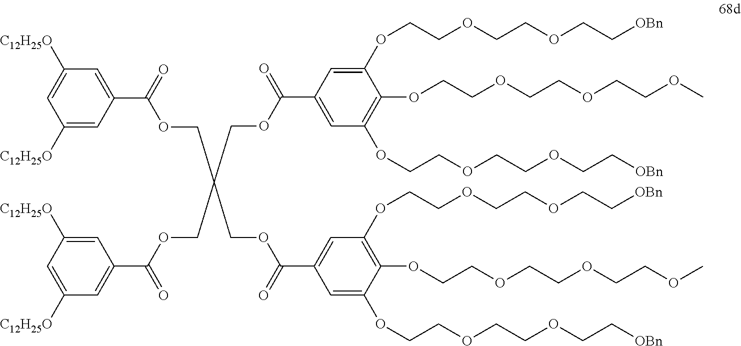

Compound 68d. Compound 45 (0.21 g, 0.19 mmol, 1 equiv), compound 4 (0.30 g, 0.40 mmol, 2.1 equiv), EDC·HCl (77 mg, 0.40 mmol, 2.1 equiv) and DMAP (14 mg, 0.11 mmol, 0.6 equiv) were dissolved in dry DCM (5 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc as the eluent to give the title compound as a colorless oil (0.40 g, 82%).

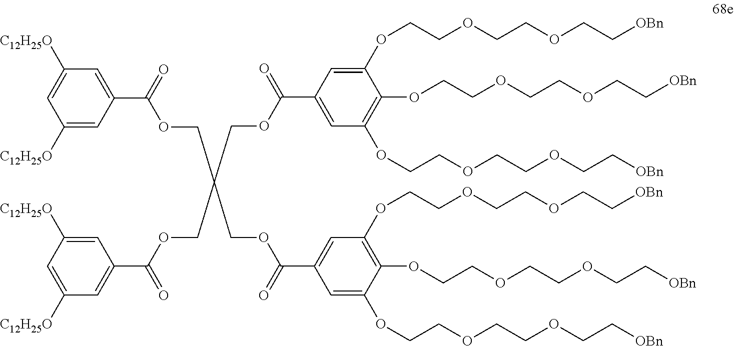

Compound 68e. Compound 45 (0.18 g, 0.17 mmol, 1 equiv), compound 5 (0.30 g, 0.36 mmol, 2.1 equiv), EDC·HCl (69 mg, 0.36 mmol, 2.1 equiv) and DMAP (12 mg, 0.10 mmol, 0.6 equiv) were dissolved in dry DCM (5 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (0.36 g, 78%).

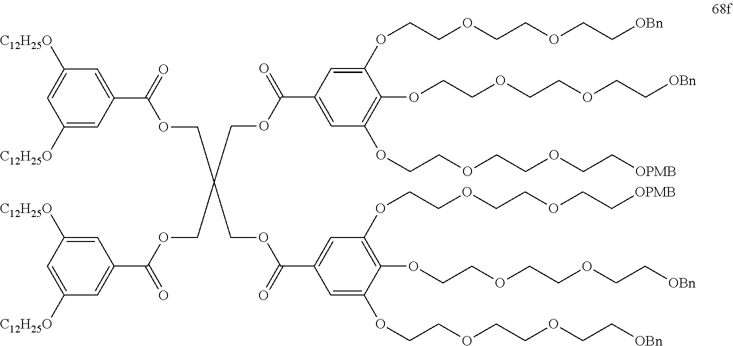

Compound 68f. Compound 45 (0.18 g, 0.17 mmol, 1 equiv), compound 6 (0.30 g, 0.35 mmol, 2.1 equiv), EDC·HCl (67 mg, 0.35 mmol, 2.1 equiv) and DMAP (12 mg, 0.10 mmol, 0.6 equiv) were dissolved in dry DCM (5 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (0.36 g, 77%).

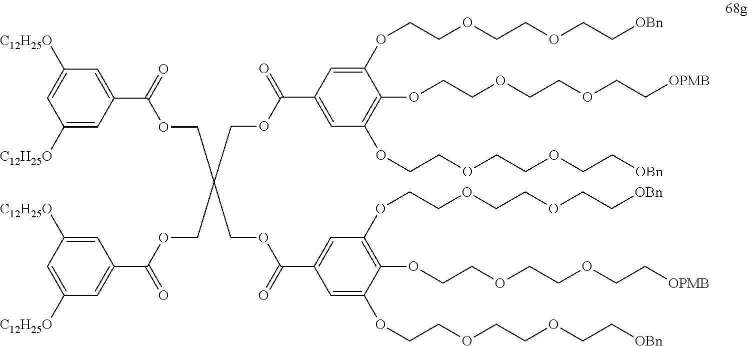

Compound 68g. Compound 45 (0.18 g, 0.17 mmol, 1 equiv), compound 7 (0.30 g, 0.35 mmol, 2.1 equiv), EDC·HCl (67 mg, 0.35 mmol, 2.1 equiv) and DMAP (12 mg, 0.10 mmol, 0.6 equiv) were dissolved in dry DCM (5 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (0.39 g, 84%).

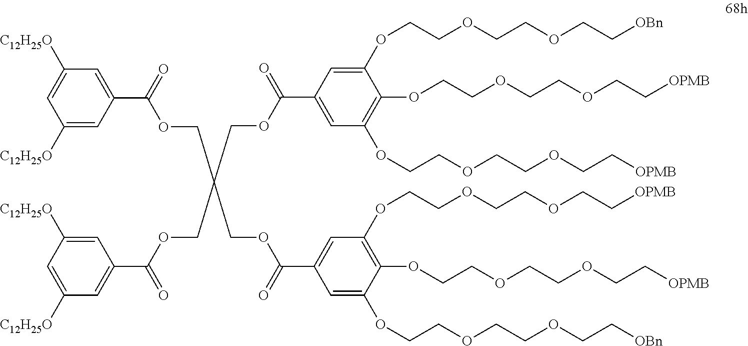

Compound 68h. Compound 45 (0.35 g, 0.32 mmol, 1 equiv), compound 8 (0.60 g, 0.67 mmol, 2.1 equiv), EDC·HCl (0.13 g, 0.67 mmol, 2.1 equiv) and DMAP (24 mg, 0.19 mmol, 0.6 equiv) were dissolved in dry DCM (6 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=4/1 as the eluent to give the title compound as a light-yellow oil (0.68 g, 76%).

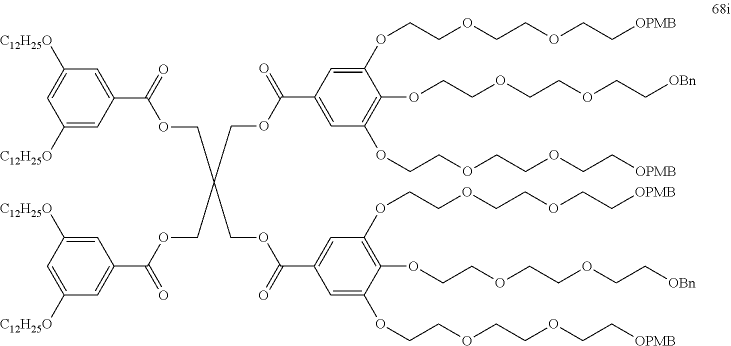

Compound 68i. Compound 45 (0.35 g, 0.32 mmol, 1 equiv), compound 9 (0.60 g, 0.67 mmol, 2.1 equiv), EDC·HCl (0.13 g, 0.67 mmol, 2.1 equiv) and DMAP (24 mg, 0.19 mmol, 0.6 equiv) were dissolved in dry DCM (6 mL). The reaction mixture was stirred at 23° C. for 12 h. Brine (30 mL) was added, and the mixture was extracted by DCM (30 mL×3). The organic phase was dried over anhydrous MgSO₄ and filtered. The filtrate was concentrated and purified by column chromatography (SiO₂) with EtOAc/hexane=3/1 as the eluent to give the title compound as a light-yellow oil (0.72 g, 78%).

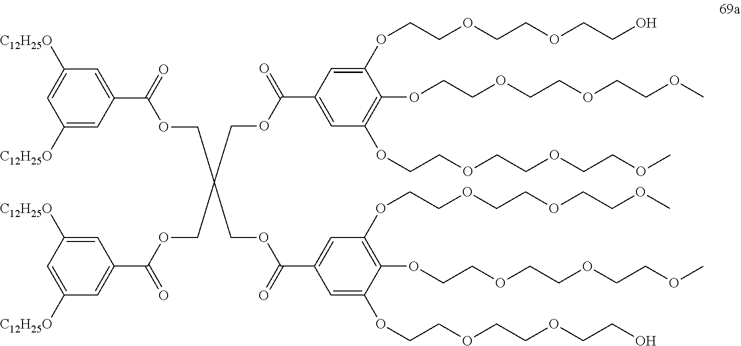

Compound 69a. Compound 68a (0.39 g, 0.16 mmol) was dissolved in 1:2 MeOH:DCM (15 mL). Then Pd/C (19.5 mg, 5 wt %) was added and the flask was evacuated and filled with hydrogen for three times. The mixture was stirred at 23° C. for 12 h under hydrogen atmosphere. The reaction mixture was filtered through Celite and the filter cake was washed with DCM. Evaporation of the solvent yielded the title compound as a light-yellow oil (0.36 g, 100%).

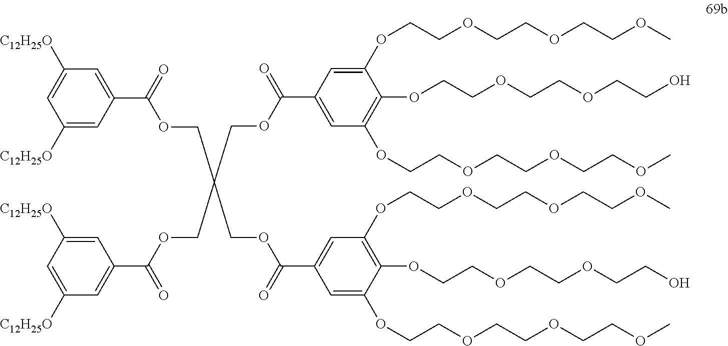

Compound 69b. Compound 69b was synthesized from compound 68b (0.38 g, 0.16 mmol) following a procedure similar to that used for the synthesis of compound 69a. The title compound was obtained as a light-yellow oil (0.36 g, 100%).

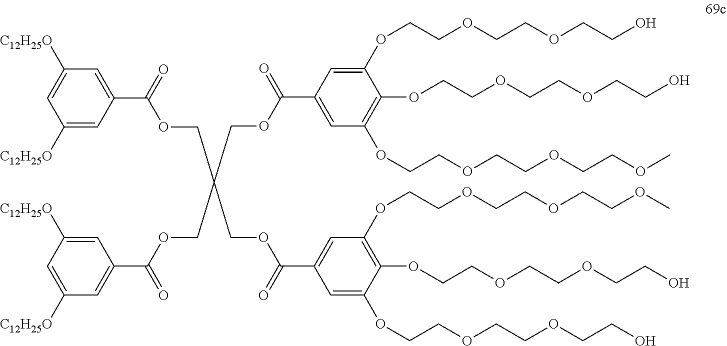

Compound 69c. Compound 69c was synthesized from compound 68c (0.39 g, 0.15 mmol) following a procedure similar to that used for the synthesis of compound 69a. The title compound was obtained as a light-yellow oil (0.33 g, 100%).

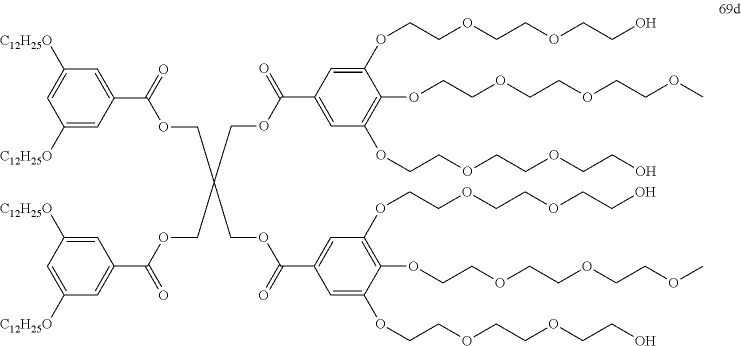

Compound 69d. Compound 69d was synthesized from compound 68d (0.39 g, 0.15 mmol) following a procedure similar to that used for the synthesis of compound 69a. The title compound was obtained as a light-yellow oil (0.33 g, 100%).

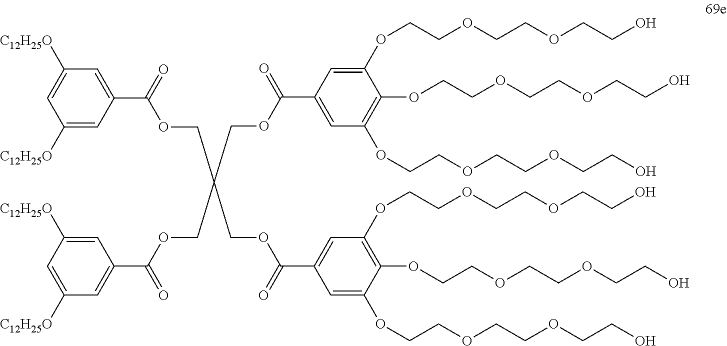

Compound 69e. Compound 69e was synthesized from compound 68e (0.32 g, 0.12 mmol) following a procedure similar to that used for the synthesis of compound 69a. The title compound was obtained as a light-yellow oil (0.26 g, 100%).

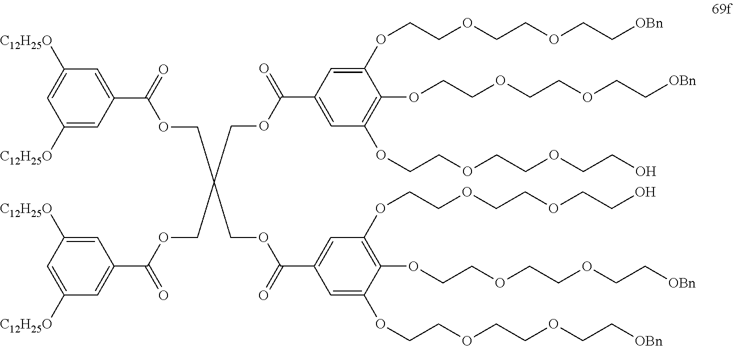

Compound 69f. Compound 68f (0.26 g, 0.09 mmol, 1 equiv) was dissolved in 5 mL DCM and 0.25 mL water (5%) was added. To this solution was added DDQ (45 mg, 0.20 mmol, 2.2 equiv). The mixture was allowed to stir at 23° C. for 1 h. The precipitates were filtered out and DCM (20 mL) was added. The mixture was washed by NaHCO₃aqueous solution (saturated), NaHSO₃ aqueous solution (2%) and NaHCO₃aqueous solution (saturated) successively. The organic phase was dried over anhydrous MgSO₄ and filtered. The filtrate was concentrated and purified by column chromatography (SiO₂) with EtOAc/MeOH=20/1 as the eluent to give the title compound as a light-yellow oil (0.19 g, 83%).

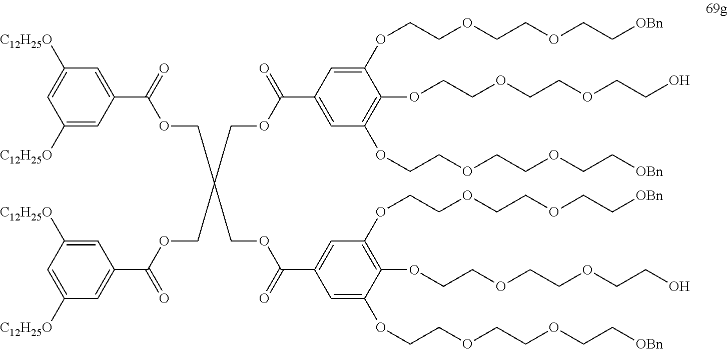

Compound 69g. Compound 69g was synthesized from compound 68g (0.18 g, 0.07 mmol) following a procedure similar to that used for the synthesis of compound 69f. The title compound was obtained as a light-yellow oil (0.13 g, 80%).

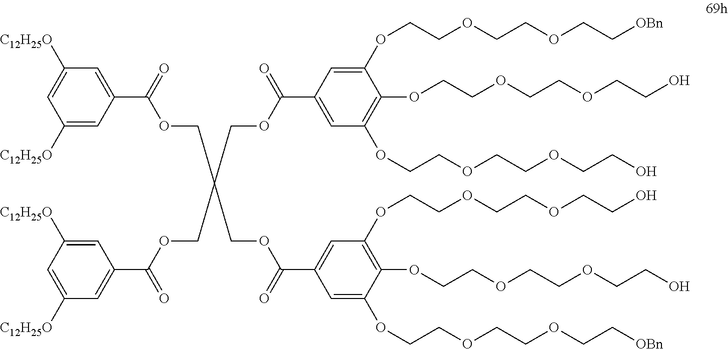

Compound 69h. Compound 69h was synthesized from compound 68h (0.53 g, 0.19 mmol) following a procedure similar to that used for the synthesis of compound 69f. The title compound was obtained as a yellow oil (0.39 g, 87%).

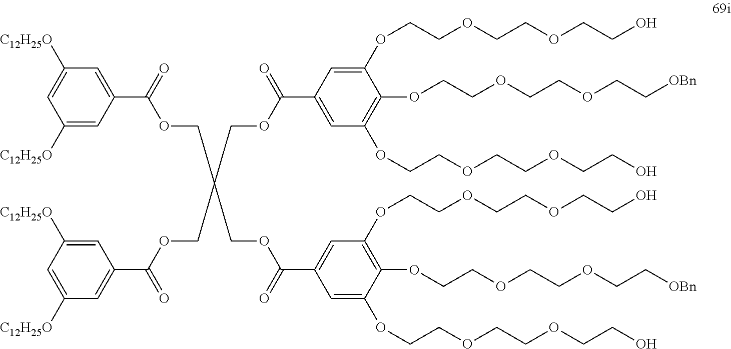

Compound 69i. Compound 69i was synthesized from compound 68i (0.57 g, 0.20 mmol) following a procedure similar to that used for the synthesis of compound 69f. The title compound was obtained as a yellow oil (0.40 g, 84%).

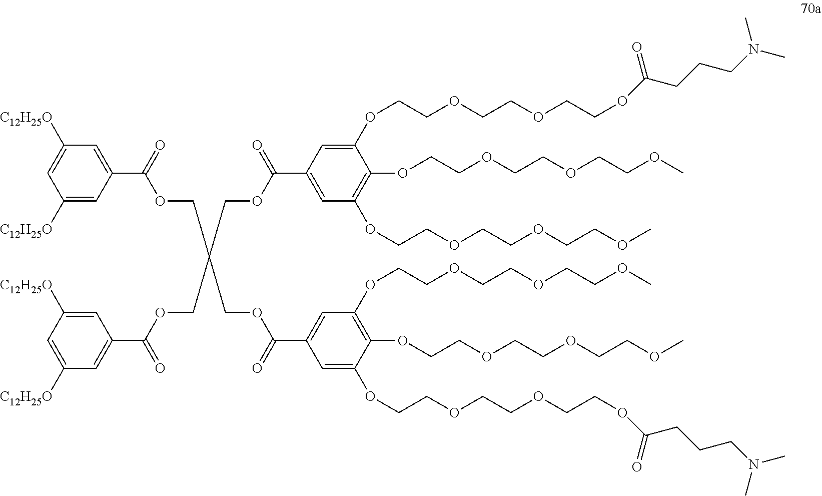

Compound 70a (6/2_{DMBA}^{1,1′}). Compound 69a (0.32 g, 0.14 mmol, 1 equiv) and 4-(dimethylamino)butyric acid hydrochloride (54 mg, 0.32 mmol, 2.3 equiv) were dissolved in 6 mL dry DCM. DCC (87 mg, 0.42 mmol, 3.0 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=8/1, 6/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (0.33 g, 94%).

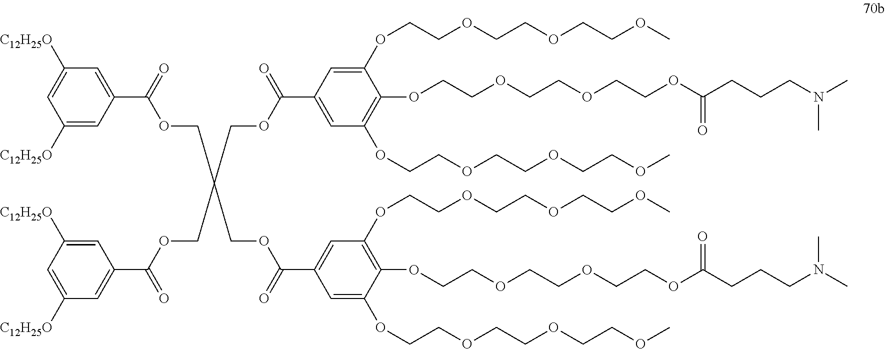

Compound 70b (6/2_{DMBA}²,2′). Compound 69b (0.28 g, 0.13 mmol, 1 equiv) and 4-(dimethylamino)butyric acid hydrochloride (48 mg, 0.29 mmol, 2.3 equiv) were dissolved in 6 mL dry DCM. DCC (77 mg, 0.38 mmol, 3.0 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=8/1, 6/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (0.26 g, 84%).

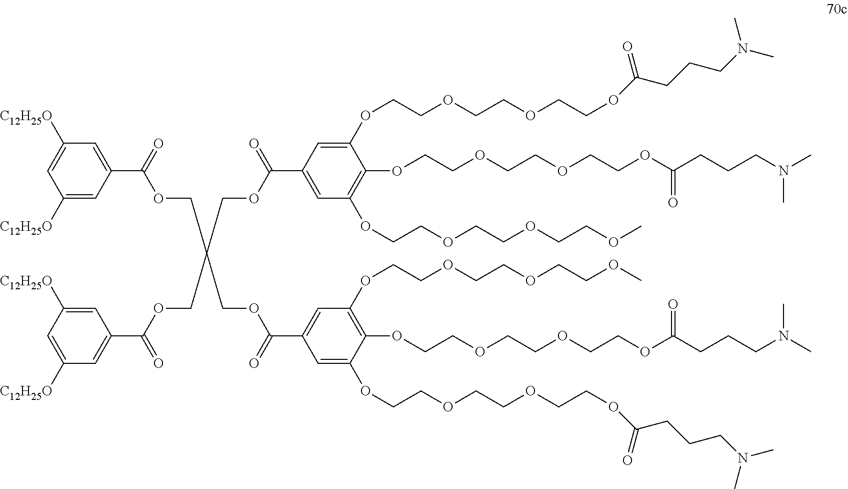

Compound 70c (6/4_{DMBA}^{1,1′2′}). Compound 69c (0.28 g, 0.13 mmol, 1 equiv) and 4-(dimethylamino)butyric acid hydrochloride (0.11 g, 0.65 mmol, 5.0 equiv) were dissolved in 6 mL dry DCM. DCC (0.16 g, 0.78 mmol, 6.0 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=6/1, 3/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (0.29 g, 83%).

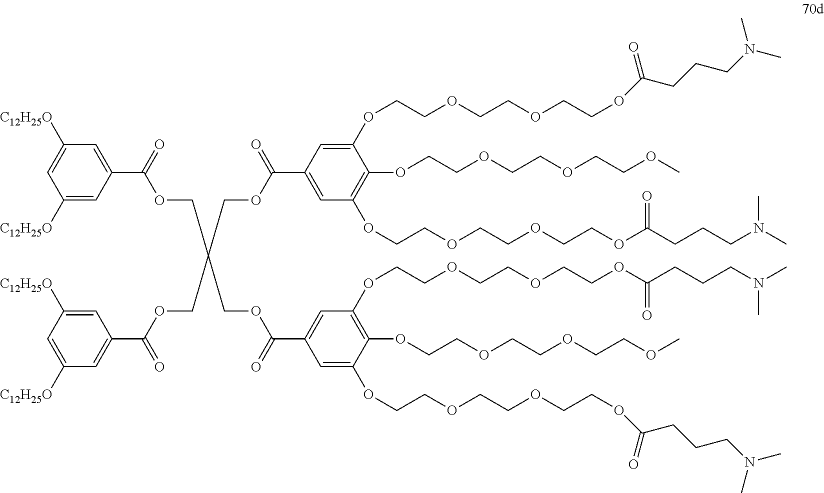

Compound 70d (6/4_{DMBA}^{1,1′3′}). Compound 69d (0.18 g, 0.08 mmol, 1 equiv) and 4-(dimethylamino)butyric acid hydrochloride (69 mg, 0.41 mmol, 5.0 equiv) were dissolved in 5 mL dry DCM. DCC (0.10 g, 0.49 mmol, 6.0 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=6/1, 3/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (0.19 g, 88%).

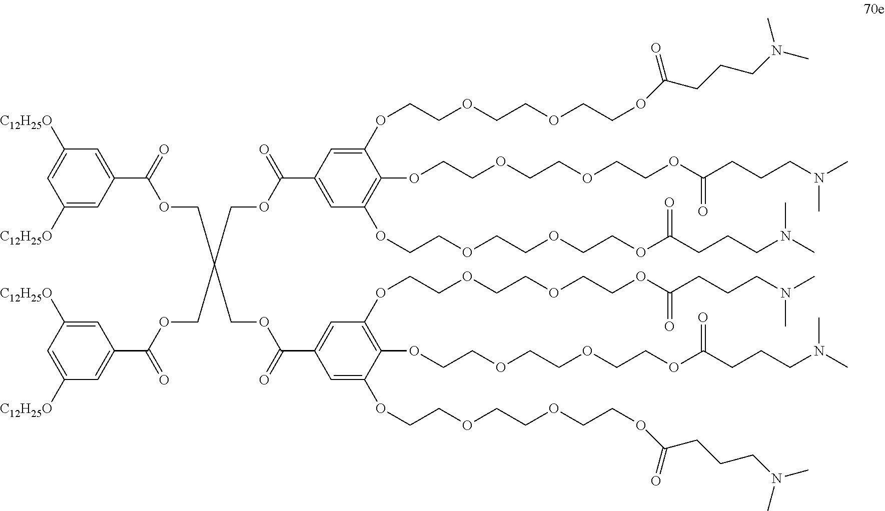

Compound 70e (6/6_{DMBA}^{1,1′,2,2′,3,3′}). Compound 69e (95 mg, 0.04 mmol, 1 equiv) and 4-(dimethylamino)butyric acid hydrochloride (59 mg, 0.35 mmol, 8.0 equiv) were dissolved in 4 mL dry DCM. DCC (90 mg, 0.44 mmol, 10.0 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=6/1, 3/1, 1/1 with 0.2% TEA as the eluent. Then the crude product was dissolved in NaHCO₃solution (2%, 20 mL) and the mixture was extracted by DCM (20 mL×3). The organic phase was dried over anhydrous MgSO₄. Filtration and evaporation of the solvent yielded the title compound as a light-yellow oil (0.11 g, 83%).

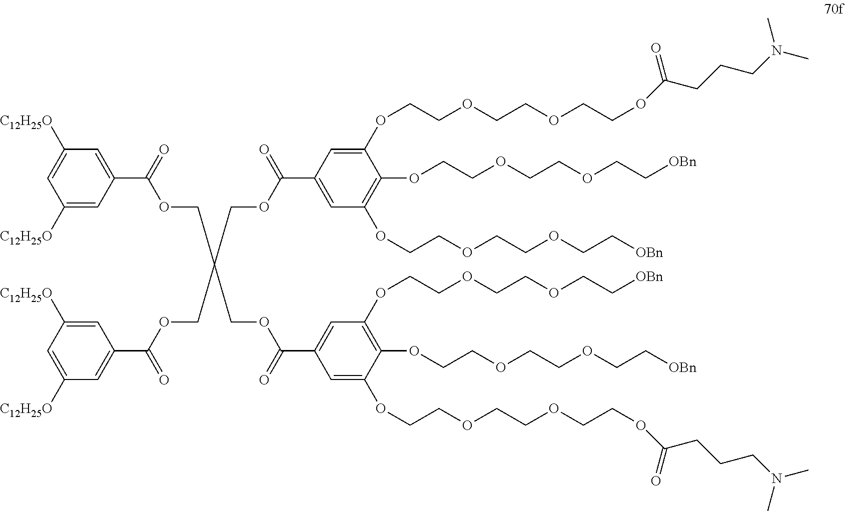

Compound 70f (6/2_{DMBA}^{1,1′}_{Bn}^{2′,2′,3,3′}). Compound 69f (0.15 g, 0.06 mmol, 1 equiv) and 4-(dimethylamino)butyric acid hydrochloride (22 mg, 0.13 mmol, 2.2 equiv) were dissolved in 4 mL dry DCM. DCC (37 mg, 0.18 mmol, 3.0 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=6/1 as the eluent to afford the title compound as a light-yellow oil (0.14 g, 88%).

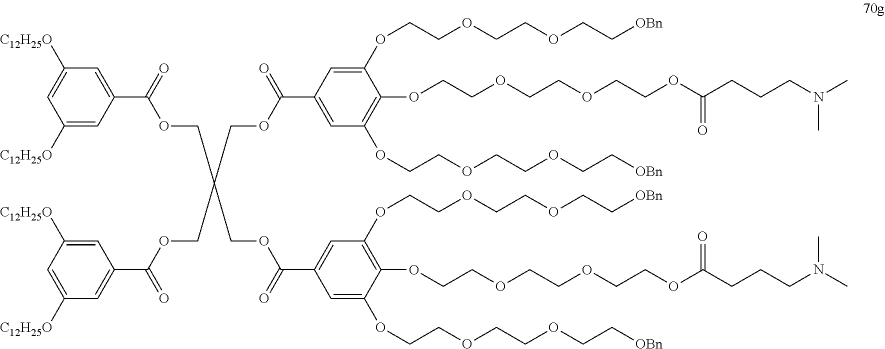

Compound 70g (6/2_{DMBA}^{2′2′}_{Bn}^{1,1′,3,3′}). Compound 69g (95 mg, 0.04 mmol, 1 equiv) and 4-(dimethylamino)butyric acid hydrochloride (15 mg, 0.09 mmol, 2.2 equiv) were dissolved in 4 mL dry DCM. DCC (24 mg, 0.12 mmol, 3.0 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=10/1 as the eluent to afford the title compound as a colorless oil (92 mg, 86%).

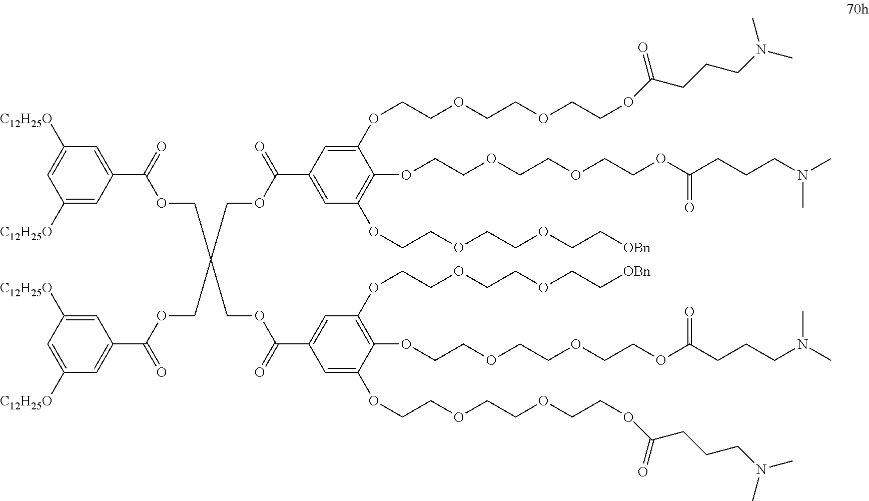

Compound 70h (6/4_{DMBA}^{1,1′, 2,2′}_{Bn}^{3,3′}). Compound 69h (0.31 g, 0.13 mmol, 1 equiv) and 4-(dimethylamino)butyric acid hydrochloride (97 mg, 0.58 mmol, 4.4 equiv) were dissolved in 6 mL 15 dry DCM. DCC (0.16 g, 0.79 mmol, 6.0 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=6/1, 5/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (0.35 g, 95%).

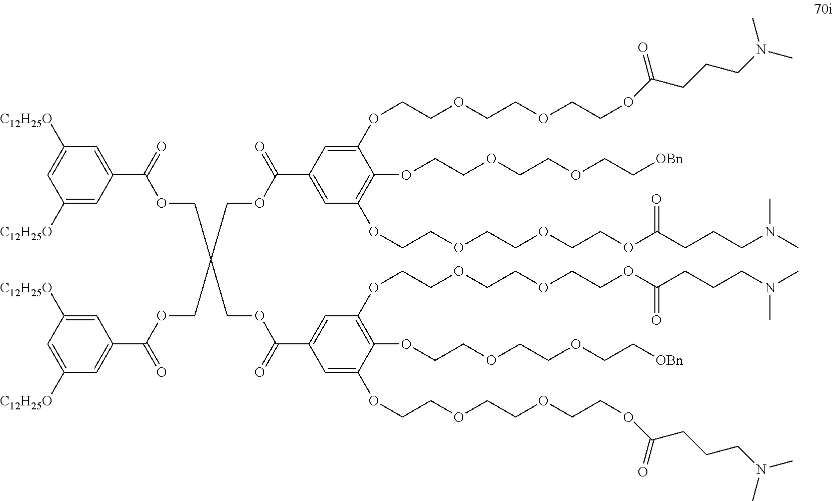

Compound 70i (6/4_{DMBA}^{1,1′,3,3′}_{Bn}^{2,2′}). Compound 69i (0.34 g, 0.14 mmol, 1 equiv) and 4-(dimethylamino)butyric acid hydrochloride (0.11 g, 0.63 mmol, 4.4 equiv) were dissolved in 6 mL dry DCM. DCC (0.18 g, 0.86 mmol, 6.0 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=6/1, 5/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (0.37 g, 89%).

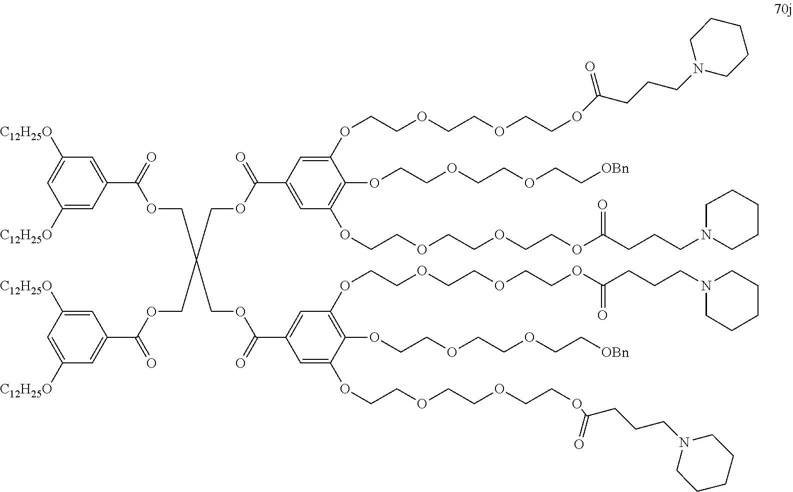

Compound 70j (6/4_{PIP}^{1′,3,3′}_{Bn}^{2,2′}). Compound 70j was synthesized from compound 69i (0.15 g, 0.064 mmol), compound 56a (58 mg, 0.28 mmol) and DCC (79 mg, 0.38 mmol) following a procedure similar to that used for the synthesis of compound 70i. The title compound was obtained as a light-yellow oil (0.17 g, 90%).

### 3.7 Synthesis of Hybrid Twin-Mix IAJDs 38-42 of Library 6

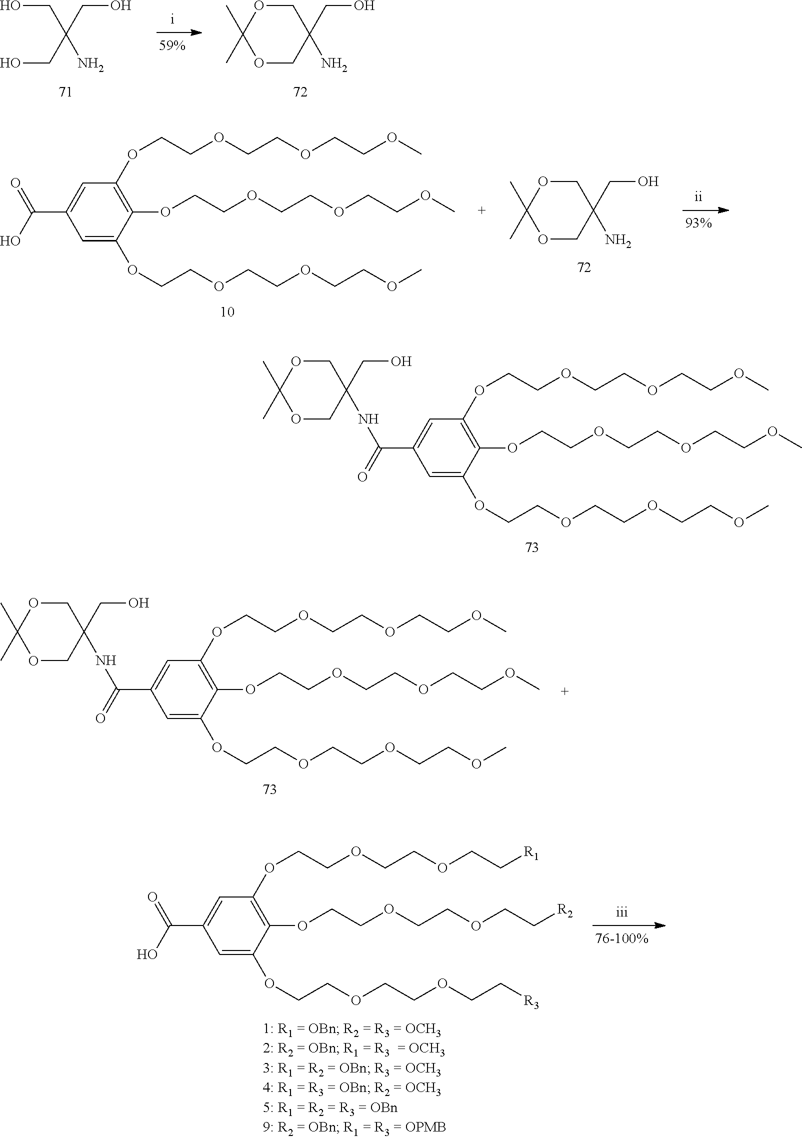

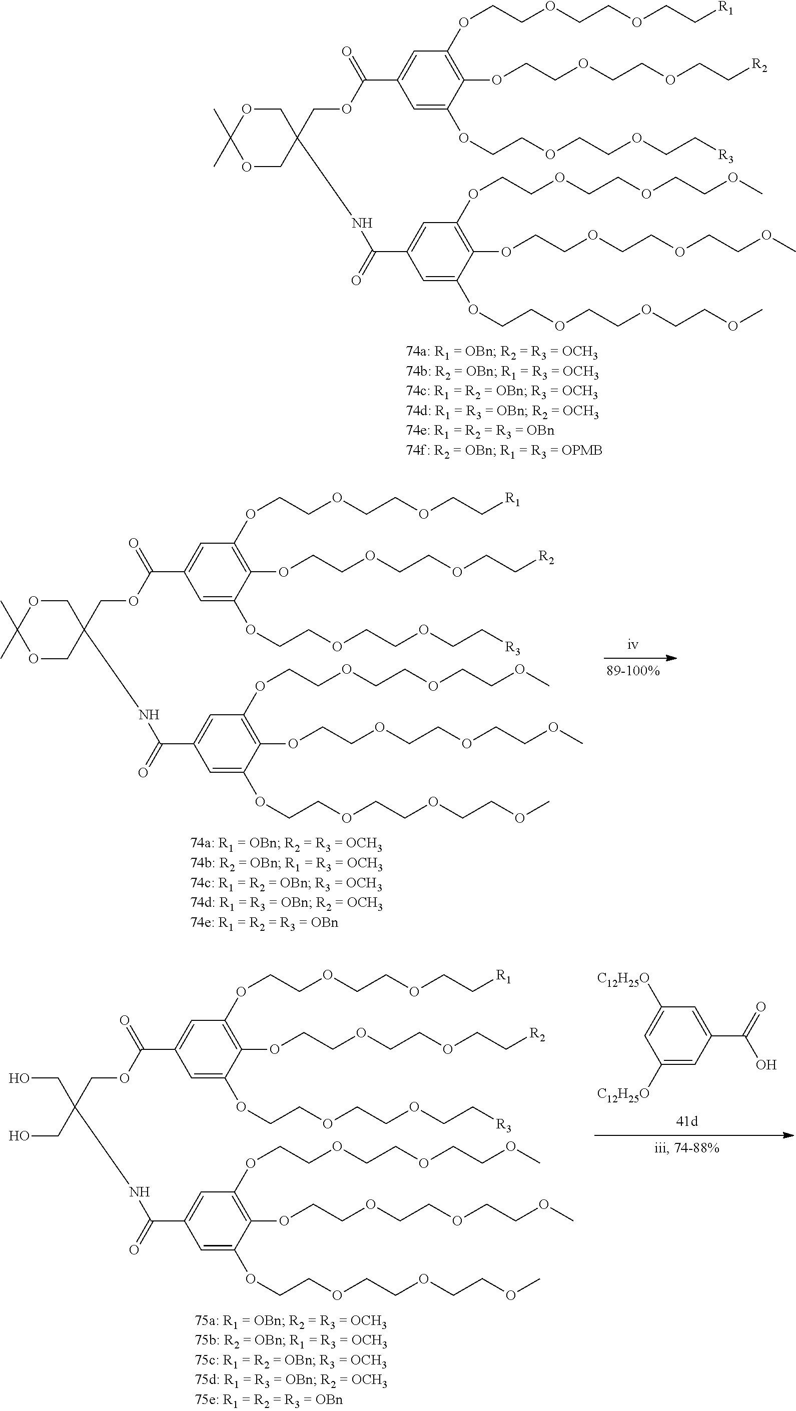

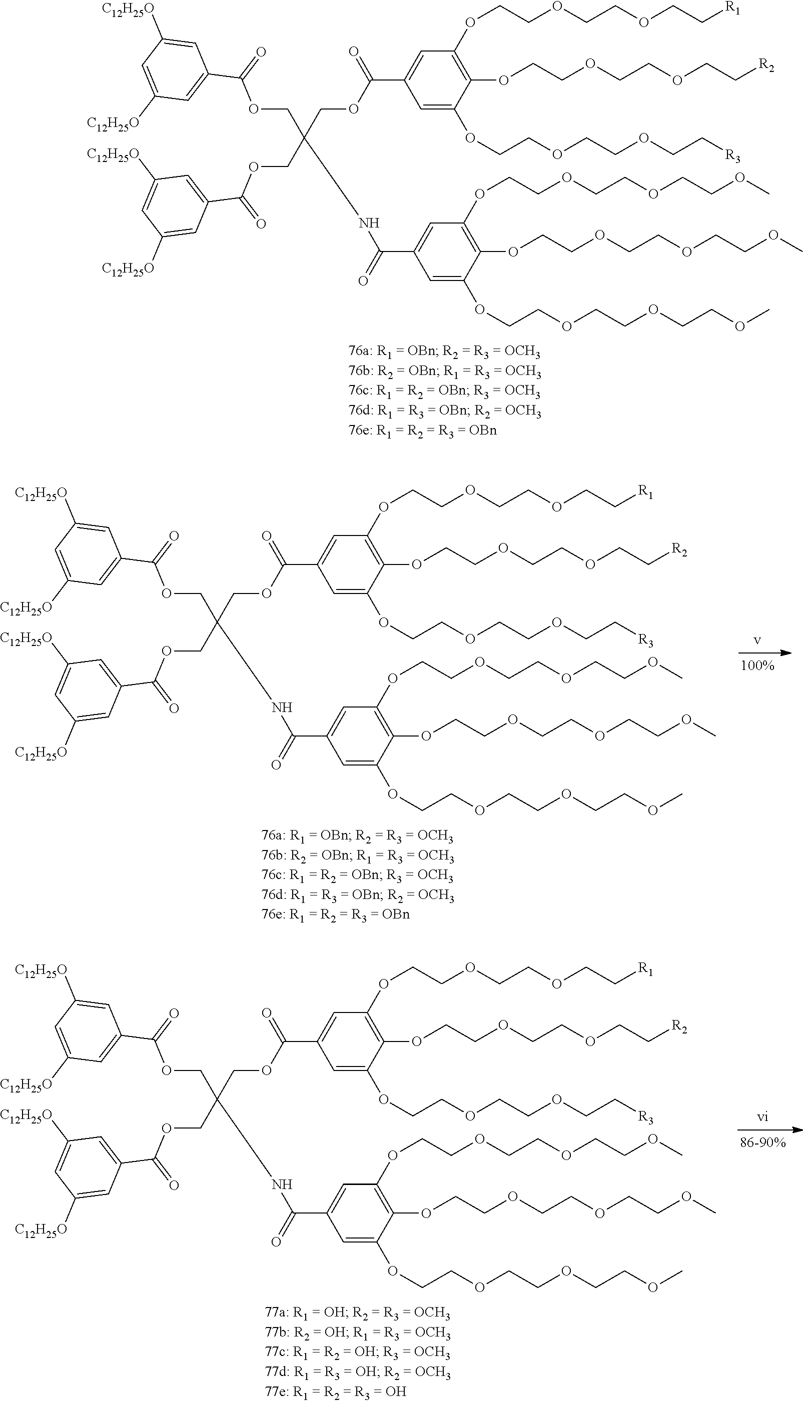

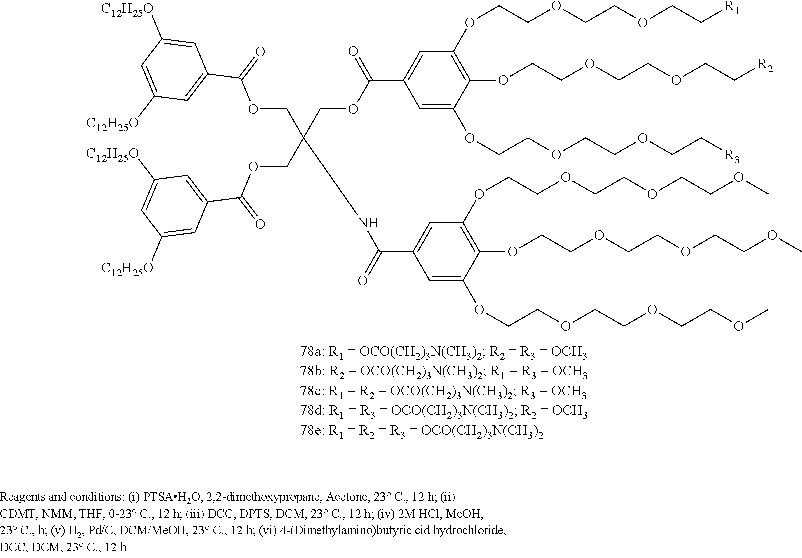

Compounds 72 and 73 were synthesized according to literature procedures (Zhang, et al., J. Am. Chem. Soc. 2015, 137, 13334-13344; Xiao, et al., Proc. Natl. Acad. Sci. U.S.A. 2020, 117, 11931-11939).

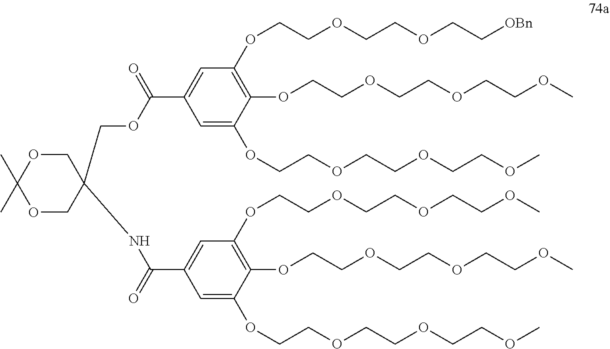

Compound 74a. To a DCM (5 mL) solution of compound 1 (230 mg, 0.33 mmol), compound 73 (250 mg, 0.33 mmol) and DPTS (100 mg, 0.33 mmol), was added DCC (140 mg, 0.66 mmol). The mixture was allowed to stir at 23° C. for 12 h. The precipitate was filtered, and the filtrate was concentrated to dryness. The crude product was further purified by column chromatography with a mobile phase of DCM/MeOH=20/1 to yield the title compound as a colorless oil (420 mg, 89%).

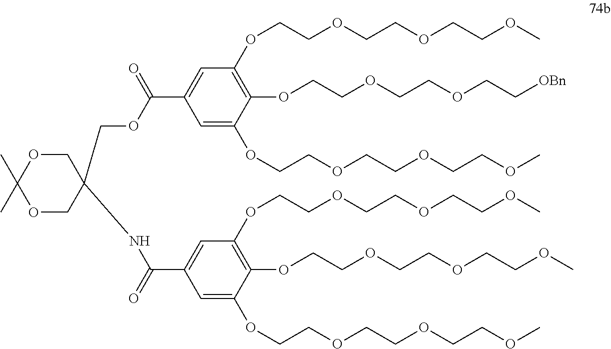

Compound 74b. To a DCM (10 mL) solution of compound 2 (229 mg, 0.304 mmol), compound 73 (229 mg, 0.334 mmol) and DPTS (134 mg, 0.456 mmol), was added DCC (188 mg, 0.912 mmol). The mixture was allowed to stir at 23° C. for 12 h. The precipitate was filtered, and the filtrate was concentrated to dryness. The crude product was further purified by column chromatography with a mobile phase of DCM/MeOH=20/1 to yield the title compound as a colorless oil (430 mg, 100%).

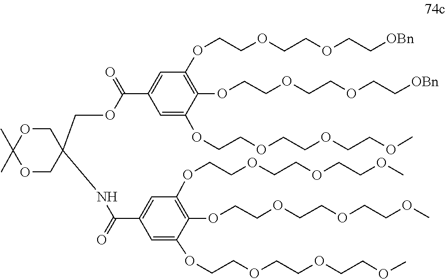

Compound 74c. To a DCM (5 mL) solution of compound 3 (250 mg, 0.334 mmol), compound 73 (250 mg, 0.334 mmol) and DPTS (102 mg, 0.334 mmol), was added DCC (142 mg, 0.668 mmol). The mixture was allowed to stir at 23° C. for 12 h. The precipitate was filtered, and the filtrate was concentrated to dryness. The crude product was further purified by column chromatography with a mobile phase of DCM/MeOH=60/1 to 20/1 to yield the title compound as a colorless oil (376 mg, 76%).

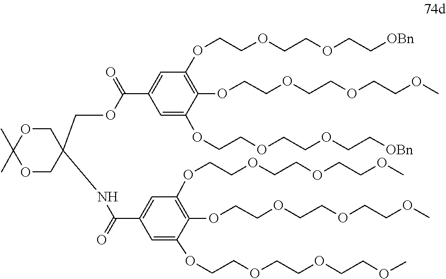

Compound 74d. To a DCM (5 mL) solution of compound 4 (0.25 mg, 0.33 mmol, 1 equiv), compound 73 (0.25 g, 0.33 mmol, 1 equiv) and DPTS (0.10 g, 0.33 mmol, 1 equiv), was added DCC (0.14 g, 0.66 mmol, 2 equiv). The mixture was allowed to stir at 23° C. for 12 h. The precipitate was filtered, and the filtrate was concentrated to dryness. The crude product was further purified by column chromatography with a mobile phase of DCM/MeOH=30/1 to yield the title compound as a colorless oil (0.43 g, 88%).

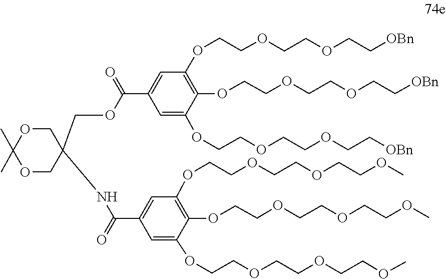

Compound 74e. To a DCM (5 mL) solution of compound 5 (280 mg, 0.330 mmol), compound 73 (250 mg, 0.330 mmol) and DPTS (103 mg, 0.330 mmol), was added DCC (140 mg, 0.660 mmol). The mixture was allowed to stir at 23° C. for 12 h. The precipitate was filtered, and the filtrate was concentrated to dryness. The crude product was further purified by column chromatography with a mobile phase of DCM/MeOH=20/1 to yield the title compound as a colorless oil (440 mg, 85%).

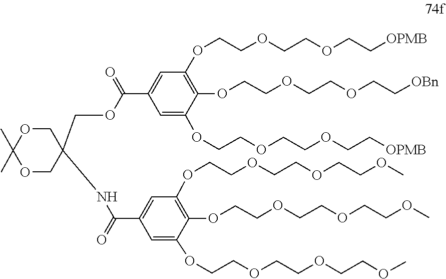

Compound 74f. To a DCM (10 mL) solution of compound 9 (558 mg, 0.62 mmol), compound 73 (425 mg, 0.57 mmol) and DPTS (252 mg, 0.86 mmol), was added DCC (352 mg, 1.71 mmol). The mixture was allowed to stir at 23° C. for 12 h. The precipitate was filtered, and the filtrate was concentrated to dryness. The crude product was further purified by column chromatography with a mobile phase of DCM/MeOH=20/1 to yield the title compound as a colorless oil (814 mg, 88%).

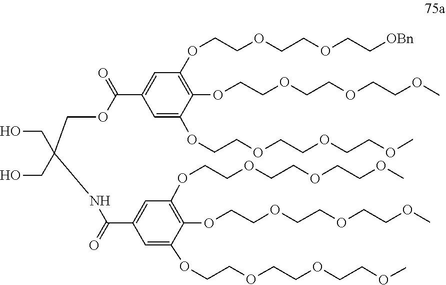 [SMILES: COCCOCCOCCOc1cc(C(=O)NC(CO)(CO)COC(=O)c2cc(OCCOCCOCCOC)c(OCCOCCOCCOC)c(OCCOCCOCCOCc3ccccc3)c2)cc(OCCOCCOCCOC)c1OCCOCCOCCOC]

Compound 75a. To a MeOH solution (10 mL) of compound 74a (400 mg, 0.63 mmol) was added 3 mL of HCl aqueous solution (2 M). The mixture was allowed to stir at 23° C. for 2h and saturated NaHCO₃solution (20 mL) was added. The mixture was extracted with DCM (50 mL) for 3 times. An organic extract was dried over MgSO₄ and evaporated to dryness under reduced pressure to yield the title compound as a colorless viscous liquid without further purification (366 mg, 95%).

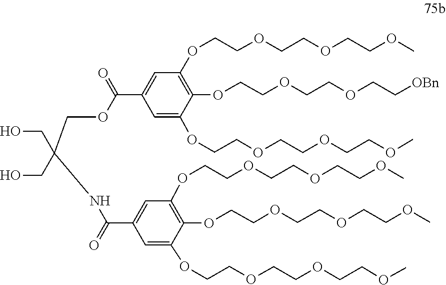 [SMILES: COCCOCCOCCOc1cc(C(=O)NC(CO)(CO)COC(=O)c2cc(OCCOCCOCCOC)c(OCCOCCOCCOCc3ccccc3)c(OCCOCCOCCOC)c2)cc(OCCOCCOCCOC)c1OCCOCCOCCOC]

Compound 75b. Compound 75b was synthesized from compound 74b (430 mg, 0.334 mmol) following a procedure similar to that used for the synthesis of compound 75a. The title compound was obtained as a colorless viscous liquid without further purification (410 mg, 89%).

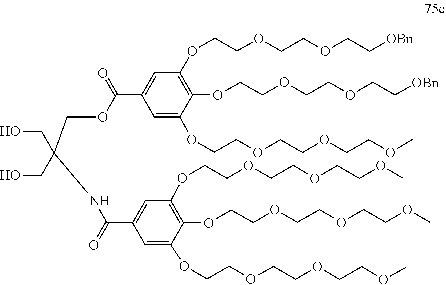 [SMILES: COCCOCCOCCOc1cc(C(=O)NC(CO)(CO)COC(=O)c2cc(OCCOCCOCCOC)c(OCCOCCOCCOCc3ccccc3)c(OCCOCCOCCOCc3ccccc3)c2)cc(OCCOCCOCCOC)c1OCCOCCOCCOC]

Compound 75c. Compound 75c was synthesized from compound 74c (350 mg, 0.234 mmol) following a procedure similar to that used for the synthesis of compound 75a. The title compound was obtained as a colorless viscous liquid without further purification (310 mg, 91

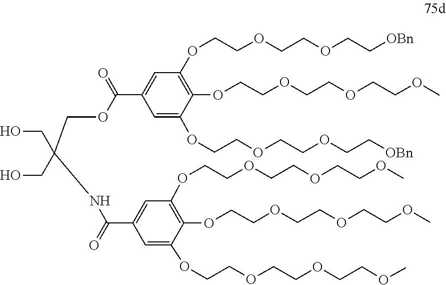 [SMILES: COCCOCCOCCOc1cc(C(=O)NC(CO)(CO)COC(=O)c2cc(OCCOCCOCCOCc3ccccc3)c(OCCOCCOCCOC)c(OCCOCCOCCOCc3ccccc3)c2)cc(OCCOCCOCCOC)c1OCCOCCOCCOC]

Compound 75d. Compound 75d was synthesized from compound 74d (0.36 g, 0.24 mmol) following a procedure similar to that used for the synthesis of compound 75a. The title compound was obtained as a a colorless oil without further purification (0.35 g, 100%).

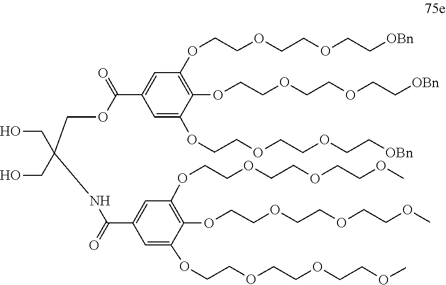 [SMILES: COCCOCCOCCOc1cc(C(=O)NC(CO)(CO)COC(=O)c2cc(OCCOCCOCCOCc3ccccc3)c(OCCOCCOCCOCc3ccccc3)c(OCCOCCOCCOCc3ccccc3)c2)cc(OCCOCCOCCOC)c1OCCOCCOCCOC]

Compound 75e. Compound 75e was synthesized from compound 74e (330 mg, 0.210 mmol) following a procedure similar to that used for the synthesis of compound 75a. The title compound was obtained as a colorless viscous liquid without further purification (320 mg, 100%).

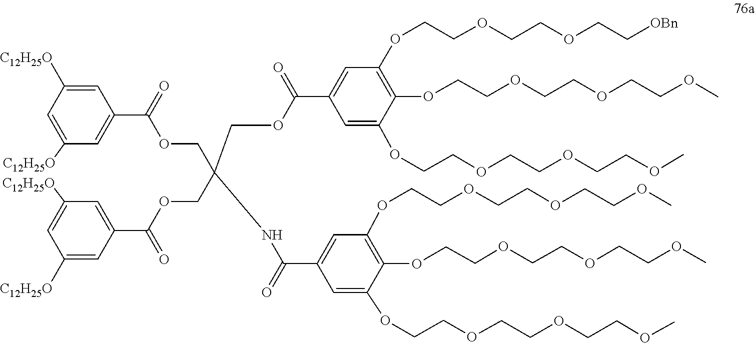

Compound 76a. To a DCM (5 mL) solution of compound 41d (307 mg, 0.624 mmol), compound 75a (410 mg, 0.297 mmol) and DPTS (219 mg, 0.742 mmol), was added DCC (306 mg, 1.49 mmol). The mixture was allowed to stir at 23° C. for 12 h. The precipitate was filtered, and the filtrate was concentrated to dryness. The crude product was further purified by column chromatography with a mobile phase of DCM/MeOH=20/1 to yield the title compound as a colorless oil (586 mg, 85%).

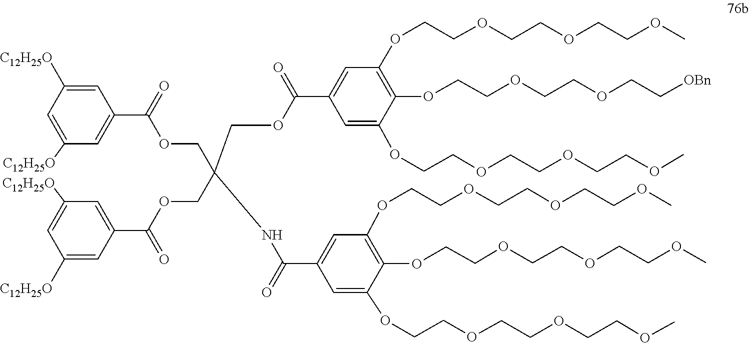

Compound 76b. To a DCM (10 mL) solution of compound 41d (307 mg, 0.624 mmol), compound 75b (410 mg, 0.297 mmol) and DPTS (219 mg, 0.742 mmol), was added DCC (306 mg, 1.49 mmol). The mixture was allowed to stir at 23° C. for 12 h. The precipitate was filtered, and the filtrate was concentrated to dryness. The crude product was further purified by column chromatography with a mobile phase of DCM/MeOH=20/1 to yield the title compound as a colorless oil (607 mg, 88%).

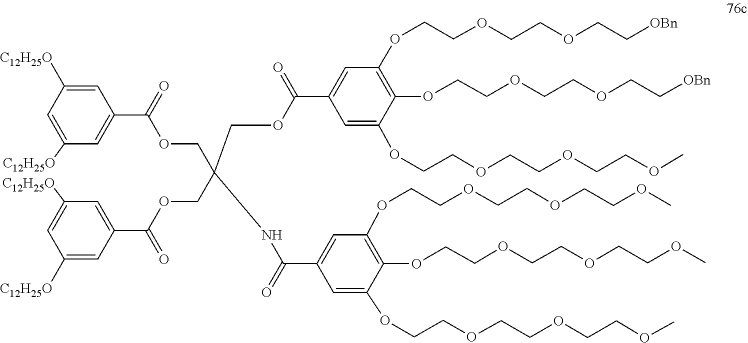

Compound 76c. To a DCM (5 mL) solution of compound 41d (200 mg, 0.400 mmol), compound 75c (280 mg, 0.197 mmol) and DPTS (119 mg, 0.380 mmol), was added DCC (118 mg, 0.571 mmol). The mixture was allowed to stir at 23° C. for 12 h. The precipitate was filtered, and the filtrate was concentrated to dryness. The crude product was further purified by column chromatography with a mobile phase of DCM/MeOH=20/1 to yield the title compound as a colorless oil (375 mg, 83%).

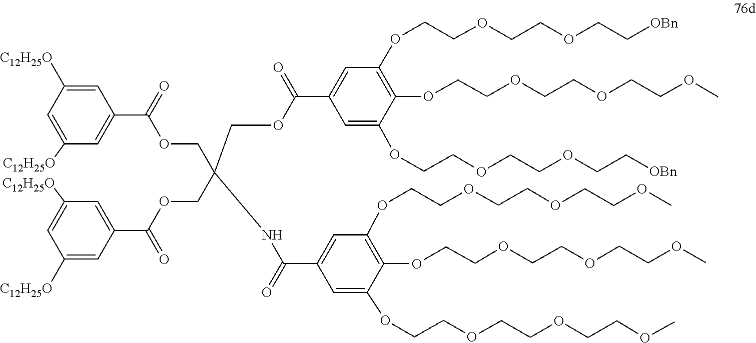

Compound 76d. To a DCM (10 mL) solution of compound 41d (0.19 g, 0.38 mmol, 2.1 equiv), compound 75d (0.26 g, 0.18 mmol, 1 equiv) and DPTS (0.11 g, 0.36 mmol, 2 equiv), was added DCC (0.11 g, 0.54 mmol, 3 equiv). The mixture was allowed to stir at 23° C. for 12 h. The precipitate was filtered, and the filtrate was concentrated to dryness. The crude product was further purified by column chromatography with a mobile phase of DCM/MeOH=30/1 to yield the title compound as a colorless oil (0.32 g, 74%).

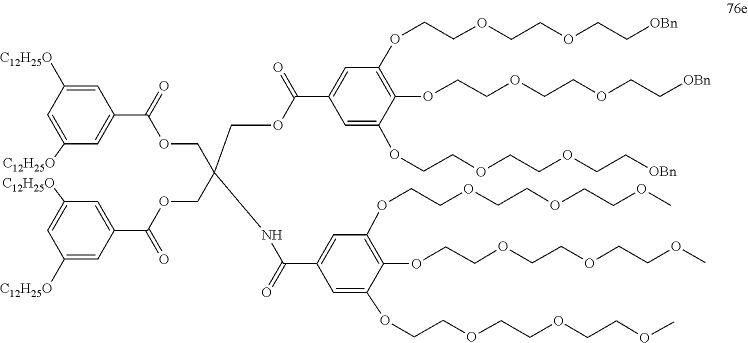

Compound 76e. To a DCM (10 mL) solution of compound 41d (220 mg, 0.44 mmol), compound 75e (320 mg, 0.21 mmol) and DPTS (130 mg, 0.42 mmol), was added DCC (130 mg, 0.63 mmol). The mixture was allowed to stir at 23° C. for 12 h. The precipitate was filtered, and the filtrate was concentrated to dryness. The crude product was further purified by column chromatography with a mobile phase of DCM/MeOH=30/1 to yield the title compound as a colorless oil (440 mg, 85%).

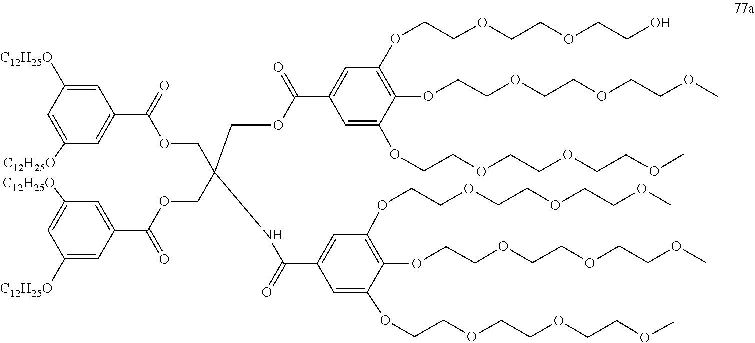

Compound 77a. To a mixed DCM (16 mL) and MeOH (8 mL) solution of compound 76a (250 mg, 0.112 mmol) was added Pd/C (25 mg, 10 wt %). The mixture was bubbling with H₂ for 30 min and then allowed to stir at 23° C. for 12 h under H₂ atmosphere. The mixture was then filtered through Celite®. The filtrate was concentrated to dryness and without further purification to yield the title compound as a colorless oil (240 mg, 100%).

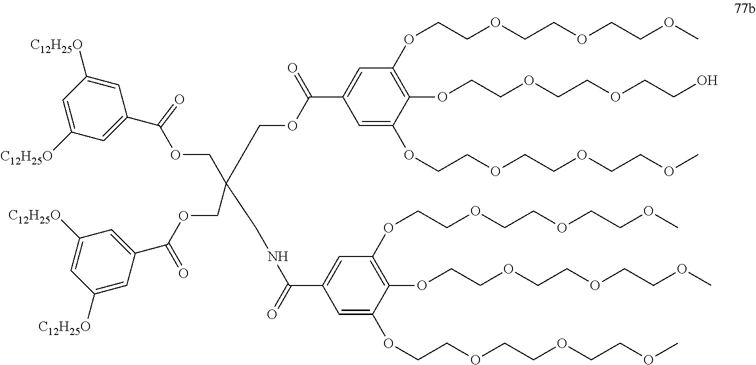

Compound 77b. Compound 77b was synthesized from compound 76b (607 mg, 0.261 mmol) following a procedure similar to that used for the synthesis of compound 77a. The title compound was obtained as a colorless oil (582 mg, 100%).

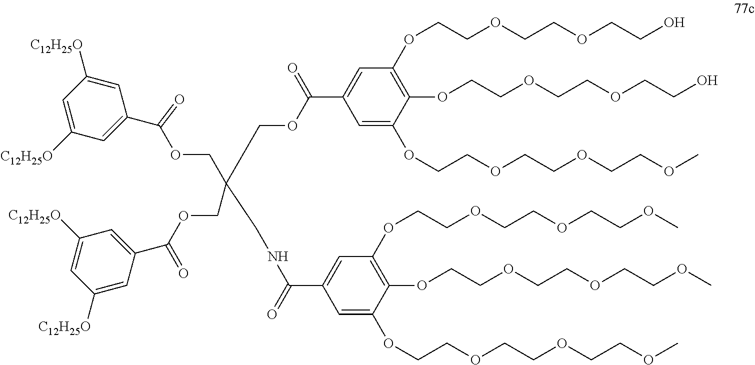

Compound 77c. Compound 77c was synthesized from compound 76c (360 mg, 0.150 mmol) following a procedure similar to that used for the synthesis of compound 77a. The title compound was obtained as a colorless oil (330 mg, 100%).

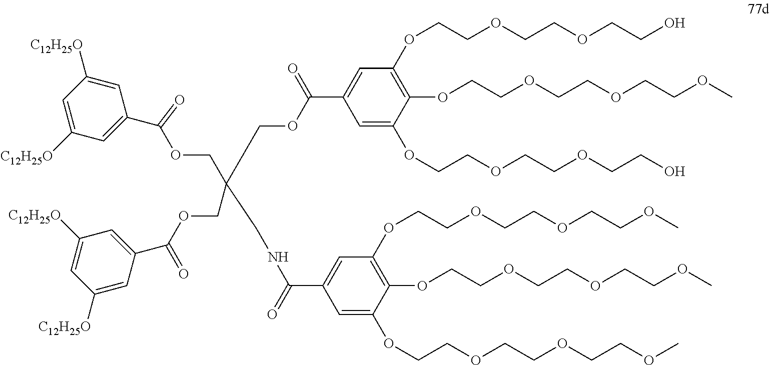

Compound 77d. Compound 77d was synthesized from compound 76d (0.23 g, 0.096 mmol) following a procedure similar to that used for the synthesis of compound 77a. The title compound was obtained as a colorless oil (0.21 g, 100%).

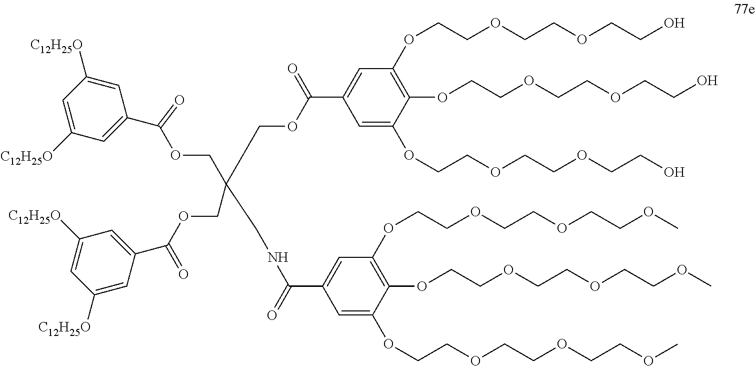

Compound 77e. Compound 77e was synthesized from compound 76e (300 mg, 0.121 mmol) following a procedure similar to that used for the synthesis of compound 77a. The title compound was obtained as a colorless oil (266 mg, 100%).

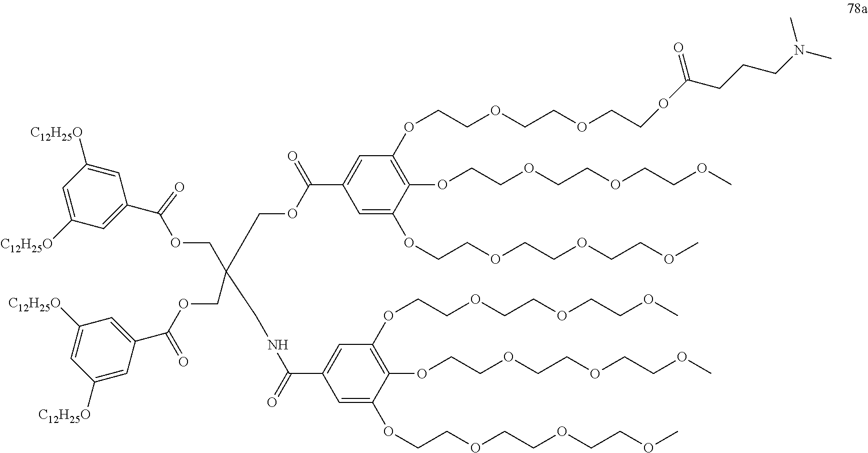

Compound 78a (6/1_{DMBA}¹). Compound 77a (560 mg, 0.25 mmol, 1 equiv) and 4-(dimethylamino)butyric acid hydrochloride (50 mg, 0.30 mmol, 1.2 equiv) were dissolved in 6 mL dry DCM. DCC (155 mg, 0.75 mmol, 3 equiv) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=10/1, 6/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (504 mg, 86%).

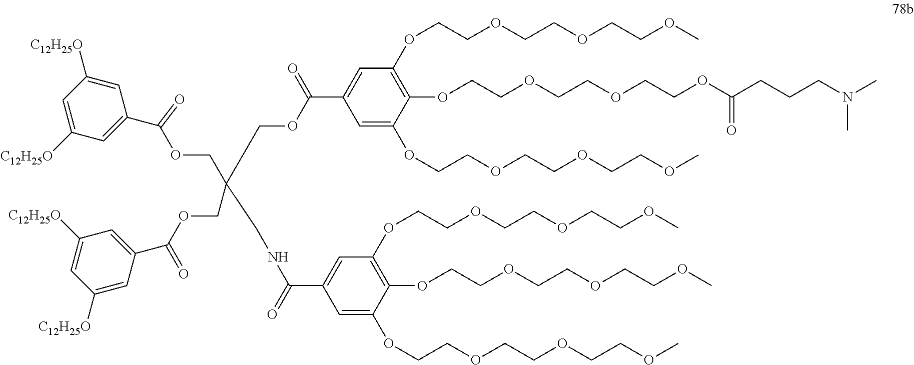

Compound 78b (6/1_{DMBA}²). Compound 78b was synthesized from compound 77b (311 mg, 0.139 mmol), 4-(dimethylamino)butyric acid hydrochloride (28 mg, 0.167 mmol) and DCC (57 mg, 0.278 mmol) following a procedure similar to that used for the synthesis of compound 78a. The title compound was obtained as a light-yellow oil (290 mg, 90%).

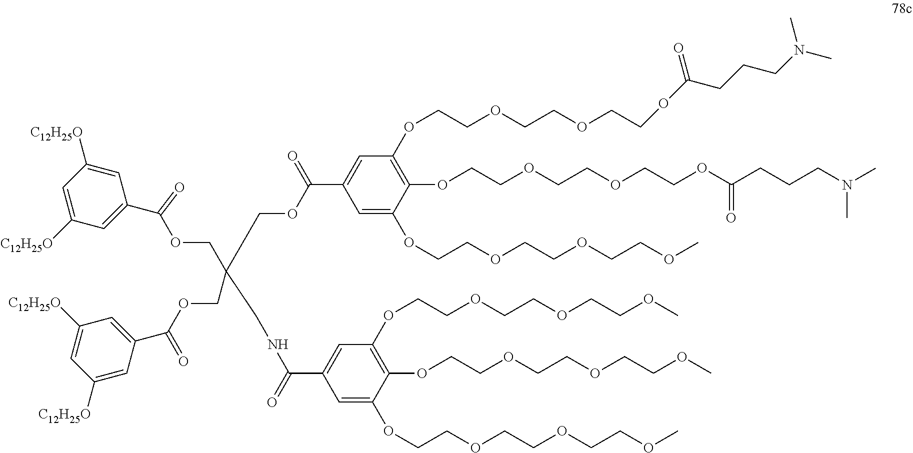

Compound 78c (6/2_{DMBA}^{1,2}). Compound 78c was synthesized from compound 77c (320 mg, 0.144 mmol), 4-(dimethylamino)butyric acid hydrochloride (70 mg, 0.432 mmol) and DCC (119 mg, 0.576 mmol) following a procedure similar to that used for the synthesis of compound 78a. The title compound was obtained as a light-yellow oil (316 mg, 90%).

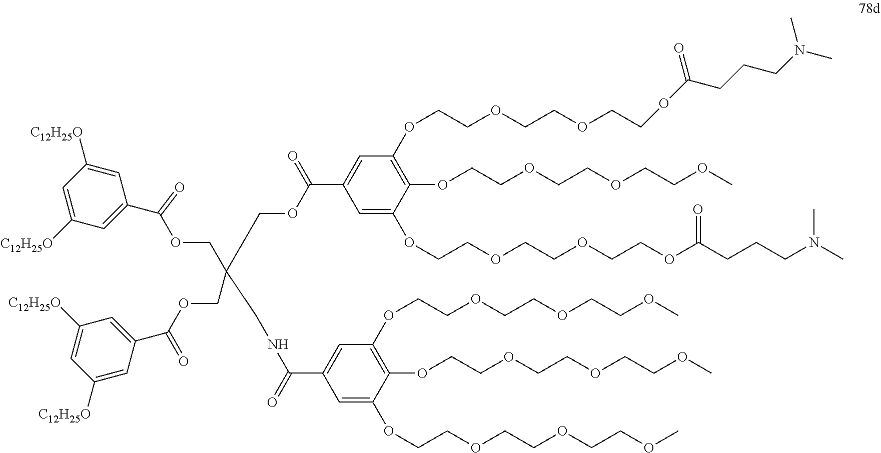

Compound 78d (6/2_{DMBA}^{1,3}). Compound 78d was synthesized from compound 77d (0.17 g, 0.077 mmol), 4-(dimethylamino)butyric acid hydrochloride (28 mg, 0.170 mmol) and DCC (48 mg, 0.231 mmol) following a procedure similar to that used for the synthesis of compound 78a. The title compound was obtained as a light-yellow oil (0.17 g, 90%).

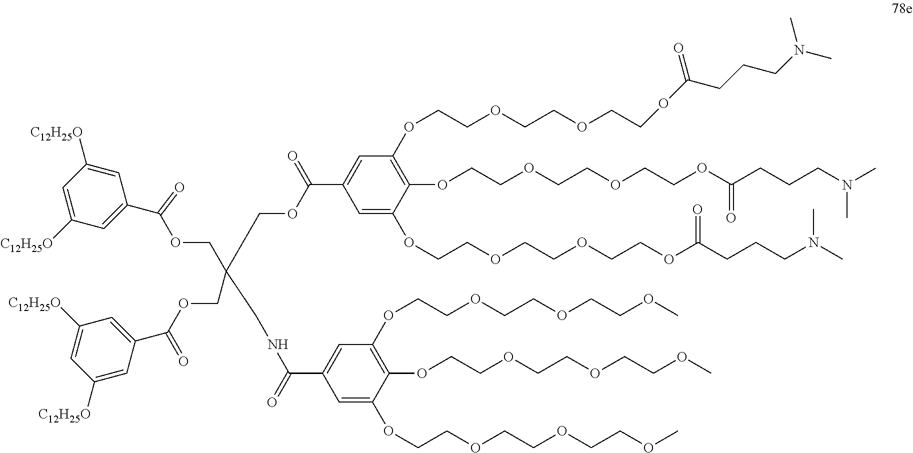

Compound 78e (6/3_{DMBA}^{1,3}). Compound 78e was synthesized from compound 77e (260 mg, 0.116 mmol), 4-(dimethylamino)butyric acid hydrochloride (78 mg, 0.467 mmol) and DCC (95 mg, 0.467 mmol) following a procedure similar to that used for the synthesis of compound 78a. The title compound was obtained as a light-yellow oil (290 mg, 90%). 3.8 Synthesis of Hybrid Twin-Mix IAJDs 26-29, 47, 49-52 in Library 6

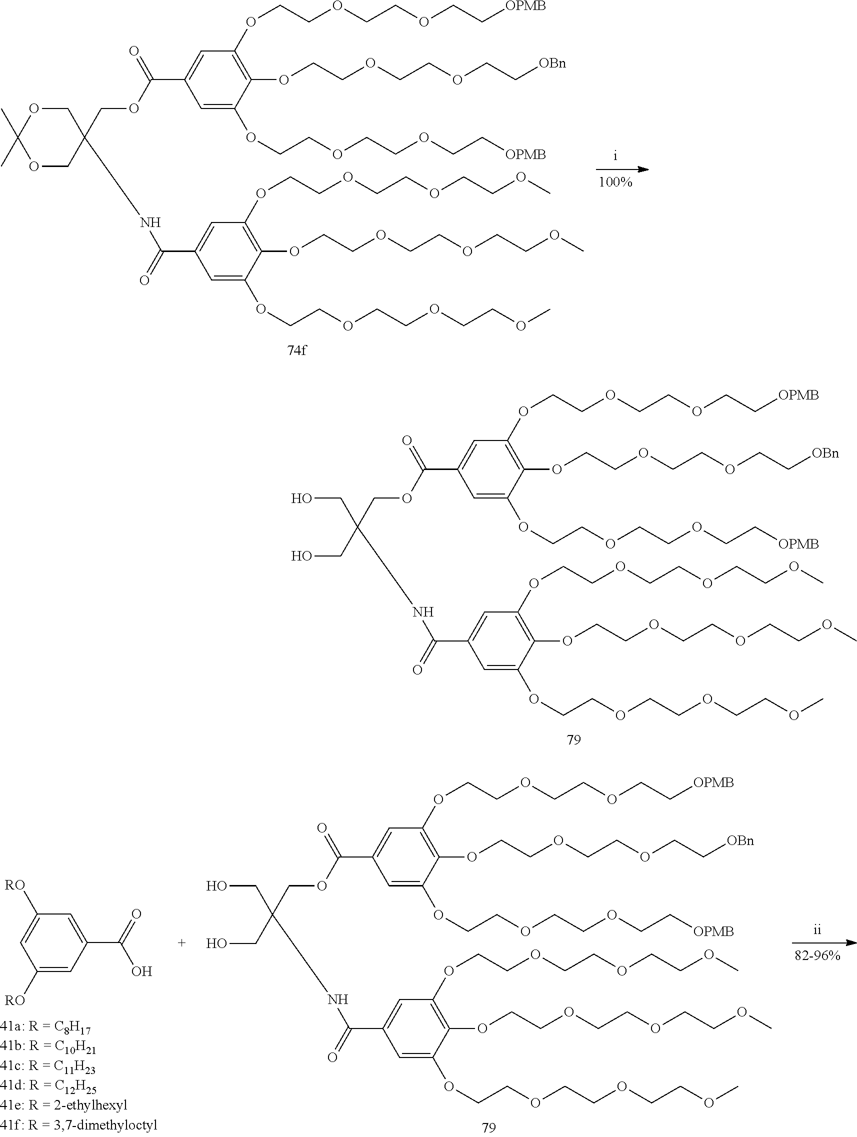

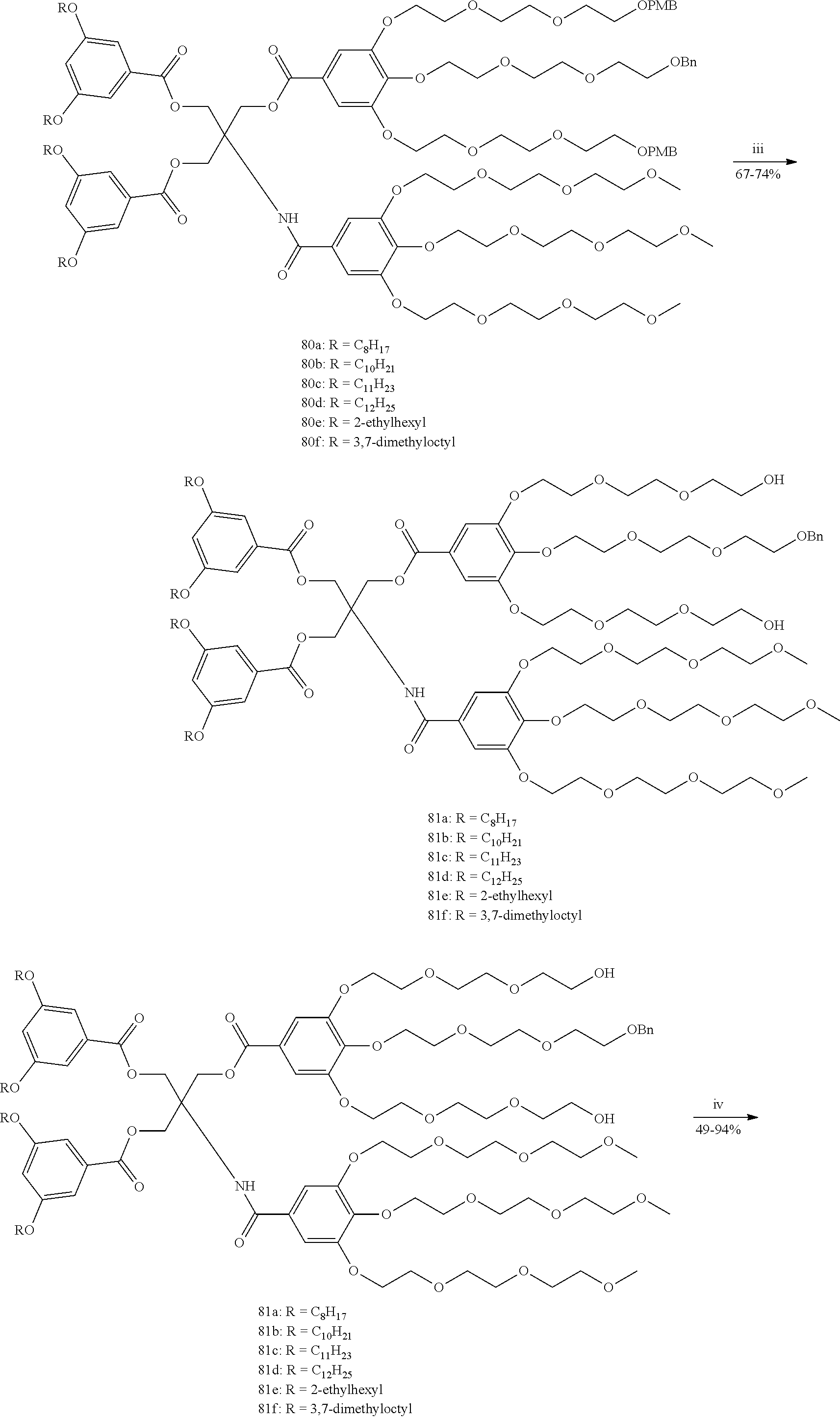

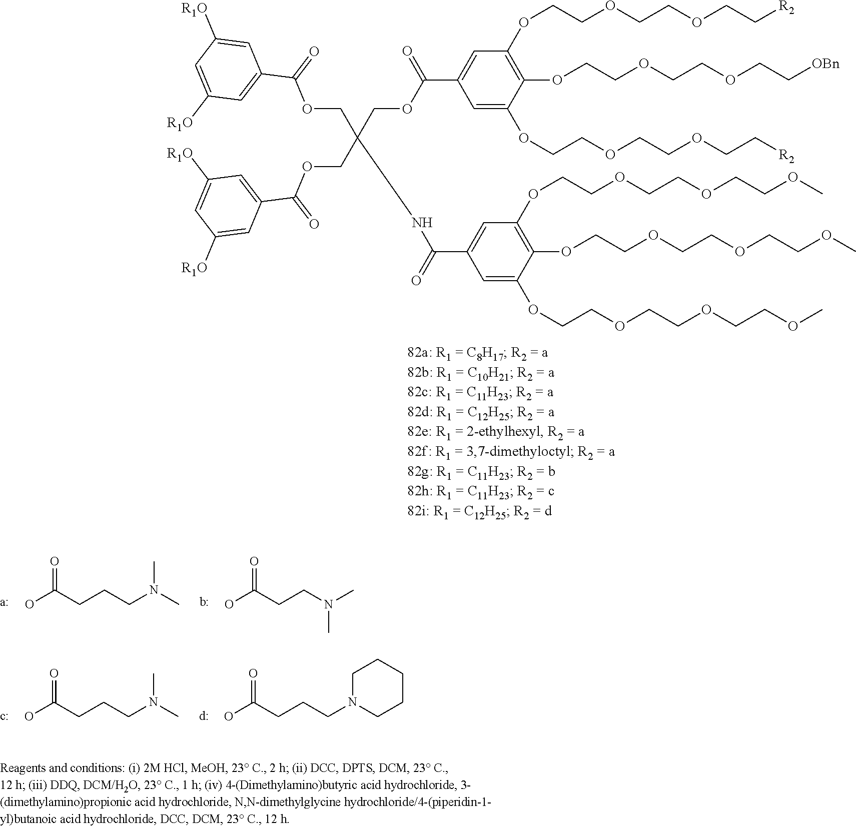

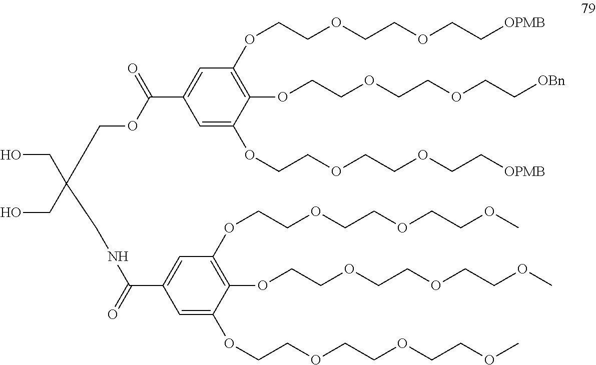

Compound 79. To a MeOH solution (10 mL) of compound 74f (800 mg, 0.489 mmol) was added 3 mL of HCl aqueous solution (2 M). The mixture was allowed to stir at 23° C. for 2 h and saturated NaHCO₃solution (20 mL) was added. The mixture was extracted with DCM (50 mL) for 3 times. An organic extract was dried over anhydrous MgSO₄ and evaporated to dryness under reduced pressure to yield the title compound as a colorless viscous liquid without further purification (778 mg, 100%).

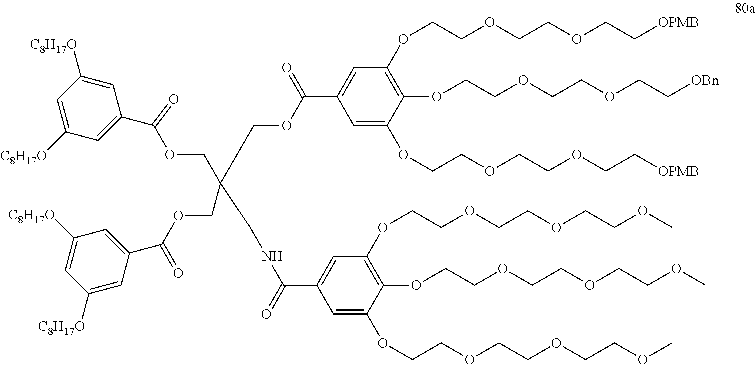

Compound 80a. To a DCM (10 mL) solution of compound 41a (79 mg, 0.21 mmol), compound 79 (160 mg, 0.10 mmol) and DPTS (62 mg, 0.20 mmol), was added DCC (62 mg, 0.30 mmol). The mixture was allowed to stir at 23° C. for 12 h. The precipitate was filtered, and the filtrate was concentrated to dryness. The crude product was further purified by column chromatography with a mobile phase of DCM/MeOH=20/1 to yield the title compound as a colorless oil (190 mg, 83%).

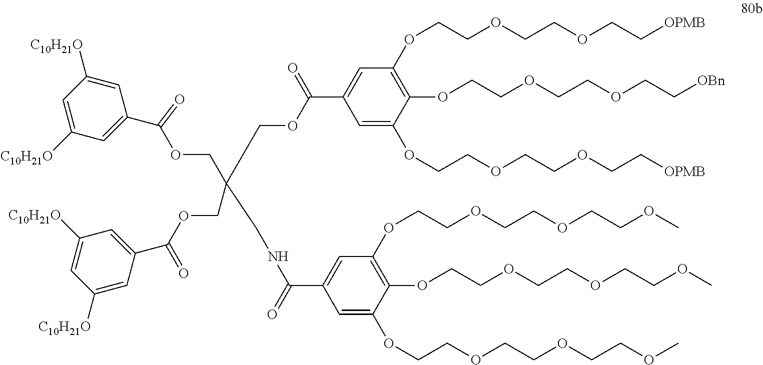

Compound 80b. Compound 80b was synthesized from compound 41b (174 mg, 0.401 mmol), compound 79 (290 mg, 0.182 mmol), DPTS (134 mg, 0.455 mmol) and DCC (188 mg, 0.910 mmol) following a procedure similar to that used for the synthesis of compound 80a. The title compound was obtained as a colorless oil (423 mg, 96%).

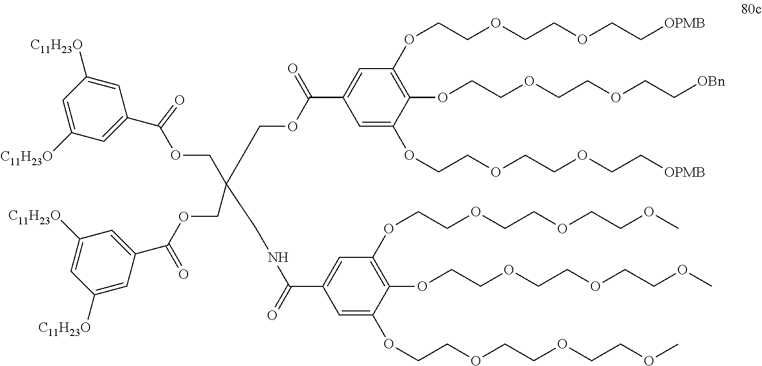

Compound 80c. Compound 80c was synthesized from compound 41c (150 mg, 0.345 mmol), compound 79 (249 mg, 0.157 mmol), DPTS (116 mg, 0.392 mmol) and DCC (161 mg, 0.785 mmol) following a procedure similar to that used for the synthesis of compound 80a. The title compound was obtained as a colorless oil (332 mg, 87%).

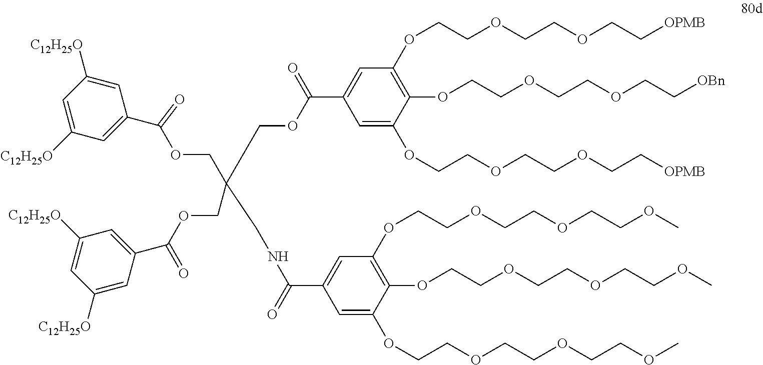

Compound 80d. Compound 80d was synthesized from compound 41d (170 mg, 0.347 mmol), compound 79 (251 mg, 0.158 mmol), DPTS (116 mg, 0.371 mmol) and DCC (163 mg, 0.789 mmol) following a procedure similar to that used for the synthesis of compound 80a. The title compound was obtained as a colorless oil (355 mg, 89%).

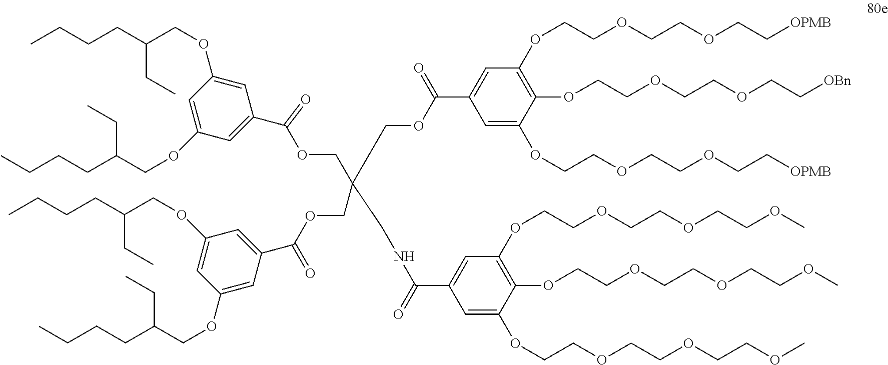

Compound 80e. Compound 80e was synthesized from compound 41e (79 mg, 0.21 mmol), compound 79 (160 mg, 0.10 mmol), DPTS (62 mg, 0.20 mmol) and DCC (62 mg, 0.30 mmol) following a procedure similar to that used for the synthesis of compound 80a. The title compound was obtained as a colorless oil (194 mg, 84%).

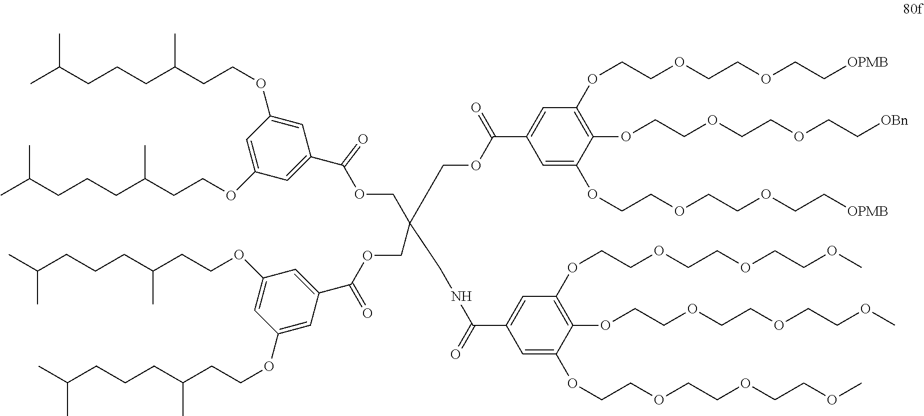

Compound 80f. Compound 80f was synthesized from compound 41f (150 mg, 0.345 mmol), compound 79 (249 mg, 0.157 mmol), DPTS (116 mg, 0.392 mmol) and DCC (161 mg, 0.785 mmol) following a procedure similar to that used for the synthesis of compound 80a. The title compound was obtained as a colorless oil (314 mg, 82%).

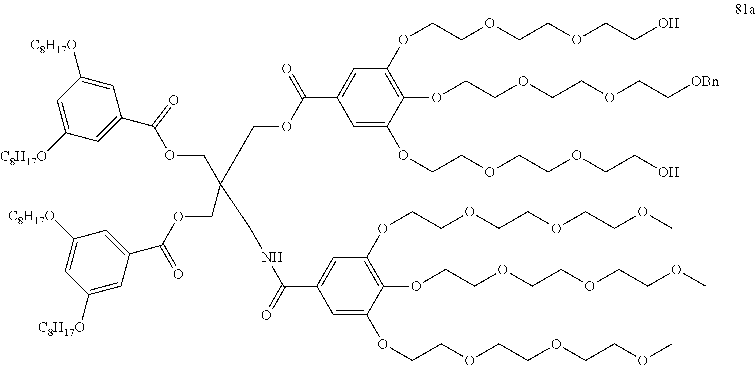

Compound 81a. To a mixed solution of DCM (5 mL) and water (0.25 mL) of compound 80a (180 mg, 0.078 mmol), was added DDQ (44 mg, 0.195 mmol). The mixture was allowed to stir at 23° C. for 1 h. The precipitates were filtered out and DCM (20 mL) was added. The mixture was washed by NaHCO₃aqueous solution (saturated), NaHSO₃ aqueous solution (2%) and NaHCO₃aqueous solution (saturated) successively. The organic phase was dried over anhydrous MgSO₄ and filtered. The filtrate was concentrated and purified by column chromatography with a mobile phase of EtOAc/MeOH=10/1 to yield the title compound as a light-yellow oil (112 mg, 67%).

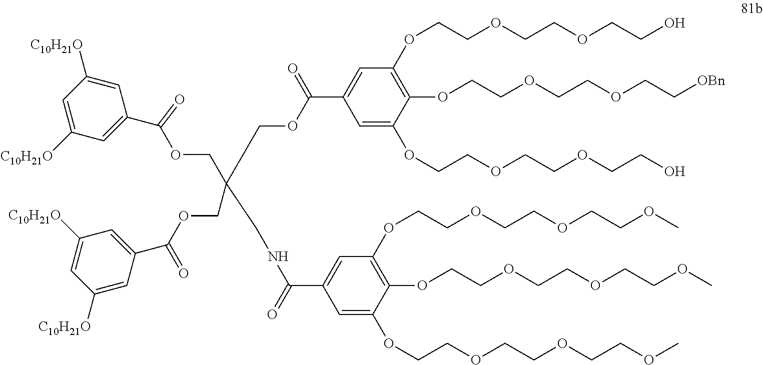

Compound 81b. Compound 81b was synthesized from compound 80b (420 mg, 0.173 mmol) following a procedure similar to that used for the synthesis of compound 81a. The title compound was obtained as a colorless oil (278 mg, 73%).

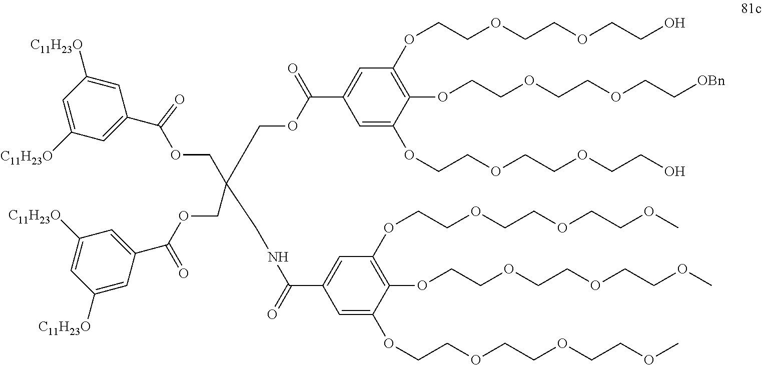

Compound 81c. Compound 81c was synthesized from compound 80c (350 mg, 0.141 mmol) following a procedure similar to that used for the synthesis of compound 81a. The title compound was obtained as a colorless oil (229 mg, 73%).

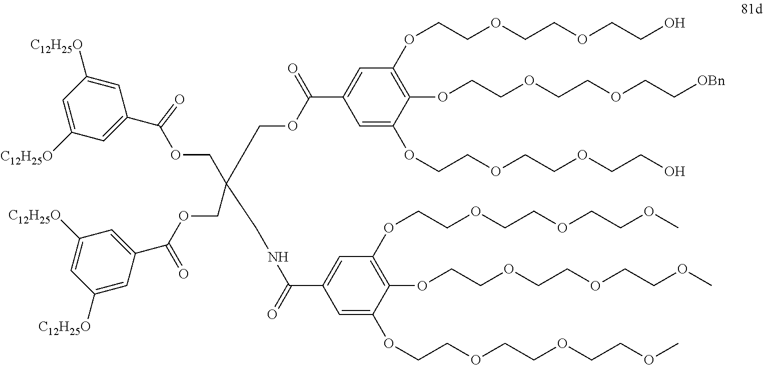

Compound 81d. Compound 81d was synthesized from compound 80d (348 mg, 0.137 mmol) following a procedure similar to that used for the synthesis of compound 81a. The title compound was obtained as a colorless oil (249 mg, 72%).

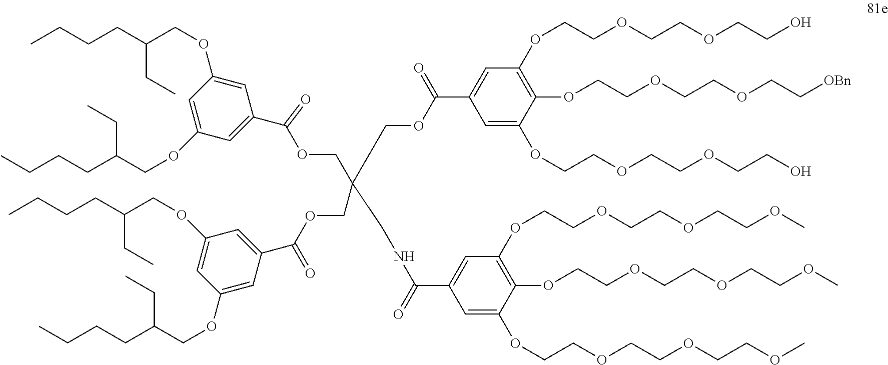 [SMILES: CCCCC(CC)COc1cc(OCC(CC)CCCC)cc(C(=O)OCC(CCOC(=O)c2cc(OCCOCCOCCO)c(OCCOCCOCCOCc3ccccc3)c(OCCOCCOCCO)c2)(CNC(=O)c2cc(OCCOCCOCCOC)c(OCCOCCOCCOC)c(OCCOCCOCCOC)c2)COC(=O)c2cc(OCC(CC)CCCC)cc(OCC(CC)CCCC)c2)c1]

Compound 81e. Compound 81e was synthesized from compound 80e (190 mg, 0.082 mmol) following a procedure similar to that used for the synthesis of compound 81a. The title compound was obtained as a light-yellow oil (130 mg, 74%).

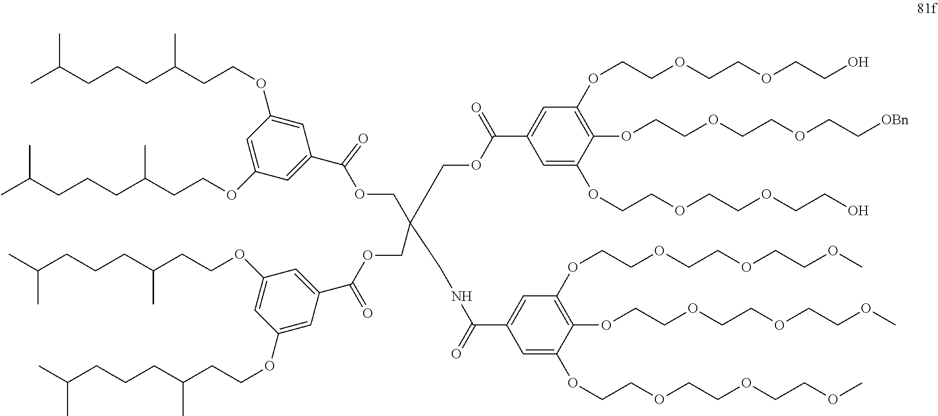 [SMILES: COCCOCCOCCOc1cc(C(=O)NCC(CCOC(=O)c2cc(OCCOCCOCCO)c(OCCOCCOCCOCc3ccccc3)c(OCCOCCOCCO)c2)(COC(=O)c2cc(OCCC(C)CCCC(C)C)cc(OCCC(C)CCCC(C)C)c2)COC(=O)c2cc(OCCC(C)CCCC(C)C)cc(OCCC(C)CCCC(C)C)c2)cc(OCCOCCOCCOC)c1OCCOCCOCCOC]

Compound 81f. Compound 81f was synthesized from compound 80f (330 mg, 0.136 mmol) following a procedure similar to that used for the synthesis of compound 81a. The title compound was obtained as a colorless oil (215 mg, 72%).

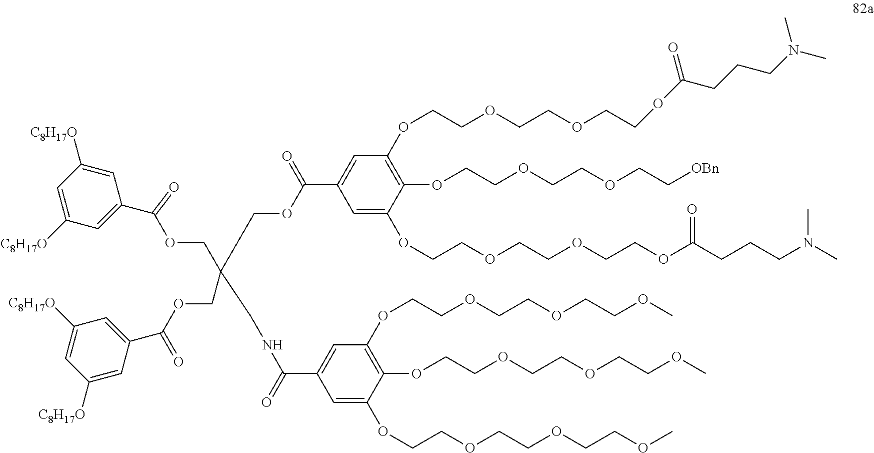

Compound 82a (6/2_{DMBA}^{1,3}_{Bn}²-C8). Compound 81a (110 mg, 0.053 mmol) and 4-(dimethylamino)butyric acid hydrochloride (20 mg, 0.117 mmol) were dissolved in 5 mL dry DCM. DCC (33 mg, 0.159 mmol) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=10/1, 6/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (110 mg, 92%).

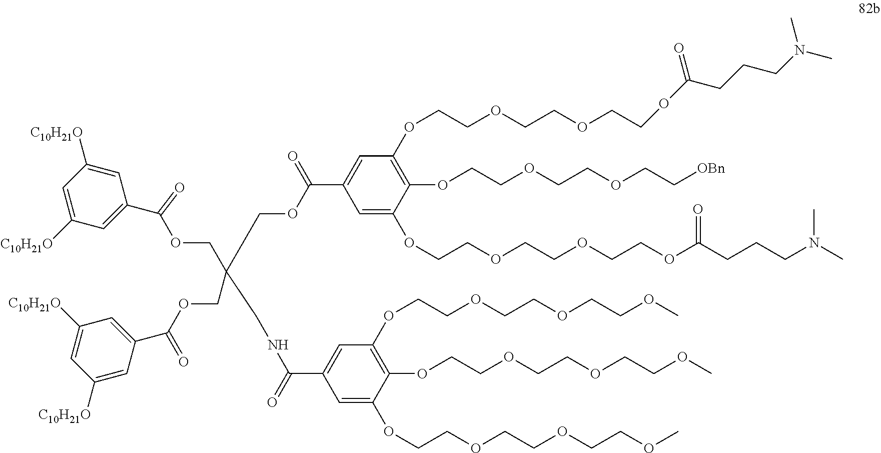

Compound 82b (6/2_{DMBA}^{1,3}_{Bn}²-C10). Compound 82b was synthesized from compound 81b (270 mg, 0.124 mmol), 4-(dimethylamino)butyric acid hydrochloride (52 mg, 0.310 mmol) and DCC (128 mg, 0.620 mmol) following a procedure similar to that used for the synthesis of compound 82a. The title compound was obtained as a light-yellow oil (280 mg, 94%).

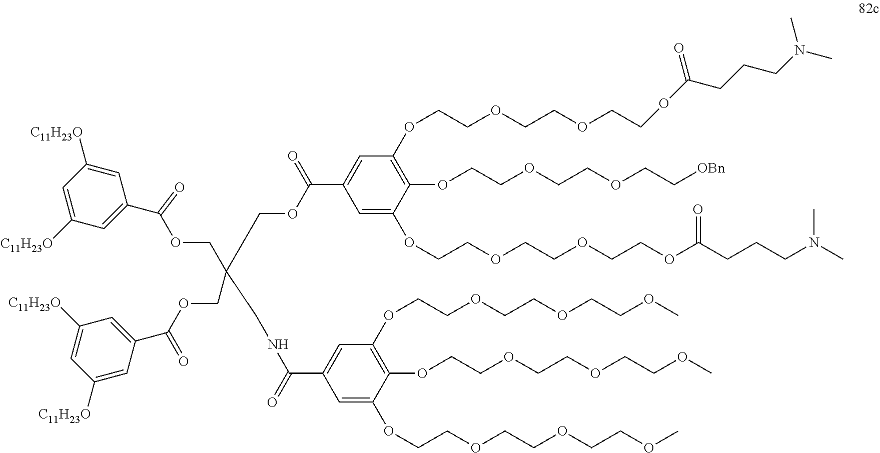

Compound 82c (6/2_{DMBA}^{1,3}_{Bn}²-C11). Compound 82c was synthesized from compound 81c (229 mg, 0.102 mmol), 4-(dimethylamino)butyric acid hydrochloride (42 mg, 0.256 mmol) and DCC (105 mg, 0.510 mmol) following a procedure similar to that used for the synthesis of compound 82a. The title compound was obtained as a light-yellow oil (227 mg, 90%).

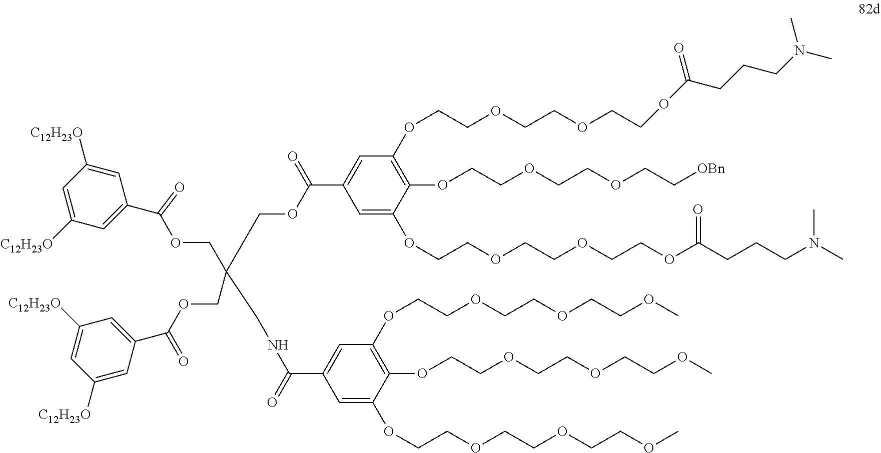

Compound 82d (6/2_{DMBA}^{1,3}_{Bn}²). Compound 82d was synthesized from compound 81d (249 mg, 0.108 mmol), 4-(dimethylamino)butyric acid hydrochloride (46 mg, 0.271 mmol) and DCC (89 mg, 0.432 mmol) following a procedure similar to that used for the synthesis of compound 82a. The title compound was obtained as a light-yellow oil (245 mg, 90%).

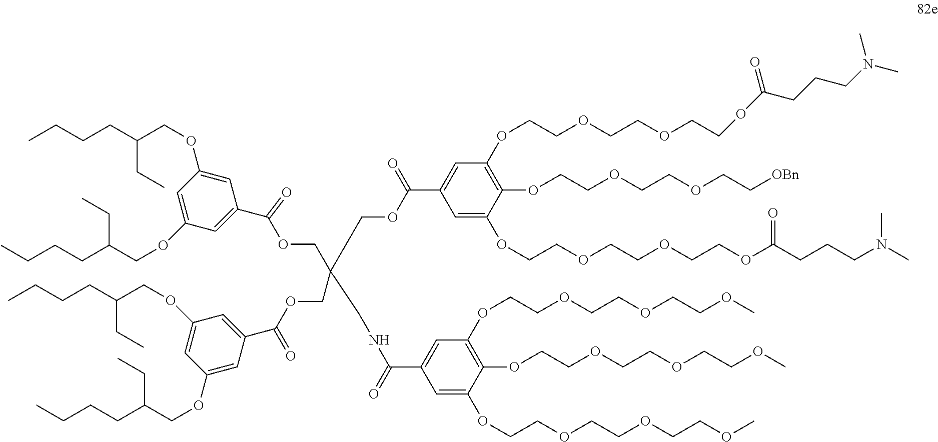 [SMILES: CCCCC(CC)COc1cc(OCC(CC)CCCC)cc(C(=O)OCC(CCOC(=O)c2cc(OCCOCCOCCOC(=O)CCCN(C)C)c(OCCOCCOCCOCc3ccccc3)c(OCCOCCOCCOC(=O)CCCN(C)C)c2)(CNC(=O)c2cc(OCCOCCOCCOC)c(OCCOCCOCCOC)c(OCCOCCOCCOC)c2)COC(=O)c2cc(OCC(CC)CCCC)cc(OCC(CC)CCCC)c2)c1]

Compound 82e (6/2_{DMBA}^{1,3}_{Bn}²-EH). Compound 82e was synthesized from compound 81e (100 mg, 0.048 mmol), 4-(dimethylamino)butyric acid hydrochloride (18 mg, 0.105 mmol) and DCC (30 mg, 0.143 mmol) following a procedure similar to that used for the synthesis of compound 82a. The title compound was obtained as a light-yellow oil (100 mg, 91%).

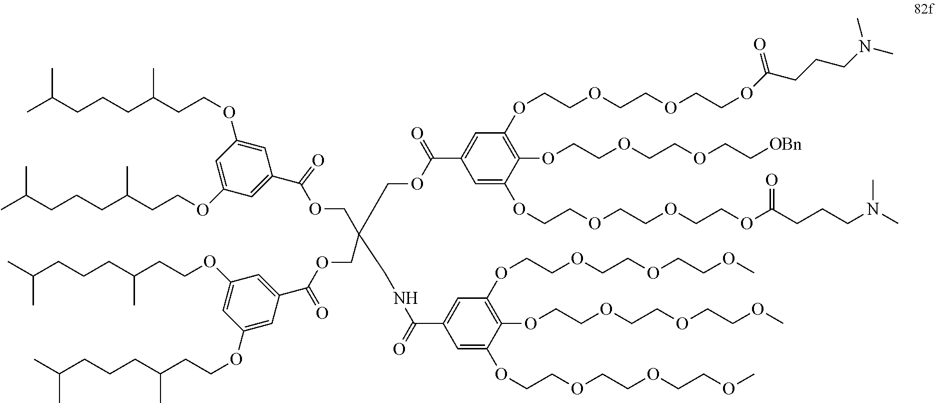 [SMILES: COCCOCCOCCOc1cc(C(=O)NCC(CCOC(=O)c2cc(OCCOCCOCCOC(=O)CCCN(C)C)c(OCCOCCOCCOCc3ccccc3)c(OCCOCCOCCOC(=O)CCCN(C)C)c2)(COC(=O)c2cc(OCCC(C)CCCC(C)C)cc(OCCC(C)CCCC(C)C)c2)COC(=O)c2cc(OCCC(C)CCCC(C)C)cc(OCCC(C)CCCC(C)C)c2)cc(OCCOCCOCCOC)c1OCCOCCOCCOC]

Compound 82f (6/2_{DMBA}^{1,3}_{Bn}²-dm8). Compound 82f was synthesized from compound 81f (215 mg, 0.098 mmol), 4-(dimethylamino)butyric acid hydrochloride (41 mg, 0.246 mmol) and DCC (88 mg, 0.392 mmol) following a procedure similar to that used for the synthesis of compound 82a. The title compound was obtained as a light-yellow oil (203 mg, 86%).

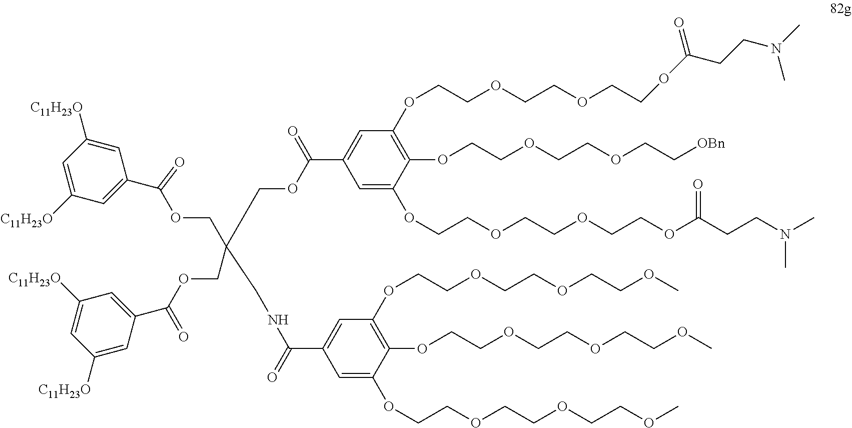

Compound 82g (6/2_{DMPA}^{1,3}_{Bn}²-C11). Compound 82g was synthesized from compound 81c (110 mg, 0.049 mmol), 3-(dimethylamino)propionic acid hydrochloride (17 mg, 0.108 mmol) and DCC (30 mg, 0.147 mmol) following a procedure similar to that used for the synthesis of compound 82a. The title compound was obtained as a light-yellow oil (72 mg, 61%).

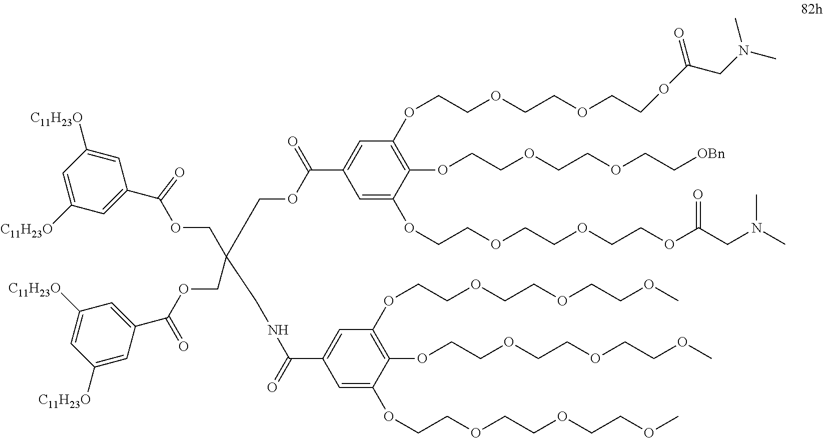

Compound 82h (6/2_{DMA}^{1,3}_{Bn}²-C11). Compound 81c (110 mg, 0.049 mmol) and N,N-dimethylglycine hydrochloride (15 mg, 0.108 mmol) were dissolved in 5 mL dry DCM. DCC (30 mg, 0.147 mmol) was added in one portion into the above mixture. Then 3 drops of TEA were added to the mixture. The reaction mixture was stirred at 23° C. for 12 h and more N,N-dimethylglycine hydrochloride (7.5 mg, 0.054 mmol) was added. The reaction mixture was stirred at 23° C. for another 12 h. The reaction mixture was filtered to remove the urea, which was washed with DCM carefully. The filtrate was concentrated and purified by column chromatography (SiO₂) with DCM/MeOH=30/1, 20/1 and 10/1 as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (58 mg, 49%).

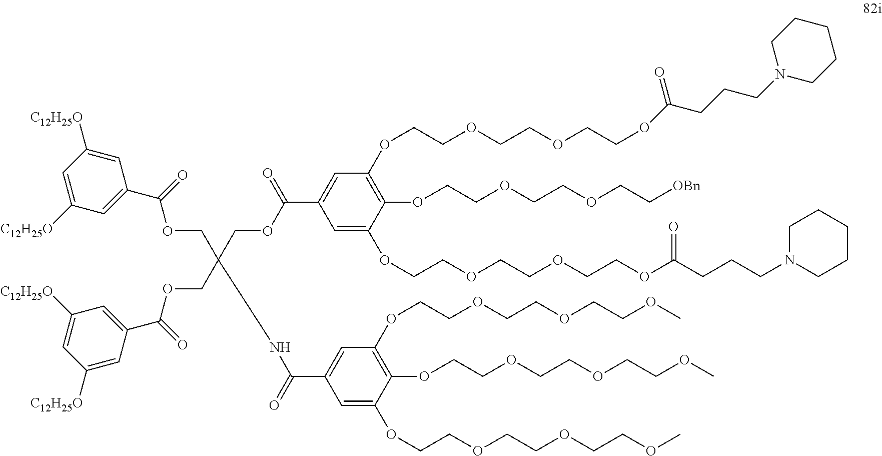

Compound 82i (6/2_{PIP}¹³_{Bn}²). Compound 82i was synthesized from compound 81d (195 mg, 0.085 mmol), compound 56a (32 mg, 0.187 mmol) and DCC (88 mg, 0.424 mmol) following a procedure similar to that used for the synthesis of compound 82a. The title compound was obtained as a light-yellow oil (160 mg, 72%).

### Synthesis of Hybrid Twin-Mix IAJDs 32, 43, 48 of Library 6

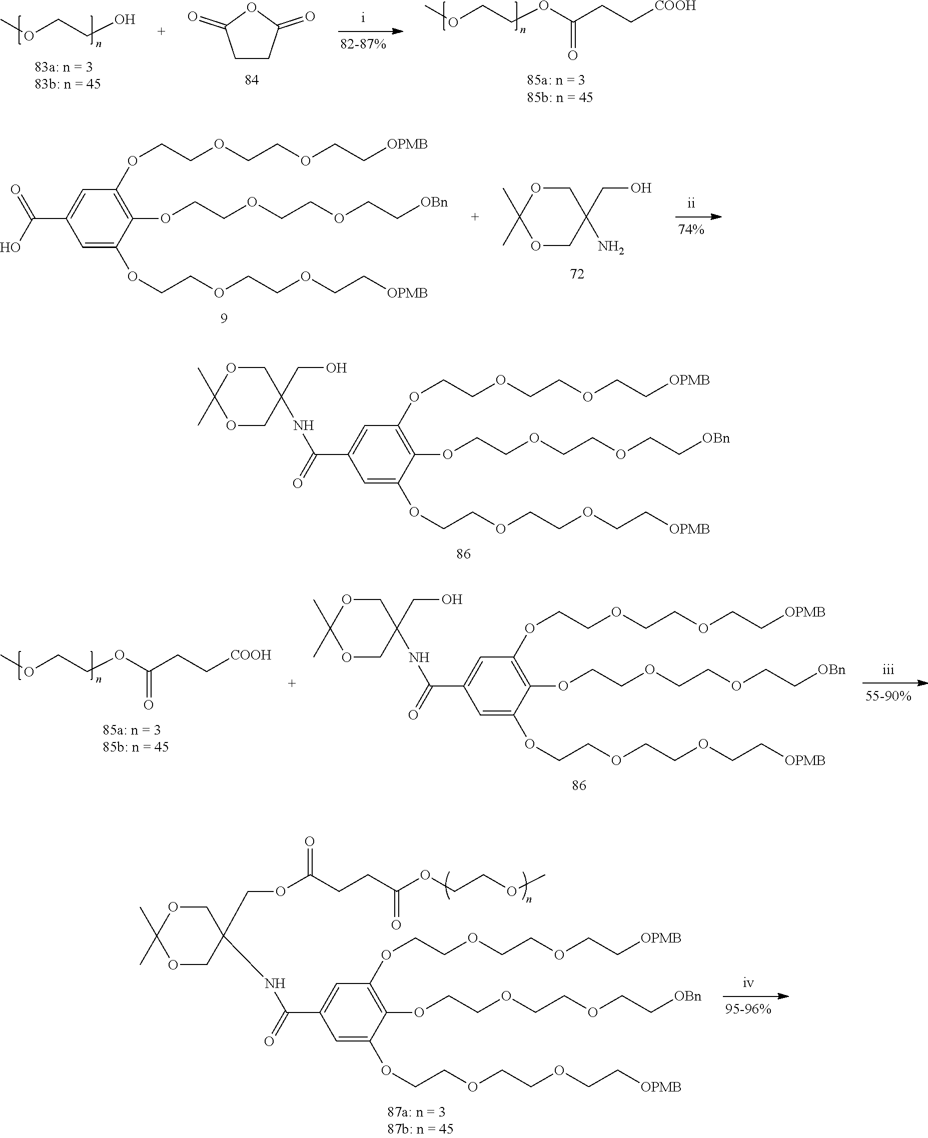

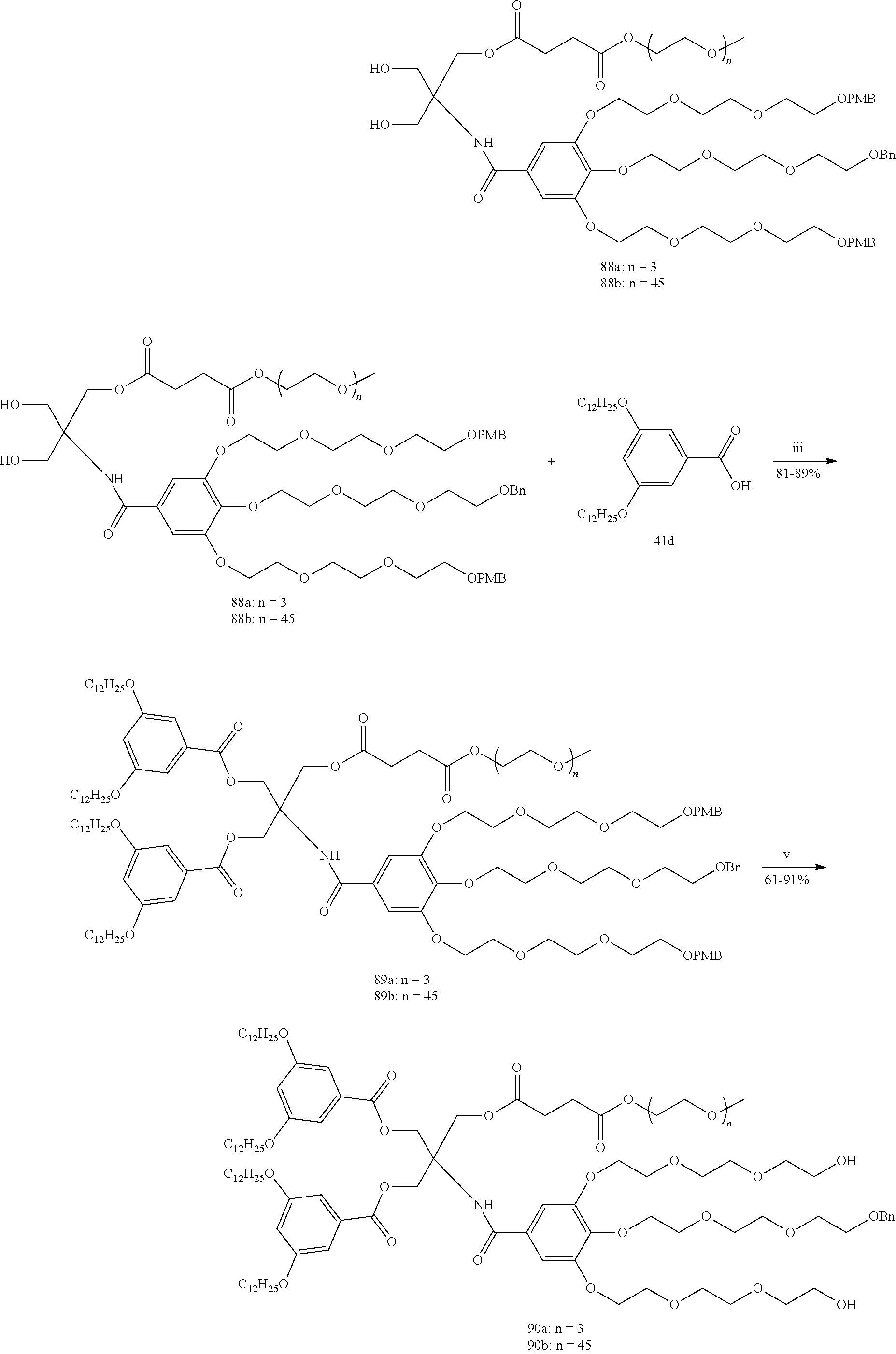

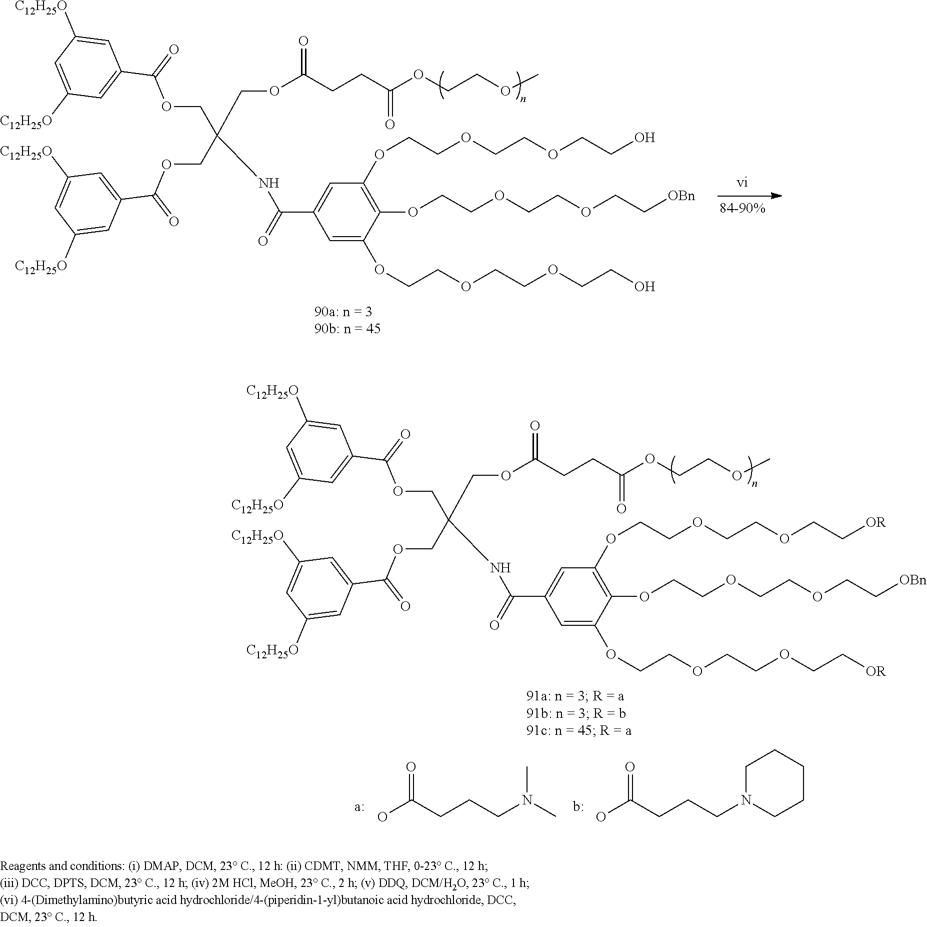

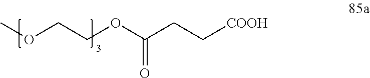

Compound 85a. To a 250 mL flask, DMAP (1.50 g, 12.3 mmol), succinic anhydride (7.32 g, 73.1 mmol), and triethylene glycol monomethyl ether (10.0 g, 60.9 mmol) were dissolved in 50 mL DCM and the reaction mixture was left to react for 12 h at 23° C. The reaction mixture was diluted with 50 mL of DCM, then 25 mL of water was then added, followed by washing 3 times with aqueous 10% NaHSO₄ solution (10 mL). The organic phase was dried over MgSO₄, filtered and concentrated to obtain a colorless oil without further purification (14.08 g, 87%).

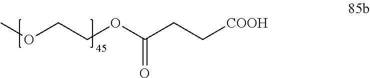

Compound 85b. To a 250 mL flask, DMAP (1.22 g, 10 mmol) and succinic anhydride (1.00 g, 10 mmol) were dissolved in 50 mL DCM. Polyethylene glycol monomethyl ether 2000 (Me-PEG-2000-OH, 2.00 g, 1.0 mmol) was slowly added to the suspension and the reaction mixture was left to react for 12 h at 23° C. The reaction mixture was diluted with 50 mL of DCM, then 100 mL of water was then added, followed by washing 3 times with aqueous 10% NaHSO₄ solution (20 mL). The organic phase was dried over MgSO₄, filtered and concentrated to obtain a white solid without further purification (1.72 g, 82%).

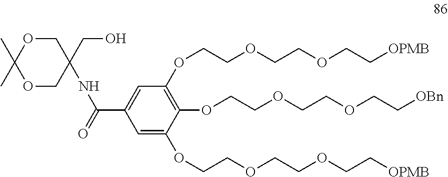

Compound 86. To a THE (10 mL) solution of compound 9 (675 mg, 0.75 mmol) and compound 72 (136 mg, 0.84 mmol) was added CDMT (148 mg, 0.84 mmol). The mixture was cooled to 0° C. and N-methylmorpholine (NMM, 195 mg, 1.92 mmol) was added under N₂. The reaction mixture was allowed to stir at 23° C. for 12 h. The precipitate was then filtered, and the crude product was further purified by column chromatography on silica gel with a mobile phase of DCM/MeOH=20/1 to yield the title compound as a colorless oil (578 mg, 74%).

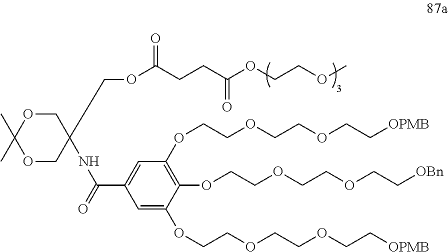

Compound 87a. To a DCM (10 mL) solution of compound 85a (179 mg, 0.677 mmol), compound 86 (640 mg, 0.615 mmol) and DPTS (271 mg, 0.922 mmol), was added DCC (380 mg, 1.85 mmol). The mixture was allowed to stir at 23° C. for 12 h. The precipitate was filtered, and the filtrate was concentrated to dryness. The crude product was further purified by column 20 chromatography with a mobile phase of DCM/MeOH=20/1 to yield the title compound as a colorless viscous liquid (710 mg, 90%).

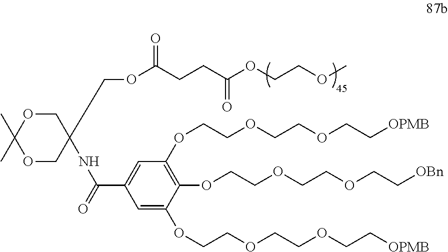

Compound 87b. To a DCM (10 mL) solution of compound 85b (1.17 g, 0.557 mmol), compound 86 (578 mg, 0.557 mmol) and DPTS (180 mg, 0.613 mmol), was added DCC (287 mg, 1.40 mmol). The mixture was allowed to stir at 23° C. for 12 h. The precipitate was filtered, and the filtrate was concentrated to dryness. The crude product was further purified by column chromatography with a mobile phase of DCM/MeOH=20/1 to yield the title compound as a colorless viscous liquid (960 mg, 55%).

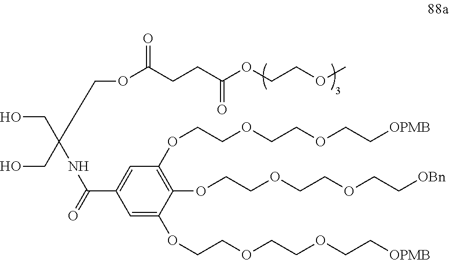

Compound 88a. To a MeOH solution (10 mL) of compound 87a (710 mg, 0.552 mmol) was added 3 mL of HCl aqueous solution (2 M). The mixture was allowed to stir at 23° C. for 2 h and saturated NaHCO₃solution (20 mL) was added. The mixture was extracted with DCM (50 mL) for 3 times. An organic extract was dried over anhydrous MgSO₄ and evaporated to dryness under reduced pressure to yield the title compound as a colorless viscous liquid without further purification (653 mg, 95%).

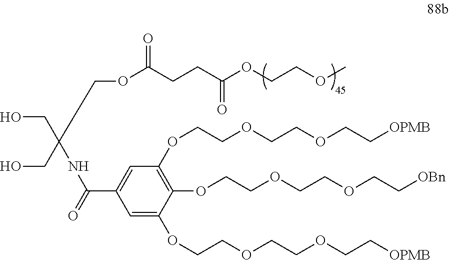

Compound 88b. Compound 88b was synthesized from compound 87b (960 mg, 0.307 mmol) following a procedure similar to that used for the synthesis of compound 88a. The title compound was obtained as a colorless viscous liquid without further purification (930 mg, 96%).

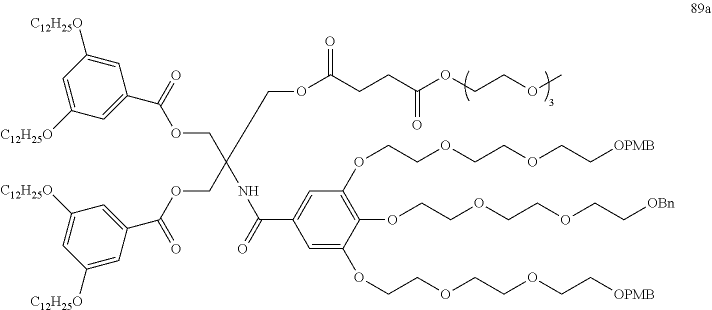

Compound 89a. To a DCM (10 mL) solution of compound 41d (539 mg, 1.10 mmol), compound 88a (653 mg, 0.524 mmol) and DPTS (384 mg, 1.31 mmol), was added DCC (541 mg, 2.62 mmol). The mixture was allowed to stir at 23° C. for 12 h. The precipitate was filtered, and the filtrate was concentrated to dryness. The crude product was further purified by column chromatography with a mobile phase of DCM/MeOH=20/1 to yield the title compound as a colorless viscous liquid (933 mg, 81%).

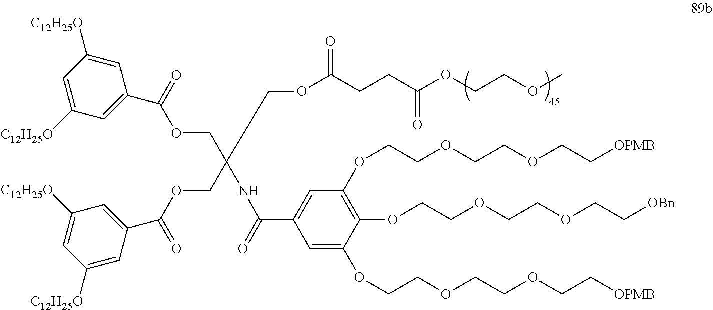

Compound 89b. To a DCM (10 mL) solution of compound 41d (106 mg, 0.220 mmol), compound 88b (302 mg, 0.098 mmol) and DPTS (74 mg, 0.250 mmol), was added DCC (103 mg, 0.500 mmol). The mixture was allowed to stir at 23° C. for 12 h. The precipitate was filtered, and the filtrate was concentrated to dryness. The crude product was further purified by column chromatography with a mobile phase of DCM/MeOH=20/1 to yield the title compound as a colorless viscous liquid (351 mg, 89%).

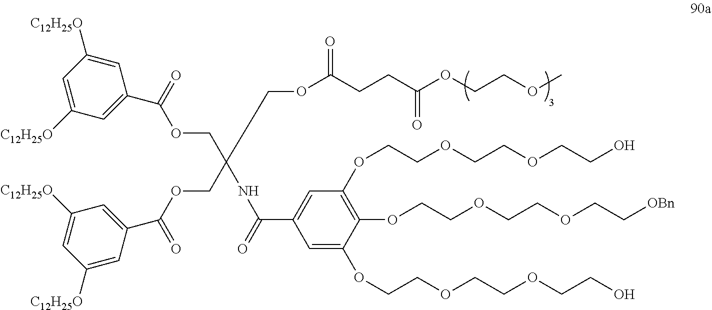

Compound 90a. To a mixed solution of DCM (15 mL) and water (0.75 mL) of compound 89a (933 mg, 0.426 mmol), was added DDQ (241 mg, 1.06 mmol). The mixture was allowed to stir at 23° C. for 3 h. The precipitates were filtered out and DCM (20 mL) was added. The mixture was washed by NaHCO₃aqueous solution (saturated), NaHSO₃ aqueous solution (2%) and NaHCO₃aqueous solution (saturated) successively. The organic phase was dried over anhydrous MgSO₄ and filtered. The filtrate was concentrated and purified by column chromatography with a mobile phase of DCM/MeOH=20/1 to yield the title compound as a colorless viscous liquid (504 mg, 61%).

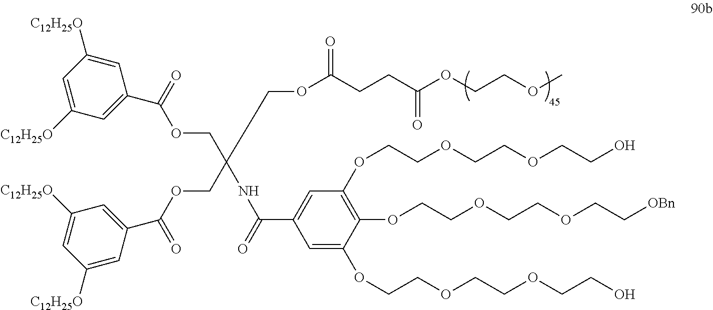

Compound 90b. To a mixed solution of DCM (5 mL) and water (0.25 mL) of compound 90a (270 mg, 0.067 mmol), was added DDQ (51 mg, 0.220 mmol). The mixture was allowed to stir at 23° C. for 2 h. The completion of the reaction was monitored by ¹H NMR when the signal at 6=4.46 from the p-methoxy benzyl groups was completely disappeared. The precipitates were filtered out and DCM (20 mL) was added. The mixture was washed by NaHCO₃aqueous solution (saturated), NaHSO₃ aqueous solution (2%) and NaHCO₃aqueous solution (saturated) successively. The organic phase was dried over anhydrous MgSO₄ and filtered. The filtrate was concentrated and purified by column chromatography with a mobile phase of EtOAc/MeOH=8/1 to yield the title compound as a colorless viscous liquid (236 mg, 91%).

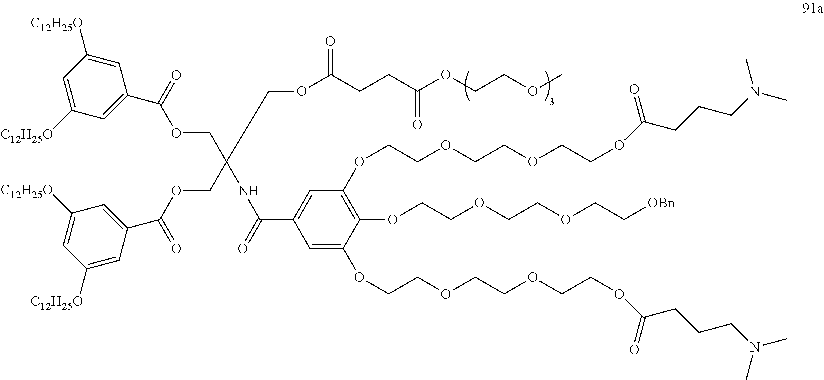

Compound 91a (4/2_{DMBA}^{1,3}_{Bn}²). Compound 90a (205 mg, 0.105 mmol) and 4-(dimethylamino)butyric acid hydrochloride (44 mg, 0.263 mmol) were dissolved in 5 mL dry DCM. DCC (108 mg, 0.525 mmol) was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with DCM/MeOH=10/1, 6/1 with 0.1% TEA as the eluent. Then the crude product was dissolved in NaHCO₃solution (2%, 30 mL) and the mixture was extracted by DCM (30 mL×3). The organic phase was dried over anhydrous MgSO₄. Filtration and evaporation of the solvent yielded the title compound as a light-yellow oil (205 mg, 90%).

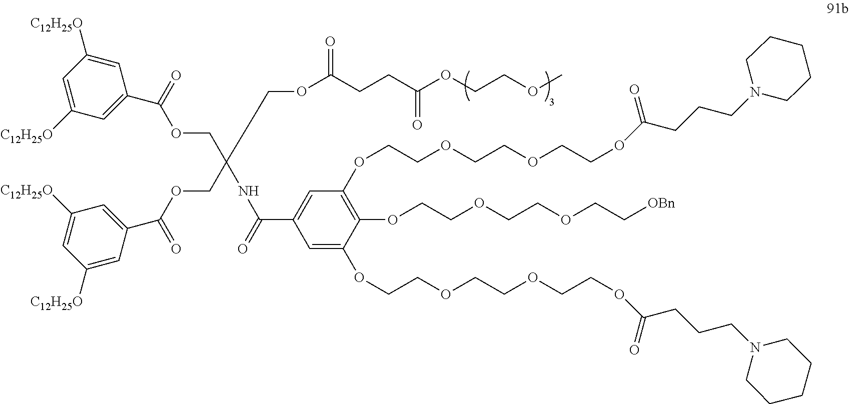

Compound 91b (4/2_{PIP}^{1,3}_{Bn}²). Compound 91b was synthesized from compound 90a (154 mg, 0.079 mmol), compound 56a (44 mg, 0.263 mmol) and DCC (33 mg, 0.193 mmol) following a procedure similar to that used for the synthesis of compound 91a. The title compound was obtained as a light-yellow oil (160 mg, 90%).

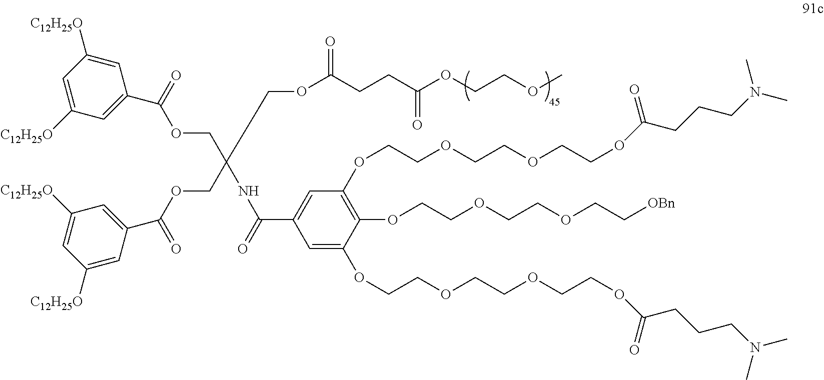

Compound 91c (4/2_{DMBA}^{1,3}_{Bn}²_{PEG}⁴). Compound 91c was synthesized from compound 90b (230 mg, 0.061 mmol), 4-(dimethylamino)butyric acid hydrochloride (26 mg, 0.150 mmol) and DCC (62 mg, 0.300 mmol) following a procedure similar to that used for the synthesis of compound 91a. The title compound was obtained as a light-yellow solid (209 mg, 84%). Dimensions of DNPs Containing Luc-mRNA Obtained by Co-assembly of IAJDs from Library 1 with Luc-mRNA by Injection in Citrate Buffer (10 mM, pH 3.0) or Acetate Buffer (10 mM, pH 4.0/5.2) and Luminescence Expression in HEK293T Cells (125 ng/well) are presented in Table 1.

TABLE 1

| | | Buffer | C_{IAJD} | C_{Luc-mRNA} | Size | | | Standard |
|---|---|---|---|---|---|---|---|---|
| IAJD | | (10 mM) | (mg/mL) | (mg/mL) | (d, nm) | PDI | Luminescence | deviation |
| Library 1 | 1 | Citrate | 4 | 0.05 | 73 | 0.406 | 552,723 | 83,190 |
| Single- | | (pH 3.0) | | | | | | |
| Single | 1 | Acetate | 4 | 0.05 | 85 | 0.282 | 1,009,268 | 180,480 |
| | | (pH 4.0) | | | | | | |
| | 1 | Acetate | 4 | 0.10 | 295 | 0.501 | 183,282 | 55,508 |
| | | (pH 5.2) | | | | | | |
| | 2 | Citrate | 4 | 0.05 | 94 | 0.530 | 165,500 | 15,360 |
| | | (pH 3.0) | | | | | | |
| | 2 | Acetate | 4 | 0.05 | 83 | 0.254 | 122,687 | 38,579 |
| | | (pH 4.0) | | | | | | |
| | 2 | Acetate | 4 | 0.10 | 289 | 0.521 | 71,265 | 43,096 |
| | | (pH 5.2) | | | | | | |
| | 3 | Citrate | 4 | 0.05 | 70 | 0.311 | 7,093 | 1,946 |
| | | (pH 3.0) | | | | | | |
| | 3 | Acetate | 4 | 0.05 | 65 | 0.272 | 4,412 | 199 |
| | | (pH 4.0) | | | | | | |
| | 3 | Acetate | 4 | 0.10 | 95 | 0.244 | 3,581 | 556 |
| | | (pH 5.2) | | | | | | |
| | 4 | Citrate | 4 | 0.05 | 61 | 0.460 | 2,063 | 82 |
| | | (pH 3.0) | | | | | | |
| | 4 | Acetate | 4 | 0.05 | 58 | 0.330 | 3,283 | 1,981 |
| | | (pH 4.0) | | | | | | |
| | 4 | Acetate | 4 | 0.10 | 178 | 0.284 | 1,613 | 156 |
| | | (pH 5.2) | | | | | | |
| | 5 | Citrate | 4 | 0.05 | 77 | 0.320 | 18,939 | 4,425 |
| | | (pH 3.0) | | | | | | |
| | 5 | Acetate | 4 | 0.05 | 60 | 0.291 | 17,156 | 12,967 |
| | | (pH 4.0) | | | | | | |
| | 5 | Acetate | 4 | 0.10 | 156 | 0.281 | 1,226 | 238 |
| | | (pH 5.2) | | | | | | |
| | 6 | Citrate | 4 | 0.05 | 91 | 0.353 | 15,207 | 1,536 |
| | | (pH 3.0) | | | | | | |
| | 6 | Acetate | 4 | 0.05 | 59 | 0.291 | 6,845 | 1,073 |
| | | (pH 4.0) | | | | | | |
| | 6 | Acetate | 4 | 0.10 | 92 | 0.246 | 33,562 | 4,359 |
| | | (pH 5.2) | | | | | | |
| | 7 | Citrate | 4 | 0.05 | 100 | 0.406 | 10,119 | 1,374 |
| | | (pH 3.0) | | | | | | |
| | 7 | Acetate | 4 | 0.05 | 86 | 0.329 | 1,810 | 43 |
| | | (pH 4.0) | | | | | | |
| | 7 | Acetate | 4 | 0.10 | 132 | 0.338 | 9,162 | 801 |
| | | (pH 5.2) | | | | | | |
| | 8 | Citrate | 4 | 0.05 | 73 | 0.442 | 1,291,219 | 204,977 |
| | | (pH 3.0) | | | | | | |
| | 8 | Acetate | 4 | 0.05 | 70 | 0.295 | 1,975,892 | 540,459 |
| | | (pH 4.0) | | | | | | |
| | 8 | Acetate | 4 | 0.10 | 142 | 0.269 | 1,473,247 | 158,493 |
| | | (pH 5.2) | | | | | | |
| | 9 | Citrate | 4 | 0.05 | 87 | 0.271 | 4,236,960 | 428,817 |
| | | (pH 3.0) | | | | | | |
| | 9 | Acetate | 4 | 0.05 | 55 | 0.306 | 4,474,957 | 215,800 |
| | | (pH 4.0) | | | | | | |
| | 9 | Acetate | 4 | 0.10 | 98 | 0.224 | 4,390,218 | 197,649 |
| | | (pH 5.2) | | | | | | |
| Negative | | | | | | | 81 | 5 |
| MC3 | | | | | | | 1,320,533 | 105,967 |
| TransIT | | | | | | | 3,642,765 | 484,480 |

Dimensions of DNPs Containing Luc-mRNA Obtained by Co-assembly of IAJDs from Library 2-4 with Luc-mRNA by Injection in Citrate Buffer (10 mM, pH 3.0) or Acetate Buffer (10 mM, pH 4.0/5.2) and Luminescence Expression in HEK293T Cells (125 ng/well) are presented in Table 2.

TABLE 2

| | | Buffer | C_{IAJD} | C_{Luc-mRNA} | Size | | | Standard |
|---|---|---|---|---|---|---|---|---|
| IAJD | | (10 mM) | (mg/mL) | (mg/mL) | (d, nm) | PDI | Luminescence | deviation |
| Library 2 | 19 | Citrate | 4 | 0.05 | 77 | 0.271 | 39 | 10 |
| Single- | | (pH 3.0) | | | | | | |
| Single | 19 | Acetate | 4 | 0.05 | 65 | 0.274 | 39 | 7 |
| | | (pH 4.0) | | | | | | |
| | 19 | Acetate | 4 | 0.10 | 110 | 0.167 | 33 | 7 |
| | | (pH 5.2) | | | | | | |
| | 20 | Citrate | 4 | 0.05 | 72 | 0.293 | 249 | 89 |
| | | (pH 3.0) | | | | | | |
| | 20 | Acetate | 4 | 0.05 | 62 | 0.386 | 28 | 3 |
| | | (pH 4.0) | | | | | | |
| | 20 | Acetate | 4 | 0.10 | 120 | 0.203 | 47,808 | 8,089 |
| | | (pH 5.2) | | | | | | |
| | 21 | Citrate | 4 | 0.05 | 65 | 0.414 | 927,496 | 97,565 |
| | | (pH 3.0) | | | | | | |
| | 21 | Acetate | 4 | 0.05 | 68 | 0.301 | 1,098,558 | 149,158 |
| | | (pH 4.0) | | | | | | |
| | 21 | Acetate | 4 | 0.10 | 102 | 0.200 | 2,901,220 | 237,377 |
| | | (pH 5.2) | | | | | | |
| | 22 | Citrate | 4 | 0.05 | 69 | 0.296 | 4,740,529 | 464,414 |
| | | (pH 3.0) | | | | | | |
| | 22 | Acetate | 4 | 0.05 | 66 | 0.280 | 4,369,979 | 1,133,074 |
| | | (pH 4.0) | | | | | | |
| | 22 | Acetate | 4 | 0.10 | 114 | 0.232 | 4,664,358 | 733,410 |
| | | (pH 5.2) | | | | | | |
| | 23 | Citrate | 4 | 0.05 | 86 | 0.213 | 159 | 10 |
| | | (pH 3.0) | | | | | | |
| | 23 | Acetate | 4 | 0.05 | 68 | 0.328 | 38 | 8 |
| | | (pH 4.0) | | | | | | |
| | 23 | Acetate | 4 | 0.10 | 108 | 0.230 | 44 | 13 |
| | | (pH 5.2) | | | | | | |
| | 24 | Citrate | 4 | 0.05 | 64 | 0.421 | 5,578 | 3,523 |
| | | (pH 3.0) | | | | | | |
| | 24 | Acetate | 4 | 0.05 | 50 | 0.311 | 27,766 | 9,240 |
| | | (pH 4.0) | | | | | | |
| | 24 | Acetate | 4 | 0.10 | 108 | 0.216 | 339,002 | 27,855 |
| | | (pH 5.2) | | | | | | |
| | 25 | Citrate | 4 | 0.05 | 117 | 0.538 | 116,292 | 8,791 |
| | | (pH 3.0) | | | | | | |
| | 25 | Acetate | 4 | 0.05 | 64 | 0.425 | 5,658 | 1,859 |
| | | (pH 4.0) | | | | | | |
| | 25 | Acetate | 4 | 0.10 | 147 | 0.202 | 57,464 | 7,513 |
| | | (pH 5.2) | | | | | | |
| Library 3 | 44 | Citrate | 4 | 0.05 | 85 | 0.310 | 66,956 | 3,183 |
| Single- | | (pH 3.0) | | | | | | |
| Single | 44 | Acetate | 4 | 0.05 | 98 | 0.279 | 84,746 | 5,790 |
| | | (pH 4.0) | | | | | | |
| | 44 | Acetate | 4 | 0.10 | 171 | 0.252 | 99,996 | 103,05 |
| | | (pH 5.2) | | | | | | |
| | 33 | Citrate | 4 | 0.05 | 74 | 0.278 | 36,871 | 18,033 |
| | | (pH 3.0) | | | | | | |
| | 33 | Acetate | 4 | 0.05 | 72 | 0.260 | 32,404 | 24,064 |
| | | (pH 4.0) | | | | | | |
| | 33 | Acetate | 4 | 0.10 | 93 | 0.216 | 100,259 | 11,162 |
| | | (pH 4.0) | | | | | | |
| | 34 | Citrate | 4 | 0.05 | 83 | 0.264 | 37,381 | 4,560 |
| | | (pH 3.0) | | | | | | |
| | 34 | Acetate | 4 | 0.05 | 56 | 0.255 | 52,554 | 36,103 |
| | | (pH 4.0) | | | | | | |
| | 34 | Acetate | 4 | 0.10 | 83 | 0.245 | 118,838 | 35,286 |
| | | (pH 4.0) | | | | | | |
| | 45 | Citrate | 4 | 0.05 | 64 | 0.431 | 22 | 12 |
| | | (pH 3.0) | | | | | | |
| | 45 | Acetate | 4 | 0.05 | 102 | 0.267 | 32 | 5 |
| | | (pH 4.0) | | | | | | |
| | 45 | Acetate | 4 | 0.10 | 512 | 0.536 | 15 | 6 |
| | | (pH 5.2) | | | | | | |
| | 35 | Citrate | 4 | 0.05 | 69 | 0.277 | 58 | 11 |
| | | (pH 3.0) | | | | | | |
| | 35 | Acetate | 4 | 0.05 | 53 | 0.260 | 76 | 13 |
| | | (pH 4.0 | | | | | | |
| | 35 | Acetate | 4 | 0.10 | 70 | 0.245 | 78 | 41 |
| | | (pH 4.0) | | | | | | |
| | 36 | Citrate | 4 | 0.05 | 58 | 0.269 | 42 | 14 |
| | | (pH 3.0) | | | | | | |
| | 36 | Acetate | 4 | 0.05 | 53 | 0.265 | 46 | 19 |
| | | (pH 4.0) | | | | | | |
| | 36 | Acetate | 4 | 0.10 | 71 | 0.224 | 83 | 22 |
| | | (pH 5.2) | | | | | | |
| Library 4 | 30 | Citrate | 4 | 0.05 | 234 | 0.202 | 662 | 142 |
| Single- | | (pH 3.0) | | | | | | |
| Single | 30 | Acetate | 4 | 0.05 | 73 | 0.321 | 5,421 | 1,812 |
| | | (pH 4.0) | | | | | | |
| | 30 | Acetate | 4 | 0.10 | 165 | 0.377 | 674 | 52 |
| | | (pH 5.2) | | | | | | |
| | 31 | Citrate | 4 | 0.05 | 166 | 0.881 | 16,166 | 4,370 |
| | | (pH 3.0) | | | | | | |
| | 31 | Acetate | 4 | 0.05 | 76 | 0.489 | 41,889 | 5,247 |
| | | (pH 4.0) | | | | | | |
| | 31 | Acetate | 4 | 0.10 | 210 | 0.261 | 28,524 | 3,995 |
| | | (pH 5.2) | | | | | | |
| | 37 | Citrate | 4 | 0.05 | 118 | 0.526 | 3,800 | 265 |
| | | (pH 3.0) | | | | | | |
| | 37 | Acetate | 4 | 0.05 | 79 | 0.329 | 30,785 | 4,803 |
| | | (pH 4.0) | | | | | | |
| | 37 | Acetate | 4 | 0.10 | 131 | 0.267 | 2,606 | 489 |
| | | (pH 4.0) | | | | | | |

Dimensions of DNPs Containing Luc-mRNA Obtained by Co-assembly of IAJDs from Library 5 with Luc-mRNA by Injection in Citrate Buffer (10 mM, pH 3.0) or Acetate Buffer (10 mM, pH 4.0/5.2) and Luminescence Expression in HEK293T Cells (125 ng/well) are presented in Table 3.

TABLE 3

| | | Buffer | C_{IAJD} | C_{Luc-mRNA} | Size | | | Standard |
|---|---|---|---|---|---|---|---|---|
| IAJD | | (10 mM) | (mg/mL) | (mg/mL) | (d, nm) | PDI | Luminescence | deviation |
| Library 5 | 10 | Citrate | 4 | 0.05 | 110 | 0.467 | 52,530 | 6,718 |
| Twin-Twin | | (pH 3.0) | | | | | | |
| | 10 | Acetate | 4 | 0.05 | 92 | 0.242 | 64,605 | 33,615 |
| | | (pH 4.0) | | | | | | |
| | 10 | Acetate | 4 | 0.10 | 347 | 0.449 | 131,357 | 22,629 |
| | | (pH 5.2) | | | | | | |
| | 11 | Citrate | 4 | 0.05 | 108 | 0.464 | 26,378 | 5,030 |
| | | (pH 3.0) | | | | | | |
| | 11 | Acetate | 4 | 0.05 | 90 | 0.272 | 26,843 | 8,193 |
| | | (pH 4.0) | | | | | | |
| | 11 | Acetate | 4 | 0.10 | 369 | 0.477 | 24,728 | 10,049 |
| | | (pH 5.2) | | | | | | |
| | 12 | Citrate | 4 | 0.05 | 66 | 0.473 | 87 | 33 |
| | | (pH 3.0) | | | | | | |
| | 12 | Acetate | 4 | 0.05 | 64 | 0.326 | 71 | 11 |
| | | (pH 4.0) | | | | | | |
| | 12 | Acetate | 4 | 0.10 | 171 | 0.467 | 331 | 82 |
| | | (pH 5.2) | | | | | | |
| | 13 | Citrate | 4 | 0.05 | 66 | 0.331 | 83 | 44 |
| | | (pH 3.0) | | | | | | |
| | 13 | Acetate | 4 | 0.05 | 63 | 0.319 | 49 | 8 |
| | | (pH 4.0) | | | | | | |
| | 13 | Acetate | 4 | 0.10 | 163 | 0.299 | 251 | 92 |
| | | (pH 5.2) | | | | | | |
| | 14 | Citrate | 4 | 0.05 | 75 | 0.474 | 187 | 121 |
| | | (pH 3.0) | | | | | | |
| | 14 | Acetate | 4 | 0.05 | 64 | 0.456 | 77 | 41 |
| | | (pH 4.0) | | | | | | |
| | 14 | Acetate | 4 | 0.10 | 98 | 0.206 | 38 | 10 |
| | | (pH 5.2) | | | | | | |
| | 15 | Citrate | 4 | 0.05 | 76 | 0.431 | 18,158 | 10,971 |
| | | (pH 3.0) | | | | | | |
| | 15 | Acetate | 4 | 0.05 | 84 | 0.265 | 14,233 | 1,705 |
| | | (pH 4.0) | | | | | | |
| | 15 | Acetate | 4 | 0.10 | 103 | 0.262 | 46,433 | 11,414 |
| | | (pH 5.2) | | | | | | |
| | 16 | Citrate | 4 | 0.05 | 77 | 0.429 | 12,299 | 2,475 |
| | | (pH 3.0) | | | | | | |
| | 16 | Acetate | 4 | 0.05 | 56 | 0.270 | 7,789 | 2,103 |
| | | (pH 4.0) | | | | | | |
| | 16 | Acetate | 4 | 0.10 | 108 | 0.183 | 27,883 | 7,388 |
| | | (pH 5.2) | | | | | | |
| | 17 | Citrate | 4 | 0.05 | 82 | 0.398 | 32,239 | 11,612 |
| | | (pH 3.0) | | | | | | |
| | 17 | Acetate | 4 | 0.05 | 57 | 0.472 | 34,120 | 5,598 |
| | | (pH 4.0) | | | | | | |
| | 17 | Acetate | 4 | 0.10 | 203 | 0.449 | 19,080 | 3,288 |
| | | (pH 5.2) | | | | | | |
| | 18 | Citrate | 4 | 0.05 | 69 | 0.333 | 368,727 | 62,081 |
| | | (pH 3.0) | | | | | | |
| | 18 | Acetate | 4 | 0.05 | 52 | 0.325 | 478,097 | 29,427 |
| | | (pH 4.0) | | | | | | |
| | 18 | Acetate | 4 | 0.10 | 156 | 0.370 | 161,286 | 14,020 |
| | | (pH 5.2) | | | | | | |
| | 46 | Citrate | 4 | 0.05 | 71 | 0.433 | 186,720 | 60,386 |
| | | (pH 3.0) | | | | | | |
| | 46 | Acetate | 4 | 0.05 | 78 | 0.293 | 101,862 | 45,648 |
| | | (pH 4.0) | | | | | | |
| | 46 | Acetate | 4 | 0.10 | 111 | 0.217 | 2,819 | 2,466 |
| | | (pH 4.0) | | | | | | |

Dimensions of DNPs Containing Luc-mRNA Obtained by Co-assembly of IAJDs from Library 6 with Luc-mRNA by Injection in Citrate Buffer (10 mM, pH 3.0) or Acetate Buffer (10 mM, pH 4.0/5.2) and Luminescence Expression in HEK293T Cells (125 ng/well) are presented in Table 4.

TABLE 4

| | | Buffer | C_{IAJD} | C_{Luc-mRNA} | Size | | | Standard |
|---|---|---|---|---|---|---|---|---|
| IAJD | | (10 mM) | (mg/mL) | (mg/mL) | (d, nm) | PDI | Luminescence | deviation |
| Library 6 | 38 | Citrate | 4 | 0.05 | 96 | 0.341 | 32 | 5 |
| Hybrid | | (pH 3.0) | | | | | | |
| Twin-Mix | 38 | Acetate | 4 | 0.05 | 119 | 0.353 | 45 | 17 |
| | | (pH 4.0) | | | | | | |
| | 38 | Acetate | 4 | 0.10 | 418 | 0.364 | 570 | 206 |
| | | (pH 5.2) | | | | | | |
| | 39 | Citrate | 4 | 0.05 | 117 | 0.430 | 59 | 9 |
| | | (pH 3.0) | | | | | | |
| | 39 | Acetate | 4 | 0.05 | 147 | 0.478 | 74 | 13 |
| | | (pH 4.0) | | | | | | |
| | 39 | Acetate | 4 | 0.10 | 242 | 0.392 | 311 | 88 |
| | | (pH 5.2) | | | | | | |
| | 40 | Citrate | 4 | 0.05 | 98 | 0.531 | 9,184 | 1,028 |
| | | (pH 3.0) | | | | | | |
| | 40 | Acetate | 4 | 0.05 | 73 | 0.439 | 12,389 | 1,296 |
| | | (pH 4.0) | | | | | | |
| | 40 | Acetate | 4 | 0.10 | 423 | 0.388 | 11,392 | 1,209 |
| | | (pH 5.2) | | | | | | |
| | 41 | Citrate | 4 | 0.05 | 107 | 0.509 | 10,079 | 1,494 |
| | | (pH 3.0) | | | | | | |
| | 41 | Acetate | 4 | 0.05 | 104 | 0.220 | 38,864 | 6,688 |
| | | (pH 4.0) | | | | | | |
| | 41 | Acetate | 4 | 0.10 | 254 | 0.517 | 45,553 | 4,585 |
| | | (pH 5.2) | | | | | | |
| | 42 | Citrate | 4 | 0.05 | 59 | 0.418 | 637 | 43 |
| | | (pH 3.0) | | | | | | |
| | 42 | Acetate | 4 | 0.05 | 50 | 0.323 | 4,357 | 90 |
| | | (pH 4.0) | | | | | | |
| | 42 | Acetate | 4 | 0.10 | 307 | 0.570 | 5,137 | 2,297 |
| | | (pH 5.2) | | | | | | |
| | 49 | Citrate | 4 | 0.05 | 70 | 0.356 | 7,925 | 2,909 |
| | | (pH 3.0) | | | | | | |
| | 49 | Acetate | 4 | 0.05 | 72 | 0.253 | 7,908 | 1,832 |
| | | (pH 4.0) | | | | | | |
| | 49 | Acetate | 4 | 0.10 | 242 | 0.340 | 4,632 | 1,337 |
| | | (pH 5.2) | | | | | | |
| | 26 | Citrate | 4 | 0.05 | 74 | 0.310 | 330,183 | 5,311 |
| | | (pH 3.0) | | | | | | |
| | 26 | Acetate | 4 | 0.05 | 75 | 0.296 | 598,804 | 98,063 |
| | | (pH 4.0 | | | | | | |
| | 26 | Acetate | 4 | 0.10 | 227 | 0.448 | 184,041 | 28,499 |
| | | (pH 5.2) | | | | | | |
| | 27 | Citrate | 4 | 0.05 | 72 | 0.449 | 246,051 | 23,408 |
| | | (pH 3.0) | | | | | | |
| | 27 | Acetate | 4 | 0.05 | 78 | 0.266 | 330,414 | 133,482 |
| | | (pH 4.0) | | | | | | |
| | 27 | Acetate | 4 | 0.10 | 306 | 0.538 | 81,351 | 11,532 |
| | | (pH 5.2) | | | | | | |
| | 28 | Citrate | 4 | 0.05 | 87 | 0.341 | 87,848 | 14,593 |
| | | (pH 3.0) | | | | | | |
| | 28 | Acetate | 4 | 0.05 | 76 | 0.284 | 114,911 | 15,142 |
| | | (pH 4.0) | | | | | | |
| | 28 | Acetate | 4 | 0.10 | 266 | 0.488 | 66,366 | 6,091 |
| | | (pH 5.2) | | | | | | |
| | 50 | Citrate | 4 | 0.05 | 86 | 0.386 | 6,871 | 2,245 |
| | | (pH 3.0) | | | | | | |
| | 50 | Acetate | 4 | 0.05 | 77 | 0.376 | 10,925 | 3,855 |
| | | (pH 4.0) | | | | | | |
| | 50 | Acetate | 4 | 0.10 | 166 | 0.227 | 9,872 | 3,498 |
| | | (pH 5.2) | | | | | | |
| | 29 | Citrate | 4 | 0.05 | 65 | 0.404 | 418,644 | 95,190 |
| | | (pH 3.0) | | | | | | |
| | 29 | Acetate | 4 | 0.05 | 84 | 0.285 | 546,335 | 32,895 |
| | | (pH 4.0) | | | | | | |
| | 29 | Acetate | 4 | 0.10 | 233 | 0.323 | 243,618 | 17,531 |
| | | (pH 5.2) | | | | | | |
| | 47 | Citrate | 4 | 0.05 | 82 | 0.443 | 279 | 188 |
| | | (pH 3.0) | | | | | | |
| | 47 | Acetate | 4 | 0.05 | 81 | 0.416 | 775 | 276 |
| | | (pH 4.0) | | | | | | |
| | 47 | Acetate | 4 | 0.10 | 121 | 0.236 | 702 | 109 |
| | | (pH 4.0) | | | | | | |
| | 51 | Citrate | 4 | 0.05 | 89 | 0.361 | 1,176 | 256 |
| | | (pH 3.0) | | | | | | |
| | 51 | Acetate | 4 | 0.05 | 99 | 0.352 | 734 | 62 |
| | | (pH 4.0) | | | | | | |
| | 51 | Acetate | 4 | 0.10 | 115 | 0.254 | 2,498 | 211 |
| | | (pH 4.0) | | | | | | |
| | 52 | Citrate | 4 | 0.05 | 112 | 0.476 | 102 | 35 |
| | | (pH 3.0) | | | | | | |
| | 52 | Acetate | 4 | 0.05 | 175 | 0.754 | 56 | 25 |
| | | (pH 4.0) | | | | | | |
| | 52 | Acetate | 4 | 0.10 | 323 | 0.885 | 38 | 26 |
| | | (pH 4.0) | | | | | | |
| | 43 | Citrate | 4 | 0.05 | 192 | 0.283 | 819 | 548 |
| | | (pH 3.0) | | | | | | |
| | 43 | Acetate | 4 | 0.05 | 106 | 0.493 | 3,876 | 1,046 |
| | | (pH 4.0) | | | | | | |
| | 43 | Acetate | 4 | 0.10 | 199 | 0.311 | 18,268 | 1,889 |
| | | (pH 5.2) | | | | | | |
| | 48 | Citrate | 4 | 0.05 | 187 | 0.464 | 1,831 | 449 |
| | | (pH 3.0) | | | | | | |
| | 48 | Acetate | 4 | 0.05 | 94 | 0.445 | 2,056 | 1,200 |
| | | (pH 4.0) | | | | | | |
| | 48 | Acetate | 4 | 0.10 | 110 | 0.248 | 1,323 | 640 |
| | | (pH 4.0) | | | | | | |
| | 32 | Citrate | 4 | 0.05 | 39 | 0.435 | 51 | 17 |
| | | (pH 3.0) | | | | | | |
| | 32 | Acetate | 4 | 0.05 | 54 | 0.423 | 55 | 14 |
| | | (pH 4.0) | | | | | | |
| | 32 | Acetate | 4 | 0.10 | 61 | 0.245 | 35 | 18 |
| | | (pH 5.2) | | | | | | |

Dimensions of DNPs Containing Luc-mRNA Obtained by Co-assembly of IAJD9 or 22 with Different Concentrations and Luc-mRNA in Acetate Buffer (10 mM) and Luminescence Expression in HEK293T Cells (125 ng/well) are presented in Table 5.

TABLE 5

| | pH of | | | | | | |
|---|---|---|---|---|---|---|---|
| | acetate | | | | | | |
| | buffer | C_{IAJD} | C_{Luc-mRNA} | Size | | | Standard |
| IAJD | (10 mM) | (mg/mL) | (mg/mL) | (d, nm) | PDI | Luminescence | deviation |
| 9 | 4.0 | 8.00 | 0.05 | 66 | 0.281 | 2,929,485 | 200,696 |
| | 4.0 | 6.00 | 0.05 | 65 | 0.295 | 4,732,744 | 455,960 |
| | 4.0 | 5.00 | 0.05 | 44 | 0.341 | 5,357,139 | 189,684 |
| | 4.0 | 4.00 | 0.05 | 59 | 0.291 | 5,316,758 | 140,302 |
| | 4.0 | 3.00 | 0.05 | 66 | 0.282 | 4,150,126 | 954,384 |
| | 4.0 | 2.00 | 0.05 | 65 | 0.258 | 2,533,441 | 484,087 |
| | 4.0 | 1.00 | 0.05 | 71 | 0.270 | 1,566,729 | 370,057 |
| | 4.0 | 0.75 | 0.05 | 75 | 0.241 | 683,904 | 155,907 |
| | 4.0 | 0.50 | 0.05 | 91 | 0.270 | 197,838 | 47,356 |
| | 4.0 | 0.25 | 0.05 | 112 | 0.243 | 32,208 | 8,832 |
| 9 | 4.0 | 4.00 | 0.05 | 68 | 0.153 | 4,832,988 | 612,805 |
| | 4.4 | 4.00 | 0.05 | 50 | 0.280 | 4,949,850 | 601,419 |
| | 4.8 | 4.00 | 0.05 | 56 | 0.281 | 5,278,352 | 476,945 |
| | 5.2 | 4.00 | 0.05 | 74 | 0.259 | 5,306,972 | 421,485 |
| | 5.6 | 4.00 | 0.05 | 140 | 0.209 | 4,053,924 | 307,161 |
| 22 | 4.0 | 8.00 | 0.05 | 50 | 0.302 | 5,246,616 | 131,335 |
| | 4.0 | 6.00 | 0.05 | 45 | 0.292 | 6,493,891 | 108,856 |
| | 4.0 | 5.00 | 0.05 | 52 | 0.272 | 7,317,328 | 192,067 |
| | 4.0 | 4.00 | 0.05 | 53 | 0.288 | 6,922,629 | 427,518 |
| | 4.0 | 3.00 | 0.05 | 57 | 0.291 | 5,695,413 | 221,600 |
| | 4.0 | 2.00 | 0.05 | 55 | 0.256 | 5,269,341 | 508,483 |
| | 4.0 | 1.00 | 0.05 | 65 | 0.252 | 2,502,511 | 388,969 |
| | 4.0 | 0.75 | 0.05 | 72 | 0.232 | 891,902 | 191,482 |
| | 4.0 | 0.50 | 0.05 | 71 | 0.195 | 178,965 | 53,060 |
| | 4.0 | 0.25 | 0.05 | 92 | 0.204 | 41,897 | 5,500 |
| 22 | 4.0 | 4.00 | 0.05 | 49 | 0.307 | 5,069,642 | 275,369 |
| | 4.4 | 4.00 | 0.05 | 66 | 0.288 | 5,409,310 | 427,776 |
| | 4.8 | 4.00 | 0.05 | 74 | 0.264 | 5,106,741 | 405,158 |
| | 5.2 | 4.00 | 0.05 | 71 | 0.237 | 5,390,693 | 402,291 |
| | 5.6 | 4.00 | 0.05 | 115 | 0.225 | 3,939,728 | 668,494 |

Dimensions of DNPs Containing Luc-mRNA Obtained by Co-assembly of IAJD33 or 34 with Different Concentrations and Luc-mRNA in Acetate Buffer (10 mM) and Luminescence Expression in HEK293T Cells (125 ng/well) are presented in Table 6.

TABLE 6

| | pH of | | | | | | |
|---|---|---|---|---|---|---|---|
| | acetate | | | | | | |
| | buffer | C_{IAJD} | C_{Luc-mRNA} | Size | | | Standard |
| IAJD | (10 mM) | (mg/mL) | (mg/mL) | (d, nm) | PDI | Luminescence | deviation |
| 33 | 4.0 | 8.00 | 0.05 | 94 | 0.262 | 4,707 | 870 |
| | 4.0 | 7.00 | 0.05 | 85 | 0.283 | 7,539 | 1,049 |
| | 4.0 | 6.00 | 0.05 | 72 | 0.277 | 15,845 | 2,552 |
| | 4.0 | 5.00 | 0.05 | 75 | 0.263 | 23,237 | 8,898 |
| | 4.0 | 4.00 | 0.05 | 66 | 0.256 | 32,404 | 24,064 |
| | 4.0 | 3.00 | 0.05 | 60 | 0.274 | 62,770 | 14,762 |
| | 4.0 | 2.00 | 0.05 | 59 | 0.263 | 74,283 | 11,774 |
| | 4.0 | 1.00 | 0.05 | 64 | 0.207 | 56,565 | 18,157 |
| | 4.0 | 0.75 | 0.05 | 74 | 0.221 | 44,198 | 23,163 |
| | 4.0 | 0.50 | 0.05 | 74 | 0.209 | 19,310 | 1,217 |
| | 4.0 | 0.25 | 0.05 | 88 | 0.198 | 14,035 | 3,681 |
| 34 | 4.0 | 8.00 | 0.05 | 85 | 0.245 | 4,362 | 2,487 |
| | 4.0 | 7.00 | 0.05 | 69 | 0.289 | 9,060 | 3,341 |
| | 4.0 | 6.00 | 0.05 | 59 | 0.238 | 15,583 | 7,266 |
| | 4.0 | 5.00 | 0.05 | 59 | 0.256 | 31,303 | 7,940 |
| | 4.0 | 4.00 | 0.05 | 56 | 0.255 | 52,554 | 36,103 |
| | 4.0 | 3.00 | 0.05 | 50 | 0.212 | 51,933 | 17,038 |
| | 4.0 | 2.00 | 0.05 | 49 | 0.212 | 90,071 | 59,289 |
| | 4.0 | 1.00 | 0.05 | 64 | 0.206 | 95,041 | 56,801 |
| | 4.0 | 0.75 | 0.05 | 73 | 0.231 | 46,628 | 37,069 |
| | 4.0 | 0.50 | 0.05 | 65 | 0.209 | 5,491 | 2,269 |
| | 4.0 | 0.25 | 0.05 | 88 | 0.211 | 61 | 26 |

Dimensions of DNPs Containing Luc-mRNA and Luminescence Expression in vivo are presented in Table 7.

TABLE 7

| | | | | pH | | | | | |
|---|---|---|---|---|---|---|---|---|---|
| | | C_{IAJD} | C_{Luc-mRNA} | Acetate | | Size | | T | Organs with |
| IAJD | | (mg/mL) | (mg/mL) | buffer | Sample | (d, nm) | PDI | (h)^{a} | luminescence |
| Library 1 | 1 | 4.0 | 0.1 | 5.2 | 7.10 | 295 | 0.501 | 6 | Lung, spleen |
| Single- | 2 | 4.0 | 0.1 | 5.2 | 7.13 | 289 | 0.521 | 6 | Lung, spleen |
| Single | 3 | 4.0 | 0.1 | 5.2 | 7.07 | 95 | 0.244 | 7 | — |
| | 4 | 4.0 | 0.1 | 5.2 | 7.45 | 178 | 0.284 | 7 | — |
| | 5 | 4.0 | 0.1 | 5.2 | 7.78 | 156 | 0.281 | 7 | — |
| | 6 | 4.0 | 0.1 | 5.2 | 6.30 | 92 | 0.246 | — | — |
| | 7 | 4.0 | 0.1 | 5.2 | 6.08 | 132 | 0.338 | — | — |
| | 8 | 4.0 | 0.1 | 5.2 | 7.23 | 142 | 0.269 | 7 | Lung |
| | 9a | 4.0 | 0.1 | 5.2 | 7.30 | 98 | 0.224 | 6 | Lung |
| | 9b | 4.0 | 0.025 | 3.0^{b} | 3.71 | 49 | 0.464 | 5 | Lung, liver, |
| | | | | | | | | | spleen |
| Library 2 | 19 | 4.0 | 0.1 | 5.2 | 7.52 | 110 | 0.167 | 6 | Lung, liver, |
| Single- | | | | | | | | | spleen |
| Single | 20 | 4.0 | 0.1 | 5.2 | 7.63 | 120 | 0.203 | 6 | Lung, spleen |
| | 21 | 4.0 | 0.1 | 5.2 | 7.23 | 102 | 0.200 | 6 | — |
| | 22 | 4.0 | 0.1 | 5.2 | 7.39 | 114 | 0.232 | 6 | Lung |
| | 23 | 4.0 | 0.1 | 5.2 | 7.74 | 108 | 0.230 | 6 | Lung, liver, |
| | | | | | | | | | spleen |
| | 24 | 4.0 | 0.1 | 5.2 | 7.61 | 108 | 0.216 | 6 | Lung, spleen |
| | 25 | 4.0 | 0.1 | 5.2 | 6.90 | 147 | 0.202 | 6 | Lung, spleen |
| Library 3 | 44 | 4.0 | 0.1 | 5.2 | 7.15 | 171 | 0.252 | 4 | Lung, liver |
| Single- | 33 | 4.0 | 0.1 | 4.0 | 4.96 | 73 | 0.248 | 6 | Lung, liver |
| Single | 34 | 4.0 | 0.1 | 4.0 | 5.10 | 83 | 0.245 | 6 | Lung, liver |
| | 45 | 4.0 | 0.1 | 5.2 | 6.87 | 512 | 0.536 | 4 | Lung |
| | 35 | 4.0 | 0.1 | 4.0 | 4.76 | 70 | 0.243 | 6 | — |
| | 36 | 4.0 | 0.1 | 4.0 | 4.90 | 71 | 0.224 | 6 | — |
| Library 4 | 30a | 4.0 | 0.1 | 5.2 | 5.95 | 165 | 0.377 | 7 | Lung, liver, |
| Single- | | | | | | | | | spleen |
| Single | 30b | 4.0 | 0.1 | 5.2 | 6.13 | 134 | 0.408 | 7 | Lung, liver, |
| | | | | | | | | | spleen |
| | 31 | 4.0 | 0.1 | 5.2 | 7.02 | 210 | 0.261 | 7 | Lung, liver, |
| | | | | | | | | | spleen |
| | 37 | 4.0 | 0.1 | 4.0 | 4.52 | 131 | 0.267 | 6 | Lung, liver, |
| | | | | | | | | | spleen |
| Library 5 | 10 | 4.0 | 0.1 | 5.2 | 7.76 | 347 | 0.449 | 7 | Lung |
| Twin-Twin | 11 | 4.0 | 0.1 | 5.2 | 7.13 | 369 | 0.477 | 7 | — |
| | 12 | 4.0 | 0.1 | 5.2 | 7.35 | 171 | 0.467 | 7 | — |
| | 13 | 4.0 | 0.1 | 5.2 | 7.35 | 163 | 0.299 | 7 | — |
| | 14 | 4.0 | 0.1 | 5.2 | 7.26 | 98 | 0.206 | 6 | — |
| | 15 | 4.0 | 0.1 | 5.2 | 5.78 | 103 | 0.262 | — | — |
| | 16 | 4.0 | 0.1 | 5.2 | 5.84 | 108 | 0.183 | — | — |
| | 17 | 4.0 | 0.1 | 5.2 | 7.13 | 203 | 0.449 | 6 | Lung, spleen |
| | 18 | 4.0 | 0.1 | 5.2 | 7.17 | 156 | 0.370 | 6 | Lung, spleen |
| | 46 | 4.0 | 0.1 | 4.0 | 4.68 | 111 | 0.217 | 4 | Lung, liver, |
| | | | | | | | | | spleen |
| Library 6 | 38 | 4.0 | 0.1 | 5.2 | 6.59 | 418 | 0.364 | 4 | — |
| Hybrid | 39 | 4.0 | 0.1 | 5.2 | 6.66 | 242 | 0.392 | 4 | — |
| Twin-Mix | 40 | 4.0 | 0.1 | 5.2 | 6.87 | 423 | 0.388 | 4 | Lung |
| | 41 | 4.0 | 0.1 | 5.2 | 7.68 | 254 | 0.517 | 4 | — |
| | 42 | 4.0 | 0.1 | 5.2 | 7.26 | 307 | 0.570 | 4 | — |
| | 49 | 4.0 | 0.1 | 5.2 | 7.27 | 242 | 0.340 | 4 | — |
| | 26 | 4.0 | 0.1 | 5.2 | 6.93 | 227 | 0.448 | 6 | Lung, liver, |
| | | | | | | | | | spleen |
| | 27 | 4.0 | 0.1 | 5.2 | 6.83 | 306 | 0.538 | 6 | Lung, liver, |
| | | | | | | | | | spleen |
| | 28 | 4.0 | 0.1 | 5.2 | 6.67 | 266 | 0.488 | 7 | Lung, liver |
| | 50 | 4.0 | 0.1 | 5.2 | 7.53 | 166 | 0.227 | 4 | — |
| | 29 | 4.0 | 0.1 | 5.2 | 6.81 | 233 | 0.323 | 7 | Lung, liver, |
| | | | | | | | | | spleen |
| | 47 | 4.0 | 0.1 | 4.0 | 4.29 | 121 | 0.236 | 4 | Lung |
| | 51 | 4.0 | 0.1 | 4.0 | 4.89 | 115 | 0.254 | 6 | — |
| | 52 | 4.0 | 0.1 | 4.0 | 4.84 | 323 | 0.885 | 6 | — |
| | 43 | 4.0 | 0.1 | 5.2 | 7.15 | 199 | 0.311 | 4 | Lung, spleen |
| | 48 | 4.0 | 0.1 | 4.0 | 4.34 | 110 | 0.248 | — | — |
| | 32 | 4.0 | 0.1 | 5.2 | 6.44 | 61 | 0.245 | 6 | — |

^{a}Time from injection to the characterization of mice.
^{b}Sample was prepared in citrate buffer (10 mM, pH = 3.0) and then dialyzed against 1X PBS for 2 h at 4° C.

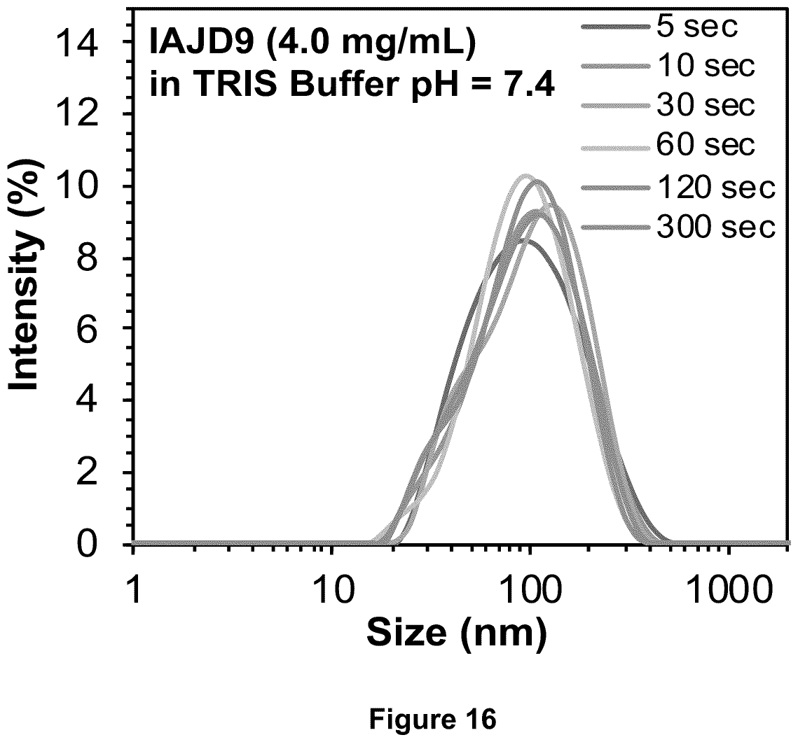

The effect of vortex time on the dimensions of assemblies of IAJDS was determined. The results are shown in Table 8 and in FIG. 16.

TABLE 8

| Sample | Vortex Time | Size | |
|---|---|---|---|
| No. | (Sec) | (nm) | PDI |
| 1 | 5 | 81 | 0.250 |
| 2 | 10 | 81 | 0.251 |
| 3 | 30 | 85 | 0.240 |
| 4 | 60 | 81 | 0.247 |
| 5 | 120 | 78 | 0.250 |
| 6 | 300 | 77 | 0.249 |

Sample preparation: Injected 25 μL of IAJD9 (80 mg/mL) ethanol stock solution into 475 μL tris buffer (pH 7.4), followed by vortex for (5-300) seconds. The final concentration of IAJD9 was 4.0 mg/mL.

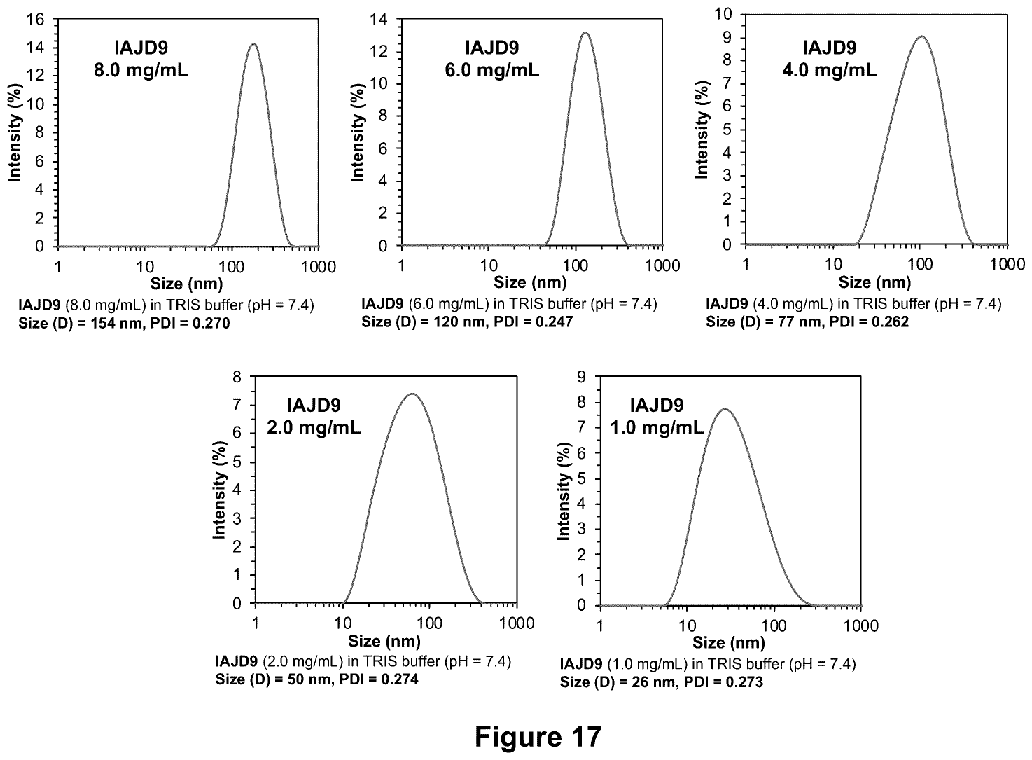

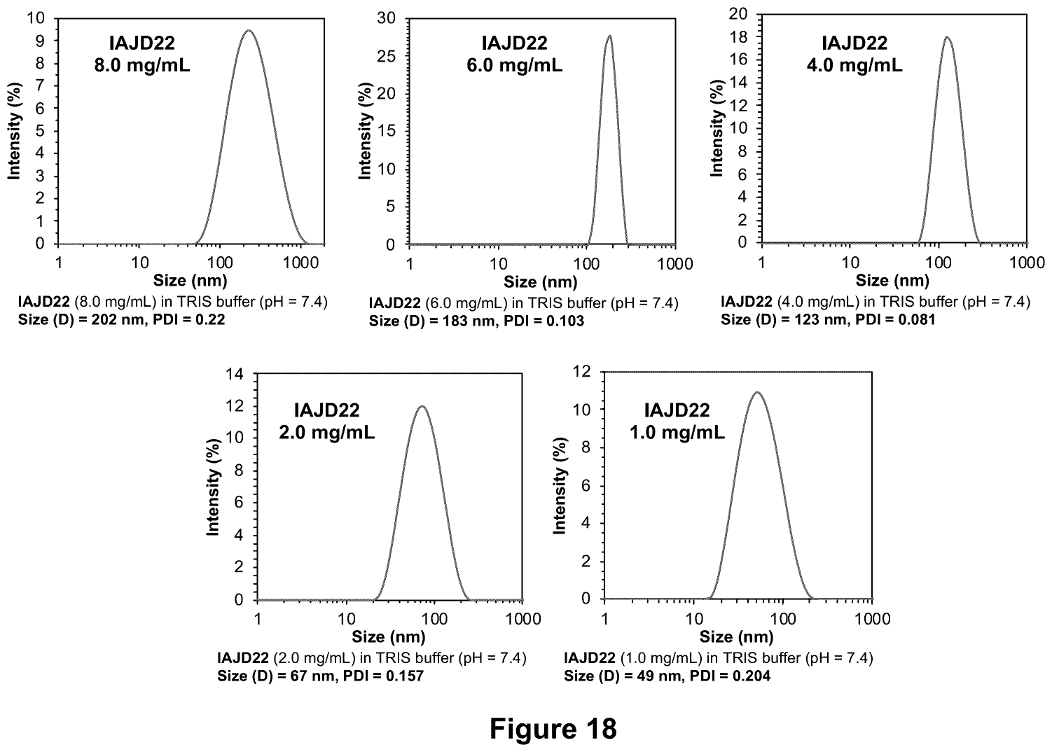

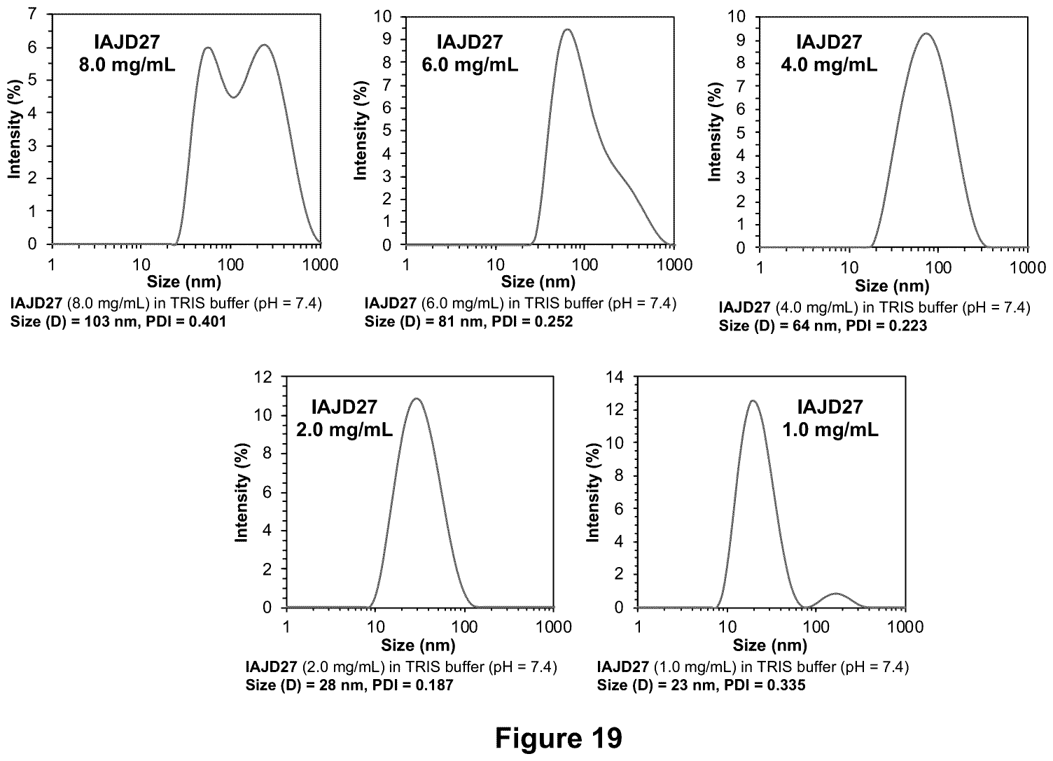

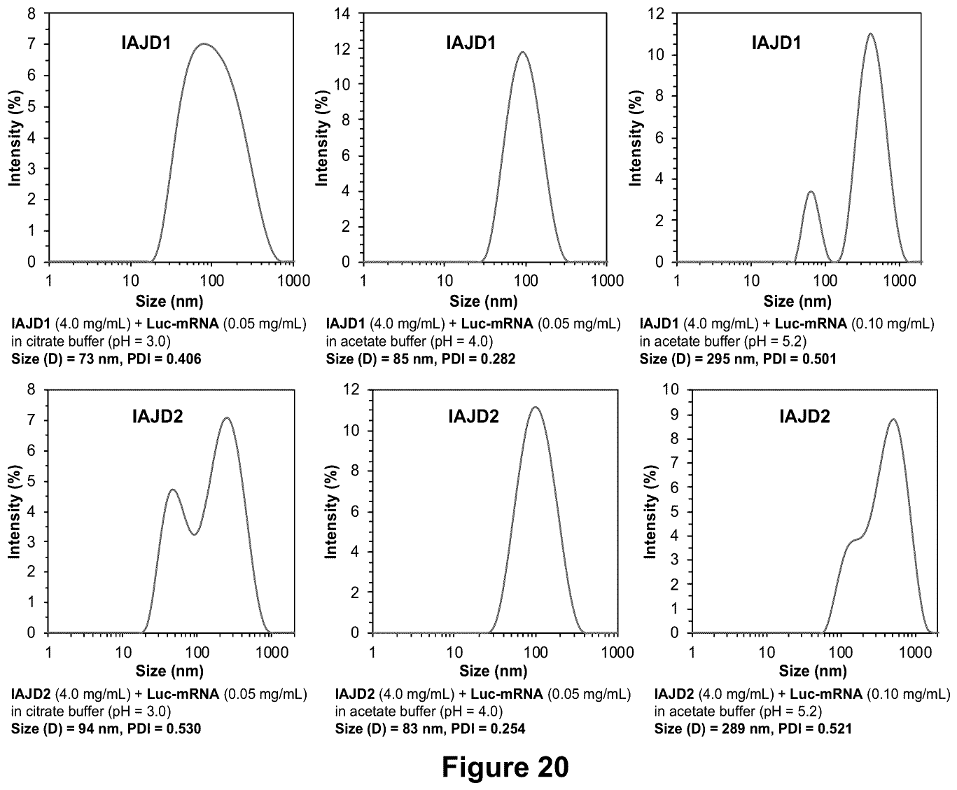

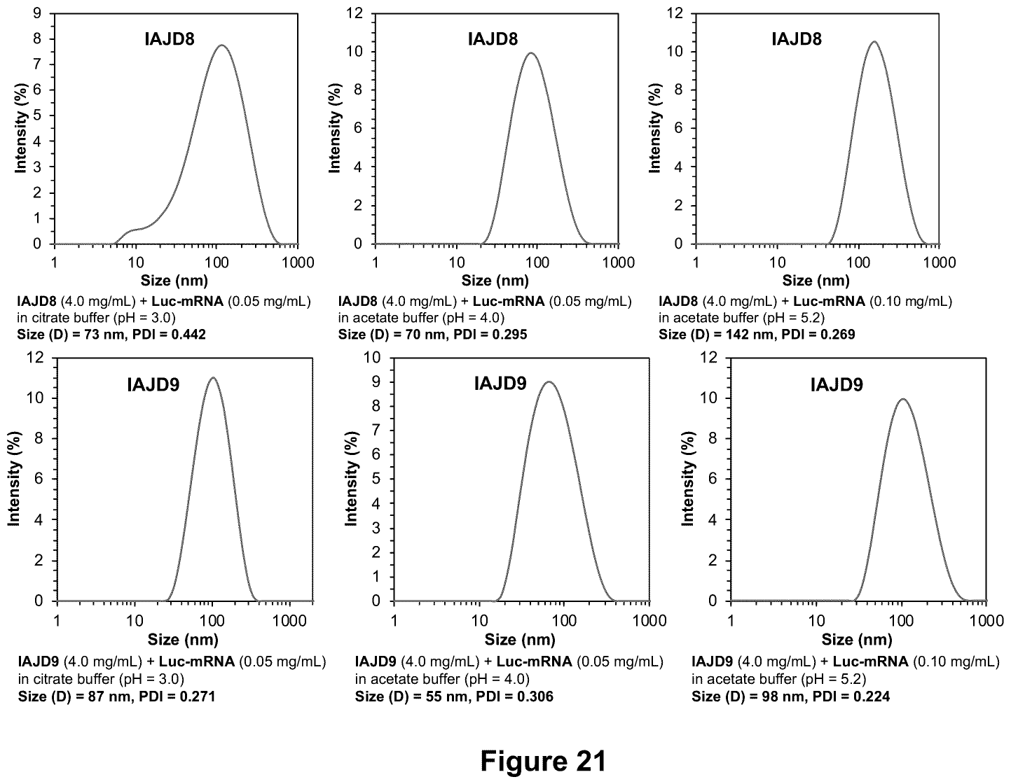

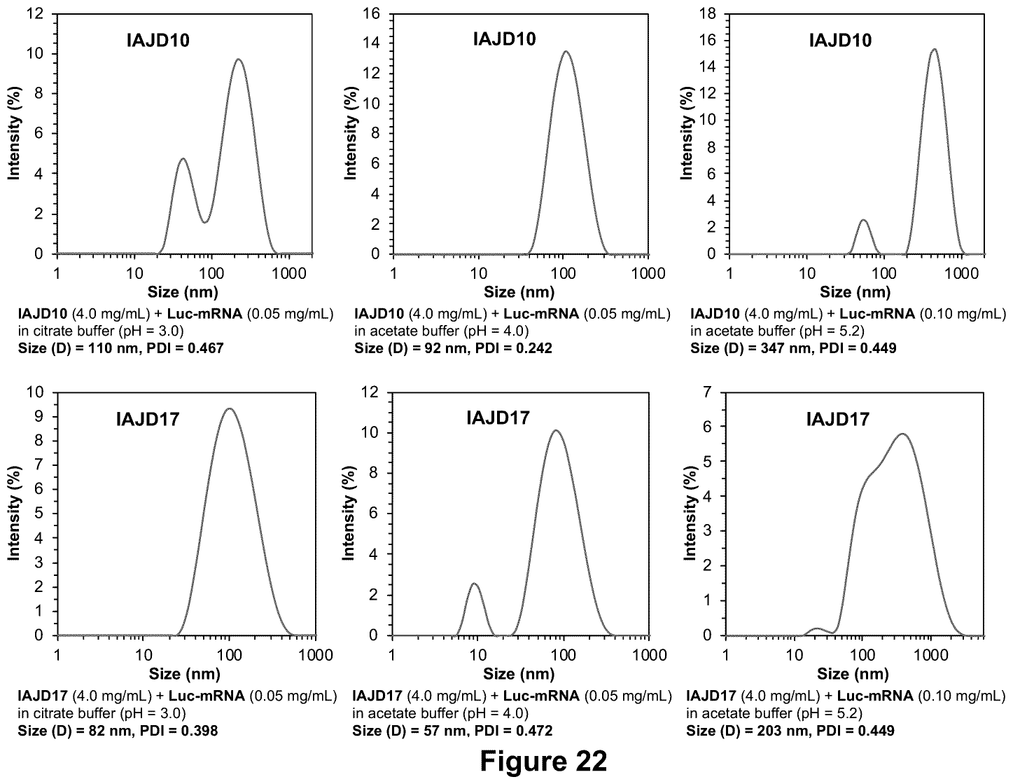

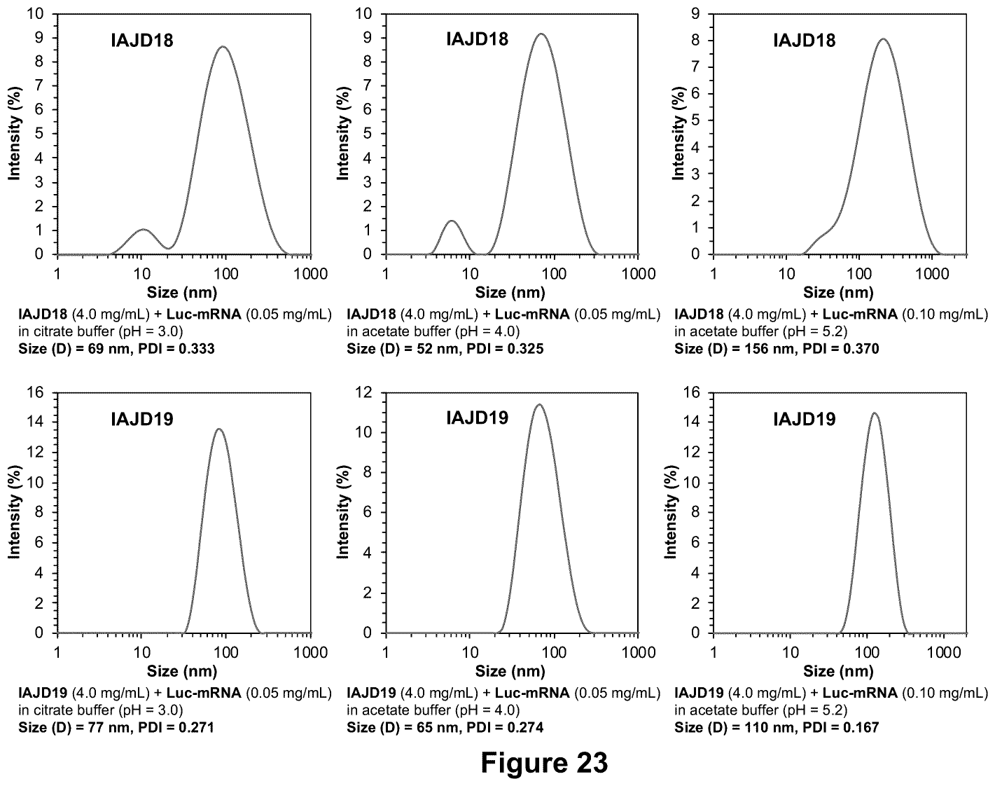

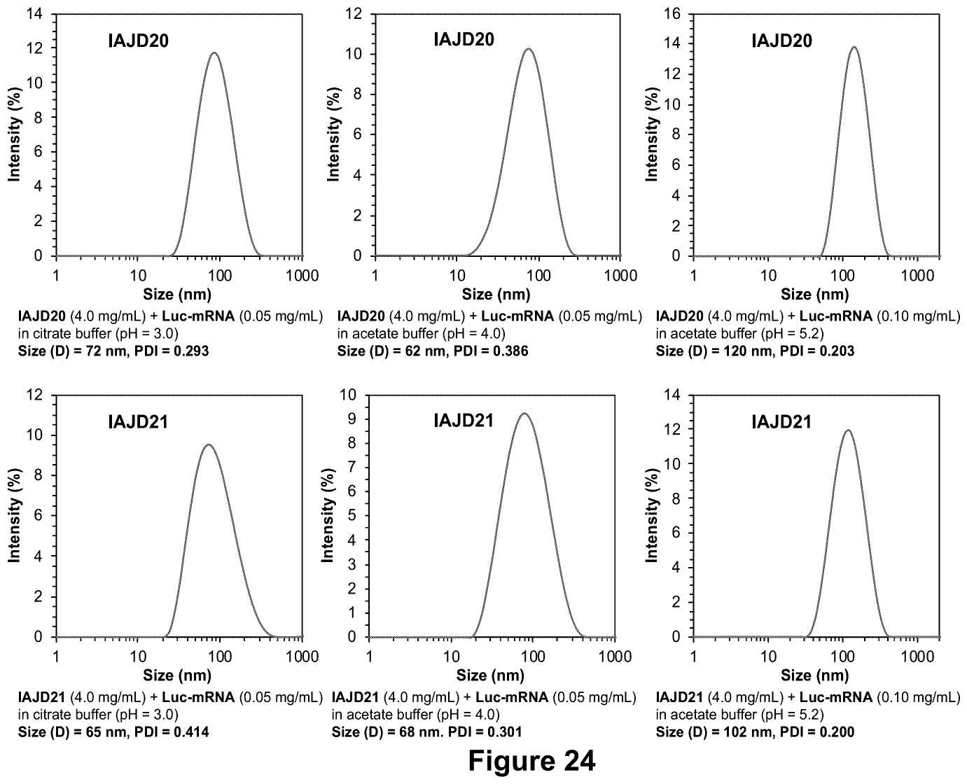

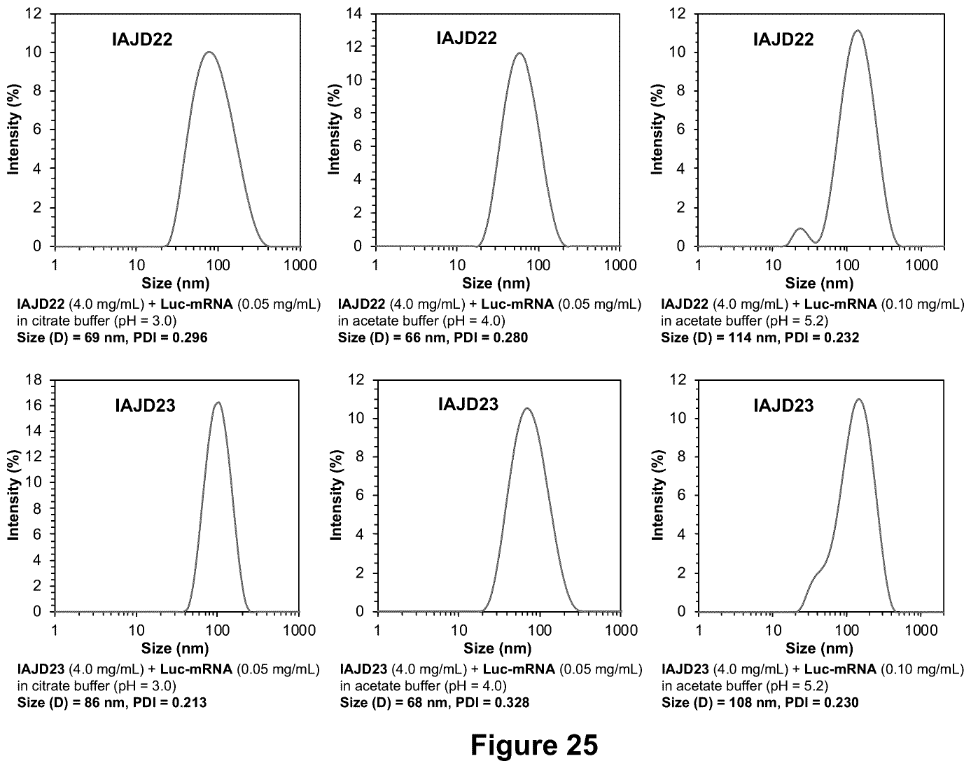

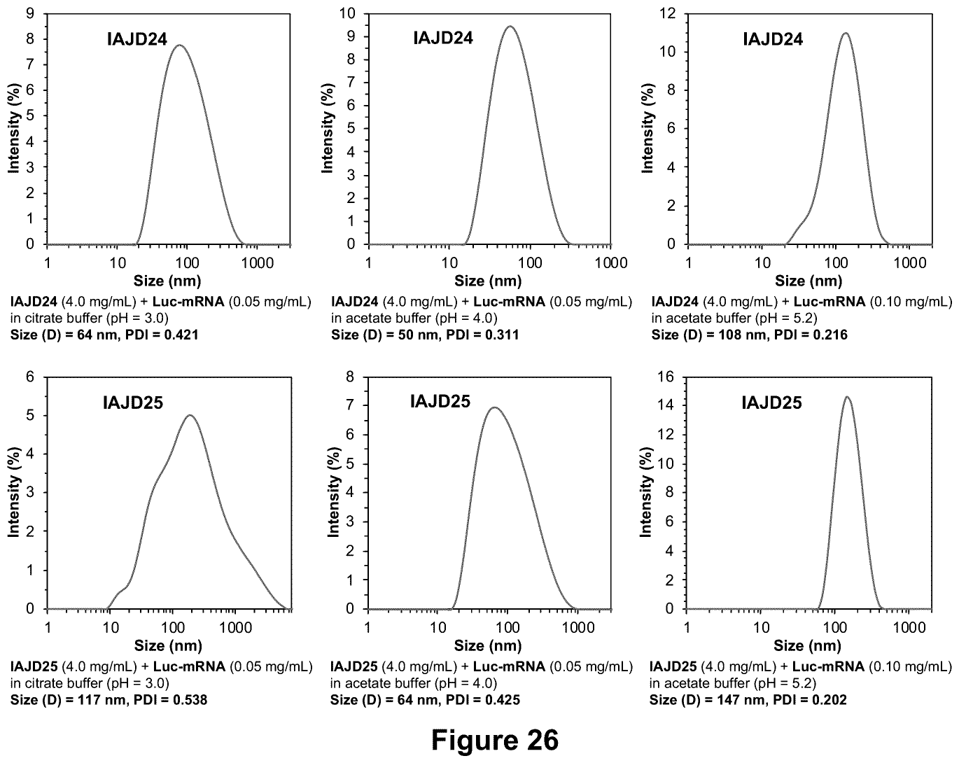

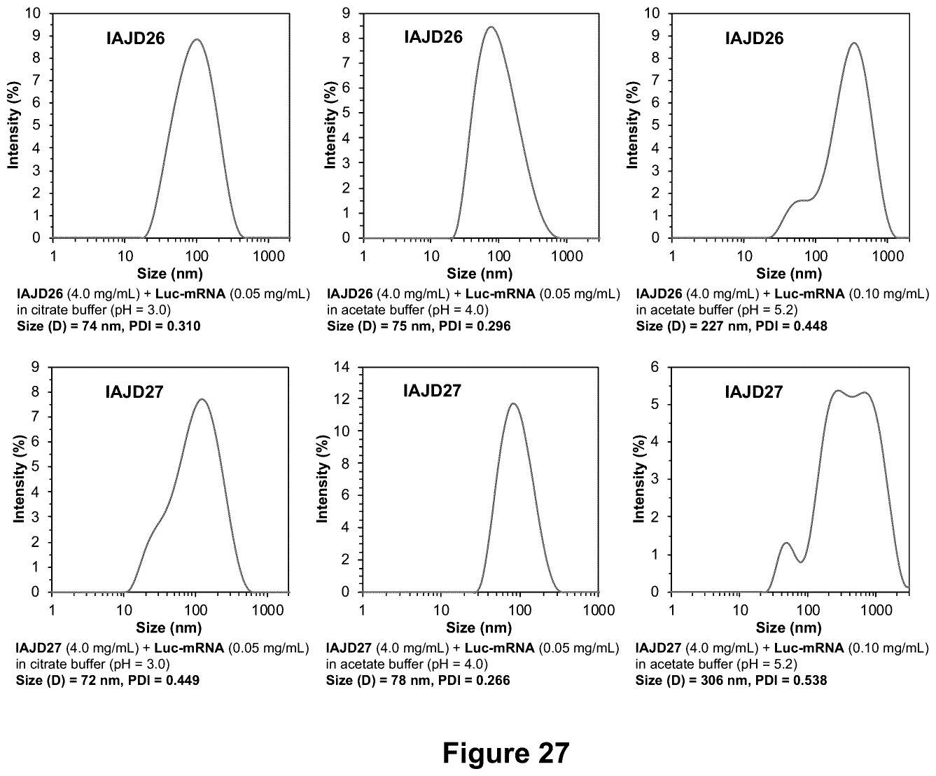

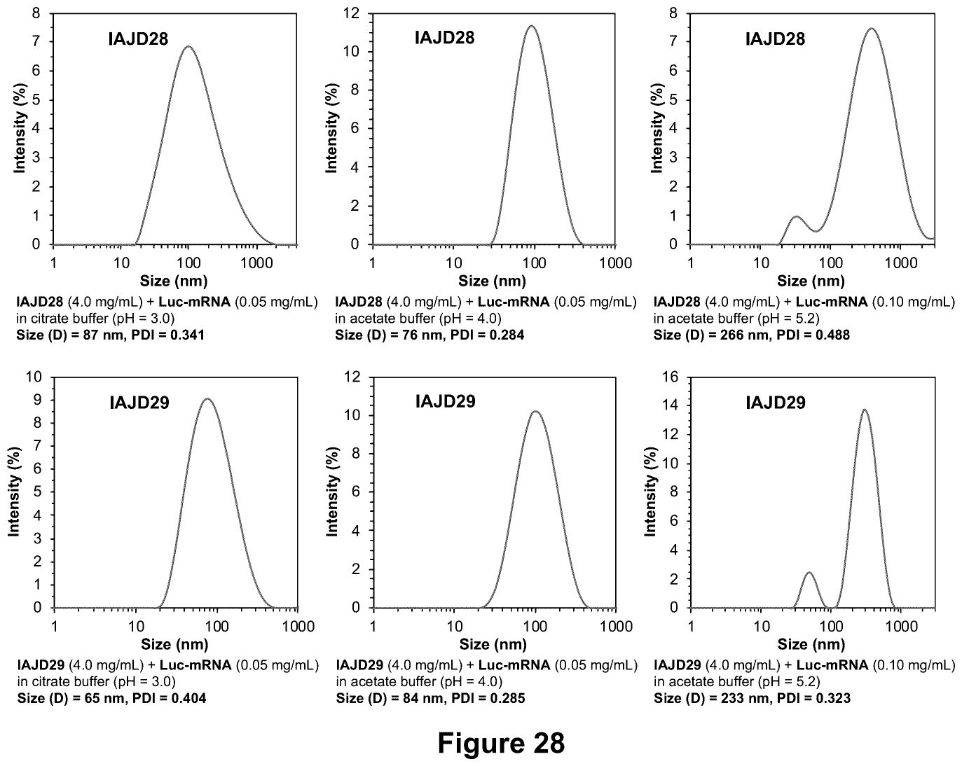

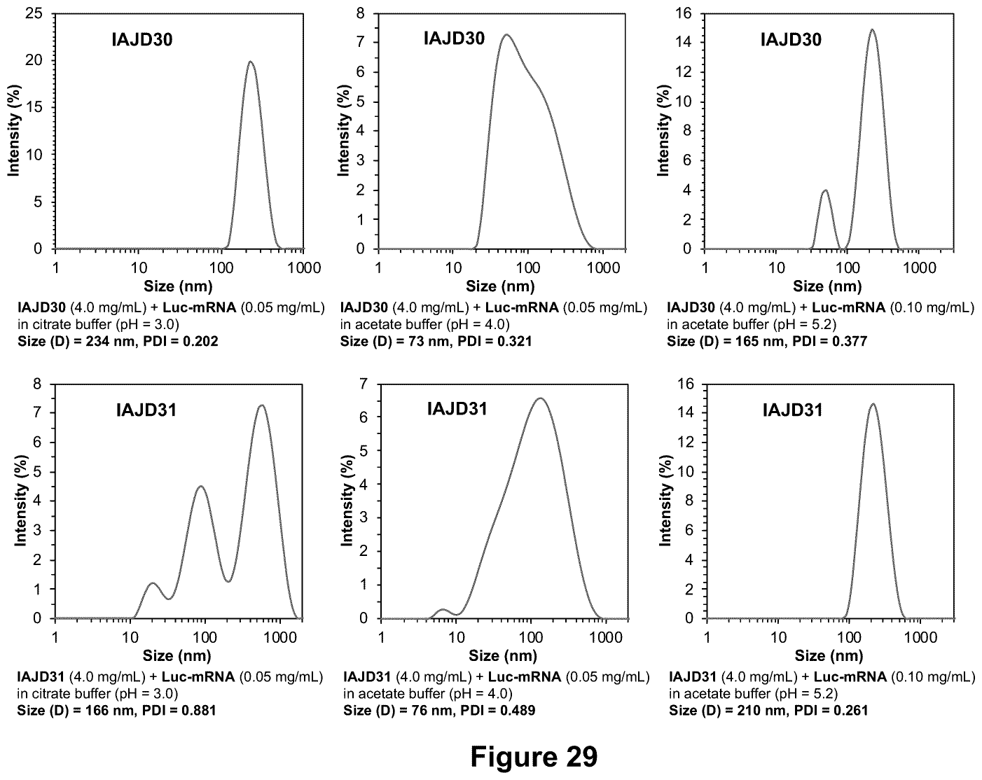

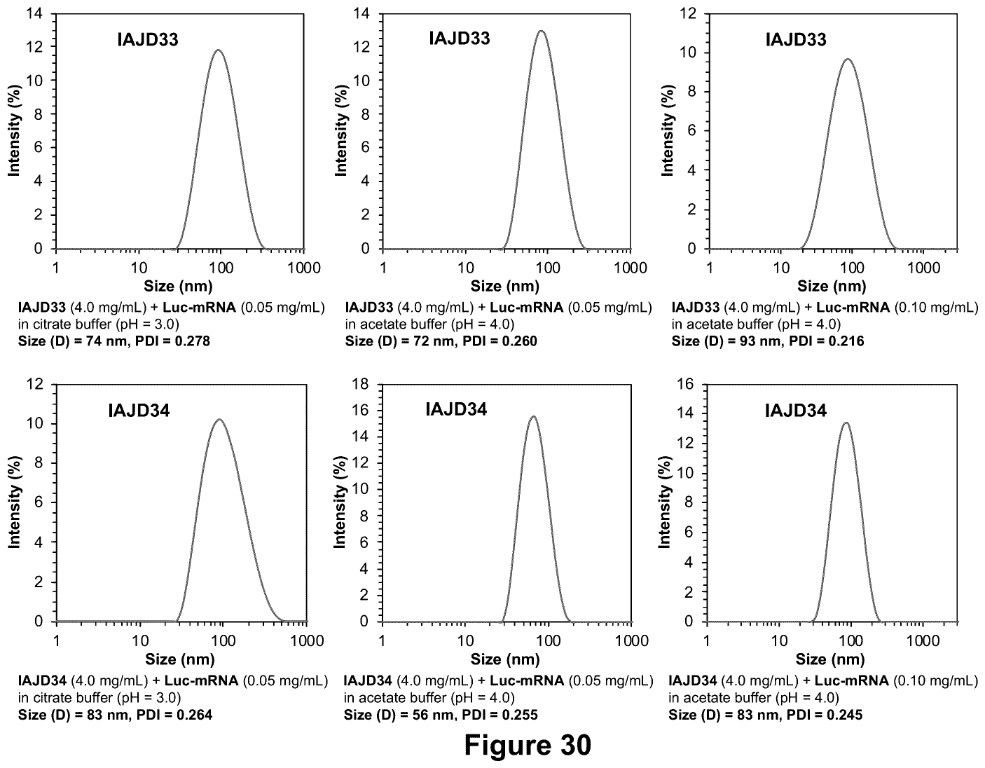

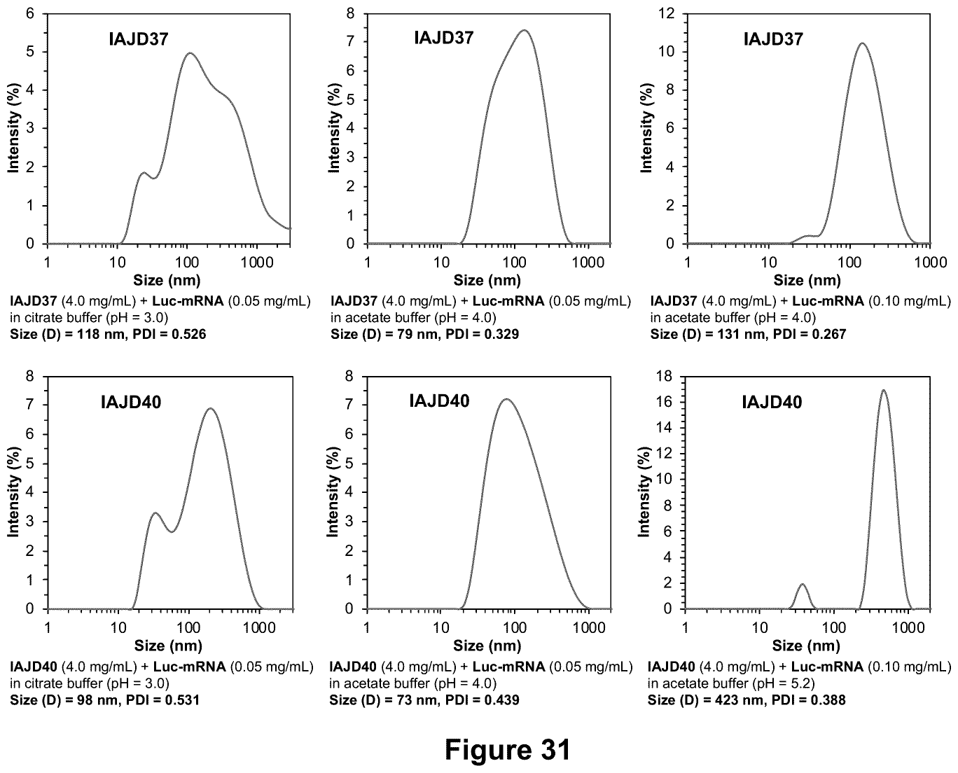

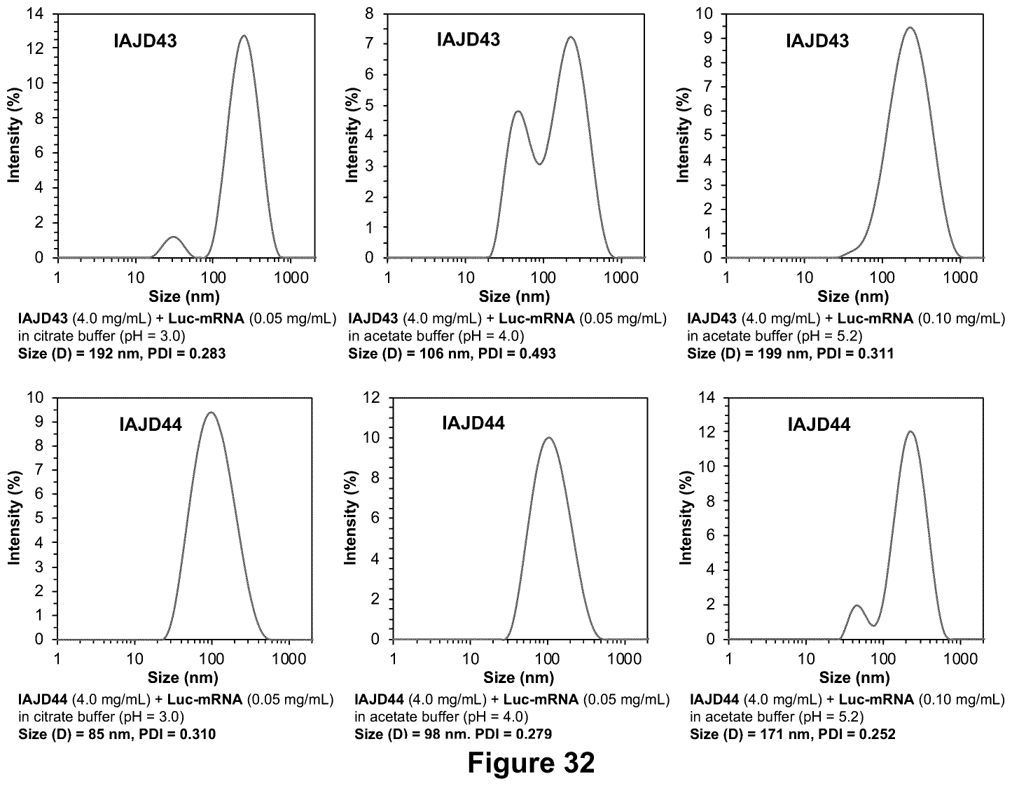

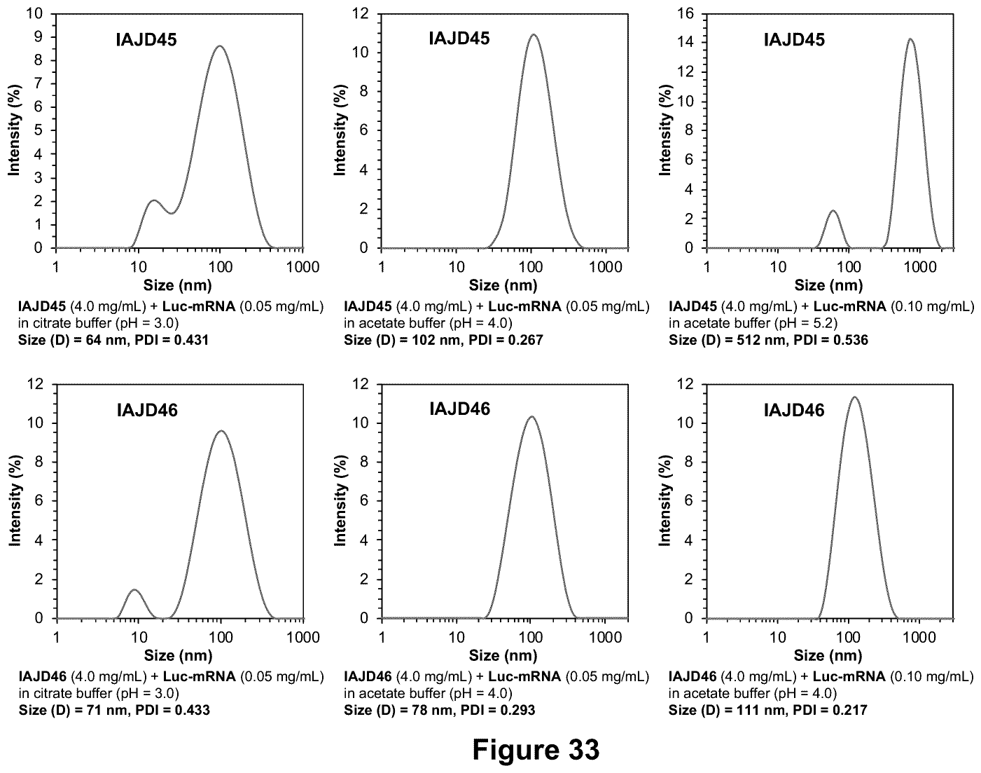

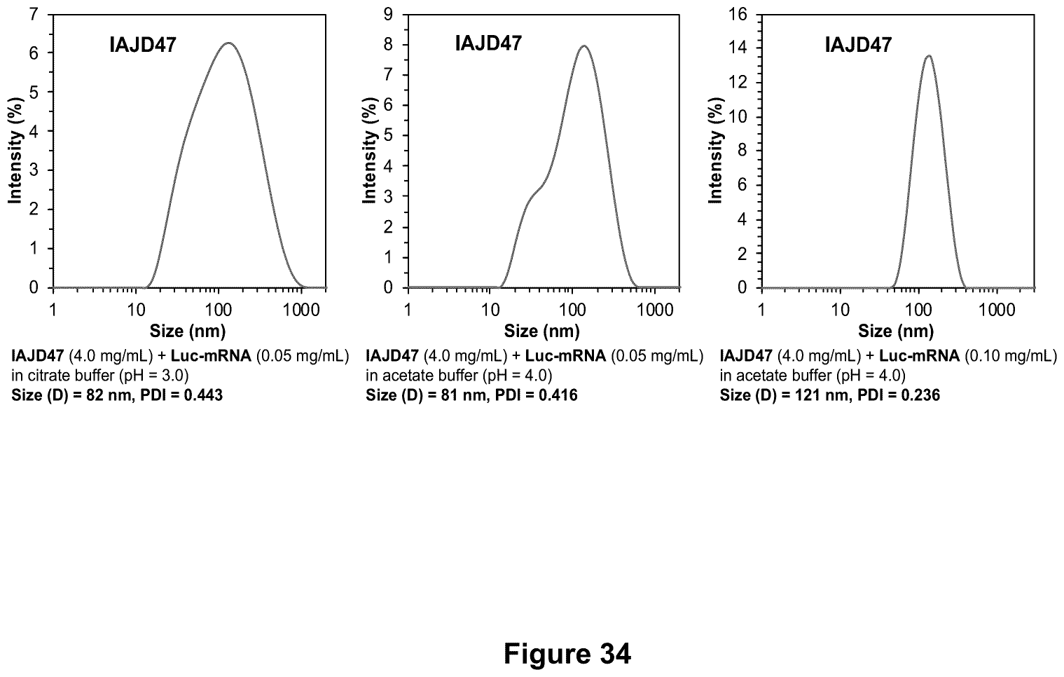

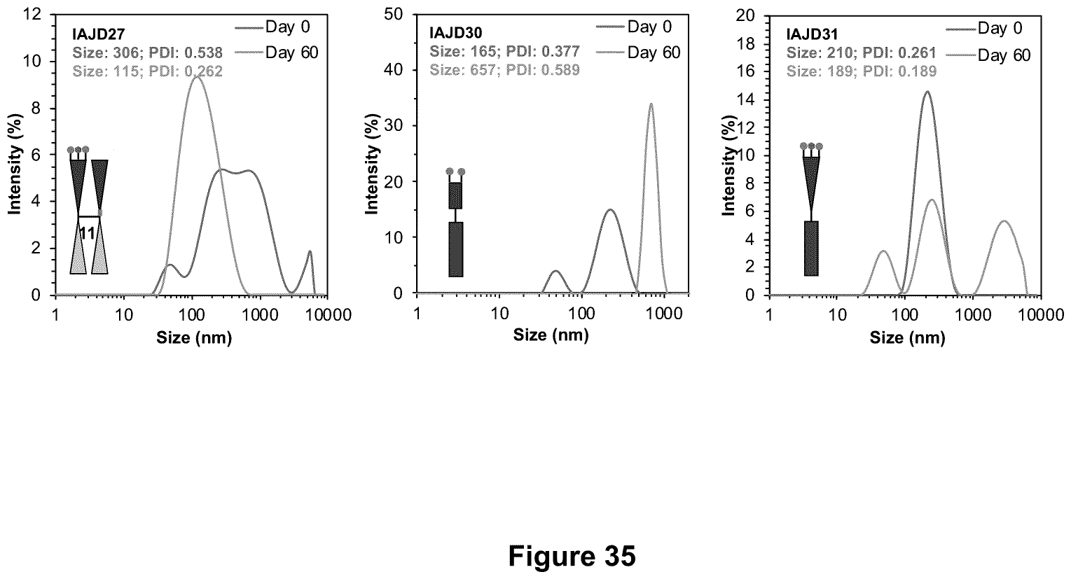

The DLS data of assemblies of the dendrimers is presented in FIG. 17 through FIG. 35.

The stability of the DNPs as assayed by DLS. All the samples are prepared in acetate buffer. The stabilities of the constructs were ranked: Excellent (in bold)—the original sizes and distribution remains almost the same after storage; Good—the original relatively big sizes became smaller or broad size distribution became narrower; Not stable—the original relatively big sizes or broad size distribution remained almost the similar level, or the original relatively small sizes became bigger or narrow size distribution became broader. The results are shown in Table 9 Examples of excellent stability are presented in FIG. 11.

TABLE 9
Stability of DNPs by DLS.

| | | | | pH | | Original | | | | |
|---|---|---|---|---|---|---|---|---|---|---|
| IAJD | | C_{IAJD} | C_{Luc-mRNA} | Acetate | | size | | Size | | Ranking of |
| (storage time) | | (mg/mL) | (mg/mL) | buffer | Sample | (d, nm) | PDI | (d, nm) | PDI | stability^{c} |
| Library 1 | 1 (60 | 4.0 | 0.1 | 5.2 | 7.10 | 295 | 0.501 | 215 | 1.000 | Not stable |
| Single- | days) | | | | | (M)^{b} | | (M) | | |
| Single | 2 (60 | 4.0 | 0.1 | 5.2 | 7.13 | 289 | 0.521 | 164 | 1.000 | Not stable |
| | days) | | | | | (M) | | (M) | | |
| | 3 (60 | 4.0 | 0.1 | 5.2 | 7.07 | 95 | 0.244 | 1,666 | 0.733 | Not stable |
| | days) | | | | | (U) | | (B) | | |
| | 4 (60 | 4.0 | 0.1 | 5.2 | 7.45 | 178 | 0.284 | 4,923 | 0.155 | Not stable |
| | days) | | | | | (B) | | (U) | | |
| | 5 (60 | 4.0 | 0.1 | 5.2 | 7.78 | 156 | 0.281 | 302 | 0.509 | Not stable |
| | days) | | | | | (U) | | (B) | | |
| | 6 (60 | 4.0 | 0.1 | 5.2 | 6.30 | 92 | 0.246 | 251 | 0.464 | Not stable |
| | days) | | | | | (B) | | (U) | | |
| | 7 (60 | 4.0 | 0.1 | 5.2 | 6.08 | 132 | 0.338 | 108 | 0.686 | Not stable |
| | days) | | | | | (B) | | (M) | | |
| | 8 (60 | 4.0 | 0.1 | 5.2 | 7.23 | 142 | 0.269 | 327 | 0.526 | Not stable |
| | days) | | | | | (U) | | (M) | | |
| | 9 (120 | 4.0 | 0.05 | 4.0 | — | 55 | 0.306 | 59 | 0.296 | Excellent |
| | days) | | | | | (U) | | (U) | | |
| Library 2 | 19 (60 | 4.0 | 0.1 | 5.2 | 7.52 | 110 | 0.167 | 141 | 0.118 | Excellent |
| Single- | days) | | | | | (U) | | (U) | | |
| Single | 20 (60 | 4.0 | 0.1 | 5.2 | 7.63 | 120 | 0.203 | 122 | 0.152 | Excellent |
| | days) | | | | | (U) | | (U) | | |
| | 21 (60 | 4.0 | 0.1 | 5.2 | 7.23 | 102 | 0.200 | 933 | 1.000 | Not stable |
| | days) | | | | | (U) | | (B) | | |
| | 22 (120 | 4.0 | 0.05 | 4.0 | — | 66 | 0.280 | 67 | 0.249 | Excellent |
| | days) | | | | | (U) | | (U) | | |
| | 23 (60 | 4.0 | 0.1 | 5.2 | 7.74 | 108 | 0.230 | 117 | 0.123 | Excellent |
| | days) | | | | | (U) | | (U) | | |
| | 24 (60 | 4.0 | 0.1 | 5.2 | 7.61 | 108 | 0.216 | 126 | 0.217 | Excellent |
| | days) | | | | | (U) | | (U) | | |
| | 25 (60 | 4.0 | 0.1 | 5.2 | 6.90 | 147 | 0.202 | 107 | 0.538 | Not stable |
| | days) | | | | | (U) | | (B) | | |
| Library 3 | 44 (23 | 4.0 | 0.1 | 5.2 | 7.15 | 171 | 0.252 | 202 | 0.297 | Excellent |
| Single- | days) | | | | | (B) | | (B) | | |
| Single | 33 (70 | 4.0 | 0.1 | 4.0 | 4.96 | 93 | 0.216 | 96 | 0.225 | Excellent |
| | days) | | | | | (U) | | (U) | | |
| | 34 (70 | 4.0 | 0.1 | 4.0 | 5.10 | 83 | 0.245 | 86 | 0.205 | Excellent |
| | days) | | | | | (U) | | (U) | | |
| | 45 (23 | 4.0 | 0.1 | 5.2 | 6.87 | 512 | 0.536 | 283 | 0.501 | Not stable |
| | days) | | | | | (M) | | (B) | | |
| Library 4 | 30 (60 | 4.0 | 0.1 | 5.2 | 5.95 | 165 | 0.377 | 657 | 0.589 | Not stable |
| Single- | days) | | | | | (B) | | (U) | | |
| Single | 31 (60 | 4.0 | 0.1 | 5.2 | 7.02 | 210 | 0.261 | 189 | 0.910 | Not stable |
| | days) | | | | | (U) | | (M) | | |
| | 37 (50 | 4.0 | 0.1 | 4.0 | 4.52 | 131 | 0.267 | 130 | 0.215 | Excellent |
| | days) | | | | | (U) | | (U) | | |
| Library 5 | 10 (60 | 4.0 | 0.05 | 4.0 | — | 92 | 0.242 | 99 | 0.269 | Excellent |
| Twin-Twin | days) | | | | | (U) | | (U) | | |
| | 14 (60 | 4.0 | 0.1 | 5.2 | 7.26 | 98 | 0.206 | 235 | 0.289 | Not stable |
| | days) | | | | | (U) | | (U) | | |
| | 15 (60 | 4.0 | 0.1 | 5.2 | 5.78 | 103 | 0.262 | 95 | 0.287 | Excellent |
| | days) | | | | | (U) | | (U) | | |
| | 16 (60 | 4.0 | 0.1 | 5.2 | 5.84 | 108 | 0.183 | 103 | 0.218 | Excellent |
| | days) | | | | | (U) | | (U) | | |
| | 17 (60 | 4.0 | 0.1 | 5.2 | 7.13 | 203 | 0.449 | 149 | 0.153 | Good |
| | days) | | | | | (M) | | (U) | | |
| | 18 (60 | 4.0 | 0.1 | 5.2 | 7.17 | 156 | 0.370 | 131 | 0.136 | Good |
| | days) | | | | | (U) | | (U) | | |
| | 46 (21 | 4.0 | 0.1 | 4.0 | 4.68 | 111 | 0.217 | 102 | 0.240 | Excellent |
| | days) | | | | | (U) | | (U) | | |
| Library 6 | 40 (23 | 4.0 | 0.1 | 5.2 | 6.87 | 423 | 0.388 | 302 | 0.158 | Good |
| Hybrid | days) | | | | | (B) | | (U) | | |
| Twin-Mix | 49 (21 | 4.0 | 0.1 | 5.2 | 7.27 | 242 | 0.340 | 193 | 0.179 | Good |
| | days) | | | | | (U) | | (U) | | |
| | 27 (60 | 4.0 | 0.1 | 5.2 | 6.83 | 306 | 0.538 | 115 | 0.262 | Good |
| | days) | | | | | (M) | | (U) | | |
| | 50 (21 | 4.0 | 0.1 | 5.2 | 7.53 | 166 | 0.227 | 165 | 0.229 | Excellent |
| | days) | | | | | (U) | | (U) | | |
| | 29 (60 | 4.0 | 0.1 | 5.2 | 6.81 | 233 | 0.323 | 151 | 0.256 | Good |
| | days) | | | | | (B) | | (U) | | |
| | 47 (21 | 4.0 | 0.1 | 4.0 | 4.29 | 121 | 0.236 | 114 | 0.260 | Excellent |
| | days) | | | | | (U) | | (U) | | |
| | 51 (2 | 4.0 | 0.1 | 4.0 | 4.89 | 115 | 0.254 | 114 | 0.271 | Excellent |
| | days) | | | | | (U) | | (U) | | |
| | 52 (2 | 4.0 | 0.1 | 4.0 | 4.84 | 323 | 0.885 | 322 | 0.454 | Not stable |
| | days) | | | | | (M) | | (M) | | |
| | 48 (21 | 4.0 | 0.1 | 4.0 | 4.34 | 110 | 0.248 | 108 | 0.310 | Excellent |
| | days) | | | | | (U) | | (U) | | |
| | 32 (60 | 4.0 | 0.1 | 5.2 | 6.44 | 61 | 0.245 | 67 | 0.251 | Excellent |
| | days) | | | | | (U) | | (U) | | |

The serum stability of DNPs assembled from IAJD33+IAJD32 (2%) was determined. Sample preparation: injected 25 μL ethanol stock solution of IAJD33+IAJD32 (2%) (80 mg/mL of IAJDs) into the mixture of 462.5 μL acetate buffer (10 mM, pH 4.0) and 12.5 μL mRNA (4.0 mg/mL in water), followed by vortex for 5 seconds.

Final composition of DNP solution: IAJD33+IAJD32 (2%) (4.0 mg/mL)+mRNA (0.1 mg/mL) in 500 μL acetate buffer. The dimension of assemblies was checked by DLS.

The 500 μL DNP solution was dialyzed against 10 mM tris buffer (10 mM, pH 7.4) for 120 minutes in 3,500-14,000 molecular weight cut-off dialysis tube (Spectrum Medical Instruments Inc. Spectra/Por molecular porous membrane tubing Flat Width: 45 mm; Diameter: 29 mm & Vol/length: 6.4 mL/cm).

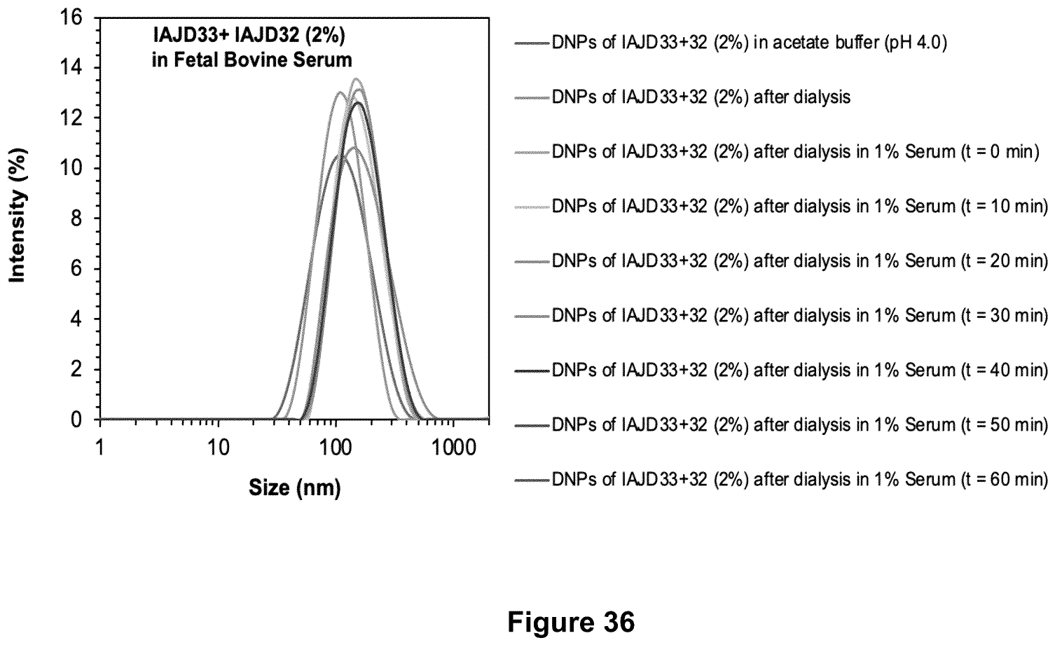

DNPs in 1% Serum: Serum (5.5 μL)+[IAJDs (4.0 mg/mL)+mRNA (0.1 mg/mL) in 500 μL solution after dialysis in tris buffer]. The dimensions of the resulting DNPs are presented in Table 10 and FIG. 36.

TABLE 10

| Samples | Size (nm) | PDI |
|---|---|---|
| DNPs of IAJD33 + 32 (2%) | 92 | 0.352 |
| in acetate buffer (pH 4.0) | | |
| DNPs of IAJD33 + 32 (2%) after dialysis | 105 | 0.170 |
| DNPs of IAJD33 + 32 (2%) | 146 | 0.150 |
| after dialysis in 1% Serum (t = 0 min) | | |
| DNPs of IAJD33 + 32 (2%) | 147 | 0.183 |
| after dialysis in 1% Serum (t = 10 min) | | |
| DNPs of IAJD33 + 32 (2%) | 145 | 0.180 |
| after dialysis in 1% Serum (t = 20 min) | | |
| DNPs of IAJD33 + 32 (2%) | 148 | 0.171 |
| after dialysis in 1% Serum (t = 30 min) | | |
| DNPs of IAJD33 + 32 (2%) | 144 | 0.161 |
| after dialysis in 1% Serum (t = 40 min) | | |
| DNPs of IAJD33 + 32 (2%) | 145 | 0.154 |
| after dialysis in 1% Serum (t = 50 min) | | |
| DNPs of IAJD33 + 32 (2%) | 143 | 0.141 |
| after dialysis in 1% Serum (t = 60 min) | | |

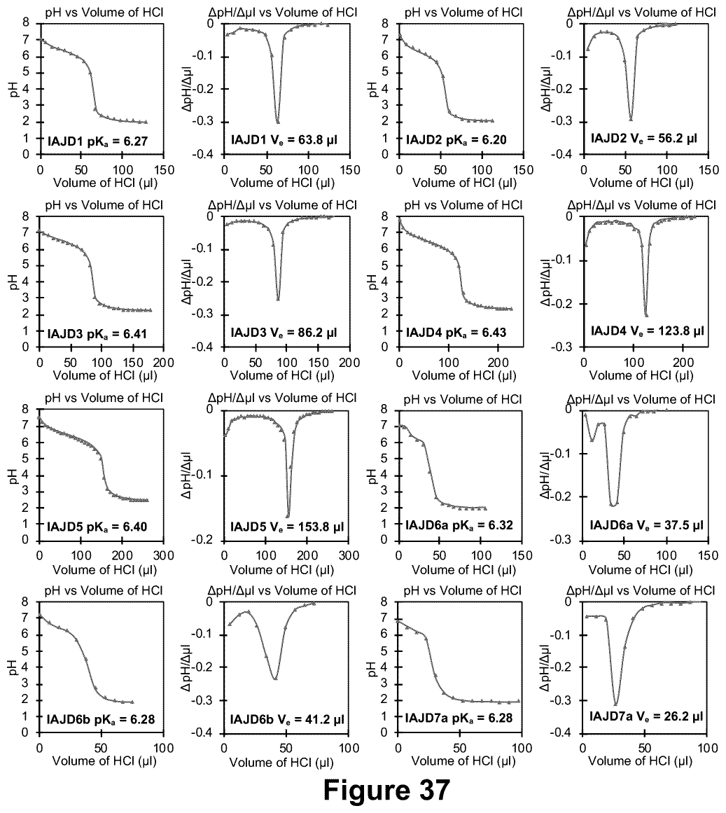

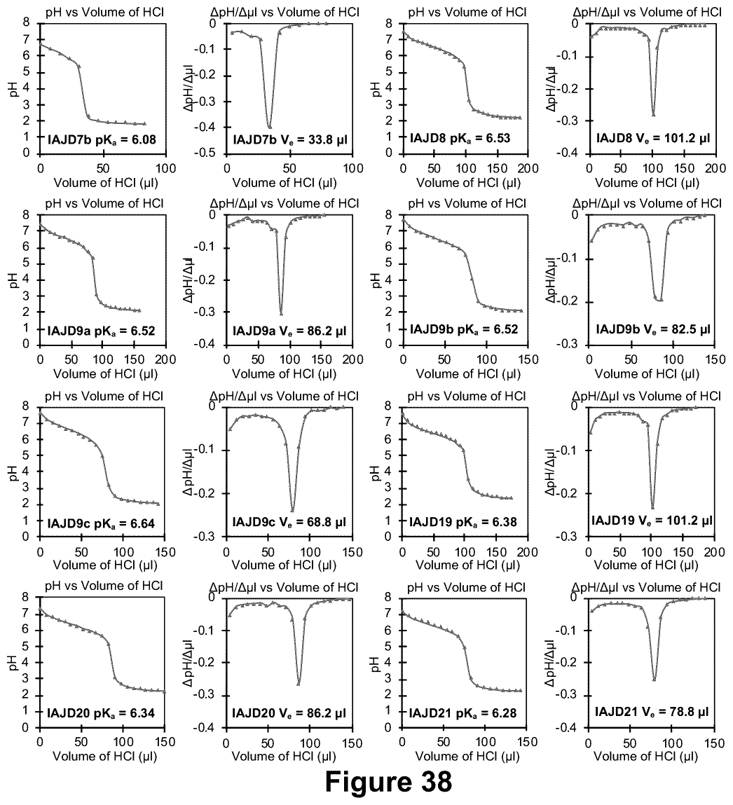

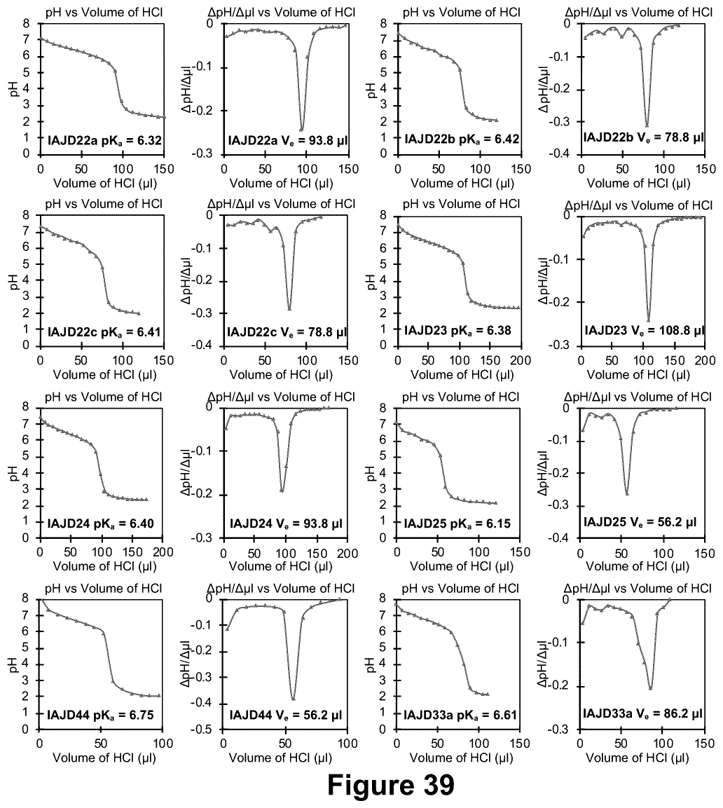

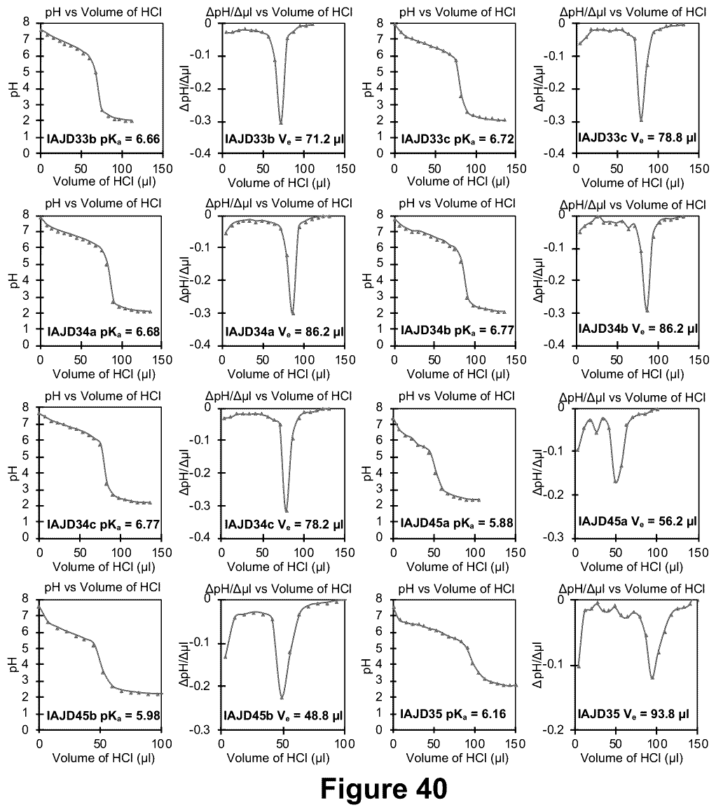

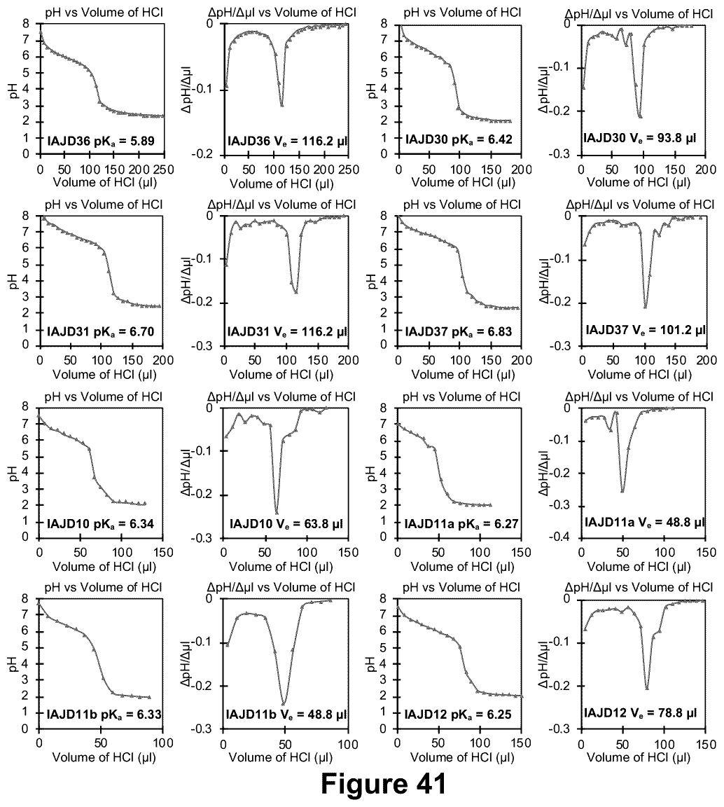

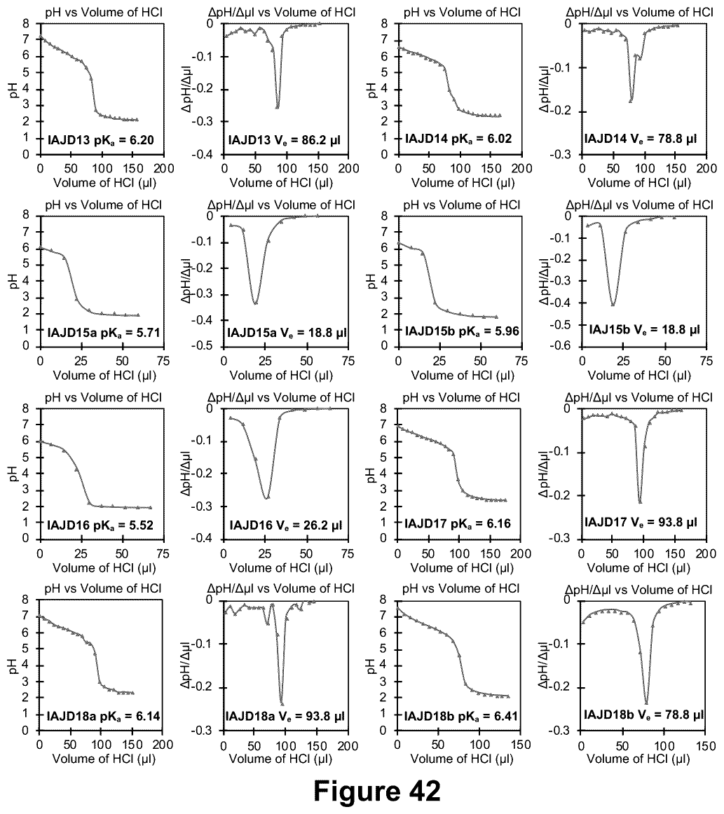

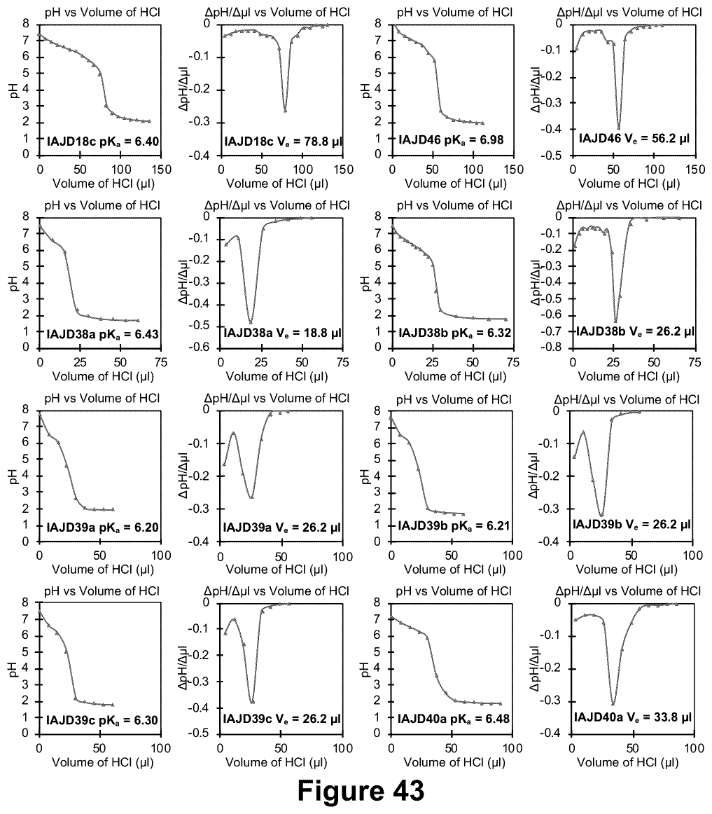

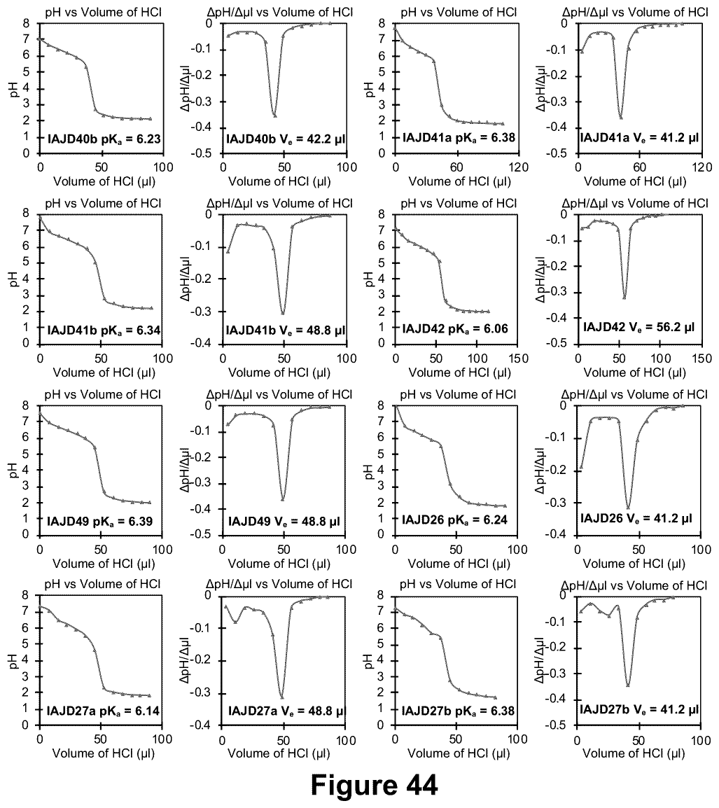

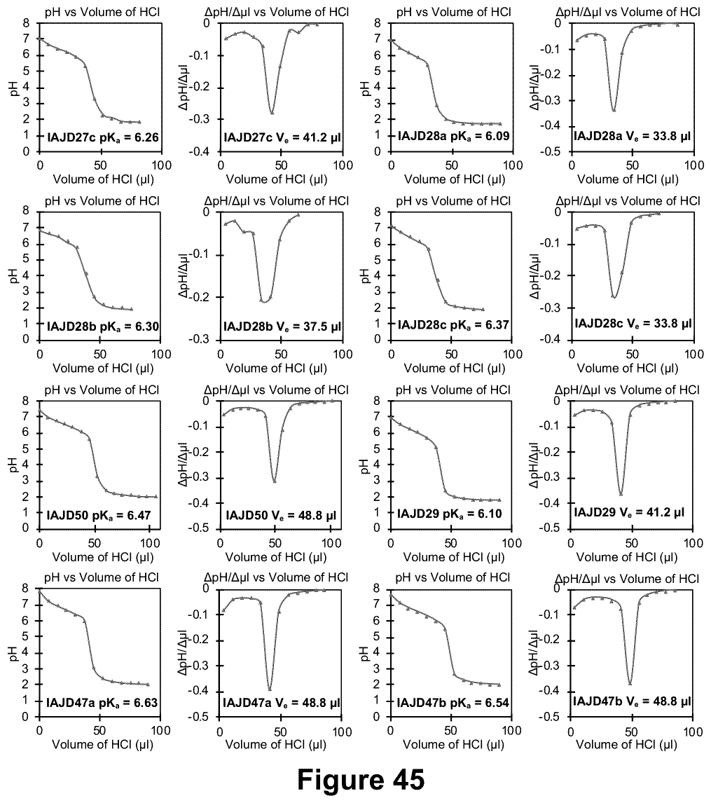

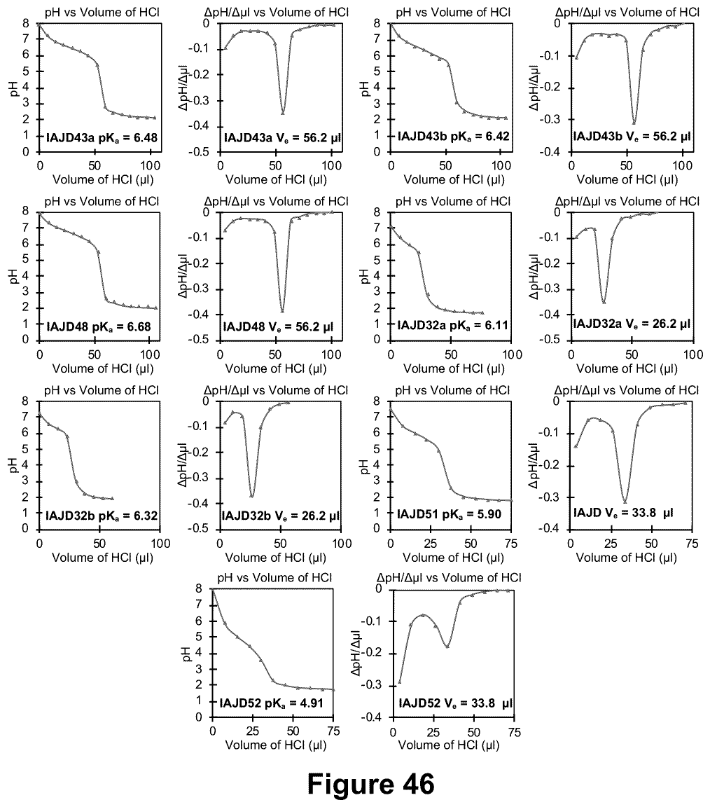

The pKₐ values of the individual IAJD molecules was assayed. The results for each library are presented in Table 11. Titration curves for each compound are presented in FIG. 37 through FIG. 46.

TABLE 11
pKₐ of Individual IAJDs Molecules

| | | | | | |
|---|---|---|---|---|---|
| IAJD No. | pKₐ 1 | pKₐ 2 | pKₐ 3 | Avg pKₐ | Std Div |

Library 1

| | | | | | |
|---|---|---|---|---|---|
| IAJD 1 | 6.27 | | | 6.27 | |
| IAJD 2 | 6.20 | | | 6.20 | |
| IAJD 3 | 6.41 | | | 6.41 | |
| IAJD 4 | 6.43 | | | 6.43 | |
| IAJD 5 | 6.40 | | | 6.40 | |
| IAJD 6 | 6.32 | 6.28 | | 6.30 | 0.03 |
| IAJD 7 | 6.28 | 6.08 | | 6.18 | 0.14 |
| IAJD 8 | 6.53 | | | 6.53 | |
| IAJD 9 | 6.52 | 6.52 | 6.64 | 6.56 | 0.07 |

Library 2

| | | | | | |
|---|---|---|---|---|---|
| IAJD 19 | 6.38 | | | 6.38 | |
| IAJD 20 | 6.34 | | | 6.34 | |
| IAJD 21 | 6.28 | | | 6.28 | |
| IAJD 22 | 6.32 | 6.42 | 6.41 | 6.38 | 0.06 |
| IAJD 23 | 6.38 | | | 6.38 | |
| IAJD 24 | 6.40 | | | 6.40 | |
| IAJD 25 | 6.15 | | | 6.15 | |

Library 3

| | | | | | |
|---|---|---|---|---|---|
| IAJD 44 | 6.75 | | | 6.75 | |
| IAJD 33 | 6.61 | 6.66 | 6.72 | 6.66 | 0.06 |
| IAJD 34 | 6.68 | 6.77 | 6.77 | 6.74 | 0.05 |
| IAJD 45 | 5.88 | 5.98 | | 5.93 | 0.07 |
| IAJD 35 | 6.16 | | | 6.16 | |
| IAJD 36 | 5.89 | | | 5.89 | |

Library 4

| | | |
|---|---|---|
| IAJD 30 | 6.42 | 6.42 |
| IAJD 31 | 6.70 | 6.70 |
| IAJD 37 | 6.83 | 6.83 |

Library 5

| | | | | | |
|---|---|---|---|---|---|
| IAJD 10 | 6.34 | | | 6.34 | |
| IAJD 11 | 6.27 | 6.33 | | 6.30 | 0.04 |
| IAJD 12 | 6.25 | | | 6.25 | |
| IAJD 13 | 6.20 | | | 6.20 | |
| IAJD 14 | 6.02 | | | 6.02 | |
| IAJD 15 | 5.71 | 5.96 | | 5.84 | 0.18 |
| IAJD 16 | 5.52 | | | 5.52 | |
| IAJD 17 | 6.16 | | | 6.16 | |
| IAJD 18 | 6.14 | 6.41 | 6.40 | 6.32 | 0.15 |
| IAJD 46 | 6.98 | | | 6.98 | |

Library 6

| | | | | | |
|---|---|---|---|---|---|
| IAJD 38 | 6.43 | 6.32 | | 6.38 | 0.07 |
| IAJD 39 | 6.20 | 6.21 | 6.30 | 6.24 | 0.06 |
| IAJD 40 | 6.48 | 6.23 | | 6.36 | 0.18 |
| IAJD 41 | 6.38 | 6.34 | | 6.36 | 0.03 |
| IAJD 42 | 6.06 | | | 6.06 | |
| IAJD 49 | 6.39 | | | 6.39 | |
| IAJD 26 | 6.24 | | | 6.24 | |
| IAJD 27 | 6.14 | 6.38 | 6.26 | 6.26 | 0.12 |
| IAJD 28 | 6.09 | 6.30 | 6.37 | 6.25 | 0.15 |
| IAJD 50 | 6.47 | | | 6.47 | |
| IAJD 29 | 6.10 | | | 6.10 | |
| IAJD 47 | 6.63 | 6.54 | | 6.59 | 0.06 |
| IAJD 51 | 5.90 | | | 5.90 | |
| IAJD 52 | 4.91 | | | 4.91 | |
| IAJD 43 | 6.48 | 6.42 | | 6.45 | 0.04 |
| IAJD 48 | 6.68 | | | 6.68 | |
| IAJD 32 | 6.11 | 6.32 | | 6.22 | 0.15 |

### Example 2: Targeted Delivery of mRNA with One-Component IAJDs

One aspect of the invention relates in part to a one-component multifunctional sequence-defined IAJD delivery system that co-assembles with mRNA by simple injection into DNPs (FIG. 1). IAJDs described in the preceding examples require a 14-step synthesis for their preparation. Earlier IAJDs use a combination of a hydrophobic dendron conjugated to a hydrophilic dendron based on methoxytriethylene glycol fragments (Z) enclosing an ionizable amine incorporated in a sequence-defined arrangement. In order to reduce the number of synthetic steps employed in the construction of IAJDs, the methoxytriethylene glycol fragments (Z) attached to the branching point of the hydrophilic fragment of the JD were eliminated since these fragments required multiple protection-deprotection steps. In the IAJDs described in the following examples only its ionizable amine is maintained, which when protonated, may provide the dual hydrophilic and binding component of the IAJD to mRNA (FIG. 47 through FIG. 49). This transformation reduced the number of reaction steps significantly. The synthesis is shown in Scheme 1.

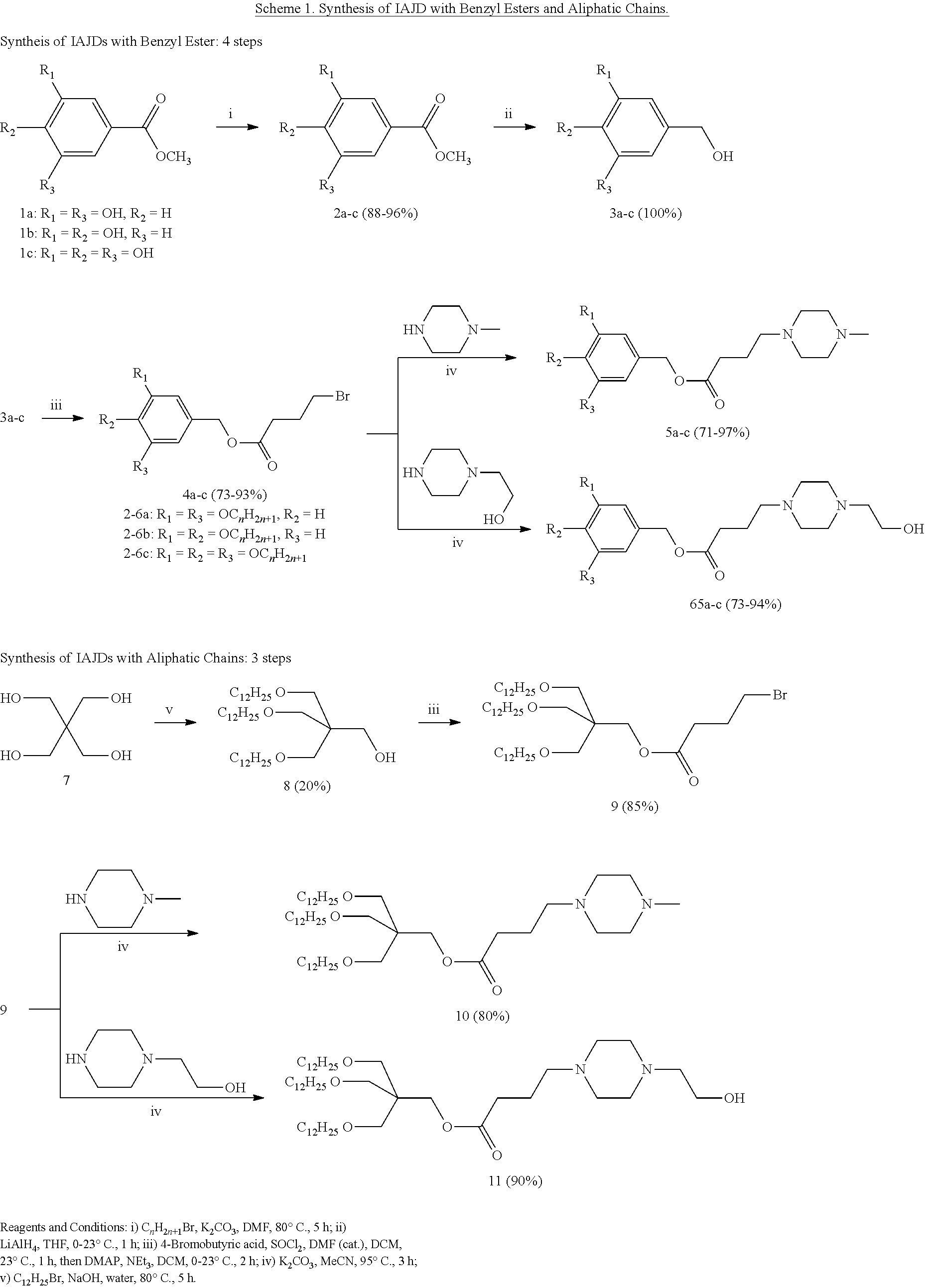

The materials and methods employed in these experiments are now described.

### Materials

3,5-Dihydroxybenzoic acid (Acros, 97%), 3,4-dihydroxybenzoic acid (Acros, 97%), 3,4,5-trihydroxybenzoic acid (gallic acid, Chem Impex, anhydrous, ACS grade), 1-bromooctane (Aldrich, 99%), 1-bromononane (Lancaster, 99%), 1-bromodecane (Acros, 98%), 1-bromoundecane (Aldrich, 99%), 1-bromododecane (Alfa Aesar, 99%), 1-bromotetradecane (Acros, 98%), 1-bromohexadecane (TCI, 96%), 1-bromooctadecane (Acros, 96%), (rac)-3-(bromomethyl)heptane (2-ethylhexyl bromide, Aldrich, 95%), (rac)-1-bromo-3,7-dimethyloctane (TCI, 93+%), LiAlH₄ (TCI, 95%), palladium on activated carbon catalyst (Spectrum, 10 wt % loading), pentaerythritol (Aldrich, 98%), 4-(dimethylamino)butyric acid hydrochloride (Alfa Aesar, 98%), 3-bromopropanoic acid (Aldrich, 97%), 4-bromobutyric acid (Acros, 98%), 5-bromovaleric acid (Aldrich, 97%), 1-methylpiperazine (Alfa Aesar, 98%). 1-(2-hydroxyethyl)piperazine (Acros, 99%), thionyl chloride (Alfa Aesar, 99+%), triethylamine (TCI, 99%), other reagents and solvents for chemical synthesis were obtained from commercial sources and were used as received. 4-(Dimethylamino)pyridinium 4-toluenesulfonate (DPTS) was prepared according to a literature procedure.¹ CH₂Cl₂ (DCM) was dried over CaH2 and freshly distilled before use. Acetate buffer (10 mM) was prepared with the following composition: sodium acetate (0.0023 M) and acetic acid (0.0077 M) in ultra-pure water. Final pH was adjusted with 0.1 M NaOH or 0.1 M HCl solution.

Nucleoside-modified mRNA encoding firefly luciferase (Luc-mRNA) was produced as previously described. Human embryonic kidney (HEK) 293T cells (American Type Culture Collection) were cultured in Dulbecco's modified Eagle's medium (DMEM) supplemented with 10% inactivated fetal bovine serum (FBS) (Gemini Bio-Products), 2 mM L-glutamine and 100 U/mL penicillin/streptomycin (Life Technologies). OptiMEM (Gibco), DPBS (Corning), UltraPure DNase/RNase-Free Distilled Water (Invitrogen), Trypan Blue (Sigma-Aldrich), Trypsin-EDTA (0.25%), (Gibco), Cell Culture Lysis 5× Reagent (Promega), Luciferase Assay System (Promega) and D-luciferin sodium salt (Regis Technologies) were used as received.

### Methods and Techniques

The purity and structural identity of intermediate compounds and final products and was determined by a combination of techniques including thin-layer chromatography (TLC), ¹H and ¹³C NMR, high-pressure liquid chromatography (HPLC), and matrix assisted laser desorption ionization-time of flight (MALDI-TOF) mass spectrometry as described above.

Evolution of the reaction was monitored by TLC using silica gel 60 F254 precoated plates (E. Merck) and individual compounds were visualized by UV light with a wavelength of 254 nm or by staining of the TLC plate with iodine vapor. Purification by flash column chromatography was performed using flash silica gel from Silicycle (60 Å, 40-63 μm) with the indicated eluent.

Dynamic Light Scattering (DLS) for the sizes and polydispersities of DNPs was performed on a Malvern Instruments particle sizer (Zetasizer Nano S, Malvern Instruments, UK) equipped with 4 mW He—Ne laser 633 nm and avalanche photodiode positioned at 1750 to the beam and temperature-controlled cuvette holder. Instrument parameters were determined automatically along with measurement times. Sample solution (0.4 mL) was placed in a semi-micro cuvette (1.6 mL, polystyrene, 10×10×45 mm, manufactured by Greiner Bio-One) and the measurements were performed at 23° C.

pKₐ measurements of individual IAJD Molecules was performed as follows. IAJD Molecules were first dissolved in ethanol (Sat. with NaCl) at a concentration of 1.5 mg/mL and the solution volume was 3 mL. Then 0.1 M HCl aqueous solution was added to the above ethanol solution in increments of 7.5 μL, with the resulting pH measured using an Thermo Scientific Orion Star A121 meter with Thermo Scientific Orion 8220BNWP pH probe. pKₐ was calculated using the half equivalence point titration.

Formulation of DNPs obtained by co-assembly of IAJDs and Luc-mRNA was performed as follows. IAJDs were dissolved in ethanol with an initial concentration of 80 mg/mL. Nucleoside-modified mRNA encoding firefly luciferase (Luc-mRNA) was dissolved in UltraPure DNase/RNase-free distilled water with an initial concentration of 4.0 mg/mL. 12.5 μL of Luc-mRNA solution was placed into a clean RNAs free eppendorf (1.5 mL) and 463 μL acetate buffer (10 mM, pH 4.0) was added. Then, 25 μL of IAJD in the ethanol stock solution was rapidly injected into the Luc-mRNA solution in acetate buffer followed by vortex for 5 seconds.

Luminescence characterization for in vitro transfection experiments was performed by determining the luminescence intensity for in vitro Luc-mRNA transfection experiments using a MiniLumat LB 9506 luminometer (Berthold/EG&G; Wallac).

Luminescence characterization for in vivo transfection experiments employed bioluminescence imaging with an IVIS Spectrum imaging system (PerkinElmer, Waltham, MA). Mice were anesthetized with 3% of isoflurane (Piramal Healthcare Limited) and intraperitoneally (i.p.) administered with D-luciferin (Regis Technologies) at a dose of 150 mg/kg of body weight. Ten minutes post administration of D-luciferin, mice were placed on the imaging platform while being maintained on isoflurane via a nose cone and imaged using an exposure time of 60 seconds. Bioluminescence values were quantified by measuring photon flux (photons/second, p/s) in the region of interest (ROI) on mice where bioluminescence signal emanated using the Living Image Software (PerkinElmer). To quantify luminescent flux, an oval ROI was placed over each organ of interest and analyzed.

For in vitro Luc-mRNA transfection screening experiments, human embryonic kidney 293 cells (HEK 293) were seeded into 96-well plates (20,000 cells/well/200 μL) and cultured for 18-20 hours at 37° C., 5% CO₂ in complete Dulbecco's modified Eagle's medium (DMEM) supplemented with 2 mM L-glutamine (Life Technologies), 100 U/mL penicillin/streptomycin (Invitrogen) and 10% fetal bovine serum (FBS, Gemini Bio-Products). Cells were transfected with DNPs with encapsulated naked nucleoside-modified mRNA encoding firefly luciferase (Luc-mRNA) at constant concentration of Luc-mRNA, 125 ng per well. Luc-mRNA complexed with TransIT (TransIT®-mRNA Transfection Kit, Mirus Bio) or encapsulated in FDA approved MC3-based LNPs were used as positive controls for cell transfection, at the concentration of Luc-mRNA of 125 ng per well, the same as for tested DNPs. After transfection cells were further cultured at 37° C., 5% CO2 for 18-20 hours, then medium was aspirated and cells were lysed with luciferase cell culture lysis reagent (30 μL/well) (Promega, Madison, WI). For the determination of the luciferase enzymatic activity as luminescence, 2.5 μL of the lysed cells was mixed with 10 μL of firefly luciferase assay substrate ((Luciferase assay system, Promega) and luminescence was analyzed by MiniLumat LB9506 luminometer. Transfections were performed in triplicate.

In vivo mRNA Delivery in Mice with DNPs employed female or male BALB/c mice (6-8 weeks old, from Charles River Laboratories) anesthetized with isoflurane (Piramal Healthcare Limited) and injected via retro-orbital sinus with 100 μL of buffer solution containing DNPs with encapsulated 10 pg of Luc-mRNA. At 4-7 hours post injection, mice were i.p. injected with D-Luciferin (150 mg/kg of body weight, Regis Technologies) and imaged on a PerkinElmer IVIS Spectrum CT system (PerkinElmer, Waltham, MA). Tissue luminescence signal was measured on the IVIS imaging system using an exposure time of 60 seconds using medium binning (binning=8) to ensure that the signal obtained was within operative detection range. For IVIS imaging of the organs, mice were sacrificed and heart, lungs, liver, and spleen were immediately collected. Bioluminescence imaging was performed as described above. Image analysis was conducted with the Living Image software (PerkinElmer). Bioluminescence values were quantified by measuring photon flux (photons/s) in the region of interest using Living Image software.

Synthesis of IAJDs with ester bonds in hydrophobic alkyl chains was performed as shown in Scheme 2.

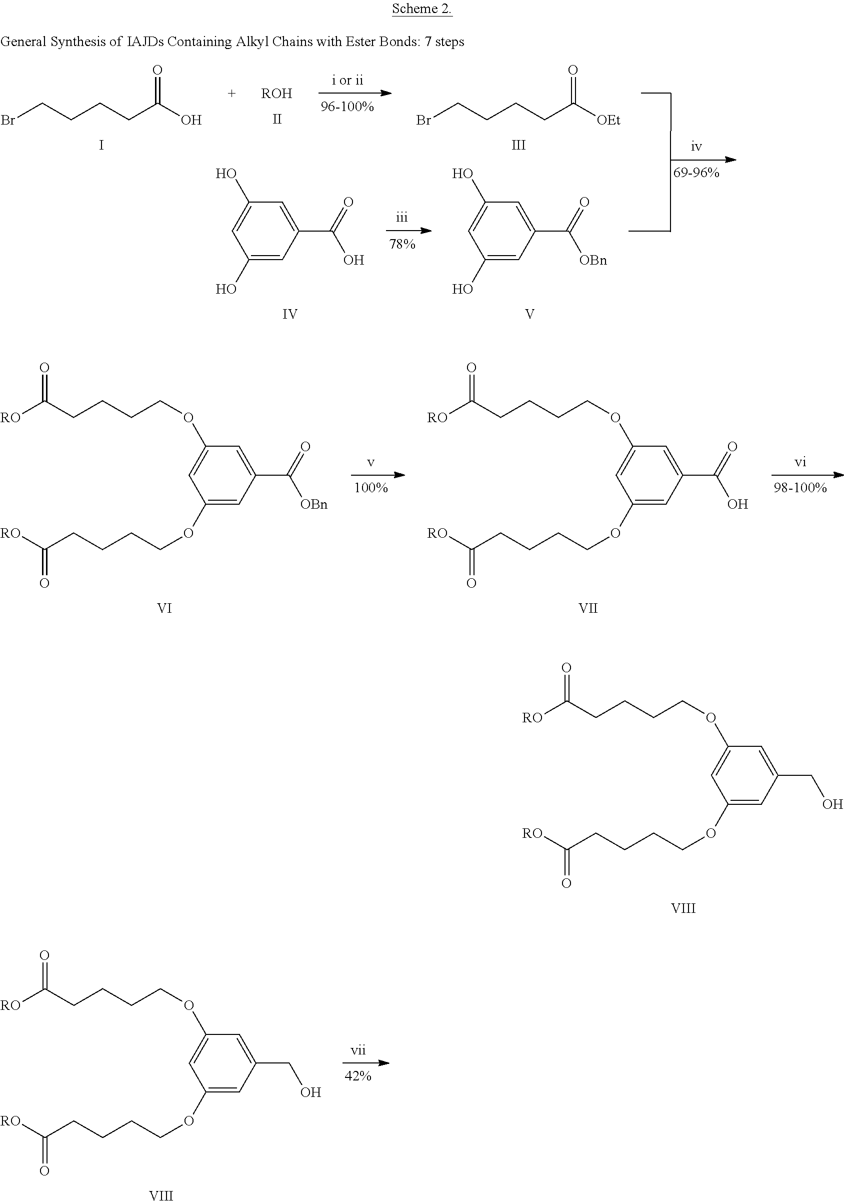

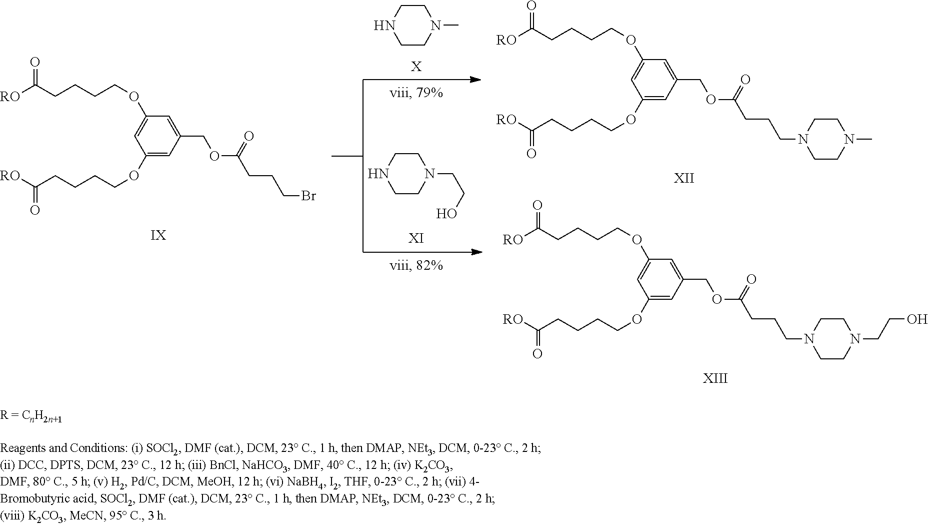

Synthesis of Hydrophobic Building Blocks of IAJDs was performed as shown in Scheme 3.

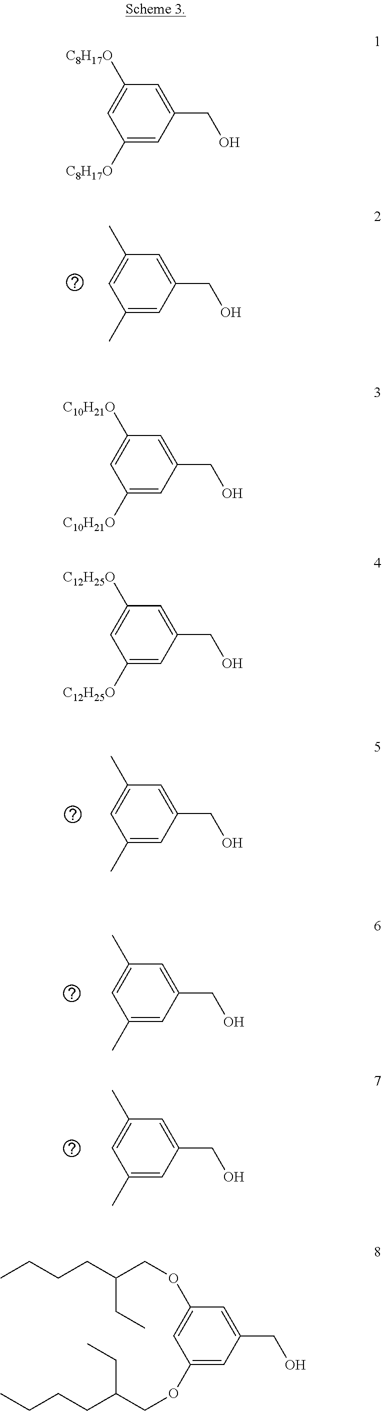

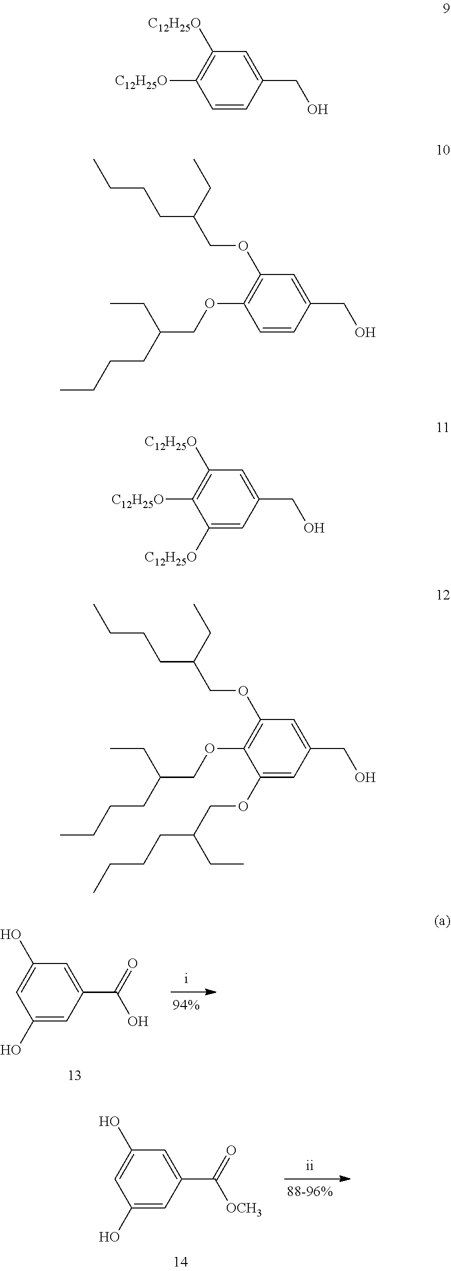

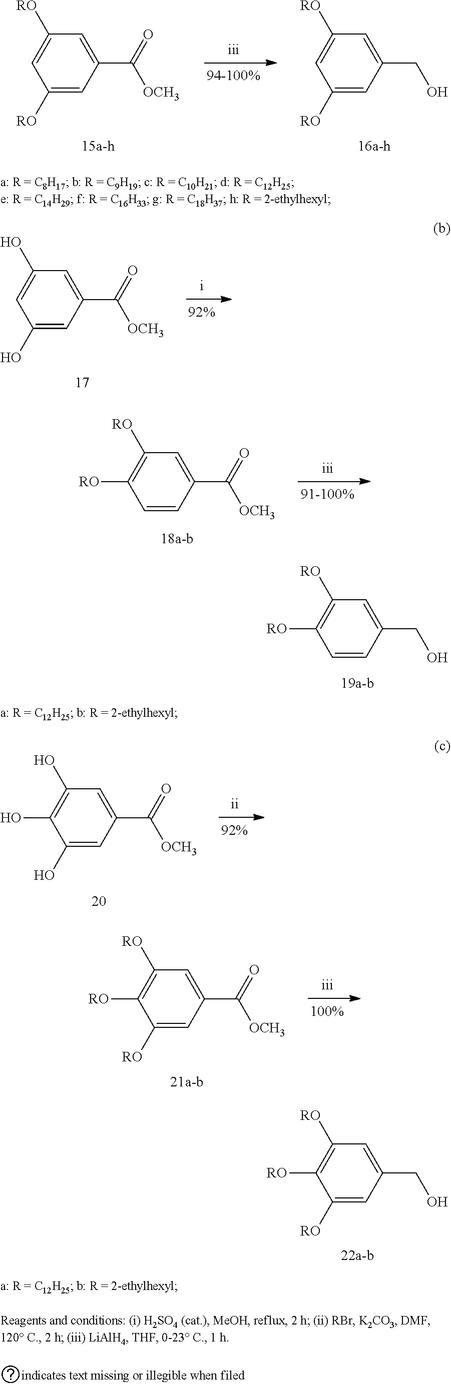

General Synthetic Procedure for Compounds 15a-h, 18a-b, and 21a-b: Compounds 15a-h, 18a-b, and 21a-b were synthesized according to a procedure elaborated and optimized. Generally, compound 14/17/20 (1 equiv) and K₂CO₃ (3 equiv for compound 14/17, 4.5 equiv for compound 20) were stirred in dry DMF. RBr (2.2 equiv for compound 14/17, 3.3 equiv for compound 20) was added, and the mixture was stirred at 120° C. under N₂ atmosphere for 2 h. The reaction mixture was cooled to 23° C. For compounds 15a-g, 18a and 21a, the reaction mixture was poured into ice/water and the white precipitates were filtered and collected. Then the precipitates were purified by recrystallization from acetone to give the title compound as a white solid. For compounds 15h, 18b and 21b, water was added to the reaction mixture, which was then extracted by DCM for three times. The organic phase was collected and dried over anhydrous MgSO₄ and filtered. The filtrate was concentrated and further purified by column chromatography on silica gel with a mobile phase of hexane/EtOAc=20/1 (v/v) to afford the title compound as a light-yellow oil.

General Synthetic Procedure for Compounds 16a-h (1-8), 19a-b (9,10), and 22a-b (11,12): Compounds 16a-h, 19a-b, and 22a-b were synthesized according to a procedure previously reported by us. Generally, compounds 15a-h, 18a-b, and 21a-b (1 equiv) were dissolved in dry THF, which was added dropwise to a slurry of LiAlH₄ (1.2 equiv) in dry THF at 0° C. under N₂ atmosphere. The resulted mixture was stirred at 23° C. for 1 h. The reaction was quenched by the successive addition of water, 15% NaOH aqueous solution and water. Then the mixture was filtered and dried over anhydrous MgSO₄. Filtration and evaporation of the solvent yielded the title compound as a white solid or light-yellow oil. The synthesis and characterizations of compounds 16d (4), 19a (9) and 22a (11) were reported in the literature.

### (3,5-Bis(octyloxy)phenyl)methanol (1)

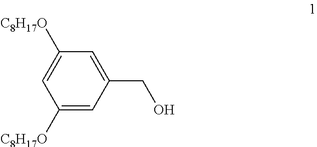

From compound 15a (4.50 g, 11.46 mmol) and LiAlH₄ (0.52 g, 13.75 mmol), compound 1 was obtained as a colorless oil (4.10 g, 98%).

### (3,5-Bis(nonyloxy)phenyl)methanol (2)

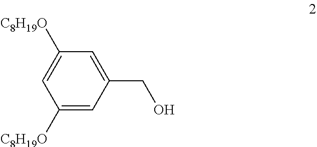

From compound 15b (1.16 g, 2.75 mmol) and LiAlH₄ (0.13 g, 3.30 mmol), compound 2 was obtained as a colorless oil (1.08 g, 100%).

### (3,5-bis(decyloxy)phenyl)methanol (3)

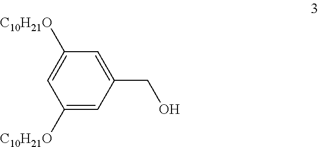

From compound 15c (3.40 g, 8.08 mmol) and LiAlH₄ (0.37 g, 9.70 mmol), compound 3 was obtained as a white solid (3.21 g, 94%).

### (3,5-Bis(tetradecyloxy)phenyl)methanol (5)

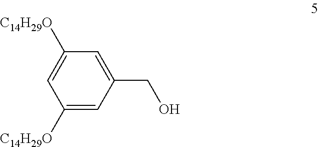

From compound 15e (0.50 g, 0.89 mmol) and LiAlH₄ (51 mg, 1.34 mmol), compound 5 was obtained as a white solid (0.48 g, 100%).

### (3,5-Bis(hexadecyloxy)phenyl)methanol (6)

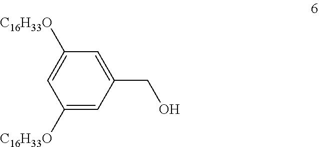

From compound 15f (0.50 g, 0.81 mmol) and LiAlH₄ (46 mg, 1.22 mmol), compound 6 was obtained as a white solid (0.48 g, 100%).

### (3,5-Bis(octadecyloxy)phenyl)methanol (7)

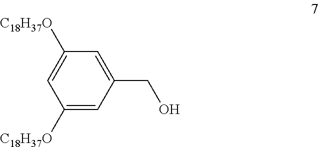

From compound 15g (0.60 g, 0.89 mmol) and LiAlH₄ (68 mg, 1.78 mmol), compound 7 was obtained as a white solid (0.55 g, 96%).

### (3,5-Bis((2-ethylhexyl)oxy)phenyl)methanol (8)

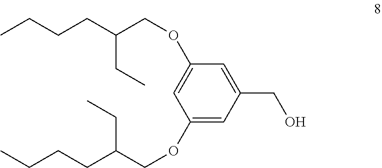 [SMILES: CCCCC(CC)COc1cc(CO)cc(OCC(CC)CCCC)c1]

From compound 15h (4.10 g, 10.44 mmol) and LiAlH₄ (0.48 g, 12.53 mmol), compound 8 was obtained as a light-yellow oil (3.64 g, 96%).

### (3,4-Bis((2-ethylhexyl)oxy)phenyl)methanol (10)

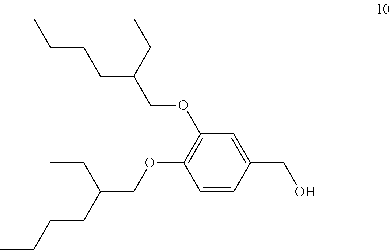 [SMILES: CCCCC(CC)COc1ccc(CO)cc1OCC(CC)CCCC]

From compound 18b (2.77 g, 7.10 mmol) and LiAlH₄ (0.32 g, 8.50 mmol), compound 10 was obtained as a colorless oil (2.36 g, 91%).

### (3,4,5-Tris((2-ethylhexyl)oxy)phenyl)methanol (12)

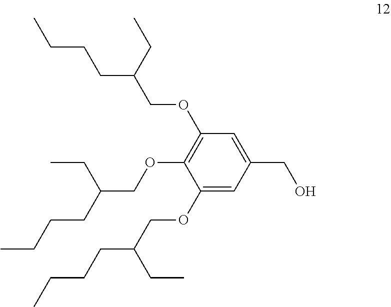 [SMILES: CCCCC(CC)COc1cc(CO)cc(OCC(CC)CCCC)c1OCC(CC)CCCC]

From compound 21b (0.60 g, 1.15 mmol) and LiAlH₄ (0.12 g, 3.16 mmol), compound 12 was obtained as a colorless oil (0.57 g, 100%).

Synthesis of IAJDs 64-66 and 70 is shown in Scheme 4.

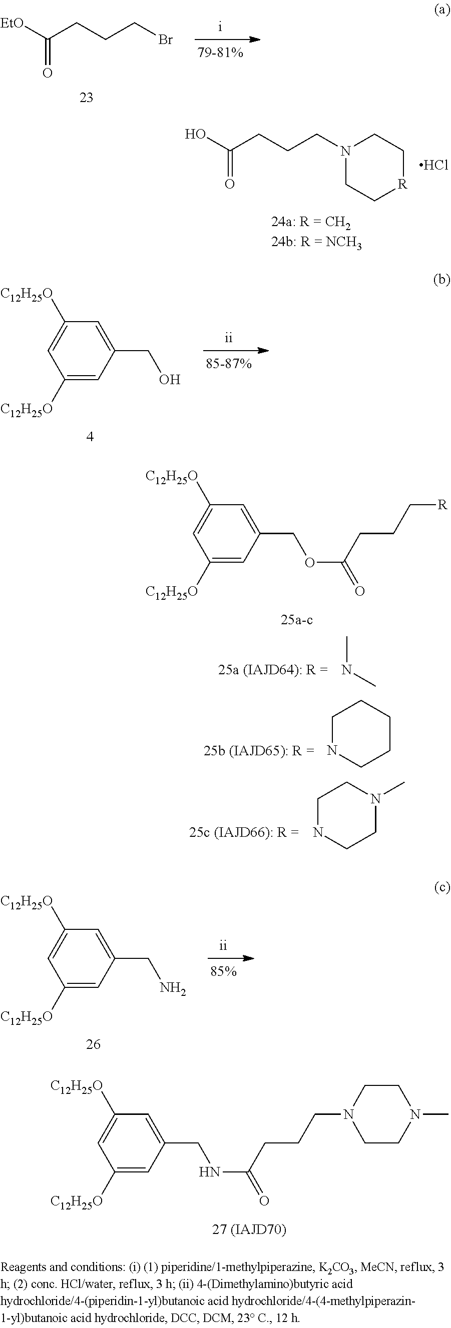

The synthesis and characterizations of compounds 24a and 24b are available in the literature.

### 3,5-Bis(dodecyloxy)benzyl 4-(dimethylamino)butanoate (25a, IAJD64)

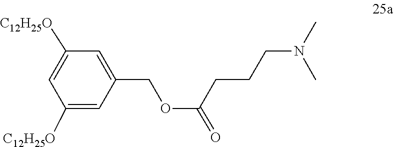

Compound 4 (0.50 g, 1.05 mmol, 1 equiv) and 4-(dimethylamino)butyric acid hydrochloride (0.18 g, 1.05 mmol, 1 equiv) were dissolved in 6 mL dry DCM. N,N′-Dicyclohexylcarbodiimide (DCC, 0.43 g, 2.10 mmol, 2 equiv) was added in one portion into the above mixture. The reaction was allowed to stir at 23° C. for 12 h. Afterwards, urea was removed by filtration, and washed with DCM carefully. The filtrate was concentrated and purified by column chromatography (SiO₂) with DCM/MeOH=40/1, 20/1 and 15/1 as the eluent. Then the product was dissolved in DCM (30 mL), which was washed by NaHCO₃solution (2%, 30 mL). The aqueous phase was extracted by DCM (30 mL) for another two times. The organic phase was combined and dried over anhydrous MgSO₄. Filtration and evaporation of the solvent yielded the title compound as a colorless oil (0.54 g, 87%).

### 3,5-Bis(dodecyloxy)benzyl 4-(piperidin-1-yl)butanoate (25b, IAJD65)

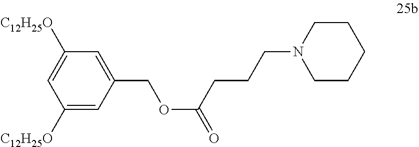

Compound 25b was synthesized from compound 4 (0.50 g, 1.05 mmol, 1 equiv), 4-(piperidin-1-yl)butanoic acid hydrochloride (24a, 0.22 g, 1.05 mmol, 1 equiv) and DCC (0.43 g, 2.10 mmol, 2 equiv) following a procedure similar to that used for the synthesis of compound 25a. The title compound was obtained as a colorless oil (0.57 g, 86%).

### 3,5-Bis(dodecyloxy)benzyl 4-(4-methylpiperazin-1-yl)butanoate (25c, IAJD66)

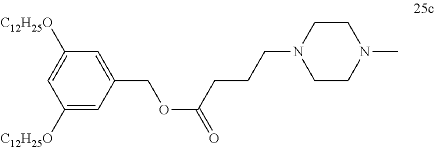

Compound 25c was synthesized from compound 4 (0.20 g, 0.42 mmol, 1 equiv), 4-(4-methylpiperazin-1-yl)butanoic acid hydrochloride (24b, 94 mg, 0.42 mmol, 1 equiv) and DCC (0.17 g, 0.84 mmol, 2 equiv) following a procedure similar to that used for the synthesis of compound 25a. The title compound was obtained as a colorless oil (0.23 g, 85%).

### N-(3,5-bis(dodecyloxy)benzyl)-4-(4-methylpiperazin-1-yl)butanamide (27, IAJD70)

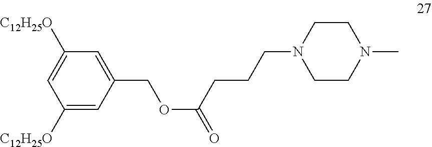

The synthesis and characterizations of (3,5-bis(dodecyloxy)phenyl)methanamine (26) were reported in the literature.⁷ Compound 26 (0.20 g, 0.42 mmol, 1 equiv) and 4-(4-methylpiperazin-1-yl)butanoic acid hydrochloride (24b, 94 mg, 0.42 mmol, 1 equiv) were dissolved in 6 mL dry DCM. NEt₃ (3 drops) was added. DCC (0.17 g, 0.84 mmol, 2 equiv) was added in one portion into the above mixture. The reaction was allowed to stir at 23° C. for 12 h. Afterwards, urea was removed by filtration, and washed with DCM carefully. The filtrate was concentrated and purified by column chromatography (SiO₂) with DCM/MeOH=30/1, 10/1, 8/1 and 8/1 with 0.1% TEA as the eluent. Then the product was dissolved in DCM (30 mL), which was washed by NaHCO₃solution (2%, 30 mL). The aqueous phase was extracted by DCM (30 mL) for another two times. The organic phase was combined and dried over anhydrous MgSO₄. Filtration and evaporation of the solvent yielded the title compound as a white solid (0.23 g, 85%).

Synthesis of IAJDs 71 and 74-79 is shown in Scheme 5.

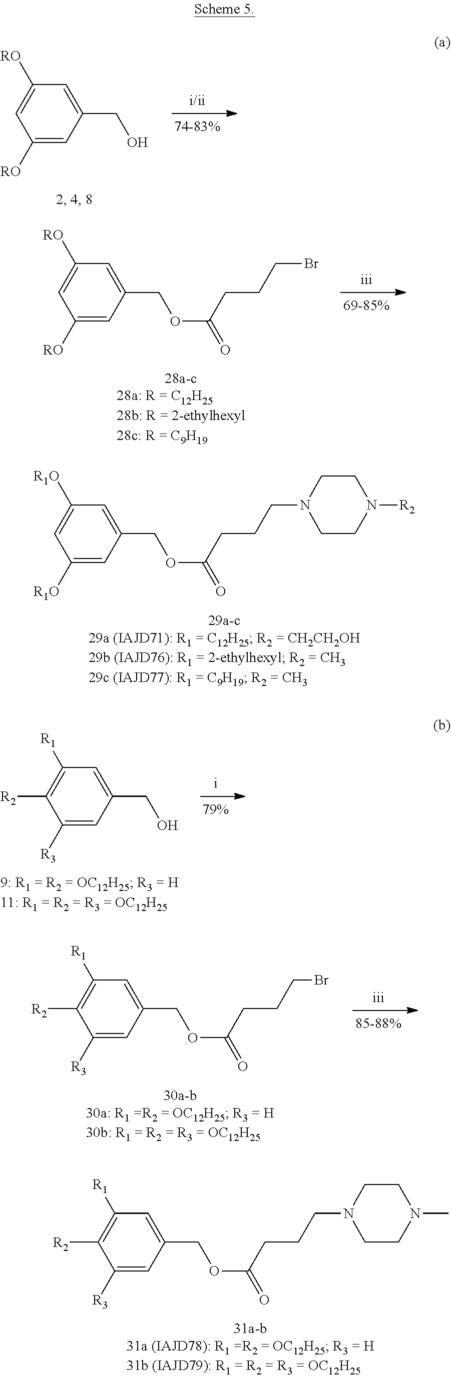

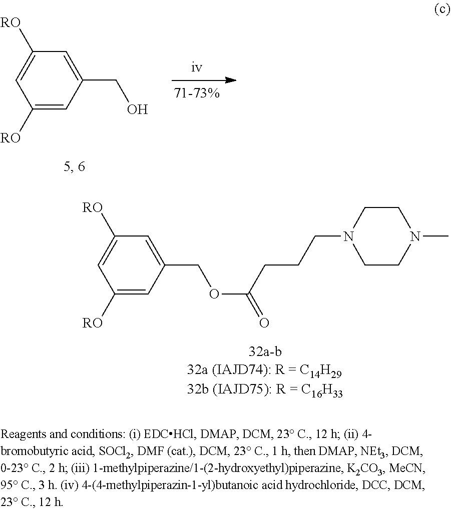

3,5-Bis(dodecyloxy)benzyl 4-bromobutanoate (28a).

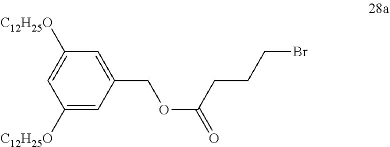

Compound 4 (0.60 g, 1.26 mmol, 1 equiv), 4-bromobutyric acid (0.21 g, 1.26 mmol, 1 equiv), EDC·HCl (0.24 g, 1.26 mmol, 1 equiv) and DMAP (46 mg, 0.38 mmol, 0.3 equiv) were dissolved in dry DCM (6 mL). The reaction mixture was stirred at 23° C. for 12 h. Afterwards, brine (30 mL) was added, and the mixture was extracted by DCM (30 mL) for three times. The organic phase was collected, dried over anhydrous MgSO₄ and filtered. The filtrate was concentrated and purified by column chromatography (SiO₂) with hexane/EtOAc=20/1 as the eluent to give the title compound as a colorless oil (0.62 g, 79%).

### 3,5-Bis((2-ethylhexyl)oxy)benzyl 4-bromobutanoate (28b)

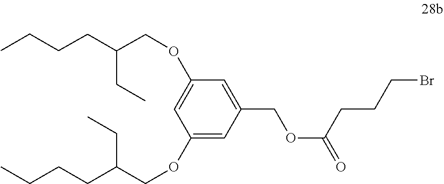 [SMILES: CCCCC(CC)COc1cc(COC(=O)CCCBr)cc(OCC(CC)CCCC)c1]

4-Bromobutyric acid (0.11 g, 0.63 mmol, 1.05 equiv) was dissolved in 5 mL anhydrous DCM and 1 drop of DMF was added. Thionyl chloride (SOCl₂, 0.14 g, 1.20 mmol, 2 equiv) was added and the mixture was stirred at 23° C. for 1 h. Afterwards, DCM and excess SOCl₂ was removed under reduced pressure to give the 4-bromobutanoyl chloride. Compound 8 (0.22 g, 0.60 mmol, 1.0 equiv), NEt₃ (67 mg, 0.66 mmol, 1.1 equiv) and DMAP (14 mg, 0.12 mmol, 0.2 equiv) were dissolved in 6 mL anhydrous DCM. To this mixture was added the DCM solution (3 mL) of above 4-bromobutanoyl chloride dropwise at 0° C. The mixture was allowed to warm to 23° C. with stirring during a 2 h period. Afterwards, water (30 mL) was added, and the mixture was extracted by DCM (30 mL) for three times. The organic phase was collected, dried over anhydrous MgSO₄ and filtered. The filtrate was concentrated and purified by column chromatography (SiO₂) with hexane/EtOAc=20/1 as the eluent to give the title compound as a colorless oil (0.26 g, 83%).

### 3,5-Bis(nonyloxy)benzyl 4-bromobutanoate (28c)

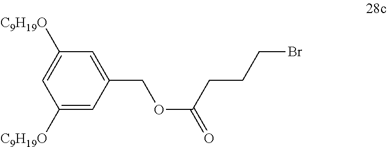

Compound 28c was synthesized from compound 2 (0.50 g, 1.30 mmol), 4-bromobutyric acid (0.22 g, 1.30 mmol) and NEt₃ (0.14 g, 1.43 mmol) following a procedure similar to that used for the synthesis of compound 28b. The title compound was obtained as a light-yellow oil (0.51 g, 74%).

### 3,4-Bis(dodecyloxy)benzyl 4-bromobutanoate (30a)

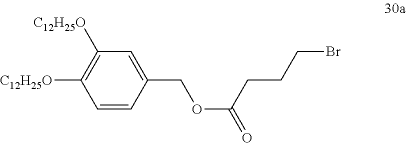

Compound 30a was synthesized from compound 9 (0.50 g, 1.05 mmol), 4-bromobutyric acid (0.18 g, 1.05 mmol) and NEt₃ (0.12 g, 1.16 mmol) following a procedure similar to that used for the synthesis of compound 28b. The title compound was obtained as a white solid (0.52 g, 79%).

### 3,4,5-Tris(dodecyloxy)benzyl 4-bromobutanoate (30b)

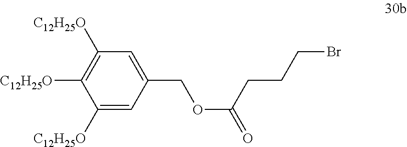

Compound 30b was synthesized from compound 11 (0.50 g, 0.76 mmol), 4-bromobutyric acid (0.13 g, 0.76 mmol) and NEt₃ (0.12 g, 0.84 mmol) following a procedure similar to that used for the synthesis of compound 28b. The title compound was obtained as a colorless oil (0.48 g, 79%).

### 3,5-Bis(dodecyloxy)benzyl 4-(4-(2-hydroxyethyl)piperazin-1-yl)butanoate (29a, IAJD71)

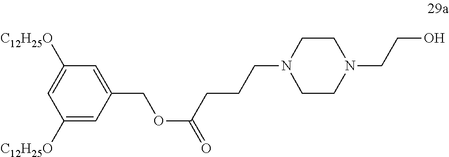

Compound 28a (0.24 g, 0.38 mmol, 1.0 equiv), 1-(2-hydroxyethyl)piperazine (52 mg, 0.40 mmol, 1.05 equiv), K₂CO₃ (55 mg, 0.40 mmol, 1.05 equiv) were stirred in MeCN (20 mL). The mixture was heated at reflux (95° C.) for 3 h. The reaction mixture was cooled to 23° C. and MeCN was removed under reduced pressure. Then water (20 mL) was added, and the mixture was extracted by DCM (20 mL×3). The organic phase was collected, dried over anhydrous MgSO₄, filtered and dried to give the crude product. The crude product was purified by column chromatography (SiO₂) with DCM/MeOH=30/1 and 15/1 as the eluent. Then the obtained product was dissolved in DCM (20 mL), which was washed by NaHCO₃solution (2%, 20 mL). The aqueous phase was extracted by DCM (20 mL) for another two times. The organic phase was combined and dried over anhydrous MgSO₄. Filtration and evaporation of the solvent yielded the title compound as a colorless oil (0.22 g, 85%).

### 3,5-Bis((2-ethylhexyl)oxy)benzyl 4-(4-methylpiperazin-1-yl)butanoate (29b, IAJD76)

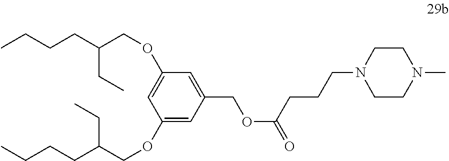 [SMILES: CCCCC(CC)COc1cc(COC(=O)CCCN2CCN(C)CC2)cc(OCC(CC)CCCC)c1]

Compound 29b was synthesized from compound 28b (0.26 g, 0.50 mmol), 1-methylpiperazine (56 mg, 0.56 mmol) and K₂CO₃ (0.10 g, 0.75 mmol) following a procedure similar to that used for the synthesis of compound 29a. The title compound was obtained as a colorless oil (0.19 g, 69%).

### 3,5-Bis(nonyloxy)benzyl 4-(4-methylpiperazin-1-yl)butanoate (29c, IAJD77)

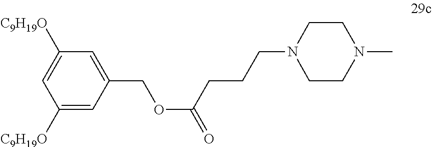

Compound 29c was synthesized from compound 28c (0.35 g, 0.64 mmol), 1-methylpiperazine (70 mg, 0.70 mmol) and K₂CO₃ (98 mg, 0.70 mmol) following a procedure similar to that used for the synthesis of compound 29a. The title compound was obtained as a light-yellow oil (0.26 g, 72%).

### 3,4-Bis(dodecyloxy)benzyl 4-(4-methylpiperazin-1-yl)butanoate (31a, IAJD78)

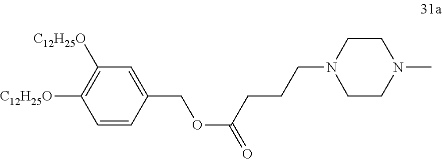

Compound 31a was synthesized from compound 30a (0.40 g, 0.64 mmol), 1-methylpiperazine (67 mg, 0.67 mmol) and K₂CO₃ (93 mg, 0.67 mmol) following a procedure similar to that used for the synthesis of compound 29a. The title compound was obtained as a viscous white solid (0.36 g, 88%).

### 3,4,5-Tris(dodecyloxy)benzyl 4-(4-methylpiperazin-1-yl)butanoate (31b, IAJD79)

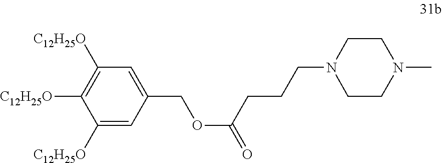

Compound 31b was synthesized from compound 30b (0.47 g, 0.58 mmol), 1-methylpiperazine (61 mg, 0.61 mmol) and K₂CO₃ (84 mg, 0.61 mmol) following a procedure similar to that used for the synthesis of compound 29a. The title compound was obtained as a light-yellow oil (0.41 g, 85%).

### 3,5-Bis(tetradecyloxy)benzyl 4-(4-methylpiperazin-1-yl)butanoate (32a, IAJD74)

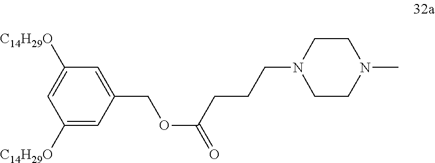

Compound 5 (0.24 g, 0.45 mmol, 1 equiv) and 4-(4-methylpiperazin-1-yl)butanoic acid hydrochloride (24b, 0.10 g, 0.45 mmol, 1 equiv) were dissolved in 6 mL dry DCM. NEt₃ (3 drops) was added. DCC (0.19 g, 0.90 mmol, 2 equiv) was added in one portion into the above mixture. The reaction was allowed to stir at 23° C. for 12 h. Afterwards, urea was removed by filtration, and washed with DCM carefully. The filtrate was concentrated and purified by column chromatography (SiO₂) with DCM/MeOH=40/1, 20/1 and 15/1 as the eluent. Then the product was dissolved in DCM (20 mL), which was washed by NaHCO₃solution (2%, 20 mL). The aqueous phase was extracted by DCM (20 mL) for another two times. The organic phase was combined and dried over anhydrous MgSO₄. Filtration and evaporation of the solvent yielded the title compound as a light-yellow oil (0.22 g, 71%).

### 3,5-Bis(hexadecyloxy)benzyl 4-(4-methylpiperazin-1-yl)butanoate (32b, IAJD75)

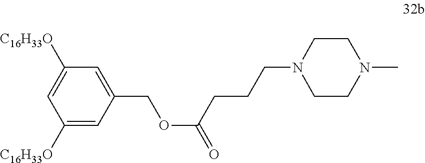

Compound 32b was synthesized from compound 6 (0.27 g, 0.45 mmol, 1 equiv), 4-(4-methylpiperazin-1-yl)butanoic acid hydrochloride (24b, 0.10 g, 0.45 mmol, 1 equiv) and DCC (0.19 g, 0.90 mmol, 2 equiv) following a procedure similar to that used for the synthesis of compound 32a. The title compound was obtained as a light-yellow oil (0.25 g, 73%).

Synthesis of IAJDs 81-86 was performed according to Scheme 6.

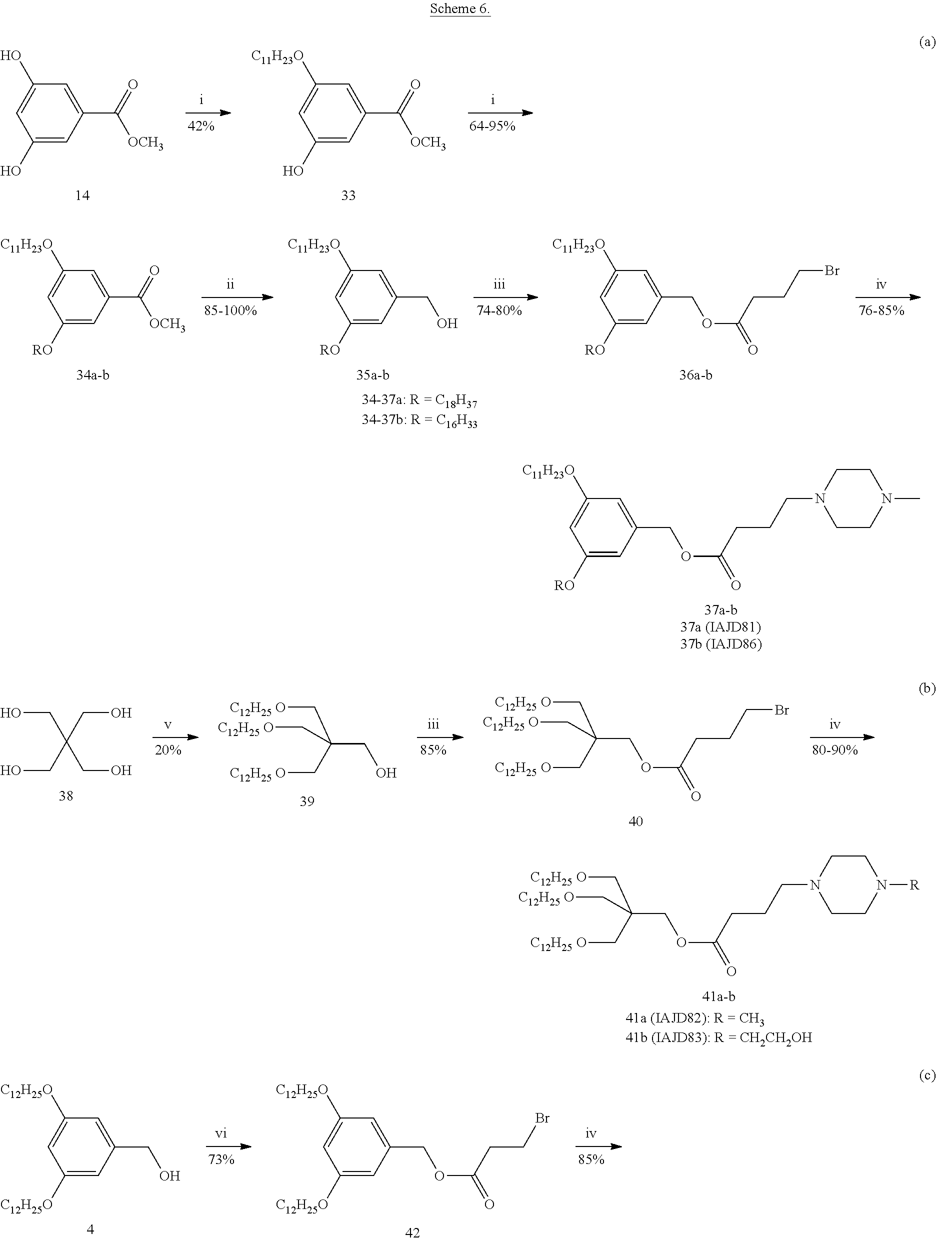

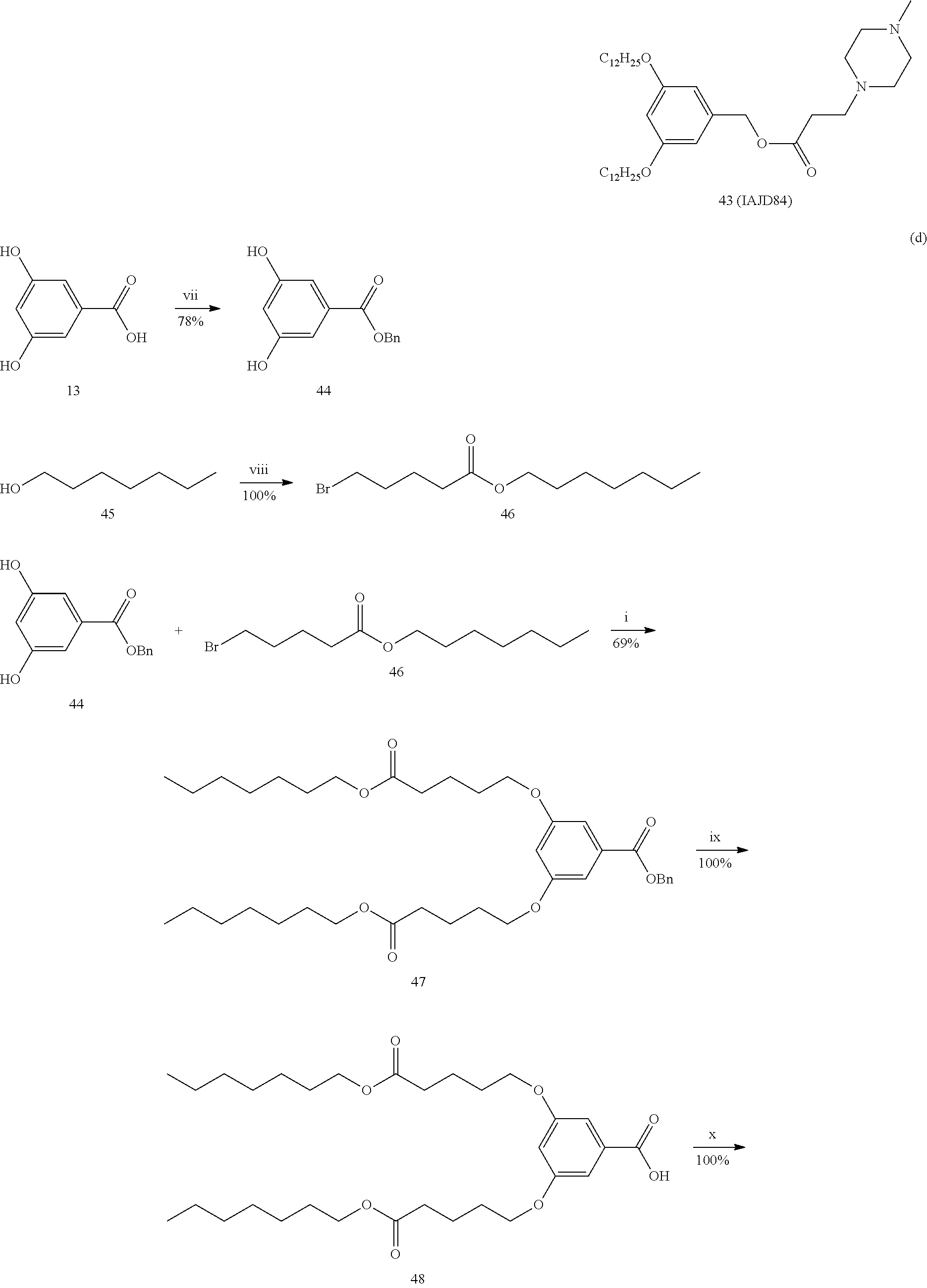

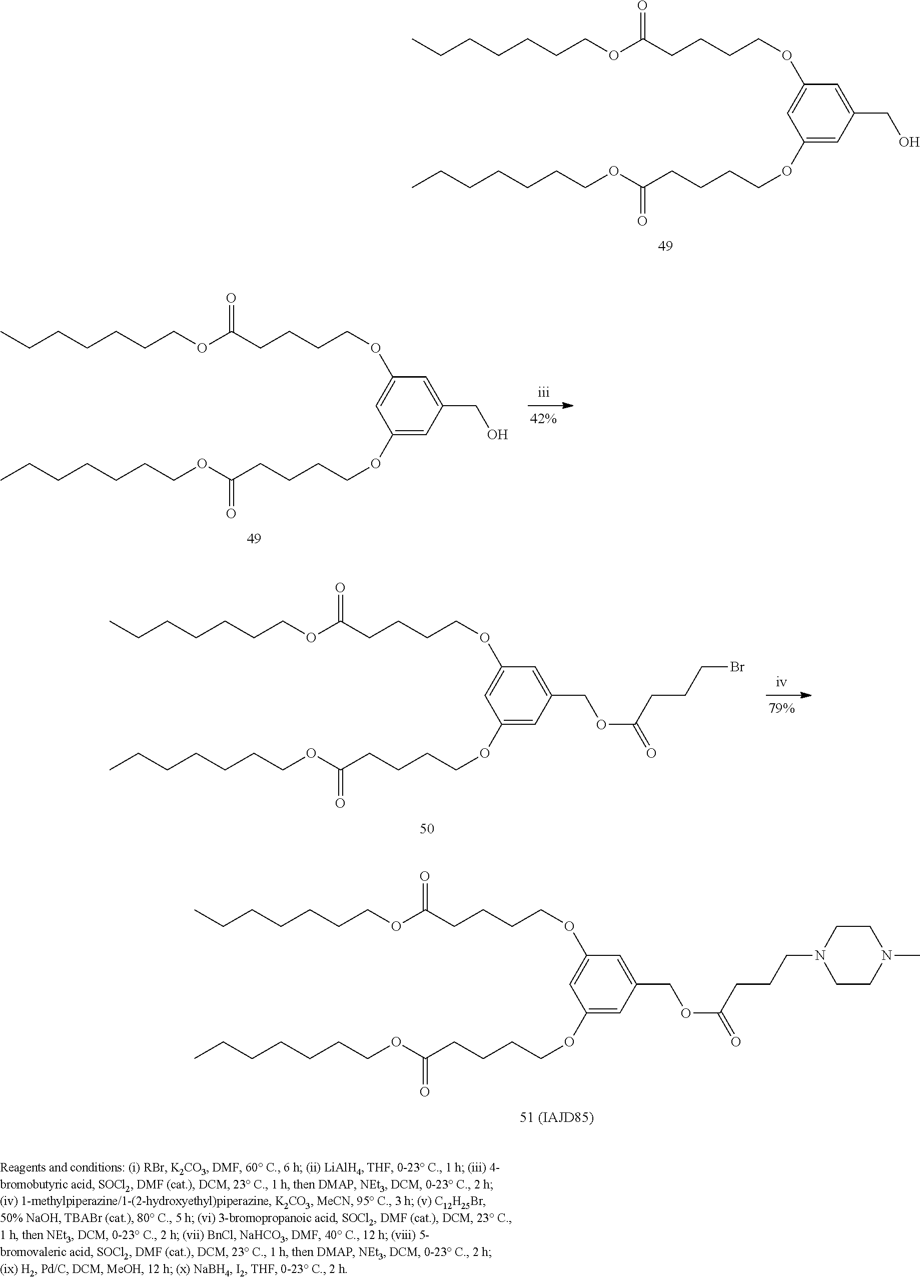 [SMILES: CCCCCCCOC(=O)CCCCOc1cc(CO)cc(OCCCCC(=O)OCCCCCCC)c1 ; CCCCCCCOC(=O)CCCCOc1cc(CO)cc(OCCCCC(=O)OCCCCCCC)c1 ; CCCCCCCOC(=O)CCCCOc1cc(COC(=O)CCCBr)cc(OCCCCC(=O)OCCCCCCC)c1 ; CCCCCCCOC(=O)CCCCOc1cc(COC(=O)CCCN2CCN(C)CC2)cc(OCCCCC(=O)OCCCCCCC)c1]

### Methyl 3-hydroxy-5-(undecyloxy)benzoate (33)

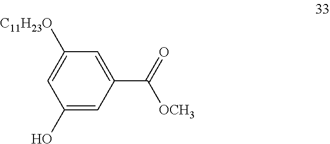

Methyl 3,5-dihydroxybenzoate (14, 5.00 g, 29.7 mmol, 1.2 equiv), 1-bromoundecane (5.83 g, 24.8 mmol, 1 equiv), and K₂CO₃ (3.42 g, 24.8 mmol, 1 equiv) were stirred in DMF (50 mL). The mixture was stirred at 60° C. under N₂ atmosphere for 6 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with hexane/EtOAc=10/1 as the eluent to give the title compound as a white solid (3.31g, 42%).

### Methyl 3-(octadecyloxy)-5-(undecyloxy)benzoate (34a)

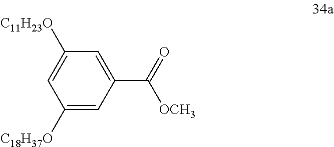

Compound 34a was synthesized from compound 33 (1.0 g, 3.10 mmol, 1 equiv), 1-bromooctadecane (1.24 g, 3.72 mmol, 1.2 equiv), and K₂CO₃ (1.28 g, 9.30 mmol, 3 equiv) following a procedure similar to that used for the synthesis of compound 33. The title compound was obtained as a white solid (1.69 g, 95%).

### Methyl 3-(hexadecyloxy)-5-(undecyloxy)benzoate (34b)

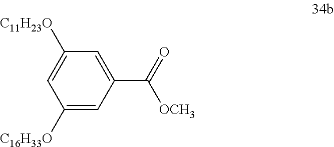

Compound 34b was synthesized from compound 33 (0.50 g, 1.55 mmol, 1 equiv), 1-bromohexadecane (0.52 g, 1.71 mmol, 1.1 equiv), and K₂CO₃ (0.64 g, 4.65 mmol, 3 equiv) following a procedure similar to that used for the synthesis of compound 33. The title compound was obtained as a white solid (0.54 g, 64%).

### (3-(Octadecyloxy)-5-(undecyloxy)phenyl)methanol (35a)

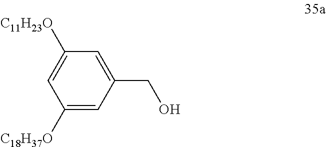

Compound 34a (1.0 g, 1.74 mmol, 1 equiv) was dissolved in 5 mL dry THF, which was added dropwise to a slurry of LiAlH₄ (0.10 g, 2.61 mmol, 1.5 equiv) in dry THF (5 mL) at 0° C. under N₂ atmosphere. The resulted mixture was stirred at 23° C. for 1 h. The reaction was quenched by the successive addition of water (0.3 mL), 15% NaOH aqueous solution (0.3 mL) and water (1.5 mL). Then the mixture was filtered to remove white precipitates and the filtrate was dried over anhydrous MgSO₄. Filtration and evaporation of the solvent yielded the title compound as a white solid (0.95 g, 100%).

### (3-(Hexadecyloxy)-5-(undecyloxy)phenyl)methanol (35b)

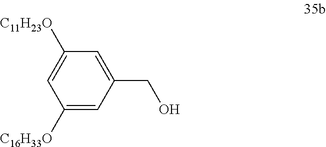

Compound 35b was synthesized from compound 34b (0.50 g, 0.91 mmol, 1 equiv) and LiAlH₄ (52 mg, 1.37 mmol, 1.5 equiv) following a procedure similar to that used for the synthesis of compound 35a. The title compound was obtained as a white solid (0.40 g, 85%).

### 3-(Octadecyloxy)-5-(undecyloxy)benzyl 4-bromobutanoate (36a)

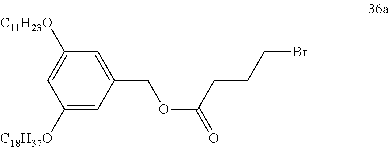

4-Bromobutyric acid (0.10 g, 0.60 mmol, 1.0 equiv) was dissolved in 5 mL anhydrous DCM and 1 drop of DMF was added. SOCl₂ (0.14 g, 1.20 mmol, 2 equiv) was added and the mixture was stirred at 23° C. for 1 h. Afterwards, DCM and excess SOCl₂ was removed under reduced pressure to give the 4-bromobutanoyl chloride. Compound 35a (0.35 g, 0.60 mmol, 1 equiv) and NEt₃ (0.12 g, 1.20 mmol, 2 equiv) were dissolved in 6 mL anhydrous DCM. Then DMAP (15 mg, 0.12 mmol, 0.2 equiv) was added. To this mixture was added the DCM solution (3 mL) of above 4-bromobutanoyl chloride dropwise at 0° C. The mixture was allowed to warm to 23° C. with stirring during a 2 h period. Afterwards, water (30 mL) was added, and the mixture was extracted by DCM (30 mL) for three times. The organic phase was collected, dried over anhydrous MgSO₄ and filtered. The filtrate was concentrated and purified by column chromatography (SiO₂) with hexane/EtOAc=20/1 as the eluent to give the title compound as a colorless oil (0.31 g, 74%).

### 3-(Hexadecyloxy)-5-(undecyloxy)benzyl 4-bromobutanoate (36b)

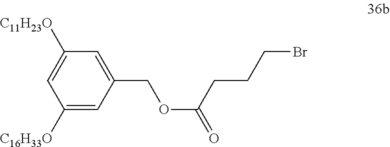

Compound 36b was synthesized from compound 35b (0.37 g, 0.60 mmol, 1 equiv), 4-bromobutyric acid (0.10 g, 0.60 mmol, 1.0 equiv), NEt₃ (0.12 g, 1.20 mmol, 2 equiv) and DMAP (15 mg, 0.12 mmol, 0.2 equiv) following a procedure similar to that used for the synthesis of compound 36a. The title compound was obtained as a colorless oil (0.33 g, 80%).

### 3-(Octadecyloxy)-5-(undecyloxy)benzyl 4-(4-methylpiperazin-1-yl)butanoate (37a, IAJD81)

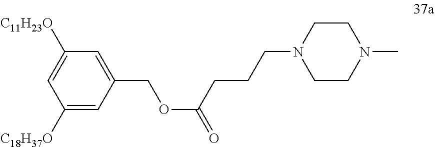

Compound 36a (0.24 g, 0.35 mmol, 1 equiv), 1-methylpiperazine (37 mg, 0.36 mmol, 1.05 equiv), K₂CO₃ (53 mg, 0.38 mmol, 1.1 equiv) were stirred in MeCN (20 mL). The mixture was heated at reflux (95° C.) for 3 h. The reaction mixture was cooled to 23° C. and MeCN was removed under reduced pressure. Then water (20 mL) was added, and the mixture was extracted by DCM (20 mL×3). The organic phase was collected, dried over anhydrous MgSO₄, filtered and dried to give the crude product. The crude product was purified by column chromatography (SiO₂) with DCM/MeOH=30/1 and 15/1 as the eluent. Then the obtained product was dissolved in DCM (20 mL), which was washed by NaHCO₃solution (2%, 20 mL). The aqueous phase was extracted by DCM (20 mL) for another two times. The organic phase was combined and dried over anhydrous MgSO₄. Filtration and evaporation of the solvent yielded the title compound as a light-yellow oil (0.21 g, 85%).

### 3-(Hexadecyloxy)-5-(undecyloxy)benzyl 4-(4-methylpiperazin-1-yl)butanoate (37b, IAJD86)

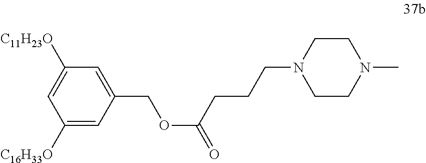

Compound 37b was synthesized from compound 36b (0.33 g, 0.49 mmol, 1 equiv), 1-methylpiperazine (52 mg, 0.52 mmol, 1.05 equiv) and K₂CO₃ (74 mg, 0.53 mmol, 1.1 equiv) following a procedure similar to that used for the synthesis of compound 37a. The title compound was obtained as a light-yellow oil (0.31 g, 76%).

### 3-(Dodecyloxy)-2,2-bis((dodecyloxy)methyl)propyl 4-bromobutanoate (40)

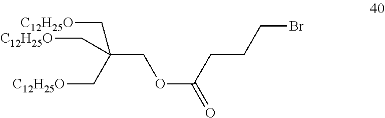

The synthesis and characterizations of 3-(dodecyloxy)-2,2-bis((dodecyloxy)methyl)propan-1-ol (39) were reported in the literature.⁵ 4-Bromobutyric acid (0.15 g, 0.86 mmol, 1.1 equiv) was dissolved in 5 mL anhydrous DCM and 1 drop of DMF was added. SOCl₂ (0.19 g, 1.56 mmol, 2 equiv) was added and the mixture was stirred at 23° C. for 1 h. Afterwards, DCM and excess SOCl₂ was removed under reduced pressure to give the 4-bromobutanoyl chloride. Compound 39 (0.50 g, 0.78 mmol, 1.0 equiv) and NEt₃ (87 mg, 0.86 mmol, 1.1 equiv) were dissolved in 6 mL anhydrous DCM. To this mixture was added the DCM solution (3 mL) of above 4-bromobutanoyl chloride dropwise at 0° C. The mixture was allowed to warm to 23° C. with stirring during a 2 h period. Afterwards, water (20 mL) was added, and the mixture was extracted by DCM (20 mL) for three times. The organic phase was collected, dried over anhydrous MgSO₄ and filtered. The filtrate was concentrated and purified by column chromatography (SiO₂) with hexane/EtOAc=40/1 as the eluent to give the title compound as a colorless oil (0.48 g, 85%).

### 3-(Dodecyloxy)-2,2-bis((dodecyloxy)methyl)propyl 4-(4-methylpiperazin-1-yl)butanoate (41a, IAJD82)

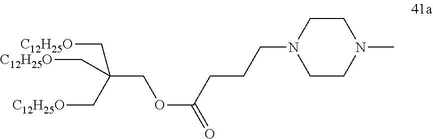

Compound 40 (0.15 g, 0.19 mmol, 1 equiv), 1-methylpiperazine (26 mg, 0.20 mmol, 1.05 equiv), K₂CO₃ (26 mg, 0.20 mmol, 1.05 equiv) were stirred in MeCN (15 mL). The mixture was heated at reflux (95° C.) for 3 h. The reaction mixture was cooled to 23° C. and MeCN was removed under reduced pressure. Then water (20 mL) was added, and the mixture was extracted by DCM (20 mL×3). The organic phase was collected, dried over anhydrous MgSO₄, filtered and dried to give the crude product. The crude product was purified by column chromatography (SiO₂) with DCM/MeOH=30/1 and 20/1 as the eluent. Then the obtained product was dissolved in DCM (20 mL), which was washed by NaHCO₃solution (2%, 20 mL). The aqueous phase was extracted by DCM (20 mL) for another two times. The organic phase was combined and dried over anhydrous MgSO₄. Filtration and evaporation of the solvent yielded the title compound as a light-yellow oil (0.13 g, 80%).

### 3-(Dodecyloxy)-2,2-bis((dodecyloxy)methyl)propyl 4-(4-(2-hydroxyethyl)piperazin-1-yl)butanoate (41b, IAJD83)

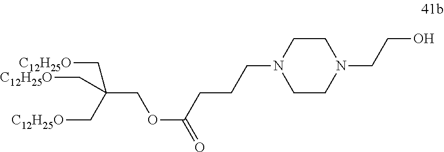

Compound 41b was synthesized from compound 40 (0.19 g, 0.24 mmol, 1 equiv), 1-(2-hydroxyethyl)piperazine (33 mg, 0.25 mmol, 1.05 equiv) and K₂CO₃ (33 mg, 0.25 mmol, 1.05 equiv) following a procedure similar to that used for the synthesis of compound 41a. The title compound was obtained as a light-yellow oil (0.18 g, 90%).

### 3,5-Bis(dodecyloxy)benzyl 3-bromopropanoate (42)

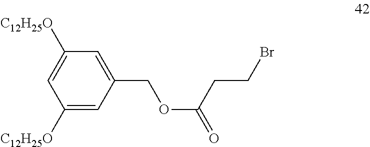

3-Bromopropanoic acid (0.16 g, 1.05 mmol, 1.0 equiv) was dissolved in 5 mL anhydrous DCM and 1 drop of DMF was added. SOCl₂ (0.25 g, 2.10 mmol, 2.0 equiv) was added and the mixture was stirred at 23° C. for 1 h. Afterwards, DCM and excess SOCl₂ was removed under reduced pressure to give the 3-bromopropanoyl chloride. Compound 4 (0.50 g, 1.05 mmol, 1.0 equiv) and NEt₃ (0.12 g, 1.16 mmol, 1.1 equiv) were dissolved in 6 mL anhydrous DCM. To this mixture was added the DCM solution (3 mL) of above 3-bromopropanoyl chloride dropwise at 0° C. The mixture was allowed to warm to 23° C. with stirring during a 2 h period. Afterwards, water (30 mL) was added, and the mixture was extracted by DCM (30 mL) for three times. The organic phase was collected, dried over anhydrous MgSO₄ and filtered. The filtrate was concentrated and purified by column chromatography (SiO₂) with hexane/EtOAc=20/1 as the eluent to give the title compound as a colorless oil (0.47 g, 73%).

### 3,5-Bis(dodecyloxy)benzyl 3-(4-methylpiperazin-1-yl)propanoate (43, IAJD84)

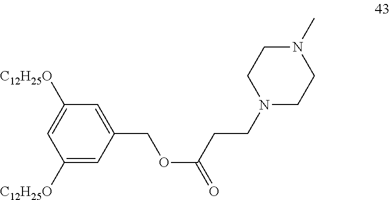

Compound 42 (0.26 g, 0.43 mmol, 1 equiv), 1-methylpiperazine (45 mg, 0.45 mmol, 1.05 equiv), K₂CO₃ (62 mg, 0.45 mmol, 1.05 equiv) were stirred in MeCN (15 mL). The mixture was heated at reflux (95° C.) for 3 h. The reaction mixture was cooled to 23° C. and MeCN was removed under reduced pressure. Then water (20 mL) was added, and the mixture was extracted by DCM (20 mL×3). The organic phase was collected, dried over anhydrous MgSO₄, filtered and dried to give the crude product. The crude product was purified by column chromatography (SiO₂) with DCM/MeOH=40/1, 20/1 and 15/1 as the eluent. Then the obtained product was dissolved in DCM (20 mL), which was washed by NaHCO₃solution (2%, 20 mL). The aqueous phase was extracted by DCM (20 mL) for another two times. The organic phase was combined and dried over anhydrous MgSO₄. Filtration and evaporation of the solvent yielded the title compound as a light-yellow oil (0.23 g, 85%).

### Benzyl 3,5-dihydroxybenzoate (44)

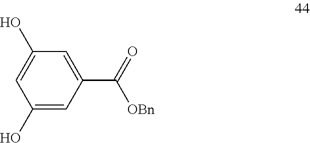 [SMILES: O=C(OCc1ccccc1)c1cc(O)cc(O)c1]

The synthesis of compound 44 was adapted from literature procedure.⁸ 3,5-Dihydroxyl benzoic acid (13, 5.0 g, 32.4 mmol), benzyl chloride (6.1 g, 48.5 mmol) and NaHCO₃(3.3 g, 38.9 mmol) were mixed with DMF (60 mL), and allowed to stir at 40° C. for 12 h. After the reaction mixture was cooled to 23° C., DCM (350 mL) and saturated NH₄Cl aqueous solution (350 mL) was added. The mixture was extracted with DCM (100 mL) for 3 times. The organic extract was dried over anhydrous MgSO₄, filtered and dried to give the crude product. The crude product was further purified by column chromatography (SiO₂) with a mobile phase of hexane/EtOAc=3/2 (v/v) to yield the title compound as a white solid (6.2 g, 78%).

### Heptyl 5-bromopentanoate (46)

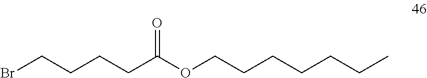

To a solution of 5-bromovaleric acid (5.00 g, 27.6 mmol) in dry DCM (20 mL) was added 1 drop of DMF, followed by the dropwise addition of SOCl₂ (6.57 g, 55.2 mmol). The reaction mixture was stirred at 23° C. for 30 min, then DCM and excess SOCl₂ was removed under reduced pressure. The obtained acyl chloride was dissolved in dry DCM (10 mL) and added dropwise into a solution of 1-heptanol (3.55 g, 30.4 mmol) and NEt₃ (3.07 g, 30.4 mmol) in dry DCM (20 mL) at 0° C. After the mixture was stirred at 23° C. for 12 h, water (50 mL) was added, and the mixture was extracted by DCM (50 mL×3). The organic phase was dried over anhydrous MgSO₄ and filtered. The filtrate was concentrated and purified by column chromatography (SiO₂) with hexane/EtOAc=10/1 as the eluent to give the title compound as a colorless oil (7.70 g, 100%).

### Diheptyl 5,5′-((5-((benzyloxy)carbonyl)-1,3-phenylene)bis(oxy))dipentanoate (47)

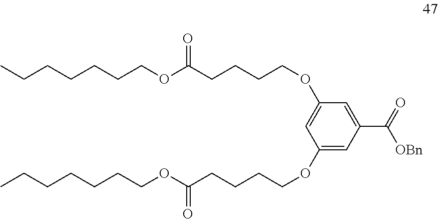 [SMILES: CCCCCCCOC(=O)CCCCOc1cc(OCCCCC(=O)OCCCCCCC)cc(C(=O)OCc2ccccc2)c1]

Compound 44 (0.39 g, 1.58 mmol), compound 46 (1.10 g, 4.00 mmol), and K₂CO₃ (1.30 g, 9.50 mmol) were stirred in DMF (25 mL). The mixture was stirred at 60° C. under N₂ atmosphere for 6 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with hexane/EtOAc=20/1 as the eluent to give the title compound as a colorless oil (0.70 g, 69%).

### 3,5-Bis((5-(heptyloxy)-5-oxopentyl)oxy)benzoic acid (48)

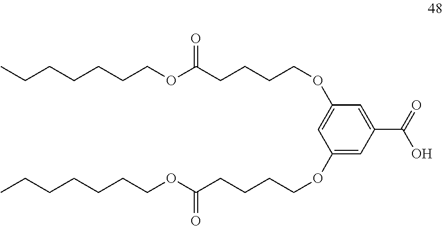 [SMILES: CCCCCCCOC(=O)CCCCOc1cc(OCCCCC(=O)OCCCCCCC)cc(C(=O)O)c1]

Compound 47 (0.70 g, 1.09 mmol) was dissolved in 1:2 MeOH:DCM (30 mL). Then Pd/C (50 mg, 7 wt %) was added and the flask was evacuated and filled with hydrogen for three times. The mixture was stirred at 23° C. for 12 h under hydrogen atmosphere. The reaction mixture was filtered through Celite and the filter cake was washed with DCM. Evaporation of the solvent yielded the title compound as a colorless oil (0.60 g, 100%).

### Diheptyl 5,5′-((5-(hydroxymethyl)-1,3-phenylene)bis(oxy))dipentanoate (49)

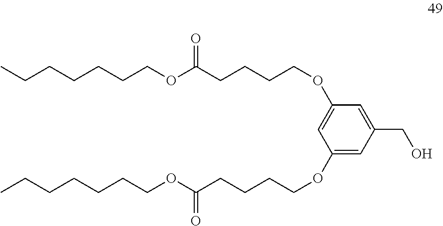 [SMILES: CCCCCCCOC(=O)CCCCOc1cc(CO)cc(OCCCCC(=O)OCCCCCCC)c1]

The synthesis of compound 49 was adapted from literature procedure.⁹ To a solution of compound 48 (0.60 g, 1.09 mmol) in 30 mL THF was added NaBH₄ (0.10 g, 2.75 mmol). The reaction mixture was stirred at 23° C. for 30 min under N₂ until no more gas was generated. A solution of iodine (0.34 g, 1.32 mmol) in THF (10 mL) was added dropwise into the reaction mixture under N₂ in 20 min at 0° C. After stirring at 0° C. for 30 min, the reaction was returned to 23° C. and stirred for 1.5 h. The reaction was quenched with HCl (2 M) solution (10 mL) until no more gas was generated, and then saturated NaHCO₃aqueous solution was added until pH=7. The mixture was extracted by DCM (50 mL×3). The organic phase was dried over anhydrous MgSO₄ and filtered. The alcohol as the product (0.59 g, 100%) was obtained by concentrating the filtrate as colorless oil without further purification.

### Diheptyl 5,5′-((5-(((4-bromobutanoyl)oxy)methyl)-1,3-phenylene)bis(oxy))dipentanoate (50)

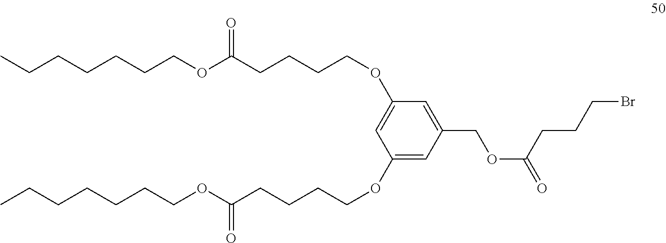 [SMILES: CCCCCCCOC(=O)CCCCOc1cc(COC(=O)CCCBr)cc(OCCCCC(=O)OCCCCCCC)c1]

4-Bromobutyric acid (0.22 g, 1.30 mmol, 1.2 equiv) was dissolved in 5 mL anhydrous DCM and 1 drop of DMF was added. SOCl₂ (0.26 g, 2.20 mmol, 2.0 equiv) was added and the mixture was stirred at 23° C. for 1 h. Afterwards, DCM and excess SOCl₂ was removed under reduced pressure to give the 4-bromobutanoyl chloride. Compound 49 (0.59 g, 1.10 mmol, 1.0 equiv) and NEt₃ (0.13 g, 1.30 mmol, 1.2 equiv) were dissolved in 6 mL anhydrous DCM. Then DMAP (24 mg, 0.20 mmol, 0.2 equiv) was added. To this mixture was added the DCM solution (3 mL) of above 4-bromobutanoyl chloride dropwise at 0° C. The mixture was allowed to warm to 23° C. with stirring during a 2 h period. Afterwards, water (20 mL) was added, and the mixture was extracted by DCM (20 mL) for three times. The organic phase was collected, dried over anhydrous MgSO₄ and filtered. The filtrate was concentrated and purified by column chromatography (SiO₂) with hexane/EtOAc=10/1 as the eluent to give the title compound as a colorless oil (0.32 g, 42%).

### Diheptyl 5,5′-((5-(((4-(4-methylpiperazin-1-yl)butanoyl)oxy)methyl)-1,3-phenylene)bis(oxy)) dipentanoate (51, IAJD85)

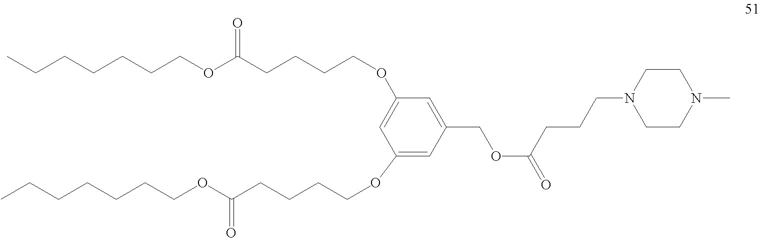 [SMILES: CCCCCCCOC(=O)CCCCOc1cc(COC(=O)CCCN2CCN(C)CC2)cc(OCCCCC(=O)OCCCCCCC)c1]

Compound 50 (0.32 g, 0.47 mmol, 1 equiv), 1-methylpiperazine (50 mg, 0.50 mmol, 1.05 equiv), K₂CO₃ (69 mg, 0.50 mmol, 1.05 equiv) were stirred in MeCN (15 mL). The mixture was heated at reflux (95° C.) for 3 h. The reaction mixture was cooled to 23° C. and MeCN was removed under reduced pressure. Then water (20 mL) was added, and the mixture was extracted by DCM (20 mL×3). The organic phase was collected, dried over anhydrous MgSO₄, filtered and dried to give the crude product. The crude product was purified by column chromatography (SiO₂) with DCM/MeOH=40/1, 20/1 and 10/1 as the eluent. Then the obtained product was dissolved in DCM (20 mL), which was washed by NaHCO₃solution (2%, 20 mL). The aqueous phase was extracted by DCM (20 mL) for another two times. The organic phase was combined and dried over anhydrous MgSO₄. Filtration and evaporation of the solvent yielded the title compound as a colorless oil (0.28 g, 79%). 4.6 Synthesis of IAJDs 87-89, 91 and 95 was performed according to Scheme 7

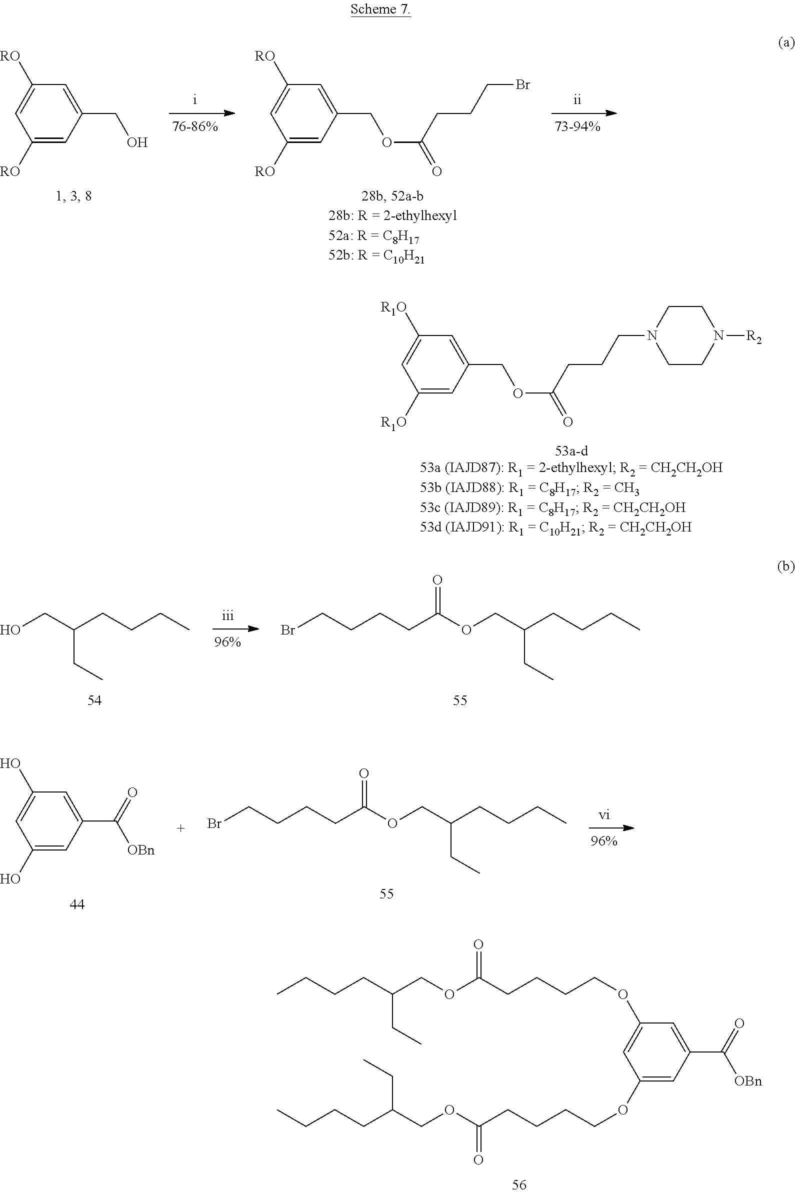

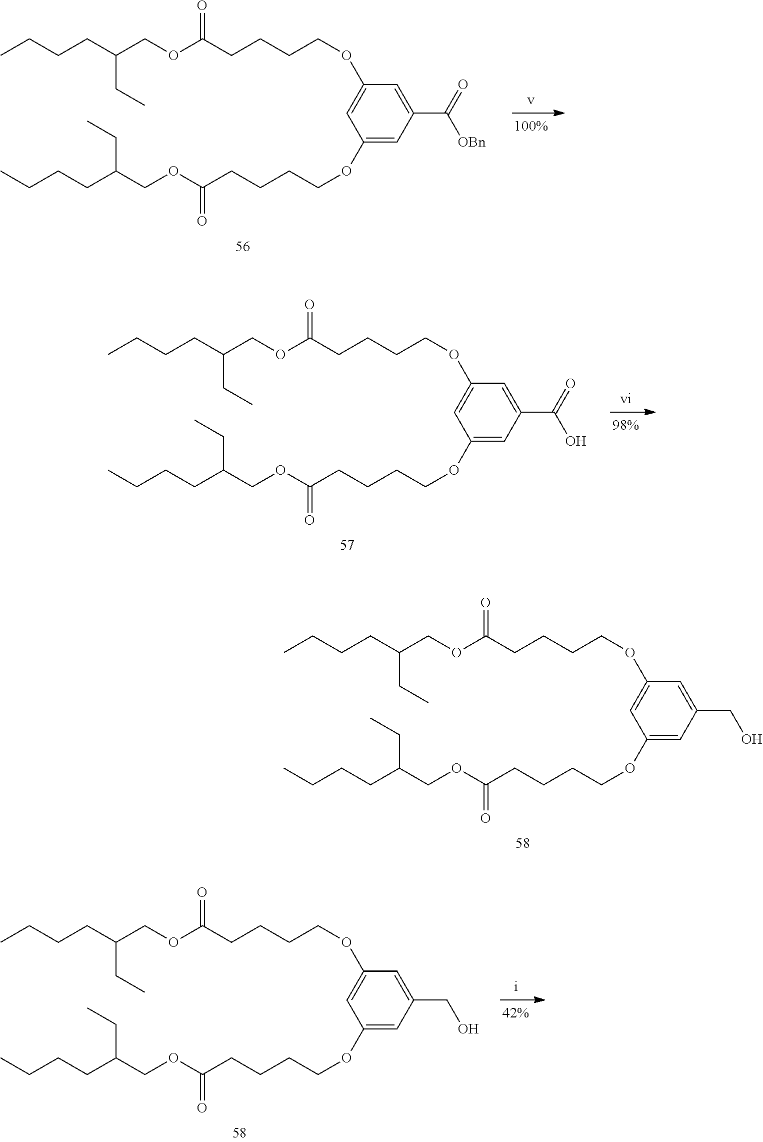 [SMILES: CCCCC(CC)COC(=O)CCCCOc1cc(OCCCCC(=O)OCC(CC)CCCC)cc(C(=O)OCc2ccccc2)c1 ; CCCCC(CC)COC(=O)CCCCOc1cc(OCCCCC(=O)OCC(CC)CCCC)cc(C(=O)O)c1 ; CCCCC(CC)COC(=O)CCCCOc1cc(CO)cc(OCCCCC(=O)OCC(CC)CCCC)c1 ; CCCCC(CC)COC(=O)CCCCOc1cc(CO)cc(OCCCCC(=O)OCC(CC)CCCC)c1]

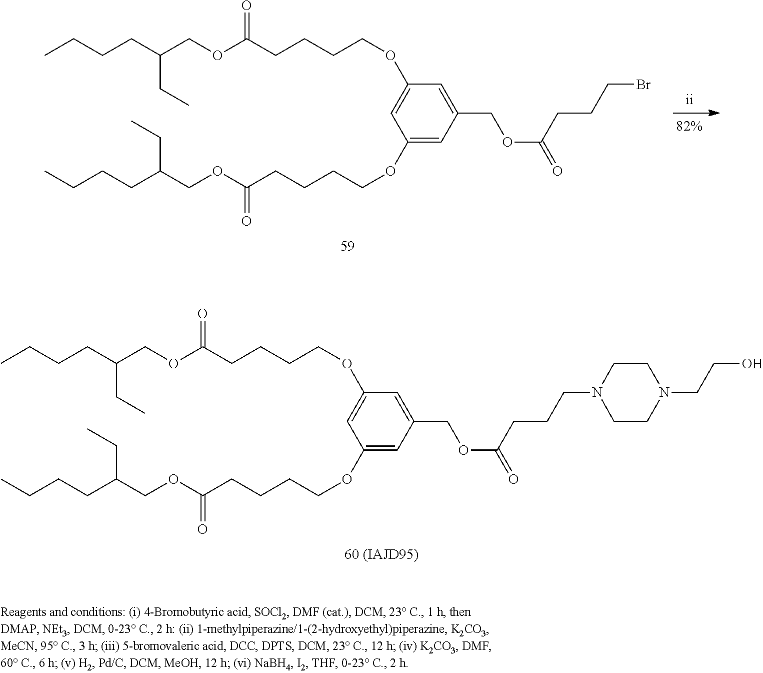 [SMILES: CCCCC(CC)COC(=O)CCCCOc1cc(COC(=O)CCCBr)cc(OCCCCC(=O)OCC(CC)CCCC)c1 ; CCCCC(CC)COC(=O)CCCCOc1cc(COC(=O)CCCN2CCN(CCO)CC2)cc(OCCCCC(=O)OCC(CC)CCCC)c1]

3,5-Bis(octyloxy)benzyl 4-bromobutanoate (52a).

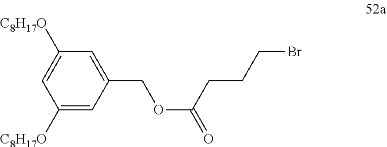

4-Bromobutyric acid (0.45 g, 2.70 mmol, 1.0 equiv) was dissolved in 6 mL anhydrous DCM and 1 drop of DMF was added. SOCl₂ (0.64 g, 5.40 mmol, 2.0 equiv) was added and the mixture was stirred at 23° C. for 1 h. Afterwards, DCM and excess SOCl₂ was removed under reduced pressure to give the 4-bromobutanoyl chloride. Compound 1 (1.00 g, 2.70 mmol, 1.0 equiv) and NEt₃ (0.30 g, 3.00 mmol, 1.1 equiv) were dissolved in 10 mL anhydrous DCM. To this mixture was added the DCM solution (5 mL) of above 4-bromobutanoyl chloride dropwise at 0° C. The mixture was allowed to warm to 23° C. with stirring during a 2 h period. Afterwards, water (30 mL) was added, and the mixture was extracted by DCM (30 mL) for three times. The organic phase was collected, dried over anhydrous MgSO₄ and filtered. The filtrate was concentrated and purified by column chromatography (SiO₂) with hexane/EtOAc=20/1 as the eluent to give the title compound as a light-yellow oil (1.22 g, 86%).

### 3,5-Bis(decyloxy)benzyl 4-bromobutanoate (52b)

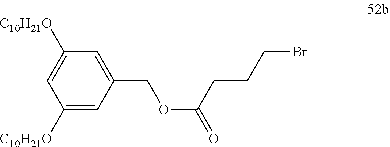

Compound 52b was synthesized from compound 3 (1.00 g, 2.38 mmol, 1.0 equiv), 4-bromobutyric acid (0.40 g, 2.38 mmol, 1.0 equiv) and NEt₃ (0.27 g, 2.62 mmol, 1.1 equiv) following a procedure similar to that used for the synthesis of compound 52a. The title compound was obtained as a light-yellow oil (1.02 g, 76%).

### 3,5-Bis((2-ethylhexyl)oxy)benzyl 4-(4-(2-hydroxyethyl)piperazin-1-yl)butanoate (53a, IAJD87)

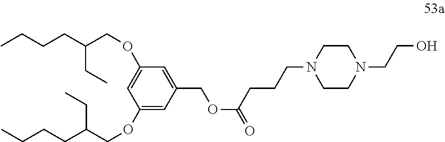 [SMILES: CCCCC(CC)COc1cc(COC(=O)CCCN2CCN(CCO)CC2)cc(OCC(CC)CCCC)c1]

Compound 28b (0.30 g, 0.58 mmol, 1.0 equiv), 1-(2-hydroxyethyl)piperazine (79 mg, 0.61 mmol, 1.05 equiv), K₂CO₃ (84 mg, 0.61 mmol, 1.05 equiv) were stirred in MeCN (20 mL). The mixture was heated at reflux (95° C.) for 3 h. The reaction mixture was cooled to 23° C. and MeCN was removed under reduced pressure. Then water (20 mL) was added, and the mixture was extracted by DCM (20 mL×3). The organic phase was collected, dried over anhydrous MgSO₄, filtered and dried to give the crude product. The crude product was purified by column chromatography (SiO₂) with DCM/MeOH=40/1 and 15/1 as the eluent. Then the obtained product was dissolved in DCM (20 mL), which was washed by NaHCO₃solution (2%, 20 mL). The aqueous phase was extracted by DCM (20 mL) for another two times. The organic phase was combined and dried over anhydrous MgSO₄. Filtration and evaporation of the solvent yielded the title compound as a light-yellow oil (0.31 g, 94%).

### 3,5-Bis(octyloxy)benzyl 4-(4-methylpiperazin-1-yl)butanoate (53b, IAJD88)

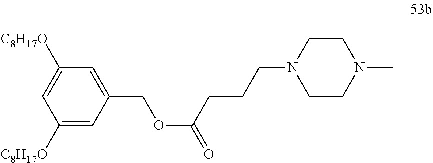

Compound 53b was synthesized from compound 52a (0.30 g, 0.58 mmol, 1 equiv), 1-methylpiperazine (61 mg, 0.61 mmol, 1.05 equiv) and K₂CO₃ (85 mg, 0.61 mmol, 1.05 equiv) following a procedure similar to that used for the synthesis of compound 53a. The title compound was obtained as a light-yellow oil (0.23 g, 74%).

### 3,5-Bis(octyloxy)benzyl 4-(4-(2-hydroxyethyl piperazin-1-yl)butanoate (53c, IAJD89)

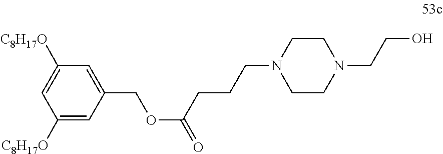

Compound 53c was synthesized from compound 52a (0.30 g, 0.58 mmol, 1 equiv), 1-(2-hydroxyethyl)piperazine (82 mg, 0.61 mmol, 1.05 equiv) and K₂CO₃ (85 mg, 0.61 mmol, 1.05 equiv) following a procedure similar to that used for the synthesis of compound 53a. The title compound was obtained as a light-yellow oil (0.24 g, 73%).

### 3,5-Bis(decyloxy)benzyl 4-(4-(2-hydroxyethyl)piperazin-1-yl)butanoate (53d, IAJD91)

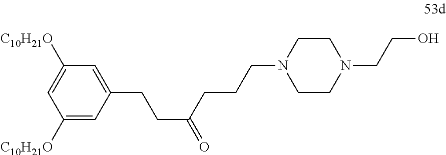

Compound 53d was synthesized from compound 52b (0.30 g, 0.53 mmol, 1 equiv), 1-(2-hydroxyethyl)piperazine (73 mg, 0.56 mmol, 1.05 equiv) and K₂CO₃ (77 mg, 0.56 mmol, 1.05 equiv) following a procedure similar to that used for the synthesis of compound 53a. The title compound was obtained as a colorless oil (0.31 g, 94%).

### 2-Ethylhexyl 5-bromopentanoate (55)

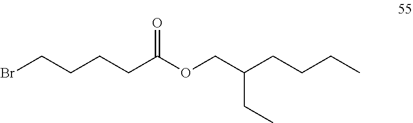 [SMILES: CCCCC(CC)COC(=O)CCCCBr]

20 To a DCM (10 mL) solution of 2-ethylhexanol (54, 2.00 g, 11.1 mmol), 5-bromovaleric acid (1.44 g, 11.1 mmol) and DPTS (3.60 g, 12.2 mmol), was added DCC (4.58 g, 22.2 mmol). The mixture was allowed to stir at 23° C. for 12 h. The precipitate was filtered, and the filtrate was concentrated to dryness. The crude product was further purified by column chromatography with a mobile phase of hexane/DCM=1/1 to yield the title compound as a colorless oil (3.13 g, 96%).

### Bis(2-ethylhexyl) 5,5′-((5-((benzyloxy)carbonyl)-1,3-phenylene)bis(oxy))dipentanoate (56)

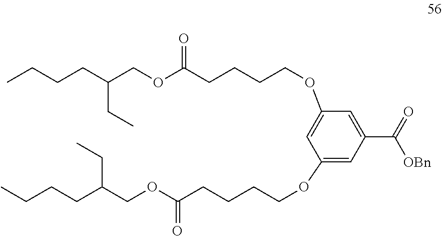 [SMILES: CCCCC(CC)COC(=O)CCCCOc1cc(OCCCCC(=O)OCC(CC)CCCC)cc(C(=O)OCc2ccccc2)c1]

Benzyl 3,5-hydroxyl benzoate (44, 0.78 g, 3.21 mmol), compound 55 (2.00 g, 6.75 mmol) and K₂CO₃ (2.66 g, 19.3 mmol) were stirred in DMF (20 mL). The mixture was stirred at 60° C. under N₂ atmosphere for 6 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with DCM as the eluent to give the title compound as a colorless oil (2.95 g, 96%).

### 3,5-Bis((5-((2-ethylhexyl)oxy)-5-oxopentyl)oxy)benzoic acid (57)

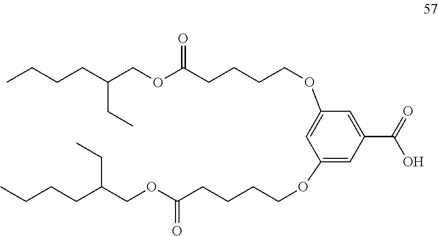 [SMILES: CCCCC(CC)COC(=O)CCCCOc1cc(OCCCCC(=O)OCC(CC)CCCC)cc(C(=O)O)c1]

Compound 56 (2.13 g, 3.18 mmol) was dissolved in 1:2 MeOH:DCM (45 mL). Then Pd/C (0.21 g, 10 wt %) was added and the flask was evacuated and filled with hydrogen for three times. The mixture was stirred at 23° C. for 12 h under hydrogen atmosphere. The reaction mixture was filtered through Celite and the filter cake was washed with DCM. Evaporation of the solvent yielded the title compound as a colorless oil (1.84 g, 100%).

### Bis(2-ethylhexyl) 5,5′-((5-(hydroxymethyl)-1,3-phenylene)bis(oxy))dipentanoate (58)

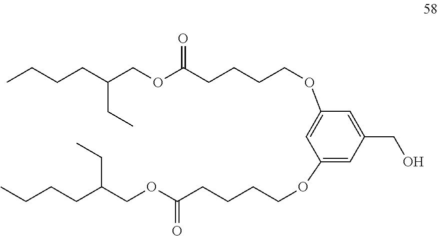 [SMILES: CCCCC(CC)COC(=O)CCCCOc1cc(CO)cc(OCCCCC(=O)OCC(CC)CCCC)c1]

To a solution of compound 57 (1.78 g, 3.08 mmol) in 50 mL THF was added NaBH₄ (0.35 g, 9.23 mmol). The reaction mixture was stirred at 23° C. for 30 min under N₂ until no more gas was generated. A solution of iodine (1.02 g, 4.00 mmol) in THF (20 mL) was added dropwise into the reaction mixture under N₂ in 20 min at 0° C. After stirring at 0° C. for 30 min, the reaction was returned to 23° C. and stirred for 1.5 h. The reaction was quenched with HCl (2 M) solution (10 mL) until no more gas was generated, and then saturated NaHCO₃aqueous solution was added until pH=7. The mixture was extracted by DCM (50 mL×3). The organic phase was dried over anhydrous MgSO₄ and filtered. The alcohol as the product (1.70 g, 98%) was obtained by concentrating the filtrate as colorless oil without further purification.

### Compound 59.

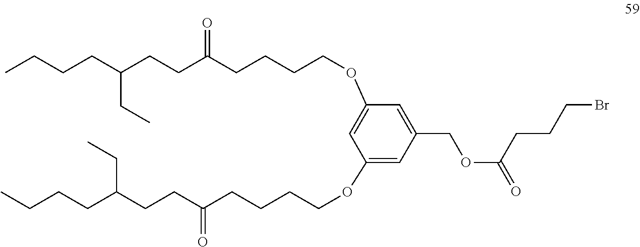 [SMILES: CCCCC(CC)CCC(=O)CCCCOc1cc(COC(=O)CCCBr)cc(OCCCCC(=O)CCC(CC)CCCC)c1]

Compound 59 was synthesized from compound 58 (1.70 g, 3.0 mmol), 4-bromobutyric acid (0.60 g, 3.6 mmol) and NEt₃ (0.36 g, 3.6 mmol) following a procedure similar to that used for the synthesis of compound 52a. Besides, DMAP (24 mg, 0.2 mmol) was added together with NEt₃ to perform the esterification. The title compound was obtained as a colorless oil (0.90 g, 42%).

### Compound 60 (IAJD95).

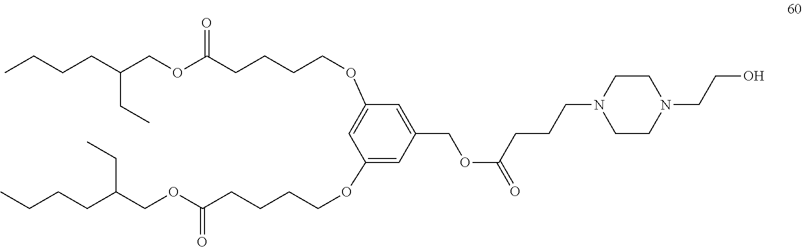 [SMILES: CCCCC(CC)COC(=O)CCCCOc1cc(COC(=O)CCCN2CCN(CCO)CC2)cc(OCCCCC(=O)OCC(CC)CCCC)c1]

Compound 60 was synthesized from compound 59 (0.45 g, 0.63 mmol, 1.0 equiv), 1-(2-hydroxyethyl)piperazine (90 mg, 0.69 mmol, 1.1 equiv) and K₂CO₃ (95 mg, 0.69 mmol, 1.1 equiv) following a procedure similar to that used for the synthesis of compound 53a. The title compound was obtained as a colorless oil (0.38 g, 82%).

Synthesis of IAJDs 96-99 and 103 was performed according to Scheme 8.

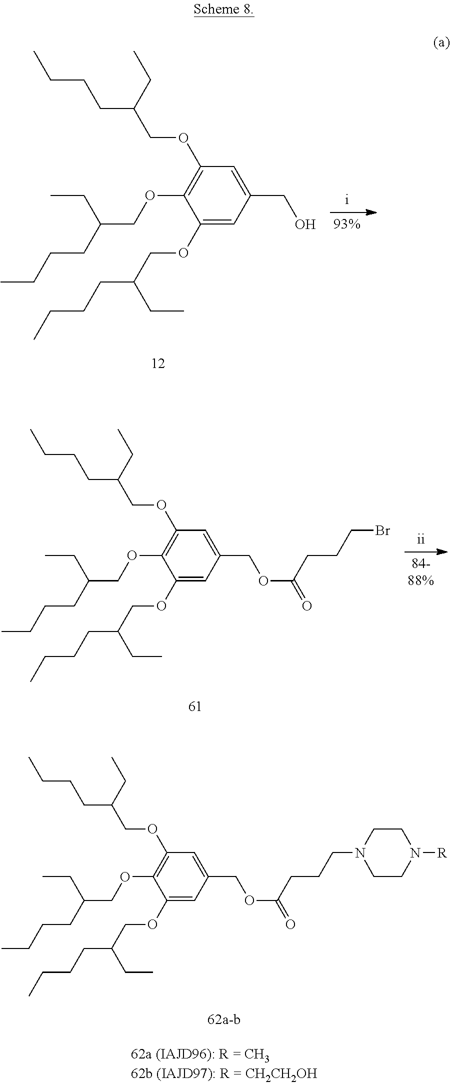

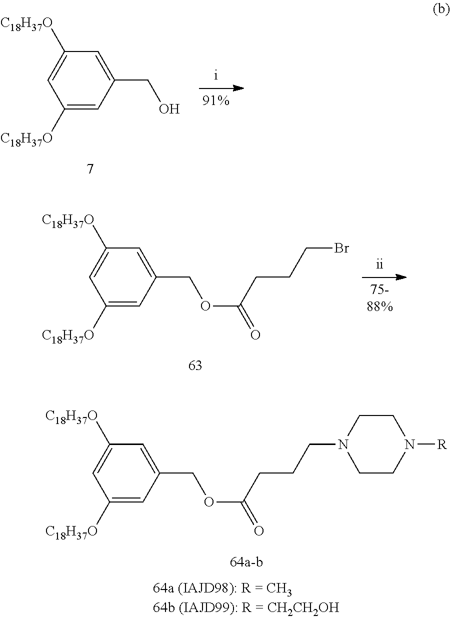

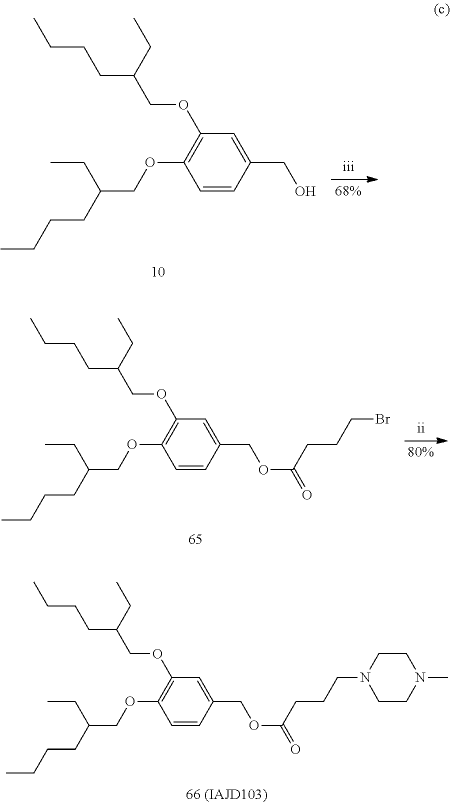 [SMILES: CCCCC(CC)COc1ccc(CO)cc1OCC(CC)CCCC ; CCCCC(CC)COc1ccc(COC(=O)CCCBr)cc1OCC(CC)CCCC ; CCCCC(CC)COc1ccc(COC(=O)CCCN2CCN(C)CC2)cc1OCC(CC)CCCC]

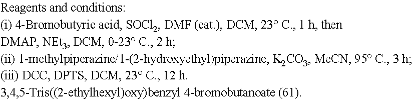

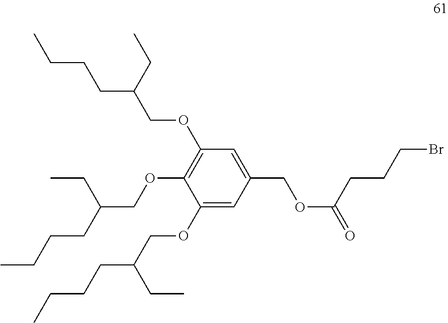 [SMILES: CCCCC(CC)COc1cc(COC(=O)CCCBr)cc(OCC(CC)CCCC)c1OCC(CC)CCCC]

4-Bromobutyric acid (0.33 g, 1.95 mmol, 1.1 equiv) was dissolved in 6 mL anhydrous DCM and 1 drop of DMF was added. SOCl₂ (0.42 g, 3.54 mmol, 2.0 equiv) was added and the mixture was stirred at 23° C. for 1 h. Afterwards, DCM and excess SOCl₂ was removed under reduced pressure to give the 4-bromobutanoyl chloride. Compound 12 (0.87 g, 1.77 mmol, 1.0 equiv) and NEt₃ (0.27 g, 2.66 mmol, 1.5 equiv) were dissolved in 6 mL anhydrous DCM. To this mixture was added the DCM solution (4 mL) of above 4-bromobutanoyl chloride dropwise at 0° C. The mixture was allowed to warm to 23° C. with stirring during a 2 h period. Afterwards, water (20 mL) was added, and the mixture was extracted by DCM (20 mL) for three times. The organic phase was collected, dried over anhydrous MgSO₄ and filtered. The filtrate was concentrated and purified by column chromatography (SiO₂) with hexane/EtOAc=20/1 as the eluent to give the title compound as a colorless oil (1.06 g, 93%).

### 3,4,5-Tris((2-ethylhexyl)oxy)benzyl 4-(4-methylpiperazin-1-yl)butanoate (62a, IAJD96)

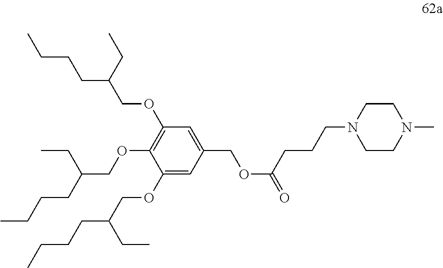 [SMILES: CCCCC(CC)COc1cc(COC(=O)CCCN2CCN(C)CC2)cc(OCC(CC)CCCC)c1OCC(CC)CCCC]

Compound 61 (0.30 g, 0.47 mmol, 1.0 equiv), 1-methylpiperazine (49 mg, 0.49 mmol, 1.05 equiv), K₂CO₃ (68 mg, 0.49 mmol, 1.05 equiv) were stirred in MeCN (20 mL). The mixture was heated at reflux (95° C.) for 3 h. The reaction mixture was cooled to 23° C. and MeCN was removed under reduced pressure. Then water (20 mL) was added, and the mixture was extracted by DCM (20 mL×3). The organic phase was collected, dried over anhydrous MgSO₄, filtered and dried to give the crude product. The crude product was purified by column chromatography (SiO₂) with DCM/MeOH=40/1 and 15/1 as the eluent. Then the obtained product was dissolved in DCM (20 mL), which was washed by NaHCO₃solution (2%, 20 mL). The aqueous phase was extracted by DCM (20 mL) for another two times. The organic phase was combined and dried over anhydrous MgSO₄. Filtration and evaporation of the solvent yielded the title compound as a light-yellow oil (0.26 g, 84%).

### 3,4,5-Tris((2-ethylhexyl)oxy)benzyl 4-(4-(2-hydroxyethyl)piperazin-1-yl)butanoate (62b, IAJD97)

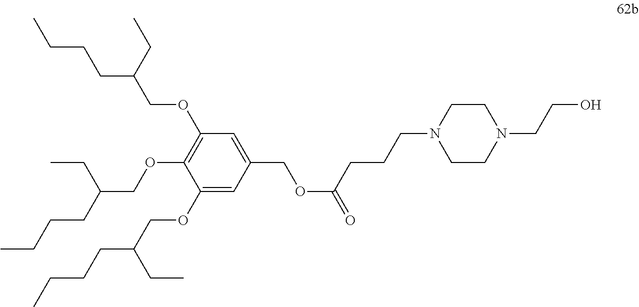 [SMILES: CCCCC(CC)COc1cc(COC(=O)CCCN2CCN(CCO)CC2)cc(OCC(CC)CCCC)c1OCC(CC)CCCC]

Compound 62b was synthesized from compound 61 (0.30 g, 0.47 mmol, 1.0 equiv), 1-(2-hydroxyethyl)piperazine (64 mg, 0.49 mmol, 1.05 equiv) and K₂CO₃ (68 mg, 0.49 mmol, 1.05 equiv) following a procedure similar to that used for the synthesis of compound 62a. The title compound was obtained as a light-yellow oil (0.28 g, 88%).

### 3,5-Bis(octadecyloxy)benzyl 4-bromobutanoate (63)

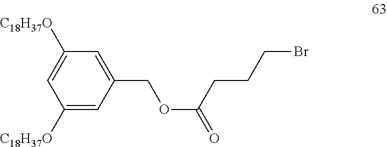

Compound 63 was synthesized from compound 7 (0.85 g, 1.32 mmol, 1.0 equiv), 4-bromobutyric acid (0.24 g, 1.45 mmol, 1.1 equiv) and NEt₃ (0.15 g, 1.45 mmol, 1.1 equiv) following a procedure similar to that used for the synthesis of compound 61. Besides, DMAP (32 mg, 0.26 mmol, 0.2 equiv) was added together with NEt₃ to perform the esterification. The title compound was obtained as a colorless oil (0.94 g, 91%).

### 3,5-Bis(octadecyloxy)benzyl 4-(4-methylpiperazin-1-yl)butanoate (64a, IAJD98)

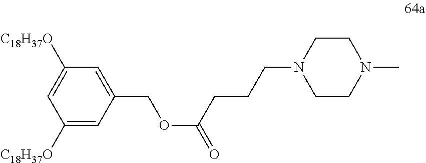

Compound 64a was synthesized from compound 63 (0.30 g, 0.38 mmol, 1.0 equiv), 1-methylpiperazine (41 mg, 0.40 mmol, 1.05 equiv) and K₂CO₃ (58 mg, 0.42 mmol, 1.1 equiv) following a procedure similar to that used for the synthesis of compound 62a. The title compound was obtained as a light-yellow oil (0.27 g, 88%).

### 3,5-Bis(octadecyloxy)benzyl 4-(4-(2-hydroxyethyl)piperazin-1-yl)butanoate (64b, IAJD99)

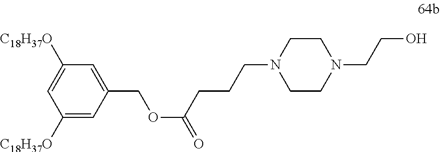

Compound 64b was synthesized from compound 63 (0.30 g, 0.38 mmol, 1.0 equiv), 1-(2-hydroxyethyl)piperazine (53 mg, 0.40 mmol, 1.05 equiv) and K₂CO₃ (58 mg, 0.42 mmol, 1.1 equiv) following a procedure similar to that used for the synthesis of compound 62a. The title compound was obtained as a light-yellow oil (0.24 g, 75%).

### 3,4-Bis((2-ethylhexyl)oxy)benzyl 4-bromobutanoate (65)

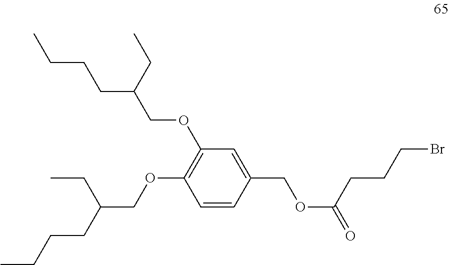 [SMILES: CCCCC(CC)COc1ccc(COC(=O)CCCBr)cc1OCC(CC)CCCC]

To a DCM (10 mL) solution of compound 10 (0.60 g, 1.65 mmol), 4-bromobutyric acid (0.28 g, 1.65 mmol) and DPTS (0.53 g, 1.81 mmol), was added DCC (0.68 g, 3.30 mmol). The mixture was allowed to stir at 23° C. for 12 h. The precipitate was filtered, and the filtrate was concentrated to dryness. The crude product was further purified by column chromatography with a mobile phase of hexane/DCM=1/1 to yield the title compound as a colorless oil (0.58 g, 68%). 3,4-Bis((2-ethylhexyl)oxy)benzyl 4-(4-methylpiperazin-1-yl)butanoate (66, IAJD103).

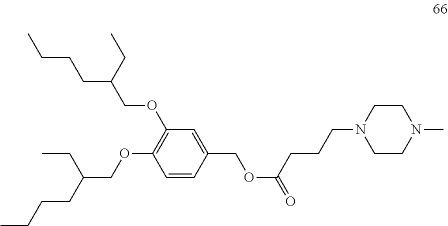 [SMILES: CCCCC(CC)COc1ccc(COC(=O)CCCN2CCN(C)CC2)cc1OCC(CC)CCCC]

Compound 66 was synthesized from compound 65 (0.54 g, 1.05 mmol, 1.0 equiv), 1-methylpiperazine (0.12 g, 1.16 mmol, 1.1 equiv) and K₂CO₃ (0.16 g, 1.16 mmol, 1.1 equiv) following a procedure similar to that used for the synthesis of compound 62a. The title compound was obtained as a colorless oil (0.45 g, 80%).

Synthesis of IAJDs 105-108 was according to Scheme 9.

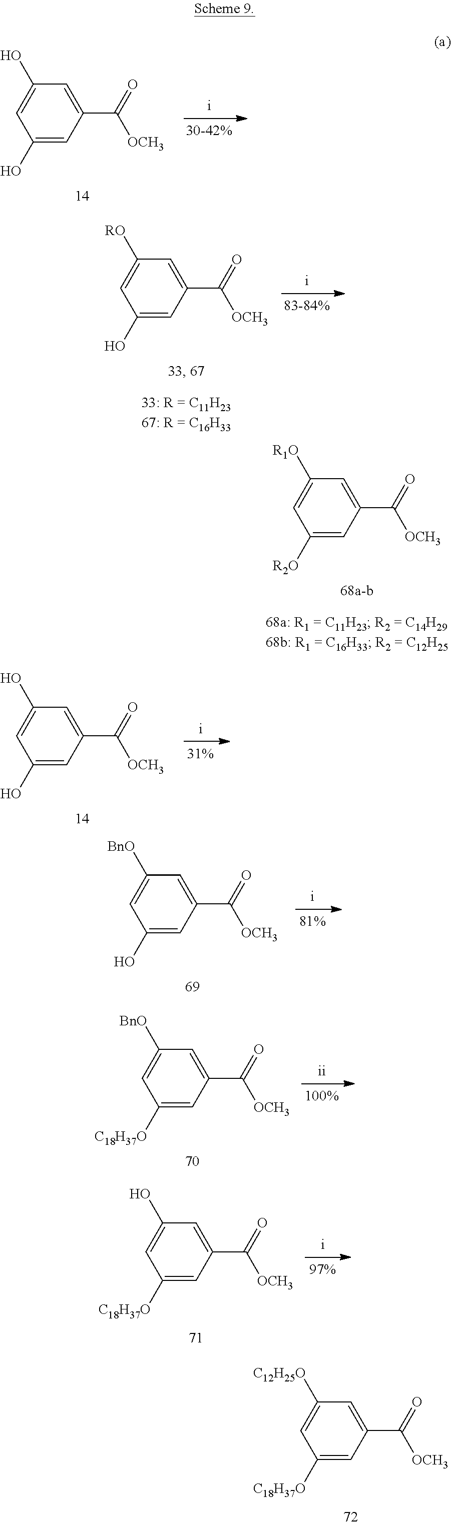

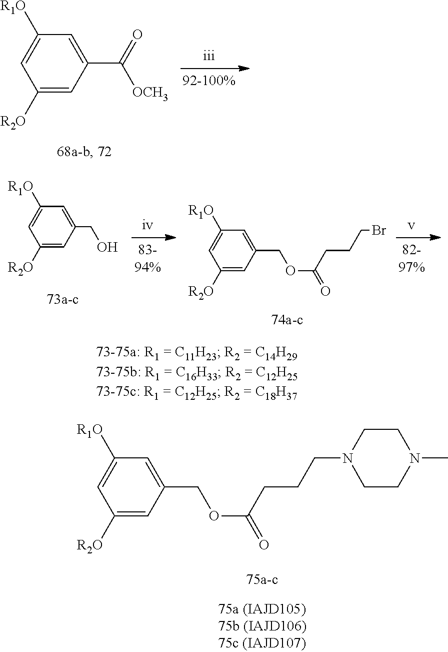

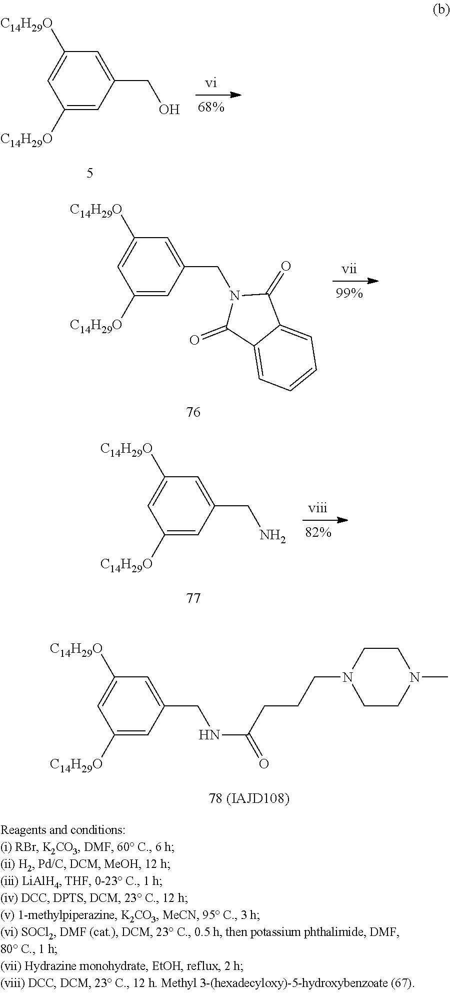

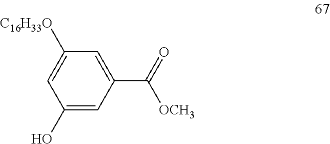

Methyl 3,5-dihydroxybenzoate (14, 2.62 g, 15.6 mmol, 1 equiv), 1-bromohexadecane (10.0 g, 32.7 mmol, 2.1 equiv), and K₂CO₃ (8.61 g, 62.4 mmol, 4 equiv) were stirred in DMF (50 mL). The mixture was stirred at 60° C. under N₂ atmosphere for 6 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with hexane/EtOAc=10/1 as the eluent to give the title compound as a white solid (1.86 g, 30%).

### Methyl 3-(tetradecyloxy)-5-(undecyloxy)benzoate (68a)

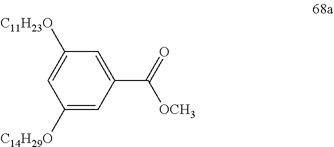

Compound 68a was synthesized from compound 33 (0.45 g, 1.39 mmol, 1.0 equiv), 1-bromotetradecane (0.41 g, 1.46 mmol, 1.05 equiv), and K₂CO₃ (0.58 g, 4.17 mmol, 3 equiv) following a procedure similar to that used for the synthesis of compound 67. The title compound was obtained as a white solid (0.60 g, 83%).

### Methyl 3-(dodecyloxy)-5-(hexadecyloxy)benzoate (68b)

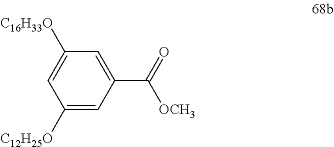

Compound 68b was synthesized from compound 67 (0.30 g, 0.76 mmol, 1.0 equiv), 1-bromododecane (0.20 g, 0.80 mmol, 1.05 equiv), and K₂CO₃ (0.21 g, 1.53 mmol, 2 equiv) following a procedure similar to that used for the synthesis of compound 67. The title compound was obtained as a white solid (0.36 g, 84%).

### Methyl 3-(benzyloxy)-5-hydroxybenzoate (69)

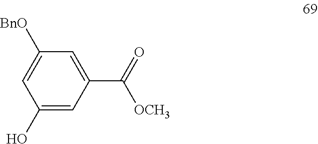 [SMILES: COC(=O)c1cc(O)cc(OCc2ccccc2)c1]

Compound 69 was synthesized from compound 14 (10.00 g, 59.5 mmol, 1.0 equiv), benzyl chloride (8.30 g, 65.4 mmol, 1.1 equiv), and K₂CO₃ (9.06 g, 65.4 mmol, 1.1 equiv) following a procedure similar to that used for the synthesis of compound 67. The title compound was obtained as a white solid (4.80 g, 31%).

### Methyl 3-(benzyloxy)-5-(octadecyloxy)benzoate (70)

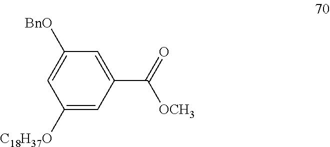

Compound 70 was synthesized from compound 69 (2.00 g, 7.74 mmol, 1.0 equiv), 1-bromooctadecane (2.70 g, 8.13 mmol, 1.05 equiv), and K₂CO₃ (2.14 g, 15.50 mmol, 2.0 equiv) following a procedure similar to that used for the synthesis of compound 67. The title compound was obtained as a white solid (3.20 g, 81%).

### Methyl 3-hydroxy-5-(octadecyloxy)benzoate (71)

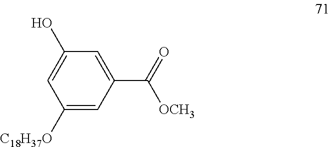

Compound 70 (3.20 g, 6.26 mmol) was dissolved in the mixture of DCM (50 mL) and methanol (10 mL). Then Pd/C (0.20 g, 6 wt %) was added and the flask was evacuated and filled with hydrogen for three times. The mixture was stirred at 23° C. for 12 h under hydrogen atmosphere. The reaction mixture was filtered through Celite and the filter cake was washed with DCM. Evaporation of the solvent yielded the title compound as a white solid (2.63 g, 100%).

### Methyl 3-(dodecyloxy)-5-(octadecyloxy)benzoate (72)

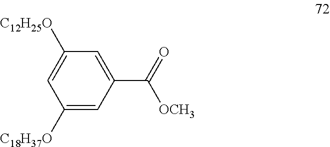

Compound 72 was synthesized from compound 71 (0.53 g, 1.26 mmol, 1.0 equiv), 1-bromododecane (0.33 g, 1.32 mmol, 1.05 equiv), and K₂CO₃ (0.35 g, 2.52 mmol, 2.0 equiv) following a procedure similar to that used for the synthesis of compound 67. The title compound was obtained as a white solid (0.72 g, 97%).

### (3-(Tetradecyloxy)-5-(undecyloxy)phenyl)methanol (73a)

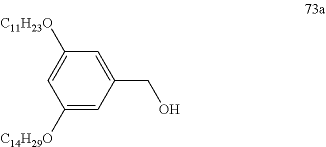

Compound 68a (0.59 g, 1.14 mmol, 1.0 equiv) was dissolved in 5 mL dry THF, which was added dropwise to a slurry of LiAlH₄ (44 mg, 1.14 mmol, 1.0 equiv) in dry THF (5 mL) at 0° C. under N₂ atmosphere. The resulted mixture was stirred at 23° C. for 1 h. The reaction was quenched by the successive addition of water (0.3 mL), 15% NaOH aqueous solution (0.3 mL) and water (1.5 mL). Then the mixture was filtered to remove white precipitates and the filtrate was dried over anhydrous MgSO₄. Filtration and evaporation of the solvent yielded the title compound as a colorless oil (0.55 g, 98%).

### (3-(Dodecyloxy)-5-(hexadecyloxy)phenyl)methanol (73b)

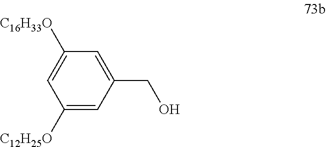

Compound 73b was synthesized from compound 68b (0.32 g, 0.57 mmol, 1.0 equiv) and LiAlH₄ (22 mg, 0.57 mmol, 1.0 equiv) following a procedure similar to that used for the synthesis of compound 73a. The title compound was obtained as a white solid (0.30 g, 100%).

### (3-(Dodecyloxy)-5-(octadecyloxy)phenyl)methanol (73c)

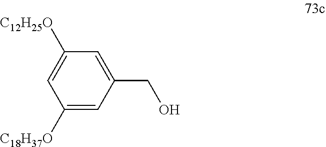

Compound 73c was synthesized from compound 72 (0.72 g, 1.22 mmol, 1.0 equiv) and LiAlH₄ (47 mg, 1.22 mmol, 1.0 equiv) following a procedure similar to that used for the synthesis of compound 73a. The title compound was obtained as a colorless oil (0.63 g, 92%).

### 3-(Tetradecyloxy)-5-(undecyloxy)benzyl 4-bromobutanoate (74a)

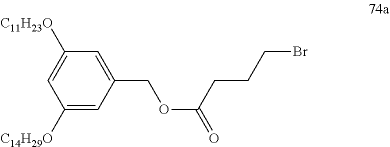

To a DCM (10 mL) solution of compound 73a (0.53 g, 1.08 mmol, 1.0 equiv), 4-bromobutyric acid (0.20 g, 1.19 mmol, 1.1 equiv) and DPTS (0.35 g, 1.19 mmol, 1.1 equiv), was added DCC (0.45 g, 2.16 mmol, 2.0 equiv). The mixture was allowed to stir at 23° C. for 12 h. The precipitate was filtered, and the filtrate was concentrated to dryness. The crude product was further purified by column chromatography with a mobile phase of hexane/DCM=1/1 to yield the title compound as a colorless oil (0.61 g, 88%).

### 3-(Dodecyloxy)-5-(hexadecyloxy)benzyl 4-bromobutanoate (74b)

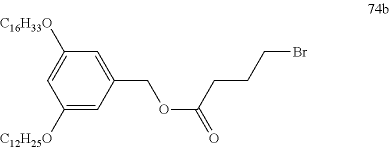

Compound 74b was synthesized from compound 73b (0.30 g, 0.56 mmol, 1.0 equiv), 4-bromobutyric acid (94 mg, 0.56 mmol, 1.0 equiv), DPTS (0.18 g, 0.62 mmol, 1.1 equiv) and DCC (0.23 g, 1.13 mmol, 2.0 equiv) following a procedure similar to that used for the synthesis of compound 74a. The title compound was obtained as a white solid (0.36 g, 94%).

### 3-(Dodecyloxy)-5-(octadecyloxy)benzyl 4-bromobutanoate (74c)

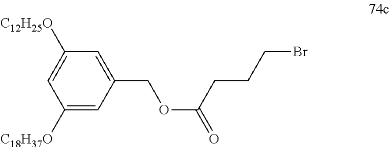

Compound 74c was synthesized from compound 73c (0.63 g, 1.12 mmol, 1.0 equiv), 4-bromobutyric acid (0.19 g, 1.12 mmol, 1.0 equiv), DPTS (0.33 g, 1.23 mmol, 1.1 equiv) and DCC (0.46 g, 2.24 mmol, 2.0 equiv) following a procedure similar to that used for the synthesis of compound 74a. The title compound was obtained as a colorless oil (0.66 g, 83%).

### 3-(Tetradecyloxy)-5-(undecyloxy)benzyl 4-(4-methylpiperazin-1-yl)butanoate (75a, IAJD105)

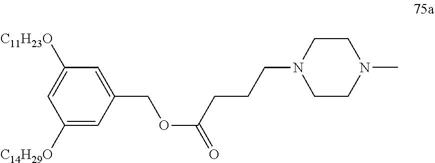

Compound 74a (0.61 g, 0.95 mmol, 1.0 equiv), 1-methylpiperazine (0.11 g, 1.10 mmol, 1.1 equiv), K₂CO₃ (0.20 g, 1.42 mmol, 1.5 equiv) were stirred in MeCN (20 mL). The mixture was heated at reflux (95° C.) for 3 h. The reaction mixture was cooled to 23° C. and MeCN was removed under reduced pressure. Then water (30 mL) was added, and the mixture was extracted by DCM (30 mL×3). The organic phase was collected, dried over anhydrous MgSO₄, filtered and dried to give the crude product. The crude product was purified by column chromatography (SiO₂) with DCM/MeOH=20/1 as the eluent. Then the obtained product was dissolved in DCM (20 mL), which was washed by NaHCO₃solution (2%, 20 mL). The aqueous phase was extracted by DCM (20 mL) for another two times. The organic phase was combined and dried over anhydrous MgSO₄.

Filtration and evaporation of the solvent yielded the title compound as a light-yellow oil (0.56 g, 90%).

### 3-(Dodecyloxy)-5-(hexadecyloxy)benzyl 4-(4-methylpiperazin-1-yl)butanoate (75b, IAJD106)

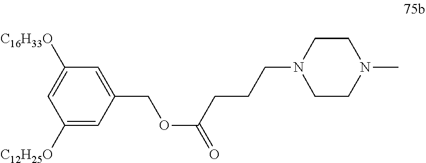

Compound 75b was synthesized from compound 74b (0.36 g, 0.53 mmol, 1.0 equiv), 1-methylpiperazine (79 mg, 0.79 mmol, 1.5 equiv) and K₂CO₃ (0.11 g, 0.79 mmol, 1.5 equiv) following a procedure similar to that used for the synthesis of compound 75a. The title compound was obtained as a light-yellow oil (0.36 g, 97%).

### 3-(Dodecyloxy)-5-(octadecyloxy)benzyl 4-(4-methylpiperazin-1-yl)butanoate (75c, IAJD107)

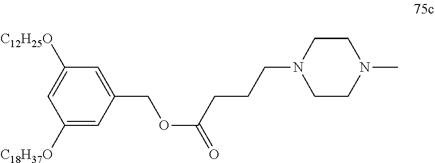

Compound 75c was synthesized from compound 74c (0.64 g, 0.90 mmol, 1.0 equiv), 1-methylpiperazine (99 mg, 0.99 mmol, 1.1 equiv) and K₂CO₃ (0.15 g, 1.10 mmol, 1.2 equiv) following a procedure similar to that used for the synthesis of compound 75a. The title compound was obtained as a light-yellow oil (0.54 g, 82%).

### 2-(3,5-Bis(tetradecyloxy)benzyl)isoindoline-1,3-dione (76)

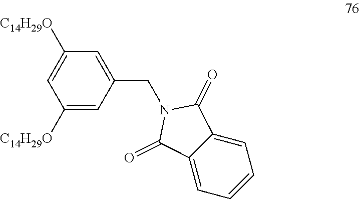

To a DCM solution (20 mL) of compound 5 (4.14 g, 7.77 mmol, 1.0 equiv) was added 2 drops of DMF, followed by the dropwise addition of SOCl₂ (1.12 g, 9.37 mmol, 1.2 equiv). The reaction mixture was stirred at 23° C. for 0.5 h. Then DCM and excess SOCl₂ was removed under reduced pressure. The obtained intermediate product was dissolved in dry DMF (40 mL) and potassium phthalimide (1.73 g, 9.32 mmol, 1.2 equiv) was added. The mixture was stirred at 80° C. under N₂ atmosphere for 1 h. The reaction mixture was cooled to 23° C. and DMF was removed under reduced pressure. Then water (30 mL) was added, and the mixture was extracted by DCM (30 mL×3). The organic phase was dried over anhydrous MgSO₄ and filtered. The filtrate was concentrated and purified by column chromatography (SiO₂) with hexane/EtOAc=20/1 as the eluent to give the title compound as a white solid (3.48 g, 68%).

### (3,5-Bis(tetradecyloxy)phenyl)methanamine (77)

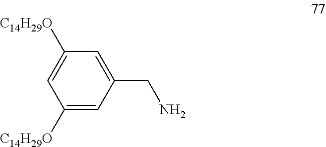

Compound 76 (1.00 g, 1.50 mmol, 1 equiv) and hydrazine monohydrate (NH₂NH₂·H₂O, 0.75 g, 15.0 mmol, 10 equiv) were dissolved in EtOH (20 mL). The mixture was heated at reflux for 2 h. The reaction mixture was cooled to 23° C. and EtOH was removed under reduced pressure. Then water (10 mL) was added, and the mixture was extracted by DCM (10 mL×3). The organic phase was dried over anhydrous MgSO₄ and filtered. Filtration and evaporation of the solvent yielded the title compound as a white solid (0.85 g, 99%).

### N-(3,5-bis(tetradecyloxy)benzyl)-4-(4-methylpiperazin-1-yl)butanamide (78, IAJD108)

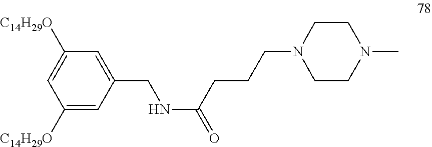

Compound 77 (0.25 g, 0.47 mmol, 1 equiv) and 4-(4-methylpiperazin-1-yl)butanoic acid hydrochloride (24b, 0.10 g, 0.47 mmol, 1 equiv) were dissolved in 6 mL dry DCM. NEt₃ (3 drops) was added. DCC (0.19 g, 0.94 mmol, 2 equiv) was added in one portion into the above mixture. The reaction was allowed to stir at 23° C. for 12 h. Afterwards, urea was removed by filtration, and washed with DCM carefully. The filtrate was concentrated and purified by column chromatography (SiO₂) with DCM/MeOH=30/1, 10/1, 8/1 and 8/1 with 0.1% TEA as the eluent. Then the product was dissolved in DCM (20 mL), which was washed by NaHCO₃solution (2%, 20 mL). The aqueous phase was extracted by DCM (20 mL) for another two times. The organic phase was combined and dried over anhydrous MgSO₄. Filtration and evaporation of the solvent yielded the title compound as a white solid (0.27 g, 82%).

Dimensions of DNPs Containing Luc-mRNA Obtained by Co-assembly of IAJDs with Luc-mRNA by Injection in Acetate Buffer (10 mM, pH 4.0) and Luminescence Expression in HEK293T Cells (125 ng/well, n=3) are shown in Table 12.

TABLE 12

| | Buffer | C_{IAJD} | C_{Luc-mRNA} | Size | | | Standard |
|---|---|---|---|---|---|---|---|
| IAJD | (10 mM) | (mg/mL) | (mg/mL) | (d, nm) | PDI | Luminescence | deviation |
| 64 | Acetate | 4 | 0.10 | 135 | 0.271 | 125 | 7 |
| | (pH 4.0) | | | | | | |
| 65 | Acetate | 4 | 0.10 | 241 | 0.249 | 18 | 2 |
| | (pH 4.0) | | | | | | |
| 66 | Acetate | 4 | 0.10 | 165 | 0.320 | 234 | 43 |
| | (pH 4.0) | | | | | | |
| 70 | Acetate | 4 | 0.10 | 205 | 0.438 | 455,361 | 66,604 |
| | (pH 4.0) | | | | | | |
| 71 | Acetate | 4 | 0.10 | 237 | 0.451 | 473,548 | 123,589 |
| | (pH 4.0) | | | | | | |
| 74 | Acetate | 4 | 0.10 | 180 | 0.317 | 3,926 | 1,559 |
| | (pH 4.0) | | | | | | |
| 75 | Acetate | 4 | 0.10 | 235 | 0.328 | 68,572 | 6,794 |
| | (pH 4.0) | | | | | | |
| 76 | Acetate | 4 | 0.10 | 78 | 0.287 | 17 | 3 |
| | (pH 4.0) | | | | | | |
| 77 | Acetate | 4 | 0.10 | 110 | 0.267 | 129 | 6 |
| | (pH 4.0) | | | | | | |
| 78 | Acetate | 4 | 0.10 | 629 | 1.000 | 8,688 | 1,914 |
| | (pH 4.0) | | | | | | |
| 79 | Acetate | 4 | 0.10 | 352 | 0.763 | 27,615 | 6,704 |
| | (pH 4.0) | | | | | | |
| 81 | Acetate | 4 | 0.10 | 111 | 0.287 | 206,161 | 21,200 |
| | (pH 4.0) | | | | | | |
| 82 | Acetate | 4 | 0.10 | 162 | 0.144 | 17 | 2 |
| | (pH 4.0) | | | | | | |
| 83 | Acetate | 4 | 0.10 | 143 | 0.430 | 68,691 | 2,498 |
| | (pH 4.0) | | | | | | |
| 84 | Acetate | 4 | 0.10 | 171 | 0.303 | 205 | 152 |
| | (pH 4.0) | | | | | | |
| 85 | Acetate | 4 | 0.10 | 102 | 0.250 | 47,369 | 18,262 |
| | (pH 4.0) | | | | | | |
| 86 | Acetate | 4 | 0.10 | 91 | 0.234 | 47,066 | 21,163 |
| | (pH 4.0) | | | | | | |
| 87 | Acetate | 4 | 0.10 | 146 | 0.486 | 33 | 7 |
| | (pH 4.0) | | | | | | |
| 88 | Acetate | 4 | 0.10 | 100 | 0.342 | 17 | 2 |
| | (pH 4.0) | | | | | | |
| 89 | Acetate | 4 | 0.10 | 149 | 0.467 | 55 | 12 |
| | (pH 4.0) | | | | | | |
| 91 | Acetate | 4 | 0.10 | 188 | 0.498 | 65,255 | 5,573 |
| | (pH 4.0) | | | | | | |
| 95 | Acetate | 4 | 0.10 | 105 | 0.352 | 153,564 | 25,782 |
| | (pH 4.0) | | | | | | |
| 96 | Acetate | 4 | 0.10 | 139 | 0.402 | 22,799 | 4,244 |
| | (pH 4.0) | | | | | | |
| 97 | Acetate | 4 | 0.10 | 204 | 0.482 | 342,016 | 70,616 |
| | (pH 4.0) | | | | | | |
| 98 | Acetate | 4 | 0.10 | 187 | 0.221 | 22,209 | 10,213 |
| | (pH 4.0) | | | | | | |
| 99 | Acetate | 4 | 0.10 | 277 | 0.571 | 87,433 | 7,107 |
| | (pH 4.0) | | | | | | |
| 103 | Acetate | 4 | 0.10 | 88 | 0.275 | 19 | 8 |
| | (pH 4.0) | | | | | | |
| 105 | Acetate | 4 | 0.10 | 94 | 0.294 | 85 | 21 |
| | (pH 4.0) | | | | | | |
| 106 | Acetate | 4 | 0.10 | 105 | 0.275 | 309 | 116 |
| | (pH 4.0) | | | | | | |
| 107 | Acetate | 4 | 0.10 | 146 | 0.279 | 93,745 | 12,116 |
| | (pH 4.0) | | | | | | |
| 108 | Acetate | 4 | 0.10 | 214 | 0.334 | 136,424 | 3,167 |
| | (pH 4.0) | | | | | | |
| Negative | | | | | | 81 | 5 |
| Control | | | | | | | |
| (untreated | | | | | | | |
| cells) | | | | | | | |
| MC3-Luc- | | | | | | 1,320,533 | 105,967 |
| mRNA | | | | | | | |

Quantification of luminescence expression in vivo is shown in Table 13.

TABLE 13

| | | | pH | | | | | Total | |
|---|---|---|---|---|---|---|---|---|---|
| | C_{IAJD} | C_{Luc-mRNA} | Acetate | | Size | | T | flux | Organs with |
| IAJD | (mg/mL) | (mg/mL) | buffer | Sample | (d, nm) | PDI | (h)^{a} | (p/s) | luminescence |
| 64 | 4 | 0.10 | 4.0 | 4.53 | 135 | 0.271 | 4 | 0 | — |
| 65 | 4 | 0.10 | 4.0 | 4.22 | 241 | 0.249 | 4 | 0 | — |
| 66 | 4 | 0.10 | 4.0 | 4.50 | 165 | 0.320 | 4 | 2.58 × | Lung, liver, |
| | | | | | | | | 10⁷ | spleen |
| 70 | 4 | 0.10 | 4.0 | 4.50 | 205 | 0.438 | 5 | 6.62 × | Lung, spleen |
| | | | | | | | | 10⁶ | |
| 71 | 4 | 0.10 | 4.0 | 4.39 | 237 | 0.451 | 5 | 3.79 × | Lung, liver, |
| | | | | | | | | 10⁷ | spleen |
| 74 | 4 | 0.10 | 4.0 | 4.46 | 180 | 0.317 | 5 | 1.71 × | Lung, liver, |
| | | | | | | | | 10⁷ | spleen |
| 75 | 4 | 0.10 | 4.0 | 4.62 | 235 | 0.328 | 5 | 3.37 × | Lung, liver, |
| | | | | | | | | 10⁷ | spleen |
| 76 | 4 | 0.10 | 4.0 | 4.47 | 78 | 0.287 | 5 | 7.81 × | Lung, liver, |
| | | | | | | | | 10⁷ | spleen |
| 77 | 4 | 0.10 | 4.0 | 4.50 | 110 | 0.267 | 5 | 2.44 × | Lung, liver, |
| | | | | | | | | 10⁶ | spleen |
| 78 | 4 | 0.10 | 4.0 | 4.46 | 629 | 1.000 | 5 | 1.12 × | Lung, spleen |
| | | | | | | | | 10⁵ | |
| 79 | 4 | 0.10 | 4.0 | 4.43 | 352 | 0.763 | 6 | 1.34 × | Lung, liver, |
| | | | | | | | | 10⁷ | spleen |
| 81 | 4 | 0.10 | 4.0 | 4.38 | 111 | 0.287 | 6 | 2.32 × | Lung, liver, |
| | | | | | | | | 10⁷ | spleen |
| 82 | 4 | 0.10 | 4.0 | 4.26 | 162 | 0.144 | 5 | 1.62 × | spleen |
| | | | | | | | | 10⁶ | |
| 83 | 4 | 0.10 | 4.0 | 4.31 | 143 | 0.430 | 5 | 7.11 × | Lung, liver, |
| | | | | | | | | 10⁷ | spleen |
| 84 | 4 | 0.10 | 4.0 | 4.44 | 171 | 0.303 | 5 | 6.89 × | Lung, liver, |
| | | | | | | | | 10⁵ | spleen |
| 85 | 4 | 0.10 | 4.0 | 4.44 | 102 | 0.250 | 5 | 2.87 × | Lung, liver, |
| | | | | | | | | 10⁶ | spleen |
| 86 | 4 | 0.10 | 4.0 | 4.39 | 91 | 0.234 | 5 | 4.73 × | Lung, liver, |
| | | | | | | | | 10⁷ | spleen |
| 87 | 4 | 0.10 | 4.0 | 4.47 | 146 | 0.486 | 6 | 3.24 × | Lung, liver, |
| | | | | | | | | 10⁷ | spleen |
| 88 | 4 | 0.10 | 4.0 | 4.55 | 100 | 0.342 | | 2.34 × | Lung, liver, |
| | | | | | | | 6 | 10⁶ | spleen |
| 89 | 4 | 0.10 | 4.0 | 4.45 | 149 | 0.467 | | 5.92 × | Lung, liver, |
| | | | | | | | 6 | 10⁷ | spleen |
| 91 | 4 | 0.10 | 4.0 | 4.44 | 188 | 0.498 | | 9.94 × | Lung, liver, |
| | | | | | | | 6 | 10⁶ | spleen |
| 95 | 4 | 0.10 | 4.0 | 4.38 | 105 | 0.352 | | 6.61 × | Lung, liver, |
| | | | | | | | 6 | 10⁷ | spleen |
| 96 | 4 | 0.10 | 4.0 | 4.52 | 139 | 0.402 | | 4.79 × | Lung, liver, |
| | | | | | | | 5 | 10⁷ | spleen |
| 97 | 4 | 0.10 | 4.0 | 4.42 | 204 | 0.482 | | 5.89 × | Lung, liver, |
| | | | | | | | 5 | 10⁷ | spleen |
| 98 | 4 | 0.10 | 4.0 | 4.35 | 187 | 0.221 | | 2.06 × | Lung, liver, |
| | | | | | | | 6 | 10⁶ | spleen |
| 99 | 4 | 0.10 | 4.0 | 4.32 | 277 | 0.571 | | 4.49 × | Lung, liver, |
| | | | | | | | 6 | 10⁶ | spleen |
| 103 | 4 | 0.10 | 4.0 | 4.58 | 88 | 0.275 | | 1.70 × | Lung, spleen |
| | | | | | | | 6 | 10⁶ | |
| 105 | 4 | 0.10 | 4.0 | 4.42 | 94 | 0.294 | | 4.74 × | Lung, liver, |
| | | | | | | | 6 | 10⁷ | spleen |
| 106 | 4 | 0.10 | 4.0 | 4.41 | 105 | 0.275 | | 3.48 × | Lung, liver, |
| | | | | | | | 6 | 10⁷ | spleen |
| 107 | 4 | 0.10 | 4.0 | 4.42 | 146 | 0.279 | | 2.19 × | Lung, liver, |
| | | | | | | | 6 | 10⁷ | spleen |
| 108 | 4 | 0.10 | 4.0 | 4.54 | 214 | 0.334 | | 9.66 × | Lung, spleen |
| | | | | | | | 6 | 10⁶ | |

^{a}Time from Luc-mRNA-IAJD injection to the imaging of mice on the IVIS imaging system.

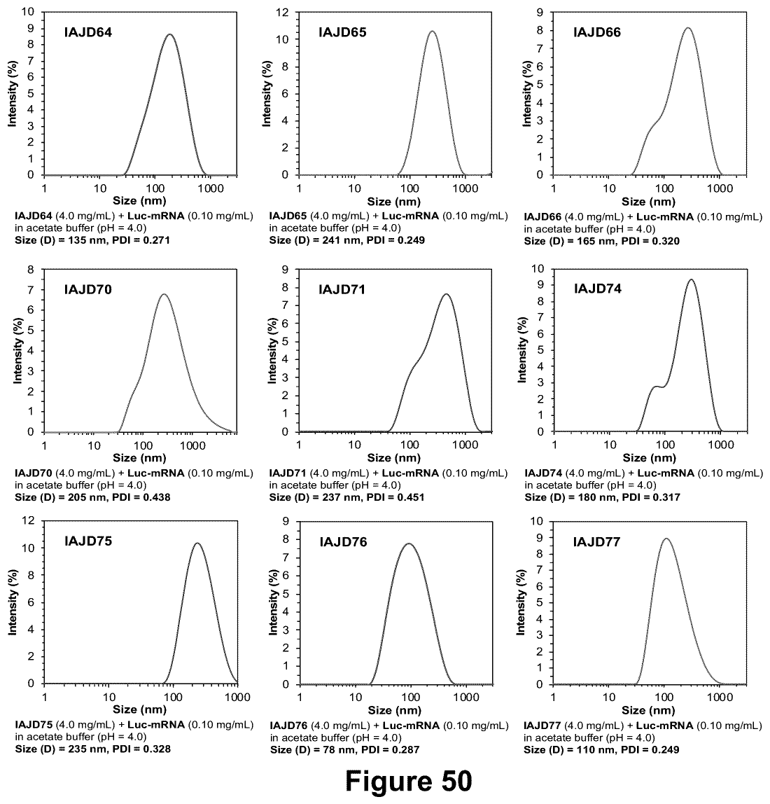

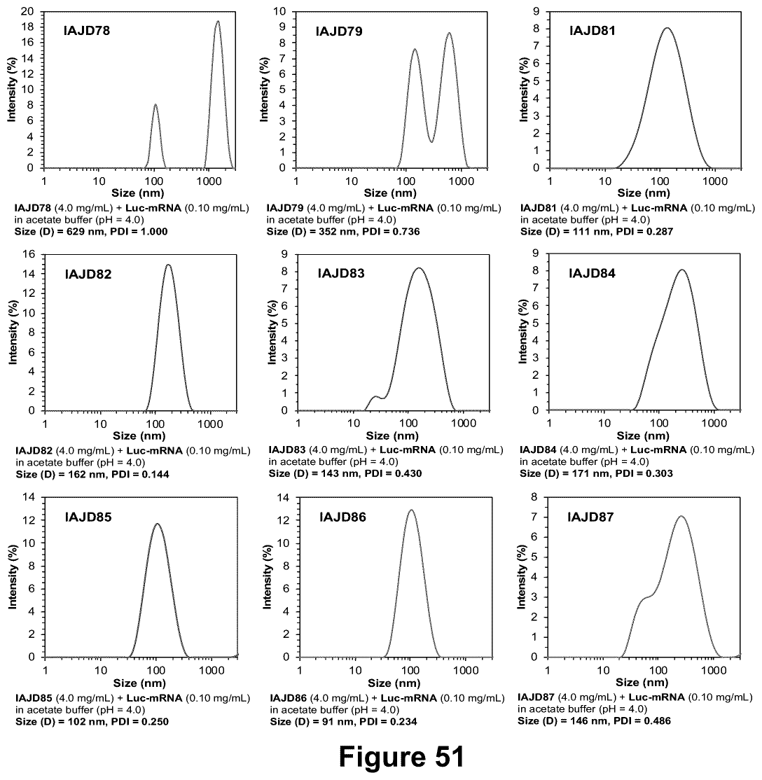

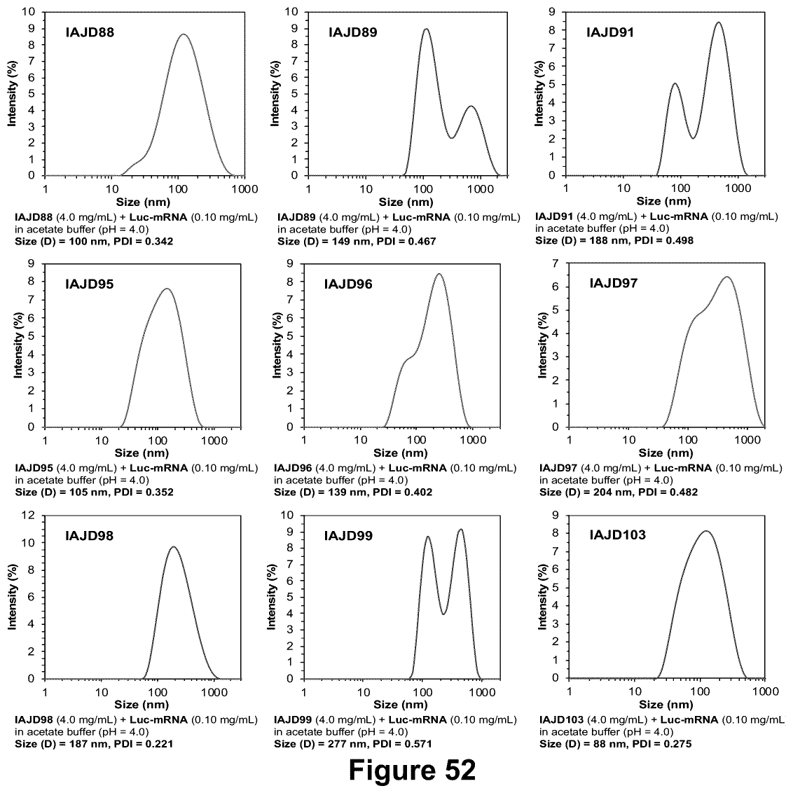

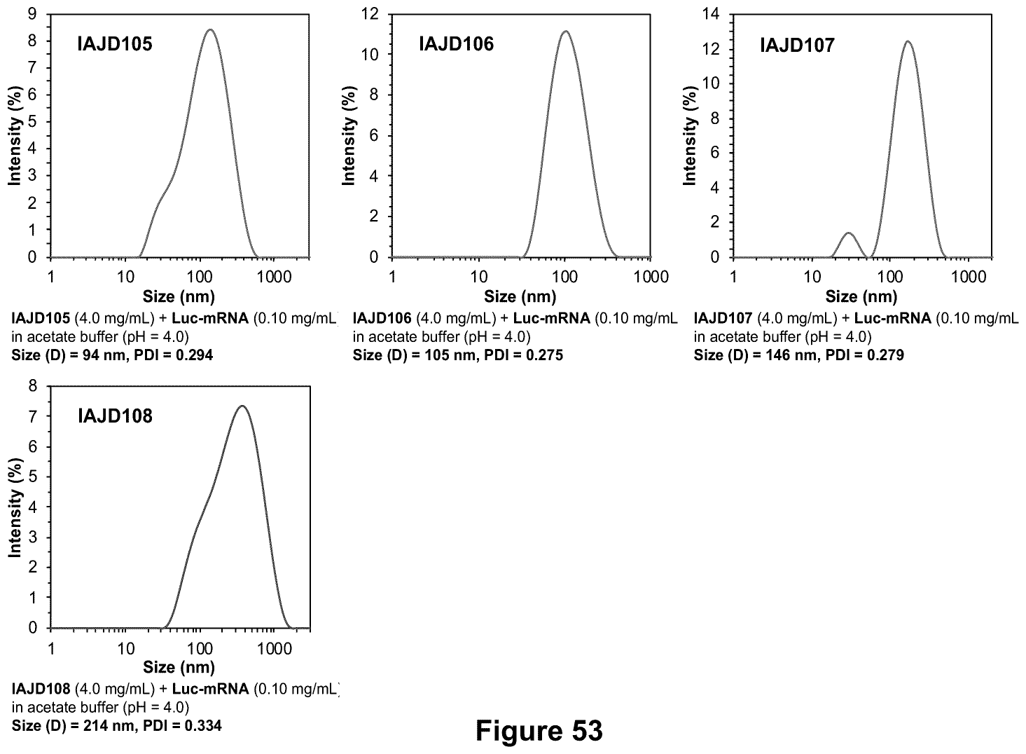

DLS data of DNPs assembled from IAJDs and Luc-mRNA is shown in FIG. 50 through FIG. 53. Stability of DNPs by determined by DLS^{a} at 5° C. after different periods of time is shown in Table 14.

TABLE 14
Stability of DNPs by determined by DLS^{a} at 5° C. after different periods of time.

| | | | pH | | | | | Total | |
|---|---|---|---|---|---|---|---|---|---|
| | C_{IAJD} | C_{Luc-mRNA} | Acetate | | Size | | T | flux | Organs with |
| IAJD | (mg/mL) | (mg/mL) | buffer | Sample | (d, nm) | PDI | (h)^{a} | (p/s) | luminescence^{b} |
| 64 | 4 | 0.10 | 4.0 | 4.53 | 135 | 0.271 | 4 | 0 | — |
| 65 | 4 | 0.10 | 4.0 | 4.22 | 241 | 0.249 | 4 | 0 | — |
| 66 | 4 | 0.10 | 4.0 | 4.50 | 165 | 0.320 | 4 | 2.58 × | Lung (weak), |
| | | | | | | | | 10⁷ | liver (strong), |
| | | | | | | | | | spleen (strong) |
| 70 | 4 | 0.10 | 4.0 | 4.50 | 205 | 0.438 | 5 | 6.62 × | Lung (strong), |
| | | | | | | | | 10⁶ | spleen (weak) |
| 71 | 4 | 0.10 | 4.0 | 4.39 | 237 | 0.451 | 5 | 3.79 × | Lung (weak), |
| | | | | | | | | 10⁷ | liver (weak), |
| | | | | | | | | | spleen (strong) |
| 74 | 4 | 0.10 | 4.0 | 4.46 | 180 | 0.317 | 5 | 1.71 × | Lung (weak), |
| | | | | | | | | 10⁷ | liver (weak), |
| | | | | | | | | | spleen (strong) |
| 75 | 4 | 0.10 | 4.0 | 4.62 | 235 | 0.328 | 5 | 3.37 × | Lung (weak), |
| | | | | | | | | 10⁷ | liver (weak), |
| | | | | | | | | | spleen (strong) |
| 76 | 4 | 0.10 | 4.0 | 4.47 | 78 | 0.287 | 5 | 7.81 × | Lung (weak), |
| | | | | | | | | 10⁷ | liver (weak), |
| | | | | | | | | | spleen (strong) |
| 77 | 4 | 0.10 | 4.0 | 4.50 | 110 | 0.267 | 5 | 2.44 × | Lung (weak), |
| | | | | | | | | 10⁶ | liver (weak), |
| | | | | | | | | | spleen (strong) |
| 78 | 4 | 0.10 | 4.0 | 4.46 | 629 | 1.000 | 5 | 1.12 × | Lung (strong), |
| | | | | | | | | 10⁵ | spleen (weak) |
| 79 | 4 | 0.10 | 4.0 | 4.43 | 352 | 0.763 | 6 | 1.34 × | Lung (weak), |
| | | | | | | | | 10⁷ | liver (weak), |
| | | | | | | | | | spleen (strong) |
| 81 | 4 | 0.10 | 4.0 | 4.38 | 111 | 0.287 | 6 | 2.32 × | Lung (weak), |
| | | | | | | | | 10⁷ | liver (strong), |
| | | | | | | | | | spleen (weak) |
| 82 | 4 | 0.10 | 4.0 | 4.26 | 162 | 0.144 | 5 | 1.62 × | Spleen (strong) |
| | | | | | | | | 10⁶ | |
| 83 | 4 | 0.10 | 4.0 | 4.31 | 143 | 0.430 | 5 | 7.11 × | Lung (weak), |
| | | | | | | | | 10⁷ | liver (weak), |
| | | | | | | | | | spleen (strong) |
| 84 | 4 | 0.10 | 4.0 | 4.44 | 171 | 0.303 | 5 | 6.89 × | Lung (weak), |
| | | | | | | | | 10⁵ | liver (weak), |
| | | | | | | | | | spleen (strong) |
| 85 | 4 | 0.10 | 4.0 | 4.44 | 102 | 0.250 | 5 | 2.87 × | Lung (weak), |
| | | | | | | | | 10⁶ | liver (weak), |
| | | | | | | | | | spleen (strong) |
| 86 | 4 | 0.10 | 4.0 | 4.39 | 91 | 0.234 | 5 | 4.73 × | Lung (weak), |
| | | | | | | | | 10⁷ | liver (strong), |
| | | | | | | | | | spleen (weak) |
| 87 | 4 | 0.10 | 4.0 | 4.47 | 146 | 0.486 | 6 | 3.24 × | Lung (weak), |
| | | | | | | | | 10⁷ | liver (weak), |
| | | | | | | | | | spleen (strong) |
| 88 | 4 | 0.10 | 4.0 | 4.55 | 100 | 0.342 | 6 | 2.34 × | Lung (weak), |
| | | | | | | | | 10⁶ | liver (weak), |
| | | | | | | | | | spleen (strong) |
| 89 | 4 | 0.10 | 4.0 | 4.45 | 149 | 0.467 | 6 | 5.92 × | Lung (weak), |
| | | | | | | | | 10⁷ | liver (weak), |
| | | | | | | | | | spleen (strong) |
| 91 | 4 | 0.10 | 4.0 | 4.44 | 188 | 0.498 | 6 | 9.94 × | Lung (weak), |
| | | | | | | | | 10⁶ | liver (weak), |
| | | | | | | | | | spleen (strong) |
| 95 | 4 | 0.10 | 4.0 | 4.38 | 105 | 0.352 | 6 | 6.61 × | Lung (weak), |
| | | | | | | | | 10⁷ | liver (weak), |
| | | | | | | | | | spleen (strong) |
| 96 | 4 | 0.10 | 4.0 | 4.52 | 139 | 0.402 | 5 | 4.79 × | Lung (weak), |
| | | | | | | | | 10⁷ | liver (weak), |
| | | | | | | | | | spleen (strong) |
| 97 | 4 | 0.10 | 4.0 | 4.42 | 204 | 0.482 | 5 | 5.89 × | Lung (weak), |
| | | | | | | | | 10⁷ | liver (weak), |
| | | | | | | | | | spleen (strong) |
| 98 | 4 | 0.10 | 4.0 | 4.35 | 187 | 0.221 | 6 | 2.06 × | Lung (weak), |
| | | | | | | | | 10⁶ | liver (weak), |
| | | | | | | | | | spleen (strong) |
| 99 | 4 | 0.10 | 4.0 | 4.32 | 277 | 0.571 | 6 | 4.49 × | Lung (weak), |
| | | | | | | | | 10⁶ | liver (weak), |
| | | | | | | | | | spleen (strong) |
| 103 | 4 | 0.10 | 4.0 | 4.58 | 88 | 0.275 | 6 | 1.70 × | Lung (strong), |
| | | | | | | | | 10⁶ | spleen (weak) |
| 105 | 4 | 0.10 | 4.0 | 4.42 | 94 | 0.294 | 6 | 4.74 × | Lung (weak), |
| | | | | | | | | 10⁷ | liver (strong), |
| | | | | | | | | | spleen (weak) |
| 106 | 4 | 0.10 | 4.0 | 4.41 | 105 | 0.275 | 6 | 3.48 × | Lung (weak), |
| | | | | | | | | 10⁷ | liver (weak), |
| | | | | | | | | | spleen (strong) |
| 107 | 4 | 0.10 | 4.0 | 4.42 | 146 | 0.279 | 6 | 2.19 × | Lung (weak), |
| | | | | | | | | 10⁷ | liver (weak), |
| | | | | | | | | | spleen (strong) |
| 108 | 4 | 0.10 | 4.0 | 4.54 | 214 | 0.334 | 6 | 9.66 × | Lung (strong), |
| | | | | | | | | 10⁶ | spleen (weak) |

^{a}All the samples are prepared in acetate buffer.
^{b}U: Unimodal size distribution of DLS curve; B: Bimodal size distribution of DLS curve; M: Multimodal size distribution of DLS curve.
c. Ranking of stability: Excellent (in green color) - the original sizes and distribution remained almost the same after storage; Good - the original sizes and distribution changed slightly (size difference was less than 100 nm) after storage or the original size remained almost the same but with multimodal distributions after storage; Not stable - the original sizes and distribution changed obviously (size difference was more than 100 nm and distribution type of DLS curve changed) after storage.

pKₐ Measurements of individual IAJD molecules is shown in Table 15.

TABLE 15

| IAJD No. | pKₐ |
|---|---|
| IAJD 64 | 7.06 |
| IAJD 65 | 7.40 |
| IAJD 66 | 6.37 |
| IAJD 70 | 6.44 |
| IAJD 71 | 6.38 |
| IAJD 74 | 6.32 |
| IAJD 75 | 6.22 |
| IAJD 76 | 6.28 |
| IAJD 77 | 6.40 |
| IAJD 78 | 6.46 |
| IAJD 79 | 6.25 |
| IAJD 81 | 6.19 |
| IAJD 82 | 6.18 |
| IAJD 83 | 6.30 |
| IAJD 84 | 6.19 |
| IAJD 85 | 6.21 |
| IAJD 86 | 6.24 |
| IAJD 87 | 6.49 |
| IAJD 88 | 6.28 |
| IAJD 89 | 6.45 |
| IAJD 91 | 6.48 |
| IAJD 95 | 6.33 |
| IAJD 96 | 6.32 |
| IAJD 97 | 6.46 |
| IAJD 98 | 6.25 |
| IAJD 99 | 6.31 |
| IAJD 103 | 6.23 |
| IAJD 105 | 6.50 |
| IAJD 106 | 6.38 |
| IAJD 107 | 6.42 |
| IAJD 108 | 6.59 |

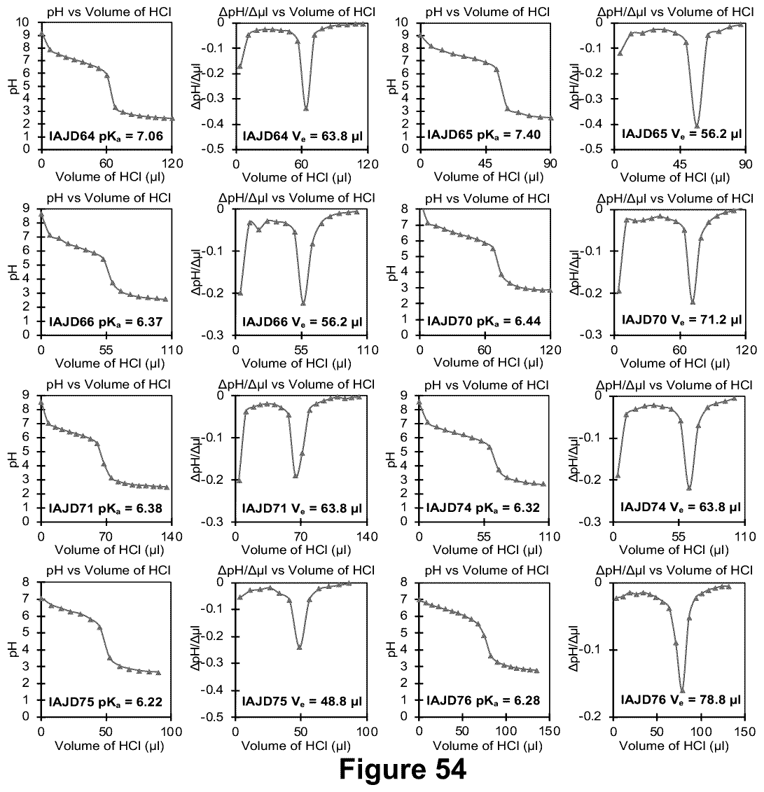

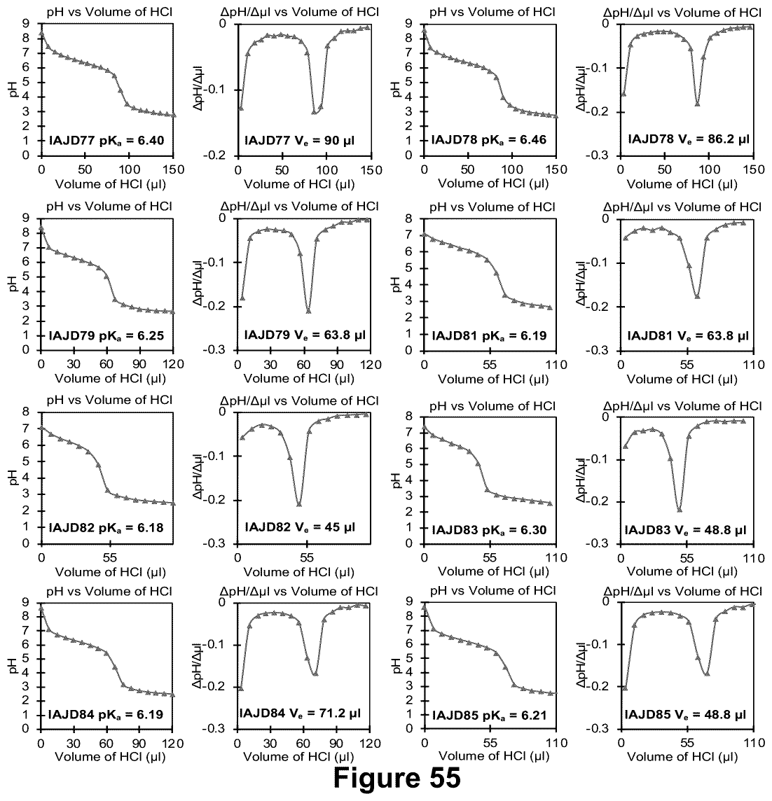

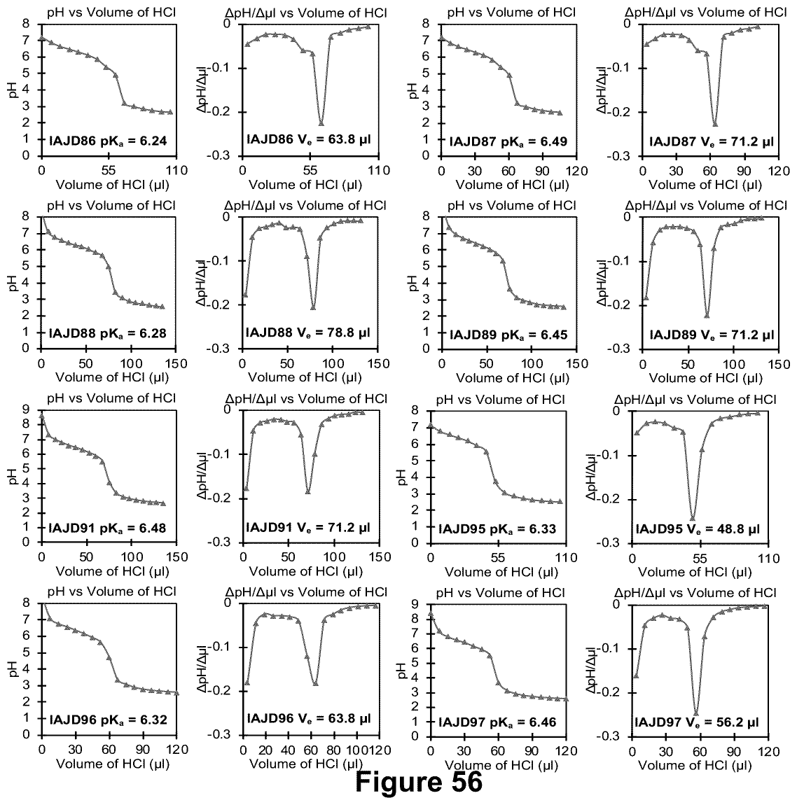

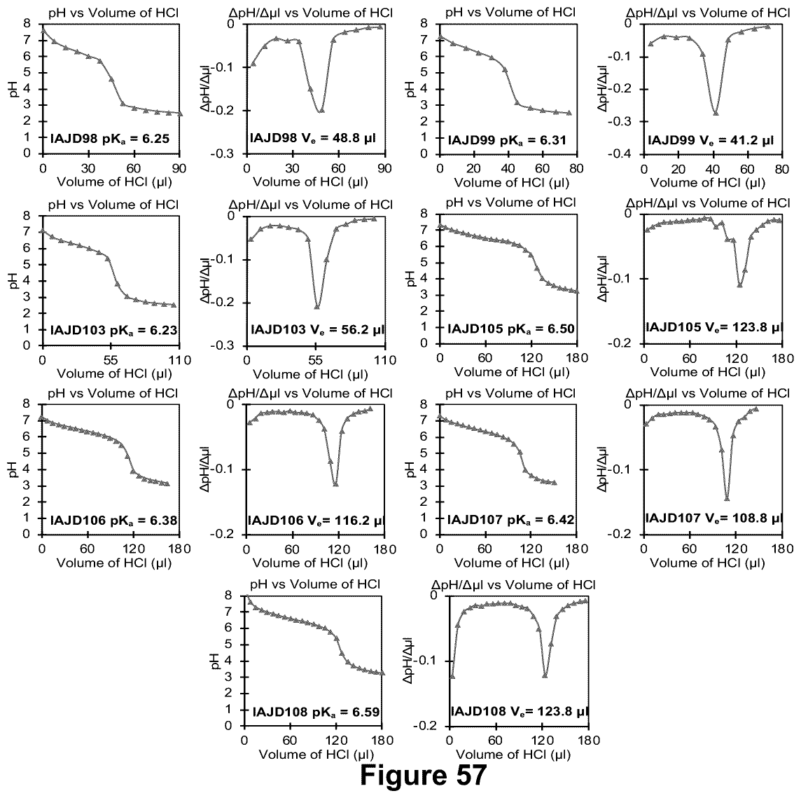

Titration curves showing changes in solution pH in response to addition of a strong acid for IAJD molecules is shown in FIG. 54 through FIG. 57.

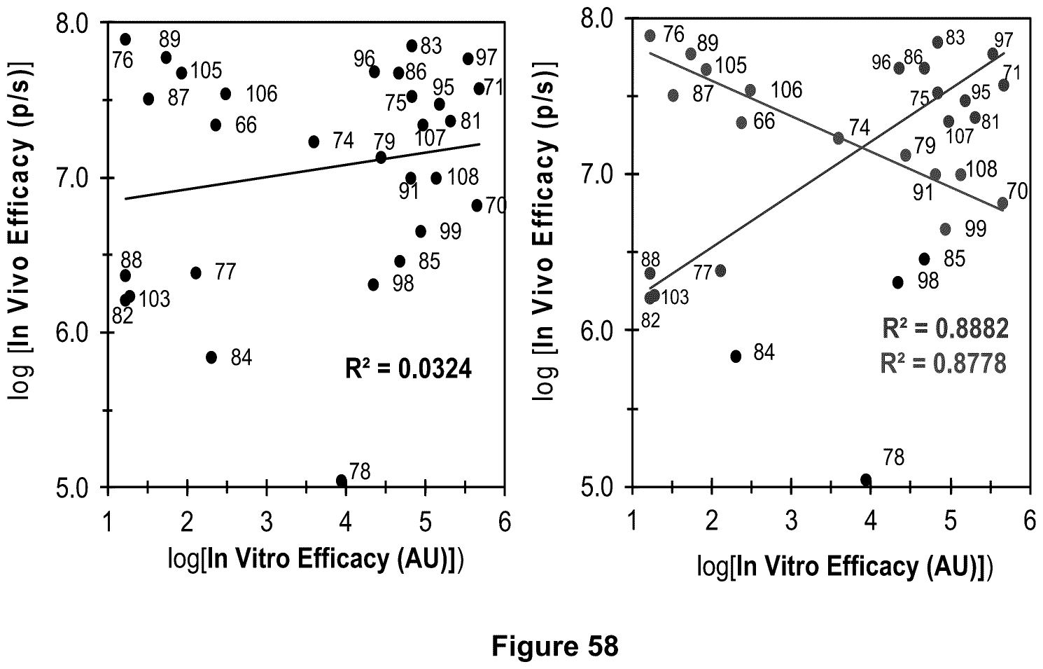

A comparison of in vitro vs in vivo efficacy is shown in FIG. 58. No correlations were found between in vitro and in vivo activities when all data points were considered; positive and negative correlations between in vitro and in vivo activities were found for two different groups of bias selective data points shown by the lines in FIG. 58 (right) (the selection of data points has no scientific base).

### Example 3: The Unexpected Importance of the Primary Structure of the Hydrophobic Part of One-Component IAJDs in Targeted mRNA Delivery Activity

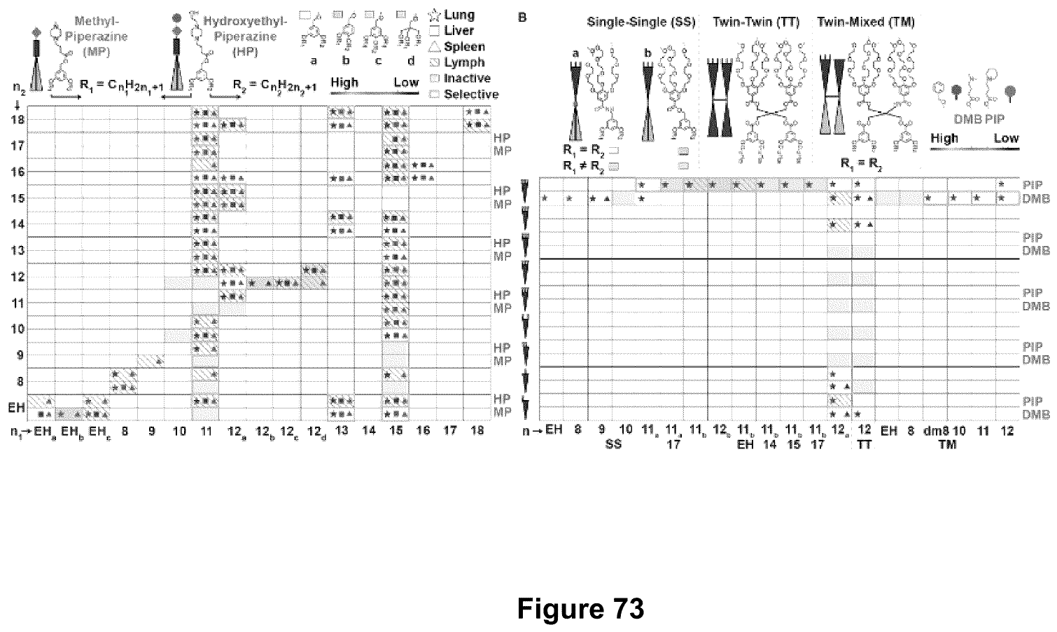

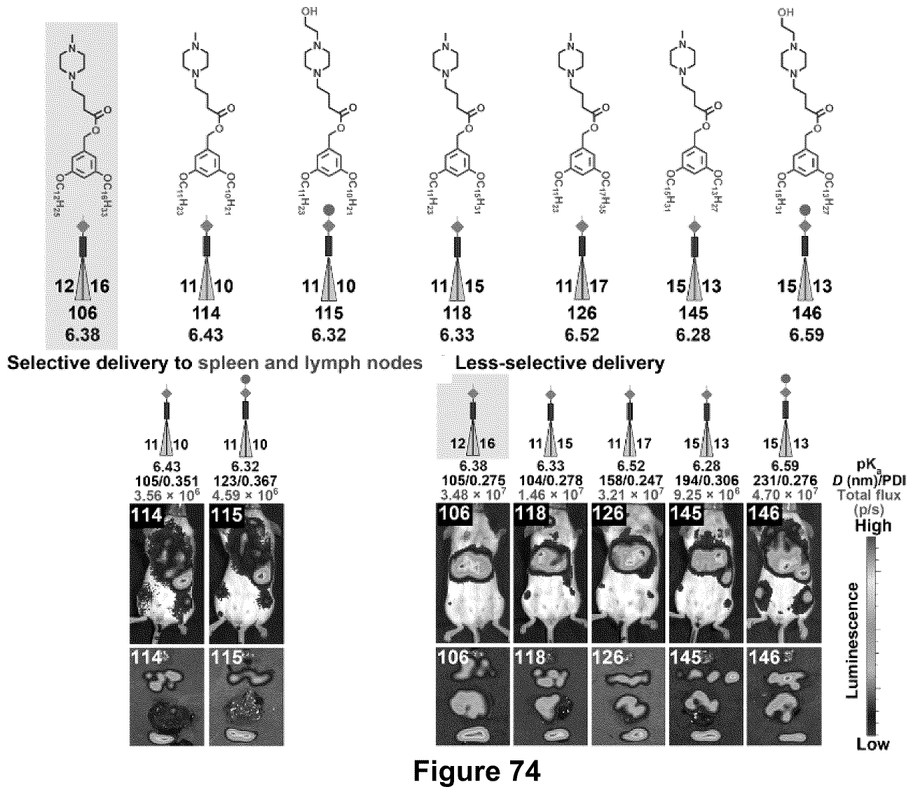

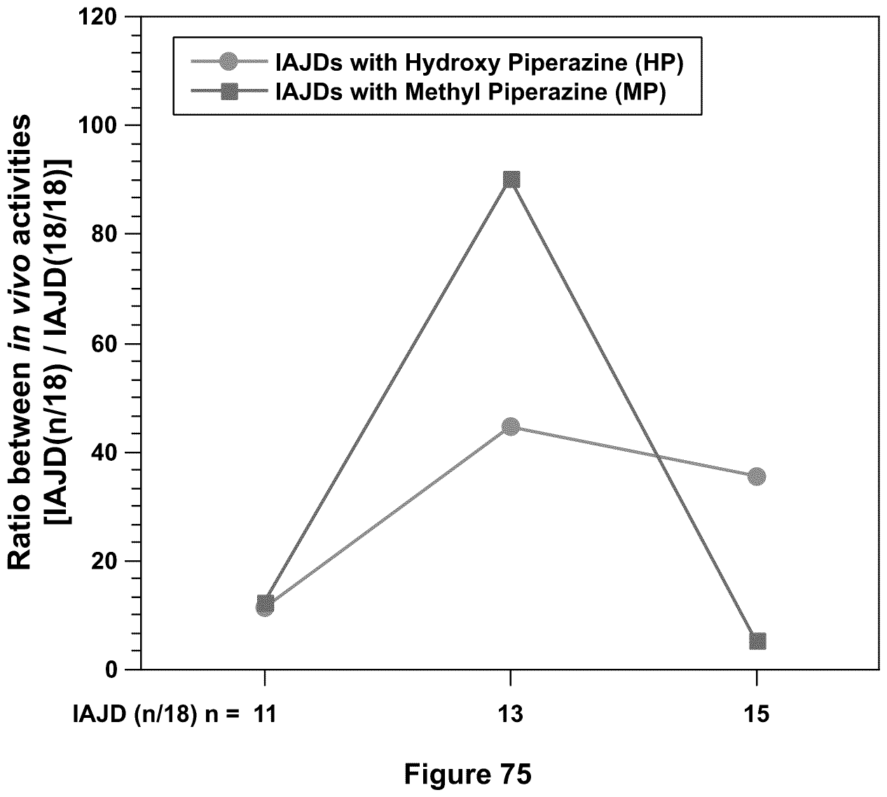

The invention relates in part to a one-component multifunctional sequence-defined IAJD delivery system that co-assembles with mRNA by simple injection into DNPs (FIG. 1, FIG. 59 through FIG. 62, and FIG. 73 through FIG. 75).

The materials and methods employed in these experiments are now described.

### Materials

3,5-Dihydroxybenzoic acid (Acros, 97%), (rac)-3-(bromomethyl)heptane, 2-ethylhexyl bromide, (Aldrich, 95%), 1-bromooctane (Aldrich, 99%), 1-bromononane (Lancaster, 99%), 1-bromodecane (Acros, 98%), 1-bromoundecane (Aldrich, 99%), 1-bromododecane (Alfa Aesar, 99%), 1bromotetradecane (Acros, 98%), 1-bromopentadecane (Aldrich, 98%), 1-bromohexadecane (TCI, 96%), 1-bromooctadecane (Acros, 96%), 1-heptadecanol (TCI, 97%), benzyl chloride (Alfa Aesar, 99%), 4toluenesulfonyl chloride (Alfa Aesar, 98%), palladium on activated carbon catalyst (Spectrum, 10 wt % loading), lithium aluminium hydride (LiAlH₄, TCI, 95%), 4-bromobutyric acid (Acros, 98%), thionyl chloride (Alfa Aesar, 99+%), potassium phthalimide (Chem Impex, 99.8%), 1-methylpiperazine (Alfa Aesar, 98%), 1-(2-hydroxyethyl)piperazine (Acros, 99%), triethylamine (TCI, 99%), other reagents and solvents for chemical synthesis were obtained from commercial sources and were used as received. CH2Cl2 (DCM) was dried over CaH2 and distilled freshly before use. 4-(Dimethylamino)pyridinium 4toluenesulfonate (DPTS) was prepared according to a literature procedure (Moore and Stupp, Macromolecules 1990, 23, 65-70). Acetate buffer (10 mM) was prepared by dissolving sodium acetate (2.3 mM) and acetic acid (7.7 mM) in ultra-pure water. Final pH of the buffer was adjusted with 0.1 M HCl or 0.1 M NaOH solution. Nucleoside-modified messenger RNA encoding firefly luciferase (Luc-mRNA) was produced as previously described. Human embryonic kidney (HEK) 293T cells (American Type Culture Collection) were cultured in Dulbecco's modified Eagle's medium (DMEM) supplemented with 10% inactivated fetal bovine serum (FBS) (Gemini Bio-Products), 2 mM L-glutamine and 100 U/mL penicillin/streptomycin (Life Technologies). DPBS (Corning), OptiMEM (Gibco), UltraPure DNase/RNase-Free Distilled Water (Invitrogen), Trypsin-EDTA (0.25%, Gibco), Trypan Blue (Sigma-Aldrich), Cell Culture Lysis 5× Reagent (Promega), Luciferase Assay System (Promega) and D-luciferin sodium salt (Regis Technologies) were used as received.

### 2. Methods and Techniques

The purity and structural identity of intermediate compounds and final products were determined by a combination of characterization techniques including thin-layer chromatography (TLC), ¹H and ¹³C NMR, high-pressure liquid chromatography (HPLC) and matrix assisted laser desorption ionization-time of flight (MALDI-TOF) mass spectrometry. ¹H and ¹³C NMR spectra were recorded at 400 MHz and 101 MHz respectively, on a Bruker NEO (400 MHz) NMR spectrometer with autosampler, or 500 MHz and 126 MHz respectively, on a Bruker DRX (500 MHz) NMR spectrometer. All NMR spectra were measured at 23° C. in CDCl₃. Residual protic solvent of CDCl₃ (¹H, δ 7.26 ppm; ¹³C, δ 77.16 ppm) and tetramethylsilane (TMS, δ 0 ppm) are used as the internal reference in the ¹H and ¹³C NMR spectra. NMR spectra were analyzed by MNova 14 or TopSpin 4.07 (Bruker). TLC was used to monitor the evolution of the reaction by using silica gel 60 F254 precoated plates (E. Merck). Individual compounds with aromatic groups were visualized by UV light (λ=254 nm). For compounds without aromatic groups, the TLC plate was stained with iodine vapor to help visualization. Purification by flash column chromatography (SiO₂) was performed using silica gel from Silicycle (60 Å, 40-63 μm) with the indicated eluent. The determination of the purity of individual compounds by high-pressure liquid chromatography (HPLC) was performed by using Shimadzu LC20AD high-performance liquid chromatograph pump, a PE Nelson Analytical 900 Series integration data station, a Shimadzu SPD-10A VP (UV-vis, λ=254 nm) and three AM gel columns (a guard column, two 500 Å, 10 pm columns). THF with 5% of NEt₃ was used as the solvent and the characterization was carried out at 23° C. Detection of compounds was determined by UV absorbance (λ=254 nm) or RI (refractive index) detector. The molar mass of molecules was determined by MALDI-TOF mass spectrometry on a PerSeptive Biosystem-Voyager-DE (Framingham, MA) mass spectrometer equipped with nitrogen laser (337 nm) and operating in linear mode. Angiotensin II and Bombesin were used as standards for internal calibration. For the preparation of sample solution, compound for analysis was dissolved in THF (5-10 mg/mL) firstly. Then the matrix (2,5-dihydroxybenzoic acid) was dissolved in THF 10 mg/mL. Finally, the above two solutions were mixed with a 1/5 (v/v, compound solution/matrix solution) ratio. Then one drop of sample solution was placed on the MALDI plate and dried at 23° C. Afterwards, the plate was inserted into the vacuum chamber of the instrument for analysis. The laser intensity and voltages applied for the analysis were adjusted based on the molar mass and nature of each analyzed compound.

Dynamic Light Scattering (DLS) for the dimensions (sizes and polydispersities) of DNPs was performed on a Malvern Instruments particle sizer (Zetasizer Nano S, Malvern Instruments, UK) equipped with 4 mW He—Ne laser 633 nm and avalanche photodiode positioned at 175° to the beam and temperature-controlled cuvette holder. Instrument parameters were set up automatically along with measurement times. Sample solution (c.a. 0.4 mL) was placed in a semi-micro cuvette (1.6 mL, polystyrene, 10×10×45 mm, Greiner Bio-One) and the measurements were performed at 23° C.

For pKₐ measurements of individual IAJD molecules, the IAJD molecules were dissolved in ethanol (saturated with NaCl) at a concentration of 1.5 mg/mL and in a volume of 3 mL. Then 0.1 M HCl solution was added to the above ethanol solution with an increment of 7.5 μL. The resulting pH after each addition of HCl solution was measured by an Thermo Scientific Orion Star A121 meter with Thermo Scientific Orion 8220BNWP pH probe. pKₐ was determined using the half equivalence point titration.

Formulation of DNPs Co-assembled from IAJDs and Luc-mRNA: nucleoside-modified mRNA encoding firefly luciferase (Luc-mRNA) was dissolved in UltraPure DNase/RNase-free distilled water with an initial concentration of 4.0 mg/mL. IAJD molecules were dissolved in ethanol with an initial concentration of 80 mg/mL. 12.5 μL of Luc-mRNA solution was taken and placed into a clean RNAs free eppendorf (1.5 mL). Then 463 μL of acetate buffer (10 mM, pH 4.0) was added. Afterwards, 25 μL of IAJD in the ethanol stock solution was taken and rapidly injected into the above Luc-mRNA solution in acetate buffer followed by vortex for 5 seconds.

Luminescence Characterization for In Vitro Transfection Experiments: the determination of luminescence intensity for in vitro Luc-mRNA transfection experiments with HEK 293T Cells was performed using a MiniLumat LB 9506 luminometer (Berthold/EG&G; Wallac).

Luminescence Characterization for In Vivo Transfection Experiments: bioluminescence imaging was performed with an IVIS Spectrum imaging system (PerkinElmer, Waltham, MA). Mice were anesthetized with 3% of isoflurane (Piramal Healthcare Limited) and intraperitoneally (i.p.) administered with D-luciferin (Regis Technologies) at a dose of 150 mg/kg of body weight. Ten minutes post administration of D-luciferin, mice were placed on the imaging platform while being maintained on isoflurane via a nose cone and imaged using a certain exposure time (60, 30, or 15 seconds).

Bioluminescence values were quantified by measuring photon flux (photons/second, p/s) in the region of interest (ROI) on mice where bioluminescence signal emanated using the Living Image Software (PerkinElmer). To quantify luminescent flux, an oval ROI was placed over each organ of interest and analyzed.

In Vitro mRNA Delivery: for in vitro Luc-mRNA transfection screening experiments, human embryonic kidney 293T cells (HEK 293T) were seeded into 96-well plates (20,000 cells/well/200 μL) and cultured for 18-20 hours at 37° C., 5% CO2 in complete Dulbecco's modified Eagle's medium (DMEM) supplemented with 2 mM L-glutamine (Life Technologies), 100 U/mL penicillin/streptomycin (Invitrogen) and 10% fetal bovine serum (FBS, Gemini Bio-Products). Cells were transfected with DNPs with encapsulated Luc-mRNA at a constant concentration of Luc-mRNA, 125 ng per well. Luc-mRNA encapsulated in FDA approved MC3-based LNPs were used as positive controls for cell transfection, at the concentration of Luc-mRNA of 125 ng per well, the same as for tested DNPs. After transfection cells were further cultured at 37° C., 5% CO2 for 18-20 hours, then medium was aspirated and cells were lysed with luciferase cell culture lysis reagent (30 μL/well) (Promega, Madison, WI). For the determination of the luciferase enzymatic activity as luminescence, 2.5 μL of the lysed cells was mixed with 10 μL of firefly luciferase assay substrate (Luciferase assay system, Promega) and luminescence was analyzed by MiniLumat LB9506 luminometer. Transfections were performed in triplicate.

In Vivo mRNA Delivery in Mice with DNPs: All animals used were in accordance with the guidelines and approval from the Pennsylvania University Institution of Animal Care and Use Committee. Female or male BALB/c mice (6-8 weeks old, from Charles River Laboratories) were anesthetized with isoflurane (Piramal Healthcare Limited) and injected via retro-orbital sinus with 100 μL of DNP solution containing 10 pg of Luc-mRNA. At 4-7 hours post injection, mice were i.p. injected with D-Luciferin (150 mg/kg of body weight, Regis Technologies) and imaged on a PerkinElmer IVIS Spectrum CT system (PerkinElmer, Waltham, MA). Tissue luminescence signal was measured on the IVIS imaging system using a certain exposure time (60, 30 or 15 seconds) and medium binning (binning=8) to ensure that the signal obtained was within operative detection range. For IVIS imaging of the organs, mice were sacrificed, and heart, lungs, liver, and spleen were immediately collected, and bioluminescence imaging was performed as described above. Image analysis was conducted with the Living Image software (PerkinElmer). Bioluminescence values were quantified by measuring photon flux (photons/second) in the region of interest (ROI) using Living Image software.

Scheme 10 (below) shows the synthesis of undecane-based IAJDs with nonsymmetric alkyl chains.

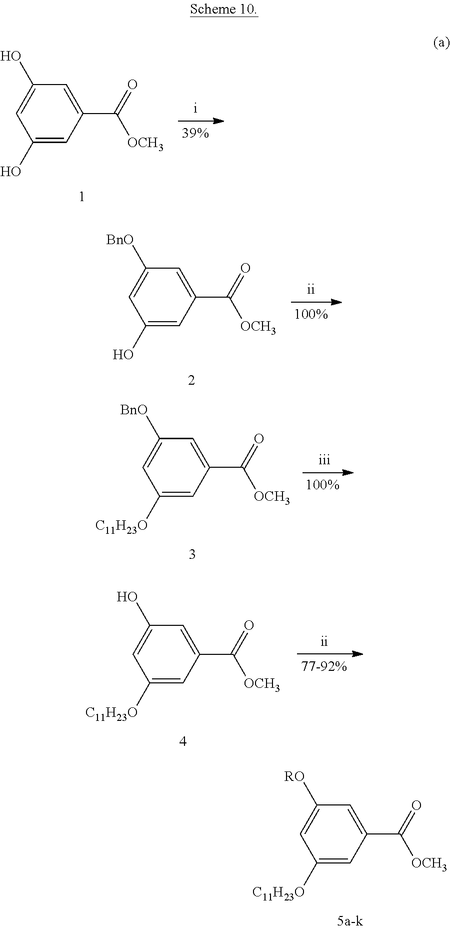

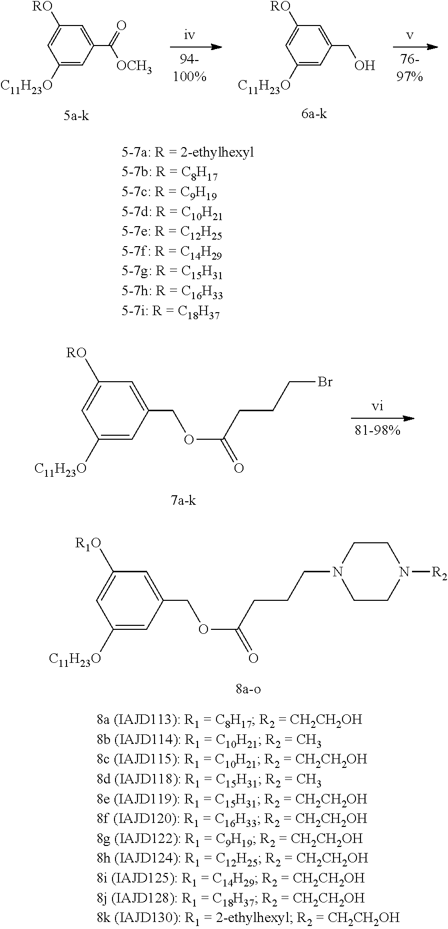

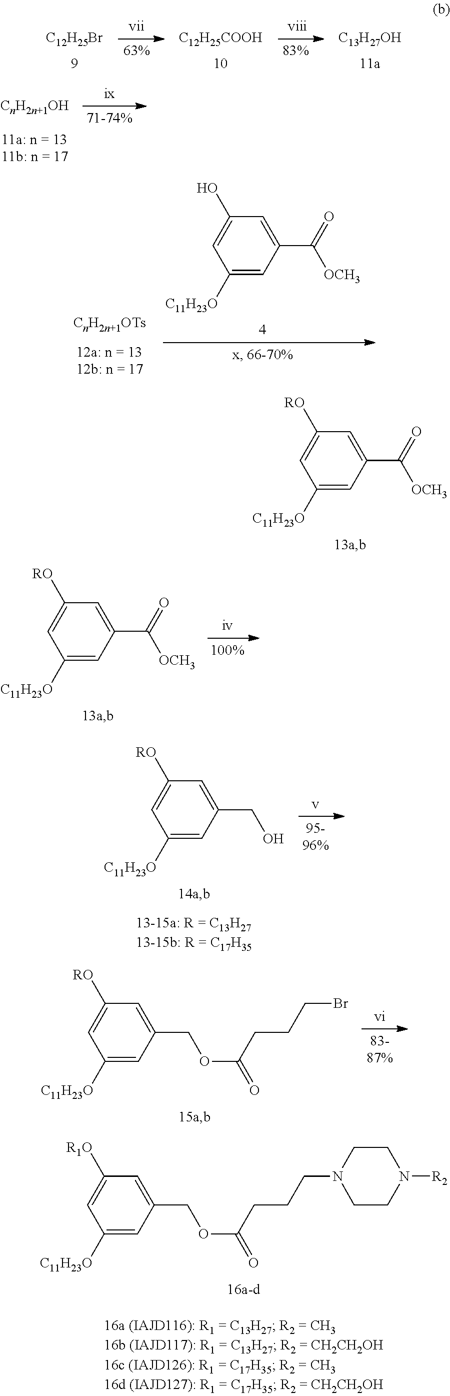

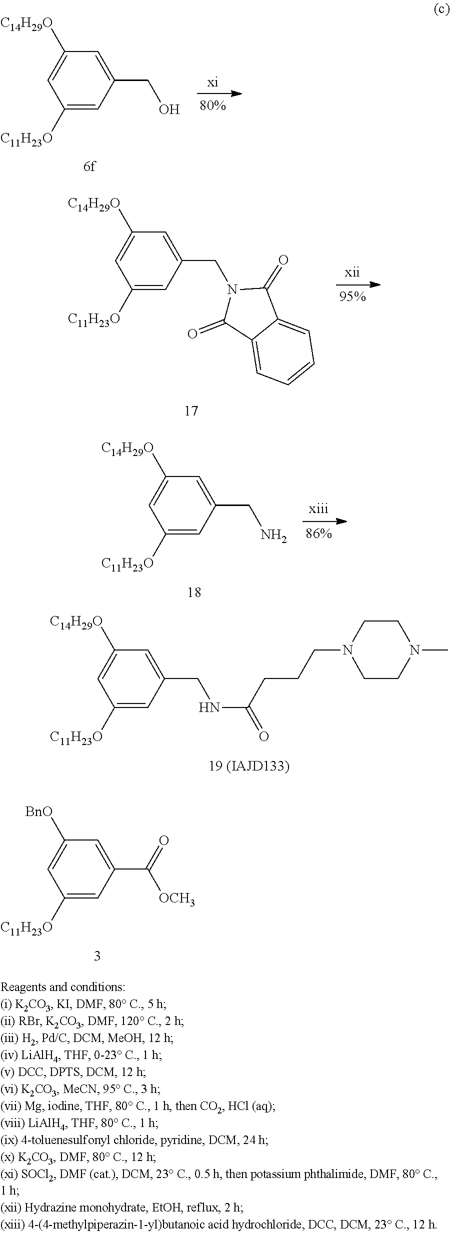

Methyl 3-(benzyloxy)-5-(undecyloxy)benzoate (3). The synthesis of methyl 3-(benzyloxy)-5-hydroxybenzoate (2) was adapted from literature procedures. In this work, the procedures were optimized and increased the scale of compound 2 to 23 g. Compound 2 (4.00 g, 15.49 mmol, 1 equiv), 1-bromoundecane (4.01 g, 17.04 mmol, 1.1 equiv) and K₂CO₃ (4.28 g, 30.98 mmol, 2 equiv) were stirred in DMF (50 mL). The mixture was stirred at 120° C. under N₂ atmosphere for 2 h. The reaction mixture was cooled to 23° C. and DMF was removed under reduced pressure. Then water (30 mL) was added, and the mixture was extracted by DCM (30 mL), three times. The organic phases were combined, dried over anhydrous MgSO₄ and filtered. The filtrate was concentrated and purified by column chromatography (SiO₂) with hexane/EtOAc=20/1 as the mobile phase to give the title compound as a colorless oil (6.39 g, 100%).

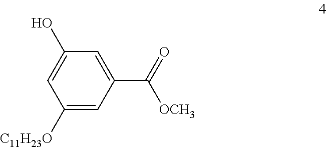

Methyl 3-hydroxy-5-(undecyloxy)benzoate (4). Compound 3 (6.39 g, 15.49 mmol) was dissolved in the mixture of DCM (40 mL) and methanol (20 mL). Then Pd/C (0.32 g, 5 wt %) was added and the flask was evacuated and filled with hydrogen, three times. The mixture was stirred at 23° C. for 12 h under hydrogen atmosphere. The reaction mixture was filtered through Celite, and the filter cake was washed with DCM carefully. Evaporation of the solvent yielded the title compound as a white solid (5.02 g, 100%).

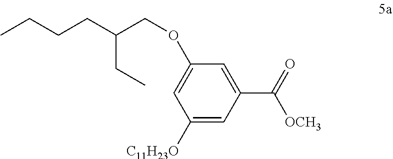

Methyl 3-((2-ethylhexyl)oxy)-5-(undecyloxy)benzoate (5a). Compound 5a was synthesized from compound 4 (1.00 g, 3.10 mmol, 1 equiv), 3-(bromomethyl)heptane (0.72 g, 3.72 mmol, 1.2 equiv) and K₂CO₃ (1.03 g, 7.45 mmol, 2.4 equiv) following a procedure similar to that used for the synthesis of compound 3. The title compound was obtained as a yellow oil (1.16 g, 92%).

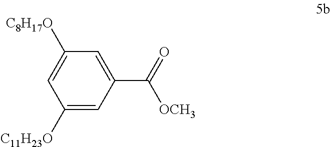

Methyl 3-(octyloxy)-5-(undecyloxy)benzoate (5b). Compound 4 (1.00 g, 3.10 mmol, 1 equiv), 1-bromooctane (0.63 g, 3.26 mmol, 1.05 equiv) and K₂CO₃ (0.87 g, 6.20 mmol, 2 equiv) were stirred in DMF (15 mL). The mixture was stirred at 120° C. under N₂ atmosphere for 2 h. After cooled to 23° C., the reaction mixture was poured into ice/water (100 mL) and the resulted white precipitates were filtered and collected. Then the precipitates were purified by recrystallization from a minimum amount of acetone to give the title compound as a white solid (1.10 g, 82%).

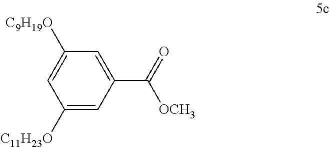

Methyl 3-(nonyloxy)-5-(undecyloxy)benzoate (5c). Compound 5c was synthesized from compound 4 (1.00 g, 3.10 mmol, 1 equiv), 1-bromononane (0.69 g, 3.31 mmol, 1.1 equiv) and K₂CO₃ (0.92 g, 6.63 mmol, 2.2 equiv) following a procedure similar to that used for the synthesis of compound 5b. The title compound was obtained as a white solid (1.11 g, 80%).

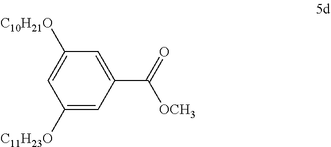

Methyl 3-(decyloxy)-5-(undecyloxy)benzoate (5d). Compound 5d was synthesized from compound 4 (1.00 g, 3.10 mmol, 1 equiv), 1-bromodecane (0.72 g, 3.26 mmol, 1.05 equiv) and K₂CO₃ (0.87 g, 6.20 mmol, 2 equiv) following a procedure similar to that used for the synthesis of compound 5b. The title compound was obtained as a white solid (1.25 g, 87%).

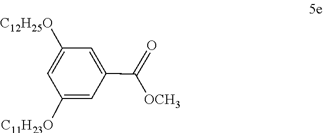

Methyl 3-(dodecyloxy)-5-(undecyloxy)benzoate (5e). Compound 5e was synthesized from compound 4 (1.00 g, 3.10 mmol, 1 equiv), 1-bromododecane (0.82 g, 3.30 mmol, 1.1 equiv) and K₂CO₃ (0.92 g, 6.63 mmol, 2.2 equiv) following a procedure similar to that used for the synthesis of compound 5b. The title compound was obtained as a white solid (1.25 g, 82%).

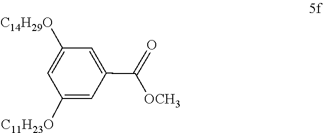

Methyl 3-(tetradecyloxy)-5-(undecyloxy)benzoate (5f). Compound 5f was synthesized from compound 4 (1.00 g, 3.10 mmol, 1 equiv), 1-bromotetradecane (0.90 g, 3.26 mmol, 1.05 equiv) and K₂CO₃ (0.87 g, 6.20 mmol, 2 equiv) following a procedure similar to that used for the synthesis of compound 5b. The title compound was obtained as a white solid (1.32 g, 82%).

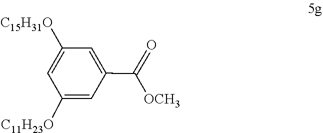

Methyl 3-(pentadecyloxy)-5-(undecyloxy)benzoate (5g). Compound 5g was synthesized from compound 4 (1.00 g, 3.10 mmol, 1 equiv), 1-bromopentadecane (0.95 g, 3.26 mmol, 1.05 equiv) and K₂CO₃ (0.87 g, 6.20 mmol, 2 equiv) following a procedure similar to that used for the synthesis of compound 5b. The title compound was obtained as a white solid (1.37 g, 83%).

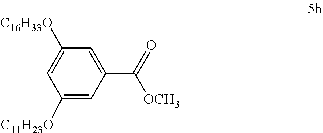

Methyl 3-(hexadecyloxy)-5-(undecyloxy)benzoate (5h). Compound 5h was synthesized from compound 4 (1.00 g, 3.10 mmol, 1 equiv), 1-bromohexadecane (1.01 g, 3.31 mmol, 1.1 equiv) and K₂CO₃ (0.92 g, 6.63 mmol, 2.2 equiv) following a procedure similar to that used for the synthesis of compound 5b. The title compound was obtained as a white solid (1.30 g, 77%).

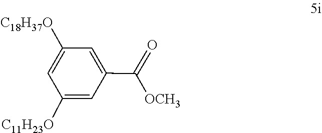

Methyl 3-(octadecyloxy)-5-(undecyloxy)benzoate (5i). Compound 5i was synthesized from compound 4 (1.00 g, 3.10 mmol, 1 equiv), 1-bromoctadecanene (1.09 g, 3.26 mmol, 1.05 equiv) and K₂CO₃ (0.87 g, 6.20 mmol, 2 equiv) following a procedure similar to that used for the synthesis of compound 5b. The title compound was obtained as a white solid (1.63 g, 92%).

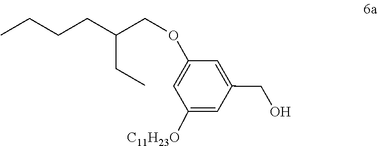

(3-((2-Ethylhexyl)oxy)-5-(undecyloxy)phenyl)methanol (6a). Compound 5a (1.14 g, 2.62 mmol, 1 equiv) was dissolved in 5 mL dry THF, which was added dropwise to a slurry of LiAlH₄ (78 mg, 2.06 mmol, 0.8 equiv) in dry THF (5 mL) at 0° C. under N₂ atmosphere. The resulted mixture was stirred at 23° C. for 1 h. The reaction was quenched by the successive addition of water (0.3 mL), 15% NaOH aqueous solution (0.3 mL) and water (1.5 mL). Then the white precipitates in the mixture were filtered out and the filtrate was dried over anhydrous MgSO₄. Filtration and evaporation of the solvent yielded the title compound as a colorless oil (1.02 g, 96%).

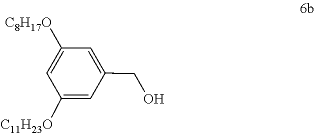

(3-(Octyloxy)-5-(undecyloxy)phenyl)methanol (6b). Compound 6b was synthesized from compound 5b (0.92 g, 2.12 mmol, 1 equiv) and LiAlH₄ (80 mg, 2.12 mmol, 1 equiv) following a procedure similar to that used for the synthesis of compound 6a. The title compound was obtained as a colorless oil (0.82 g, 95%).

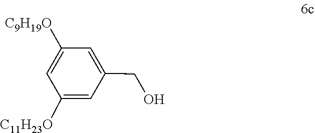

(3-(Nonyloxy)-5-(undecyloxy)phenyl)methanol (6c). Compound 6c was synthesized from compound 5c (1.00 g, 2.23 mmol, 1 equiv) and LiAlH₄ (85 mg, 2.23 mmol, 1 equiv) following a procedure similar to that used for the synthesis of compound 6a. The title compound was obtained as a colorless oil (0.88 g, 94%).

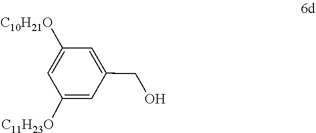

(3-(Decyloxy)-5-(undecyloxy)phenyl)methanol (6d). Compound 6d was synthesized from compound 5d (1.05 g, 2.27 mmol, 1 equiv) and LiAlH₄ (86 mg, 2.25 mmol, 1 equiv) following a procedure similar to that used for the synthesis of compound 6a. The title compound was obtained as a colorless oil (0.98 g, 98%).

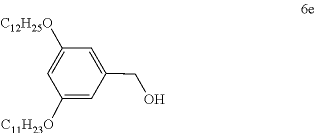

(3-(Dodecyloxy)-5-(undecyloxy)phenyl)methanol (6e). Compound 6e was synthesized from compound 5e (1.15 g, 2.34 mmol, 1 equiv) and LiAlH₄ (89 mg, 2.34 mmol, 1 equiv) following a procedure similar to that used for the synthesis of compound 6a. The title compound was obtained as a colorless oil (1.08 g, 99%).

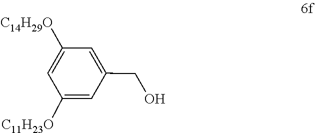

(3-(Tetradecyloxy)-5-(undecyloxy)phenyl)methanol (6f). Compound 6f was synthesized from compound 5f (1.15 g, 2.21 mmol, 1 equiv) and LiAlH₄ (84 mg, 2.21 mmol, 1 equiv) following a procedure similar to that used for the synthesis of compound 6a. The title compound was obtained as a colorless oil (1.08 g, 99%).

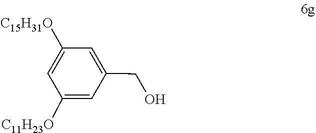

(3-(Pentadecyloxy)-5-(undecyloxy)phenyl)methanol (6g). Compound 6g was synthesized from compound 5g (1.12 g, 2.10 mmol, 1 equiv) and LiAlH₄ (79 mg, 2.10 mmol, 1 equiv) following a procedure similar to that used for the synthesis of compound 6a. The title compound was obtained as a white solid (1.06 g, 100%).

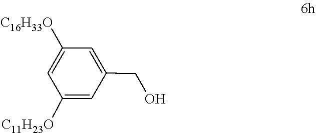

(3-(Hexadecyloxy)-5-(undecyloxy)phenyl)methanol (6h). Compound 6h was synthesized from compound 5h (1.10 g, 2.01 mmol, 1 equiv) and LiAlH₄ (76 mg, 2.01 mmol, 1 equiv) following a procedure similar to that used for the synthesis of compound 6a. The title compound was obtained as a white solid (1.03 g, 99%).

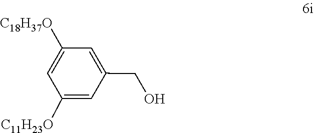

(3-(Octadecyloxy)-5-(undecyloxy)phenyl)methanol (6i). Compound 6i was synthesized from compound 5i (1.15 g, 2.05 mmol, 1 equiv) and LiAlH₄ (78 mg, 2.05 mmol, 1 equiv) following a procedure similar to that used for the synthesis of compound 6a. The title compound was obtained as a white solid (1.32 g, 94%).

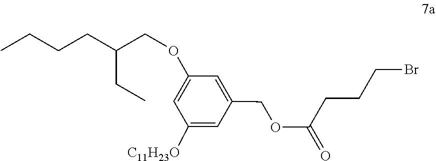

3-((2-Ethylhexyl)oxy)-5-(undecyloxy)benzyl 4-bromobutanoate (7a). Compound 6a (0.50 g, 1.23 mmol, 1 equiv), 4-bromobutyric acid (0.23 g, 1.35 mmol, 1.1 equiv) and DPTS (0.40 g, 1.35 mmol, 1.1 equiv) were dissolved in 8 mL DCM. N,N′-Dicyclohexylcarbodiimide (DCC, 0.51 g, 2.46 mmol, 2 equiv) was added in one portion into the above mixture. The reaction mixture was allowed to stir at 23° C. for 12 h. The reaction mixture was filtered to remove the urea, which was washed with DCM carefully. The filtrate was concentrated and purified by column chromatography (SiO₂) with hexane/DCM=1/1 as the eluent to give the title compound as a colorless oil (0.61 g, 90%).

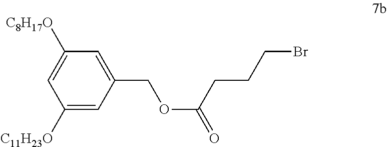

3-(Octyloxy)-5-(undecyloxy)benzyl 4-bromobutanoate (7b). Compound 7b was synthesized from compound 6b (0.40 g, 0.98 mmol, 1 equiv), 4-bromobutyric acid (0.18 g, 1.08 mmol, 1.1 equiv), DPTS (0.32 g, 1.08 mmol, 1.1 equiv) and DCC (0.41 g, 1.97 mmol, 2 equiv) following a procedure similar to that used for the synthesis of compound 7a. The title compound was obtained as a colorless oil (0.53 g, 97%).

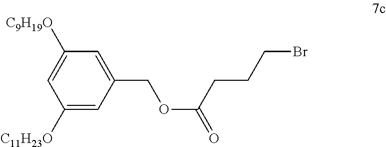

3-(Nonyloxy)-5-(undecyloxy)benzyl 4-bromobutanoate (7c). Compound 7c was synthesized from compound 6c (0.40 g, 0.95 mmol, 1 equiv), 4-bromobutyric acid (0.16 g, 0.95 mmol, 1 equiv), DPTS (0.28 g, 0.95 mmol, 1 equiv) and DCC (0.39 g, 1.90 mmol, 2 equiv) following a procedure similar to that used for the synthesis of compound 7a. The title compound was obtained as a colorless oil (0.47 g, 87%).

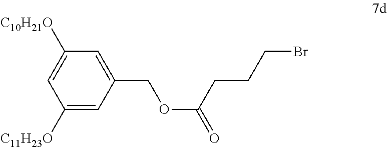

3-(Decyloxy)-5-(undecyloxy)benzyl 4-bromobutanoate (7d). Compound 7d was synthesized from compound 6d (0.40 g, 0.92 mmol, 1 equiv), 4-bromobutyric acid (0.17 g, 1.01 mmol, 1.1 equiv), DPTS (0.30 g, 1.01 mmol, 1.1 equiv) and DCC (0.38 g, 1.84 mmol, 2 equiv) following a procedure similar to that used for the synthesis of compound 7a. The title compound was obtained as a colorless oil (0.51 g, 95%).

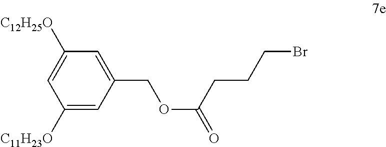

3-(Dodecyloxy)-5-(undecyloxy)benzyl 4-bromobutanoate (7e). Compound 7e was synthesized from compound 6e (0.40 g, 0.86 mmol, 1 equiv), 4-bromobutyric acid (0.16 g, 0.95 mmol, 1.1 equiv), DPTS (0.28 g, 0.95 mmol, 1.1 equiv) and DCC (0.36 g, 1.73 mmol, 2 equiv) following a procedure similar to that used for the synthesis of compound 7a. The title compound was obtained as a colorless oil (0.50 g, 95%).

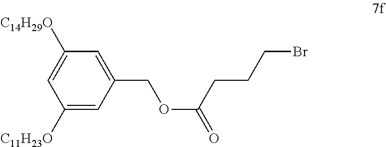

3-(Tetradecyloxy)-5-(undecyloxy)benzyl 4-bromobutanoate (7f). Compound 7f was synthesized from compound 6f (0.40 g, 0.82 mmol, 1 equiv), 4-bromobutyric acid (0.15 g, 0.90 mmol, 1.1 equiv), DPTS (0.26 g, 0.90 mmol, 1.1 equiv) and DCC (0.34 g, 1.63 mmol, 2 equiv) following a procedure similar to that used for the synthesis of compound 7a. The title compound was obtained as a colorless oil (0.45 g, 86%).

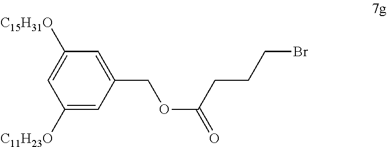

3-(Pentadecyloxy)-5-(undecyloxy)benzyl 4-bromobutanoate (7g). Compound 7g was synthesized from compound 6g (0.40 g, 0.79 mmol, 1 equiv), 4-bromobutyric acid (0.145 g, 0.87 mmol, 1.1 equiv), DPTS (0.256 g, 0.87 mmol, 1.1 equiv) and DCC (0.33 g, 1.58 mmol, 2 equiv) following a procedure similar to that used for the synthesis of compound 7a. The title compound was obtained as a colorless oil (0.48 g, 93%).

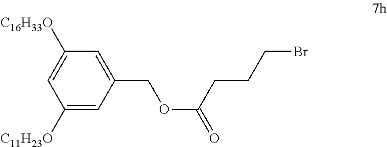

3-(Hexadecyloxy)-5-(undecyloxy)benzyl 4-bromobutanoate (7h). Compound 7h was synthesized from compound 6h (0.40 g, 0.77 mmol, 1 equiv), 4-bromobutyric acid (0.13 g, 0.77 mmol, 1 equiv), DPTS (0.23 g, 0.77 mmol, 1 equiv) and DCC (0.32 g, 1.54 mmol, 2 equiv) following a procedure similar to that used for the synthesis of compound 7a. The title compound was obtained as a colorless oil (0.39 g, 76%).

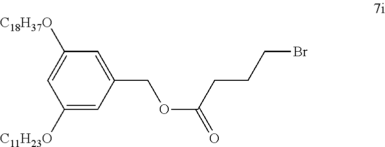

3-(Octadecyloxy)-5-(undecyloxy)benzyl 4-bromobutanoate (7i). Compound 7i was synthesized from compound 6i (0.40 g, 0.73 mmol, 1 equiv), 4-bromobutyric acid (0.14 g, 0.81 mmol, 1.1 equiv), DPTS (0.24 g, 0.81 mmol, 1.1 equiv) and DCC (0.30 g, 1.43 mmol, 2 equiv) following a procedure similar to that used for the synthesis of compound 7a. The title compound was obtained as a white solid (0.49 g, 97%).

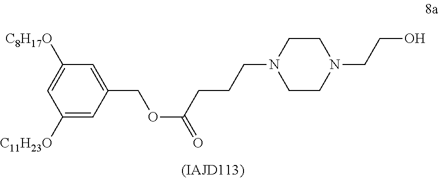

3-(Octyloxy)-5-(undecyloxy)benzyl 4-(4-(2-hydroxyethyl)piperazin-1-yl)butanoate (8a, IAJD113). Compound 7b (0.25 g, 0.45 mmol, 1.0 equiv), 1-(2-hydroxyethyl)piperazine (61 mg, 0.47 mmol, 1.05 equiv), K₂CO₃ (65 mg, 0.47 mmol, 1.05 equiv) were stirred in MeCN (15 mL). The mixture was heated at 95° C. for 3 h. The reaction mixture was cooled to 23° C. and solvent was removed under reduced pressure. Then water (20 mL) was added, and the resulted mixture was extracted by DCM (20 mL) for three times. The organic phase was combined, dried over anhydrous MgSO₄, filtered and dried to give the crude product. The crude product was purified by column chromatography (SiO₂) with DCM/MeOH=30/1 and 15/1 as the mobile phase. Then the obtained product was dissolved in DCM (20 mL), which was washed by NaHCO₃solution (2%, 20 mL). The aqueous phase was extracted by DCM (20 mL) for another two times. The organic phase was collected and dried over anhydrous MgSO₄. Filtration and evaporation of the solvent yielded the title compound as a light-yellow oil (0.25 g, 93%).

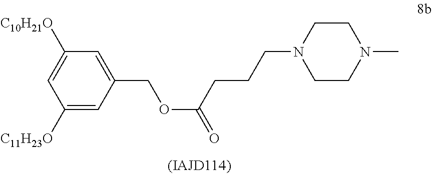

3-(Decyloxy)-5-(undecyloxy)benzyl 4-(4-methylpiperazin-1-yl)butanoate (8b, IAJD114). Compound 8b was synthesized from compound 7d (0.24 g, 0.41 mmol), 1-methylpiperazine (49 mg, 0.49 mmol) and K₂CO₃ (68 mg, 0.49 mmol) following a procedure similar to that used for the synthesis of compound 8a. The title compound was obtained as a light-yellow oil (0.24 g, 88%).

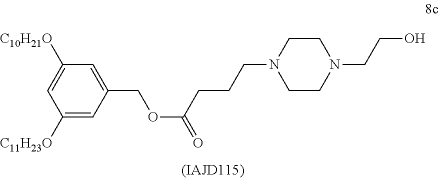

3-(Decyloxy)-5-(undecyloxy)benzyl 4-(4-(2-hydroxyethyl)piperazin-1-yl)butanoate (8c, IAJD115). Compound 8c was synthesized from compound 7d (0.24 g, 0.41 mmol), 1-(2-hydroxyethyl)piperazine (64 mg, 0.49 mmol) and K₂CO₃ (68 mg, 0.49 mmol) following a procedure similar to that used for the synthesis of compound 8a. The title compound was obtained as a light-yellow oil (0.24 g, 92%).

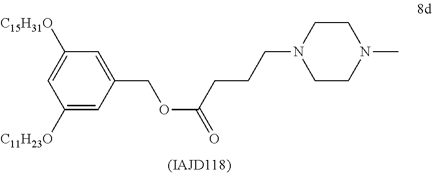

3-(Pentadecyloxy)-5-(undecyloxy)benzyl 4-(4-methylpiperazin-1-yl)butanoate (8d, IAJD118). Compound 8d was synthesized from compound 7g (0.22 g, 0.34 mmol), 1-methylpiperazine (38 mg, 0.37 mmol) and K₂CO₃ (70 mg, 0.51 mmol) following a procedure similar to that used for the synthesis of compound 8a. The title compound was obtained as a colorless oil (0.20 g, 87%).

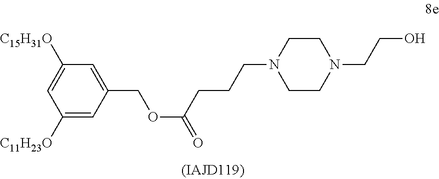

3-(Pentadecyloxy)-5-(undecyloxy)benzyl 4-(4-(2-hydroxyethyl)piperazin-1-yl)butanoate (8e, IAJD119). Compound 8e was synthesized from compound 7g (0.20 g, 0.30 mmol), 1-(2-hydroxyethyl)piperazine (43 mg, 0.33 mmol) and K₂CO₃ (62 mg, 0.45 mmol) following a procedure similar to that used for the synthesis of compound 8a. The title compound was obtained as a colorless oil (0.16 g, 81%).

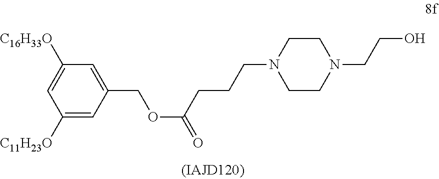

3-(Hexadecyloxy)-5-(undecyloxy)benzyl 4-(4-(2-hydroxyethyl)piperazin-1-yl)butanoate (8f, IAJD120). Compound 8f was synthesized from compound 7h (0.20 g, 0.30 mmol), 1-(2-hydroxyethyl)piperazine (51 mg, 0.39 mmol) and K₂CO₃ (57 mg, 0.41 mmol) following a procedure similar to that used for the synthesis of compound 8a. The title compound was obtained as a light-yellow oil (0.21 g, 98%).

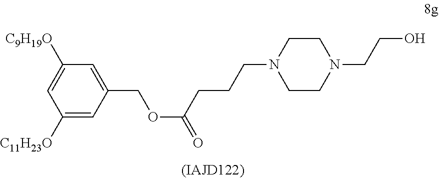

3-(Nonyloxy)-5-(undecyloxy)benzyl 4-(4-(2-hydroxyethyl)piperazin-1-yl)butanoate (8g, IAJD122). Compound 8g was synthesized from compound 7c (0.13 g, 0.23 mmol), 1-(2-hydroxyethyl)piperazine (31 mg, 0.24 mmol) and K₂CO₃ (33 mg, 0.24 mmol) following a procedure similar to that used for the synthesis of compound 8a. The title compound was obtained as a light-yellow oil (0.12 g, 86%).

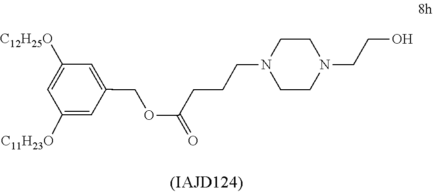

3-(Dodecyloxy)-5-(undecyloxy)benzyl 4-(4-(2-hydroxyethyl)piperazin-1-yl)butanoate (8h, IAJD124). Compound 8h was synthesized from compound 7e (0.20 g, 0.33 mmol), 1-(2-hydroxyethyl)piperazine (64 mg, 0.49 mmol) and K₂CO₃ (68 mg, 0.49 mmol) following a procedure similar to that used for the synthesis of compound 8a. The title compound was obtained as a light-yellow oil (0.20 g, 93%).

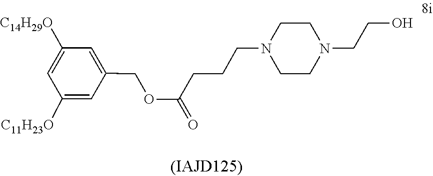

3-(Tetradecyloxy)-5-(undecyloxy)benzyl 4-(4-(2-hydroxyethyl)piperazin-1-yl)butanoate (8i, IAJD125). Compound 8i was synthesized from compound 7f (0.20 g, 0.31 mmol), 1-(2-hydroxyethyl)piperazine (45 mg, 0.34 mmol) and K₂CO₃ (47 mg, 0.34 mmol) following a procedure similar to that used for the synthesis of compound 8a. The title compound was obtained as a light-yellow oil (0.19 g, 88%).

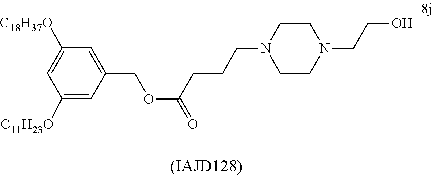

3-(Octadecyloxy)-5-(undecyloxy)benzyl 4-(4-(2-hydroxyethyl)piperazin-1-yl)butanoate (8j, IAJD128). Compound 8j was synthesized from compound 7i (0.24 g, 0.41 mmol), 1-(2-hydroxyethyl)piperazine (64 mg, 0.49 mmol) and K₂CO₃ (68 mg, 0.49 mmol) following a procedure similar to that used for the synthesis of compound 8a. The title compound was obtained as a light-yellow oil (0.23 g, 90%₀).

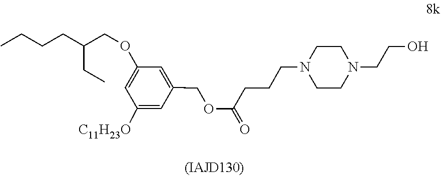

3-((2-Ethylhexyl)oxy)-5-(undecyloxy)benzyl 4-(4-(2-hydroxyethyl)piperazin-1-yl)butanoate (8k, IAJD130). Compound 8k was synthesized from compound 7a (0.24 g, 0.43 mmol), 1-(2-hydroxyethyl)piperazine (68 mg, 0.52 mmol) and K₂CO₃ (72 mg, 0.52 mmol) following a procedure similar to that used for the synthesis of compound 8a. The title compound was obtained as a light-yellow oil (0.24 g, 92%).

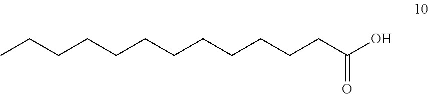

Tridecanoic acid (10, C₁₂H₂₅COOH). To a 250 mL two-necked round bottom flask, magnesium (1.00 g, 40 mmol) and catalytic amount of iodine (1 mg) were added. 1-Bromododecane (10.00 g, 40 mmol) in 50 mL of dry THF was mixed in an additional funnel and installed to one of the necks of the flask. The other neck of flask was connected to a condenser under N₂. Part solution of 1-bromododecane in THF (10 mL) was added to the flask, and the reaction mixture was heated at 80° C. Once the color of iodine disappeared and reaction mixture became cloudy, the rest of THF solution of 1-bromododecane was added dropwise. The reaction mixture was maintained at 80° C. for 1 hour until most of the magnesium was consumed. The reaction mixture was cooled to 23° C. and poured onto crushed dry ice (100 g). Once all the dry ice disappeared, hydrochloride acid (2M) was added to the reaction mixture until pH=1. The mixture was extracted with EtOAc (50 mL×3), dried with anhydrous MgSO₄, filtered and concentrated, and then purified by column chromatography on silica gel with a mobile phase of hexane/EtOAc=10/1 (v/v) to afford the title compound as a colorless oil (5.40 g, 63%).

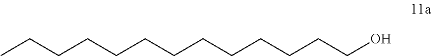

Tridecan-1-ol (11a, C₁₃H₂₇OH). Compound 10 (13.00 g, 61 mmol) were dissolved in dry THF (50 mL), which was added dropwise to a slurry of LiAlH₄ (3.60 g, 91 mmol) in dry THF under N₂ atmosphere. The resulted mixture was reflux at 80° C. for 1 h. The reaction was quenched by the successive addition of water, 15% NaOH aqueous solution and water. Then the mixture was filtered and dried over anhydrous MgSO₄. Filtration and evaporation of the solvent yielded the title compound as a colorless oil (10.10 g, 83%) without further purification.

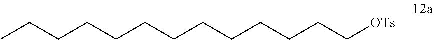 [SMILES: CCCCCCCCCCCCCOS(=O)(=O)c1ccc(C)cc1]

Tridecyl 4-methylbenzenesulfonate (12a, C₁₃H₂₇OTs). Compound 11a (10.10 g, 50 mmol) and pyridine (6.00 g, 75.60 mmol) were dissolved in dry DCM (50 mL), which was added 4-toluenesulfonyl chloride (10.50 g, 55.10 mmol) in dry DCM at 23° C. The resulted mixture was stirred at 23° C. for 24 h. Ethyl acetate (50 mL) was added, and the precipitates were removed by filtration. The filtrate was washed with saturated NaHCO₃solution, hydrochloride acid (2M), and brine, dried with anhydrous MgSO₄, filtered and concentrated. Cold methanol (100 mL) was added to precipitate the title compound as a white solid without further purification (12.60 g, 71%).

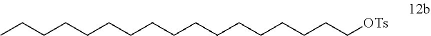 [SMILES: CCCCCCCCCCCCCCCCCOS(=O)(=O)c1ccc(C)cc1]

Heptadecyl 4-methylbenzenesulfonate (12b, C₁₇H₃₅OTs). Compound 12b was synthesized from 1-heptadecanol (7.00 g, 27.30 mmol), 4-toluenesulfonyl chloride (6.76 g, 35.50 mmol) and pyridine (3.00 g, 37.80 mmol) following a procedure similar to that used for the synthesis of compound 12a. The title compound was obtained as a white solid (8.30 g, 74%).

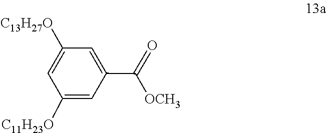

Methyl 3-(tridecyloxy)-5-(undecyloxy)benzoate (13a). Compound 4 (1.00 g, 3.10 mmol, 1 equiv), C₁₃H₂₇OTs (12a, 1.16 g, 3.26 mmol, 1.05 equiv) and K₂CO₃ (0.87 g, 6.20 mmol, 2 equiv) were stirred in DMF (10 mL). The mixture was stirred at 80° C. under N₂ atmosphere for 12 h. After cooled to 23° C., the reaction mixture was poured into ice/water (100 mL) and the resulted white precipitates were filtered and collected. Then the precipitates were purified by recrystallization from minimum acetone to give the title compound as a white solid (1.03 g, 66%).

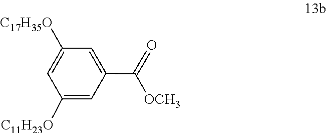

Methyl 3-(heptadecyloxy)-5-(undecyloxy)benzoate (13b). Compound 13b was synthesized from compound 4 (1.00 g, 3.10 mmol), C₁₇H₃₅OTs (12b, 1.34 g, 3.26 mmol) and K₂CO₃ (0.87 g, 6.20 mmol) following a procedure similar to that used for the synthesis of compound 13a. The title compound was obtained as a white solid (1.20 g, 70%).

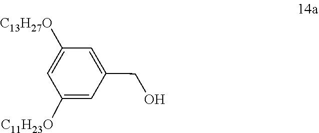

(3-(Tridecyloxy)-5-(undecyloxy)phenyl)methanol (14a). Compound 13a (0.90 g, 1.78 mmol, 1 equiv) was dissolved in 5 mL dry THF, which was added dropwise to a slurry of LiAlH₄ (67 mg, 1.78 mmol, 1 equiv) in dry THF (5 mL) at 0° C. under N₂ atmosphere. The resulted mixture was stirred at 23° C. for 1 h. The reaction was quenched by the successive addition of water (0.3 mL), 15% NaOH aqueous solution (0.3 mL) and water (1.5 mL). Then the white precipitates in the mixture were filtered out and the filtrate was dried over anhydrous MgSO₄. Filtration and evaporation of the solvent yielded the title compound as a colorless oil (0.85 g, 100%).

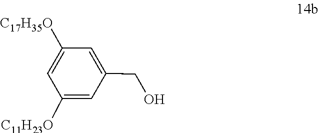

(3-(Heptadecyloxy)-5-(undecyloxy)phenyl)methanol (14b). Compound 14b was synthesized from compound 13b (1.15 g, 2.05 mmol, 1 equiv) and LiAlH₄ (78 mg, 2.05 mmol, 1 equiv) following a procedure similar to that used for the synthesis of compound 14a. The title compound was obtained as a white solid (1.09 g, 100%).

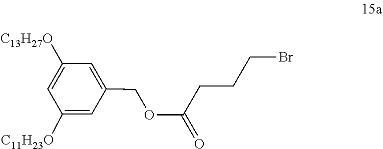

3-(Tridecyloxy)-5-(undecyloxy)benzyl 4-bromobutanoate (15a). Compound 14a (0.40 g, 0.84 mmol, 1 equiv), 4-bromobutyric acid (0.15 g, 0.92 mmol, 1.1 equiv) and DPTS (0.27 g, 0.92 mmol, 1.1 equiv) were dissolved in 8 mL DCM. DCC (0.35 g, 1.68 mmol, 2 equiv) was added in one portion into the above mixture. The reaction mixture was allowed to stir at 23° C. for 12 h. The reaction mixture was filtered to remove the urea, which was washed with DCM carefully. The filtrate was concentrated and purified by column chromatography (SiO₂) with Hexane/DCM=1/1 as the mobile phase to give the title compound as a colorless oil (0.50 g, 95%).

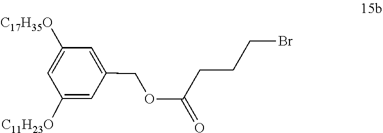

3-(Heptadecyloxy)-5-(undecyloxy)benzyl 4-bromobutanoate (15b). Compound 15b was synthesized from compound 14b (0.40 g, 0.75 mmol, 1 equiv), 4-bromobutyric acid (0.14 g, 0.83 mmol, 1.1 equiv), DPTS (0.26 g, 0.83 mmol, 1.1 equiv) and DCC (0.31 g, 1.50 mmol, 2 equiv) following a procedure similar to that used for the synthesis of compound 15a. The title compound was obtained as a white solid (0.49 g, 96%).

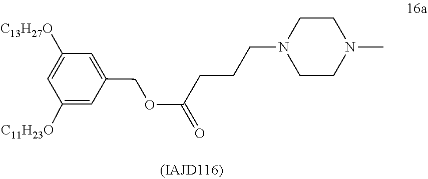

3-(Tridecyloxy)-5-(undecyloxy)benzyl 4-(4-methylpiperazin-1-yl)butanoate (16a, IAJD116). Compound 16a was synthesized from compound 15a (0.26 g, 0.36 mmol), 1-methylpiperazine (43 mg, 0.43 mmol) and K₂CO₃ (60 mg, 0.43 mmol) following a procedure similar to that used for the synthesis of compound 8a. The title compound was obtained as a light-yellow oil (0.19 g, 83%).

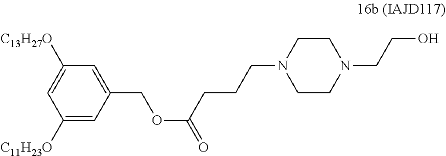

3-(Tridecyloxy)-5-(undecyloxy)benzyl 4-(4-(2-hydroxyethyl)piperazin-1-yl)butanoate (16b, IAJD117). Compound 16b was synthesized from compound 15a (0.20 g, 0.31 mmol), 1-(2-hydroxyethyl)piperazine (56 mg, 0.43 mmol) and K₂CO₃ (60 mg, 0.43 mmol) following a procedure similar to that used for the synthesis of compound 8a. The title compound was obtained as a light-yellow oil (0.18 g, 86%).

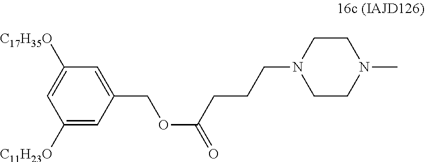

3-(Heptadecyloxy)-5-(undecyloxy)benzyl 4-(4-methylpiperazin-1-yl)butanoate (16c, IAJD126). Compound 16c was synthesized from compound 15b (0.22 g, 0.33 mmol), 1-methylpiperazine (36 mg, 0.36 mmol) and K₂CO₃ (68 mg, 0.50 mmol) following a procedure similar to that used for the synthesis of compound 8a. The title compound was obtained as a colorless oil (0.20 g, 87%).

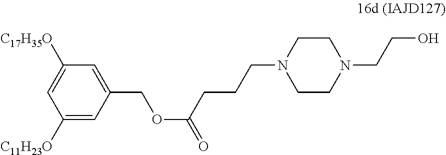

3-(Heptadecyloxy)-5-(undecyloxy)benzyl 4-(4-(2-hydroxyethyl)piperazin-1-yl)butanoate (16d, IAJD127). Compound 16d was synthesized from compound 15b (0.21 g, 0.30 mmol), 1-(2-hydroxyethyl)piperazine (40 mg, 0.33 mmol) and K₂CO₃ (62 mg, 0.45 mmol) following a procedure similar to that used for the synthesis of compound 8a. The title compound was obtained as a colorless oil (0.20 g, 87%).

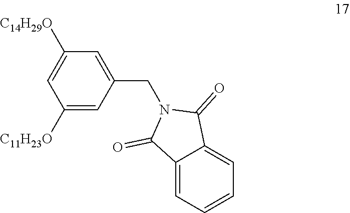

2-(3-(Tetradecyloxy)-5-(undecyloxy)benzyl)isoindoline-1,3-dione (17). To a DCM solution (10 mL) of compound 6f (0.40 g, 0.82 mmol, 1.0 equiv) was added 1 drop of DMF, followed by the dropwise addition of SOCl₂ (0.15 g, 1.22 mmol, 1.5 equiv). The reaction mixture was stirred at 23° C. for 0.5 h. Then DCM and excess SOCl₂ was removed under reduced pressure. The obtained intermediate product was dissolved in dry DMF (10 mL) and potassium phthalimide (0.18 g, 0.98 mmol, 1.2 equiv) was added. The reaction mixture was stirred at 80° C. under N₂ atmosphere for 1 h. The mixture was cooled to 23° C. and DMF was removed under reduced pressure. Then water (20 mL) was added, and the mixture was extracted by DCM (20 mL) for three times. The organic phase was combined, dried over anhydrous MgSO₄ and filtered. The filtrate was concentrated and purified by column chromatography (SiO₂) with hexane/EtOAc=20/1 as the mobile phase to give the title compound as a light-yellow oil (0.40 g, 80%).

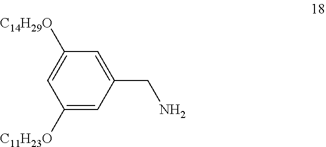

(3-(Tetradecyloxy)-5-(undecyloxy)phenyl)methanamine (18). Compound 17 (0.38 g, 0.61 mmol, 1 equiv) and hydrazine monohydrate (NH₂NH₂H₂O, 0.31 g, 6.10 mmol, 10 equiv) were dissolved in EtOH (10 mL). The reaction mixture was heated at reflux for 2 h. The mixture was cooled to 23° C. and the solvent was removed under reduced pressure. Then water (10 mL) was added, and the mixture was extracted by DCM (10 mL) for three times. The organic phase was combined, dried over anhydrous MgSO₄ and filtered. Filtration and evaporation of the solvent yielded the title compound as a colorless oil (0.28 g, 95%).

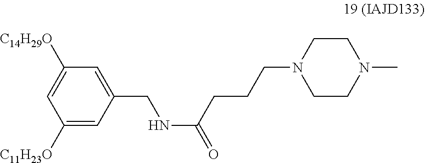

4-(4-Methylpiperazin-1-yl)-N-(3-(tetradecyloxy)-5-(undecyloxy)benzyl)butanamide (19, IAJD133). The synthesis and characterizations of 4-(4-methylpiperazin-1-yl)butanoic acid hydrochloride were available in the literature.⁴ Compound 18 (0.27 g, 0.55 mmol, 1 equiv) and 4-(4-methylpiperazin-1-yl)butanoic acid hydrochloride (0.12 g, 0.55 mmol, 1 equiv) were dissolved in 6 mL dry DCM. NEt₃ (3 drops) was added. DCC (0.23 g, 1.10 mmol, 2 equiv) was added in one portion into the above mixture. The reaction was allowed to stir at 23° C. for 12 h. Afterwards, the resulted white precipitates (urea) were removed by filtration and washed with DCM carefully. The filtrate was concentrated and purified by column chromatography (SiO₂) with DCM/MeOH=30/1, 10/1, 8/1 and 8/1 with 0.1% NEt₃ as the mobile phase. Then the product was dissolved in DCM (20 mL), which was washed by NaHCO₃solution (2%, 20 mL). The aqueous phase was extracted by DCM (20 mL) for another two times. The organic phase was combined and dried over anhydrous MgSO₄. Filtration and evaporation of the solvent yielded the title compound as a white solid (0.31 g, 86%).

Synthesis of Pentadecane-Based IAJDs with Nonsymmetric Alkyl Chains was performed according to Scheme 11 shown below:

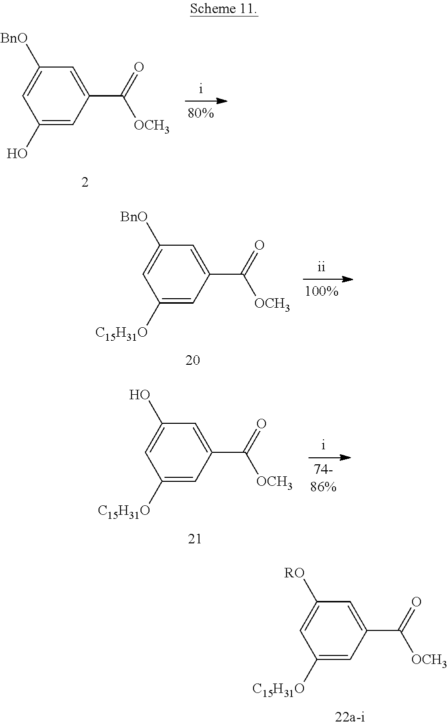

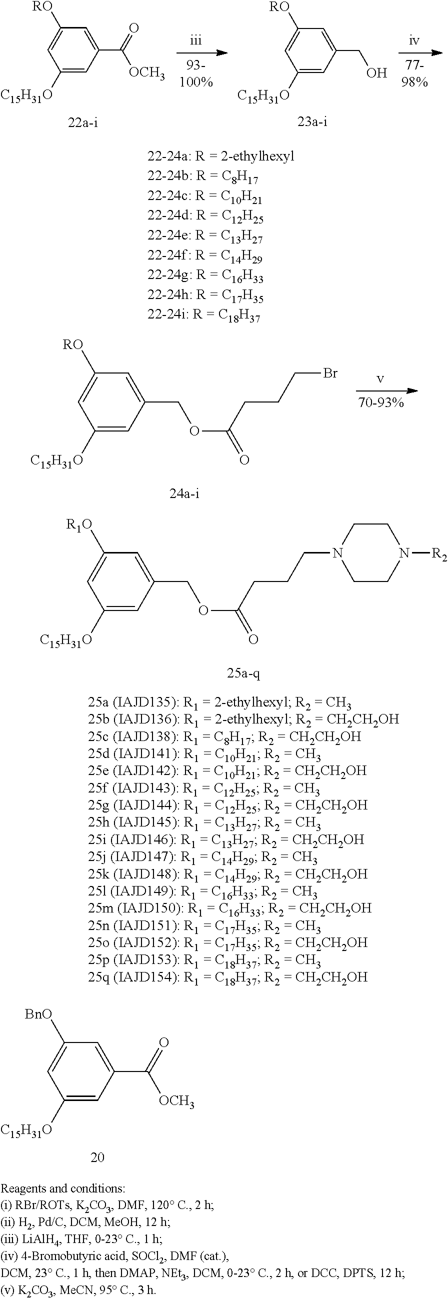

Methyl 3-(benzyloxy)-5-(pentadecyloxy)benzoate (20). Compound 2 (5.00 g, 19.36 mmol, 1 equiv), 1-bromopentadecane (6.20 g, 21.30 mmol, 1.1 equiv) and K₂CO₃ (5.35 g, 38.72 mmol, 2 equiv) were stirred in DMF (40 mL). The mixture was stirred at 120° C. under N₂ atmosphere for 2 h. After cooled to 23° C., the reaction mixture was poured into ice/water (150 mL) and the resulted white precipitates were filtered and collected. Then the precipitates were purified by recrystallization from minimum acetone to give the title compound as a white solid (7.20 g, 80%).

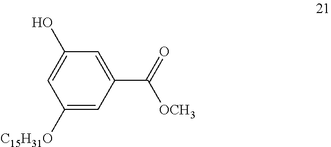

Methyl 3-hydroxy-5-(pentadecyloxy)benzoate (21). Compound 20 (7.20 g, 15.36 mmol) was dissolved in a mixture of DCM (40 mL) and methanol (20 mL). Then Pd/C (0.36 g, 5 wt %) was added and the flask was evacuated and filled with hydrogen for three times. The mixture was stirred at 23° C. for 12 h under hydrogen atmosphere. The reaction mixture was filtered through Celite, and the filter cake was washed with DCM carefully. Evaporation of the solvent yielded the title compound as a white solid (5.81 g, 100%).

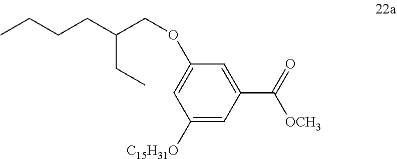

Methyl 3-((2-ethylhexyl)oxy)-5-(pentadecyloxy)benzoate (22a). Compound 21 (0.50 g, 1.32 mmol, 1 equiv), 3-(bromomethyl)heptane (0.27 g, 1.39 mmol, 1.05 equiv) and K₂CO₃ (0.37 g, 2.64 mmol, 2 equiv) were stirred in DMF (15 mL). The mixture was stirred at 80° C. under N₂ atmosphere for 12 h. The reaction mixture was cooled to 23° C. and DMF was removed under reduced pressure. Then water (20 mL) was added, and the mixture was extracted with DCM (20 mL) for three times. The organic phases were combined, dried over anhydrous MgSO₄ and filtered. The filtrate was concentrated and purified by column chromatography (SiO₂) with hexane/EtOAc=20/1 as the mobile phase to give the title compound as a light-yellow oil (0.48 g, 74%).

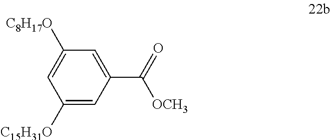

Methyl 3-(octyloxy)-5-(pentadecyloxy)benzoate (22b). Compound 21 (0.50 g, 1.32 mmol, 1 equiv), 1-bromooctane (0.27 g, 1.39 mmol, 1.05 equiv) and K₂CO₃ (0.37 g, 2.64 mmol, 2 equiv) were stirred in DMF (15 mL). The mixture was stirred at 120° C. under N₂ atmosphere for 2 h. After cooled to 23° C., the reaction mixture was poured into ice/water (100 mL) and the resulted white precipitates were filtered and collected. Then the precipitates were purified by recrystallization from minimum_acetone to_give_the title compound as a white solid (0.48 g, 84%).

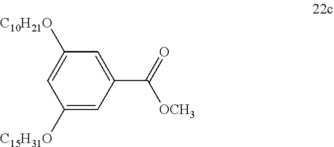

Methyl 3-(decyloxy)-5-(pentadecyloxy)benzoate (22c). Compound 22c was synthesized from compound 21 (0.50 g, 1.32 mmol, 1 equiv), 1-bromodecane (0.31 g, 1.39 mmol, 1.05 equiv) and K₂CO₃ (0.37 g, 2.64 mmol, 2 equiv) following a procedure similar to that used for the synthesis of compound 22b. The title compound was obtained as a white solid (0.58 g, 86%).

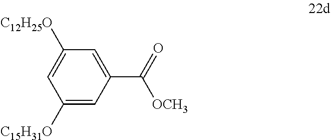

Methyl 3-(dodecyloxy)-5-(pentadecyloxy)benzoate (22d). Compound 22d was synthesized from compound 21 (0.50 g, 1.32 mmol, 1 equiv), 1-bromododecane (0.35 g, 1.39 mmol, 1.05 equiv) and K₂CO₃ (0.37 g, 2.64 mmol, 2 equiv) following a procedure similar to that used for the synthesis of compound 22b. The title compound was obtained as a white solid (0.60 g, 83%).

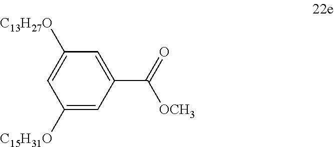

Methyl 3-(pentadecyloxy)-5-(tridecyloxy)benzoate (22e). Compound 22e was synthesized from compound 21 (0.50 g, 1.32 mmol, 1 equiv), C₁₃H₂₇OTs (12a, 0.51 g, 1.39 mmol, 1.05 equiv) and K₂CO₃ (0.37 g, 2.64 mmol, 2 equiv) following a procedure similar to that used for the synthesis of compound 22b. The title compound was obtained as a white solid (0.61 g, 82%).

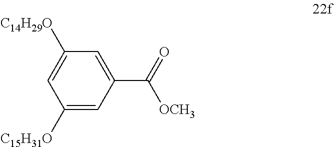

Methyl 3-(pentadecyloxy)-5-(tetradecyloxy)benzoate (22f). Compound 22f was synthesized from compound 21 (0.50 g, 1.32 mmol, 1 equiv), 1-bromotetradecane (0.39 g, 1.39 mmol, 1.05 equiv) and K₂CO₃ (0.37 g, 2.64 mmol, 2 equiv) following a procedure similar to that used for the synthesis of compound 22b. The title compound was obtained as a white solid (0.56 g, 74%).

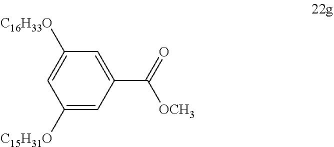

Methyl 3-(hexadecyloxy)-5-(pentadecyloxy)benzoate (22g). Compound 22g was synthesized from compound 21 (0.50 g, 1.32 mmol, 1 equiv), 1-bromohexadecane (0.43 g, 1.39 mmol, 1.05 equiv) and K₂CO₃ (0.37 g, 2.64 mmol, 2 equiv) following a procedure similar to that used for the synthesis of compound 22b. The title compound was obtained as a white solid (0.60 g, 75%).

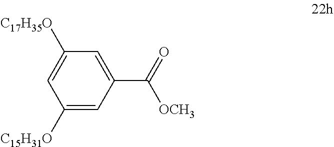

Methyl 3-(heptadecyloxy)-5-(pentadecyloxy)benzoate (22h). Compound 22h was synthesized from compound 21 (0.50 g, 1.32 mmol, 1 equiv), C₁₇H₃₅OTs (12b, 0.43 g, 1.39 mmol, 1.05 equiv) and K₂CO₃ (0.37 g, 2.64 mmol, 2 equiv) following a procedure similar to that used for the synthesis of compound 22b. The title compound was obtained as a white solid (0.67 g, 83%).

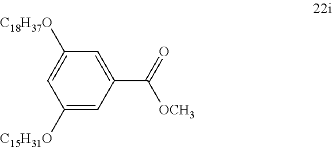

Methyl 3-(octadecyloxy)-5-(pentadecyloxy)benzoate (22i). Compound 22i was synthesized from compound 21 (0.50 g, 1.32 mmol, 1 equiv), 1-bromoctadecanene (0.46 g, 1.39 mmol, 1.05 equiv) and K₂CO₃ (0.37 g, 2.64 mmol, 2 equiv) following a procedure similar to that used for the synthesis of compound 22b. The title compound was obtained as a white solid (0.71 g, 85%).

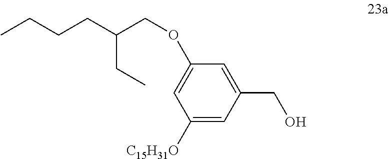

(3-((2-Ethylhexyl)oxy)-5-(pentadecyloxy)phenyl)methanol (23a). Compound 22a (0.32 g, 0.65 mmol, 1 equiv) was dissolved in 5 mL dry THF, which was added dropwise to a slurry of LiAlH₄ (25 mg, 0.65 mmol, 1 equiv) in dry THF (5 mL) at 0° C. under N₂ atmosphere. The resulted mixture was stirred at 23° C. for 1 h. The reaction was quenched by the successive addition of water (0.3 mL), 15% NaOH aqueous solution (0.3 mL) and water (1.5 mL). Then the white precipitates in the mixture were filtered out and the filtrate was dried over anhydrous MgSO₄. Filtration and evaporation of the solvent yielded the title compound as a colorless oil (0.29 g, 97%).

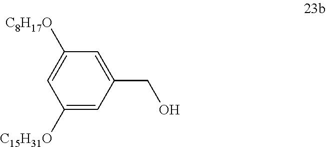

(3-(Octyloxy)-5-(pentadecyloxy)phenyl)methanol (23b). Compound 23b was synthesized from compound 22b (0.45 g, 0.91 mmol, 1 equiv) and LiAlH₄ (35 mg, 0.91 mmol, 1 equiv) following a procedure similar to that used for the synthesis of compound 23a. The title compound was obtained as a colorless oil (0.42 g, 100%).

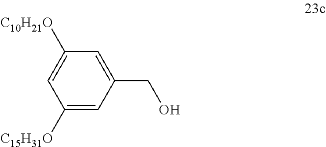

(3-(Decyloxy)-5-(pentadecyloxy)phenyl)methanol (23c). Compound 23c was synthesized from compound 22c (0.43 g, 0.83 mmol, 1 equiv) and LiAlH₄ (32 mg, 0.83 mmol, 1 equiv) following a procedure similar to that used for the synthesis of compound 23a. The title compound was obtained as a colorless oil (0.41 g, 100%).

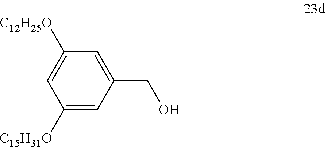

(3-(Dodecyloxy)-5-(pentadecyloxy)phenyl)methanol (23d). Compound 23d was synthesized from compound 22d (0.28 g, 0.51 mmol, 1 equiv) and LiAlH₄ (20 mg, 0.51 mmol, 1 equiv) following a procedure similar to that used for the synthesis of compound 23a. The title compound was obtained as a colorless oil (0.26 g, 100%).

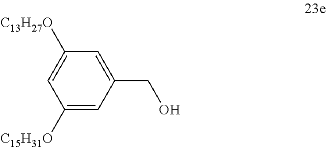

(3-(Pentadecyloxy)-5-(tridecyloxy)phenyl)methanol (23e). Compound 23e was synthesized from compound 22e (0.28 g, 0.49 mmol, 1 equiv) and LiAlH₄ (19 mg, 0.49 mmol, 1 equiv) following a procedure similar to that used for the synthesis of compound 23a. The title compound was obtained as a colorless oil (0.25 g, 96%).

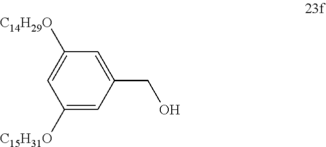

(3-(Pentadecyloxy)-5-(tetradecyloxy)phenyl)methanol (23f). Compound 23f was synthesized from compound 22f (0.53 g, 0.91 mmol, 1 equiv) and LiAlH₄ (35 mg, 0.91 mmol, 1 equiv) following a procedure similar to that used for the synthesis of compound 23a. The title compound was obtained as a colorless oil (0.50 g, 100%).

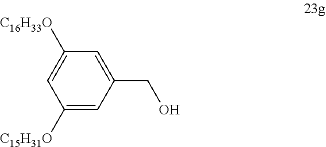

(3-(Hexadecyloxy)-5-(pentadecyloxy)phenyl)methanol (23g). Compound 23g was synthesized from compound 22g (0.50 g, 0.85 mmol, 1 equiv) and LiAlH₄ (32 mg, 0.85 mmol, 1 equiv) following a procedure similar to that used for the synthesis of compound 23a. The title compound was obtained as a white solid (0.44 g, 93%).

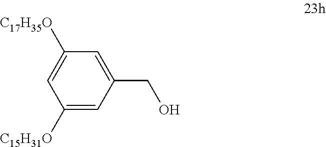

(3-(Heptadecyloxy)-5-(pentadecyloxy)phenyl)methanol (23h). Compound 23h was synthesized from compound 22h (0.55 g, 0.89 mmol, 1 equiv) and LiAlH₄ (34 mg, 0.89 mmol, 1 equiv) following a procedure similar to that used for the synthesis of compound 23a. The title compound was obtained as a white solid (0.49 g, 94%).

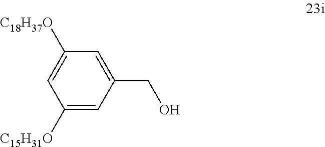

(3-(Octadecyloxy)-5-(pentadecyloxy)phenyl)methanol (23i). Compound 23i was synthesized from compound 22i (0.57 g, 0.90 mmol, 1 equiv) and LiAlH₄ (35 mg, 0.90 mmol, 1 equiv) following a procedure similar to that used for the synthesis of compound 23a. The title compound was obtained as a white solid (0.55 g, 100%).

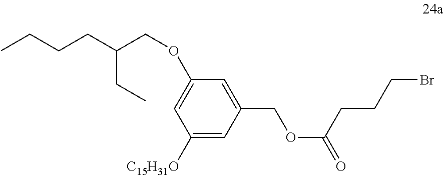

3-((2-Ethylhexyl)oxy)-5-(pentadecyloxy)benzyl 4-bromobutanoate (24a). Compound 23a (0.25 g, 0.54 mmol, 1 equiv), 4-bromobutyric acid (99 mg, 0.59 mmol, 1.1 equiv) and DPTS (0.17 g, 0.59 mmol, 1.1 equiv) were dissolved in 6 mL DCM. DCC (0.22 g, 1.08 mmol, 2 equiv) was added in one portion into the above mixture. The reaction mixture was allowed to stir at 23° C. for 12 h. The reaction mixture was filtered to remove the urea, which was washed with DCM carefully. The filtrate was concentrated and purified by column chromatography (SiO₂) with hexane/DCM=1/1 as the mobile phase to give the title compound as a colorless oil (0.31 g, 94%).

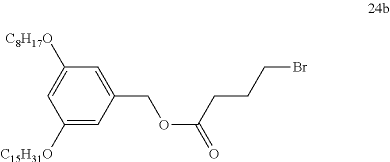

3-(Octyloxy)-5-(pentadecyloxy)benzyl 4-bromobutanoate (24b). Compound 24b was synthesized from compound 23b (0.40 g, 0.91 mmol, 1 equiv), 4-bromobutyric acid (0.17 g, 1.00 mmol, 1.1 equiv), DPTS (0.29 g, 1.00 mmol, 1.1 equiv) and DCC (0.38 g, 1.82 mmol, 2 equiv) following a procedure similar to that used for the synthesis of compound 24a. The title compound was obtained as a colorless oil (0.53 g, 95%).

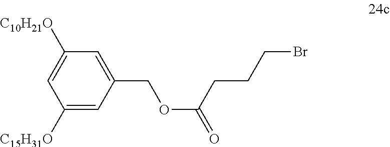

3-(Decyloxy)-5-(pentadecyloxy)benzyl 4-bromobutanoate (24c). Compound 24c was synthesized from compound 23c (0.40 g, 0.81 mmol, 1 equiv), 4-bromobutyric acid (0.15 g, 0.89 mmol, 1.1 equiv), DPTS (0.26 g, 0.89 mmol, 1.1 equiv) and DCC (0.33 g, 1.62 mmol, 2 equiv) following a procedure similar to that used for the synthesis of compound 24a. The title compound was obtained as a colorless oil (0.43 g, 83%).

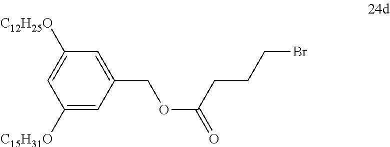

3-(Dodecyloxy)-5-(pentadecyloxy)benzyl 4-bromobutanoate (24d). Compound 24d was synthesized from compound 23d (0.26 g, 0.51 mmol, 1 equiv), 4-bromobutyric acid (94 mg, 0.56 mmol, 1.1 equiv), DPTS (0.16 g, 0.56 mmol, 1.1 equiv) and DCC (0.21 g, 1.02 mmol, 2 equiv) following a procedure similar to that used for the synthesis of compound 24a. The title compound was obtained as a colorless oil (0.30 g, 88%).

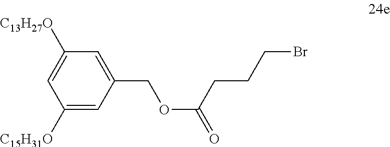

3-(Pentadecyloxy)-5-(tridecyloxy)benzyl 4-bromobutanoate (24e). Compound 24e was synthesized from compound 23e (0.23 g, 0.43 mmol, 1 equiv), 4-bromobutyric acid (79 mg, 0.47 mmol, 1.1 equiv), DPTS (0.14 g, 0.47 mmol, 1.1 equiv) and DCC (0.18 g, 0.86 mmol, 2 equiv) following a procedure similar to that used for the synthesis of compound 24a. The title compound was obtained as a colorless oil (0.29 g, 98%).

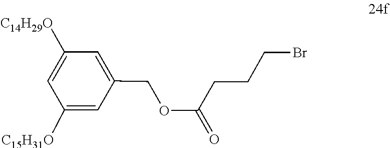

3-(Pentadecyloxy)-5-(tetradecyloxy)benzyl 4-bromobutanoate (24f). Compound 24f was synthesized from compound 23f (0.50 g, 0.91 mmol, 1 equiv), 4-bromobutyric acid (0.17 g, 1.00 mmol, 1.1 equiv), DPTS (0.29 g, 1.00 mmol, 1.1 equiv) and DCC (0.38 g, 1.82 mmol, 2 equiv) following a procedure similar to that used for the synthesis of compound 24a. The title compound was obtained as a colorless oil (0.60 g, 95%).

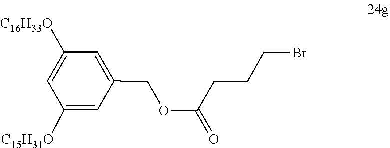

3-(Hexadecyloxy)-5-(pentadecyloxy)benzyl 4-bromobutanoate (24 g). Compound 24g was synthesized from compound 23g (0.42 g, 0.73 mmol, 1 equiv), 4-bromobutyric acid (0.13 g, 0.80 mmol, 1.1 equiv), DPTS (0.24 g, 0.80 mmol, 1.1 equiv) and DCC (0.30 g, 1.46 mmol, 2 equiv) following a procedure similar to that used for the synthesis of compound 24a. The title compound was obtained as a colorless oil (0.50 g, 96%).

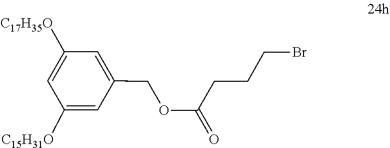

3-(Heptadecyloxy)-5-(pentadecyloxy)benzyl 4-bromobutanoate (24h). 4-Bromobutyric acid (0.15 g, 0.87 mmol, 1.1 equiv) was dissolved in 6 mL anhydrous DCM and 1 drop of DMF was added. SOCl₂ (0.21 g, 1.74 mmol, 2.2 equiv) was added and the mixture was stirred at 23° C. for 1 h. Afterwards, DCM and excess SOCl₂ was removed under reduced pressure to give the 4-bromobutanoyl chloride. Compound 23h (0.45 g, 0.76 mmol, 1.0 equiv) and NEt₃ (97 mg, 0.96 5 mmol, 1.3 equiv) were dissolved in 3 mL anhydrous DCM. To this mixture was added the DCM solution (3 mL) of above 4-bromobutanoyl chloride dropwise at 0° C. The mixture was allowed to warm to 23° C. with stirring during a 2 h period. Afterwards, water (30 mL) was added, and the mixture was extracted by DCM (30 mL) for three times. The organic phase was combined, dried over anhydrous MgSO₄ and filtered. The filtrate was concentrated and purified by column chromatography (SiO₂) with hexane/EtOAc=30/1 as the mobile phase to give the title compound as a colorless oil (0.43 g, 77%).

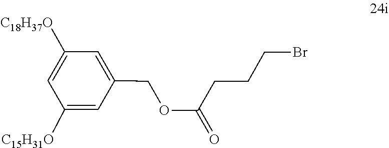

3-(Octadecyloxy)-5-(pentadecyloxy)benzyl 4-bromobutanoate (24i). Compound 24i was synthesized from compound 23i (0.56 g, 0.93 mmol, 1.0 equiv), 4-bromobutyric acid (0.17 g, 1.02 mmol, 1.1 equiv) and NEt₃ (0.12 g, 1.21 mmol, 1.3 equiv) following a procedure similar to that used for the synthesis of compound 24h. The title compound was obtained as a light-yellow oil (0.61 g, 88%).

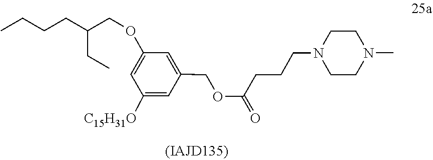

3-((2-Ethylhexyl)oxy)-5-(pentadecyloxy)benzyl 4-(4-methylpiperazin-1-yl)butanoate (25a, IAJD135). Compound 24a (0.15 g, 0.25 mmol, 1.0 equiv), 1-methylpiperazine (28 mg, 0.28 mmol, 1.1 equiv), K₂CO₃ (39 mg, 0.28 mmol, 1.1 equiv) were stirred in MeCN (15 mL). The mixture was heated at 95° C. for 3 h. The reaction mixture was cooled to 23° C. and the solvent was removed under reduced pressure. Then water (20 mL) was added, and the resulted mixture was extracted by DCM (20 mL) for three times. The organic phase was combined, dried over anhydrous MgSO₄, filtered and dried to give the crude product. The crude product was purified by column chromatography (SiO₂) with DCM/MeOH=30/1 and 15/1 as the mobile phase. Then the obtained product was dissolved in DCM (20 mL), which was washed by NaHCO₃solution (2%, 20 mL). The aqueous phase was extracted by DCM (20 mL) for another two times. The organic phase was collected and dried over anhydrous MgSO₄. Filtration and evaporation of the solvent yielded the title compound as a light-yellow oil (0.13 g, 82%).

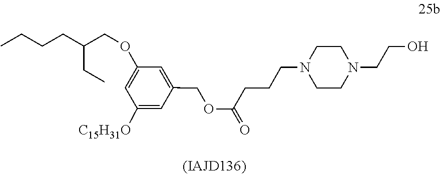

3-((2-Ethylhexyl)oxy)-5-(pentadecyloxy)benzyl 4-(4-(2-hydroxyethyl)piperazin-1-yl)butanoate (25b, IAJD136). Compound 25b was synthesized from compound 24a (0.16 g, 0.26 mmol), 1-(2-hydroxyethyl)piperazine (37 mg, 0.28 mmol) and K₂CO₃ (53 mg, 0.39 mmol) following a procedure similar to that used for the synthesis of compound 25a. The title compound was obtained as a colorless oil (0.13 g, 76%).

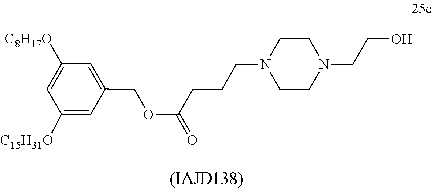

3-(Octyloxy)-5-(pentadecyloxy)benzyl 4-(4-(2-hydroxyethyl)piperazin-1-yl)butanoate (25c, IAJD138). Compound 25c was synthesized from compound 24b (0.24 g, 0.41 mmol), 1-(2-hydroxyethyl)piperazine (64 mg, 0.49 mmol) and K₂CO₃ (68 mg, 0.49 mmol) following a procedure similar to that used for the synthesis of compound 25a. The title compound was obtained as a light-yellow oil (0.25 g, 93%).

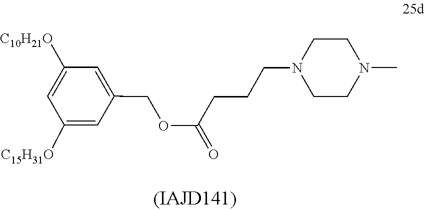

3-(Decyloxy)-5-(pentadecyloxy)benzyl 4-(4-methylpiperazin-1-yl)butanoate (25d, IAJD141). Compound 25d was synthesized from compound 24c (0.19 g, 0.30 mmol), 1-methylpiperazine (32 mg, 0.32 mmol) and K₂CO₃ (44 mg, 0.32 mmol) following a procedure similar to that used for the synthesis of compound 25a. The title compound was obtained as a colorless oil (0.17 g, 90%).

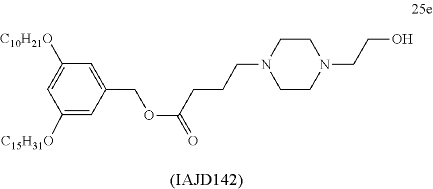

3-(Decyloxy)-5-(pentadecyloxy)benzyl 4-(4-(2-hydroxyethyl)piperazin-1-yl)butanoate (25e, IAJD142). Compound 25e was synthesized from compound 24c (0.19 g, 0.30 mmol), 1-(2-hydroxyethyl)piperazine (42 mg, 0.32 mmol) and K₂CO₃ (44 mg, 0.32 mmol) following a procedure similar to that used for the synthesis of compound 25a. The title compound was obtained as a light-yellow oil (0.18 g, 90%).

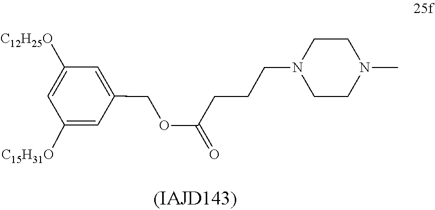

3-(Dodecyloxy)-5-(pentadecyloxy)benzyl 4-(4-methylpiperazin-1-yl)butanoate (25f, IAJD143). Compound 25f was synthesized from compound 24d (0.15 g, 0.22 mmol), 1-methylpiperazine (23 mg, 0.23 mmol) and K₂CO₃ (32 mg, 0.23 mmol) following a procedure similar to that used for the synthesis of compound 25a. The title compound was obtained as a colorless oil (0.13 g, 87%).

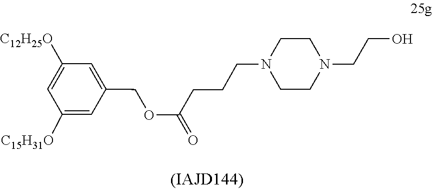

3-(Dodecyloxy)-5-(pentadecyloxy)benzyl 4-(4-(2-hydroxyethyl)piperazin-1-yl)butanoate (25g, IAJD144). Compound 25g was synthesized from compound 24d (0.15 g, 0.22 mmol), 1-(2-hydroxyethyl)piperazine (30 mg, 0.23 mmol) and K₂CO₃ (32 mg, 0.23 mmol) following a procedure similar to that used for the synthesis of compound 25a. The title compound was obtained as a colorless oil (0.14 g, 88%).

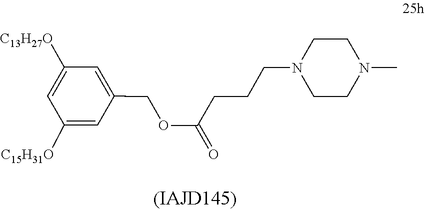

3-(pentadecyloxy)-5-(tridecyloxy)benzyl 4-(4-methylpiperazin-1-yl)butanoate (25h, IAJD145). Compound 25h was synthesized from compound 24e (0.15 g, 0.22 mmol), 1-methylpiperazine (23 mg, 0.23 mmol) and K₂CO₃ (32 mg, 0.23 mmol) following a procedure similar to that used for the synthesis of compound 25a. The title compound was obtained as a colorless oil (0.13 g, 87%).

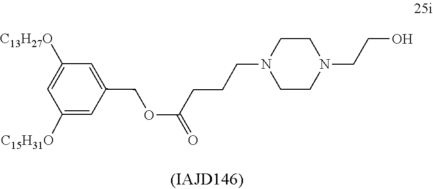

3-(Pentadecyloxy)-5-(tridecyloxy)benzyl 4-(4-(2-hydroxyethyl)piperazin-1-yl)butanoate (25i, IAJD146). Compound 25i was synthesized from compound 24e (0.15 g, 0.22 mmol), 1-(2-hydroxyethyl)piperazine (30 mg, 0.23 mmol) and K₂CO₃ (32 mg, 0.23 mmol) following a procedure similar to that used for the synthesis of compound 25a. The title compound was obtained as a light-yellow oil (0.14 g, 89%).

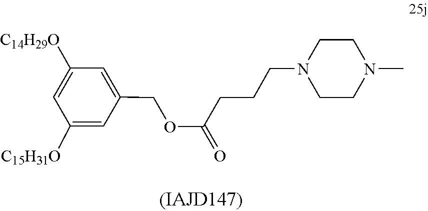

3-(Pentadecyloxy)-5-(tetradecyloxy)benzyl 4-(4-methylpiperazin-1-yl)butanoate (25j, IAJD147). Compound 25j was synthesized from compound 24f (0.28 g, 0.40 mmol), 1-methylpiperazine (44 mg, 0.44 mmol) and K₂CO₃ (67 mg, 0.48 mmol) following a procedure similar to that used for the synthesis of compound 25a. The title compound was obtained as a light-yellow oil (0.24 g, 88%).

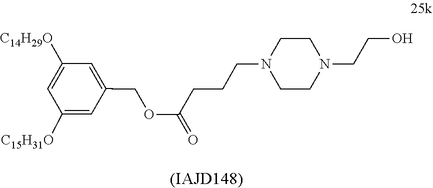

3-(Pentadecyloxy)-5-(tetradecyloxy)benzyl 4-(4-(2-hydroxyethyl)piperazin-1-yl)butanoate (25k, IAJD148). Compound 25k was synthesized from compound 24f (0.28 g, 0.40 mmol), 1-(2-hydroxyethyl)piperazine (57 mg, 0.44 mmol) and K₂CO₃ (67 mg, 0.48 mmol) following a procedure similar to that used for the synthesis of compound 25a. The title compound was obtained as a light-yellow oil (0.25 g, 92%).

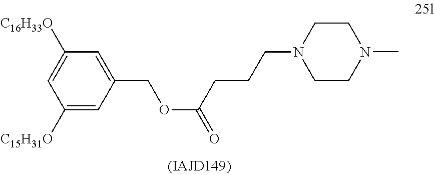

3-(Hexadecyloxy)-5-(pentadecyloxy)benzyl 4-(4-methylpiperazin-1-yl)butanoate (251, IAJD149). Compound 251 was synthesized from compound 24g (0.25 g, 0.35 mmol), 1-methylpiperazine (38 mg, 0.38 mmol) and K₂CO₃ (72 mg, 0.52 mmol) following a procedure similar to that used for the synthesis of compound 25a. The title compound was obtained as a colorless oil (0.21 g, 81%).

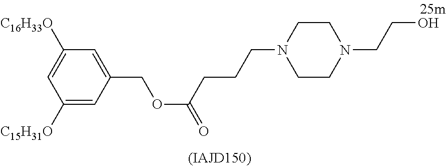

3-(Hexadecyloxy)-5-(pentadecyloxy)benzyl 4-(4-(2-hydroxyethyl)piperazin-1-yl)butanoate (25m, IAJD150). Compound 25m was synthesized from compound 24g (0.25 g, 0.35 mmol), 1-(2-hydroxyethyl)piperazine (50 mg, 0.38 mmol) and K₂CO₃ (72 mg, 0.52 mmol) following a procedure similar to that used for the synthesis of compound 25a. The title compound was obtained as a colorless oil (0.22 g, 81%).

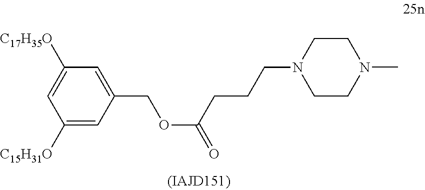

3-(Heptadecyloxy)-5-(pentadecyloxy)benzyl 4-(4-methylpiperazin-1-yl)butanoate (25n, IAJD151). Compound 25n was synthesized from compound 24h (0.16 g, 0.22 mmol), 1-methylpiperazine (23 mg, 0.23 mmol) and K₂CO₃ (32 mg, 0.23 mmol) following a procedure similar to that used for the synthesis of compound 25a. The title compound was obtained as a light-yellow oil (0.15 g, 88%).

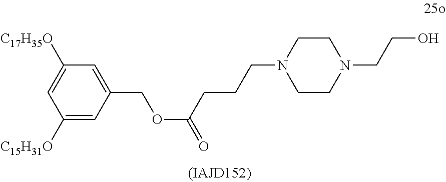

3-(Heptadecyloxy)-5-(pentadecyloxy)benzyl 4-(4-(2-hydroxyethyl)piperazin-1-yl)butanoate (250, IAJD152). Compound 25o was synthesized from compound 24h (0.15 g, 0.20 mmol), 1-(2-hydroxyethyl)piperazine (27 mg, 0.21 mmol) and K₂CO₃ (29 mg, 0.21 mmol) following a procedure similar to that used for the synthesis of compound 25a. The title compound was obtained as a colorless oil (0.14 g, 88%).

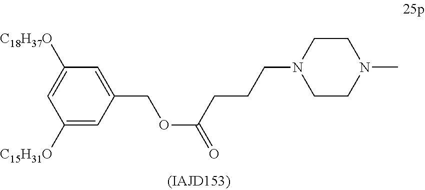

3-(Octadecyloxy)-5-(pentadecyloxy)benzyl 4-(4-methylpiperazin-1-yl)butanoate (25p, IAJD153). Compound 25p was synthesized from compound 24i (0.31 g, 0.41 mmol), 1-methylpiperazine (45 mg, 0.45 mmol) and K₂CO₃ (85 mg, 0.62 mmol) following a procedure similar to that used for the synthesis of compound 25a. The title compound was obtained as a colorless oil (0.21 g, 70%).

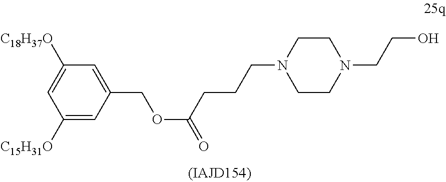

3-(Octadecyloxy)-5-(pentadecyloxy)benzyl 4-(4-(2-hydroxyethyl)piperazin-1-yl)butanoate (25q, IAJD154). Compound 25q was synthesized from compound 24i (0.31 g, 0.41 mmol), 1-(2-hydroxyethyl)piperazine (60 mg, 0.44 mmol) and K₂CO₃ (85 mg, 0.62 mmol) following a procedure similar to that used for the synthesis of compound 25a. The title compound was obtained as a colorless oil (0.25 g, 76%).

Synthesis of tridecane-based IAJDs with nonsymmetric alkyl chains was performed according to Scheme 12 shown below:

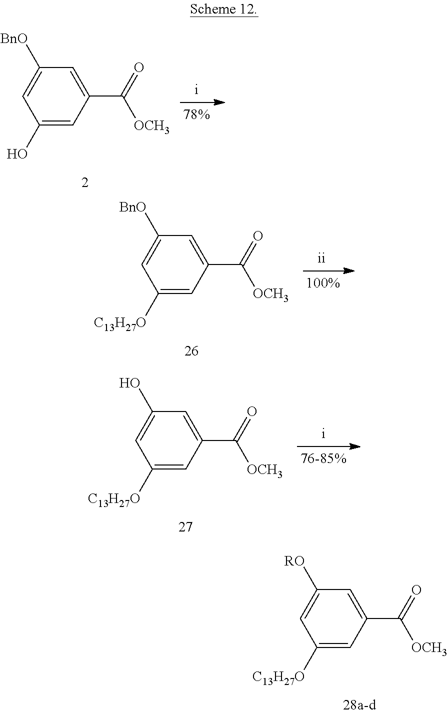

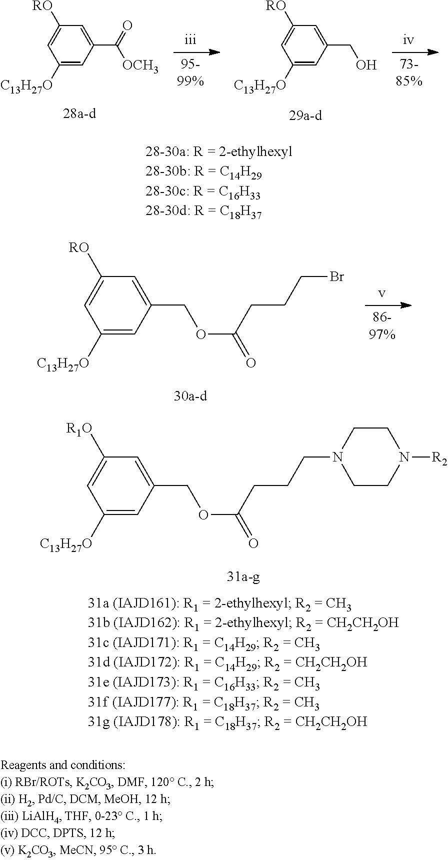

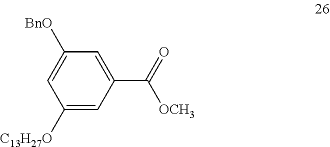

Methyl 3-(benzyloxy)-5-(tridecyloxy)benzoate (26). Compound 2 (5.50 g, 21.30 mmol, 1 equiv), C₁₃H₂₇OTs (12a, 8.30 g, 23.43 mmol, 1.1 equiv) and K₂CO₃ (5.89 g, 42.60 mmol, 2 equiv) were stirred in DMF (40 mL). The mixture was stirred at 120° C. under N₂ atmosphere for 2 h. After cooled to 23° C., the reaction mixture was poured into ice/water (150 mL) and the resulted white precipitates were filtered and collected. Then the precipitates were purified by recrystallization from minimum acetone to give the title compound as a white solid (7.30 g, 78%).

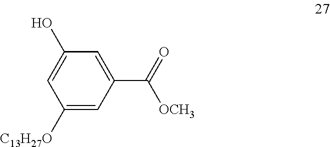

Methyl 3-hydroxy-5-(tridecyloxy)benzoate (27). Compound 26 (6.86 g, 15.57 mmol) was dissolved in a mixture of DCM (50 mL) and methanol (25 mL). Then Pd/C (0.41 g, 6 wt %) was added and the flask was evacuated and filled with hydrogen for three times. The mixture was stirred at 23° C. for 12 h under hydrogen atmosphere. The reaction mixture was filtered through Celite, and the filter cake was washed with DCM carefully. Evaporation of the solvent yielded the title compound as a white solid (5.46 g, 100%).

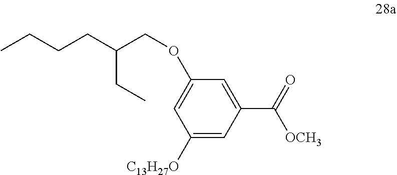

Methyl 3-((2-ethylhexyl)oxy)-5-(tridecyloxy)benzoate (28a). Compound 27 (0.60 g, 1.71 mmol, 1 equiv), 3-(bromomethyl)heptane (0.36 g, 1.88 mmol, 1.1 equiv) and K₂CO₃ (0.47 g, 3.42 mmol, 2 equiv) were stirred in DMF (15 mL). The mixture was stirred at 80° C. under N₂ atmosphere for 12 h. The reaction mixture was cooled to 23° C. and DMF was removed under reduced pressure. Then water (20 mL) was added, and the mixture was extracted with DCM (20 mL) for three times. The organic phases were combined, dried over anhydrous MgSO₄ and filtered. The filtrate was concentrated and purified by column chromatography (SiO₂) with hexane/EtOAc=20/1 as the mobile phase to give the title compound as a light-yellow oil (0.60 g, 76%).

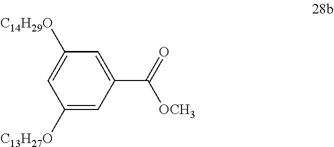

Methyl 3-(tetradecyloxy)-5-(tridecyloxy)benzoate (28b). Compound 27 (0.60 g, 1.71 mmol, 1 equiv), 1-bromotetradecane (0.52 g, 1.88 mmol, 1.1 equiv) and K₂CO₃ (0.47 g, 3.42 mmol, 2 equiv) were stirred in DMF (15 mL). The mixture was stirred at 120° C. under N₂ atmosphere for 2 h. After cooled to 23° C., the reaction mixture was poured into ice/water (100 mL) and the resulted white precipitates were filtered and collected. Then the precipitates were purified by recrystallization from minimum acetone to give the title compound as a white solid (0.76 g, 81%).

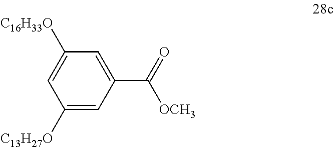

Methyl 3-(hexadecyloxy)-5-(tridecyloxy)benzoate (28c). Compound 28c was synthesized from compound 27 (0.60 g, 1.71 mmol, 1 equiv), 1-bromohexadecane (0.57 g, 1.88 mmol, 1.1 equiv) and K₂CO₃ (0.47 g, 3.42 mmol, 2 equiv) following a procedure similar to that used for the synthesis of compound 28b. The title compound was obtained as a white solid (0.84 g, 85%).

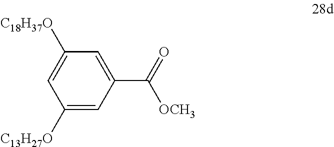

Methyl 3-(octadecyloxy)-5-(tridecyloxy)benzoate (28d). Compound 28d was synthesized from compound 27 (0.42 g, 1.20 mmol, 1 equiv), 1-bromoctadecanene (0.44 g, 1.32 mmol, 1.1 equiv) and K₂CO₃ (0.33 g, 2.40 mmol, 2 equiv) following a procedure similar to that used for the synthesis of compound 28b. The title compound was obtained as a white solid (0.56 g, 78%).

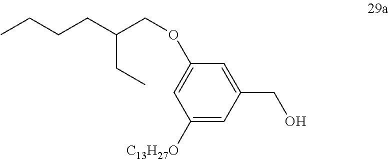

(3-((2-Ethylhexyl)oxy)-5-(tridecyloxy)phenyl)methanol (29a). Compound 28a (0.60 g, 1.30 mmol, 1 equiv) was dissolved in 5 mL dry THF, which was added dropwise to a slurry of LiAlH₄ (49 mg, 1.30 mmol, 1 equiv) in dry THF (5 mL) at 0° C. under N₂ atmosphere. The resulted mixture was stirred at 23° C. for 1 h. The reaction was quenched by the successive addition of water (0.3 mL), 15% NaOH aqueous solution (0.3 mL) and water (1.5 mL). Then the white precipitates in the mixture were filtered out and the filtrate was dried over anhydrous MgSO₄. Filtration and evaporation of the solvent yielded the title compound as a colorless oil (0.54 g, 95%).

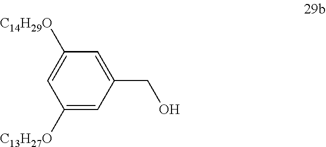

(3-(Tetradecyloxy)-5-(tridecyloxy)phenyl)methanol (29b). Compound 29b was synthesized from compound 28b (0.75 g, 1.37 mmol, 1 equiv) and LiAlH₄ (52 mg, 1.37 mmol, 1 equiv) following a procedure similar to that used for the synthesis of compound 29a. The title compound was obtained as a colorless oil (0.68 g, 96%).

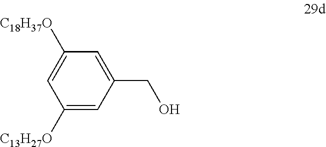

(3-(Hexadecyloxy)-5-(tridecyloxy)phenyl)methanol (29c). Compound 29c was synthesized from compound 28c (0.86 g, 1.50 mmol, 1 equiv) and LiAlH₄ (58 mg, 1.50 mmol, 1 equiv) following a procedure similar to that used for the synthesis of compound 29a. The title compound was obtained as a white solid (0.80 g, 98%).

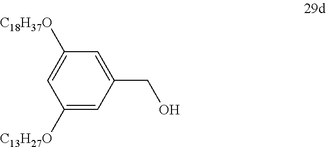

(3-(Octadecyloxy)-5-(tridecyloxy)phenyl)methanol (29d). Compound 29d was synthesized from compound 28d (0.53 g, 0.88 mmol, 1 equiv) and LiAlH₄ (34 mg, 0.88 mmol, 1 equiv) following a procedure similar to that used for the synthesis of compound 29a. The title compound was obtained as a white solid (0.50 g, 99%).

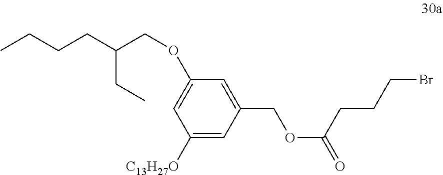

3-((2-Ethylhexyl)oxy)-5-(tridecyloxy)benzyl 4-bromobutanoate (30a). Compound 29a (0.46 g, 1.06 mmol, 1 equiv), 4-bromobutyric acid (0.20 g, 1.17 mmol, 1.1 equiv) and DPTS (0.34 g, 1.17 mmol, 1.1 equiv) were dissolved in 6 mL DCM. DCC (0.44 g, 2.12 mmol, 2 equiv) was added in one portion into the above mixture. The reaction mixture was allowed to stir at 23° C. for 12 h. The reaction mixture was filtered to remove the urea, which was washed with DCM carefully. The filtrate was concentrated and purified by column chromatography (SiO₂) with hexane/DCM=1/1 as the mobile phase to give the title compound as a colorless oil (0.46 g, 74%).

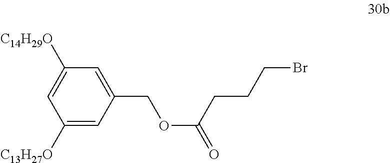

3-(Tetradecyloxy)-5-(tridecyloxy)benzyl 4-bromobutanoate (30b). Compound 30b was synthesized from compound 29b (0.30 g, 0.58 mmol, 1 equiv), 4-bromobutyric acid (0.11 g, 0.64 mmol, 1.1 equiv), DPTS (0.19 g, 0.64 mmol, 1.1 equiv) and DCC (0.24 g, 1.16 mmol, 2 equiv) following a procedure similar to that used for the synthesis of compound 30a. The title compound was obtained as a colorless oil (0.31 g, 79%).

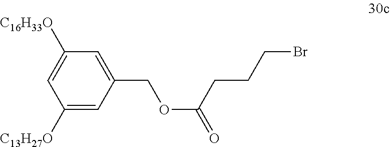

3-(Hexadecyloxy)-5-(tridecyloxy)benzyl 4-bromobutanoate (30c). Compound 30c was synthesized from compound 29c (0.30 g, 0.55 mmol, 1 equiv), 4-bromobutyric acid (0.10 g, 0.61 mmol, 1.1 equiv), DPTS (0.18 g, 0.61 mmol, 1.1 equiv) and DCC (0.23 g, 1.10 mmol, 2 equiv) following a procedure similar to that used for the synthesis of compound 30a. The title compound was obtained as a colorless oil (0.28 g, 73%).

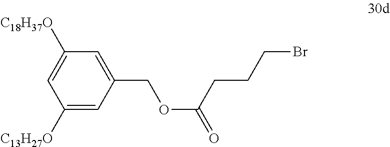

3-(Octadecyloxy)-5-(tridecyloxy)benzyl 4-bromobutanoate (30d). Compound 30d was synthesized from compound 29d (0.44 g, 0.77 mmol, 1 equiv), 4-bromobutyric acid (0.14 g, 0.85 mmol, 1.1 equiv), DPTS (0.25 g, 0.85 mmol, 1.1 equiv) and DCC (0.32 g, 1.54 mmol, 2 equiv) following a procedure similar to that used for the synthesis of compound 30a. The title compound was obtained as a colorless oil (0.47 g, 85%).

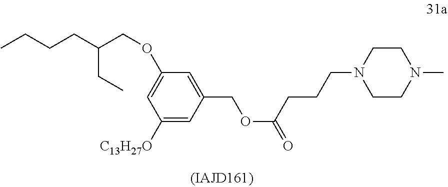

3-((2-Ethylhexyl)oxy)-5-(tridecyloxy)benzyl 4-(4-methylpiperazin-1-yl)butanoate (31a, IAJD161). Compound 30a (0.17 g, 0.29 mmol, 1.0 equiv), 1-methylpiperazine (30 mg, 0.30 mmol, 1.05 equiv), K₂CO₃ (42 mg, 0.30 mmol, 1.05 equiv) were stirred in MeCN (15 mL). The mixture was heated at 95° C. for 3 h. The reaction mixture was cooled to 23° C. and the solvent was removed under reduced pressure. Then water (20 mL) was added, and the resulted mixture was extracted by DCM (20 mL) for three times. The organic phase was combined, dried over anhydrous MgSO₄, filtered and dried to give the crude product. The crude product was purified by column chromatography (SiO₂) with DCM/MeOH=30/1 and 15/1 as the mobile phase. Then the obtained product was dissolved in DCM (20 mL), which was washed by NaHCO₃solution (2%, 20 mL). The aqueous phase was extracted by DCM (20 mL) for another two times. The organic phase was collected and dried over anhydrous MgSO₄. Filtration and evaporation of the solvent yielded the title compound as a colorless oil (0.15 g 88%).

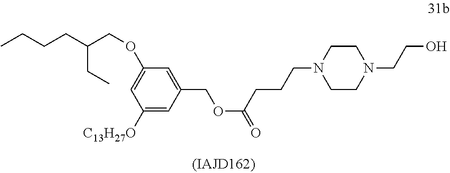

3-((2-Ethylhexyl)oxy)-5-(tridecyloxy)benzyl 4-(4-(2-hydroxyethyl)piperazin-1-yl)butanoate (31b, IAJD162). Compound 31b was synthesized from compound 30a (0.17 g, 0.29 mmol), 1-(2-hydroxyethyl)piperazine (39 mg, 0.30 mmol) and K₂CO₃ (42 mg, 0.30 mmol) following a procedure similar to that used for the synthesis of compound 31a. The title compound was obtained as a colorless oil (0.16 g, 89%).

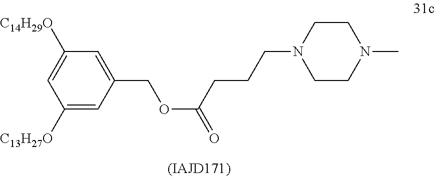

3-(Tetradecyloxy)-5-(tridecyloxy)benzyl 4-(4-methylpiperazin-1-yl)butanoate (31c, IAJD171). Compound 31c was synthesized from compound 30b (0.14 g, 0.21 mmol), 1-methylpiperazine (22 mg, 0.22 mmol) and K₂CO₃ (31 mg, 0.22 mmol) following a procedure similar to that used for the synthesis of compound 31a. The title compound was obtained as a colorless oil (0.12 g, 86%).

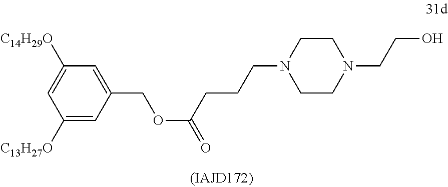

3-(Tetradecyloxy)-5-(tridecyloxy)benzyl 4-(4-(2-hydroxyethyl)piperazin-1-yl)butanoate (31d, IAJD172). Compound 31d was synthesized from compound 30b (0.14 g, 0.21 mmol), 1-(2-hydroxyethyl)piperazine (29 mg, 0.22 mmol) and K₂CO₃ (31 mg, 0.22 mmol) following a procedure similar to that used for the synthesis of compound 31a. The title compound was obtained as a colorless oil (0.13 g, 87%).

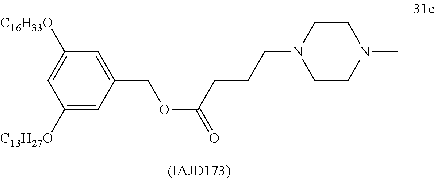

3-(Hexadecyloxy)-5-(tridecyloxy)benzyl 4-(4-methylpiperazin-1-yl)butanoate (31e, IAJD173). Compound 31e was synthesized from compound 30c (0.14 g, 0.20 mmol), 1-methylpiperazine (22 mg, 0.22 mmol) and K₂CO₃ (31 mg, 0.22 mmol) following a procedure similar to that used for the synthesis of compound 31a. The title compound was obtained as a light-yellow oil (0.12 g, 86%).

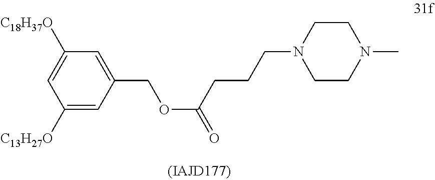

3-(Octadecyloxy)-5-(tridecyloxy)benzyl 4-(4-methylpiperazin-1-yl)butanoate (31f, IAJD177). Compound 31f was synthesized from compound 30d (0.20 g, 0.27 mmol), 1-methylpiperazine (30 mg, 0.30 mmol) and K₂CO₃ (41 mg, 0.30 mmol) following a procedure similar to that used for the synthesis of compound 31a. The title compound was obtained as a light-yellow oil (0.20 g, 97%).

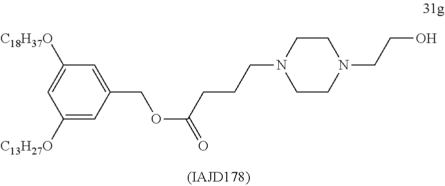

3-(Octadecyloxy)-5-(tridecyloxy)benzyl 4-(4-(2-hydroxyethyl)piperazin-1-yl)butanoate (31g, IAJD178). Compound 31g was synthesized from compound 30d (0.26 g, 0.36 mmol), 1-(2-hydroxyethyl)piperazine (48 mg, 0.37 mmol) and K₂CO₃ (51 mg, 0.37 mmol) following a procedure similar to that used for the synthesis of compound 31a. The title compound was obtained as a light-yellow oil (0.27 g, 96%).

Synthesis of Triethylene Glycol-Based IAJDs with Nonsymmetric Alkyl Chains was performed according to Scheme 13 shown below:

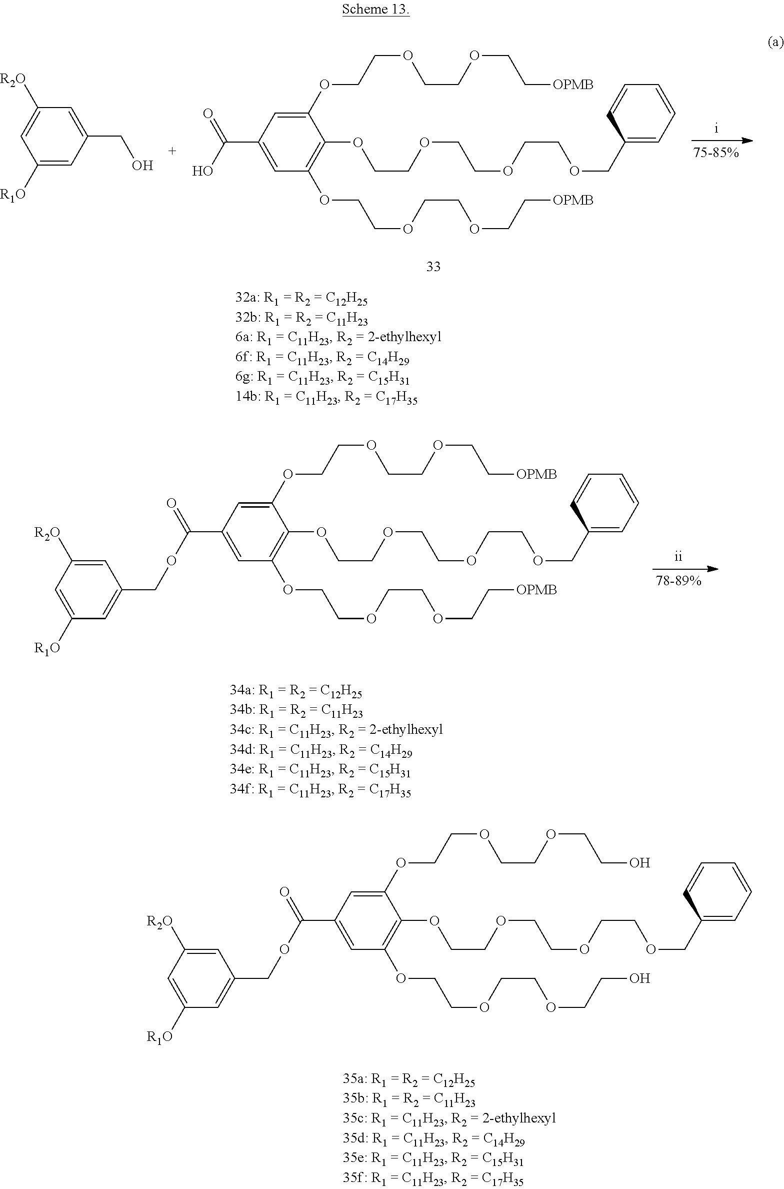

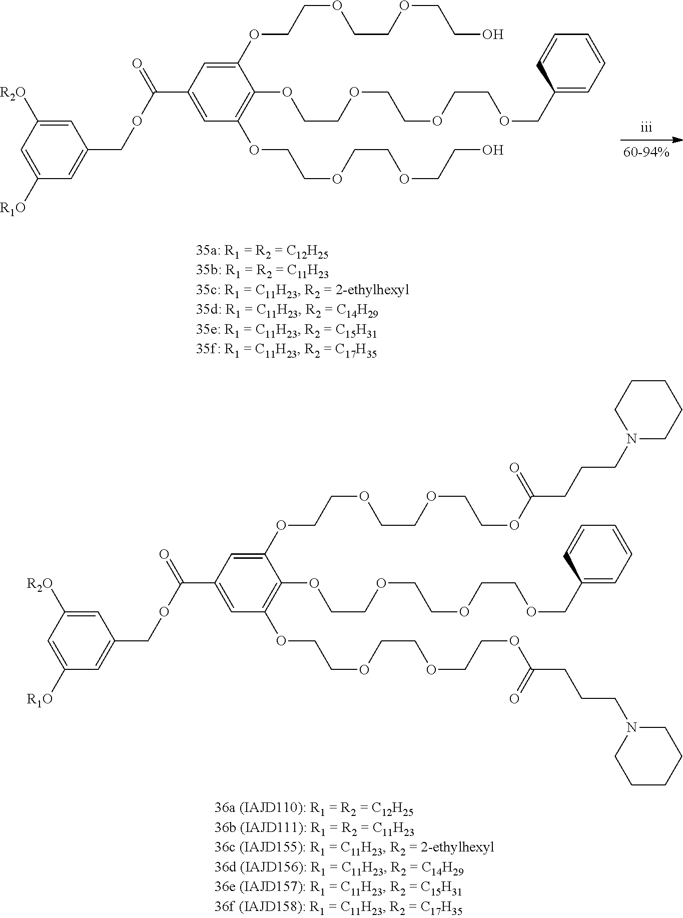

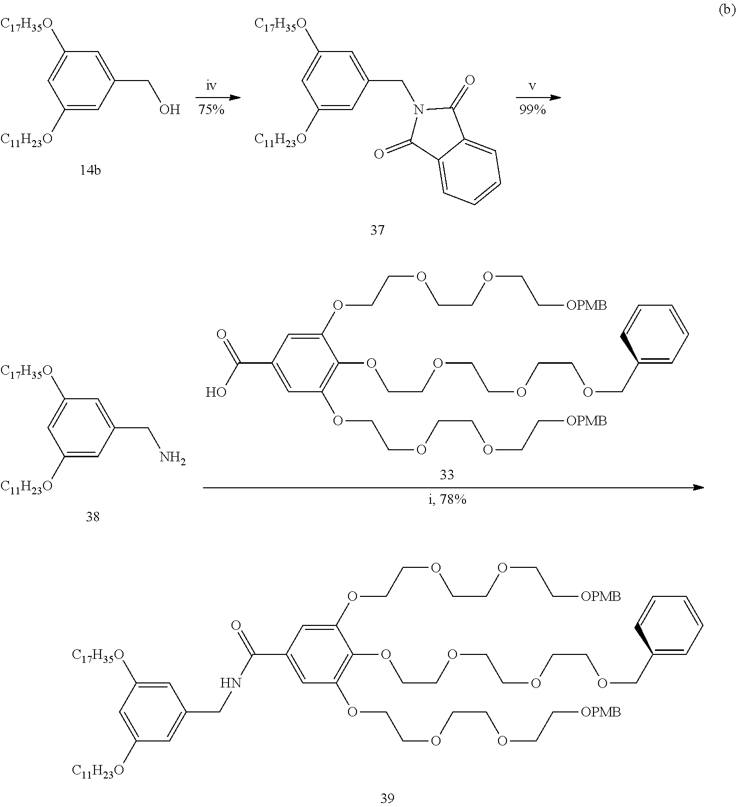

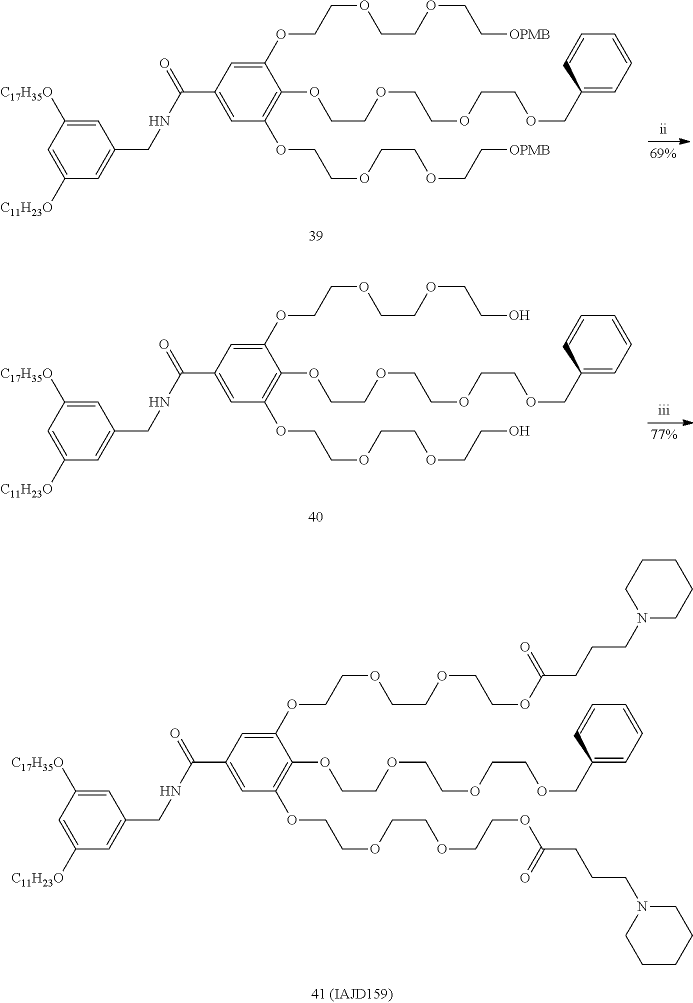

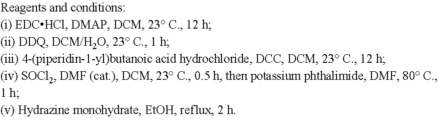

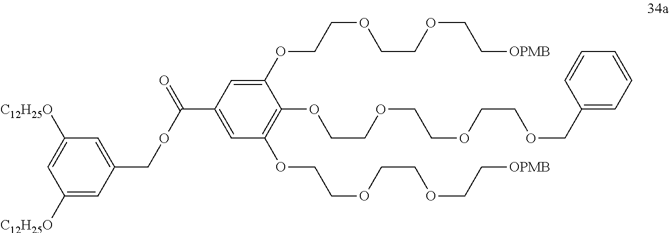

Compound 34a. Compound 32a was synthesized according to a previously reported procedure. Compound 33 was synthesized according to a procedure that was elaborated and optimized. Compound 32a (0.41 g, 0.86 mmol, 1.1 equiv), compound 33 (0.70 g, 0.78 mmol, 1.0 equiv), EDC·HCl (0.16 g, 0.86 mmol, 1.1 equiv) and DMAP (28 mg, 0.23 mmol, 0.3 equiv) were dissolved in dry DCM (8 mL). The reaction mixture was stirred at 23° C. for 12 h. Brine (30 mL) was added, and the mixture was extracted by DCM (30 mL) for three times. The organic phase was collected, dried over anhydrous MgSO₄ and filtered. The filtrate was concentrated and purified by column chromatography (SiO₂) with EtOAc/hexane=1/1 as the mobile phase to give the title compound as a colorless oil (0.83 g, 78%).

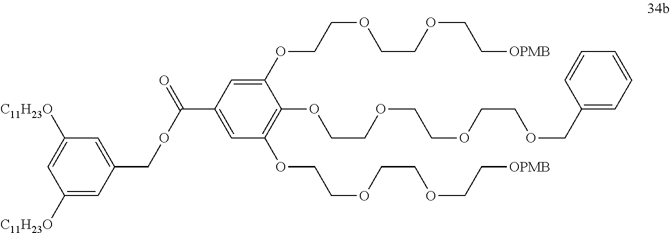

Compound 34b. Compound 32b was synthesized according to a previously reported procedure. Compound 34b was synthesized from compound 32b (0.39 g, 0.86 mmol) and compound 33 (0.70 g, 0.78 mmol) following a procedure similar to that used for the synthesis of compound 34a. The title compound was obtained as a colorless oil (0.78 g, 75%).

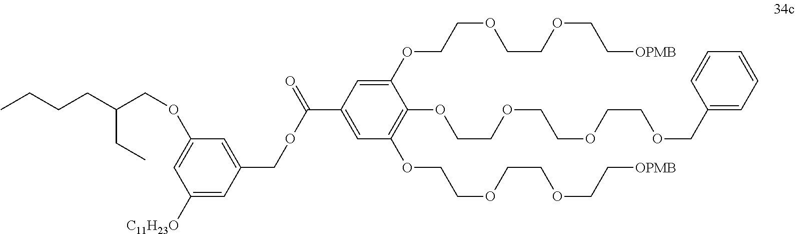

Compound 34c. Compound 34c was synthesized from compound 6a (0.10 g, 0.25 mmol) and compound 33 (0.22 g, 0.25 mmol) following a procedure similar to that used for the synthesis of compound 34a. The title compound was obtained as a colorless oil (0.26 g, 81%).

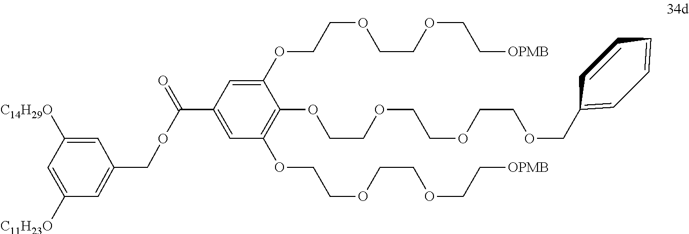

Compound 34d. Compound 34d was synthesized from compound 6f (0.12 g, 0.24 mmol) and compound 33 (0.22 g, 0.24 mmol) following a procedure similar to that used for the synthesis of compound 34a. The title compound was obtained as a colorless oil (0.28 g, 85%).

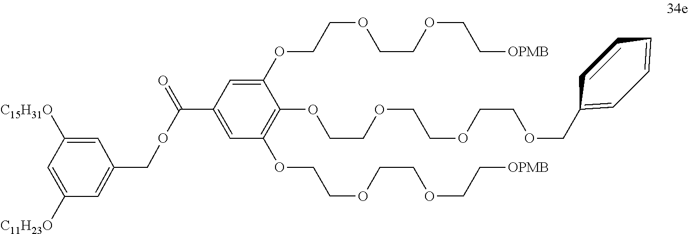

Compound 34e. Compound 34e was synthesized from compound 6g (0.12 g, 0.24 mmol) and compound 33 (0.22 g, 0.24 mmol) following a procedure similar to that used for the synthesis of compound 34a. The title compound was obtained as a colorless oil (0.27 g, 82%).

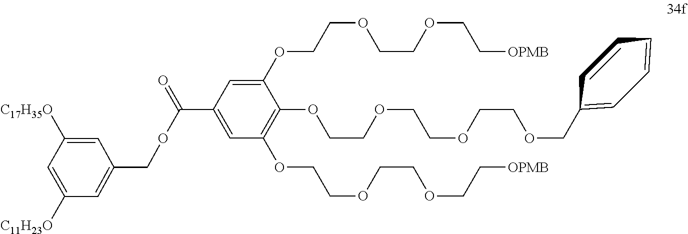

Compound 34f. Compound 34f was synthesized from compound 14b (0.17 g, 0.32 mmol) and compound 33 (0.29 g, 0.32 mmol) following a procedure similar to that used for the synthesis of compound 34a. The title compound was obtained as a colorless oil (0.35 g, 78%).

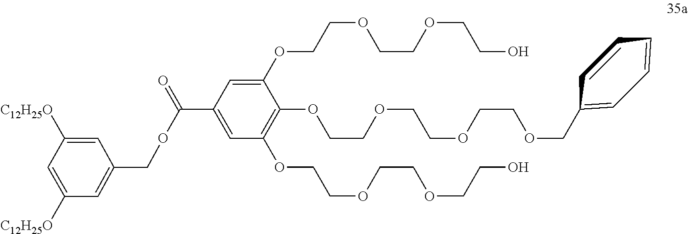

Compound 35a. Compound 34a (0.68 g, 0.50 mmol, 1 equiv) was dissolved in 6 mL DCM and 0.42 mL water (7 v %) was added. To this solution was added 2,3-dichloro-5,6-dicyano-1,4-benzoquinone (DDQ, 0.25 g, 1.10 mmol, 2.2 equiv). The reaction mixture was stirred at 23° C. for 1 h. Then the precipitates were filtered out and DCM (20 mL) was added. The mixture was washed by NaHCO₃aqueous solution (saturated), NaHSO₃ aqueous solution (2%) and NaHCO₃aqueous solution (saturated) successively. The organic phase was collected, dried over anhydrous MgSO₄ and filtered. The filtrate was concentrated and purified by column chromatography (SiO₂) with EtOAc/MeOH=20/1 as the mobile phase to give the title compound as a light-yellow oil (0.45 g, 80%).

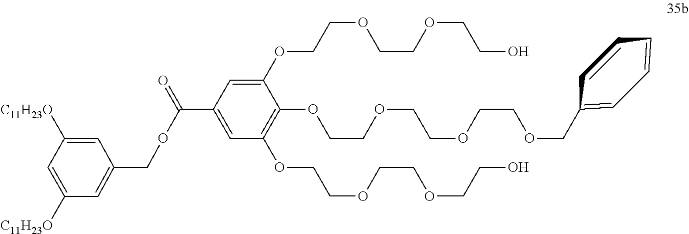

Compound 35b. Compound 35b was synthesized from compound 34b (0.66 g, 0.50 mmol) and DDQ (0.25 g, 1.10 mmol) following a procedure similar to that used for the synthesis of compound 35a. The title compound was obtained as a light-yellow oil (0.42 g, 78%).

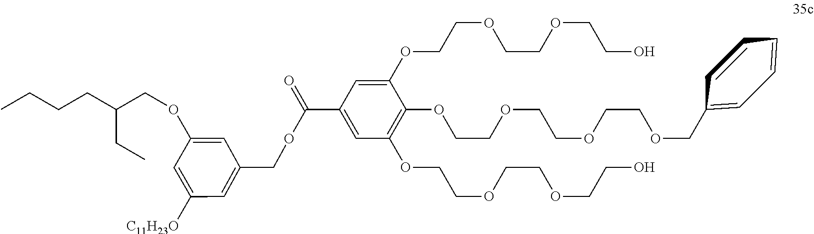

Compound 35c. Compound 35c was synthesized from compound 34c (0.25 g, 0.19 mmol) and DDQ (95 mg, 0.42 mmol) following a procedure similar to that used for the synthesis of compound 35a. The title compound was obtained as a light-yellow oil (0.17 g, 85%).

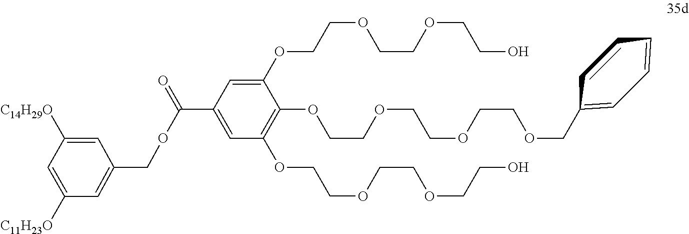

Compound 35d. Compound 35d was synthesized from compound 34d (0.26 g, 0.19 mmol) and DDQ (95 mg, 0.42 mmol) following a procedure similar to that used for the synthesis of compound 35a. The title compound was obtained as a light-yellow oil (0.17 g, 81%).

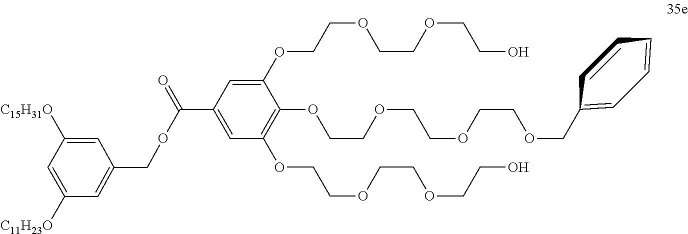

Compound 35e. Compound 35e was synthesized from compound 34e (0.25 g, 0.18 mmol) and DDQ (0.10 g, 0.44 mmol) following a procedure similar to that used for the synthesis of compound 35a. The title compound was obtained as a colorless oil (0.18 g, 89%).

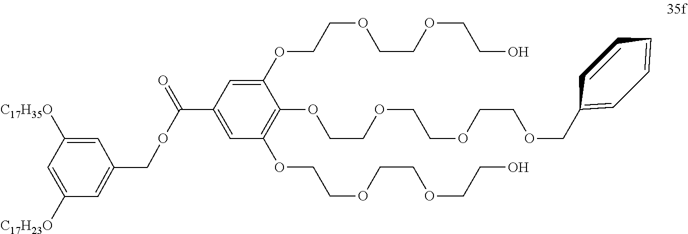

Compound 35f. Compound 35f was synthesized from compound 34f (0.34 g, 0.24 mmol) and DDQ (0.14 g, 0.61 mmol) following a procedure similar to that used for the synthesis of compound 35a. The title compound was obtained as a colorless oil (0.23 g, 81%).

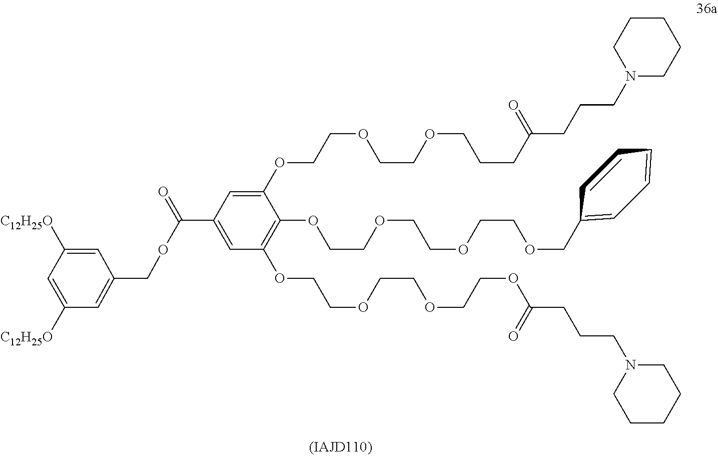

Compound 36a (IAJD110). Compound 35a (0.20 g, 0.18 mmol, 1 equiv) and 4-(piperidin-1-yl)butanoic acid hydrochloride (83 mg, 0.40 mmol, 2.2 equiv) were dissolved in 5 mL dry DCM. DCC (0.11 g, 0.54 mmol, 3 equiv) was added in one portion into the above mixture. The reaction mixture was stirred at 23° C. for 12 h. The mixture was filtered to remove the urea, which was washed with DCM carefully. The filtrate was concentrated and purified by column chromatography (SiO₂) with DCM/MeOH=30/1, 10/1, 8/1 and 8/1 with 0.1% NEt₃ as the mobile phase. Then the product was dissolved in DCM (20 mL), which was washed by NaHCO₃aqueous solution (2%, 20 mL). The aqueous phase was extracted by DCM (20 mL) for another two times. The organic phase was combined and dried over anhydrous MgSO₄. Filtration and evaporation of the solvent yielded the title compound as a light-yellow oil (0.23 g, 89%).

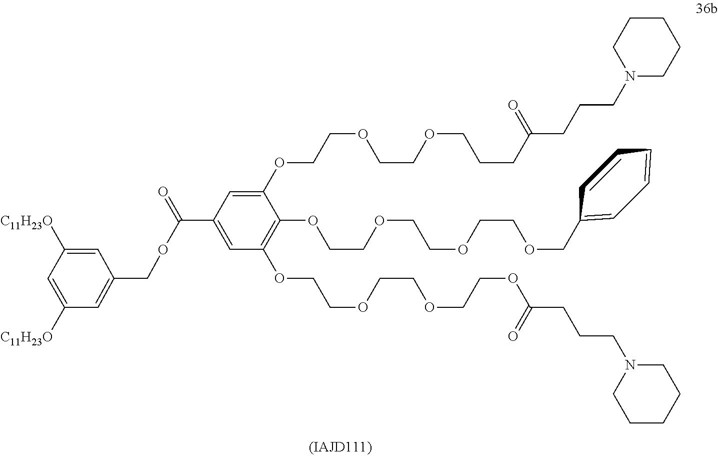

Compound 36b (IAJD111). Compound 36b was synthesized from compound 35b (0.20 g, 0.18 mmol), 4-(piperidin-1-yl)butanoic acid hydrochloride (83 mg, 0.40 mmol) and DCC (0.11 g, 0.54 mmol) following a procedure similar to that used for the synthesis of compound 36a. The title compound was obtained as a light-yellow oil (0.23 g, 92%).

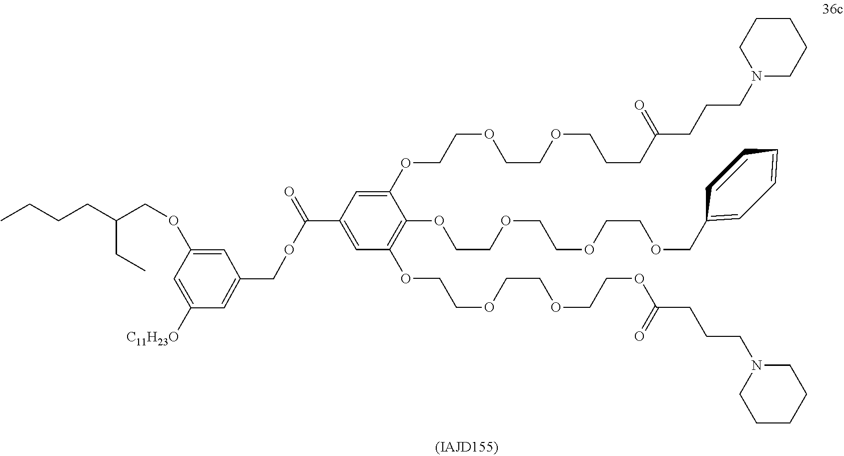

Compound 36c (IAJD155). Compound 36c was synthesized from compound 35c (0.12 g, 0.11 mmol), 4-(piperidin-1-yl)butanoic acid hydrochloride (50 mg, 0.24 mmol) and DCC (68 mg, 0.33 mmol) following a procedure similar to that used for the synthesis of compound 36a. The title compound was obtained as a light-yellow oil (0.13 g, 87%).

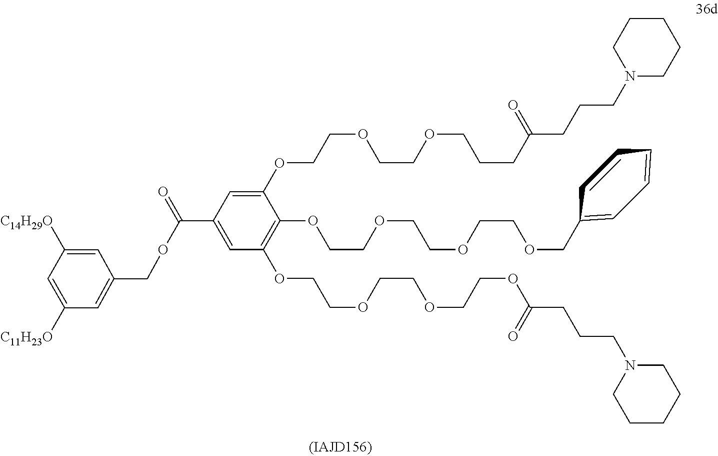

Compound 36d (IAJD156). Compound 36d was synthesized from compound 35d (0.14 g, 0.12 mmol), 4-(piperidin-1-yl)butanoic acid hydrochloride (54 mg, 0.26 mmol) and DCC (74 mg, 0.36 mmol) following a procedure similar to that used for the synthesis of compound 36a. The title compound was obtained as a light-yellow oil (0.14 g, 82%).

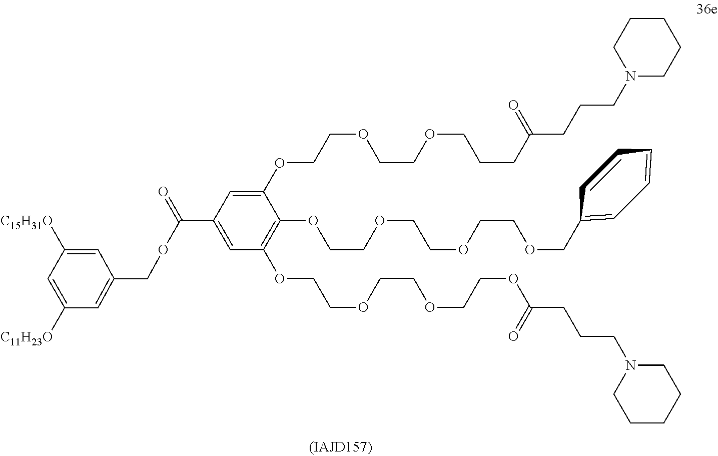

Compound 36e (IAJD157). Compound 36e was synthesized from compound 35e (0.18 g, 0.16 mmol), 4-(piperidin-1-yl)butanoic acid hydrochloride (84 mg, 0.40 mmol) and DCC (0.17 g, 0.80 mmol) following a procedure similar to that used for the synthesis of compound 36a. The title compound was obtained as a colorless oil (0.22 g, 94%).

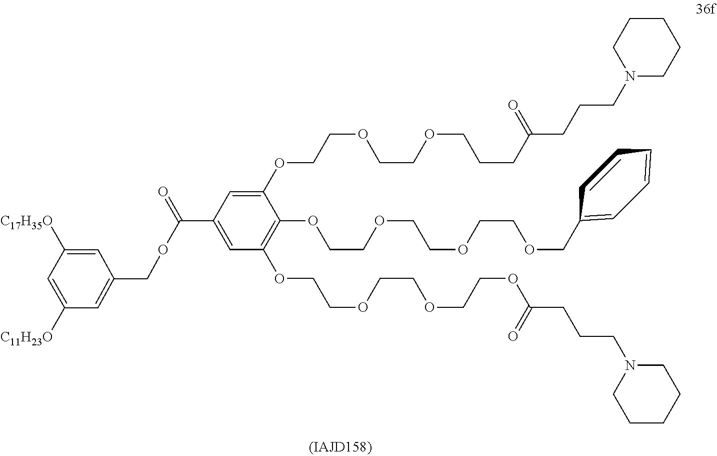

Compound 36f (IAJD158). Compound 36f was synthesized from compound 35f (0.23 g, 0.20 mmol), 4-(piperidin-1-yl)butanoic acid hydrochloride (0.10 g, 0.49 mmol) and DCC (0.20 g, 0.98 mmol) following a procedure similar to that used for the synthesis of compound 36a. The title compound was obtained as a colorless oil (0.17 g, 60%).

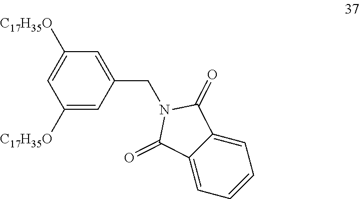

Compound 37. Compound 14b (0.35 g, 0.66 mmol, 1 equiv) was dissolved in dry DCM (10 mL) and 1 drop of DMF was added, followed by the dropwise addition of SOCl₂ (0.18 g, 0.99 mmol, 1.5 equiv). The reaction mixture was stirred at 23° C. for 0.5 h. Then DCM and excess SOCl₂ were removed under reduced pressure. The obtained intermediate product was dissolved in dry DMF (10 mL) and potassium phthalimide (0.15 g, 0.79 mmol, 1.2 equiv) was added. The mixture was stirred at 80° C. for 1 h. The reaction mixture was cooled to 23° C. and DMF was removed under reduced pressure. Then water (30 mL) was added, and the mixture was extracted by DCM (30 mL) for three times. The organic phase was combined, dried over anhydrous MgSO₄ and filtered. The filtrate was concentrated and purified by column chromatography (SiO₂) with hexane/EtOAc=20/1 as the mobile phase to give the title compound as a yellow solid (0.30 g, 75%).

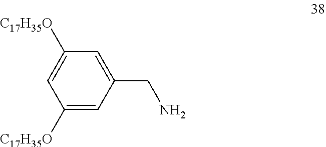

Compound 38. Compound 37 (0.21 g, 0.32 mmol, 1 equiv) and hydrazine monohydrate (NH₂NH₂·H₂O, 0.16 g, 3.17 mmol, 10 equiv) were dissolved in EtOH (10 mL). The reaction mixture was heated at reflux for 2 h. The mixture was cooled to 23° C. and the solvent was removed under reduced pressure. Then water (10 mL) was added, and the mixture was extracted by DCM (10 mL) for three times. The organic phases were combined, dried over anhydrous MgSO₄ and filtered. Filtration and evaporation of the solvent yielded the title compound as a colorless oil (0.20 g, 99%).

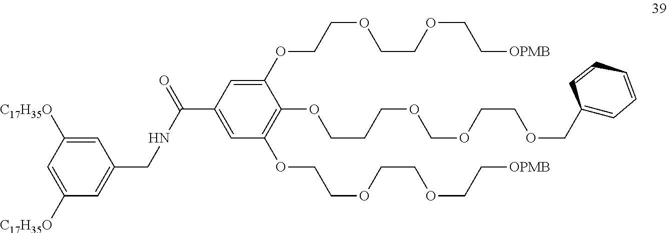

Compound 39. Compound 39 was synthesized from compound 38 (70 mg, 0.13 mmol) and compound 33 (0.12 g, 0.13 mmol) following a procedure similar to that used for the synthesis of compound 34a. The title compound was obtained as a colorless oil (0.14 g, 78%).

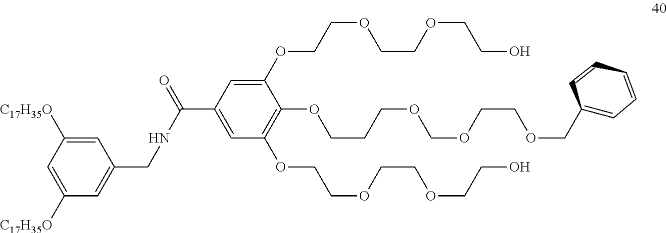

Compound 40. Compound 40 was synthesized from compound 39 (0.14 g, 0.098 mmol) and DDQ (56 mg, 0.25 mmol) following a procedure similar to that used for the synthesis of compound 35a. The title compound was obtained as a colorless oil (80 mg, 69%).

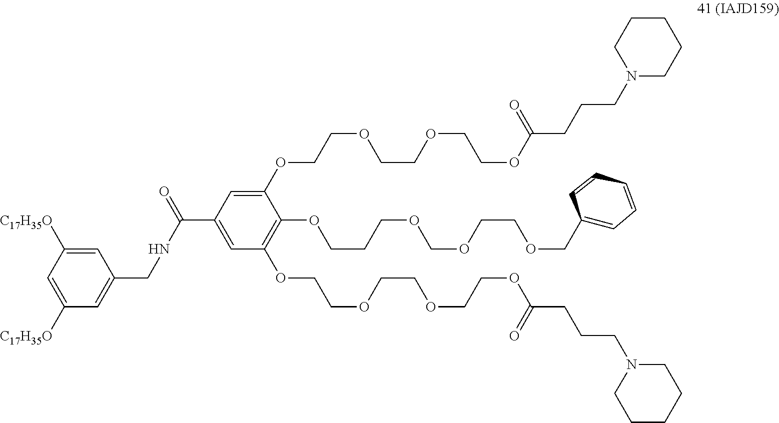

Compound 41 (IAJD159). Compound 41 was synthesized from compound 40 (80 mg, 0.068 mmol), 4-(piperidin-1-yl)butanoic acid hydrochloride (36 mg, 0.17 mmol) and DCC (70 mg, 0.34 mmol) following a procedure similar to that used for the synthesis of compound 36a. The title compound was obtained as a colorless oil (77 mg, 77%).

The dimensions of DNPs co-assembled from IAJDs and Luc-mRNA by injection in acetate buffer (10 mM, pH 4.0) and luminescence expression results in HEK 293T cells (125 ng/well, n=3) is shown in the Table 16 below.

TABLE 16

| | Buffer | C_{IAJD} | C_{Luc-mRNA} | Size | | | Standard |
|---|---|---|---|---|---|---|---|
| IAJD | (10 mM) | (mg/mL) | (mg/mL) | (d, nm) | PDI | Luminescence | deviation |
| 66^{a} | Acetate | 4 | 0.10 | 165 | 0.320 | 234 | 43 |
| | (pH 4.0) | | | | | | |
| 71^{a} | Acetate | 4 | 0.10 | 237 | 0.451 | 473,548 | 123,589 |
| | (pH 4.0) | | | | | | |
| 81^{a} | Acetate | 4 | 0.10 | 111 | 0.287 | 206,161 | 21,200 |
| | (pH 4.0) | | | | | | |
| 86^{a} | Acetate | 4 | 0.10 | 91 | 0.234 | 47,066 | 21,163 |
| | (pH 4.0) | | | | | | |
| 105^{a} | Acetate | 4 | 0.10 | 94 | 0.294 | 85 | 21 |
| | (pH 4.0) | | | | | | |
| 106^{a} | Acetate | 4 | 0.10 | 105 | 0.275 | 309 | 116 |
| | (pH 4.0) | | | | | | |
| 107^{a} | Acetate | 4 | 0.10 | 146 | 0.279 | 93,745 | 12,116 |
| | (pH 4.0) | | | | | | |
| 113 | Acetate | 4 | 0.10 | 181 | 0.511 | 78,118 | 13,787 |
| | (pH 4.0) | | | | | | |
| 114 | Acetate | 4 | 0.10 | 105 | 0.351 | 1,580 | 1,541 |
| | (pH 4.0) | | | | | | |
| 115 | Acetate | 4 | 0.10 | 123 | 0.367 | 390,021 | 17,022 |
| | (pH 4.0) | | | | | | |
| 116 | Acetate | 4 | 0.10 | 150 | 0.264 | 192,213 | 35,528 |
| | (pH 4.0) | | | | | | |
| 117 | Acetate | 4 | 0.10 | 255 | 0.367 | 516,522 | 54,783 |
| | (pH 4.0) | | | | | | |
| 118 | Acetate | 4 | 0.10 | 104 | 0.278 | 568,354 | 15,820 |
| | (pH 4.0) | | | | | | |
| 119 | Acetate | 4 | 0.10 | 163 | 0.459 | 565,906 | 39,932 |
| | (pH 4.0) | | | | | | |
| 120 | Acetate | 4 | 0.10 | 212 | 0.474 | 443,836 | 64,704 |
| | (pH 4.0) | | | | | | |
| 122 | Acetate | 4 | 0.10 | 215 | 0.337 | 55,101 | 3,169 |
| | (pH 4.0) | | | | | | |
| 124 | Acetate | 4 | 0.10 | 276 | 0.280 | 145,115 | 5,020 |
| | (pH 4.0) | | | | | | |
| 125 | Acetate | 4 | 0.10 | 255 | 0.300 | 119,405 | 21,570 |
| | (pH 4.0) | | | | | | |
| 126 | Acetate | 4 | 0.10 | 158 | 0.247 | 675 | 137 |
| | (pH 4.0) | | | | | | |
| 127 | Acetate | 4 | 0.10 | 276 | 0.270 | 147,731 | 24,320 |
| | (pH 4.0) | | | | | | |
| 128 | Acetate | 4 | 0.10 | 291 | 0.381 | 449,311 | 104,462 |
| | (pH 4.0) | | | | | | |
| 130 | Acetate | 4 | 0.10 | 172 | 0.271 | 76,345 | 5,712 |
| | (pH 4.0) | | | | | | |
| 133 | Acetate | 4 | 0.10 | 228 | 0.414 | 107,307 | 17,263 |
| | (pH 4.0) | | | | | | |
| 135 | Acetate | 4 | 0.10 | 92 | 0.267 | 432 | 236 |
| | (pH 4.0) | | | | | | |
| 136 | Acetate | 4 | 0.10 | 149 | 0.307 | 283,656 | 146,748 |
| | (pH 4.0) | | | | | | |
| 138 | Acetate | 4 | 0.10 | 99 | 0.305 | 102,620 | 5,680 |
| | (pH 4.0) | | | | | | |
| 141 | Acetate | 4 | 0.10 | 158 | 0.313 | 140,589 | 107,411 |
| | (pH 4.0) | | | | | | |
| 142 | Acetate | 4 | 0.10 | 230 | 0.293 | 463,750 | 213,429 |
| | (pH 4.0) | | | | | | |
| 143 | Acetate | 4 | 0.10 | 169 | 0.270 | 71,851 | 10,736 |
| | (pH 4.0) | | | | | | |
| 144 | Acetate | 4 | 0.10 | 236 | 0.432 | 253,672 | 25,719 |
| | (pH 4.0) | | | | | | |
| 145 | Acetate | 4 | 0.10 | 194 | 0.306 | 296 | 353 |
| | (pH 4.0) | | | | | | |
| 146 | Acetate | 4 | 0.10 | 231 | 0.276 | 50,625 | 6,496 |
| | (pH 4.0) | | | | | | |
| 147 | Acetate | 4 | 0.10 | 144 | 0.287 | 5,844 | 1,645 |
| | (pH 4.0) | | | | | | |
| 148 | Acetate | 4 | 0.10 | 363 | 0.339 | 139,420 | 10,045 |
| | (pH 4.0) | | | | | | |
| 149 | Acetate | 4 | 0.10 | 193 | 0.304 | 149,935 | 22,183 |
| | (pH 4.0) | | | | | | |
| 150 | Acetate | 4 | 0.10 | 285 | 0.454 | 134,225 | 13,323 |
| | (pH 4.0) | | | | | | |
| 151 | Acetate | 4 | 0.10 | 287 | 0.216 | 9,310 | 1,977 |
| | (pH 4.0) | | | | | | |
| 152 | Acetate | 4 | 0.10 | 281 | 0.370 | 88,202 | 24,833 |
| | (pH 4.0) | | | | | | |
| 153 | Acetate | 4 | 0.10 | 232 | 0.238 | 8,860 | 2,558 |
| | (pH 4.0) | | | | | | |
| 154 | Acetate | 4 | 0.10 | 429 | 0.407 | 17,871 | 6,419 |
| | (pH 4.0) | | | | | | |
| 161 | Acetate | 4 | 0.10 | 129 | 0.235 | 128 | 49 |
| | (pH 4.0) | | | | | | |
| 162 | Acetate | 4 | 0.10 | 191 | 0.301 | 222,235 | 32,067 |
| | (pH 4.0) | | | | | | |
| 171 | Acetate | 4 | 0.10 | 174 | 0.268 | 1,436 | 832 |
| | (pH 4.0) | | | | | | |
| 172 | Acetate | 4 | 0.10 | 235 | 0.203 | 166,377 | 22,769 |
| | (pH 4.0) | | | | | | |
| 173 | Acetate | 4 | 0.10 | 139 | 0.301 | 2,302 | 410 |
| | (pH 4.0) | | | | | | |
| 177 | Acetate | 4 | 0.10 | 159 | 0.275 | 4,343 | 755 |
| | (pH 4.0) | | | | | | |
| 178 | Acetate | 4 | 0.10 | 344 | 0.223 | 235,419 | 43,906 |
| | (pH 4.0) | | | | | | |
| 110 | Acetate | 4 | 0.10 | 111 | 0.274 | 47 | 30 |
| | (pH 4.0) | | | | | | |
| 111 | Acetate | 4 | 0.10 | 99 | 0.232 | 41 | 35 |
| | (pH 4.0) | | | | | | |
| 155 | Acetate | 4 | 0.10 | 83 | 0.279 | 74 | 49 |
| | (pH 4.0) | | | | | | |
| 156 | Acetate | 4 | 0.10 | 75 | 0.236 | 59 | 23 |
| | (pH 4.0) | | | | | | |
| 157 | Acetate | 4 | 0.10 | 68 | 0.238 | 47 | 7 |
| | (pH 4.0) | | | | | | |
| 158 | Acetate | 4 | 0.10 | 75 | 0.246 | 83 | 36 |
| | (pH 4.0) | | | | | | |
| 159 | Acetate | 4 | 0.10 | 91 | 0.218 | 24,055 | 15,173 |
| | (pH 4.0) | | | | | | |
| Negative | | | | | | 81 | 5 |
| Control | | | | | | | |
| (untreated | | | | | | | |
| cells) | | | | | | | |
| MC3-Luc- | | | | | | 1,320,533 | 105,967 |
| mRNA | | | | | | | |

^{a}The synthesis and characterizations of IAJDs 66, 71, 81, 86, 105, 106, and 107 as well as the dimensions of DNPs and the luminescence expression results in HEK 293T cells are available in literature recently reported (Zhang et al., J. Am. Chem. Soc. 2021, 143, 17975-17982).

### 5. Luminescence Expression In Vivo

The Table 17 below shows the quantification of luminescence expression in vivo.

TABLE 17

| | | | | | | | | Total | |
|---|---|---|---|---|---|---|---|---|---|
| | C_{IAJD} | C_{Luc-mRNA} | pH | | Size | | T | flux | Organs with |
| IAJD | (mg/mL) | (mg/mL) | Acetate | Sample | (d, nm) | PDI | (h)^{a} | (p/s) | luminescence^{b} |
| 66 | 4 | 0.10 | 4.0 | 4.50 | 165 | 0.320 | 4 | 2.58 × | Lung (weak), |
| | | | | | | | | 10⁷ | liver (strong), |
| | | | | | | | | | spleen (strong) |
| 71 | 4 | 0.10 | 4.0 | 4.39 | 237 | 0.451 | 5 | 3.79 × | Lung (weak), |
| | | | | | | | | 10⁷ | liver (weak), |
| | | | | | | | | | spleen (strong) |
| 81 | 4 | 0.10 | 4.0 | 4.38 | 111 | 0.287 | 6 | 2.32 × | Lung (weak), |
| | | | | | | | | 10⁷ | liver (strong), |
| | | | | | | | | | spleen (weak) |
| 86 | 4 | 0.10 | 4.0 | 4.39 | 91 | 0.234 | 5 | 4.73 × | Lung (weak), |
| | | | | | | | | 10⁷ | liver (strong), |
| | | | | | | | | | spleen (weak) |
| 105 | 4 | 0.10 | 4.0 | 4.42 | 94 | 0.294 | 6 | 4.74 × | Lung (weak), |
| | | | | | | | | 10⁷ | liver (strong), |
| | | | | | | | | | spleen (weak) |
| 106 | 4 | 0.10 | 4.0 | 4.41 | 105 | 0.275 | 6 | 3.48 × | Lung (weak), |
| | | | | | | | | 10⁷ | liver (weak), |
| | | | | | | | | | spleen (strong) |
| 107 | 4 | 0.10 | 4.0 | 4.42 | 146 | 0.279 | 6 | 2.19 × | Lung (weak), |
| | | | | | | | | 10⁷ | liver (weak), |
| | | | | | | | | | spleen (strong) |
| 113 | 4 | 0.10 | 4.0 | 4.48 | 181 | 0.511 | 5 | 1.70 × | Spleen (strong) |
| | | | | | | | | 10⁷ | |
| 114 | 4 | 0.10 | 4.0 | 4.46 | 105 | 0.351 | 5 | 3.56 × | Lung (weak), |
| | | | | | | | | 10⁶ | liver (weak), |
| | | | | | | | | | spleen (strong) |
| 115 | 4 | 0.10 | 4.0 | 4.44 | 123 | 0.367 | 5 | 4.59 × | Lung (weak), |
| | | | | | | | | 10⁶ | spleen (strong) |
| 116 | 4 | 0.10 | 4.0 | 4.47 | 150 | 0.264 | 5 | 2.38 × | Lung (weak), |
| | | | | | | | | 10⁷ | liver (weak), |
| | | | | | | | | | spleen (strong) |
| 117 | 4 | 0.10 | 4.0 | 4.40 | 255 | 0.367 | 5 | 8.64 × | Lung (weak), |
| | | | | | | | | 10⁶ | liver (weak), |
| | | | | | | | | | spleen (strong) |
| 118 | 4 | 0.10 | 4.0 | 4.43 | 104 | 0.278 | 6 | 1.46 × | Lung (weak), |
| | | | | | | | | 10⁷ | liver (weak), |
| | | | | | | | | | spleen (strong) |
| 119 | 4 | 0.10 | 4.0 | 4.36 | 163 | 0.459 | 5 | 8.22 × | Lung (weak), |
| | | | | | | | | 10⁷ | liver (weak), |
| | | | | | | | | | spleen (strong) |
| 120 | 4 | 0.10 | 4.0 | 4.40 | 212 | 0.474 | 5 | 2.48 × | Spleen (strong) |
| | | | | | | | | 10⁷ | |
| 122 | 4 | 0.10 | 4.0 | 4.49 | 215 | 0.337 | 5 | 2.50 × | Lung (weak), |
| | | | | | | | | 10⁷ | spleen (strong) |
| 124 | 4 | 0.10 | 4.0 | 4.51 | 276 | 0.280 | 5 | 3.77 × | Lung (weak), |
| | | | | | | | | 10⁷ | spleen (strong) |
| 125 | 4 | 0.10 | 4.0 | 4.44 | 255 | 0.300 | 5 | 2.15 × | Lung (weak), |
| | | | | | | | | 10⁸ | liver (weak), |
| | | | | | | | | | spleen (strong) |
| 126 | 4 | 0.10 | 4.0 | 4.48 | 158 | 0.247 | 5 | 3.21 × | Lung (weak), |
| | | | | | | | | 10⁷ | liver (weak), |
| | | | | | | | | | spleen (strong) |
| 127 | 4 | 0.10 | 4.0 | 4.46 | 276 | 0.270 | 5 | 1.07 × | Lung (weak), |
| | | | | | | | | 10⁸ | liver (weak), |
| | | | | | | | | | spleen (strong) |
| 128 | 4 | 0.10 | 4.0 | 4.40 | 291 | 0.381 | 5 | 5.53 × | Lung (weak), |
| | | | | | | | | 10⁷ | liver (weak), |
| | | | | | | | | | spleen (strong) |
| 130 | 4 | 0.10 | 4.0 | 4.50 | 172 | 0.271 | 5 | 1.18 × | Lung (weak), |
| | | | | | | | | 10⁸ | liver (weak), |
| | | | | | | | | | spleen (strong) |
| 133 | 4 | 0.10 | 4.0 | 4.63 | 228 | 0.414 | 6 | 1.11 × | Lung (strong), |
| | | | | | | | | 10⁷ | spleen (weak) |
| 135 | 4 | 0.10 | 4.0 | 4.95 | 92 | 0.267 | 5 | 1.22 × | Lung (weak), |
| | | | | | | | | 10⁷ | liver (strong), |
| | | | | | | | | | spleen (weak) |
| 136 | 4 | 0.10 | 4.0 | 4.57 | 149 | 0.307 | 5 | 1.04 × | Lung (weak), |
| | | | | | | | | 10⁷ | liver (weak), |
| | | | | | | | | | spleen (strong) |
| 138 | 4 | 0.10 | 4.0 | 4.52 | 99 | 0.305 | 5 | 1.49 × | Lung (weak), |
| | | | | | | | | 10⁷ | spleen (strong) |
| 141 | 4 | 0.10 | 4.0 | 4.62 | 158 | 0.313 | 5 | 2.06 × | Lung (weak), |
| | | | | | | | | 10⁷ | liver (strong), |
| | | | | | | | | | spleen (strong) |
| 142 | 4 | 0.10 | 4.0 | 4.52 | 230 | 0.293 | 5 | 2.51 × | Lung (weak), |
| | | | | | | | | 10⁷ | liver (strong), |
| | | | | | | | | | spleen (strong) |
| 143 | 4 | 0.10 | 4.0 | 4.48 | 169 | 0.270 | 5 | 1.28 × | Lung (strong), |
| | | | | | | | | 10⁷ | liver (strong), |
| | | | | | | | | | spleen (strong) |
| 144 | 4 | 0.10 | 4.0 | 4.40 | 236 | 0.432 | 5 | 1.39 × | Lung (weak), |
| | | | | | | | | 10⁷ | liver (weak), |
| | | | | | | | | | spleen (strong) |
| 145 | 4 | 0.10 | 4.0 | 4.51 | 194 | 0.306 | 5 | 9.25 × | Lung (strong), |
| | | | | | | | | 10⁶ | liver (weak), |
| | | | | | | | | | spleen (strong) |
| 146 | 4 | 0.10 | 4.0 | 4.40 | 231 | 0.276 | 5 | 4.70 × | Lung (strong), |
| | | | | | | | | 10⁷ | liver (strong), |
| | | | | | | | | | spleen (strong) |
| 147 | 4 | 0.10 | 4.0 | 4.50 | 144 | 0.287 | 5 | 9.24 × | Lung (weak), |
| | | | | | | | | 10⁶ | liver (weak), |
| | | | | | | | | | spleen (strong) |
| 148 | 4 | 0.10 | 4.0 | 4.46 | 363 | 0.339 | 5 | 6.33 × | Lung (weak), |
| | | | | | | | | 10⁷ | liver (weak), |
| | | | | | | | | | spleen (strong) |
| 149 | 4 | 0.10 | 4.0 | 4.50 | 193 | 0.304 | 5 | 2.26 × | Lung (weak), |
| | | | | | | | | 10⁷ | liver (weak), |
| | | | | | | | | | spleen (strong) |
| 150 | 4 | 0.10 | 4.0 | 4.51 | 285 | 0.454 | 5 | 3.47 × | Lung (weak), |
| | | | | | | | | 10⁷ | liver (weak), |
| | | | | | | | | | spleen (strong) |
| 151 | 4 | 0.10 | 4.0 | 4.57 | 287 | 0.216 | 5 | 3.12 × | Lung (weak), |
| | | | | | | | | 10⁷ | liver (weak), |
| | | | | | | | | | spleen (strong) |
| 152 | 4 | 0.10 | 4.0 | 4.45 | 281 | 0.370 | 5 | 1.97 × | Liver (weak), |
| | | | | | | | | 10⁷ | spleen (strong) |
| 153 | 4 | 0.10 | 4.0 | 4.52 | 232 | 0.238 | 6 | 7.33 × | Lung (weak), |
| | | | | | | | | 10⁷ | liver (strong), |
| | | | | | | | | | spleen (strong) |
| 154 | 4 | 0.10 | 4.0 | 4.47 | 429 | 0.407 | 6 | 2.39 × | Lung (weak), |
| | | | | | | | | 10⁷ | liver (weak), |
| | | | | | | | | | spleen (strong) |
| 161 | 4 | 0.10 | 4.0 | 4.97 | 129 | 0.235 | 5 | 8.48 × | Lung (weak), |
| | | | | | | | | 10⁷ | liver (strong), |
| | | | | | | | | | spleen (strong) |
| 162 | 4 | 0.10 | 4.0 | 4.66 | 191 | 0.301 | 5 | 2.03 × | Lung (weak), |
| | | | | | | | | 10⁸ | liver (weak), |
| | | | | | | | | | spleen (strong) |
| 171 | 4 | 0.10 | 4.0 | 4.88 | 174 | 0.268 | 5 | 7.68 × | Lung (weak), |
| | | | | | | | | 10⁷ | liver (strong), |
| | | | | | | | | | spleen (strong) |
| 172 | 4 | 0.10 | 4.0 | 4.84 | 235 | 0.203 | 5 | 6.82 × | Lung (weak), |
| | | | | | | | | 10⁷ | liver (weak), |
| | | | | | | | | | spleen (strong) |
| 173 | 4 | 0.10 | 4.0 | 4.79 | 139 | 0.301 | 5 | 1.23 × | Lung (weak), |
| | | | | | | | | 10⁸ | liver (strong), |
| | | | | | | | | | spleen (weak) |
| 177 | 4 | 0.10 | 4.0 | 4.76 | 159 | 0.275 | 6 | 9.18 × | Lung (weak), |
| | | | | | | | | 10⁷ | liver (strong), |
| | | | | | | | | | spleen (strong) |
| 178 | 4 | 0.10 | 4.0 | 4.58 | 344 | 0.223 | 6 | 4.05 × | Lung (weak), |
| | | | | | | | | 10⁸ | liver (weak), |
| | | | | | | | | | spleen (strong) |
| 110 | 4 | 0.10 | 4.0 | 4.91 | 111 | 0.274 | 5 | 8.85 × | Lung (strong) |
| | | | | | | | | 10⁷ | |
| 111 | 4 | 0.10 | 4.0 | 4.84 | 99 | 0.232 | 5 | 7.21 × | Lung (strong), |
| | | | | | | | | 10⁷ | spleen (weak) |
| 155 | 4 | 0.10 | 4.0 | 4.93 | 83 | 0.279 | 5 | 1.01 × | Lung (strong), |
| | | | | | | | | 10⁸ | spleen (weak) |
| 156 | 4 | 0.10 | 4.0 | 4.82 | 75 | 0.236 | 5 | 5.83 × | Lung (strong) |
| | | | | | | | | 10⁶ | |
| 157 | 4 | 0.10 | 4.0 | 4.84 | 68 | 0.238 | 5 | 3.66 × | Lung (strong) |
| | | | | | | | | 10⁷ | |
| 158 | 4 | 0.10 | 4.0 | 4.83 | 75 | 0.246 | 5 | 1.23 × | Lung (strong) |
| | | | | | | | | 10⁷ | |
| 159 | 4 | 0.10 | 4.0 | 4.72 | 91 | 0.218 | 6 | 6.86 × | Lung (strong) |
| | | | | | | | | 10⁷ | |

^{a}Time from injection of DNP (co-assembled from IAJDs and Luc-mRNA) solution to the imaging of mice on the IVIS imaging system.
^{b}Organs showing luminescence indicated the expression of luciferase enzyme in the corresponding organs which interacted with D-Luciferin to generate oxy-Luciferin emitting light. Strong meant the luminescence intensity in that organ was relatively high, compared to other organs. The organ highlighted with green color meant it showed strongest luminescence intensity. Weak meant the luminescence intensity in that organ was relatively low, compared to other organs.

### 6. Comparison of Luminescence Expression In Vivo by Symmetric IAJDs and Nonsymmetric IAJDs

The Table 18 below shows in vivo activity of symmetric IAJDs with identical number of carbons on each alkyl chain

TABLE 18

| | Number of | Average number | | |
|---|---|---|---|---|
| | carbons on each | of carbons per | Ionizable | In vivo activity |
| IAJD^{a} | alkyl chain | alkyl chain | amine^{b} | (total flux, p/s) |
| 76 | 8 + 8 (EH + EH) | 8 | MP | 7.81 × 10⁷ |
| 87 | 8 + 8 (EH + EH) | 8 | HP | 3.24 × 10⁷ |
| 88 | 8 + 8 | 8 | MP | 2.34 × 10⁶ |
| 89 | 8 + 8 | 8 | HP | 5.92 × 10⁷ |
| 77 | 9 + 9 | 9 | MP | 2.44 × 10⁶ |
| 91 | 10 + 10 | 10 | HP | 9.94 × 10⁶ |
| 66 | 12 + 12 | 12 | MP | 2.58 × 10⁷ |
| 71 | 12 + 12 | 12 | HP | 3.79 × 10⁷ |
| 74 | 14 + 14 | 14 | MP | 1.71 × 10⁷ |
| 75 | 16 + 16 | 16 | MP | 3.37 × 10⁷ |
| 98 | 18 + 18 | 18 | MP | 2.06 × 10⁶ |
| 99 | 18 + 18 | 18 | HP | 4.49 × 10⁶ |

^{a}The synthesis and characterizations of IAJDs 66, 71, 74, 75, 76, 77, 87, 88, 89, 91, 98 and 99 as well as their luminescence expression results in vivo were available in the literature as recently reported.
^{b}MP: methyl piperazine; HP: hydroxy piperazine.

The Table 19 below shows in vivo activity of nonsymmetric IAJDs with different number of carbons on each alkyl chain.

TABLE 19

| | Number of | Average number | | |
|---|---|---|---|---|
| | carbons on | of carbons per | Ionizable | In vivo activity |
| IAJD | each alkyl chain | alkyl chain | aminea | (total flux, p/s) |
| 81 | 11 + 18 | 14.5 | MP | 2.32 × 10⁷ |
| 86 | 11 + 16 | 13.5 | MP | 4.73 × 10⁷ |
| 105 | 11 + 14 | 12.5 | MP | 4.74 × 10⁷ |
| 106 | 12 + 16 | 14 | MP | 3.48 × 10⁷ |
| 107 | 12 + 18 | 15 | MP | 2.19 × 10⁷ |
| 113 | 11 + 8 | 9.5 | HP | 1.70 × 10⁷ |
| 114 | 11 + 10 | 10.5 | MP | 3.56 × 10⁶ |
| 115 | 11 + 10 | 10.5 | HP | 4.59 × 10⁶ |
| 116 | 11 + 13 | 12 | MP | 2.38 × 10⁷ |
| 117 | 11 + 13 | 12 | HP | 8.64 × 10⁶ |
| 118 | 11 + 15 | 13 | MP | 1.46 × 10⁷ |
| 119 | 11 + 15 | 13 | HP | 8.22 × 10⁷ |
| 120 | 11 + 16 | 13.5 | HP | 2.48 × 10⁷ |
| 122 | 11 + 9 | 10 | HP | 2.50 × 10⁷ |
| 124 | 11 + 12 | 11.5 | HP | 3.77 × 10⁷ |
| 125 | 11 + 14 | 12.5 | HP | 2.15 × 10⁸ |
| 126 | 11 + 17 | 14 | MP | 3.21 × 10⁷ |
| 127 | 11 + 17 | 14 | HP | 1.07 × 10⁸ |
| 128 | 11 + 18 | 14.5 | HP | 5.53 × 10⁷ |
| 130 | 11 + 8 | 9.5 | HP | 1.18 × 10⁸ |
| | (11 + EH) | | | |
| 135 | 15 + 8 | 11.5 | MP | 1.22 × 10⁷ |
| | (15 + EH) | | | |
| 136 | 15 + 8 | 11.5 | HP | 1.04 × 10⁷ |
| | (15 + EH) | | | |
| 138 | 15 + 8 | 11.5 | HP | 1.49 × 10⁷ |
| 141 | 15 + 10 | 12.5 | MP | 2.06 × 10⁷ |
| 142 | 15 + 10 | 12.5 | HP | 2.51 × 10⁷ |
| 143 | 15 + 12 | 13.5 | MP | 1.28 × 10⁷ |
| 144 | 15 + 12 | 13.5 | HP | 1.39 × 10⁷ |
| 145 | 15 + 13 | 14 | MP | 9.25 × 10⁶ |
| 146 | 15 + 13 | 14 | HP | 4.70 × 10⁷ |
| 147 | 15 + 14 | 14.5 | MP | 9.24 × 10⁶ |
| 148 | 15 + 14 | 14.5 | HP | 6.33 × 10⁷ |
| 149 | 15 + 16 | 15.5 | MP | 2.26 × 10⁷ |
| 150 | 15 + 16 | 15.5 | HP | 3.47 × 10⁷ |
| 151 | 15 + 17 | 16 | MP | 3.12 × 10⁷ |
| 152 | 15 + 17 | 16 | HP | 1.97 × 10⁷ |
| 153 | 15 + 18 | 16.5 | MP | 7.33 × 10⁷ |
| 154 | 15 + 18 | 16.5 | HP | 2.39 × 10⁷ |
| 161 | 13 + 8 | 10.5 | MP | 8.48 × 10⁷ |
| | (13 + EH) | | | |
| 162 | 13 + 8 | 10.5 | HP | 2.03 × 10⁸ |
| | (13 + EH) | | | |
| 171 | 13 + 14 | 13.5 | MP | 7.68 × 10⁷ |
| 172 | 13 + 14 | 13.5 | HP | 6.82 × 10⁷ |
| 173 | 13 + 16 | 14.5 | MP | 1.23 × 10⁸ |
| 177 | 13 + 18 | 15.5 | MP | 9.18 × 10⁷ |
| 178 | 13 + 18 | 15.5 | HP | 4.05 × 10⁸ |

The Table 20 below shows representative ratios between in vivo activities of nonsymmetric IAJDs and corresponding symmetric IJDs.

TABLE 20

| | | | | Ratio between |
|---|---|---|---|---|
| | In vivo | | In vivo | in vivo |
| | activity | | activity | activities |
| | (total | Corresponding | (total | (nonsymmetric/ |
| Nonsymmetric | flux, | symmetric | flux, | symmetric |
| IAJDs | p/s) | IAJDs | p/s) | IAJDs) |
| 125 (11/14-HP) | 2.15 × 10⁸ | 71 (12/12-HP) | 3.79 × 10⁷ | IAJD125/ |
| | | | | IAJD71 = 5.7 |
| 130 (11/EH-HP) | 1.18 × 10⁸ | 87 (EH/EH-HP) | 3.24 × 10⁷ | IAJD130/ |
| | | | | IAJD87 = 3.6 |
| 162 (13/EH-HP) | 2.03 × 10⁸ | 87 (EH/EH-HP) | 3.24 × 10⁷ | IAJD162/ |
| | | | | IAJD87 = 6.3 |
| 171 (13/14-MP) | 7.68 × 10⁷ | 74 (14/14-MP) | 1.71 × 10⁷ | IAJD171/ |
| | | | | IAJD74 = 4.5 |
| 173 (13/16-MP) | 1.23 × 10⁸ | 75 (16/16-MP) | 3.37 × 10⁷ | IAJD173/ |
| | | | | IAJD75 = 3.6 |
| 81 (11/18-MP) | 2.32 × 10⁷ | 98 (18/18-MP) | 2.06 × 10⁶ | IAJD81/ |
| | | | | IAJD98 = 11.3 |
| 128 (11/18-HP) | 5.53 × 10⁷ | 99 (18/18-HP) | 4.49 × 10⁶ | IAJD128/ |
| | | | | IAJD99 = 12.3 |
| 177 (13/18-MP) | 9.18 × 10⁷ | 98 (18/18-MP) | 2.06 × 10⁶ | IAJD177/ |
| | | | | IAJD98 = 44.6 |
| 178 (13/18-HP) | 4.05 × 10⁸ | 99 (18/18-HP) | 4.49 × 10⁶ | IAJD178/ |
| | | | | IAJD99 = 90.2 |
| 153 (15/18-MP) | 7.33 × 10⁷ | 98 (18/18-MP) | 2.06 × 10⁶ | IAJD153/ |
| | | | | IAJD98 = 35.6 |
| 154 (15/18-HP) | 2.39 × 10⁷ | 99 (18/18-HP) | 4.49 × 10⁶ | IAJD154/ |
| | | | | IAJD99 = 5.3 |
| MC3-Luc-mRNA | 17.0 × 10⁸ | 178 (13/18-HP) | 4.05 × 10⁸ | MC3/178 = 4.2 |

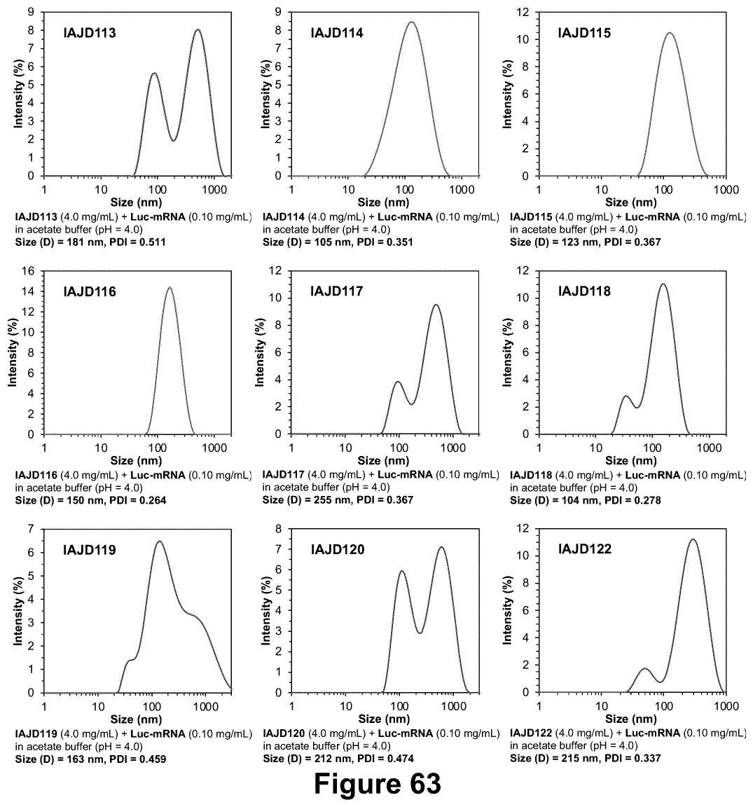

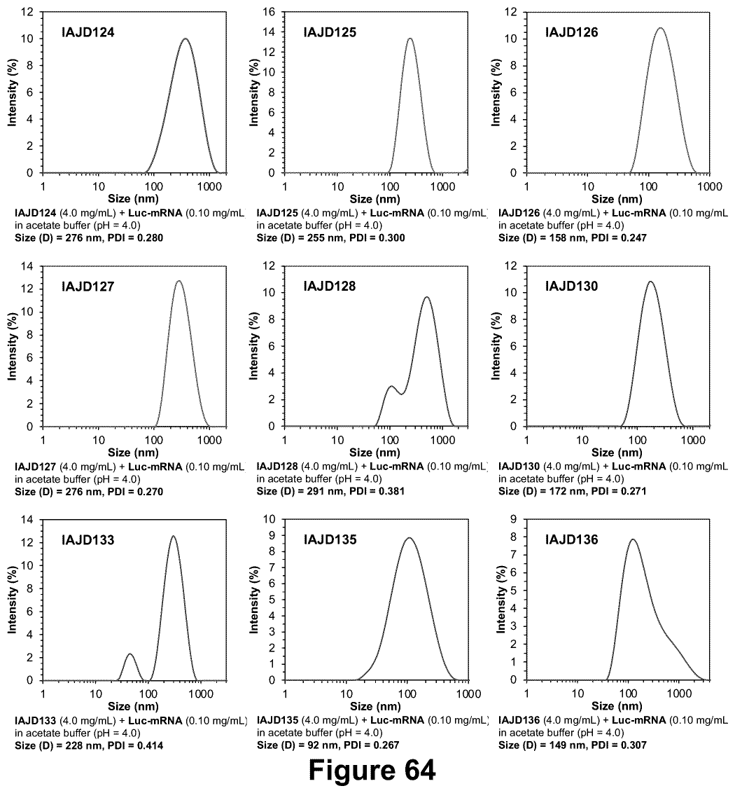

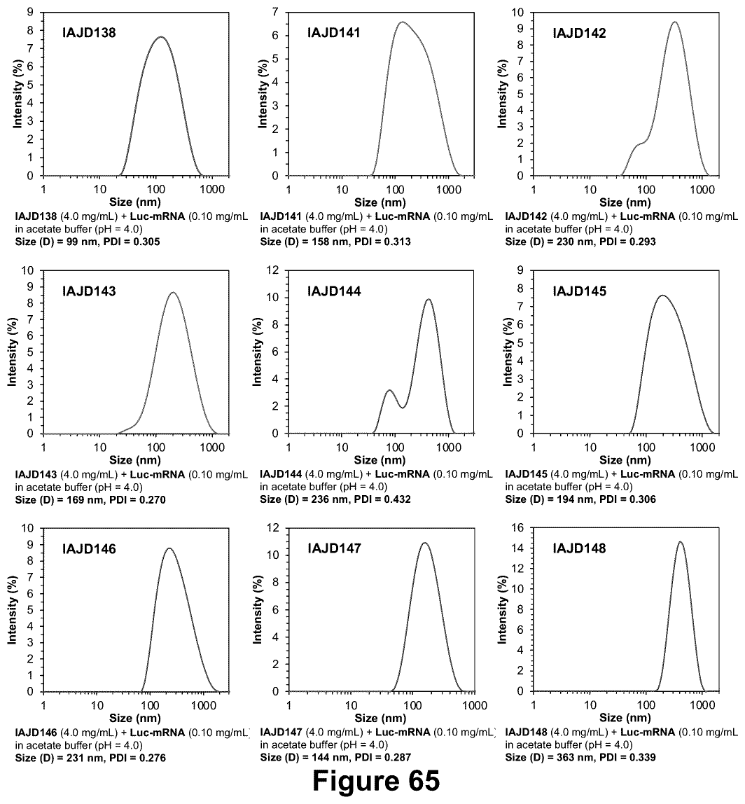

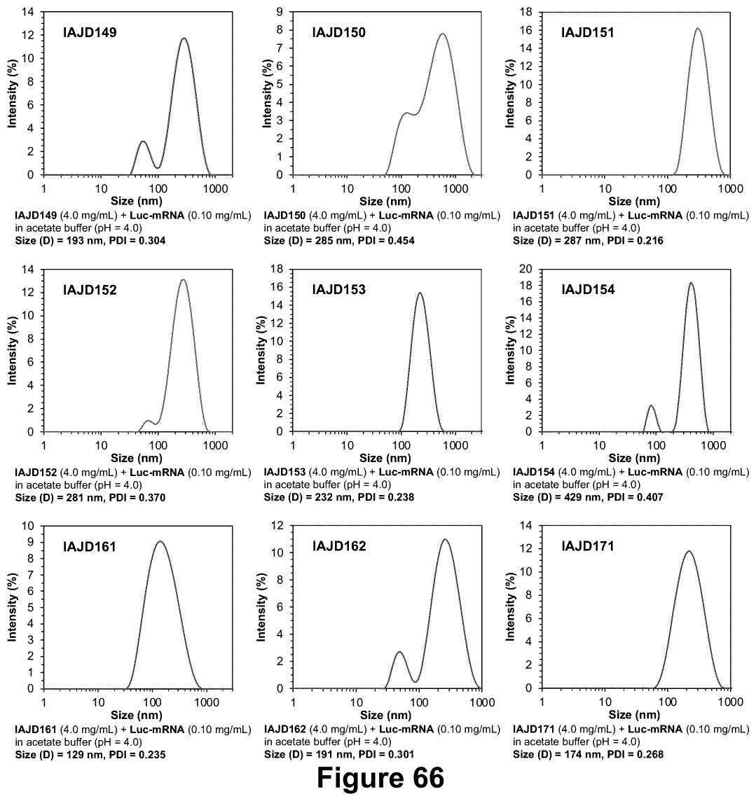

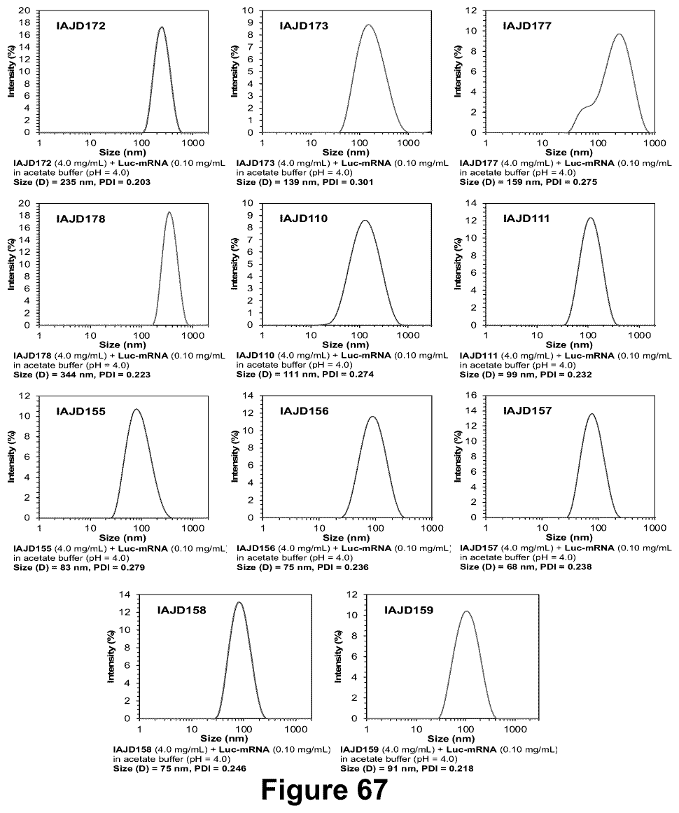

The DLS data of DNPs co-assembled from IAJDs and Luc-mRNA is shown in FIG. 63 through FIG. 67. The stability of DNPs by DLS is shown in the Table 21 below.

TABLE 21
Stability of DNPs Determined by DLS After Different Periods of Time at 5° C.

| IAJD | | | pH | | Original | | | | |
|---|---|---|---|---|---|---|---|---|---|
| (storage | C_{IAJD} | C_{Luc-mRNA} | Acetate | | size | | Size | | Ranking of |
| time) | (mg/mL) | (mg/mL) | buffer | Sample | (d, nm) | PDI | (d, nm) | PDI | stability^{c} |
| 113 (70 | 4.0 | 0.1 | 4.00 | 4.48 | 181 | 0.511 | 184 | 0.365 | Excellent |
| days) | | | | | (B)^{b} | | (B) | | |
| 114 (75 | 4.0 | 0.1 | 4.00 | 4.46 | 105 | 0.351 | 107 | 0.369 | Good |
| days) | | | | | (U) | | (B) | | |
| 115 (70 | 4.0 | 0.1 | 4.00 | 4.44 | 123 | 0.367 | 134 | 0.383 | Excellent |
| days) | | | | | (U) | | (U) | | |
| 116 (62 | 4.0 | 0.1 | 4.00 | 4.47 | 150 | 0.264 | 168 | 0.272 | Excellent |
| days) | | | | | (U) | | (U) | | |
| 117 (70 | 4.0 | 0.1 | 4.00 | 4.40 | 255 | 0.367 | 323 | 0.312 | Excellent |
| days) | | | | | (B) | | (B) | | |
| 118 (75 | 4.0 | 0.1 | 4.00 | 4.43 | 104 | 0.278 | 105 | 0.262 | Excellent |
| days) | | | | | (B) | | (B) | | |
| 119 (62 | 4.0 | 0.1 | 4.00 | 4.36 | 163 | 0.459 | 254 | 0.489 | Good |
| days) | | | | | (M) | | (B) | | |
| 120 (70 | 4.0 | 0.1 | 4.00 | 4.40 | 212 | 0.474 | 177 | 0.289 | Excellent |
| days) | | | | | (B) | | (B) | | |
| 122 (62 | 4.0 | 0.1 | 4.00 | 4.49 | 215 | 0.337 | 192 | 0.346 | Excellent |
| days) | | | | | (B) | | (B) | | |
| 124 (62 | 4.0 | 0.1 | 4.00 | 4.51 | 276 | 0.280 | 284 | 0.240 | Good |
| days) | | | | | (U) | | (B) | | |
| 125 (62 | 4.0 | 0.1 | 4.00 | 4.44 | 255 | 0.300 | 276 | 0.283 | Excellent |
| days) | | | | | (U) | | (U) | | |
| 126 (70 | 4.0 | 0.1 | 4.00 | 4.48 | 158 | 0.247 | 154 | 0.239 | Excellent |
| days) | | | | | (U) | | (U) | | |
| 127 (62 | 4.0 | 0.1 | 4.00 | 4.46 | 276 | 0.270 | 263 | 0.248 | Good |
| days) | | | | | (U) | | (B) | | |
| 128 (70 | 4.0 | 0.1 | 4.00 | 4.40 | 291 | 0.381 | 210 | 0.459 | Excellent |
| days) | | | | | (B) | | (B) | | |
| 130 (62 | 4.0 | 0.1 | 4.00 | 4.50 | 172 | 0.271 | 149 | 0.282 | Excellent |
| days) | | | | | (U) | | (U) | | |
| 133 (42 | 4.0 | 0.1 | 4.00 | 4.63 | 228 | 0.414 | 216 | 0.454 | Excellent |
| days) | | | | | (B) | | (B) | | |
| 135 (27 | 4.0 | 0.1 | 4.00 | 4.95 | 92 | 0.267 | 92 | 0.278 | Excellent |
| days) | | | | | (U) | | (U) | | |
| 136 (27 | 4.0 | 0.1 | 4.00 | 4.57 | 149 | 0.307 | 153 | 0.296 | Excellent |
| days) | | | | | (U) | | (U) | | |
| 138 (35 | 4.0 | 0.1 | 4.00 | 4.52 | 99 | 0.305 | 112 | 0.261 | Good |
| days) | | | | | (U) | | (B) | | |
| 141 (27 | 4.0 | 0.1 | 4.00 | 4.62 | 158 | 0.313 | 156 | 0.286 | Excellent |
| days) | | | | | (U) | | (U) | | |
| 142 (27 | 4.0 | 0.1 | 4.00 | 4.52 | 230 | 0.293 | 214 | 0.266 | Excellent |
| days) | | | | | (B) | | (U) | | |
| 143 (27 | 4.0 | 0.1 | 4.00 | 4.48 | 169 | 0.270 | 166 | 0.243 | Excellent |
| days) | | | | | (U) | | (U) | | |
| 144 (27 | 4.0 | 0.1 | 4.00 | 4.40 | 236 | 0.432 | 216 | 0.276 | Excellent |
| days) | | | | | (B) | | (U) | | |
| 145 (27 | 4.0 | 0.1 | 4.00 | 4.51 | 194 | 0.306 | 189 | 0.330 | Excellent |
| days) | | | | | (U) | | (U) | | |
| 146 (27 | 4.0 | 0.1 | 4.00 | 4.40 | 231 | 0.276 | 227 | 0.285 | Excellent |
| days) | | | | | (U) | | (U) | | |
| 147 (19 | 4.0 | 0.1 | 4.00 | 4.50 | 144 | 0.287 | 145 | 0.283 | Excellent |
| days) | | | | | (U) | | (U) | | |
| 148 (19 | 4.0 | 0.1 | 4.00 | 4.46 | 363 | 0.339 | 405 | 0.374 | Good |
| days) | | | | | (U) | | (B) | | |
| 149 (19 | 4.0 | 0.1 | 4.00 | 4.50 | 193 | 0.304 | 193 | 0.291 | Excellent |
| days) | | | | | (B) | | (B) | | |
| 150 (19 | 4.0 | 0.1 | 4.00 | 4.51 | 285 | 0.454 | 308 | 0.314 | Excellent |
| days) | | | | | (B) | | (U) | | |
| 151 (35 | 4.0 | 0.1 | 4.00 | 4.57 | 287 | 0.216 | 271 | 0.240 | Excellent |
| days) | | | | | (U) | | (U) | | |
| 152 (35 | 4.0 | 0.1 | 4.00 | 4.45 | 281 | 0.370 | 253 | 0.286 | Excellent |
| days) | | | | | (B) | | (U) | | |
| 153 (35 | 4.0 | 0.1 | 4.00 | 4.52 | 232 | 0.238 | 222 | 0.232 | Excellent |
| days) | | | | | (U) | | (U) | | |
| 154 (35 | 4.0 | 0.1 | 4.00 | 4.47 | 429 | 0.407 | 372 | 0.471 | Excellent |
| days) | | | | | (B) | | (B) | | |
| 161 (21 | 4.0 | 0.1 | 4.00 | 4.97 | 129 | 0.235 | 125 | 0.226 | Excellent |
| days) | | | | | (U) | | (U) | | |
| 162 (21 | 4.0 | 0.1 | 4.00 | 4.66 | 191 | 0.301 | 182 | 0.291 | Excellent |
| days) | | | | | (B) | | (B) | | |
| 171 (21 | 4.0 | 0.1 | 4.00 | 4.88 | 174 | 0.268 | 175 | 0.277 | Excellent |
| days) | | | | | (U) | | (U) | | |
| 172 (14 | 4.0 | 0.1 | 4.00 | 4.84 | 235 | 0.203 | 225 | 0.219 | Excellent |
| days) | | | | | (U) | | (U) | | |
| 173 (21 | 4.0 | 0.1 | 4.00 | 4.79 | 139 | 0.301 | 132 | 0.376 | Good |
| days) | | | | | (U) | | (B) | | |
| 177 (21 | 4.0 | 0.1 | 4.00 | 4.76 | 159 | 0.275 | 153 | 0.218 | Excellent |
| days) | | | | | (B) | | (B) | | |
| 178 (21 | 4.0 | 0.1 | 4.00 | 4.58 | 344 | 0.223 | 261 | 0.261 | Excellent |
| days) | | | | | (U) | | (U) | | |
| 110 (75 | 4.0 | 0.1 | 4.00 | 4.91 | 111 | 0.274 | 103 | 0.222 | Excellent |
| days) | | | | | (U) | | (U) | | |
| 111 (75 | 4.0 | 0.1 | 4.00 | 4.84 | 99 | 0.232 | 96 | 0.227 | Excellent |
| days) | | | | | (U) | | (U) | | |
| 155 (19 | 4.0 | 0.1 | 4.00 | 4.93 | 83 | 0.279 | 86 | 0.259 | Excellent |
| days) | | | | | (U) | | (U) | | |
| 156 (19 | 4.0 | 0.1 | 4.00 | 4.82 | 75 | 0.236 | 78 | 0.242 | Excellent |
| days) | | | | | (U) | | (U) | | |
| 157 (19 | 4.0 | 0.1 | 4.00 | 4.84 | 68 | 0.238 | 71 | 0.255 | Excellent |
| days) | | | | | (U) | | (U) | | |
| 158 (19 | 4.0 | 0.1 | 4.00 | 4.83 | 75 | 0.246 | 77 | 0.258 | Excellent |
| days) | | | | | (U) | | (U) | | |
| 159 (11 | 4.0 | 0.1 | 4.00 | 4.72 | 91 | 0.218 | 93 | 0.220 | Excellent |
| days) | | | | | (U) | | (U) | | |

a. All samples were prepared by injection in acetate buffer.
^{b}U: DLS curve showed unimodal size distribution; B: DLS curve showed bimodal size distribution; M: DLS curve showed multimodal size distribution.
^{c}Ranking of stability: Excellent (highlighted in green color) - the original sizes and distribution types remained almost the same after storage; Good - the original sizes and distribution types changed slightly (size difference was less than 100 nm) or the original size remained almost the same but the DLS curve showed more size distributions after storage.

### 9. Serum Stability of DNPs

(1) DNPs assembled from IAJD125 and IAJD155

Sample preparation: injected 25 μL ethanol stock solution of IAJD125/155 (80 mg/mL) into the mixture of 462.5 μL acetate buffer (10 mM, pH 4.0) and 12.5 μL Luc-mRNA (4.0 mg/mL in water), followed by vortex for 5 seconds.

Final composition of DNP solution: IAJD125/155 (4.0 mg/mL)+Luc-mRNA (0.1 mg/mL) in 500 μL acetate buffer. The dimension of assemblies in solution was checked by DLS.

### (2) Mixture of DNPs and Fetal Bovine Serum

DNPs in 1% Serum: Serum (5.5 μL)+[500 μL solution of IAJD (4.0 mg/mL) and Luc-mRNA (0.1 mg/mL].

The Table 22 below shows the dimensions of DNPs assembled from IAJD125 in 1% fetal bovine serum.

TABLE 22

| Samples | Size (nm) | PDI |
|---|---|---|
| DNPs of IAJD125 in acetate buffer (pH 4.0) | 196 | 0.273 |
| DNPs of IAJD125 in 1% Serum (t = 0 min) | 207 | 0.234 |
| DNPs of IAJD125 in 1% Serum (t = 10 min) | 206 | 0.290 |
| DNPs of IAJD125 in 1% Serum (t = 20 min) | 199 | 0.303 |
| DNPs of IAJD125 in 1% Serum (t = 30 min) | 197 | 0.256 |
| DNPs of IAJD125 in 1% Serum (t = 40 min) | 200 | 0.284 |
| DNPs of IAJD125 in 1% Serum (t = 50 min) | 190 | 0.339 |
| DNPs of IAJD125 in 1% Serum (t = 60 min) | 194 | 0.258 |

FIG. 76 shows dimensions of DNPs assembled from IAJD125 in 1% fetal bovine serum.

The Table 23 below shows the dimensions of DNPs assembled from IAJD155 in 1% fetal bovine serum.

TABLE 23

| Samples | Size (nm) | PDI |
|---|---|---|
| DNPs of IAJD155 in acetate buffer (pH 4.0) | 83 | 0.256 |
| DNPs of IAJD155 in 1% Serum (t = 0 min) | 93 | 0.186 |
| DNPs of IAJD155 in 1% Serum (t = 10 min) | 101 | 0.171 |
| DNPs of IAJD155 in 1% Serum (t = 20 min) | 105 | 0.186 |
| DNPs of IAJD155 in 1% Serum (t = 30 min) | 107 | 0.173 |
| DNPs of IAJD155 in 1% Serum (t = 40 min) | 108 | 0.161 |
| DNPs of IAJD155 in 1% Serum (t = 50 min) | 111 | 0.174 |
| DNPs of IAJD155 in 1% Serum (t = 60 min) | 108 | 0.149 |

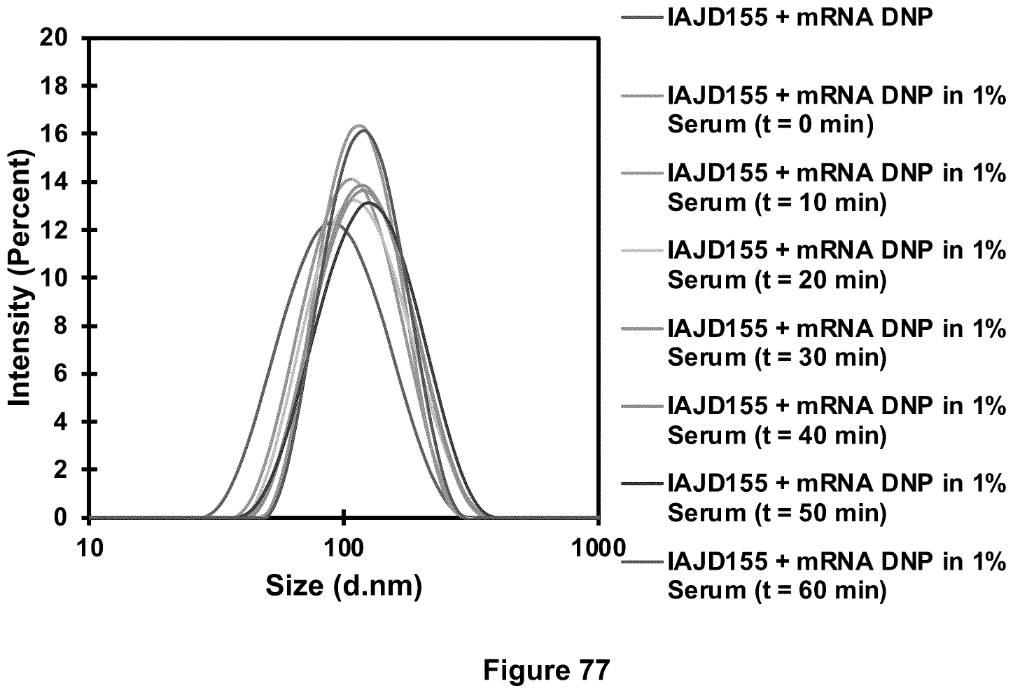

FIG. 77 shows dimensions of DNPs assembled from IAJD155 in 1% fetal bovine serum.

TABLE 24
pKa Measurements of
Individual IAJD Molecules

| IAJD No. | PKₐ |
|---|---|
| IAJD 113 | 6.52 |
| IAJD 114 | 6.43 |
| IAJD 115 | 6.32 |
| IAJD 116 | 6.51 |
| IAJD 117 | 6.49 |
| IAJD 118 | 6.33 |
| IAJD 119 | 6.44 |
| IAJD 120 | 6.37 |
| IAJD 122 | 6.66 |
| IAJD 124 | 6.65 |
| IAJD 125 | 6.64 |
| IAJD 126 | 6.52 |
| IAJD 127 | 6.52 |
| IAJD 128 | 6.68 |
| IAJD 130 | 6.62 |
| IAJD 133 | 6.57 |
| IAJD 135 | 6.32 |
| IAJD 136 | 6.50 |
| IAJD 138 | 6.62 |
| IAJD 141 | 6.56 |
| IAJD 142 | 6.69 |
| IAJD 143 | 6.33 |
| IAJD 144 | 6.51 |
| IAJD 145 | 6.28 |
| IAJD 146 | 6.59 |
| IAJD 147 | 6.32 |
| IAJD 148 | 6.65 |
| IAJD 149 | 6.50 |
| IAJD 150 | 6.65 |
| IAJD 151 | 6.39 |
| IAJD 152 | 6.65 |
| IAJD 153 | 6.42 |
| IAJD 154 | 6.48 |
| IAJD 161 | 6.38 |
| IAJD 162 | 6.33 |
| IAJD 171 | 6.16 |
| IAJD 172 | 6.03 |
| IJAD 173 | 5.91 |
| IAJD 177 | 5.90 |
| IAJD 178 | 5.97 |
| IAJD 110 | 7.64 |
| IAJD 111 | 7.58 |
| IAJD 155 | 7.40 |
| IAJD 156 | 7.43 |
| IAJD 157 | 7.42 |
| IAJD 158 | 7.38 |
| IAJD 159 | 7.40 |

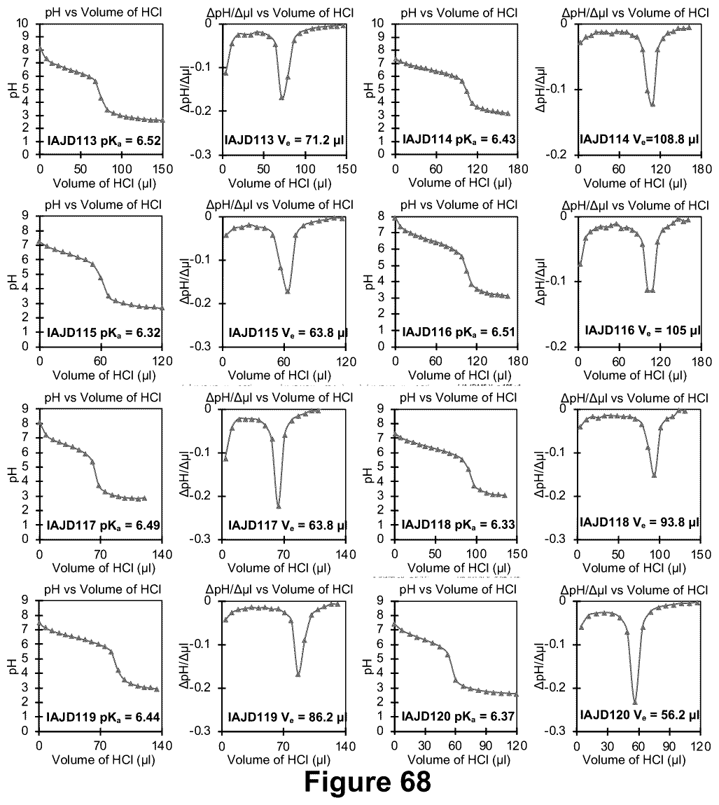

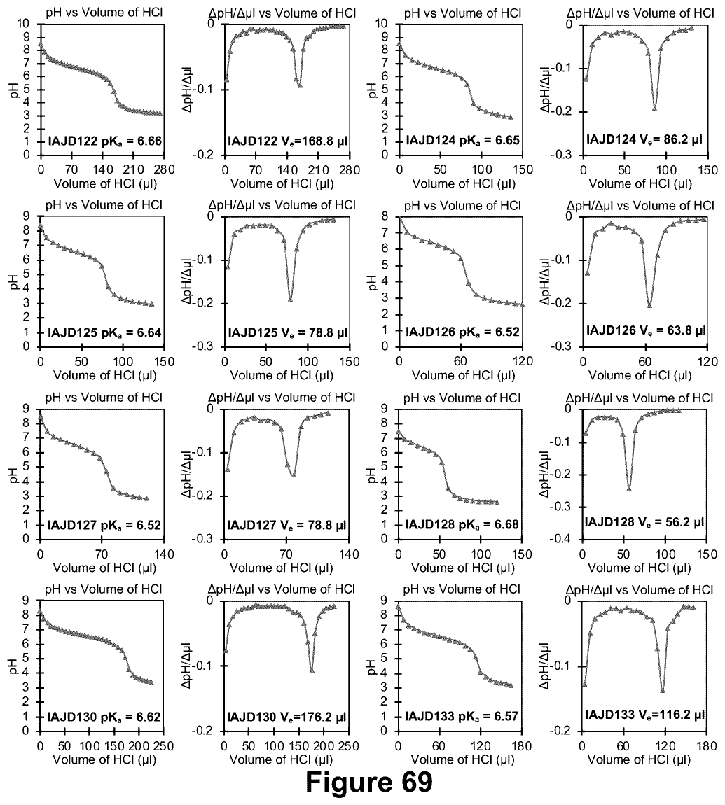

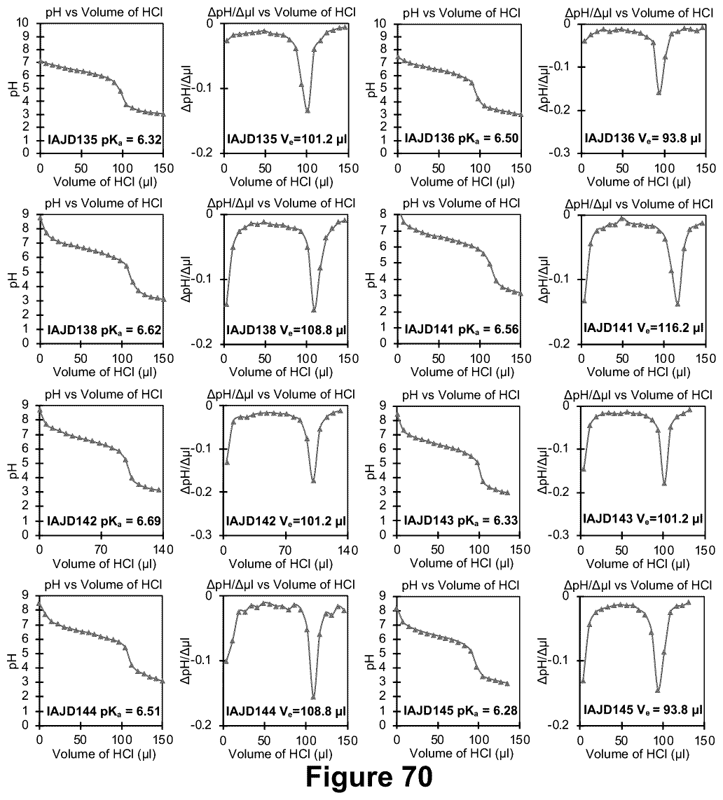

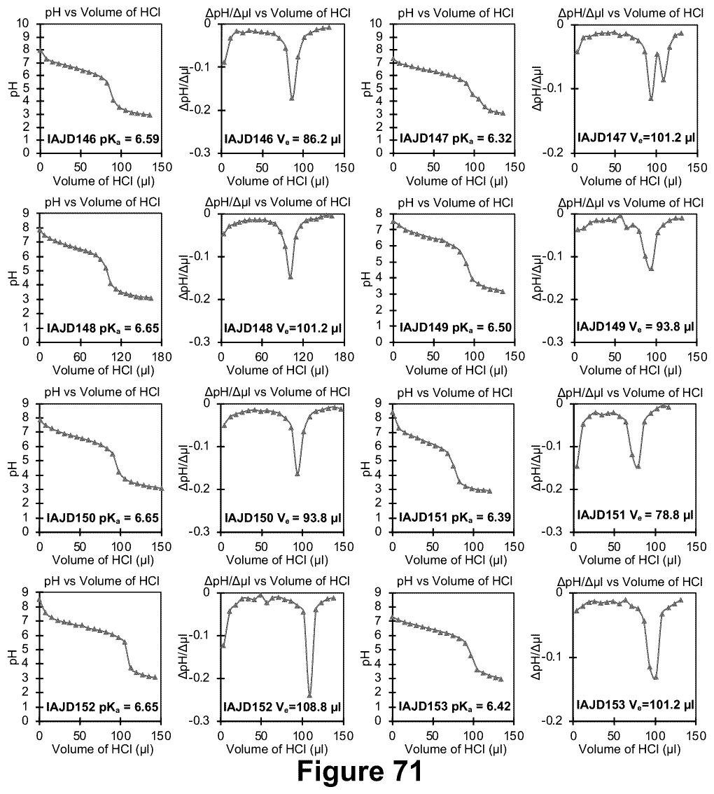

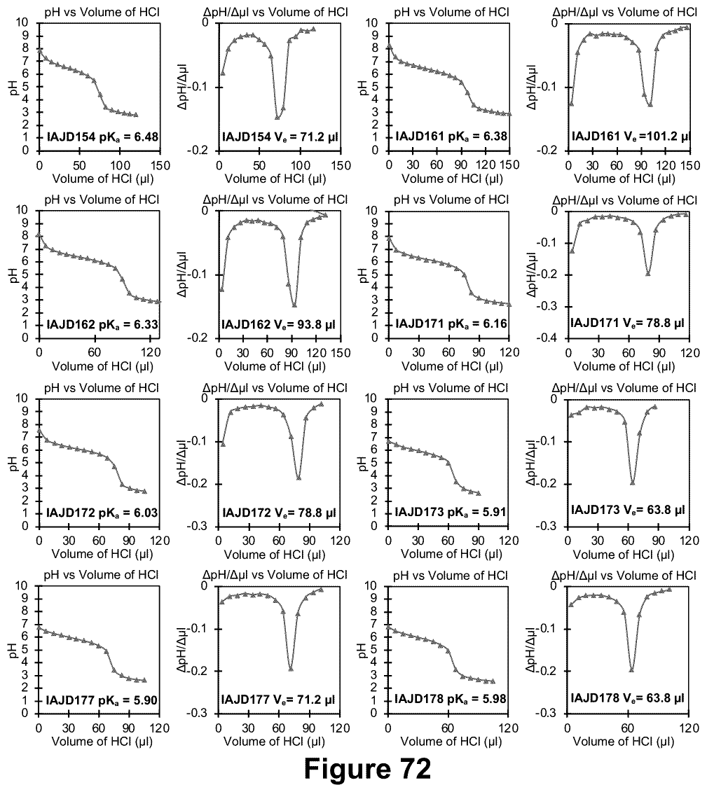

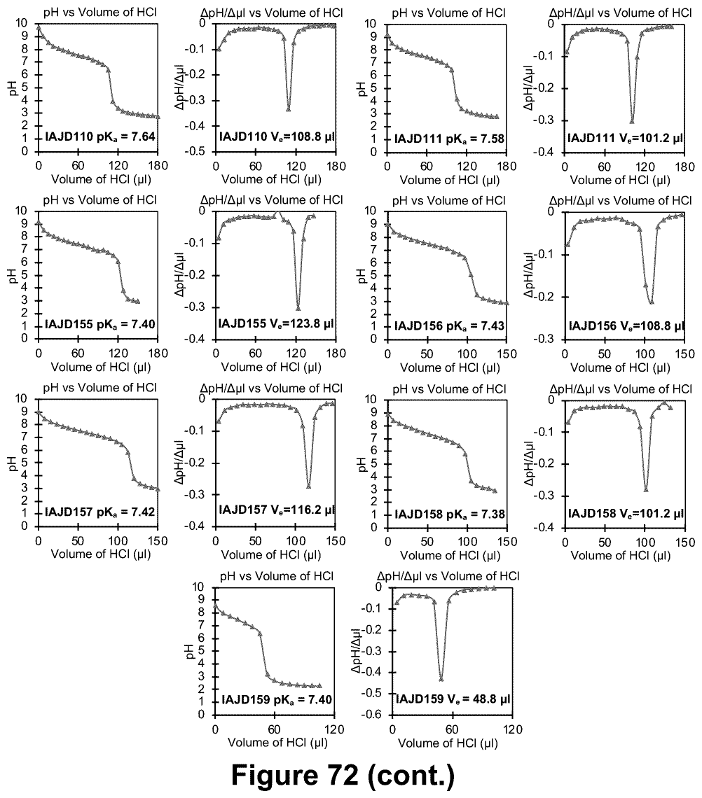

The titration curves and calculated pKₐ for IAJDs 110, 111, 113-120, 122, 124-128, 130, 133, 135, 136, 138, 141-159, are shown in FIG. 68 through FIG. 72.

### DLS Data of DNPs After Dialysis in PBS

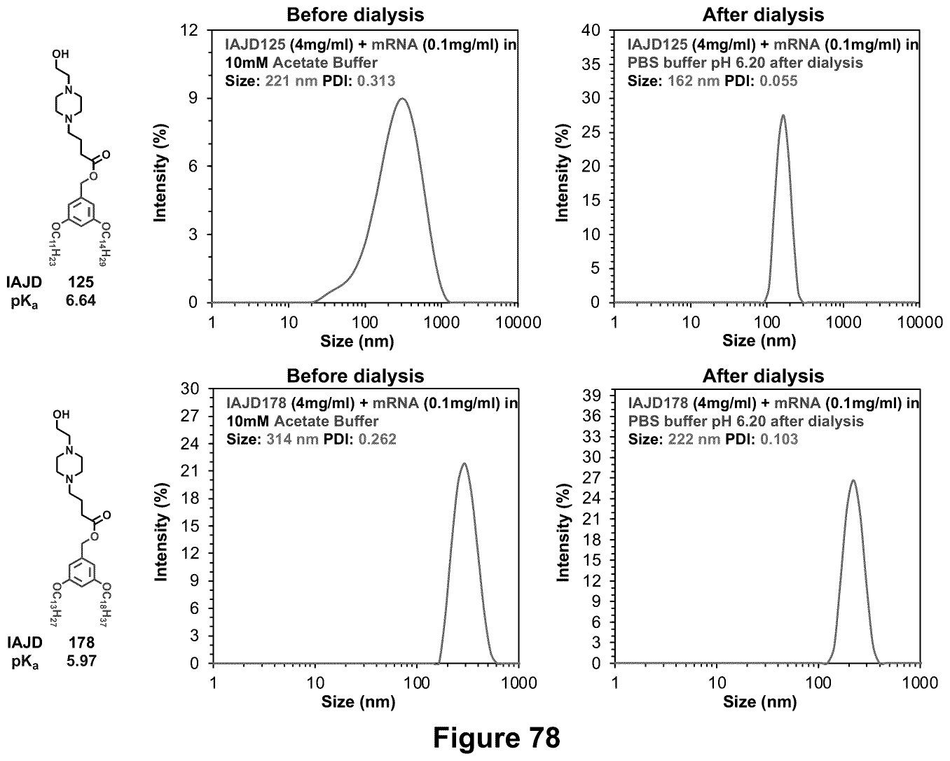

The 0.5 mL DNP solution (in acetate buffer) was dialyzed against 1000 mL 1×PBS buffer (pH 6.2) at 23° C. for 3 h in a dialysis tube with molecular weight cut-off of 3,500-14,000 (Spectrum Medical Instruments Inc. Spectra/Por molecular porous membrane tubing; Flat Width: 45 mm; Diameter: 29 mm & Vol/length: 6.4 mL/cm). FIG. 78 shows the DLS data of DNP 125 (a) and 178 (b) before and after dialysis in 1×PBS buffer for 3 h.

### Example 4: Self-Assembly of Glycerol-Amphiphilic Janus Dendrimers Indicates Principles for the Selection of Stereochemistry by Biological Membranes

Homochirality is an essential signature of living matter. During the evolution of life, nature selected homochirality for most biological molecules and macromolecules (Franck et al., On the One Hand but Not the Other: The Challenge of the Origin and Survival of Homochirality in Prebiotic Chemistry. In Chemistry for the 21th Century; Wiley-VCH: Weinheim, 2000; pp 175-208; Lehn et al., Chem. Soc. Rev. 2007, 36, 151-160; Hein et al., Top. Curr. Chem. 2013, 333, 83-108; Rowan et al., Angew. Chem. Int. Ed. 1998, 37, 63-68; Weissbuch et al., Chem. Rev. 2011, 111, 3236-3267). L-amino acids and D-sugars have been selected as the main components of biological systems (Kumar et al., Proc. Natl. Acad. Sci. U.S.A 2017, 114, 2474-2478; Yeom et al., Adv. Mater. 2020, 32, 1903878). However, phospholipids, the backbone of cell and endosome membranes, display different chirality in different living systems (Sojo et al., Orig. Life. Evol. Biosph. 2015, 45, 219-224; Liu et al., Nat. Chem. 2020, 12, 1029-1034). All natural phospholipids are homochiral. Nevertheless, a case of dual homochirality is also known for phospholipids. Archaea and bacteria, which are the two basal domains of life, display different homochirality in their phospholipids (Sojo et al., Orig. Life. Evol. Biosph. 2015, 45, 219-224; Liu et al., Nat. Chem. 2020, 12, 1029-1034; Coleman et al., Genome Biol. Evol. 2011, 3, 883-898). Archaea are based on sn-glycerol-i-phosphate (GIP) enantiomer, while bacteria employ exclusively sn-glycerol-3-phosphate (G3P) enantiomer (Koga et al., J. Mol. Evol. 1998, 46, 54-63). GIP and G3P exhibit opposite handedness, but both of them provide efficient membranes. A possible explanation is that the enzyme producing GIP existed simultaneously with that producing G3P but they evolved independently after the divergence of archaea and bacteria (Sojo et al., Orig. Life. Evol. Biosph. 2015, 45, 219-224; Liu et al., Nat. Chem. 2020, 12, 1029-1034).

Vesicles, an important class of biological assemblies, represent the basis of cell membranes. Synthetic variants, including liposomes (Bangham et al., J. Mol. Biol. 1964, 8, 660-668; Bangham et al., J. Mol. Biol. 1965, 13, 238-252; Seoane et al., J. Am. Chem. Soc. 2018, 140, 8388-8391; Ringsdorf et al., Angew. Chem. Int. Ed. Engl. 1988, 27, 113-158; Kunitake et al., Angew. Chem. Int. Ed. Engl. 1992, 31, 709-726; Brea et al., Chem. Eur. J. 2015, 21, 12564-12570; Flores et al., Chem. Soc. Rev. 2020, 49, 4602-4614; Bangham et al., J. Mol. Biol. 1964, 8, 660-668; Bangham et al., J. Mol. Biol. 1965, 13, 238-252; Seoane et al., J. Am. Chem. Soc. 2018, 140, 8388-8391; Ringsdorf et al., Angew. Chem. Int. Ed. Engl. 1988, 27, 113-158; Kunitake et al., Angew. Chem. Int. Ed. Engl. 1992, 31, 709-726; Brea et al., Chem. Eur. J. 2015, 21, 12564-12570; Flores et al., Chem. Soc. Rev. 2020, 49, 4602-4614), stealth liposomes (Lasic et al., Chem. Rev. 1995, 95, 2601-2628: Immordino et al., Int. J. Nanomedicine 2006, 1, 297-315), and polymersomes (Discher et al., Science 1999, 284, 1143-1146; Lee et al., J. Controlled Release 2012, 161, 473-483; Deming et al., WIREs Nanomed. Nanobiotechnol. 2014, 6, 283-297) have been elaborated to mimic natural cells. In addition to being models for biological membranes synthetic vesicles have been used to deliver active agents such as drugs and genes in vitro and in vivo (Allen et al., Science 2004, 303, 1818-1822; Torchilin et al., Nat. Rev. Drug Discovery 2005, 4, 145-160; de Jong et al., Acc. Chem. Res. 2019, 52, 1761-1770; EL Andaloussi et al., Nat. Rev. Drug. Discov. 2013, 12, 347-357; Dong et al., J. Am. Chem. Soc. 2018, 140, 16264-16274; Lee et al., Nat. Biotechnol. 2005, 23, 1517-1526; Tu et al., Chem. Rev. 2016, 116, 2023-2078; Thota et al., Chem. Rev. 2016, 116, 2079-2102; Weiss et al., Nat. Mater. 2018, 17, 89-96; Gillies et al., Drug Discov. Today 2005, 10, 35-43; Dendrimers and Other Dendritic Polymers; Tomalia, D. A.; Fréchet, J. M. J., Eds.; Wiley. Amsterdam, The Netherlands, 2001). Liposomes assembled from phospholipids are unstable because the alkene units in the hydrophobic tails are readily oxidized and exhibit poor mechanical properties. Stealth liposomes co-assembled from phospholipids, phospholipid conjugated with poly(ethylene glycol) (PEG) and cholesterol are more stable, but are polydisperse requiring time-consuming fractionation to desirable dimensions and narrow polydispersity. Polymersomes assembled from amphiphilic block copolymers exhibit excellent stability but are not always biocompatible and their membrane thickness is wider than that of cell membranes (Discher et al., Science 1999, 284, 1143-1146; Lee et al., J. Controlled Release 2012, 161, 473-483; Deming et al., WIREs Nanomed Nanobiotechnol. 2014, 6, 283-297).

The previous studies have developed a class of vesicles named dendrimersomes (DSs), which are assembled from amphiphilic JDs (Percec et al., Science 2010, 328, 1009-1014; Percec et al., J. Am. Chem. Soc. 2013, 135, 9055-9077; Xiao et al., J. Am. Chem. Soc. 2016, 138, 12655-12663; Rodriguez-Emmenegger et al., Proc. Natl. Acad Sci. U.S.A 2019, 116, 5376-5382; Sherman et al., Chem. Rev. 2017, 117, 6538-6631; Peterca et al., J. Am. Chem. Soc. 2011, 133, 20507-20520). DSs exhibit better stability than liposomes and can be functionalized on their surface through their multivalency both in the hydrophilic and hydrophobic parts of JDs (Torre et al., Proc. Natl. Acad Sci. U.S.A. 2019, 116, 15378-15385; Xiao et al., Proc. Natl. Acad Sci. U.S.A. 2020, 117, 11931-11939; L¹ et al., ACS Nano 2020, 14, 7398-7411). Amphiphilic JDs with sugars conjugated on their hydrophilic part denoted Janus glycodendrimers (JGDs) self-assemble into glycodendrimersomes (GDSs), which mimic the glycan of biological membranes and bind sugar-binding proteins (Xiao et al., Proc. Natl. Acad Sci. U. S. A. 2018, 115, E2509-E2518). DSs are assembled by injection of their JD in water-miscible solvent solution such as ethanol or THF into water or buffer. This simple assembling procedure provides monodisperse, impermeable and stable vesicles with excellent mechanical properties.

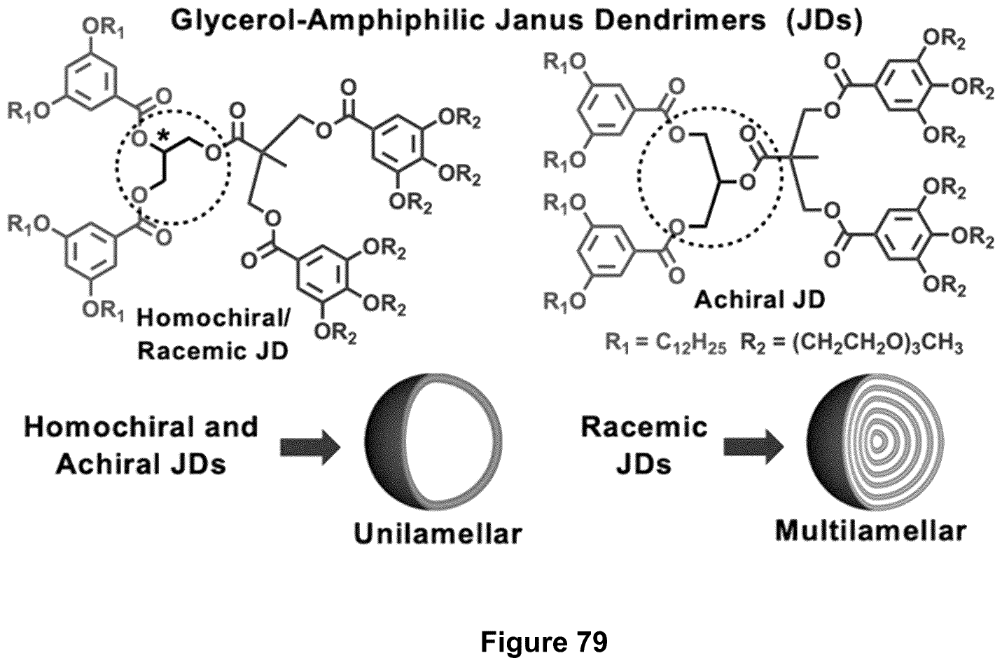

The properties of DSs rely on the architecture of the assembling amphiphilic JDs. JDs assembling DSs were classified into “Twin-Twin” and “Single-Single”. “Twin-Twin” JDs are constructed by combinations of twin-hydrophobic and twin-hydrophilic dendrons with an achiral pentaerythritol (PE) or tris(hydroxymethyl)aminomethane (Tris) core, while “Single-Single” JDs are made from single-hydrophobic and single-hydrophilic dendrons connected by an ester or amide linker. Unilamellar and onion-like DSs have been discovered, designed and predicted (Peterca et al., J. Am. Chem. Soc. 2011, 133, 20507-20520) by self-assembly of “Twin-Twin” and “Single-Single” JDs (Zhang et al., Proc. Natl. Acad Sci. U.S.A 2014, 111, 9058-9063; Zhang et al., ACS Nano 2014, 8, 1554-1565; Zhang et al., J. Am. Chem. Soc. 2015, 137, 13334-13344; Xiao et al., Proc. Natl. Acad. Sci. U.S.A 2016, 113, 1162-1167; Kostina et al., Soft Matter 2020, DOI: 10.1039/D0SM01097A). However, there is no report on amphiphilic JDs with a chiral or achiral glycerol core connecting hydrophilic and hydrophobic parts (FIG. 79). Nature selects homochiral rather than racemic but not achiral glycerol (FIG. 80) phospholipids to construct biological membranes.

The present studies investigated the self-assembly of all constitutional isomeric glycerol-JDs that facilitate the construction of homochiral, racemic and achiral structures via the stereochemistry of the branching point. Eight JDs including four homochiral, two racemic and two achiral were synthesized (FIG. 80). Injection of their ethanol solution into water produced DSs with different dimensions and morphologies. They were characterized by dynamic light scattering (DLS) and cryogenic-transmission electron microscopy (cryo-TEM). Statistical analysis of the number of bilayers showed that homochiral JDs self-assemble predominantly unilamellar, achiral only unilamellar, while racemic predominantly multilamellar, onion-like DSs. This result provides the rationale for the selection of homochirality of phospholipids by nature.

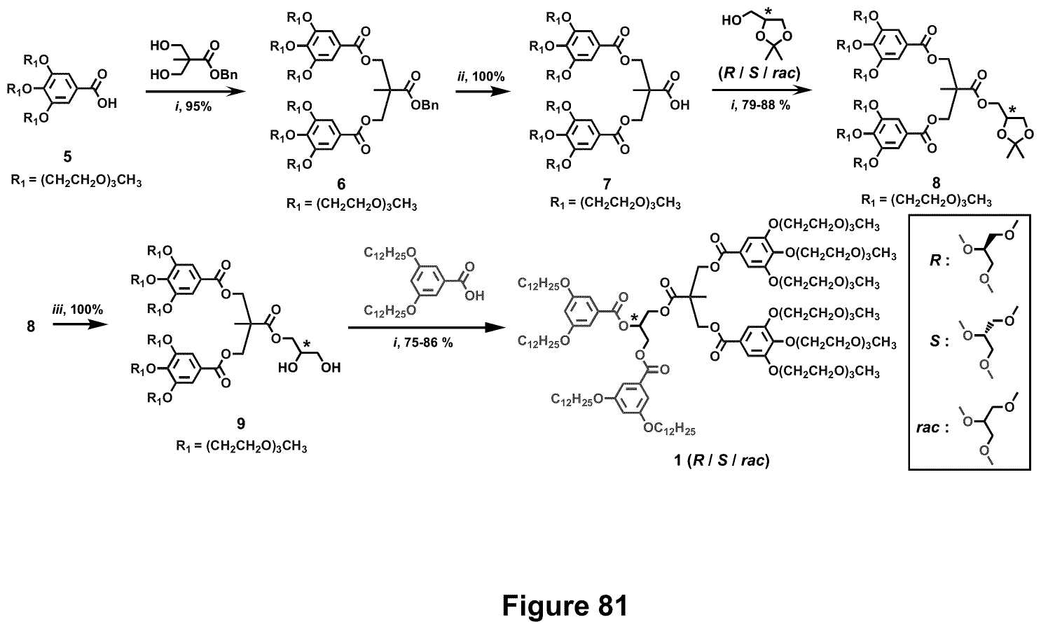

Three libraries of glycerol-based JDs were synthesized by an orthogonal-modular methodology. For library 1 and 2, each library has two JDs with homochiral glycerol branching point and one JD with racemic branching point (FIG. 80). Library 3 contains two constitutional isomeric glycerol-achiral JDs (FIG. 80). The main building blocks employed in the orthogonal-modular synthesis include the hydrophilic acid (5), hydrophilic second-generation acid (7), hydrophobic acid (10), hydrophobic second-generation acid (12), R/S rac-2,2-dimethyl-1,3-dioxolane-4-methanol (isopropylidene glycerol) which contains the stereocenter and 5-hydroxy-2-phenyl-1,3-dioxane (FIG. 81 through FIG. 83).

The hydrophilic acid (5) was synthesized by introducing triethylene glycol monomethyl ether in the 3, 4, 5 positions of gallic acid. Two hydrophilic acids (5) were connected by a 2,2-bis(hydroxylmethyl)propionate linker to afford the hydrophilic second-generation acid (7). Similarly, hydrophobic acid (10) was synthesized by attaching dodecyl groups on the 3, 5 positions of 3,5-dihydroxybenzoic acid. The hydrophobic second-generation acid (12) was prepared by connecting two hydrophobic acids (10) with a 2,2-bis(hydroxylmethyl)propionate linker. For library 1, hydrophilic second-generation acid (7) reacted with rac/R/S-2,2-dimethyl-1,3-dioxolane-4-methanol, followed by deprotection to afford the corresponding diol (9). The esterification of this hydrophilic diol and two hydrophobic first-generation acids produced 1R, 1S and 1rac in 75-86% yield.

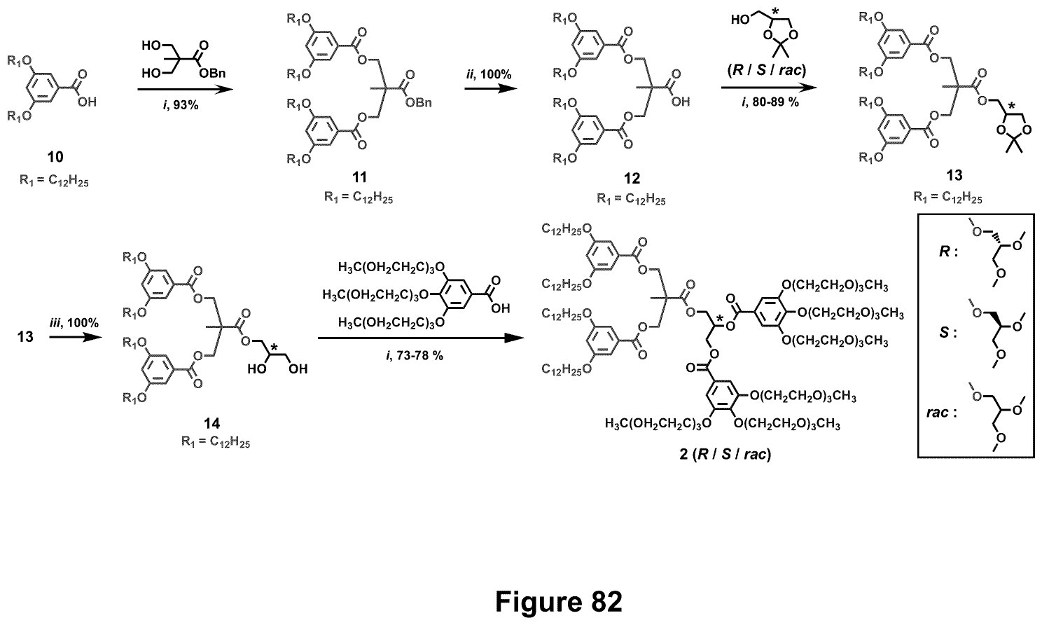

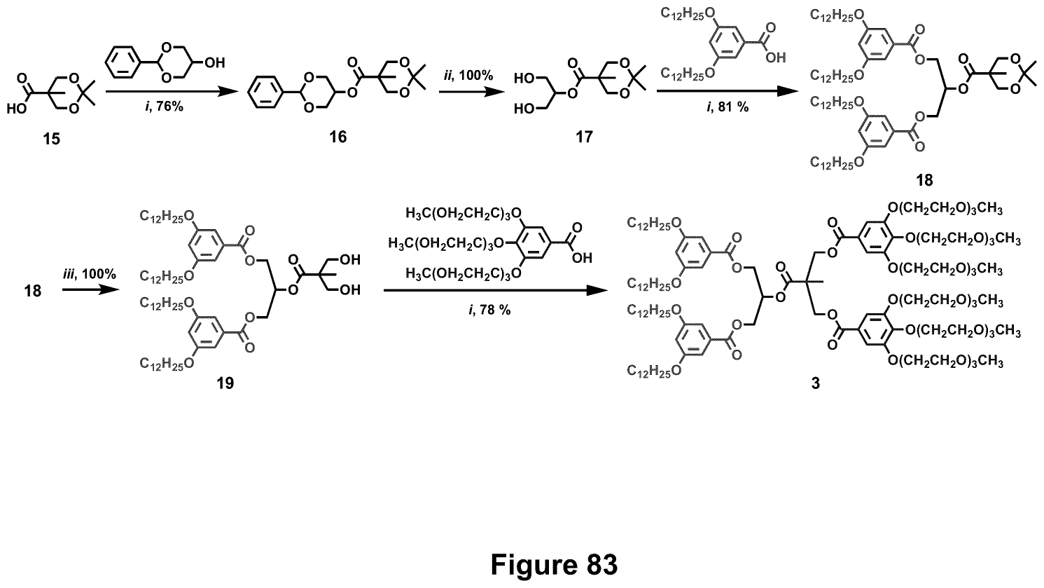

The synthesis of the three molecules of library 2 (2R, 2S and 2rac) (FIG. 82) was accomplished with the first esterification performed with hydrophobic second-generation acid and the second esterification with the hydrophobic diol (14) and two hydrophilic acids (5) in 73-78% yield. Detailed synthesis and characterization are in the supporting information. FIG. 83 and Scheme 21 outline the synthesis of the achiral JDs (3) and (4).

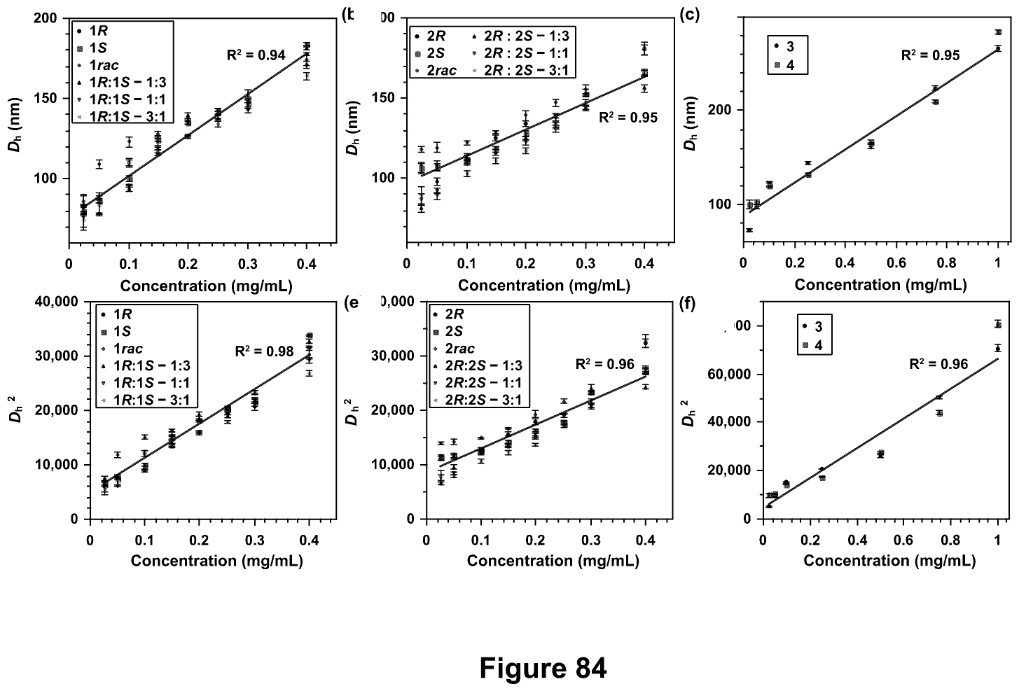

Assembly of all eight glycerol-JDs was performed by injection of their ethanol solution (50 μL) into 1 mL of Milli-Q water. The size distribution and structure of resulting DSs were analyzed by DLS (FIG. 84) and cryo-TEM. Previously, it was found that unilamellar and onion-like DSs assembled from “Twin-Twin” and “Single-Single” JDs displayed size-concentration dependence (Peterca et al., J. Am. Chem. Soc. 2011, 133, 20507-20520; Zhang et al., ACS Nano 2014, 8, 1554-1565). In a certain concentration range the size of DSs increased with the concentration of JDs injected in water or buffer. A similar size-concentration dependence was observed for glycerol-JDs. FIG. 84 shows how the size of the resulting DSs increased with the concentration of JDs in water for 1R, 1S and 1rac for concentration range from 0.025 to 0.5 mg mL-1. The dimensions of the assemblies were identical when the concentrations of the JDs in water were identical. Sizes of assemblies from mixtures of 1R and 1S in different ratios (1:3, 1:1 and 3:1), were also identical (FIG. 84). This indicated that sizes of assemblies from JD enantiomers (1R and 1S) and racemic mixtures with different ratios of enantiomers were identical at the same concentrations of JDs in water. With the exception of the rotation of polarized light, the physical properties of enantiomers and their racemic mixtures must be identical. In addition, the square of the diameter of DSs displayed a linear dependence on concentration in the range from 0.025 to 0.4, for homochiral and racemic, and to 1.0 mg mL-1 for achiral (FIG. 84). This result is consistent with the size-concentration relationship of JDs already reported (Peterca et al., J. Am. Chem. Soc. 2011, 133, 20507-20520; Zhang et al., ACS Nano 2014, 8, 1554-1565) and helps predict the dimensions of DSs with the aid of JDs concentration. 2R, 2S and 2rac as well as mixtures of 2R and 2S with different molar ratios (1:3, 1:1 and 3:1) showed similar size-concentration dependence (FIG. 84). The diameter rather than the square of diameter of DSs displayed a linear relationship versus concentration of glycerol-JD in the concentration range from 0.025 to 0.4 for homochiral and racemic and to 1.0 mg mL-¹ for achiral (FIG. 84).

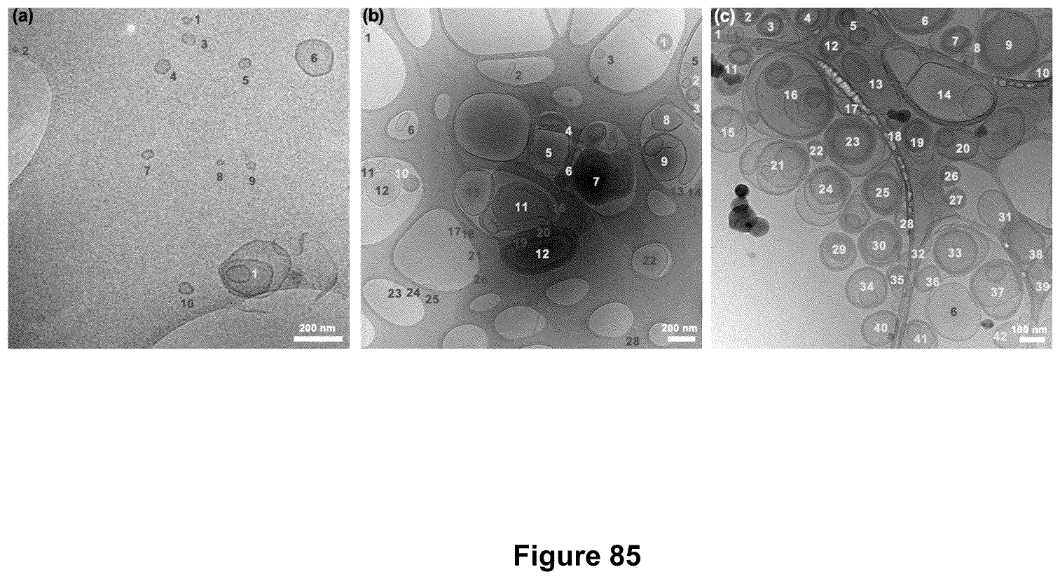

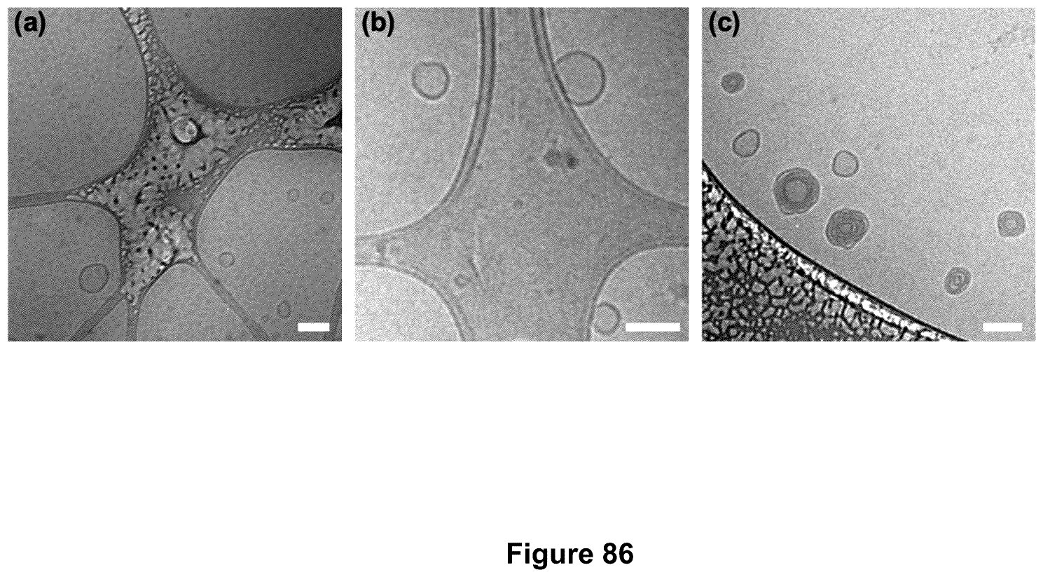

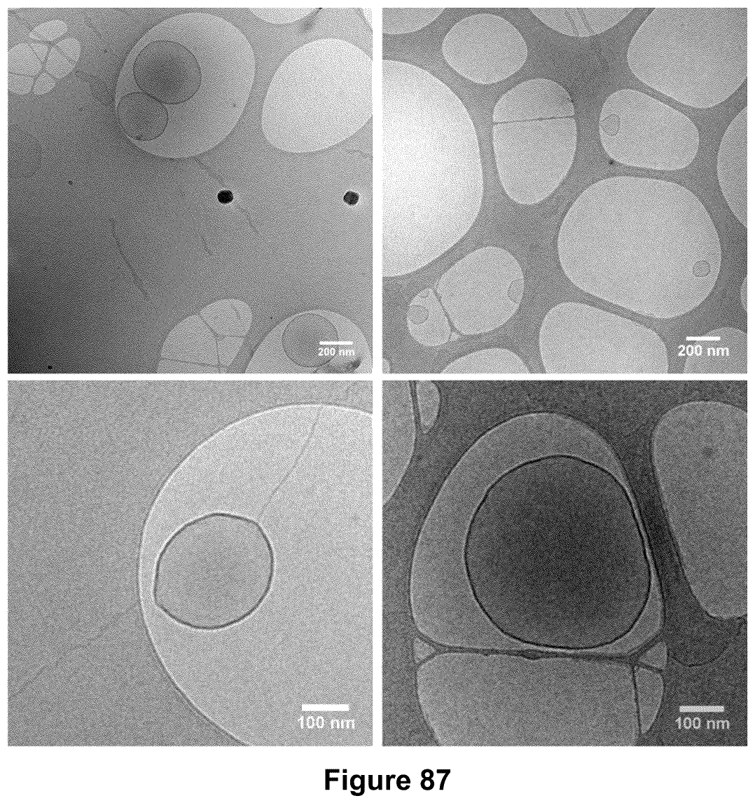

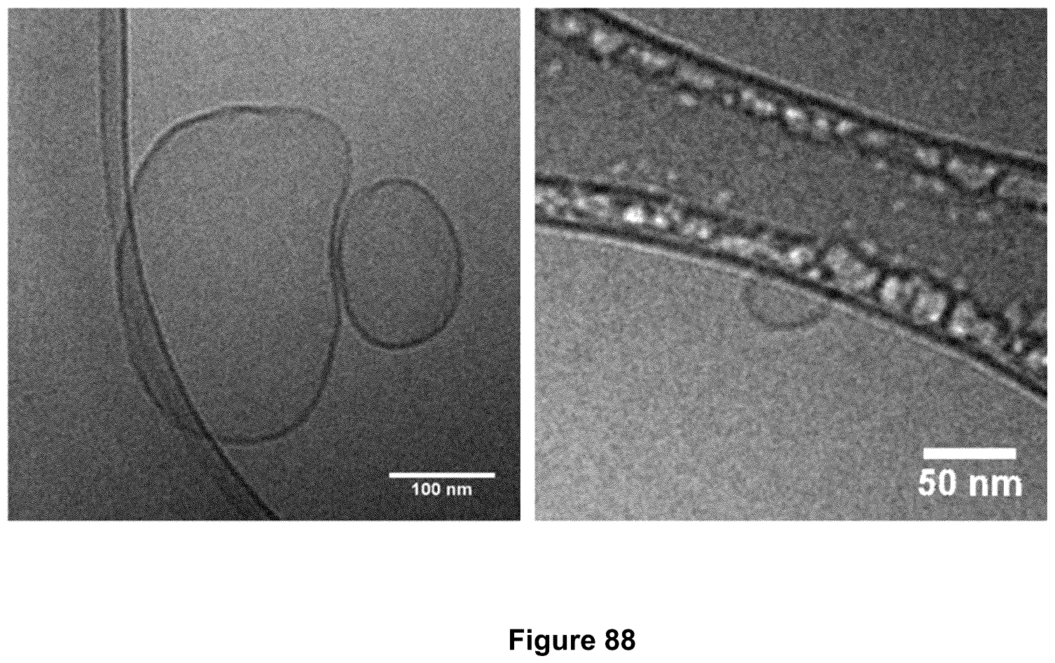

Although dimensions do not show differences when comparing homochiral, achiral and racemic JDs, the structure of the resulting DSs was investigated by cryo-TEM. Substantial differences were observed. cryo-TEM of enantiomers 2R and 2S (FIG. 85) showed unilamellar DSs predominantly, being accompanied by a small amount of multilamellar. cryo-TEM images of enantiomers 2rac (FIG. 85) showed the opposite result, in which multilamellar DSs were predominant while unilamellar were observed in small amount. The red numbers in FIG. 85 stand for the unilamellar while yellow numbers for multilamellar DSs. The calculated percentages of unilamellar DSs in the selected images for 2R, 2S and 2rac are 91%, 70% and 13%, respectively. Same trend was found for cryo-TEM of 1R, 1S and 1rac (FIG. 86 through FIG. 88). This indicates that homochiral JDs tend to self-assemble predominantly into unilamellar while racemic favor the formation of multilamellar DSs. Achiral DSs are all unilamellar.

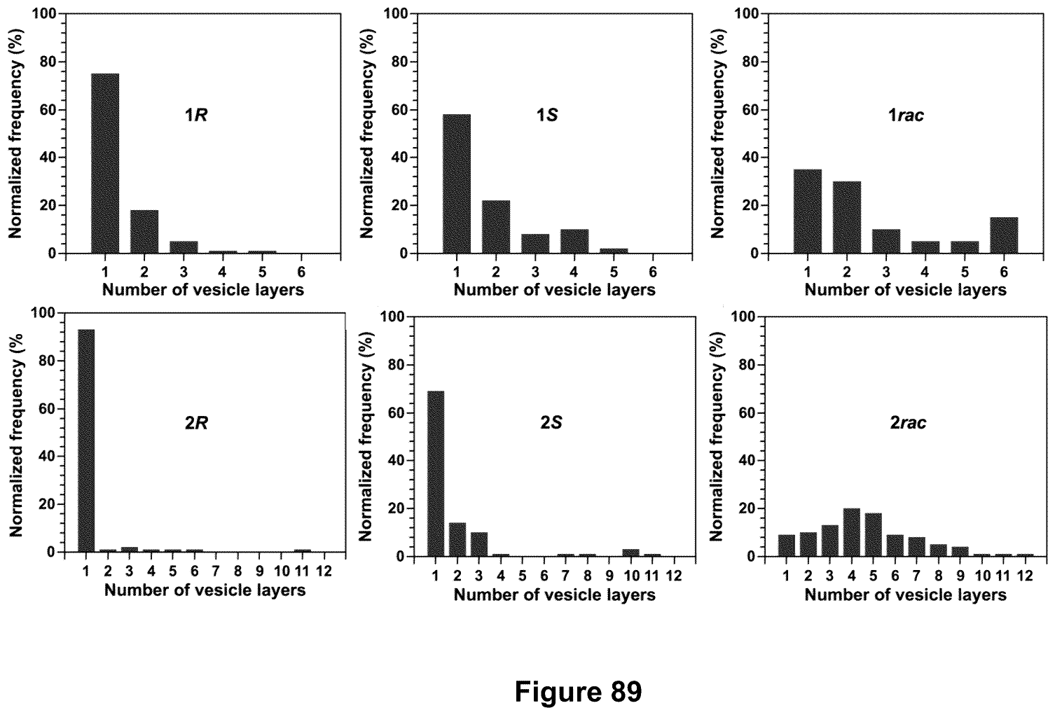

Statistical analysis was used to organize the number of bilayers from cryo-TEM. The distribution of the number of lamellae in DSs was analyzed for each sample to construct the respective histograms (FIG. 89). The histograms represent the distribution function of probability. The number of bilayers of racemic JDs (1rac and 2rac) exhibit a near normal distribution (Gaussian) as demonstrated by various statistic tests. However, the enantiomerically pure JDs (1R, 1S, 2R and 2S) exhibit chi-squared distribution (V distribution) with a shape parameter of 1 or 2. The difference in the distributions of number of bilayers for homochiral vs racemic glycerol-based JDs emphasizes the difference between the self-assembly structures, unilamellar vs multilamellar.

To investigate the normality of the distribution of racemic JDs, SPSS statistics software, which utilized normality tests of Kolmogorov-Smirnov and Shapiro-Wilk, was used. Both methods demonstrated that the distribution of racemic JDs (1rac and 2rac) were normal-like. For a more detailed study of distribution functions, the skewness (third standardized moment and a measure of the asymmetry) and kurtosis (fourth standardized moment and measure the extreme values in either tail) of the data were calculated (number of DSs layers). The skewness of the distributions of the number of bilayers for 1rac and 2rac is slightly positive (0.70 and 0.58, respectively). Positive skewness means the tails are extending to the right. These relatively low values are in line with the previous conclusion that their distribution is normal-like. Comparing the skewness of the distribution of racemic JDs with homochiral JDs shows that 1R, 1S, 2R and 2S are very positively skewed (1.977, 1.687, 3.313 and 3.029, respectively) and non-symmetric. This difference in distribution parameter (skewness) strongly supports the difference between homochiral (unilamellar) and racemic (multilamellar) glycerol-based JDs.

The second distribution parameter, which was investigated, is the kurtosis and excess kurtosis (kurtosis—3). For normal distribution the kurtosis is 3 and the excess of kurtosis is zero. The distributions of racemic JDs have negative Kurtosis (−1.623 for 1rac and −0.423 for 2rac), indicating that the peak is lower than the peak of a normal distribution. While homochiral JDs show distributions with positive kurtosis (3.932 for 1R, 2.797 for 1S, 10.980 for 2R and 9.502 for 2S), indicating that corresponding peaks are higher than those of the normal distribution. This result highlights the difference between the number of bilayers of DSs assembled from homochiral and racemic glycerol-JDs.

Glycerol-JDs with homochiral, achiral and racemic branching points were synthesized. They self-assemble into monodisperse DSs by simple injection of their ethanol solution into water. DLS showed that the resulting DSs are concentration-size dependent with an identical trend regardless of achiral, homochiral or racemic branching point. Cryo-TEM and statistical analysis showed that homochiral JDs form predominantly unilamellar while racemic JDs favor multilamellar DSs. Achiral produce only unilamellar DSs. This study explains the selection of homochiral lipids but does not why achiral were eliminated, since single-bilayer membranes are required for the construction of biological cell membranes.

In conclusion, three libraries of constitutional isomeric glycerol-amphiphilic Janus dendrimers (JDs) with homochiral, racemic and achiral branching points were synthesized by an orthogonal-modular methodology. Monodisperse vesicles known as dendrimersomes (DSs) with predictable dimensions programmed by JD concentration were assembled by injection of their ethanol solution into water. DSs of JD enantiomers, racemic including mixtures of different enantiomers, and achiral exhibited identical concentration-size dependence. However, the number of bilayers of DSs from homochiral, achiral and racemic JDs determined by cryo-TEM were different. Statistical analysis of the number of bilayers demonstrated that homochiral and achiral (JDs) formed predominantly or only unilamellar while racemic (JDs) favored multilamellar DSs. These results provide a rationale for homochiral vs racemic selection.

The materials and methods employed in these experiments are now described.

### Materials

(R)-2,2-dimethyl-1,3-dioxolane-4-methanol (TCI, 98%) and (S)-2,2-dimethyl-1,3-dioxolane-4-methanol (Alfa Aesar, 98%) and concentrated hydrochloric acid (Fisher, ACS grade) were used as received. 4-(Dimethylamino)pyridinium 4-toluenesulfonate (DPTS) was prepared according to a literature procedure (Moore et al., Macromolecules 1990, 23, 65-70). Benzyl 2,2-bis(hydroxymethyl)propionate was prepared according to a literature procedure (Córdova et al., J Am. Chem. Soc. 2001, 123, 8248-8259). CH₂Cl₂ (DCM) was dried over CaH₂ and freshly distilled before use. All other reagents and solvents were obtained from commercial sources and were used without purification unless otherwise stated.

### Methods

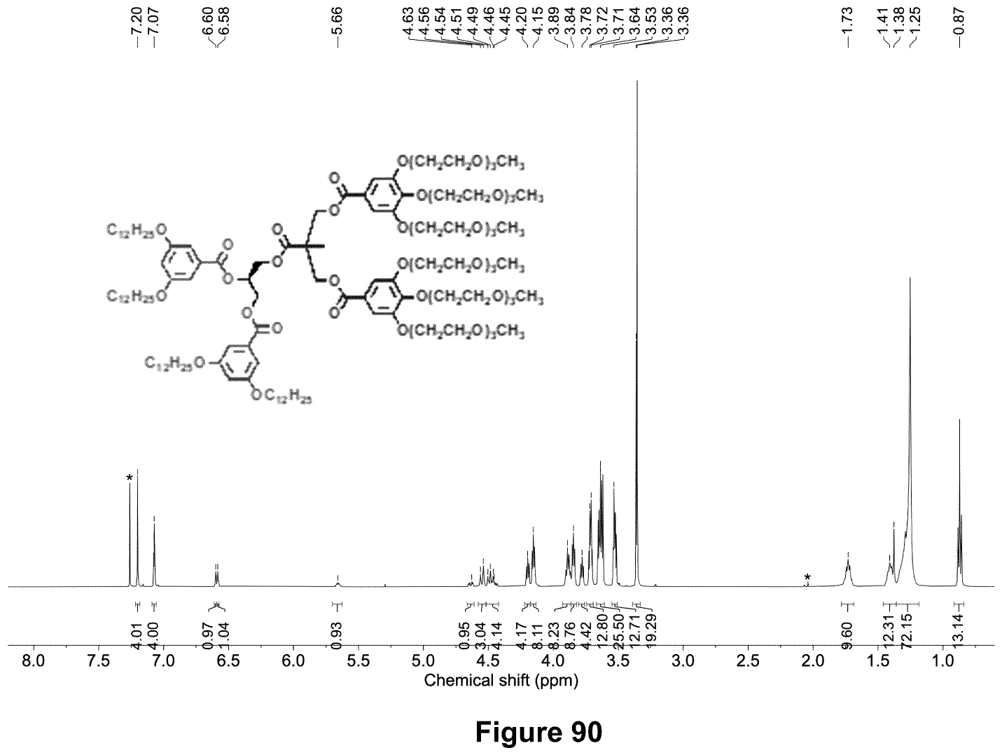

NMR. ¹H NMR and ¹³C NMR spectra were recorded on Bruker DRX (500 MHz) spectrometer at 298 K. Deuterated chloroform (CDCl₃) was used as solvent. The resonance multiplicities in the ¹H NMR spectra are described as “s” (singlet), “d” (doublet), “t” (triplet), and “m” (multiplet) and broad resonances are indicated by “br”. NMR chemical shifts were calculated with the residual protic solvent of CDCl₃ (¹H, δ 7.26 ppm; ¹³C, δ 77.16 ppm) as internal reference (FIG. 90 through FIG. 105).

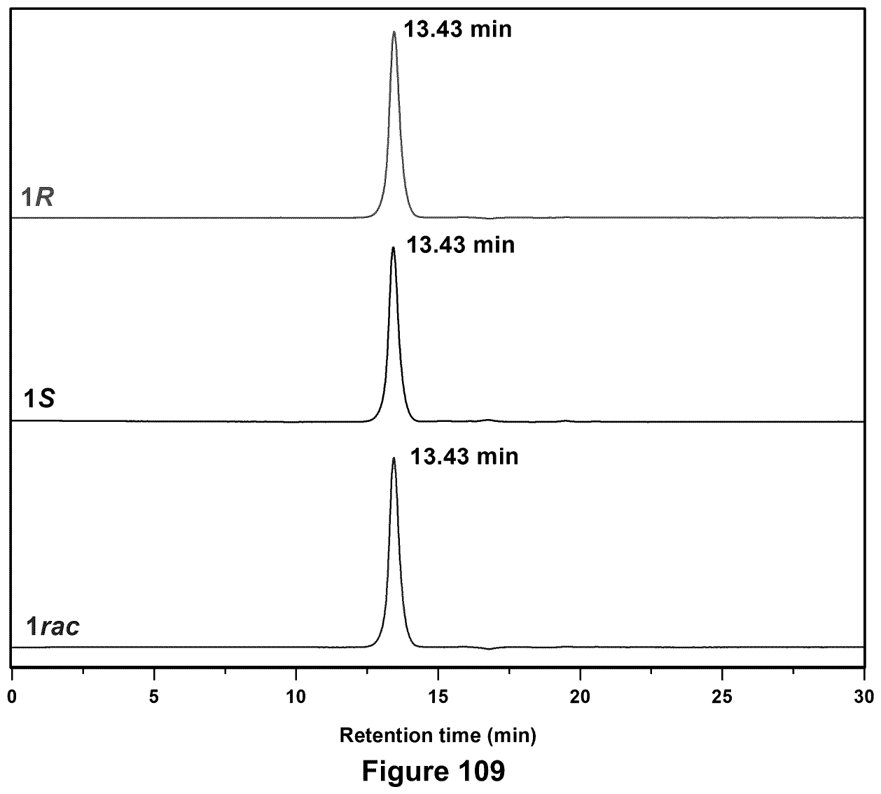

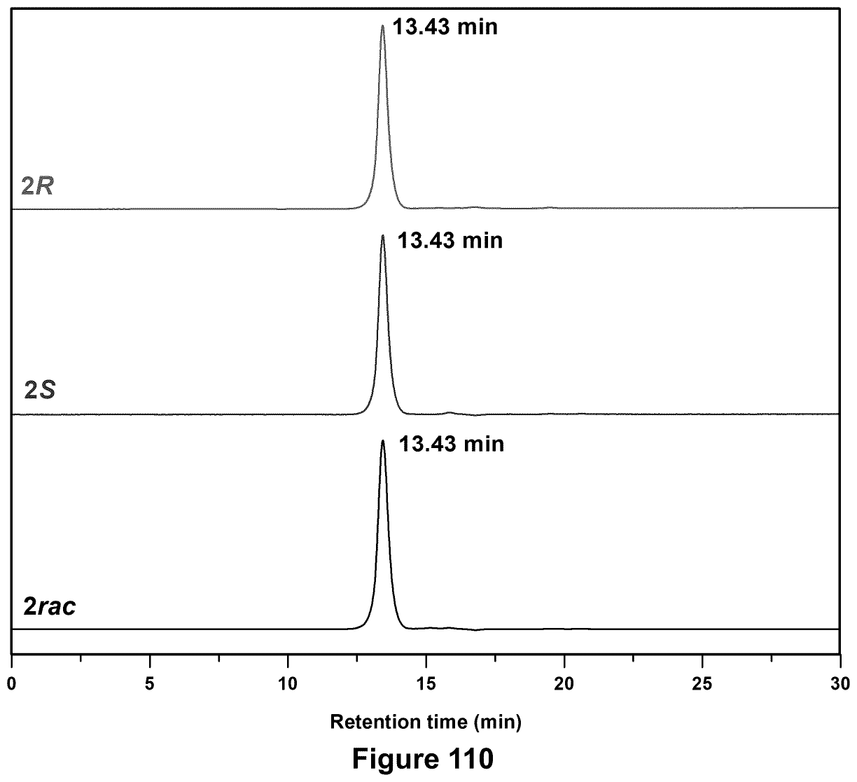

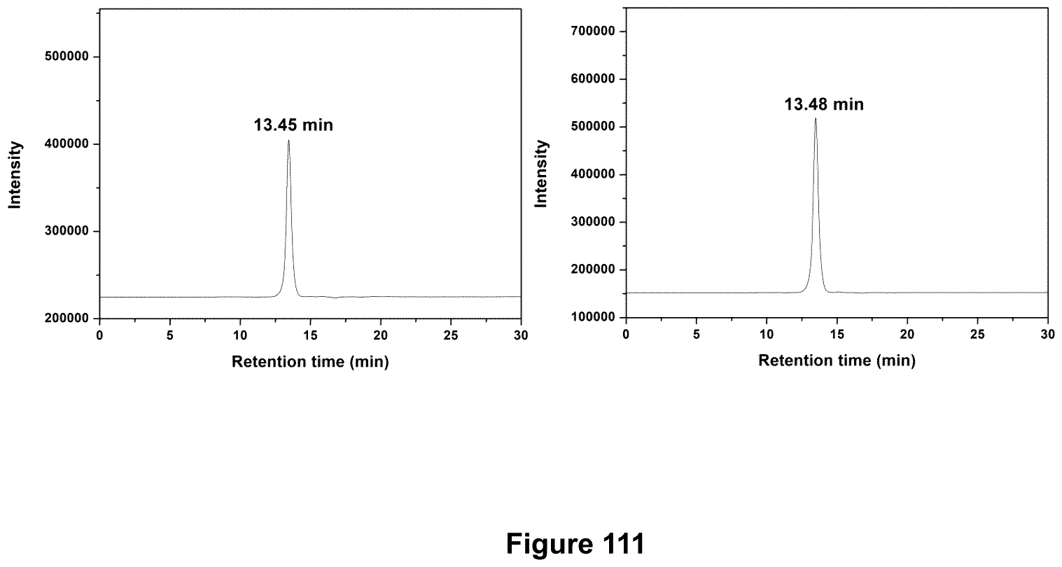

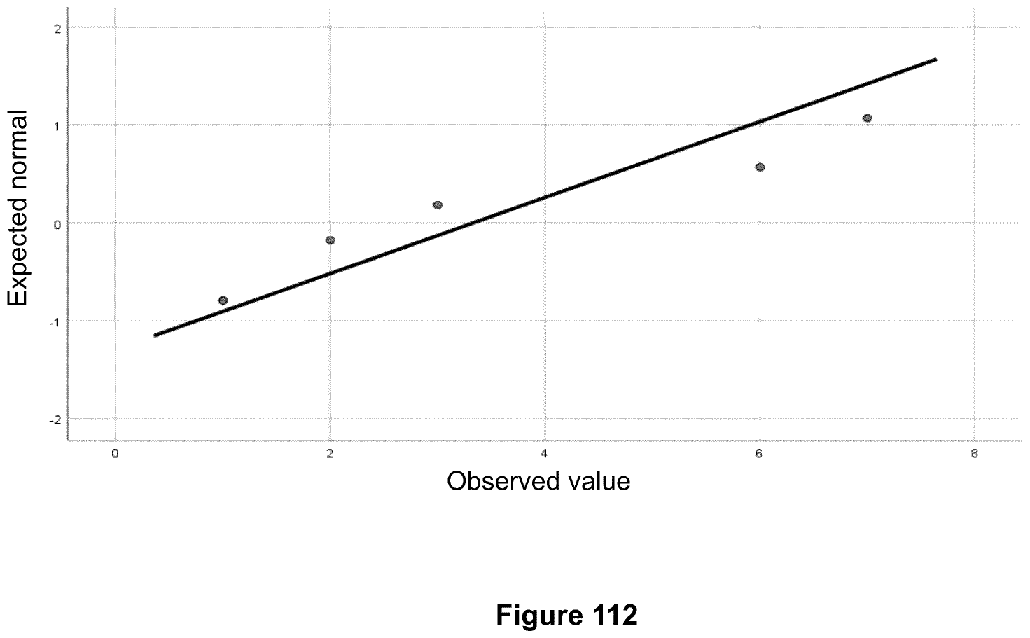

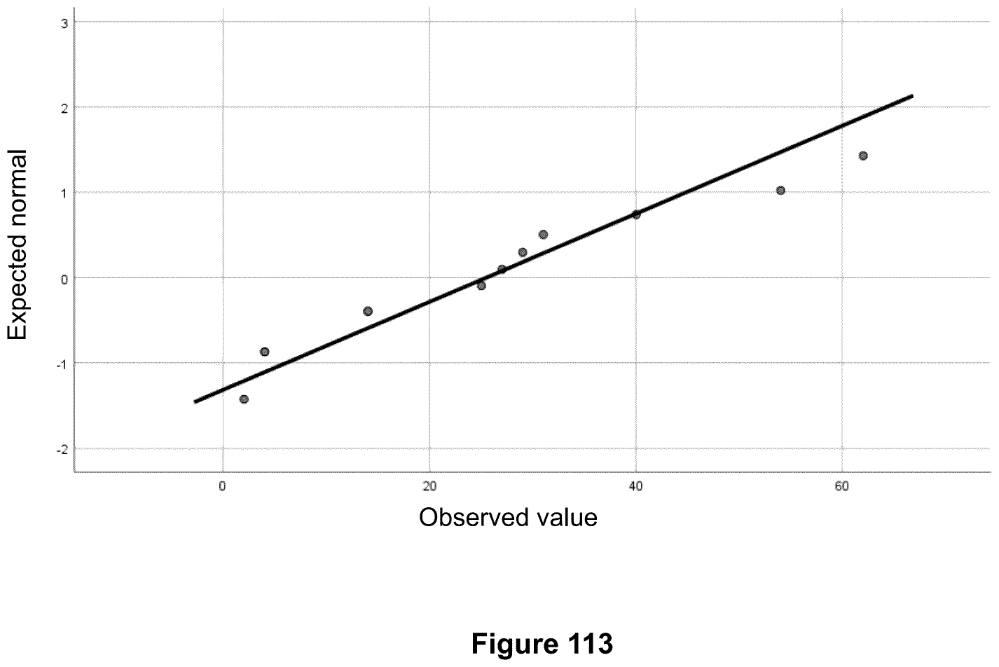

HPLC. The purity of the products was determined by high-performance liquid chromatography (HPLC) on a Shimadzu LC-1OAT high-performance liquid chromatograph equipped with a Perkin Elmer LC-100 oven, containing two Perkin-Elmer PL gel columns of 5×10² and 1×10⁴ Å, a Shimadzu SPD-10A UV detector (X=254 nm), a Shimadzu RID-10A RI-detector, and a PE Nelson Analytical 900 Series integrator data station. All measurements were performed with THF as eluent at a flow rate of 1.0 mL/min at 23° C. (FIG. 109 through FIG. 111).

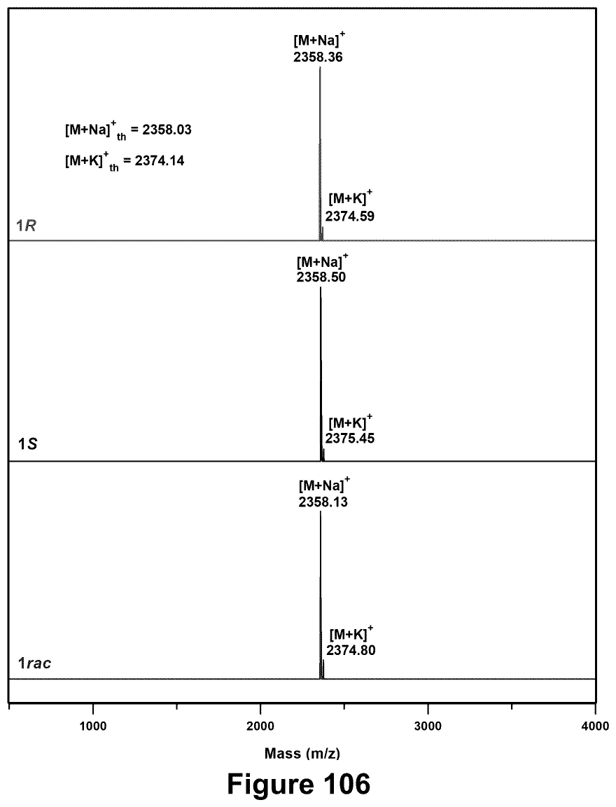

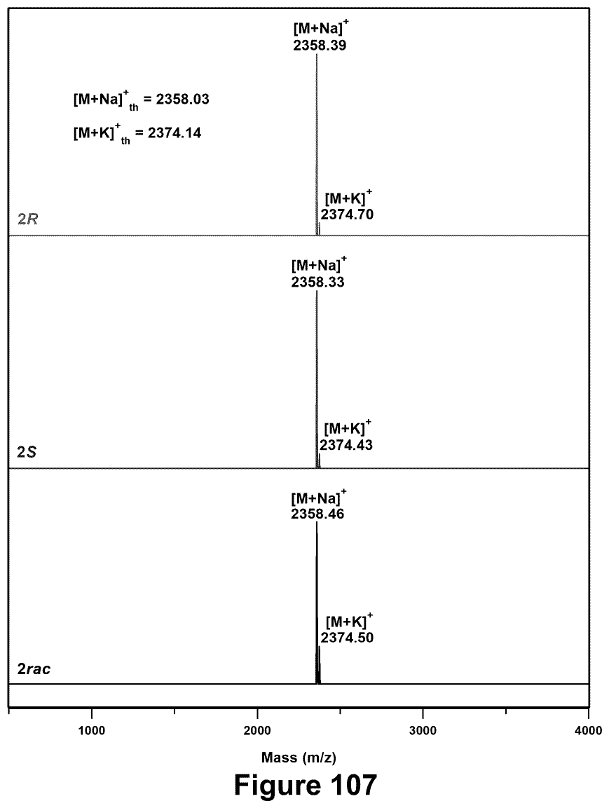

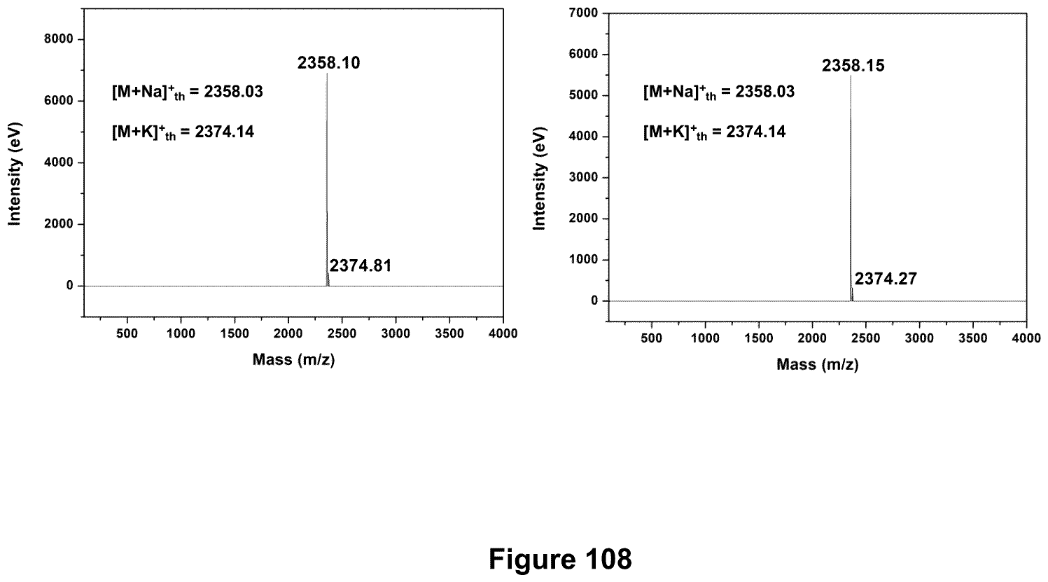

Matrix-assisted laser desorption/ionization time of flight (MALDI-TOF) mass spectrometry. MALDI-TOF mass spectrometry was performed on a PerSeptive Biosystems-Voyager-DE (Framingham, MA) mass spectrometer equipped with a nitrogen laser (337 μm) and operated in linear mode. Internal calibration was performed using Angiotensin II and Bombesin as standards. The analytical samples were prepared by mixing the THF solutions of dendrimer molecules (2-5 mg/mL) and the matrix (2,5-dihydroxybenzoic acid) (10 mg/mL in THF) with a 1:1 to 1:5 v/v ratio. The prepared solution (0.5 μL) was then loaded on a MALDI plate and dried at room temperature before the plate was inserted into the vacuum chamber. The laser intensity and voltage were adjusted depending on the molecular weight and the nature of each analyzed molecule (FIG. 106 through FIG. 108).

Dynamic light scattering (DLS). Hydrodynamic diameters (Dₕ) of the self-assemblies of dendrimer molecules and their size distributions were measured on a Malvern Instruments particle sizer (Zetasizer® Nano S, Malvern Instruments, UK) equipped with 4 mW He—Ne laser (633 nm) and avalanche photodiode positioned at 1750 to the beam and temperature controlled cuvette holder. Experiments were performed at 25° C. in triplicate. The distribution of radius was intensity.

Cryogenic Transmission Electron Microscopy (Cryo-TEM). Morphologies of the Janus Dendrimer assemblies were characterized by cryo-TEM. Images were acquired on a Carl Zeiss Libra 120 microscope. Samples were prepared by plunge freezing of aqueous dispersion on plasma-treated lacey grids. The vitrified specimens were then transferred to a Gatan-910 cryoholder. The 5 images were recorded at −170° C. with an acceleration voltage of 120 kV. The concentration of JD in the aqueous solution of DSs was 1 mg mL-¹.

### Synthesis

### Synthesis of (rac)-2,2-Dimethyl-1,3-dioxolane-4-methanol, 5-Hydroxy-2-phenyl-1,3-dioxane, Isopropylidene-2,2-bis(methoxy)propionic acid, Hydrophilic First-Generation Acid and Hydrophobic First-Generation Acid

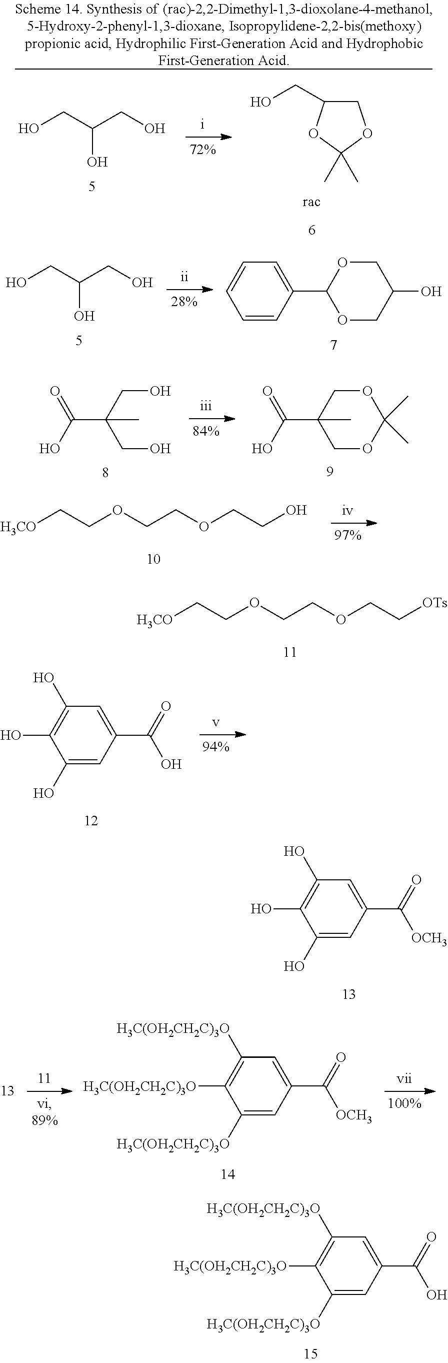

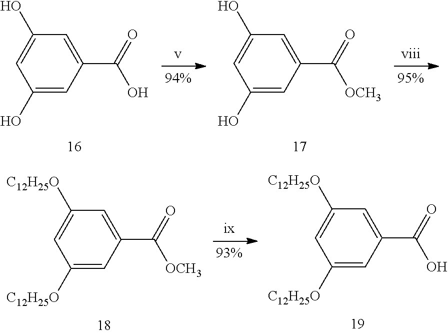

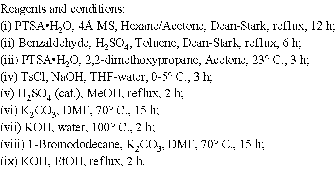

(rac)-2,2-Dimethyl-1,3-dioxolane-4-methanol (6) was synthesized according to a literature procedure (Xu et al., Org. Lett. 2017, 19, 3703-3706). 5-Hydroxy-2-phenyl-1,3-dioxane (7) was synthesized according to a literature procedure (Crich et al., J. Am. Chem. Soc. 1995, 117, 8757-8768). Isopropylidene-2,2-bis(methoxy)propionic acid (9) was synthesized according to a literature procedure (Ihre et al., Macromolecules 1998, 31, 4061-4068; Percec et al., Science 2010, 328, 1009-1014). Hydrophilic first-generation acid (3,4,5)-tris(methyl triethylene glycol)benzoic acid ((3,4,5)-3EO-G1-COOH, 15) and hydrophobic first-generation acid 3,5-bis(dodecyloxy)benzoic acid ((3,5)-12G1-COOH, 19) were prepared according to literature procedures (Percec et al., Science 2010, 328, 1009-1014; Percec et al., J. Am. Chem. Soc. 2013, 135, 4129-4148).

### Synthesis of Hydrophilic and Hydrophobic Second-Generation Acids

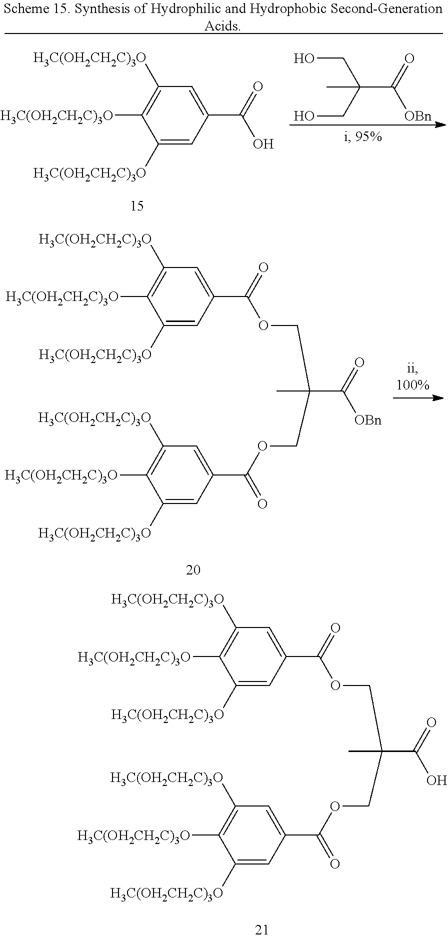

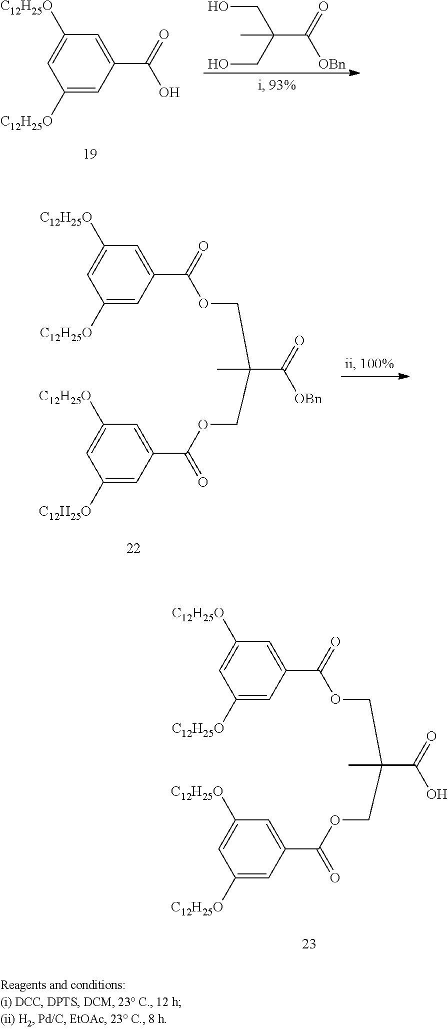

The synthesis of compounds 20, 21, 22 and 23 was adapted from literature procedures (Zhang et al., J. Am. Chem. Soc. 2015, 137, 13334-13344). Benzyl 2,2-bis(3,4,5-tris{2-[2-(2-methoxyethoxy)ethoxy]ethoxy}benzonate)propionate (20). (3,4,5)-3EO-G1-COOH (15, 3.00 g, 4.93 mmol, 2.1 equiv), benzyl 2,2-bis(hydroxymethyl)propionate (0.53 g, 2.35 mmol, 1.0 equiv) and DPTS (1.54 g, 4.93 mmol, 2.1 20 equiv) were dissolved in 10 mL dry DCM. N,N′-Dicyclohexylcarbodiimide (DCC, 1.94 g, 9.40 mmol, 4.0 equiv) dissolved in 2 mL dry DCM was added in one portion to the above mixture. The reaction was allowed to stir at 23° C. for 12 h. The reaction mixture was filtered to remove the urea, which was washed with DCM carefully. The solvent was then removed under vacuum and the crude product was dissolved in 10 mL ethyl acetate (EtOAc). The EtOAc solution was filtered through Celite and the filter cake was washed with EtOAc. The filtrate was concentrated and purified by column chromatography (SiO₂) with EtOAc, then EtOAc/MeOH=20/1, 10/1 as the eluent to give the title compound as a light-yellow oil (3.15 g, 95%). ¹H NMR (500 MHz, CDCl₃) δ 7.24-7.20 (m, 9H, PhH), 5.17 (s, 2H, PhCH₂OCO—), 4.53 (d, 4H, —C((CH₂O—)₂)CH₃), 4.21 (t, 4H, PhOCH₂CH₂O—), 4.13 (t, 8H, PhOCH₂CH₂O—), 3.84 (t, 8H, PhOCH₂CH₂O—), 3.78 (t, 4H, PhOCH₂CH₂O—), 3.73-3.71 (m, 12H, PhOCH₂CH₂OCH₂CH₂—), 3.70-3.61 (m, 24H, PhOCH₂CH₂OCH₂CH₂OCH₂CH₂—), 3.52 (m, 12H, PhOCH₂CH₂OCH₂CH₂OCH₂CH₂—), 3.35 (ds, 18H, —OCH₃), 1.42 (s, 3H, C(CH₂O—)₂CH₃). Purity by HPLC: 99+%. MALDI-TOF MS m/z of [M+Na]⁺ calculated for C₆₈H₁₀₈O₃₀Na: 1428.6; Found: 1428.9.

2,2-Bis(3,4,5-tris{2-[2-(2-methoxyethoxy)ethoxy]ethoxy}benzonate)propionic acid ((3,4,5)-3EO-G1-BMPA-COOH, 21). Compound 20 (1.40 g, 1.00 mmol) was dissolved in 30 mL EtOAc. Then Pd/C (70 mg, 5 wt %) was added and the flask that was evacuated and filled with hydrogen for three times. The mixture was stirred at 23° C. for 8 h under hydrogen atmosphere. The reaction mixture was filtered through Celite and the filter cake was washed with EtOAc. Evaporation of the solvent yielded the title compound as a light-yellow oil (1.31 g, 100%). ¹H NMR (500 MHz, CDCl₃) δ 7.30 (s, 4H, PhH), 4.56 (d, 2H, 0.5×—C((CH₂O—)₂)CH₃), 4.46 (d, 2H, 0.5×—C((CH₂O—)₂)CH₃), 4.21 (m, 12H, PhOCH₂CH₂O—), 3.84 (t, 8H, PhOCH₂CH₂O—), 3.78 (t, 4H, PhOCH₂CH₂O—), 3.75-3.68 (m, 12H, PhOCH₂CH₂OCH₂CH₂—), 3.68-3.60 (m, 24H, PhOCH₂CH₂OCH₂CH₂OCH₂CH₂—), 3.54 (m, 12H, PhOCH₂CH₂OCH₂CH₂OCH₂CH₂—), 3.36 (ds, 18H, —OCH₃), 1.40 (s, 3H, C(CH₂O—)₂CH₃). Purity by HPLC: 99+%. MALDI-TOF MS m/z of [M+Na]⁺ calculated for C₆₁H₁₀₂O₃₀Na: 1338.5; Found: 1338.9.

Benzyl 2,2-bis(3,5-didodecyloxybenzonate)propionate (22). (3,5)-12G1-COOH (19, 1.50 g, 3.06 mmol, 2.1 equiv), benzyl 2,2-bis(hydroxymethyl)propionate (0.33 g, 1.46 mmol, 1.0 equiv) and DPTS (0.96 g, 3.06 mmol, 2.1 equiv) were dissolved in 10 mL dry DCM. DCC (1.20 g, 5.84 mmol, 4.0 equiv) dissolved in 2 mL dry DCM was added in one portion into the above mixture. The reaction was allowed to stir at 23° C. for 12 h. The reaction mixture was filtered to remove the urea, which was washed with DCM carefully. The solvent was then removed under vacuum and the crude product was dissolved in 10 mL EtOAc. The EtOAc solution was filtered through Celite and the filter cake was washed with EtOAc. The filtrate was concentrated and purified by column chromatography (SiO₂) with Hexane/EtOAc=20/1 as the eluent to give the title compound as a light-yellow oil (1.59 g, 93%). ¹H NMR (500 MHz, CDCl₃) δ 7.27-7.23 (m, 5H, PhH), 7.06 (d, 4H, PhH), 6.62 (t, 2H, PhH), 5.18 (s, 2H, PhCH₂OCO—), 4.56 (d, 4H, —C(CH₂O—)₂CH₃), 3.92 (t, 8H, PhOCH₂(CH₂)₁₀CH₃), 1.77 (m, 8H, PhOCH₂CH₂(CH₂)₉CH₃), 1.44 (m, 8H, PhOCH₂CH₂CH₂(CH₂)₈CH₃), 1.44 (s, 3H, —C(CH₂O—)₂CH₃), 1.26 (br, 64H, PhOCH₂CH₂CH₂(CH₂)₈CH₃), 0.87 (t, 12H, PhO(CH₂)₁₁CH₃). Purity by HPLC: 99+%. MALDI-TOF MS m/z of [M+Na]⁺ calculated for C₇₄H₁₂₀O₁₀Na: 1192.8; Found: 1193.3. 2,2-Bis(3,5-didodecyloxybenzonate)propionic acid ((3,5)-12G1-BMPA-COOH, 23). Compound 22 (1.50 g, 1.30 mmol) was dissolved in 30 mL EtOAc. Then Pd/C (75 mg, 5 wt %) was added and the flask was evacuated and filled three times with hydrogen. The mixture was stirred at 23° C. for 8 h under hydrogen atmosphere. The reaction mixture was filtered through Celite and the filter cake was washed with EtOAc. Evaporation of the solvent yielded the title compound as a colorless oil (1.40 g, 100%). ¹H NMR (500 MHz, CDCl₃) δ 7.09 (d, 4H, PhH), 6.61 (t, 2H, PhH), 4.56 (m, 4H, —C((CH₂O—)₂)CH₃), 3.93 (t, 8H, PhOCH₂(CH₂)₁₀CH₃), 1.76 (m, 8H, PhOCH₂CH₂(CH₂)₉CH₃), 1.45 (s, 3H, —C(CH₂O—)₂CH₃), 1.43 (m, 8H, PhOCH₂CH₂CH₂(CH₂)₈CH₃), 1.26 (br, 64H, PhOCH₂CH₂CH₂(CH₂)₈CH₃), 0.88 (t, 12H, PhO(CH₂)₁₁CH₃). Purity by HPLC: 99+%. MALDI-TOF MS m/z of [M+Na]⁺ calculated for C₆₇H₁₁₄O₁₀Na: 1102.6; Found: 1102.9.

### Synthesis of Hydrophilic Homochiral/Racemic Minidendron

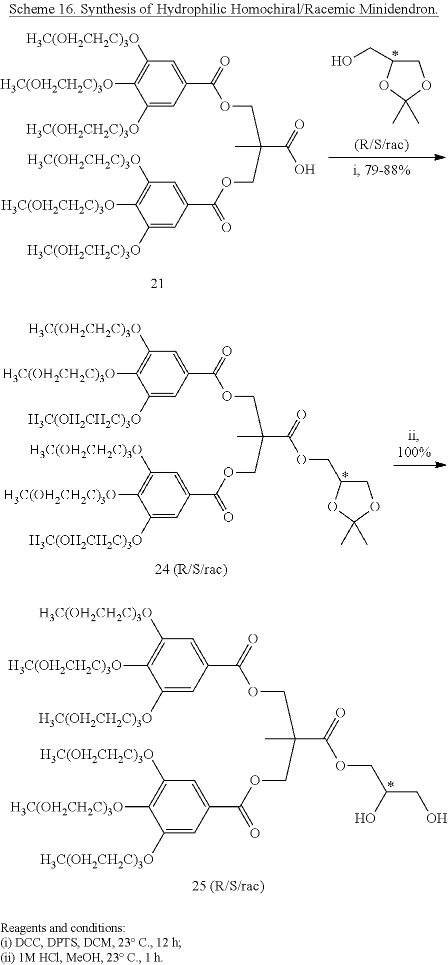

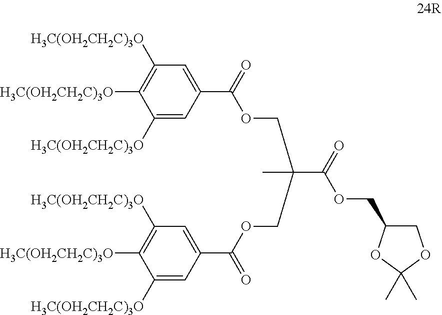

(3,4,5)-3EO-G1-BMPA-GC-(R)-(1,2)acetonide (24R). (3,4,5)-3EO-G1-BMPA-COOH (21, 0.40 g, 0.30 mmol, 1.0 equiv), (S)-2,2-dimethyl-1,3-dioxolane-4-methanol (0.044 g, 0.33 mmol, 1.1 equiv) and DPTS (0.103 g, 0.33 mmol, 1.1 equiv) were dissolved in 5 mL dry DCM. DCC (0.093 g, 0.45 mmol, 1.5 equiv) dissolved in 2 mL dry DCM was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with EtOAc, then DCM/MeOH=20/1 as the eluent to give the title compound as a colorless oil (0.38 g, 88%). ¹H NMR (500 MHz, CDCl₃) δ 7.23 (s, 4H, PhH), 4.53 (m, 4H, —C((CH₂O—)₂)CH₃), 4.26 (m, 1H, CH(O—)(CH₂O—)₂), 4.23 (q, 1H, 0.5×(—COO—CH₂O—CH(O—)—)), 4.21 (t, 4H, PhOCH₂CH₂O—), 4.19 (t, 8H, PhOCH₂CH₂O—), 4.15 (q, 1H, 0.5×(—COO—CH₂O—CH(O—)—)), 3.99 (q, 1H, 0.5×(—CH(O—)—CH₂O—)), 3.86 (t, 8H, PhOCH₂CH₂O—), 3.78 (t, 4H, PhOCH₂CH₂O—), 3.75-3.70 (m, 12H, PhOCH₂CH₂OCH₂CH₂—), 3.70 (q, 1H, 0.5×(—CH(O—)—CH₂O—)), 3.67-3.61 (m, 24H, PhOCH₂CH₂OCH₂CH₂OCH₂CH₂—), 3.53 (m, 12H, PhOCH₂CH₂OCH₂CH₂OCH₂CH₂—), 3.36 (ds, 18H, —OCH₃), 1.42 (s, 3H, —C(CH₂O—)₂CH₃), 1.36 (s, 3H, C(O-)₂(CH₃)CH₃), 1.26 (s, 3H, C(O-)₂(CH₃)CH₃). ¹³C NMR (500 MHz, CDCl₃) δ 172.2, 165.1, 152.2, 142.8, 124.1, 109.5, 108.9, 73.0, 72.2, 71.7, 70.6, 70.5, 70.3, 70.3, 69.4, 68.7, 65.9, 65.6, 65.0, 58.8, 46.8, 26.5, 24.9, 17.8.

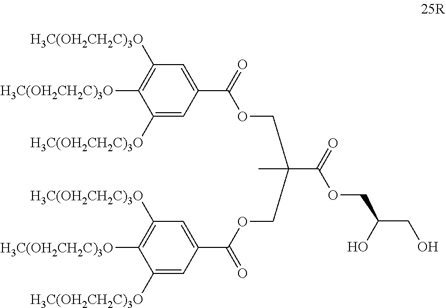

(3,4,5)-3EO-G1-BMPA-GC-(R)-(1,2)(OH)₂ (25R). (3,4,5)-3EO-G1-BMPA-GC-(R)-(1,2)acetonide (24R, 0.22 g, 0.15 mmol) was dissolved in 10 mL MeOH. Then HCl (1 M, 2.0 mL, 2.0 mmol) was added. The mixture was stirred at 23° C. for 1 h. The reaction mixture was extracted by DCM (3×20 mL) and the organic phase was dried over anhydrous magnesium sulfate. Filtration and evaporation of the solvent yielded the title compound as a colorless oil (0.21 g, 100%). ¹H NMR (500 MHz, CDCl₃) δ 7.27 (d, 4H, PhH), 4.58 (q, 2H, —C((CH₂O—)₂)CH₃), 4.48 (q, 2H, —C((CH₂O—)₂)CH₃), 4.29 (q, 1H, 0.5×(—COO—CH₂O—CH(O—)—)), 4.20 (m, 13H, 0.5×(—COO—CH₂O—CH(O—)-) and PhOCH₂CH₂O—), 3.91 (m, 1H, CH(O—)(CH₂O-)₂), 3.86 (t, 8H, PhOCH₂CH₂O—), 3.78 (t, 4H, PhOCH₂CH₂O—), 3.75-3.69 (m, 12H, PhOCH₂CH₂OCH₂CH₂—), 3.70 (q, 1H, 0.5×(—CH(O—)—CH₂O—)), 3.68-3.60 (m, 25H, PhOCH₂CH₂OCH₂CH₂OCH₂CH₂— and 0.5×(—CH(O—)—CH₂O—)), 3.53 (m, 12H, PhOCH₂CH₂OCH₂CH₂OCH₂CH₂—), 3.36 (ds, 18H, —OCH₃), 1.41 (s, 3H, —C(CH₂O—)₂CH₃). ¹³C NMR (500 MHz, CDCl₃) δ 172.9, 165.4, 152.3, 143.0, 124.2, 109.3, 72.4, 71.8, 71.8, 70.7, 70.7, 70.6, 70.6, 70.5, 70.4, 69.9, 69.5, 68.8, 66.1, 63.3, 58.9, 47.1, 18.0.

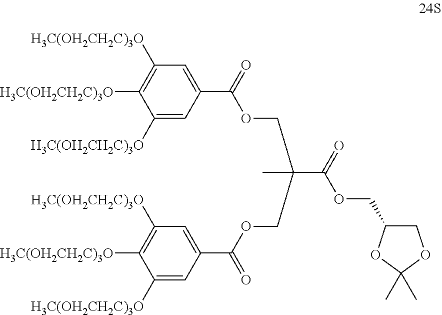

(3,4,5)-3EO-G1-BMPA-GC-(S)-(1,2)acetonide (24S). Compound 24S was synthesized from (3,4,5)-3EO-G1-BMPA-COOH (21, 0.40 g, 0.30 mmol, 1.0 equiv) and (R)-2,2-dimethyl-1,3-dioxolane-4-methanol (0.044 g, 0.33 mmol, 1.1 equiv) following a procedure similar to that used for the synthesis of 24R. The title compound was obtained as a colorless oil (0.37 g, 86%). ¹H NMR (500 MHz, CDCl₃) δ 7.23 (s, 4H, PhH), 4.53 (m, 4H, —C((CH₂O—)₂)CH₃), 4.26 (m, 1H, CH(O—)(CH₂O—)₂), 4.24 (q, 1H, 0.5×(—COO—CH₂O—CH(O—)—)), 4.21 (t, 4H, PhOCH₂CH₂O—), 4.17 (t, 8H, PhOCH₂CH₂O—), 4.15 (q, 1H, 0.5×(—COO—CH₂O—CH(O—)—)), 3.99 (q, 1H, 0.5×(—CH(O—)—CH₂O—)), 3.86 (t, 8H, PhOCH₂CH₂O—), 3.78 (t, 4H, PhOCH₂CH₂O—), 3.74-3.70 (m, 12H, PhOCH₂CH₂OCH₂CH₂—), 3.70 (q, 1H, 0.5×(—CH(O—)—CH₂O—)), 3.67-3.61 (m, 24H, PhOCH₂CH₂OCH₂CH₂OCH₂CH₂—), 3.53 (m, 12H, PhOCH₂CH₂OCH₂CH₂OCH₂CH₂—), 3.36 (ds, 18H, —OCH₃), 1.42 (s, 3H, —C(CH₂O—)₂CH₃), 1.36 (s, 3H, C(O-)₂(CH₃)CH₃), 1.27 (s, 3H, C(O-)₂(CH₃)CH₃). ¹³C NMR (500 MHz, CDCl₃) δ 172.3, 165.2, 152.2, 142.8, 124.1, 109.5, 108.9, 73.0, 72.2, 71.7, 70.6, 70.5, 70.3, 70.3, 69.4, 68.7, 65.9, 65.6, 65.0, 58.8, 46.8, 26.5, 24.9, 17.8.

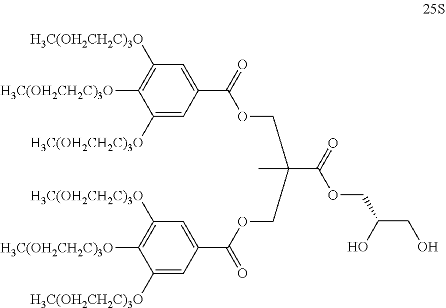

(3,4,5)-3EO-G1-BMPA-GC-(S)-(1,2)(OH)₂ (25S). Compound 25S was synthesized from (3,4,5)-3EO-G1-BMPA-GC-(S)-(1,2)acetonide (24S, 0.36 g, 0.25 mmol) following a procedure similar to that used for the synthesis of 25R. The title compound was obtained as a colorless oil (0.35 g, 100%). ¹H NMR (500 MHz, CDCl₃) δ 7.26 (d, 4H, PhH), 4.56 (q, 2H, —C((CH₂O—)₂)CH₃), 4.48 (q, 2H, —C((CH₂O—)₂)CH₃), 4.27 (q, 1H, 0.5×(—COO—CH₂O—CH(O—)—)), 4.20 (m, 13H, 0.5×(—COO—CH₂O—CH(O—)-) and PhOCH₂CH₂O—), 3.90 (m, 1H, CH(O—)(CH₂O—)₂), 3.85 (t, 8H, PhOCH₂CH₂O—), 3.78 (t, 4H, PhOCH₂CH₂O—), 3.75-3.69 (m, 12H, PhOCH₂CH₂OCH₂CH₂—), 3.69 (q, 1H, 0.5×(—CH(O—)—CH₂O—)), 3.67-3.60 (m, 25H, PhOCH₂CH₂OCH₂CH₂OCH₂CH₂— and 0.5×(—CH(O—)—CH₂O—)), 3.53 (m, 12H, PhOCH₂CH₂OCH₂CH₂OCH₂CH₂—), 3.36 (ds, 18H, —OCH₃), 1.40 (s, 3H, —C(CH₂O—)₂CH₃). ¹³C NMR (500 MHz, CDCl₃) δ 172.8, 165.3, 152.2, 142.9, 142.9, 124.2, 109.2, 109.1, 72.3, 71.7, 71.7, 70.6, 70.6, 70.5, 70.5, 70.4, 70.3, 70.3, 69.7, 69.5, 68.7, 66.0, 65.9, 63.2, 58.8, 47.0, 17.9.

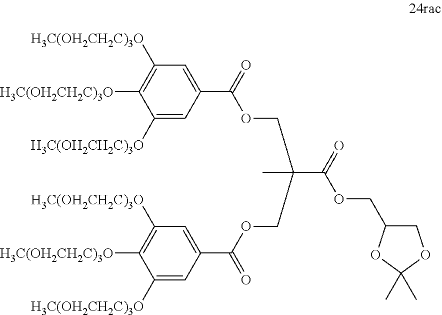

(3,4,5)-3EO-G1-BMPA-GC-(rac)-(1,2)acetonide (24rac). Compound 24rac was synthesized from (3,4,5)-3EO-G1-BMPA-COOH (21, 0.45 g, 0.34 mmol, 1.0 equiv) and (rac)-2,2-dimethyl-1,3-dioxolane-4-methanol (0.049 g, 0.37 mmol, 1.1 equiv) following a procedure similar to that used for the synthesis of 24R. The title compound was obtained as a colorless oil (0.38 g, 79%). ¹H NMR (500 MHz, CDCl₃) δ 7.23 (s, 4H, PhH), 4.53 (m, 4H, —C((CH₂O—)₂)CH₃), 4.26 (m, 1H, CH(O—)(CH₂O—)₂), 4.23 (q, 1H, 0.5×(—COO—CH₂O—CH(O—)—)), 4.21 (t, 4H, PhOCH₂CH₂O—), 4.17 (t, 8H, PhOCH₂CH₂O—), 4.15 (q, 1H, 00.5×(—COO—CH₂O—CH(O—)—)), 3.99 (q, 1H, 0.5×(—CH(O—)—CH₂O—)), 3.86 (t, 8H, PhOCH₂CH₂O—), 3.78 (t, 4H, PhOCH₂CH₂O—), 3.74-3.70 (m, 12H, PhOCH₂CH₂OCH₂CH₂—), 3.70 (q, 1H, 0.5×(—CH(O—)—CH₂O—)), 3.67-3.61 (m, 24H, PhOCH₂CH₂OCH₂CH₂OCH₂CH₂—), 3.53 (m, 12H, PhOCH₂CH₂OCH₂CH₂OCH₂CH₂—), 3.36 (ds, 18H, —OCH₃), 1.42 (s, 3H, —C(CH₂O—)₂CH₃), 1.36 (s, 3H, C(O-)₂(CH₃)CH₃), 1.26 (s, 3H, C(O-)₂(CH₃)CH₃). ¹³C NMR (500 MHz, CDCl₃) δ 172.3, 165.2, 152.3, 142.9, 124.2, 109.6, 109.0, 73.1, 72.3, 71.8, 70.7, 70.5, 70.4, 69.5, 68.8, 66.0, 65.7, 65.1, 58.9, 46.8, 26.6, 25.0, 17.9.

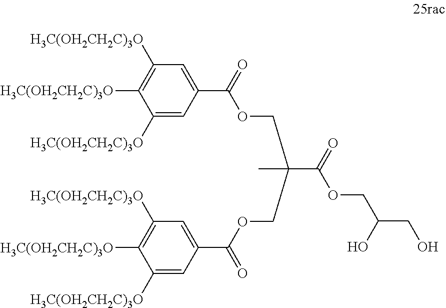

(3,4,5)-3EO-G1-BMPA-GC-(rac)-(1,2)(OH)₂ (25rac). Compound 25rac was synthesized from (3,4,5)-3EO-G1-BMPA-GC-(rac)-(1,2)acetonide (24rac, 0.31 g, 0.22 mmol) following a procedure similar to that used for the synthesis of 25R. The title compound was obtained as a colorless oil (0.30 g, 100%). ¹H NMR (500 MHz, CDCl₃) δ 7.27 (d, 4H, PhH), 4.57 (q, 2H, —C((CH₂O—)₂)CH₃), 4.48 (q, 2H, —C((CH₂O—)₂)CH₃), 4.27 (q, 1H, 0.5×(—COO—CH₂O—CH(O—)—)), 4.20 (m, 13H, 0.5×(—COO—CH₂O—CH(O—)—)) and PhOCH₂CH₂O—), 3.91 (m, 1H, CH(O—)(CH₂O—)₂), 3.86 (t, 8H, PhOCH₂CH₂O—), 3.78 (t, 4H, PhOCH₂CH₂O—), 3.75-3.69 (m, 12H, PhOCH₂CH₂OCH₂CH₂—), 3.70 (q, 1H, 0.5×(—CH(O—)—CH₂O—)), 3.68-3.60 (m, 25H, PhOCH₂CH₂OCH₂CH₂OCH₂CH₂— and 0.5×(—CH(O—)—CH₂O—)), 3.53 (m, 12H, PhOCH₂CH₂OCH₂CH₂OCH₂CH₂—), 3.36 (ds, 18H, —OCH₃), 1.41 (s, 3H, —C(CH₂O—)₂CH₃). ¹³C NMR (500 MHz, CDCl₃) δ 172.9, 165.4, 152.3, 142.9, 124.2, 109.3, 109.2, 72.3, 71.8, 71.8, 70.7, 70.7, 70.6, 70.5, 70.5, 70.4, 70.4, 70.4, 69.8, 69.5, 68.8, 66.1, 66.0, 63.2, 58.9, 47.0, 17.9.

### Synthesis of Hydrophobic Homochiral/Racemic Minidendron

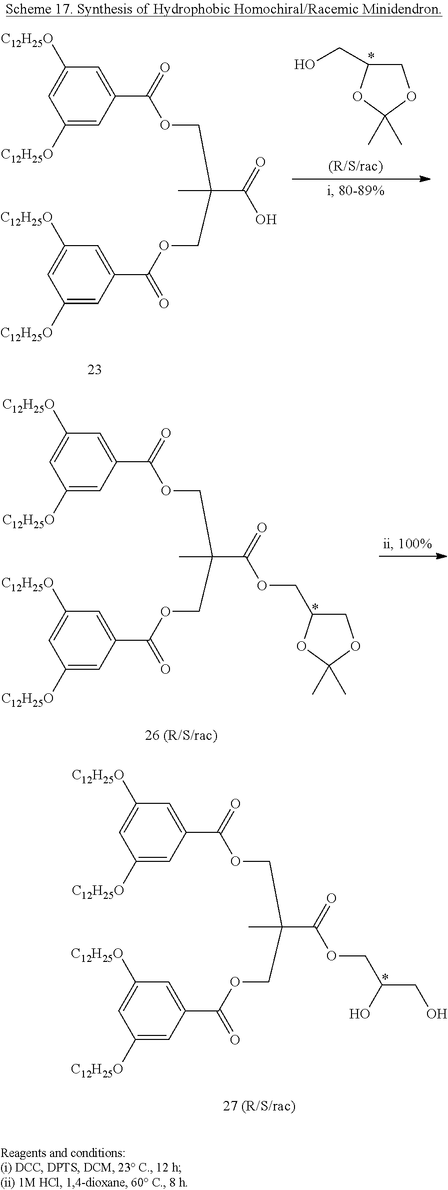

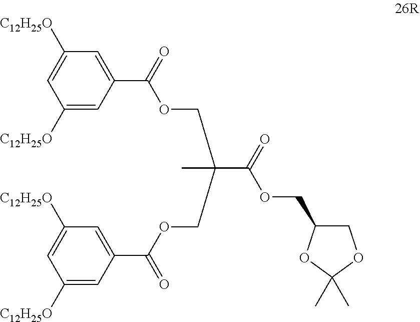

(3,5)-12G1-BMPA-GC-(R)-(1,2)acetonide (26R). (3,5)-12G1-BMPA-COOH (23, 0.40 g, 0.37 mmol, 1.0 equiv), (S)-2,2-dimethyl-1,3-dioxolane-4-methanol (0.054 g, 0.41 mmol, 1.1 equiv) and DPTS (0.13 g, 0.41 mmol, 1.1 equiv) were dissolved in 5 mL dry DCM. DCC (0.12 g, 0.56 mmol, 1.5 equiv) dissolved in 2 mL of dry DCM was added in one portion to the above mixture. The reaction was allowed to stir at 23° C. for 12 h. The reaction mixture was filtered to remove the urea, which was washed with DCM carefully. The filtrate was concentrated and purified by column chromatography (SiO₂) with Hexane/EtOAc=20/1 as the eluent to give the title compound as a colorless oil (0.38 g, 86%). ¹H NMR (500 MHz, CDCl₃) δ 7.10 (d, 4H, PhH), 6.62 (t, 2H, PhH), 4.56 (m, 4H, —C((CH₂O—)₂)CH₃), 4.27 (m, 1H, CH(O—)(CH₂O—)₂), 4.23 (q, 1H, 0.5×(—COO—CH₂O—CH(O—)—)), 4.16 (q, 1H, 0.5×(—COO—CH₂O—CH(O—)—)), 3.99 (q, 1H, 0.5×(—CH(O—)—CH₂O—)), 3.94 (t, 8H, PhOCH₂(CH₂)₁₀CH₃), 3.71 (q, 1H, 0.5×(—CH(O—)—CH₂O—)), 1.77 (m, 8H, PhOCH₂CH₂(CH₂)₉CH₃), 1.44 (m, 8H, PhOCH₂CH₂CH₂(CH₂)₈CH₃), 1.43 (s, 3H, —C(CH₂O—)₂CH₃), 1.38 (s, 3H, C(O-)₂(CH₃)CH₃), 1.27 (s, 3H, C(O-)₂(CH₃)CH₃), 1.26 (br, 64H, PhOCH₂CH₂CH₂(CH₂)₈CH₃), 0.88 (t, 12H, PhO(CH₂)₁₁CH₃). ¹³C NMR (500 MHz, CDCl₃) δ 172.6, 165.9, 160.3, 131.3, 131.3, 109.8, 107.8, 106.6, 73.3, 68.4, 66.4, 66.1, 66.1, 65.4, 47.0, 32.0, 29.8, 29.7, 29.7, 29.7, 29.5, 29.5, 29.3, 26.8, 26.1, 25.2, 22.8, 18.1, 14.2.

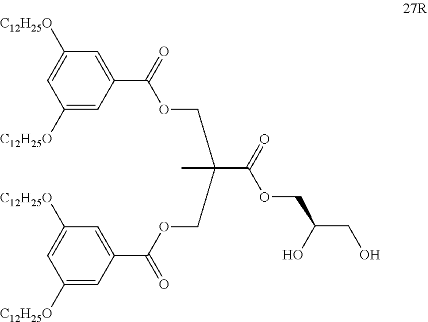

(3,5)-12G1-BMPA-GC-(R)-(1,2)(OH)₂ (27R). (3,5)-12G1-BMPA-GC-(R)-(1,2)acetonide (26R, 0.29 g, 0.24 mmol) was dissolved in 10 mL 1,4-dioxane. Then HCl (2 M, 2.0 mL, 4.0 mmol) was added. The mixture was stirred at 60° C. for 8 h. The reaction mixture was extracted by DCM (3×20 mL) and the organic phase was dried over anhydrous magnesium sulfate. Filtration and evaporation of the solvent yielded the title compound as a colorless oil (0.28 g, 100%). ¹H NMR (500 MHz, CDCl₃) δ 7.11 (d, 4H, PhH), 6.63 (t, 2H, PhH), 4.58 (m, 4H, —C((CH₂O—)₂)CH₃), 4.27 (q, 1H, 0.5×(—COO—CH₂O—CH(O—)—)), 4.21 (q, 1H, 0.5×(—COO—CH₂O—CH(O—)—)), 3.94 (t, 8H, PhOCH₂(CH₂)₁₀CH₃), 3.92 (m, 1H, CH(O—)(CH₂O—)₂), 3.65 (q, 1H, 0.5×(—CH(O—)—CH₂O—)), 3.60 (q, 1H, 0.5×(—CH(O—)—CH₂O—)), 2.74 (s, 1H, —CH(OH)(CH₂O-)₂), 2.17 (s, 1H, —CH₂OH), 1.77 (m, 8H, PhOCH₂CH₂(CH₂)₉CH₃), 1.44 (m, 8H, PhOCH₂CH₂CH₂(CH₂)₈CH₃), 1.44 (s, 3H, —C(CH₂O—)₂CH₃), 1.26 (br, 64H, PhOCH₂CH₂CH₂(CH₂)₈CH₃), 0.88 (t, 12H, PhO(CH₂)₁₁CH₃). ¹³C NMR (500 MHz, CDCl₃) δ 173.0, 166.0, 166.0, 160.2, 131.2, 131.2, 107.8, 106.6, 69.9, 68.4, 66.1, 63.2, 47.1, 32.0, 29.7, 29.7, 29.7, 29.7, 29.5, 29.4, 29.2, 26.1, 22.8, 18.1, 14.2.

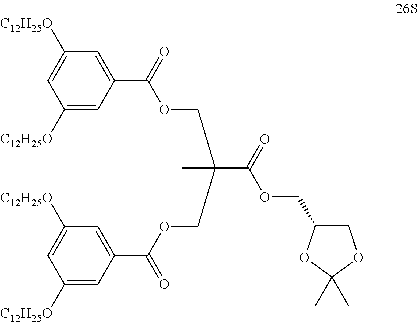

(3,5)-12G1-BMPA-GC-(S)-(1,2)acetonide (26S). Compound 26S was synthesized from (3,5)-12G1-BMPA-COOH (23, 0.40 g, 0.37 mmol, 1.0 equiv) and (R)-2,2-dimethyl-1,3-dioxolane-4-methanol (0.054 g, 0.41 mmol, 1.1 equiv) following a procedure similar to that used for the synthesis of 26R. The title compound was obtained as a colorless oil (0.39 g, 89%). ¹H NMR (500 MHz, CDCl₃) δ 7.10 (d, 4H, PhH), 6.62 (t, 2H, PhH), 4.56 (m, 4H, —C((CH₂O—)₂)CH₃), 4.27 (m, 1H, CH(O—)(CH₂O—)₂), 4.23 (q, 1H, 0.5×(—COO—CH₂O—CH(O—)—)), 4.16 (q, 1H, 0.5×(—COO—CH₂O—CH(O—)—)), 3.99 (q, 1H, 0.5×(—CH(O—)—CH₂O—)), 3.94 (t, 8H, PhOCH₂(CH₂)₁₀CH₃), 3.72 (q, 1H, 0.5×(—CH(O—)—CH₂O—)), 1.77 (m, 8H, PhOCH₂CH₂(CH₂)₉CH₃), 1.44 (m, 8H, PhOCH₂CH₂CH₂(CH₂)₈CH₃), 1.43 (s, 3H, —C(CH₂O-)₂CH₃), 1.38 (s, 3H, C(O—)₂(CH₃)CH₃), 1.27 (s, 3H, C(O—)₂(CH₃)CH₃), 1.26 (br, 64H, PhOCH₂CH₂CH₂(CH₂)₈CH₃), 0.88 (t, 12H, PhO(CH₂)₁₁CH₃). ¹³C NMR (500 MHz, CDCl₃) δ 172.6, 165.9, 160.3, 131.4, 131.3, 109.8, 107.8, 106.6, 73.3, 68.4, 66.4, 66.2, 66.1, 65.4, 47.0, 32.0, 29.8, 29.8, 29.7, 29.7, 29.5, 29.5, 29.3, 26.8, 26.1, 25.2, 22.8, 18.1, 14.2.

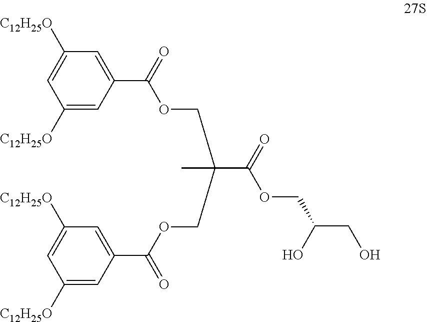

(3,5)-12G1-BMPA-GC-(S)-(1,2)(OH)₂ (27S). Compound 27S was synthesized from (3,5)-12G1-BMPA-GC-(S)-(1,2)acetonide (26S, 0.31 g, 0.26 mmol) following a procedure similar to that used for the synthesis of 27R. The title compound was obtained as a colorless oil (0.30 g, 100%). ¹H NMR (500 MHz, CDCl₃) δ 7.11 (d, 4H, PhH), 6.64 (t, 2H, PhH), 4.58 (m, 4H, —C((CH₂O—)₂)CH₃), 4.27 (q, 1H, 0.5×(—COO—CH₂O—CH(O—)—)), 4.21 (q, 1H, 0.5×(—COO—CH₂O—CH(O—)—)), 3.94 (t, 8H, PhOCH₂(CH₂)₁₀CH₃), 3.91 (m, 1H, CH(O—)(CH₂O—)₂), 3.65 (q, 1H, 0.5×(—CH(O—)—CH₂O—)), 3.59 (q, 1H, 0.5×(—CH(O—)—CH₂O—)), 2.73 (s, 1H, —CH(OH)(CH₂O—)₂), 2.16 (s, 1H, —CH₂OH), 1.77 (m, 8H, PhOCH₂CH₂(CH₂)₉CH₃), 1.44 (m, 8H, PhOCH₂CH₂CH₂(CH₂)₈CH₃), 1.44 (s, 3H, —C(CH₂O—)₂CH₃), 1.26 (br, 64H, PhOCH₂CH₂CH₂(CH₂)₈CH₃), 0.88 (t, 12H, PhO(CH₂)₁₁CH₃). ¹³C NMR (500 MHz, CDCl₃) δ 173.0, 166.1, 166.1, 160.3, 131.2, 131.2, 107.8, 106.6, 70.0, 68.4, 66.2, 63.2, 47.2, 32.0, 29.8, 29.7, 29.7, 29.7, 29.5, 29.5, 29.3, 26.1, 22.8, 18.1, 14.2.

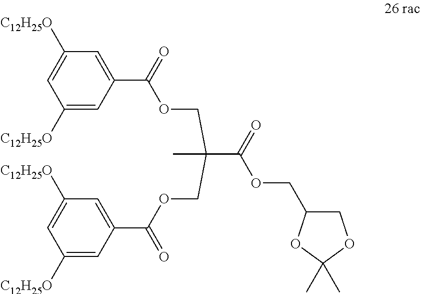

(3,5)-12G1-BMPA-GC-(rac)-(1,2)acetonide (26rac). Compound 26rac was synthesized from (3,5)-12G1-BMPA-COOH (23, 0.53 g, 0.49 mmol, 1.0 equiv) and (rac)-2,2-dimethyl-1,3-dioxolane-4-methanol (0.071 g, 0.54 mmol, 1.1 equiv) following a procedure similar to that used for the synthesis of 26R. The title compound was obtained as a colorless oil (0.46 g, 80%). ¹H NMR (500 MHz, CDCl₃) δ 7.10 (d, 4H, PhH), 6.62 (t, 2H, PhH), 4.56 (m, 4H, —C((CH₂O—)₂)CH₃), 4.27 (m, 1H, CH(O—)(CH₂O—)₂), 4.23 (q, 1H, 0.5×(—COO—CH₂O—CH(O—)—)), 4.16 (q, 1H, 0.5×(—COO—CH₂O—CH(O—)—)), 3.99 (q, 1H, 0.5×(—CH(O—)—CH₂O—)), 3.94 (t, 8H, PhOCH₂(CH₂)₁₀CH₃), 3.71 (q, 1H, 0.5×(—CH(O—)—CH₂O—)), 1.77 (m, 8H, PhOCH₂CH₂(CH₂)₉CH₃), 1.44 (m, 8H, PhOCH₂CH₂CH₂(CH₂)₈CH₃), 1.43 (s, 3H, —C(CH₂O—)₂CH₃), 1.38 (s, 3H, C(O—)₂(CH₃)CH₃), 1.27 (s, 3H, C(O—)₂(CH₃)CH₃), 1.26 (br, 64H, PhOCH₂CH₂CH₂(CH₂)₈CH₃), 0.88 (t, 12H, PhO(CH₂)₁₁CH₃). ¹³C NMR (500 MHz, CDCl₃) δ 172.6, 165.9, 160.3, 131.4, 131.3, 109.8, 107.8, 106.6, 73.3, 68.4, 66.4, 66.2, 66.1, 65.4, 47.0, 32.0, 29.8, 29.8, 29.7, 29.7, 29.5, 29.5, 29.3, 26.8, 26.1, 25.2, 22.8, 18.1, 14.2.

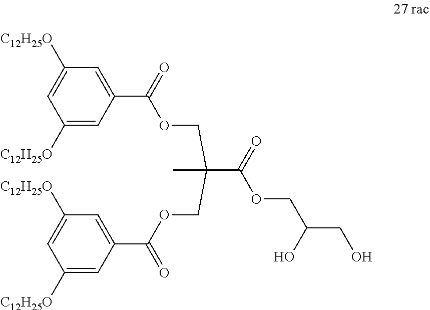

(3,5)-12G1-BMPA-GC-(rac)-(1,2)(OH)₂ (27rac). Compound 27rac was synthesized from (3,5)-12G1-BMPA-GC-(rac)-(1,2)acetonide (26rac, 0.30 g, 0.25 mmol) following a procedure similar to that used for the synthesis of 27R. The title compound was obtained as a colorless oil (0.29 g, 100%). ¹H NMR (500 MHz, CDCl₃) δ 7.11 (d, 4H, PhH), 6.64 (t, 2H, PhH), 4.58 (m, 4H, —C((CH₂O—)₂)CH₃), 4.27 (q, 1H, 0.5×(—COO—CH₂O—CH(O—)—)), 4.21 (q, 1H, 0.5×(—COO—CH₂O—CH(O—)—)), 3.95 (t, 8H, PhOCH₂(CH₂)₁₀CH₃), 3.91 (m, 1H, CH(O—)(CH₂O—)₂), 3.65 (q, 1H, 0.5×(—CH(O—)—CH₂O—)), 3.59 (q, 1H, 0.5×(—CH(O—)—CH₂O—)), 2.72 (s, 1H, —CH(OH)(CH₂O—)₂), 2.15 (s, 1H, —CH₂OH), 1.77 (m, 8H, PhOCH₂CH₂(CH₂)₉CH₃), 1.44 (m, 8H, PhOCH₂CH₂CH₂(CH₂)₈CH₃), 1.44 (s, 3H, —C(CH₂O—)₂CH₃), 1.26 (br, 64H, PhOCH₂CH₂CH₂(CH₂)₈CH₃), 0.88 (t, 12H, PhO(CH₂)₁₁CH₃). ¹³C NMR (500 MHz, CDCl₃) δ 173.0, 166.1, 166.1, 160.3, 131.2, 131.2, 107.9, 106.6, 70.0, 68.4, 66.2, 63.2, 47.2, 32.0, 29.8, 29.7, 29.7, 29.7, 29.5, 29.5, 29.3, 26.1, 22.8, 18.1, 14.2.

### Synthesis of Amphiphilic Glycerol-Based Janus Dendrimers from Library 1

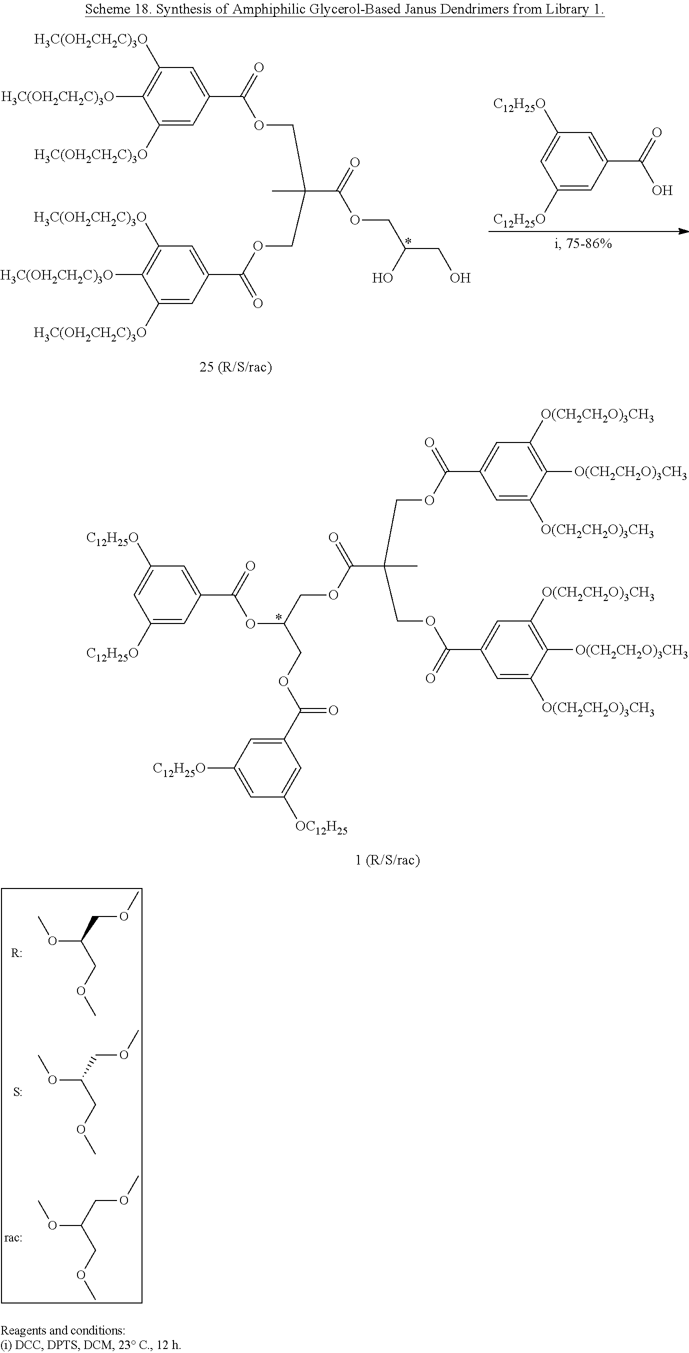

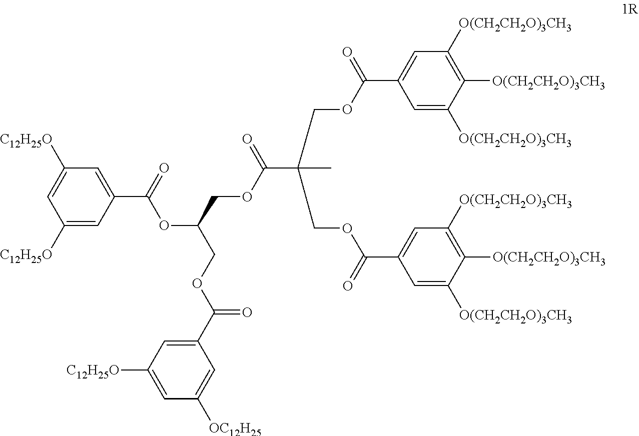

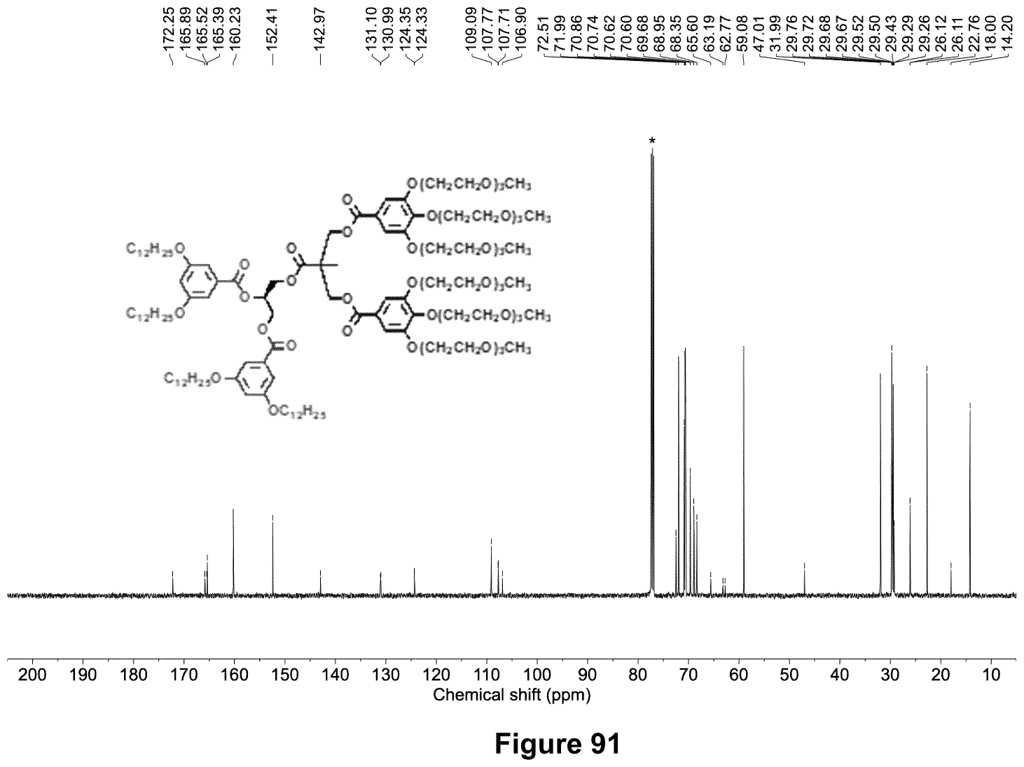

(3,5)-12G1-GC-(R)-BMPA-(3,4,5)-3EO-G1 (1R). (3,4,5)-3EO-G1-BMPA-GC-(R)-(1,2)(OH)₂ (25R, 0.18 g, 0.13 mmol, 1.0 equiv), (3,5)-12G1-COOH (0.15 g, 0.30 mmol, 2.3 equiv) and DPTS (0.094 g, 0.30 mmol, 2.3 equiv) were dissolved in 5 mL dry DCM. DCC (0.080 g, 0.39 mmol, 3.0 equiv) dissolved in 2 mL dry DCM was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with EtOAc/MeOH=20/1 as the eluent to give the title compound as a light-yellow oil (0.23 g, 86%). ¹H NMR (500 MHz, CDCl₃) δ 7.20 (s, 4H, PhH), 7.07 (t, 4H, PhH), 6.60 (t, 1H, PhH), 6.58 (t, 1H, PhH), 5.66 (m, 1H, CH(O—)(CH₂O—)₂), 4.63 (q, 1H, 0.25×—C((CH₂O—)₂)CH₃), 4.58-4.42 (m, 7H, 0.75×—C((CH₂O-)₂)CH₃, —COO—CH₂O—CH(O—)- and —CH(O—)—CH₂O—), 4.20 (t, 4H, PhOCH₂CH₂O—), 4.15 (t, 8H, PhOCH₂CH₂O—), 3.89 (m, 8H, PhOCH₂CH₂O—), 3.84 (t, 8H, PhOCH₂(CH₂)₁₀CH₃), 3.78 (t, 4H, PhOCH₂CH₂O—), 3.74-3.69 (m, 12H, PhOCH₂CH₂OCH₂CH₂—), 3.67-3.60 (m, 24H, PhOCH₂CH₂OCH₂CH₂OCH₂CH₂—), 3.53 (m, 12H, PhOCH₂CH₂OCH₂CH₂OCH₂CH₂—), 3.36 (ds, 18H, —OCH₃), 1.73 (m, 8H, PhOCH₂CH₂(CH₂)₉CH₃), 1.41 (m, 8H, PhOCH₂CH₂CH₂(CH₂)₈CH₃), 1.38 (s, 3H, —C(CH₂O—)₂CH₃), 1.25 (br, 64H, PhOCH₂CH₂CH₂(CH₂)₈CH₃), 0.87 (t, 12H, PhO(CH₂)₁₁CH₃). ¹³C NMR (500 MHz, CDCl₃) δ 172.3, 165.9, 165.5, 165.4, 160.2, 152.4, 143.0, 131.1, 131.0, 124.4, 124.3, 109.1, 107.8, 107.7, 106.9, 72.5, 72.0, 70.9, 70.7, 70.6, 70.6, 69.7, 69.0, 68.4, 65.6, 63.2, 62.8, 59.1, 47.0, 32.0, 29.8, 29.7, 29.7, 29.7, 29.5, 29.5, 29.4, 29.3, 29.3, 26.1, 26.1, 22.8, 18.0, 14.2. Purity by HPLC: 99+%. MALDI-TOF MS m/z of [M+Na]⁺ calculated for C₁₂₆H₂₁₂O₃₈Na: 2358.0; Found: 2358.3. The ¹H NMR and ¹³C NMR spectra of compound 1R are shown in FIG. 90 and FIG. 91, respectively.

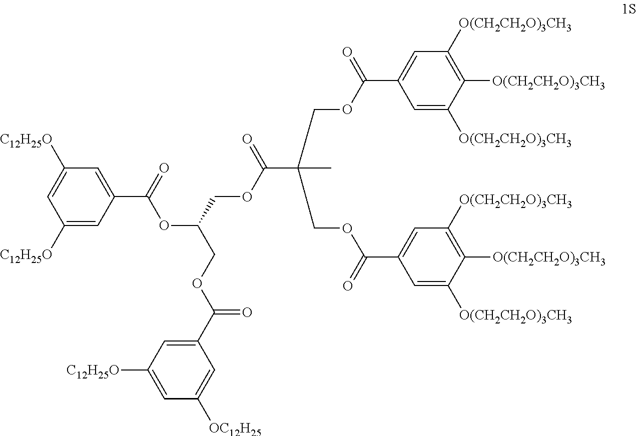

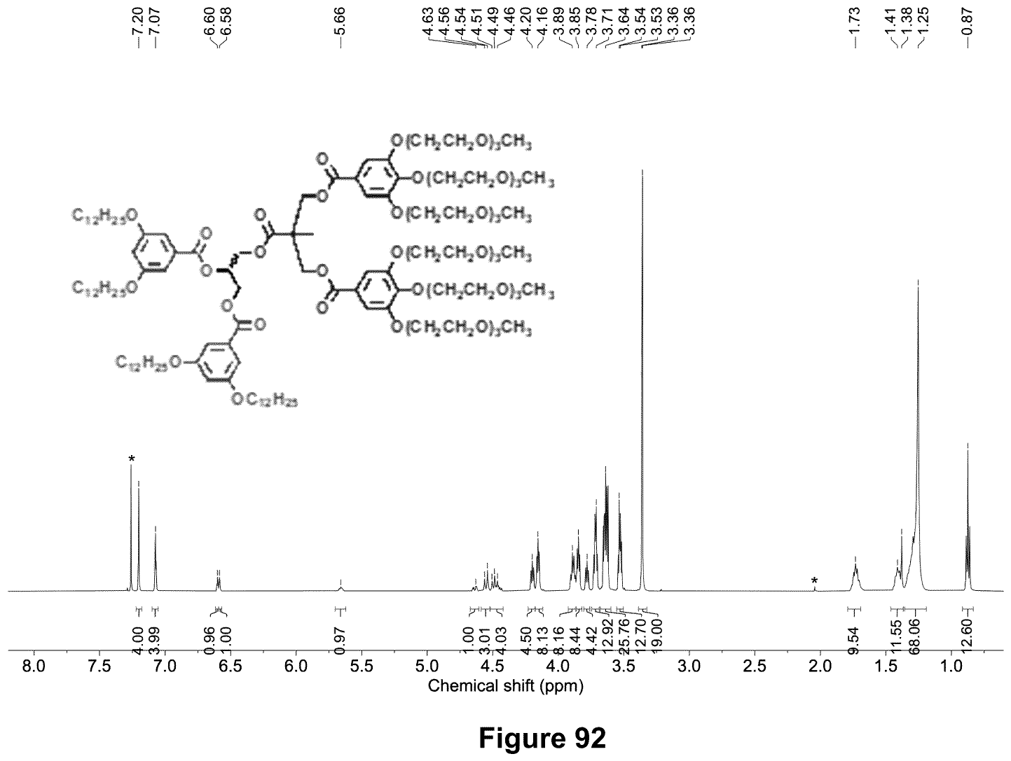

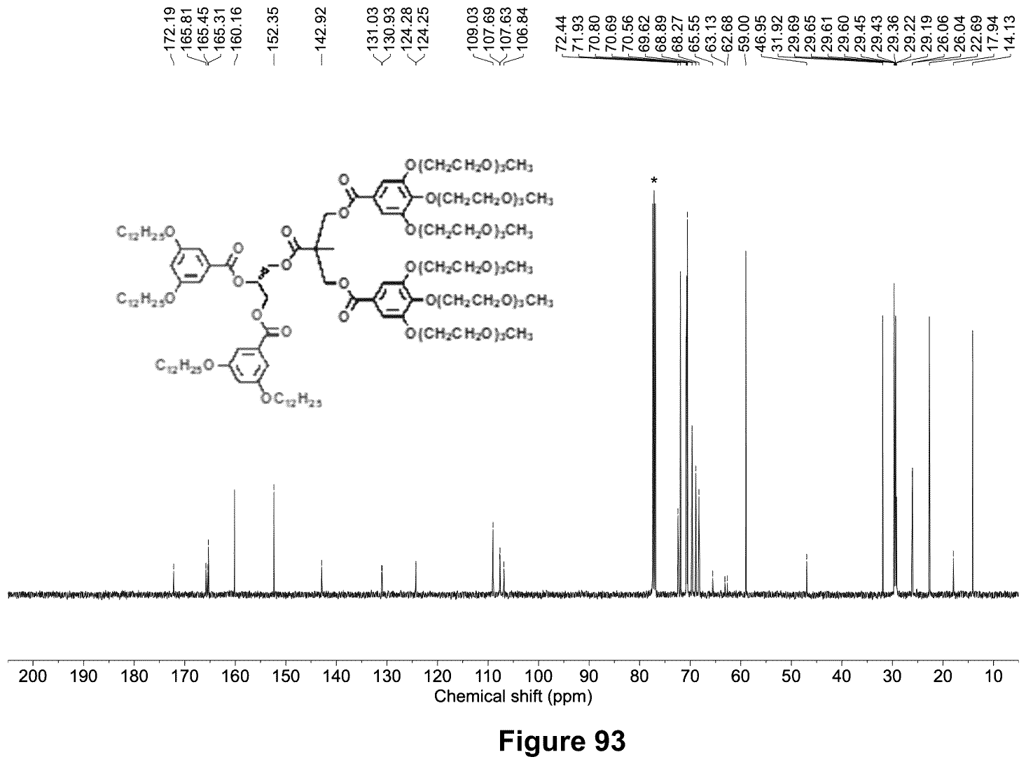

(3,5)-12G1-GC-(S)-BMPA-(3,4,5)-3EO-G1 (1S). Compound 1S was synthesized from (3,4,5)-3EO-G1-BMPA-GC-(S)-(1,2)(OH)₂ (25S, 0.20 g, 0.14 mmol, 1.0 equiv) and (3,5)-12G1-COOH (0.16 g, 0.32 mmol, 2.3 equiv) following a procedure similar to that used for the synthesis of 1R. The title compound was obtained as a light-yellow oil (0.26 g, 80%). ¹H NMR (500 MHz, CDCl₃) δ 7.20 (s, 4H, PhH), 7.07 (t, 4H, PhH), 6.60 (t, 1H, PhH), 6.58 (t, 1H, PhH), 5.66 (m, 1H, CH(O—)(CH₂O—)₂), 4.63 (q, 1H, 0.25×—C((CH₂O-)₂)CH₃), 4.58-4.42 (m, 7H, 0.75×—C((CH₂O—)₂)CH₃, —COO—CH₂O—CH(O—)- and —CH(O—)—CH₂O—), 4.20 (t, 4H, PhOCH₂CH₂O—), 4.16 (t, 8H, PhOCH₂CH₂O—), 3.89 (m, 8H, PhOCH₂CH₂O—), 3.85 (t, 8H, PhOCH₂(CH₂)₁₀CH₃), 3.78 (t, 4H, PhOCH₂CH₂O—), 3.74-3.69 (m, 12H, PhOCH₂CH₂OCH₂CH₂—), 3.67-3.60 (m, 24H, PhOCH₂CH₂OCH₂CH₂OCH₂CH₂—), 3.53 (m, 12H, PhOCH₂CH₂OCH₂CH₂OCH₂CH₂—), 3.36 (ds, 18H, —OCH₃), 1.73 (m, 8H, PhOCH₂CH₂(CH₂)₉CH₃), 1.41 (m, 8H, PhOCH₂CH₂CH₂(CH₂)₈CH₃), 1.38 (s, 3H, —C(CH₂O-)₂CH₃), 1.25 (br, 64H, PhOCH₂CH₂CH₂(CH₂)₈CH₃), 0.87 (t, 12H, PhO(CH₂)₁₁CH₃). ¹³C NMR (500 MHz, CDCl₃) δ 172.2, 165.8, 165.5, 165.3, 160.2, 152.4, 142.9, 131.0, 130.9, 124.3, 124.3, 109.0, 107.8, 107.6, 106.8, 72.4, 71.9, 70.8, 70.7, 70.6, 69.6, 68.9, 68.3, 65.6, 63.1, 62.7, 59.0, 47.0, 31.9, 29.7, 29.7, 29.6, 29.6, 29.5, 29.4, 29.4, 29.2, 29.2, 26.1, 26.0, 22.7, 17.9, 14.1. Purity by HPLC: 99+%. MALDI-TOF MS m/z of [M+Na]⁺ calculated for C₁₂₆H₂₁₂O₃₈Na: 2358.0; Found: 2358.4. The ¹H NMR and ¹³C NMR spectra of compound 1S are shown in FIG. 92 and FIG. 93, respectively.

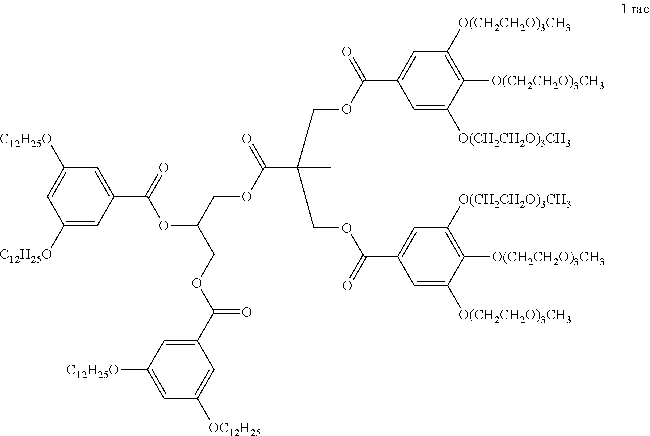

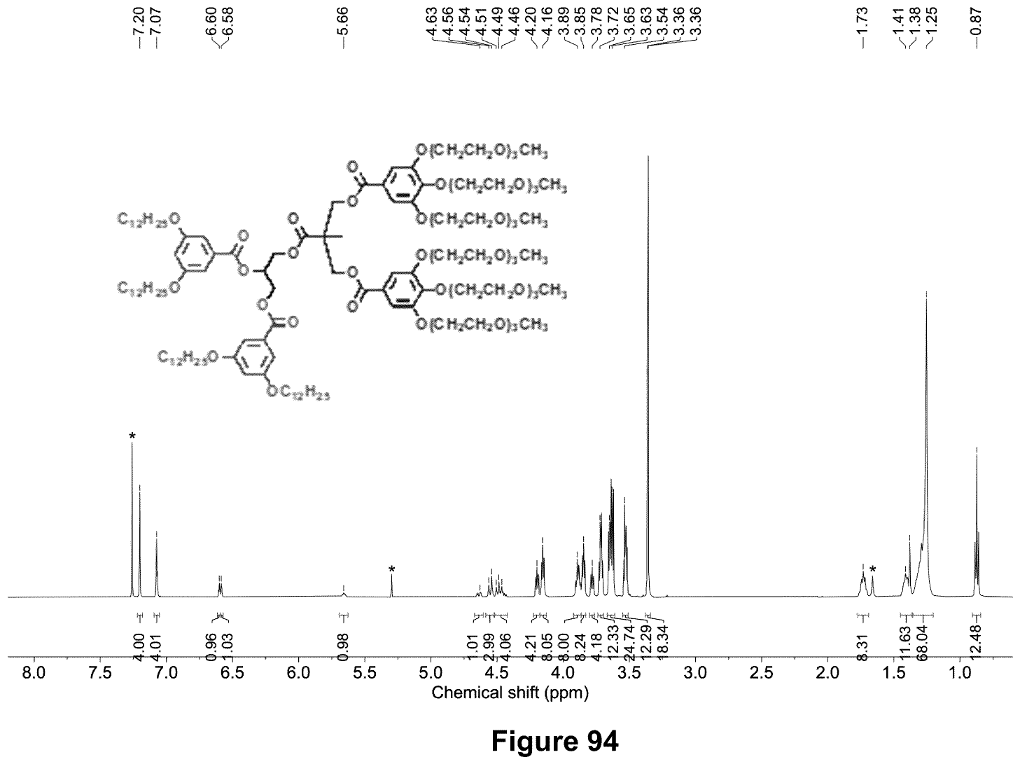

(3,5)-12G1-GC-(rac)-BMPA-(3,4,5)-3EO-G1 (1rac). Compound 1rac was synthesized from (3,4,5)-3EO-G1-BMPA-GC-(rac)-(1,2)(OH)₂ (25rac, 0.28 g, 0.20 mmol, 1.0 equiv) and (3,5)-12G1-COOH (0.23 g, 0.46 mmol, 2.3 equiv) following a procedure similar to that used for the synthesis of 1R. The title compound was obtained as a light-yellow oil (0.35 g, 75%). ¹H NMR (500 MHz, CDCl₃) δ 7.20 (s, 4H, PhH), 7.07 (t, 4H, PhH), 6.60 (t, 1H, PhH), 6.58 (t, 1H, PhH), 5.66 (m, 1H, CH(O—)(CH₂O—)₂), 4.63 (q, 1H, 0.25×—C((CH₂O-)₂)CH₃), 4.58-4.42 (m, 7H, 0.75×—C((CH₂O—)₂)CH₃, —COO—CH₂O—CH(O—)- and —CH(O—)—CH₂O—), 4.20 (t, 4H, PhOCH₂CH₂O—), 4.16 (t, 8H, PhOCH₂CH₂O—), 3.89 (m, 8H, PhOCH₂CH₂O—), 3.85 (t, 8H, PhOCH₂(CH₂)₁₀CH₃), 3.78 (t, 4H, PhOCH₂CH₂O—), 3.74-3.69 (m, 12H, PhOCH₂CH₂OCH₂CH₂—), 3.67-3.60 (m, 24H, PhOCH₂CH₂OCH₂CH₂OCH₂CH₂—), 3.53 (m, 12H, PhOCH₂CH₂OCH₂CH₂OCH₂CH₂—), 3.36 (ds, 18H, —OCH₃), 1.73 (m, 8H, PhOCH₂CH₂(CH₂)₉CH₃), 1.41 (m, 8H, PhOCH₂CH₂CH₂(CH₂)₈CH₃), 1.38 (s, 3H, —C(CH₂O-)₂CH₃), 1.25 (br, 64H, PhOCH₂CH₂CH₂(CH₂)₈CH₃), 0.87 (t, 12H, PhO(CH₂)₁₁CH₃). ¹³C NMR (500 MHz, CDCl₃) δ 172.2, 165.9, 165.5, 165.4, 160.2, 152.4, 143.0, 131.1, 131.0, 124.3, 124.3, 109.1, 107.8, 107.7, 106.9, 72.5, 72.0, 70.9, 70.7, 70.6, 70.6, 69.7, 68.9, 68.3, 65.6, 63.2, 62.8, 59.1, 47.0, 32.0, 29.8, 29.7, 29.7, 29.7, 29.5, 29.5, 29.4, 29.3, 29.3, 26.1, 26.1, 22.8, 18.0, 14.2. Purity by HPLC: 99+%. MALDI-TOF MS m/z of [M+Na]⁺ calculated for C₁₂₆H₂₁₂O₃₈Na: 2358.0; Found: 2358.1. The ¹H NMR and ¹³C NMR spectra of compound 1rac are shown in FIG. 94 and FIG. 95, respectively.

### Synthesis of Amphiphilic Glycerol-Based Janus Dendrimer from Library 2

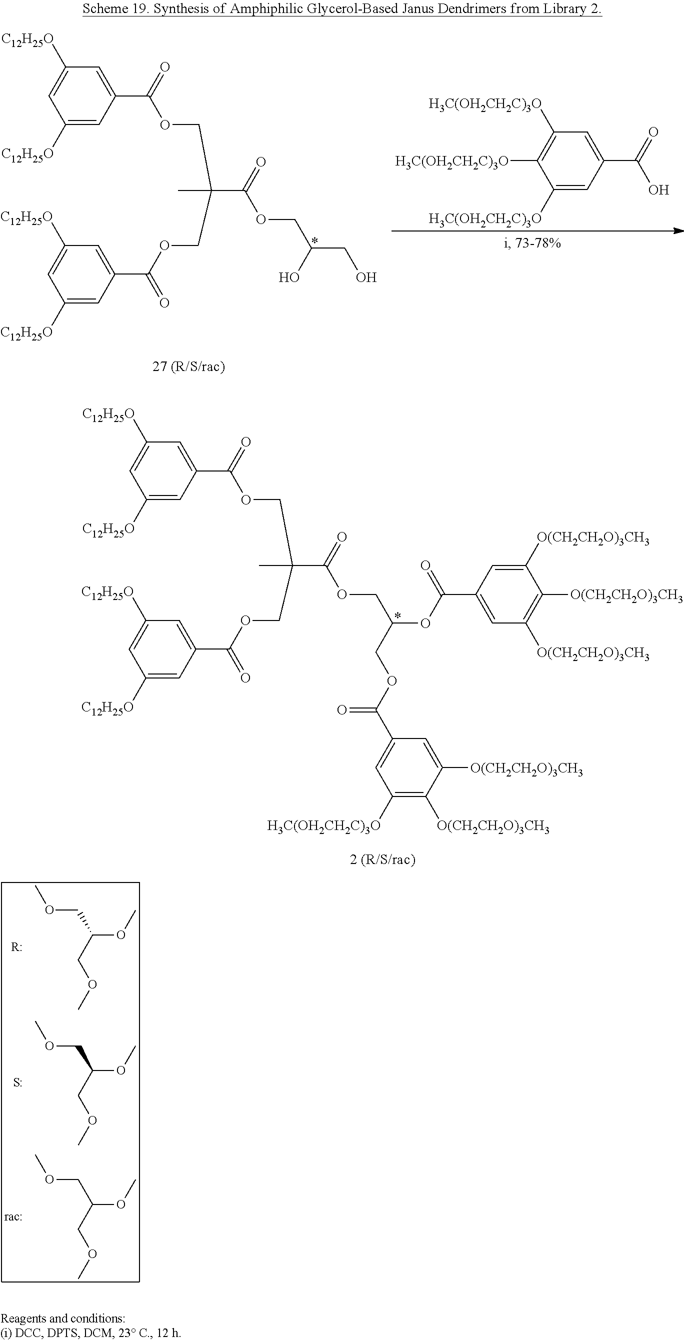

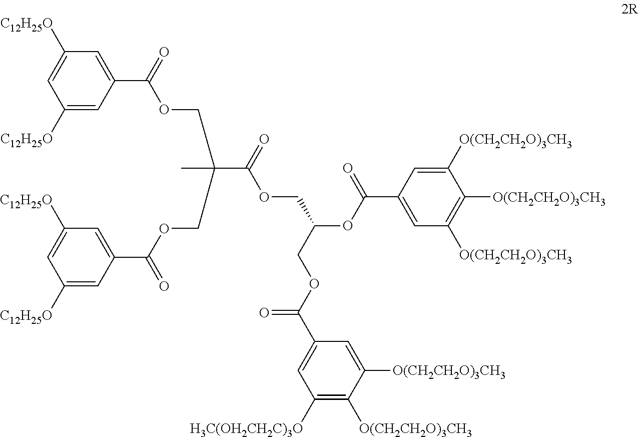

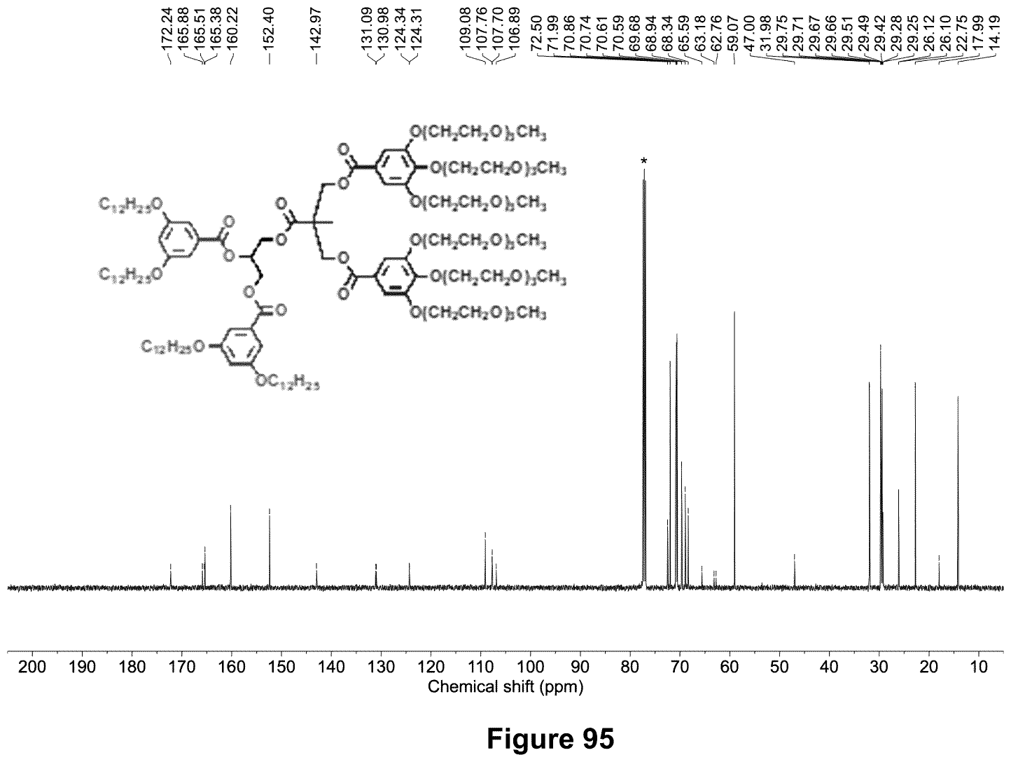

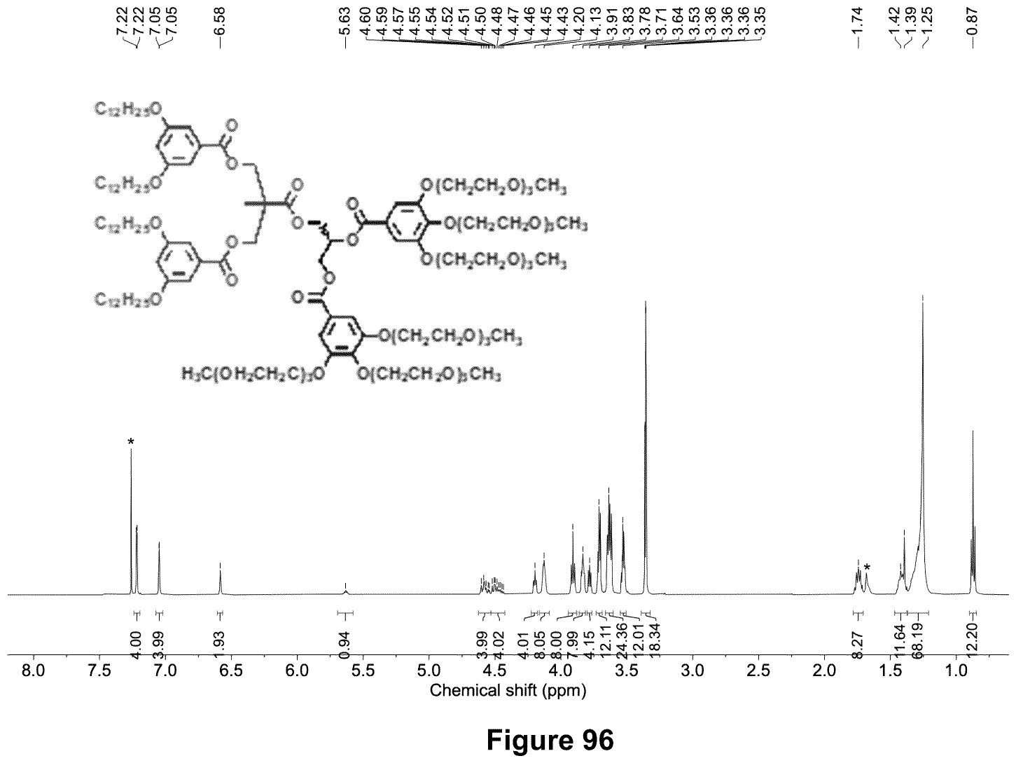

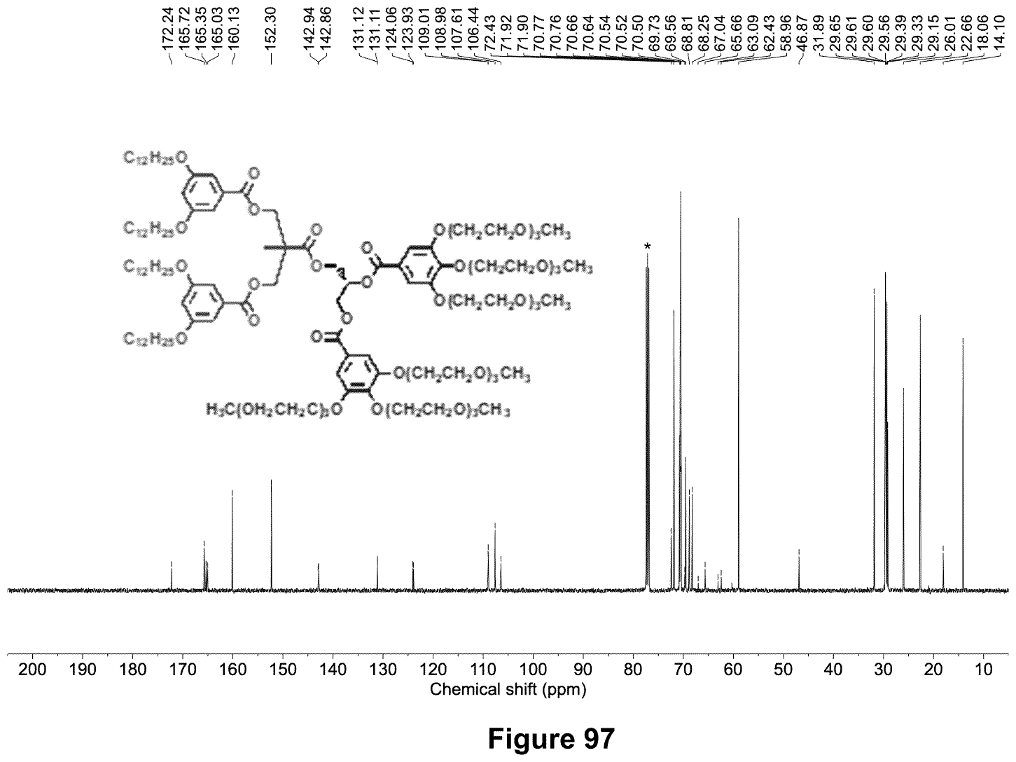

(3,5)-12G1-BMPA-GC-(R)-(3,4,5)-3EO-G1 (2R). (3,5)-12G1-BMPA-GC-(R)-(1,2)(OH)₂ (27R, 0.28 g, 0.24 mmol, 1.0 equiv), (3,4,5)-3EO-G1-COOH (0.33 g, 0.55 mmol, 2.3 equiv) and DPTS (0.17 g, 0.55 mmol, 2.3 equiv) were dissolved in 6 mL dry DCM. DCC (0.15 g, 0.72 mmol, 3.0 equiv) dissolved in 2 mL dry DCM was added in one portion into the above mixture. The reaction was allowed to stir at 23° C. for 12 h. The reaction mixture was filtered to remove the urea, which was washed with DCM carefully. The filtrate was concentrated and purified by column chromatography (SiO₂) with EtOAc/MeOH=20/1 as the eluent to give the title compound as a light-yellow oil (0.41 g, 73%). ¹H NMR (500 MHz, CDCl₃) δ 7.22 (d, 4H, PhH), 7.05 (d, 2H, PhH), 7.05 (d, 2H, PhH), 6.58 (t, 2H, PhH), 5.63 (m, 1H, CH(O—)(CH₂O—)₂), 4.62-4.42 (m, 8H, —C((CH₂O—)₂)CH₃, —COO—CH₂O—CH(O—)- and —CH(O—)—CH₂O—), 4.20 (m, 4H, PhOCH₂CH₂O—), 4.13 (m, 8H, PhOCH₂CH₂O—), 3.91 (t, 8H, PhOCH₂CH₂O—), 3.83 (m, 8H, PhOCH₂(CH₂)₁₀CH₃), 3.78 (t, 4H, PhOCH₂CH₂O—), 3.74-3.68 (m, 12H, PhOCH₂CH₂OCH₂CH₂—), 3.67-3.60 (m, 24H, PhOCH₂CH₂OCH₂CH₂OCH₂CH₂—), 3.53 (m, 12H, PhOCH₂CH₂OCH₂CH₂OCH₂CH₂—), 3.38-3.33 (m, 18H, —OCH₃), 1.74 (m, 8H, PhOCH₂CH₂(CH₂)₉CH₃), 1.42 (m, 8H, PhOCH₂CH₂CH₂(CH₂)₈CH₃), 1.39 (s, 3H, —C(CH₂O-)₂CH₃), 1.25 (br, 64H, PhOCH₂CH₂CH₂(CH₂)₈CH₃), 0.87 (t, 12H, PhO(CH₂)₁₁CH₃). ¹³C NMR (500 MHz, CDCl₃) δ 172.2, 165.7, 165.4, 165.0, 160.1, 152.3, 142.9, 142.9, 131.1, 131.1, 124.1, 123.9, 109.0, 109.0, 107.6, 106.4, 72.4, 71.9, 71.9, 70.8, 70.8, 70.7, 70.6, 70.5, 70.5, 70.5, 69.7, 69.6, 68.8, 68.3, 67.0, 65.7, 63.1, 62.4, 59.0, 46.9, 31.9, 29.7, 29.6, 29.6, 29.6, 29.4, 29.3, 29.2, 26.0, 22.7, 18.1, 14.1. Purity by HPLC: 99+%. MALDI-TOF MS m/z of [M+Na]⁺ calculated for C₁₂₆H₂₁₂O₃₈Na: 2358.0; Found: 2358.4. The ¹H NMR and ¹³C NMR spectra of compound 2R are shown in FIG. 96 and FIG. 97, respectively.

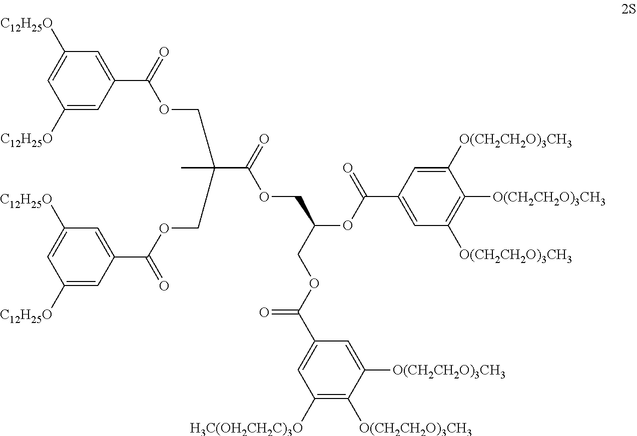

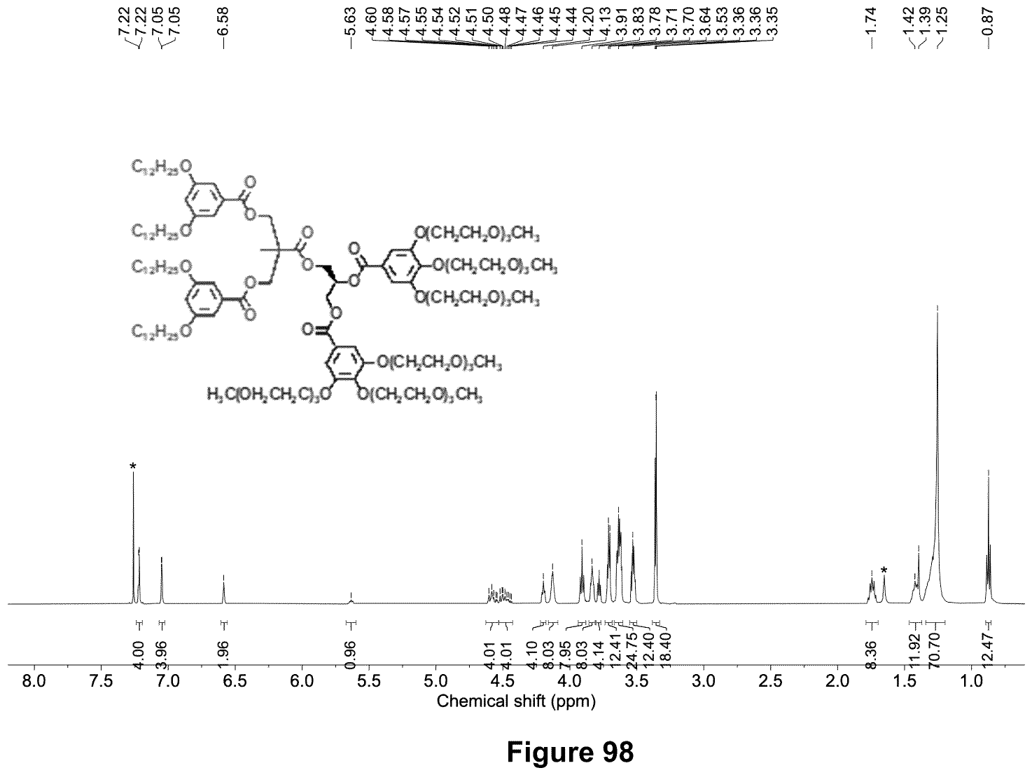

(3,5)-12G1-BMPA-GC-(S)-(3,4,5)-3EO-G1 (2S). Compound 2S was synthesized from (3,5)-12G1-BMPA-GC-(S)-(1,2)(OH)₂ (27S, 0.24 g, 0.21 mmol, 1.0 equiv) and (3,4,5)-3EO-G1-COOH (0.29 g, 0.48 mmol, 2.3 equiv) following a procedure similar to that used for the synthesis of 2R. The title compound was obtained as a light-yellow oil (0.37 g, 76%). ¹H NMR (500 MHz, CDCl₃) δ 7.22 (d, 4H, PhH), 7.05 (d, 2H, PhH), 7.05 (d, 2H, PhH), 6.58 (t, 2H, PhH), 5.63 (m, 1H, CH(O—)(CH₂O—)₂), 4.63-4.42 (m, 8H, —C((CH₂O—)₂)CH₃, —COO—CH₂O—CH(O—)- and —CH(O—)—CH₂O—), 4.20 (m, 4H, PhOCH₂CH₂O—), 4.13 (m, 8H, PhOCH₂CH₂O—), 3.91 (t, 8H, PhOCH₂CH₂O—), 3.83 (m, 8H, PhOCH₂(CH₂)₁₀CH₃), 3.78 (t, 4H, PhOCH₂CH₂O—), 3.74-3.68 (m, 12H, PhOCH₂CH₂OCH₂CH₂—), 3.67-3.60 (m, 24H, PhOCH₂CH₂OCH₂CH₂OCH₂CH₂—), 3.53 (m, 12H, PhOCH₂CH₂OCH₂CH₂OCH₂CH₂—), 3.37-3.33 (m, 18H, —OCH₃), 1.74 (m, 8H, PhOCH₂CH₂(CH₂)₉CH₃), 1.42 (m, 8H, PhOCH₂CH₂CH₂(CH₂)₈CH₃), 1.39 (s, 3H, —C(CH₂O—)₂CH₃), 1.25 (br, 64H, PhOCH₂CH₂CH₂(CH₂)₈CH₃), 0.87 (t, 12H, PhO(CH₂)₁₁CH₃). ¹³C NMR (500 MHz, CDCl₃) δ 172.3, 165.8, 165.5, 165.1, 160.2, 152.4, 143.0, 142.9, 131.2, 131.2, 124.2, 124.0, 109.1, 109.0, 107.7, 106.5, 72.5, 72.0, 72.0, 70.8, 70.7, 70.7, 70.6, 70.6, 70.6, 69.8, 69.6, 68.9, 68.3, 67.1, 65.7, 63.2, 62.5, 59.1, 46.9, 32.0, 29.7, 29.7, 29.7, 29.6, 29.5, 29.4, 29.2, 26.1, 22.7, 18.1, 14.2. Purity by HPLC: 99+%. MALDI-TOF MS m/z of [M+Na]⁺ calculated for C₁₂₆H₂₁₂O₃₈Na: 2358.0; Found: 2358.3. The ¹H NMR and ¹³C NMR spectra of compound 2S are shown in FIG. 98 and FIG. 99, respectively.

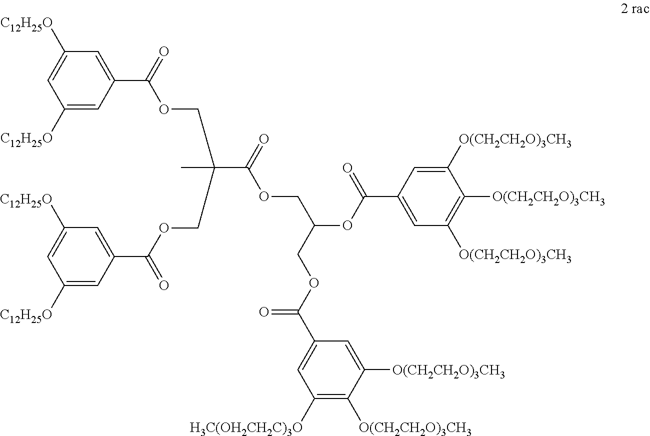

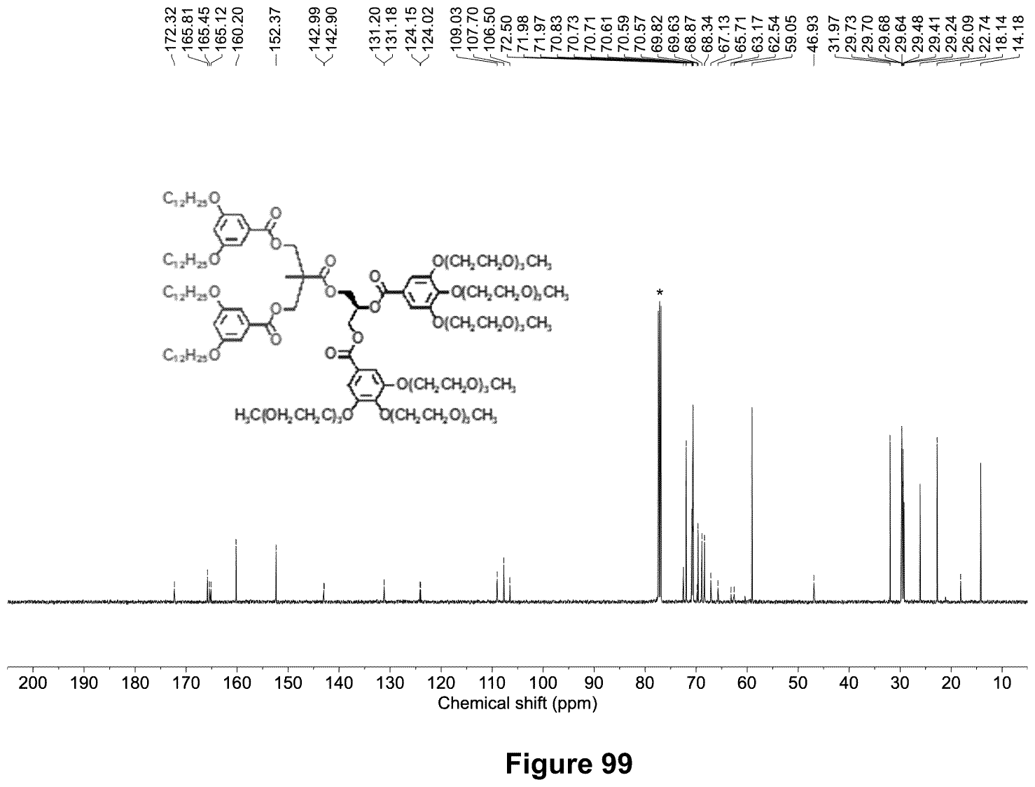

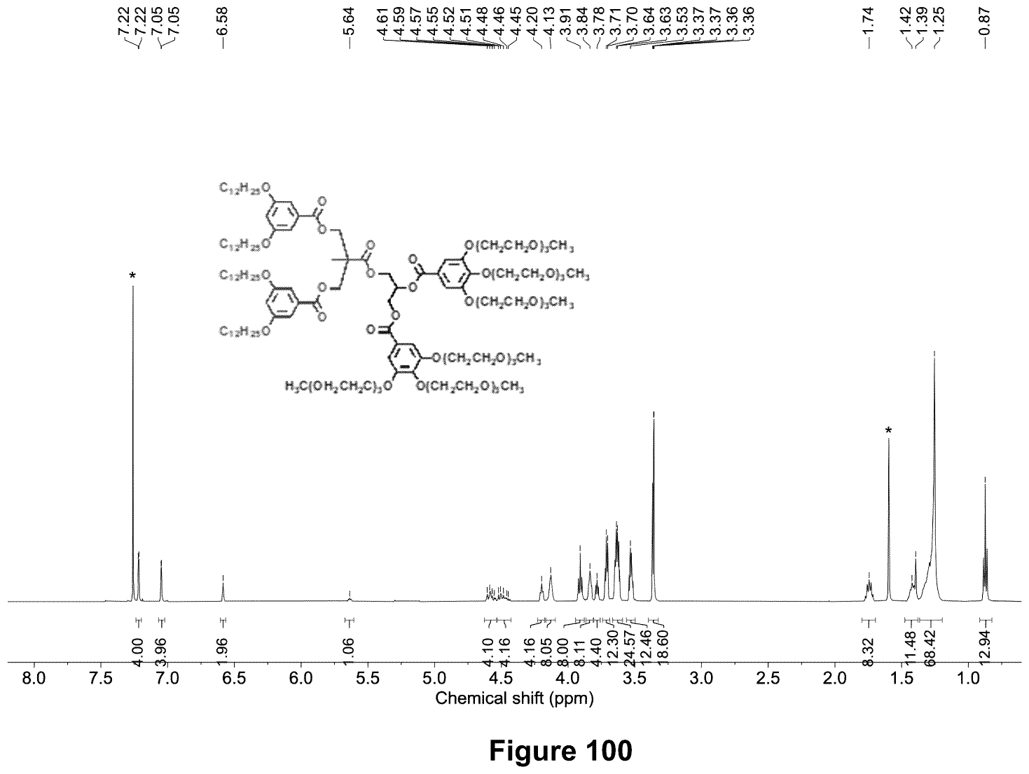

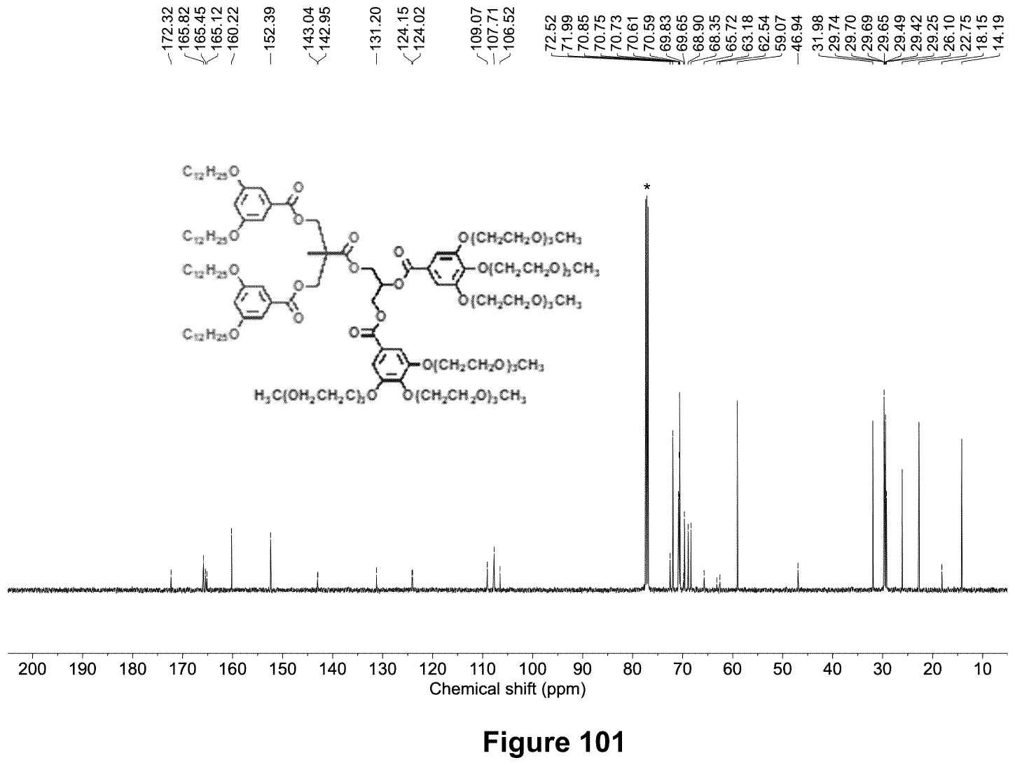

(3,5)-12G1-BMPA-GC-(rac)-(3,4,5)-3EO-G1 (2rac). Compound 2rac was synthesized from (3,5)-12G1-BMPA-GC-(rac)-(1,2)(OH)₂ (27rac, 0.24 g, 0.21 mmol, 1.0 equiv) and (3,4,5)-3EO-G1-COOH (0.29 g, 0.48 mmol, 2.3 equiv) following a procedure similar to that used for the synthesis of 2R. The title compound was obtained as a light-yellow oil (0.38 g, 78%). ¹H NMR (500 MHz, CDCl₃) δ 7.22 (d, 4H, PhH), 7.05 (d, 2H, PhH), 7.05 (d, 2H, PhH), 6.58 (t, 2H, PhH), 5.64 (m, 1H, CH(O—)(CH₂O—)₂), 4.63-4.43 (m, 8H, —C((CH₂O—)₂)CH₃, —COO—CH₂O—CH(O—)- and —CH(O—)—CH₂O—), 4.20 (m, 4H, PhOCH₂CH₂O—), 4.13 (m, 8H, PhOCH₂CH₂O—), 3.91 (t, 8H, PhOCH₂CH₂O—), 3.84 (m, 8H, PhOCH₂(CH₂)₁₀CH₃), 3.78 (t, 4H, PhOCH₂CH₂O—), 3.74-3.69 (m, 12H, PhOCH₂CH₂OCH₂CH₂—), 3.67-3.60 (m, 24H, PhOCH₂CH₂OCH₂CH₂OCH₂CH₂—), 3.53 (m, 12H, PhOCH₂CH₂OCH₂CH₂OCH₂CH₂—), 3.38-3.33 (m, 18H, —OCH₃), 1.74 (m, 8H, PhOCH₂CH₂(CH₂)₉CH₃), 1.42 (m, 8H, PhOCH₂CH₂CH₂(CH₂)₈CH₃), 1.39 (s, 3H, —C(CH₂O—)₂CH₃), 1.25 (br, 64H, PhOCH₂CH₂CH₂(CH₂)₈CH₃), 0.87 (t, 12H, PhO(CH₂)₁₁CH₃). ¹³C NMR (500 MHz, CDCl₃) δ 172.3, 165.8, 165.5, 165.1, 160.2, 152.4, 143.0, 143.0, 131.2, 131.2, 124.2, 124.0, 109.1, 109.1, 107.7, 106.5, 72.5, 72.0, 70.9, 70.8, 70.7, 70.6, 70.6, 69.8, 69.7, 68.9, 68.4, 65.7, 63.2, 62.5, 59.1, 46.9, 32.0, 29.7, 29.7, 29.7, 29.7, 29.5, 29.4, 29.3, 26.1, 22.8, 18.2, 14.2. Purity by HPLC: 99+%. MALDI-TOF MS m/z of [M+Na]⁺ calculated for C₁₂₆H₂₁₂O₃₈Na: 2358.0; Found: 2358.5. The ¹H NMR and ¹³C NMR spectra of compound 2rac are shown in FIG. 100 and FIG. 101, respectively.

### Synthesis of Amphiphilic Glycerol-Based Achiral Janus Dendrimer 3

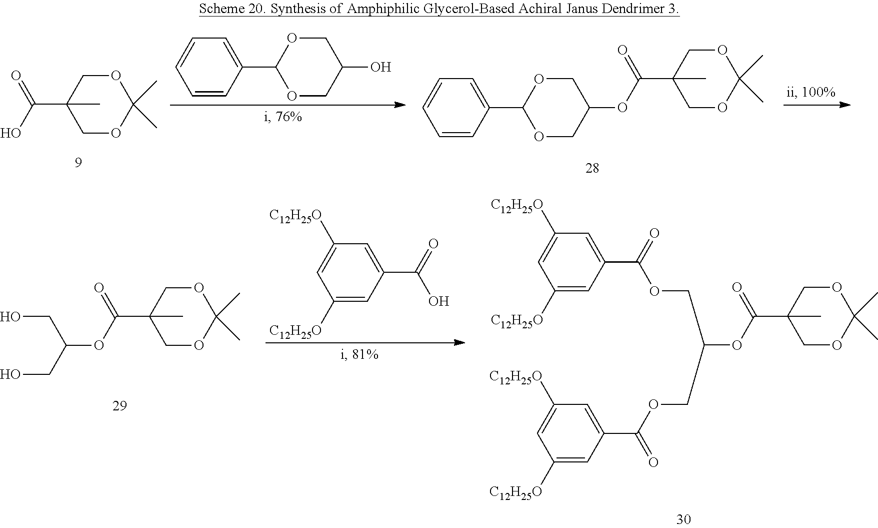

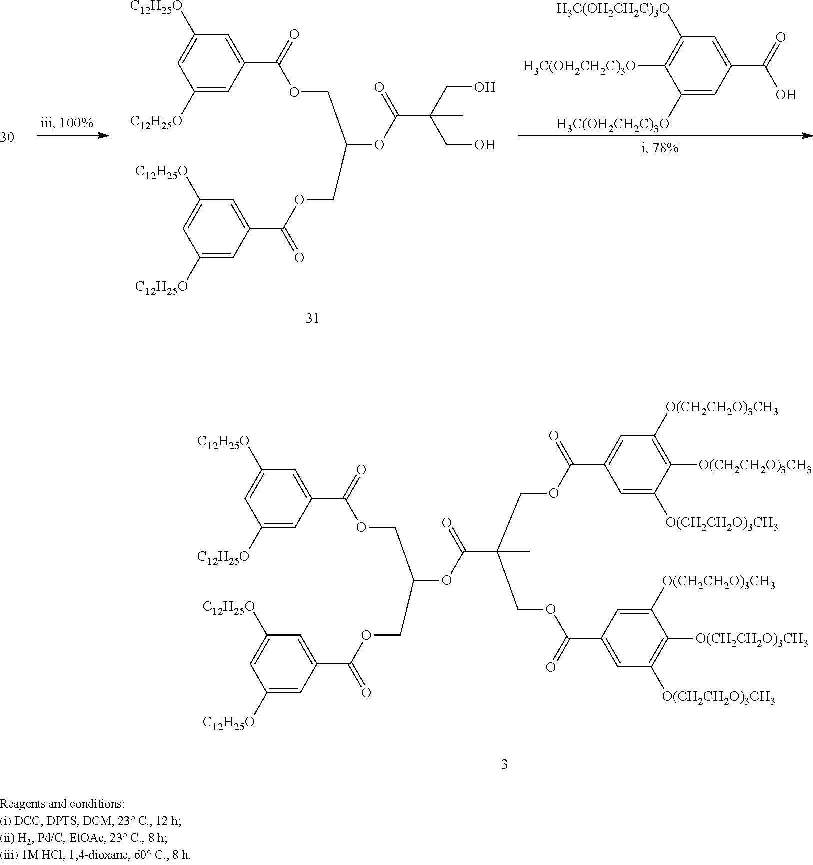

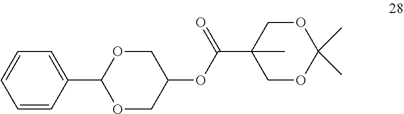

2-Phenyl-1,3-dioxan-5-yl-2,2,5-trimethyl-1,3-dioxane-5-carboxylate (28). 2,2,5-Trimethyl-1,3-dioxane-5-carboxylic acid (9, 1.00 g, 5.74 mmol, 1.0 equiv), 5-hydroxy-2-phenyl-1,3-dioxane (1.04 g, 5.74 mmol, 1.0 equiv) and DPTS (1.79 g, 5.74 mmol, 1.0 equiv) were dissolved in 10 mL dry DCM. DCC (1.78 g, 8.61 mmol, 1.5 equiv) dissolved in 2 mL dry DCM was added in one portion into the above mixture. The reaction mixture was allowed to stir at 23° C. for 12 h. The reaction mixture was filtered to remove the urea, which was washed with DCM carefully. The solvent was then removed under vacuum and the crude product was dissolved in EtOAc. The EtOAc solution was filtered through Celite and the filter cake was washed with EtOAc. The filtrate was collected, concentrated and purified by column chromatography (SiO₂) with Hexane/EtOAc=3/1 to give the title compound as a white solid (1.46 g, 76%). ¹H NMR (500 MHz, CDCl₃) δ 7.49 (m, 2H, PhH), 7.38 (m, 3H, PhH), 5.55 (s, 1H, PhCH(OCH₂)₂—), 4.76 (m, 1H, —(OCH₂)₂—CHOCO—), 4.27 (d, 4H, 2×-OCH₂—), 4.18 (q, 2H, —OCH₂—), 3.73 (d, 2H, —OCH₂—), 1.45 (s, 3H, C(O—)₂(CH₃)CH₃), 1.41 (s, 3H, C(O—)₂(CH₃)CH₃), 1.30 (s, 3H, C(CH₂O—)₂CH₃). ¹³C NMR (500 MHz, CDCl₃) δ 174.2, 138.1, 129.3, 128.4, 126.3, 101.5, 98.2, 68.9, 66.3, 66.0, 42.1, 24.4, 23.1, 18.8. Mp=93° C.

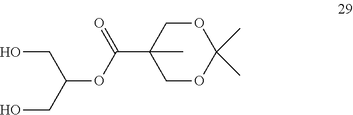

1,3-Dihydroxypropan-2-yl-2,2,5-trimethyl-1,3-dioxane-5-carboxylate (29). Compound 28 (0.78 g, 2.32 mmol) was dissolved in EtOAc (20 mL). Then Pd/C (39 mg, 5 wt %) was added and the flask was evacuated and filled with hydrogen for three times. The mixture was stirred at 23° C. for 8 h under hydrogen atmosphere. The reaction mixture was filtered through Celite and the filter cake was washed with EtOAc. Evaporation of the solvent yielded the title compound as a colorless oil (0.58 g, 100%). ¹H NMR (500 MHz, CDCl₃) δ 5.02 (m, 1H, —(OCH₂)₂—CHOCO—), 4.24 (d, 2H, —OCH₂—), 3.84 (m, 4H, 2×-OCH₂—), 3.70 (d, 2H, OCH₂—), 2.84 (br, 2H, 2×—CH₂OH), 1.45 (s, 3H, C(O—)₂(CH₃)CH₃), 1.36 (s, 3H, C(O—)₂(CH₃)CH₃), 1.09 (s, 3H, C(CH₂O-)₂CH₃). ¹³C NMR (500 MHz, CDCl₃) δ 174.4, 98.6, 75.9, 66.6, 61.8, 42.7, 27.4, 20.0, 17.8.

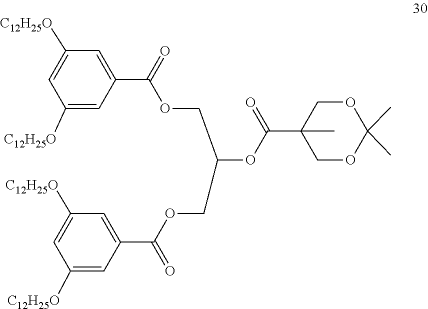

1,3-bis[3,5-bis(dodecyloxy)benzoate]propan-2-yl-2,2,5-trimethyl-1,3-dioxane-5-carboxylate (30). Compound 29 (0.16 g, 0.64 mmol, 1 equiv), (3,5)-12G1-COOH (0.73 g, 1.47 mmol, 2.3 equiv) and DPTS (0.46 g, 1.47 mmol, 2.3 equiv) were dissolved in 8 mL dry DCM. DCC (0.40 g, 1.92 mmol, 3.0 equiv) dissolved in 2 mL dry DCM was added in one portion into the above mixture. The reaction mixture was allowed to stir at 23° C. for 12 h. The reaction mixture was filtered to remove the urea, which was washed with DCM carefully. The filtrate was concentrated and purified by column chromatography (SiO₂) with Hexane/EtOAc=20/1 to give the title compound as a colorless oil (0.58 g, 81%). ¹H NMR (500 MHz, CDCl₃) δ 7.13 (d, 4H, PhH), 6.64 (t, 2H, PhH), 5.59 (m, 1H, —(OCH₂)₂—CHOCO—), 4.61 (q, 2H, —OCH₂—), 4.51 (q, 2H, —OCH₂—), 4.14 (d, 2H, —OCH₂—), 3.95 (t, 8H, PhOCH₂(CH₂)₁₀CH₃), 3.61 (d, 2H, —OCH₂—), 1.77 (m, 8H, PhOCH₂CH₂(CH₂)₉CH₃), 1.44 (m, 8H, PhOCH₂CH₂CH₂(CH₂)₈CH₃), 1.38 (s, 3H, C(O-)₂(CH₃)CH₃), 1.28 (s, 3H, C(O-)₂(CH₃)CH₃), 1.26 (br, 64H, PhOCH₂CH₂CH₂(CH₂)₈CH₃), 1.20 (s, 3H, C(CH₂O—)₂CH₃), 0.88 (t, 12H, PhO(CH₂)₁₁CH₃). ¹³C NMR (500 MHz, CDCl₃) δ 173.5, 166.0, 160.4, 131.2, 107.9, 107.0, 98.2, 69.7, 68.4, 65.9, 62.8, 42.0, 32.0, 29.8, 29.8, 29.7, 29.7, 29.5, 29.5, 29.3, 26.2, 23.7, 23.6, 22.8, 18.7, 14.2.

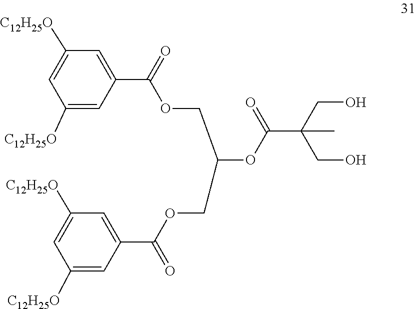

1,3-bis[3,5-bis(dodecyloxy)benzoate]propan-2-yl-3-hydroxy-2-(hydroxymethyl)-2-methylpropanoate (31). Compound 30 (0.43 g, 0.36 mmol) was dissolved in 10 mL 1,4-dioxane. Then HCl (2 M, 2.0 mL, 4.0 mmol) was added. The mixture was stirred at 60° C. for 8 h. The reaction mixture was extracted by DCM (3×20 mL) and the organic phase was dried over anhydrous magnesium sulfate. Filtration and evaporation of the solvent yielded the title compound as a colorless oil (0.42 g, 100%). ¹H NMR (500 MHz, CDCl₃) δ 7.13 (d, 4H, PhH), 6.64 (t, 2H, Phi), 5.60 (m, 1H, —(OCH₂)₂—CHOCO—), 4.71 (q, 2H, —OCH₂—), 4.51 (q, 2H, —OCH₂—), 3.95 (t, 8H, PhOCH₂(CH₂)₁₀CH₃), 3.82 (q, 2H, —OCH₂—), 3.70 (q, 2H, —OCH₂—), 2.75 (t, 2H, 2×-CH₂OH), 1.77 (m, 8H, PhOCH₂CH₂(CH₂)₉CH₃), 1.44 (m, 8H, PhOCH₂CH₂CH₂(CH₂)₈CH₃), 1.26 (br, 64H, PhOCH₂CH₂CH₂(CH₂)₈CH₃), 1.06 (s, 3H, C(CH₂O—)₂CH₃), 0.88 (t, 12H, PhO(CH₂)₁₁CH₃). ¹³C NMR (500 MHz, CDCl₃) δ 175.0, 166.3, 160.4, 131.0, 107.9, 107.0, 70.1, 68.5, 68.0, 67.6, 62.9, 49.9, 32.0, 29.8, 29.7, 29.7, 29.7, 29.5, 29.5, 29.3, 26.1, 22.8, 17.2, 14.2.

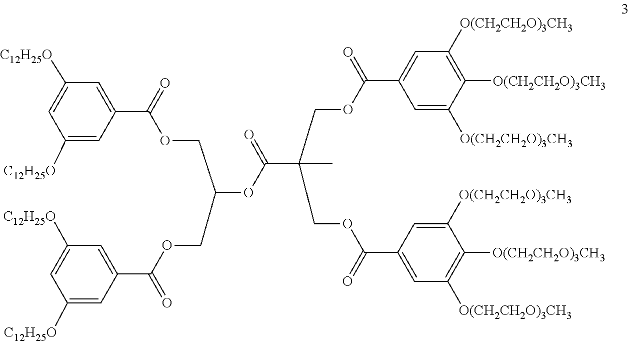

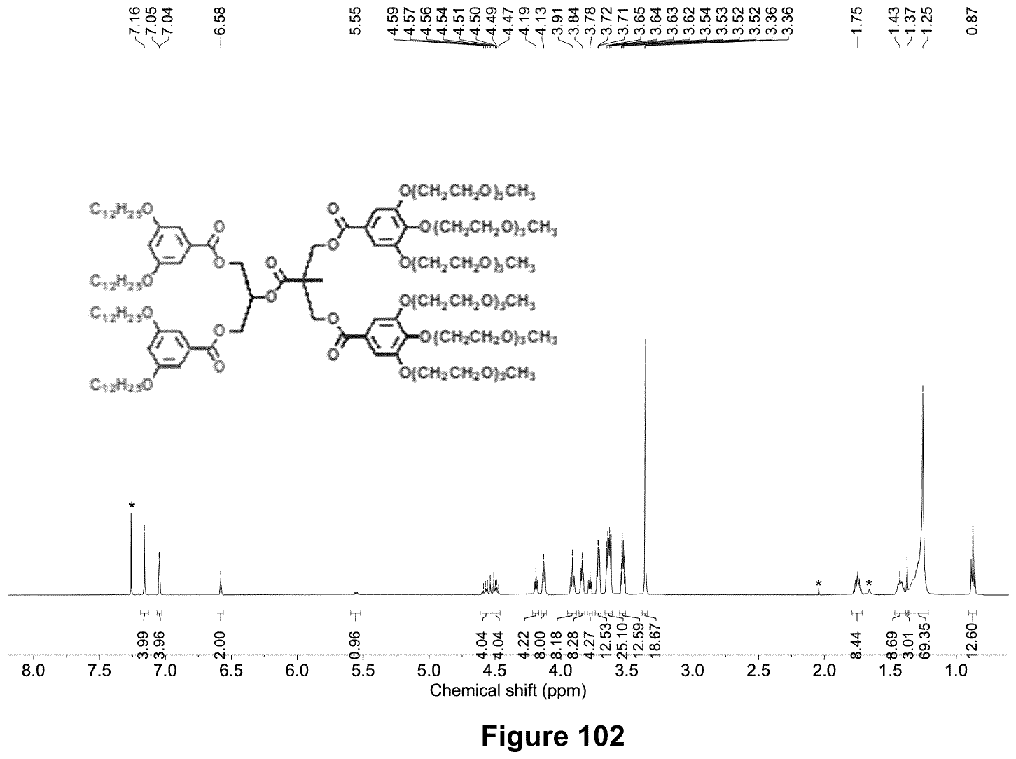

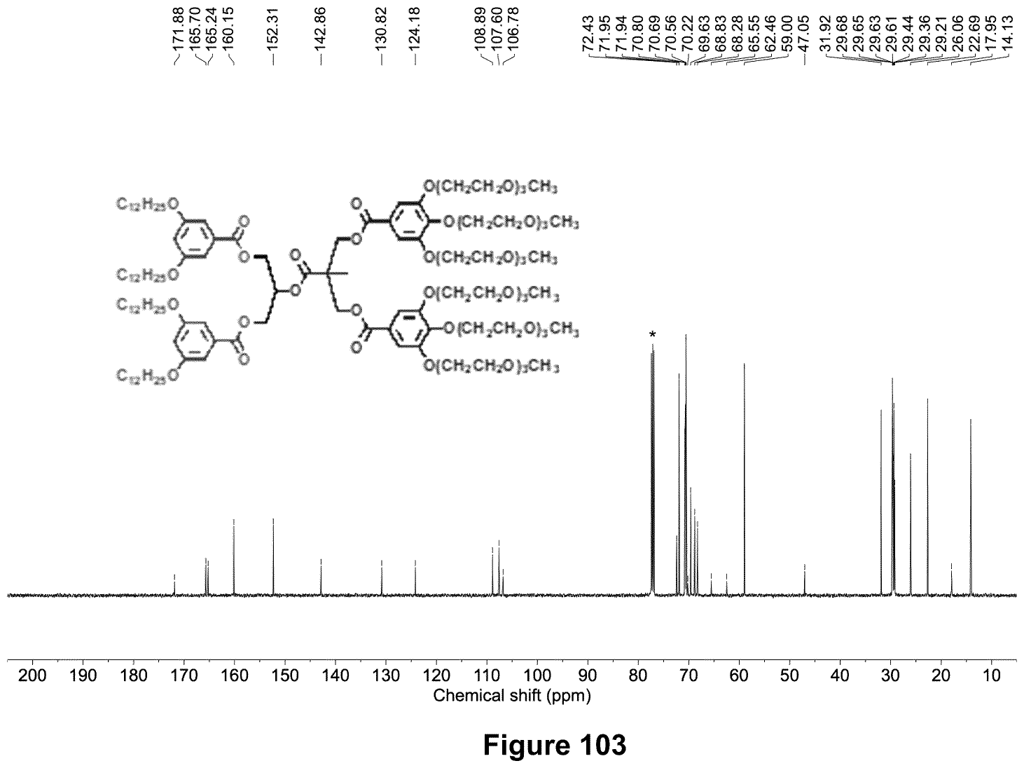

(3,5)-12G1-GC-(achiral)-BMPA-(3,4,5)-3EO-G2 (3). Compound 31 (0.21 g, 0.18 mmol, 1 equiv), (3,4,5)-3EO-G1-COOH (0.25 g, 0.41 mmol, 2.3 equiv) and DPTS (0.13 g, 0.41 mmol, 2.3 equiv) were dissolved in 6 mL dry DCM. DCC (0.11 g, 0.54 mmol, 3.0 equiv) dissolved in 2 mL dry DCM was added in one portion into the above mixture. The reaction mixture was allowed to stir at 23° C. for 12 h. The reaction mixture was filtered to remove the urea, which was washed with DCM carefully. The filtrate was concentrated and purified by column chromatography (SiO₂) with EtOAc/MeOH=20/1 as the eluent to give the title compound as a light-yellow oil (0.31 g, 78%). ¹H NMR (500 MHz, CDCl₃) δ 7.16 (s, 4H, PhH), 7.05 (d, 4H, PhH), 6.58 (t, 2H, PhH), 5.55 (m, 1H, CH(O—)(CH₂O—)₂), 4.61-4.45 (m, 8H, —C((CH₂O—)₂)CH₃ and CH(O—)(CH₂O—)₂), 4.19 (m, 4H, PhOCH₂CH₂O—), 4.13 (m, 8H, PhOCH₂CH₂O—), 3.91 (t, 8H, PhOCH₂CH₂O—), 3.84 (m, 8H, PhOCH₂(CH₂)₁₀CH₃), 3.78 (t, 4H, PhOCH₂CH₂O—), 3.74-3.69 (m, 12H, PhOCH₂CH₂OCH₂CH₂—), 3.67-3.61 (m, 24H, PhOCH₂CH₂OCH₂CH₂OCH₂CH₂—), 3.53 (m, 12H, PhOCH₂CH₂OCH₂CH₂OCH₂CH₂—), 3.37-3.34 (m, 18H, —OCH₃), 1.75 (m, 8H, PhOCH₂CH₂(CH₂)₉CH₃), 1.43 (m, 8H, PhOCH₂CH₂CH₂(CH₂)₈CH₃), 1.37 (s, 3H, —C(CH₂O—)₂CH₃), 1.25 (br, 64H, PhOCH₂CH₂CH₂(CH₂)₈CH₃), 0.87 (t, 12H, PhO(CH₂)₁₁CH₃). ¹³C NMR (500 MHz, CDCl₃) δ 171.9, 165.7, 165.2, 160.2, 152.3, 142.9, 130.8, 124.2, 108.9, 107.6, 106.8, 72.4, 72.0, 71.9, 70.8, 70.7, 70.6, 70.2, 69.6, 68.8, 68.3, 65.6, 62.5, 59.0, 47.1, 31.9, 29.7, 29.7, 29.6, 29.6, 29.4, 29.4, 29.2, 26.1, 22.7, 18.0, 14.1. Purity by HPLC: 99+%. MALDI-TOF MS m/z of [M+Na]⁺ calculated for C₁₂₆H₂₁₂O₃₈Na: 2358.0; Found: 2358.1. The ¹H NMR and ¹³C NMR spectra of compound 3 are shown in FIG. 102 and FIG. 103, respectively.

### Synthesis of Amphiphilic Glycerol-Based Achiral Janus Dendrimer 4

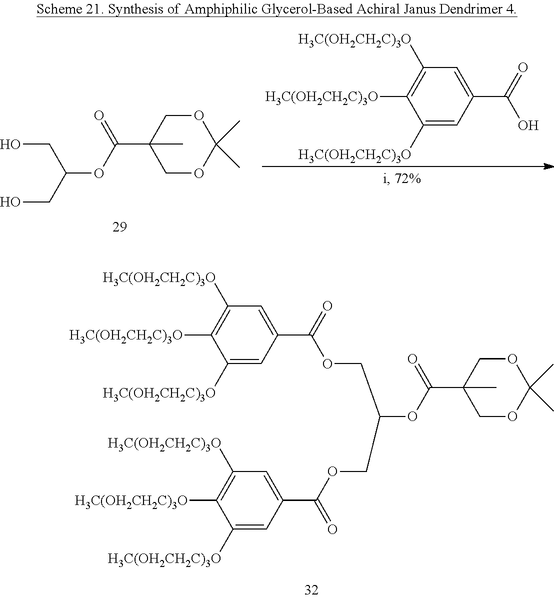

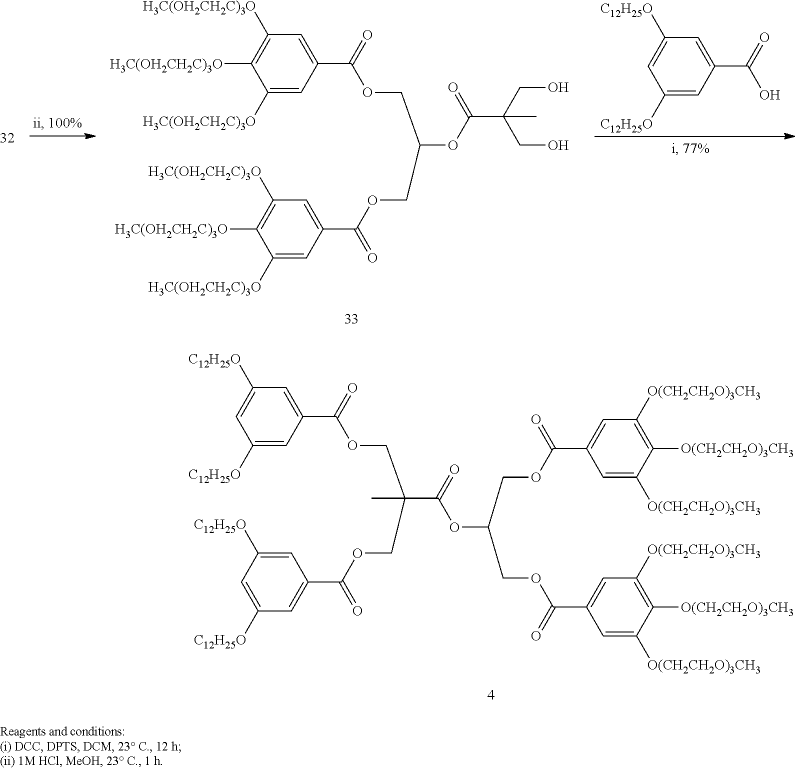

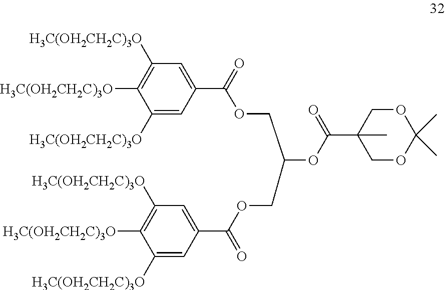

1,3-bis[3,4,5-tris(methyl triethylene glycol)benzoate]propan-2-yl-2,2,5-trimethyl-1,3-dioxane-5-carboxylate (32). Compound 29 (0.15 g, 0.60 mmol, 1 equiv), (3,4,5)-3EO-G1-COOH (0.84 g, 1.38 mmol, 2.3 equiv) and DPTS (0.43 g, 1.38 mmol, 2.3 equiv) were dissolved in 8 mL dry DCM. DCC (0.37 g, 1.80 mmol, 3.0 equiv) dissolved in 2 mL dry DCM was added in one portion into the above mixture. The reaction mixture was allowed to stir at 23° C. for 12 h. The reaction mixture was filtered to remove the urea, which was washed with DCM carefully. The filtrate was concentrated and purified by column chromatography (SiO₂) with DCM/MeOH=20/1 to give the title compound as a colorless oil (0.61 g, 72%). ¹H NMR (500 MHz, CDCl₃) δ 7.26 (s, 4H, PhH), 5.59 (m, 1H, CH(O—)(CH₂O—)₂), 4.57 (q, 2H, —OCH₂—), 4.49 (q, 2H, —OCH₂—), 4.21 (t, 4H, PhOCH₂CH₂O—), 4.17 (t, 8H, PhOCH₂CH₂O—), 4.14 (d, 2H, —OCH₂—), 3.86 (t, 8H, PhOCH₂CH₂O—), 3.79 (t, 4H, PhOCH₂CH₂O—), 3.75-3.69 (m, 12H, PhOCH₂CH₂OCH₂CH₂—), 3.67-3.61 (m, 24H, PhOCH₂CH₂OCH₂CH₂OCH₂CH₂—), 3.60 (d, 2H, —OCH₂—), 3.53 (m, 12H, PhOCH₂CH₂OCH₂CH₂OCH₂CH₂—), 3.36 (ds, 18H, —OCH₃), 1.37 (s, 3H, C(O—)₂(CH₃)CH₃), 1.21 (s, 3H, C(O—)₂(CH₃)CH₃), 1.12 (s, 3H, —C(CH₂O—)₂CH₃). ¹³C NMR (500 MHz, CDCl₃) δ 173.3, 165.4, 152.3, 142.8, 124.0, 109.0, 97.9, 72.3, 71.8, 70.7, 70.6, 70.4, 70.4, 69.6, 69.5, 68.8, 65.7, 62.6, 58.9, 41.9, 24.6, 22.3, 18.4.

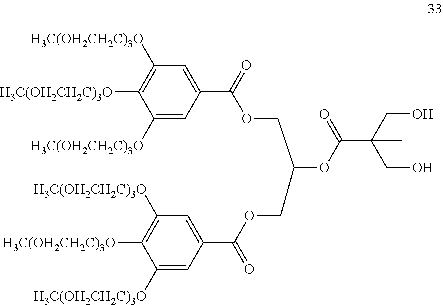

1,3-bis[3,4,5-tris(methyl triethylene glycol)benzoate]propan-2-yl-3-hydroxy-2-(hydroxymethyl)-2-methylpropanoate (33). Compound 32 (0.20 g, 0.14 mmol) was dissolved in 10 mL MeOH. Then HCl (1 M, 2.0 mL, 2.0 mmol) was added. The mixture was stirred at 23° C. for 1 h. The reaction mixture was extracted by DCM (3×20 mL) and the organic phase was dried over anhydrous magnesium sulfate. Filtration and evaporation of the solvent yielded the title compound as a colorless oil (0.19 g, 100%). ¹H NMR (500 MHz, CDCl₃) δ 7.29 (s, 4H, PhH), 5.60 (m, 1H, CH(O—)(CH₂O—)₂), 4.66 (q, 2H, —OCH₂—), 4.44 (q, 2H, —OCH₂—), 4.22 (t, 4H, PhOCH₂CH₂O—), 4.19 (t, 8H, PhOCH₂CH₂O—), 3.86 (t, 8H, PhOCH₂CH₂O—), 3.81 (q, 2H, —OCH₂—), 3.79 (t, 4H, PhOCH₂CH₂O—), 3.75-3.68 (m, 14H, PhOCH₂CH₂OCH₂CH₂— and —OCH₂—), 3.67-3.60 (m, 24H, PhOCH₂CH₂OCH₂CH₂OCH₂CH₂—), 3.53 (m, 12H, PhOCH₂CH₂OCH₂CH₂OCH₂CH₂—), 3.36 (ds, 18H, —OCH₃), 3.06 (t, 2H, 2×-CH₂OH), 1.07 (s, 3H, —C(CH₂O—)₂CH₃). ¹³C NMR (500 MHz, CDCl₃) δ 174.6, 165.6, 152.3, 143.0, 124.0, 109.2, 72.3, 71.8, 71.8, 70.7, 70.6, 70.5, 70.5, 70.4, 69.7, 69.5, 68.8, 62.7, 58.9, 49.7, 17.2.

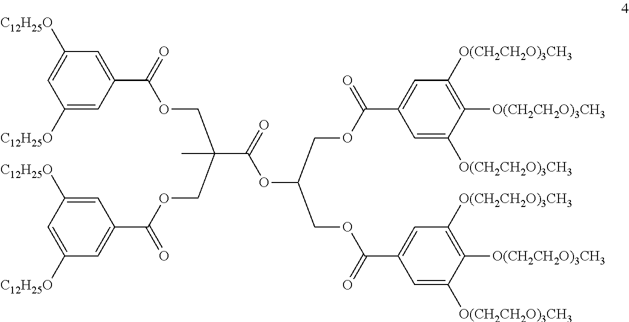

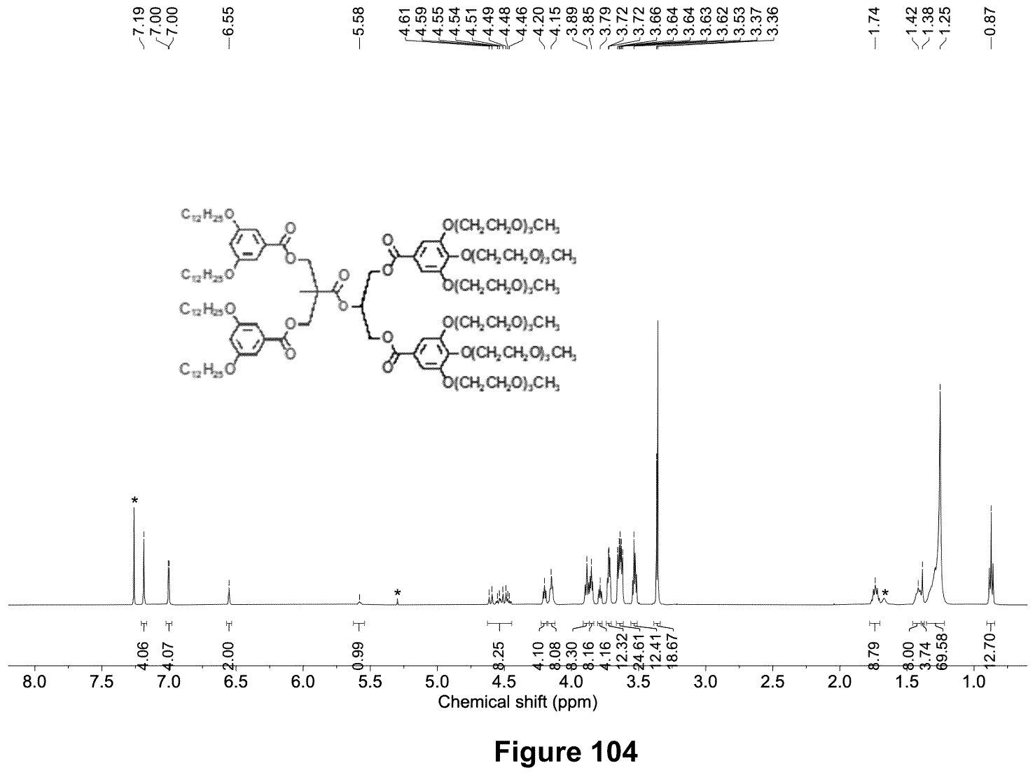

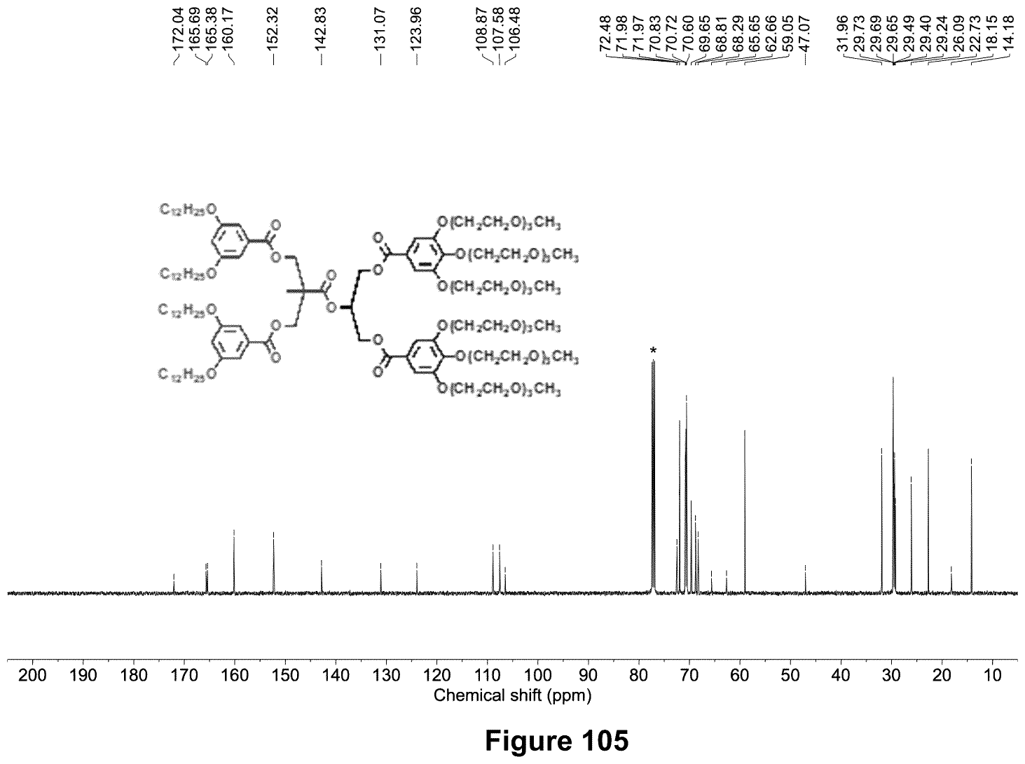

(3,5)-12G2-BMPA-GC-(achiral)-(3,4,5)-3EO-G1 (4). Compound 33 (0.18 g, 0.13 mmol, 1 equiv), (3,5)-12G1-COOH (0.15 g, 0.30 mmol, 2.3 equiv) and DPTS (94 mg, 0.30 mmol, 2.3 equiv) were dissolved in 4 mL dry DCM. DCC (80 mg, 0.39 mmol, 3.0 equiv) dissolved in 2 mL dry DCM was added in one portion into the above mixture. The reaction mixture was allowed to stir at 23° C. for 12 h. The reaction mixture was filtered to remove the urea, which was washed with DCM carefully. The filtrate was concentrated and purified by column chromatography (SiO₂) with DCM/MeOH=20/1 to give the title compound as a colorless oil (0.23 g, 77%). ¹H NMR (500 MHz, CDCl₃) δ 7.19 (s, 4H, PhH), 7.00 (d, 4H, PhH), 6.55 (t, 2H, PhH), 5.58 (m, 1H, CH(O—)(CH₂O—)₂), 4.63-4.43 (m, 8H, —C((CH₂O—)₂)CH₃ and CH(O—)(CH₂O—)₂), 4.20 (t, 4H, PhOCH₂CH₂O—), 4.15 (m, 8H, PhOCH₂CH₂O—), 3.89 (t, 8H, PhOCH₂CH₂O—), 3.85 (t, 8H, PhOCH₂(CH₂)₁₀CH₃), 3.79 (t, 4H, PhOCH₂CH₂O—), 3.74-3.70 (m, 12H, PhOCH₂CH₂OCH₂CH₂—), 3.68-3.60 (m, 24H, PhOCH₂CH₂OCH₂CH₂OCH₂CH₂—), 3.53 (m, 12H, PhOCH₂CH₂OCH₂CH₂OCH₂CH₂—), 3.37-3.34 (m, 18H, —OCH₃), 1.74 (m, 8H, PhOCH₂CH₂(CH₂)₉CH₃), 1.42 (m, 8H, PhOCH₂CH₂CH₂(CH₂)₈CH₃), 1.38 (s, 3H, —C(CH₂O-)₂CH₃), 1.25 (br, 64H, PhOCH₂CH₂CH₂(CH₂)₈CH₃), 0.87 (t, 12H, PhO(CH₂)₁₁CH₃). ¹³C NMR (500 MHz, CDCl₃) δ 172.0, 165.7, 165.4, 160.2, 152.3, 142.8, 131.1, 124.0, 108.9, 107.6, 106.5, 72.5, 72.0, 72.0, 70.8, 70.7, 70.6, 69.7, 68.8, 68.3, 65.7, 62.7, 59.1, 47.1, 32.0, 29.7, 29.7, 29.7, 29.5, 29.4, 29.2, 26.1, 22.7, 18.2, 14.2. Purity by HPLC: 99+%. MALDI-TOF MS m/z of [M+Na]+calculated for C₁₂₆H₂₁₂O₃₈Na: 2358.0; Found: 2358.2. The ¹H NMR and ¹³C NMR spectra of compound 4 are shown in FIG. 104 and FIG. 105, respectively.
DLS Data of the Assemblies from Glycerol-Based JDs

TABLE 25
Size of assemblies from glycerol-based JDs in library 1 at different concentrations
of JDs in water (Size distribution is recorded with intensity).

| Concentration | 1R | | 1S | | 1rac | | 1R:1S - 1:3 | | 1R:1S - 1:1 | | 1R:1S - 3:1 | |
|---|---|---|---|---|---|---|---|---|---|---|---|---|
| of JD | Dₕ | | Dₕ | | Dₕ | | Dₕ | | Dₕ | | Dₕ | |
| (mg · mL⁻¹) | (nm) | PDI | (nm) | PDI | (nm) | PDI | (nm) | PDI | (nm) | PDI | (nm) | PDI |
| 0.5 | 233 | 0.326 | 229 | 0.438 | 281 | 0.240 | 179 | 0.421 | 184 | 0.271 | 266 | 0.376 |
| 0.4 | 178 | 0.228 | 184 | 0.258 | 174 | 0.275 | 181 | 0.268 | 171 | 0.274 | 164 | 0.246 |
| 0.3 | 148 | 0.190 | 147 | 0.186 | 153 | 0.203 | 144 | 0.226 | 147 | 0.236 | 153 | 0.180 |
| 0.25 | 140 | 0.202 | 142 | 0.203 | 142 | 0.238 | 138 | 0.207 | 143 | 0.214 | 134 | 0.186 |
| 0.2 | 135 | 0.175 | 135 | 0.195 | 136 | 0.216 | 139 | 0.197 | 126 | 0.201 | 127 | 0.234 |
| 0.15 | 124 | 0.195 | 124 | 0.190 | 128 | 0.232 | 116 | 0.206 | 119 | 0.247 | 117 | 0.210 |
| 0.1 | 110 | 0.197 | 100 | 0.223 | 123 | 0.268 | 94 | 0.243 | 96 | 0.269 | 110 | 0.257 |
| 0.05 | 87 | 0.336 | 86 | 0.359 | 109 | 0.277 | 89 | 0.401 | 78 | 0.368 | 83 | 0.334 |
| 0.025 | 82 | 0.455 | 80 | 0.446 | 86 | 0.398 | 84 | 0.502 | 76 | 0.410 | 74 | 0.451 |

TABLE 26
Size of assemblies from glycerol-based JDs in library 2 at different concentrations
of JDs in water (Size distribution is recorded with intensity).

| Concentration | 2R | | 2S | | 2rac | | 2R:2S - 1:3 | | 2R:2S - 1:1 | | 2R:2S - 3:1 | |
|---|---|---|---|---|---|---|---|---|---|---|---|---|
| of JD | Dₕ | | Dₕ | | Dₕ | | Dₕ | | Dₕ | | Dₕ | |
| (mg · mL⁻¹) | (nm) | PDI | (nm) | PDI | (nm) | PDI | (nm) | PDI | (nm) | PDI | (nm) | PDI |
| 0.5 | 319 | 0.497 | 288 | 0.295 | 412 | 0.449 | 304 | 0.511 | 324 | 0.271 | 390 | 0.376 |
| 0.4 | 180 | 0.263 | 165 | 0.205 | 181 | 0.283 | 156 | 0.170 | 165 | 0.274 | 166 | 0.246 |
| 0.3 | 152 | 0.227 | 153 | 0.235 | 155 | 0.225 | 144 | 0.295 | 144 | 0.236 | 147 | 0.180 |
| 0.25 | 139 | 0.186 | 132 | 0.223 | 147 | 0.211 | 134 | 0.192 | 138 | 0.214 | 133 | 0.186 |
| 0.2 | 134 | 0.242 | 128 | 0.203 | 139 | 0.260 | 125 | 0.189 | 122 | 0.201 | 117 | 0.234 |
| 0.15 | 125 | 0.257 | 119 | 0.212 | 129 | 0.236 | 116 | 0.223 | 117 | 0.247 | 111 | 0.210 |
| 0.1 | 111 | 0.258 | 112 | 0.291 | 122 | 0.277 | 113 | 0.359 | 110 | 0.269 | 103 | 0.213 |
| 0.05 | 108 | 0.368 | 107 | 0.422 | 119 | 0.365 | 98 | 0.359 | 92 | 0.280 | 89 | 0.361 |
| 0.025 | 107 | 0.416 | 106 | 0.465 | 118 | 0.361 | 81 | 0.419 | 87 | 0.309 | 89 | 0.439 |

TABLE 27
Size of assemblies from glycerol-based achiral JDs 3
and 4 at different concentrations of JDs in water
(Size distribution is recorded with intensity).

| Comcentration | | | | |
|---|---|---|---|---|
| of JD | 3 | | 4 | |
| (mg · mL⁻¹) | Dₕ (nm) | PDI | Dₕ (nm) | PDI |
| 1.0 | 266 | 0.233 | 284 | 0.291 |
| 0.75 | 224 | 0.236 | 210 | 0.233 |
| 0.5 | 162 | 0.152 | 166 | 0.183 |
| 0.25 | 144 | 0.153 | 132 | 0.180 |
| 0.1 | 123 | 0.183 | 119 | 0.136 |
| 0.05 | 97 | 0.241 | 102 | 0.266 |
| 0.025 | 72 | 0.272 | 100 | 0.391 |

Statistical Analysis of Number of Vesicle Layers of DSs Self-Assembled from Glycerol-Based JDs

TABLE 28
Number of vesicle layers for each molecule of library 1.

| | | |
|---|---|---|
| Glycerol | Sample | Number of vesicle layers |

| JDs | quantity | 1 | 2 | 3 | 4 | 5 | 6 |
|---|---|---|---|---|---|---|---|
| 1rac | 20 | 7 | 6 | 2 | 1 | 1 | 3 |
| 1R | 82 | 61 | 15 | 4 | 1 | 1 | 0 |
| 1S | 40 | 23 | 9 | 3 | 4 | 1 | 0 |

TABLE 29
Number of vesicle layers for each molecule of library 2.

| Glycerol | Sample | Number of vesicle layers | | | | | | | | | | | |
|---|---|---|---|---|---|---|---|---|---|---|---|---|---|
| JDs | quantity | 1 | 2 | 3 | 4 | 5 | 6 | 7 | 8 | 9 | 10 | 11 | 12 |
| 2rac | 306 | 29 | 31 | 40 | 62 | 54 | 27 | 25 | 14 | 14 | 4 | 4 | 2 |
| 2R | 216 | 202 | 2 | 5 | 1 | 3 | 2 | 0 | 0 | 0 | 0 | 1 | 0 |
| 2S | 92 | 63 | 13 | 9 | 1 | 0 | 0 | 1 | 1 | 0 | 3 | 1 | 0 |

TABLE 30
Comparison of the distribution parameters, skewness and excess kurtosis.

| Glycerol JDs | Sample quantity | Skewness | Excess Kurtosis |
|---|---|---|---|
| 1rac | 20 | 0.705 | −1.623 |
| 1R | 82 | 1.977 | 3.932 |
| 1S | 40 | 1.687 | 2.797 |
| 2rac | 306 | 0.585 | −0.423 |
| 2R | 216 | 3.313 | 10.980 |
| 2S | 92 | 3.029 | 9.502 |

TABLE 31
Distribution parameters of 1rac.

| 1rac | | Statistic | Std. Error |
|---|---|---|---|
| Mean | | 3.333 | 1.054 |
| 95% Confidence | Lower Bound | 0.624 | |
| Interval for mean | Upper Bound | 6.043 | |
| 5% Trimmed Mean | | 3.259 | |
| Median | | 2.500 | |
| Mode | | 1 | |
| Variance | | 6.667 | |
| Std. Deviation | | 2.582 | |
| Minimum | | 1.00 | |
| Maximum | | 7.00 | |
| Range | | 6.00 | |
| Interquartile Range | | 5.25 | |
| Skewness | | 0.705 | 0.845 |
| Excess Kurtosis | | −1.623 | 1.741 |

TABLE 32
Test of normality for 1rac.

| | |
|---|---|
| Kolmogorov-Smirnov | Shapiro-Wilk |

| | Statistic | df | Sig. | Statistic | df | Sig. |
|---|---|---|---|---|---|---|
| 1rac | 0.218 | 6 | 0.200* | 0.859 | 6 | 0.184 |

*This is a lower bound of the true significance.

TABLE 33
Distribution parameters of 2rac.

| 2rac | | Statistic | Std. Error |
|---|---|---|---|
| Mean | | 25.5 | 5.601 |
| 95% Confidence | Lower Bound | 13.172 | |
| Interval for mean | Upper Bound | 37.828 | |
| 5% Trimmed Mean | | 24.778 | |
| Median | | 26.000 | |
| Mode | | 4 | |
| Variance | | 376.455 | |
| Std. Deviation | | 19.402 | |
| Minimum | | 2.00 | |
| Maximum | | 62.00 | |
| Range | | 60.00 | |
| Interquartile Range | | 31.25 | |
| Skewness | | 0.585 | 0.637 |
| Excess Kurtosis | | −0.423 | 1.232 |

TABLE 34
Test of normality for 2rac.

| | |
|---|---|
| Kolmogorov-Smirnov | Shapiro-Wilk |

| | Statistic | df | Sig. | Statistic | df | Sig. |
|---|---|---|---|---|---|---|
| 2rac | 0.140 | 12 | 0.200* | 0.930 | 12 | 0.383 |

* This is a lower bound of the true significance.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An ionizable amphiphilic Janus dendrimer having the structure of Formula (I): 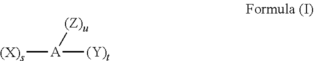
wherein:
A is a polyvalent group comprising at least one selected from the group consisting of: 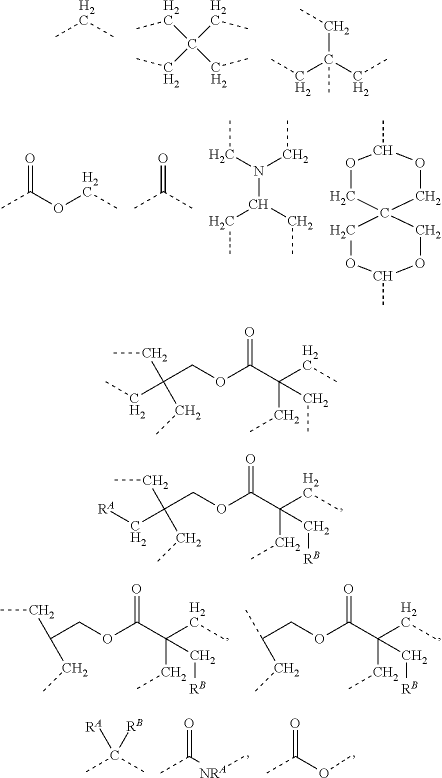
and any combination thereof;
dashed lines represent a binding site of one of X, Y, or Z;
X is a hydrophilic group comprising at least one amine;
Y is a lipophilic group comprising at least one C₁-C₃₀-alkyl chain;
Z comprises at least one selected from the group consisting of ethylene glycol, diethylene glycol, triethylene glycol, and polyethylene glycol chain;
R^{A} and R^{B} are independently selected from the group consisting of hydrogen, halide, hydroxy, C₁-C₃₀-alkyl, C₁-C₃₀-alkyl halide, C₁-C₃₀-alkoxy, C₁-C₃₀-alkoxy halide, and any combination thereof,
s is an integer from 0 to 5;
t is an integer from 0 to 5;
u is an integer from 0 to 4; and
the sum of s, t, and u is equal to the valency of A.

2. The ionizable amphiphilic Janus dendrimer of claim 1, wherein the ionizable amphiphilic Janus dendrimer having the structure of Formula (I) is an ionizable amphiphilic Janus dendrimer having the structure of Formula (II): 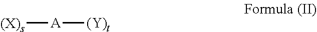
wherein:
A is a polyvalent group comprising at least one selected from the group consisting of 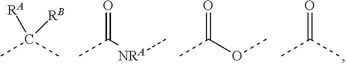
and any combination thereof,
dashed lines represent a binding site of one of X and Y;
R^{A} and R^{B} are independently selected from the group consisting of hydrogen, halide, hydroxy, C₁-C₃₀-alkyl, C₁-C₃₀-alkyl halide, C₁-C₃₀-alkoxy, C₁-C₃₀-alkoxy halide, and any combination thereof,
s is an integer from 1 to 5;
t is an integer from 1 to 5; and
the sum of s and t is equal to the valency of A.

3. The ionizable amphiphilic Janus dendrimer of claim 1, wherein Y is a lipophilic group comprising at least two C₁-C₃₀-alkyl chains having differing numbers of carbon atoms.

4. The ionizable amphiphilic Janus dendrimer of claim 1, wherein A is represented by 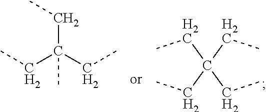
and
s and t are each 2.

5. The ionizable amphiphilic Janus dendrimer of claim 1, wherein A is represented by 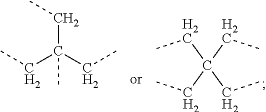
t is 2;
s is 1; and
u is 0 or 1.

6. The ionizable amphiphilic Janus dendrimer of claim 1, wherein A is represented by 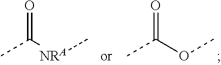
and
s and t are each 1.

7. The ionizable amphiphilic Janus dendrimer of claim 1, wherein each occurrence of X is independently selected from the group consisting of: 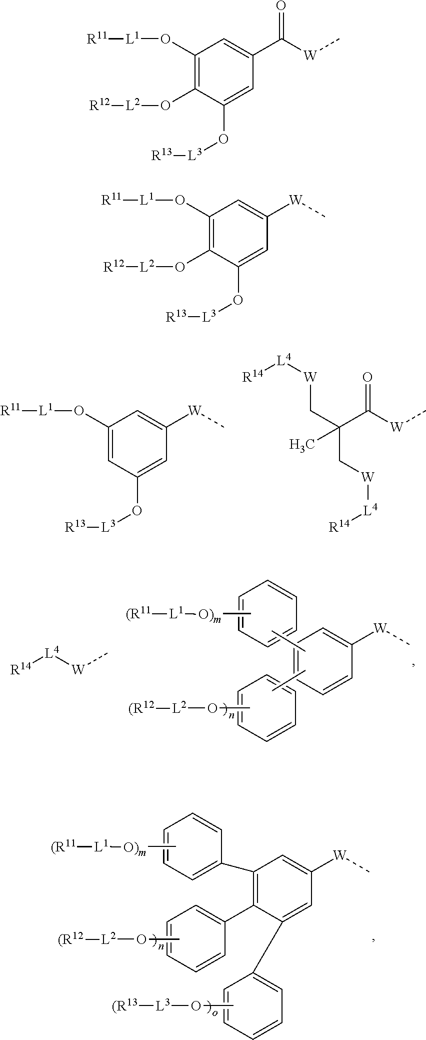
and any combination thereof;
wherein dashed lines indicate the connection to A;
each occurrence of W is independently selected from the group consisting of C═O, C(R^{W})(R^{W}), NR^{W}, O, and S;
wherein each occurrence of R^{W} is independently selected from the group consisting of hydrogen, halide, hydroxy, alkyl, alkyl halide, aryl, aryl halide, alkoxy, alkoxy halide, and any combination thereof; and
each occurrence of L¹, L², L³, and L⁴ is independently a covalent bond or a divalent linking group selected from the group consisting of alkylene, cycloalkylene, heteroalkylene, heterocycloalkylene, alkenylene, alkynylene, arylene, heteroarylene, silyl, amine, amide, ester, ether, carbonyl, carbamate, thioether, thioester, disulfide, hydrazine, urea, thiourea, phosphate, poly(alkyl ether), heteroatom, and any combinations thereof;
each occurrence of R¹¹, R¹², R¹³, and R¹⁴ is independently selected from the group consisting of hydrogen, deuterium, alkyl, aryl, heteroaryl, cycloalkyl, alkoxy, phenoxy, amine, heterocycloalkyl, carbonyl, and any combinations thereof; and
each occurrence of m, n, o is independently an integer from 1 to 5.

8. The ionizable amphiphilic Janus dendrimer of claim 7, wherein at least one occurrence of R¹¹, R¹², R¹³, or R¹⁴ is —C(O)(CH₂)ₘ—N(R¹)(R²)
each occurrence of m is independently an integer from 1 to 10; and
each occurrence of R¹ and R² is independently selected from the group consisting of hydrogen, deuterium, alkyl, aryl, cycloalkyl, amine, heterocycloalkyl, carbonyl, and any combinations thereof; wherein R¹ and R² may together form a ring.

9. The ionizable amphiphilic Janus dendrimer of claim 7, wherein each occurrence of R¹¹, R¹², R¹³, and R¹⁴ is independently selected from the group consisting of: 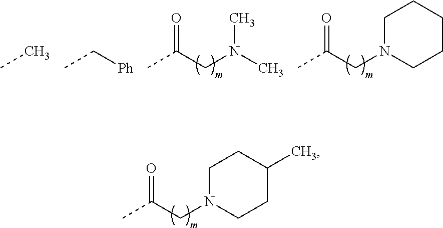
and any combination thereof,
wherein each occurrence of m is independently an integer from 1 to 10.

10. The ionizable amphiphilic Janus dendrimer of claim 1, wherein X comprises at least two tertiary amines.

11. The ionizable amphiphilic Janus dendrimer of claim 1, wherein each occurrence of X is independently selected from the group consisting of: 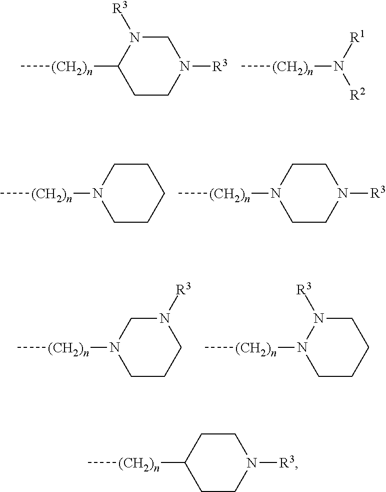
and any combination thereof,
wherein dashed lines indicate the connection to A;
each occurrence of R¹ and R² is independently selected from the group consisting of hydrogen, deuterium, alkyl, aryl, cycloalkyl, amine, heterocycloalkyl, carbonyl, and any combinations thereof, wherein R¹ and R² may together form a ring;
each occurrence of R³ is independently selected from the group consisting of hydrogen, (CH₂)ₙ, (CH₂)ₙ—OH, and any combination thereof, and
each occurrence of n is independently an integer from 1 to 20.

12. The ionizable amphiphilic Janus dendrimer of claim 1, wherein each occurrence of X is independently selected from the group consisting of: 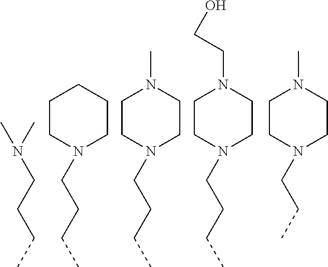
and any combination thereof.

13. The ionizable amphiphilic Janus dendrimer of claim 1, wherein each occurrence of Y is independently selected from the group consisting of: 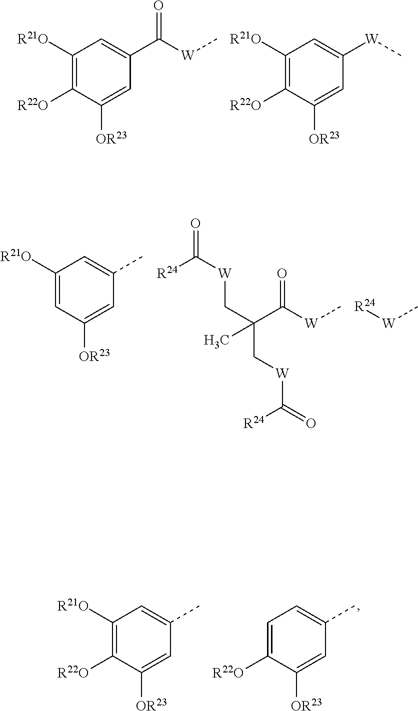
and any combination thereof;
wherein dashed lines indicate the connection to A;
each occurrence of W is independently selected from the group consisting of C═O, C(R^{W})(R^{W}), NR^{W}, O, and S;
wherein each occurrence of R^{W} is independently selected from the group consisting of hydrogen, halide, hydroxy, alkyl, alkyl halide, aryl, aryl halide, alkoxy, alkoxy halide, and any combination thereof, and
each occurrence of R²¹, R²², R²³, and R²⁴ independently comprises a linear or branched C₁-C₅₀-alkyl.

14. The ionizable amphiphilic Janus dendrimer of claim 13, wherein each occurrence of R²¹, R²², R²³, and R²⁴ independently comprises C₁-C₃₀-alkyl.

15. The ionizable amphiphilic Janus dendrimer of claim 13, wherein each occurrence of Y is independently selected from the group consisting of: 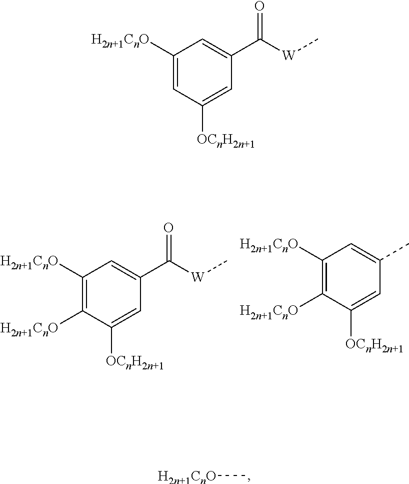
and any combination thereof;
wherein each occurrence of W is independently selected from the group consisting of C═O, C(R^{W})(R^{W}), NR^{W}, O, and S;
wherein each occurrence of R^{W} is independently selected from the group consisting of hydrogen, halide, hydroxy, alkyl, alkyl halide, aryl, aryl halide, alkoxy, alkoxy halide, and any combination thereof; and
n is an integer from 1 to 30.

16. The ionizable amphiphilic Janus dendrimer of claim 1, wherein each occurrence of Y is independently selected from the group consisting of 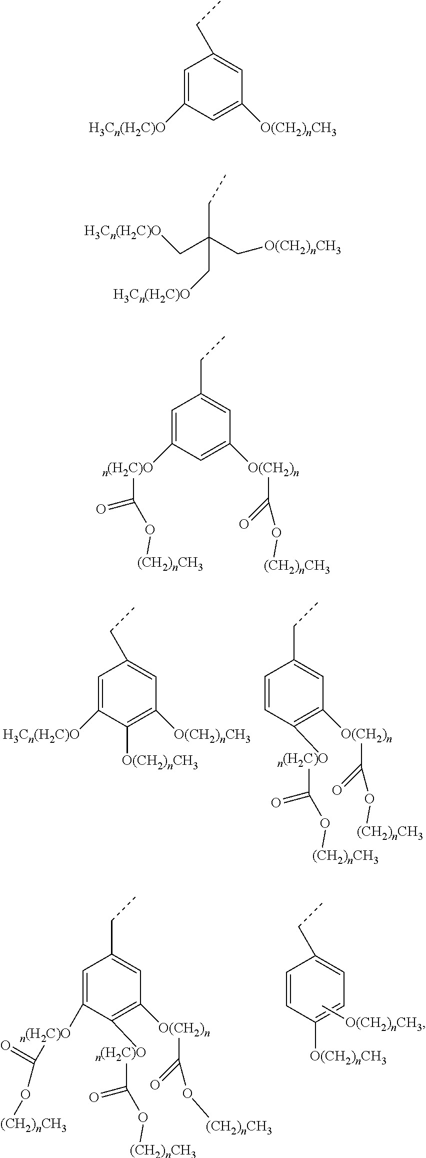
and any combination thereof;
wherein dashed lines indicate the connection to A;
each occurrence of n is independently an integer from 1 to 20; and
each occurrence of —(CH₂)ₙCH₃, —O(CH₂)ₙ—, and —O(CH₂)ₙCH₃ is independently linear or branched.

17. The ionizable amphiphilic Janus dendrimer of claim 1, wherein the ionizable amphiphilic Janus dendrimer comprises a first Y and a second Y,
wherein the first Y comprises an alkyl chain having an even number of carbon atoms, and the second Y comprises an alkyl chain having an odd number of carbon atoms.

18. The ionizable amphiphilic Janus dendrimer of claim 17, wherein the ratio between the carbon atoms in the first Y and the carbon atoms in the second Y is greater than or equal to 3 and less than 7.

19. The ionizable amphiphilic Janus dendrimer of claim 1, wherein u is 1 or 2;
each occurrence of Z is independently selected from the group consisting of: 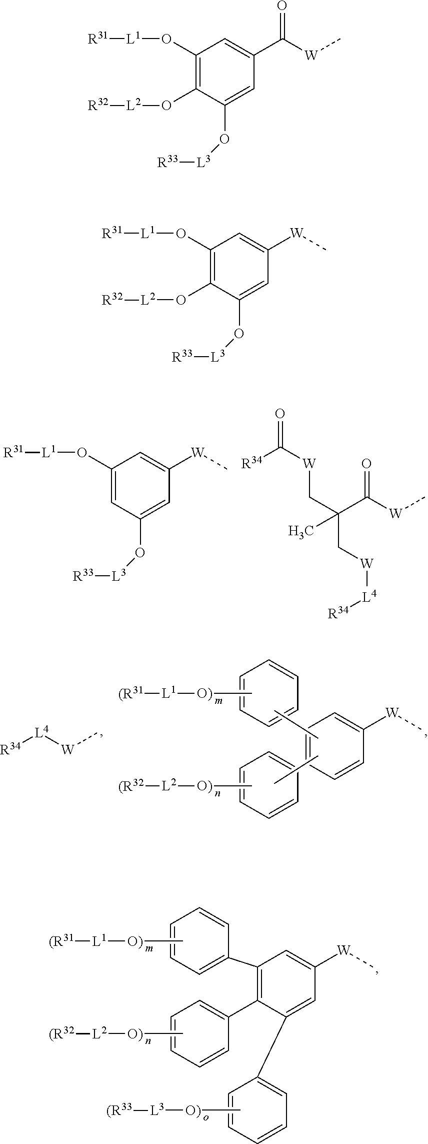
and any combination thereof;
wherein dashed lines indicate the connection to A;
each occurrence of L¹, L², L³, and L⁴ is independently a covalent bond or a divalent linking group selected from the group consisting of alkylene, cycloalkylene, heteroalkylene, heterocycloalkylene, alkenylene, alkynylene, arylene, heteroarylene, silyl, amine, amide, ester, ether, carbonyl, carbamate, thioether, thioester, disulfide, hydrazine, urea, thiourea, phosphate, poly(alkyl ether), heteroatom, and any combinations thereof;
each occurrence of R³¹, R³², R³³, and R³⁴ is independently selected from the group consisting of hydrogen, deuterium, alkyl, aryl, heteroaryl, cycloalkyl, alkoxy, phenoxy, amine, heterocycloalkyl, carbonyl, and any combinations thereof; and
each occurrence of m, n, o is independently an integer from 1 to 5.

20. The ionizable amphiphilic Janus dendrimer of claim 19, wherein each occurrence of Z is independently selected from the group consisting of: 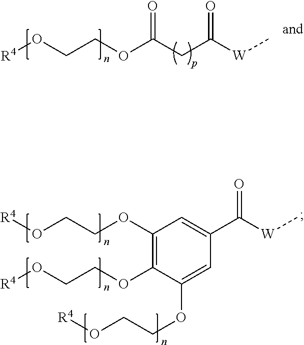
wherein each occurrence of n is independently an integer from 1 to 100;
each occurrence of p is independently an integer from 1 to 10; and
each occurrence of R⁴ is independently selected from the group consisting of hydrogen, deuterium, alkyl, aryl, and any combinations thereof.

21. The ionizable amphiphilic Janus dendrimer of claim 1, wherein the ionizable amphiphilic Janus dendrimer comprises a homochiral, racemic, or achiral branding points.

22. The ionizable amphiphilic Janus dendrimer of claim 1, wherein the ionizable amphiphilic Janus dendrimer is a homochiral ionizable amphiphilic Janus dendrimer, racemic ionizable amphiphilic Janus dendrimer, or achiral ionizable amphiphilic Janus dendrimer.

23. The ionizable amphiphilic Janus dendrimer of claim 1, wherein the ionizable amphiphilic Janus dendrimer is an ionizable amphiphilic Janus dendrimer having a structure selected from the group consisting of at least one structure of FIG. 12, at least one structure of FIG. 13, at least one structure of FIG. 14, at least one structure of FIG. 47, at least one structure of FIG. 60, at least one structure of FIG. 80, and any combination thereof.

24. The ionizable amphiphilic Janus dendrimer of claim 1, wherein the ionizable amphiphilic Janus dendrimer is an ionizable amphiphilic Janus dendrimer having a structure selected from the group consisting of

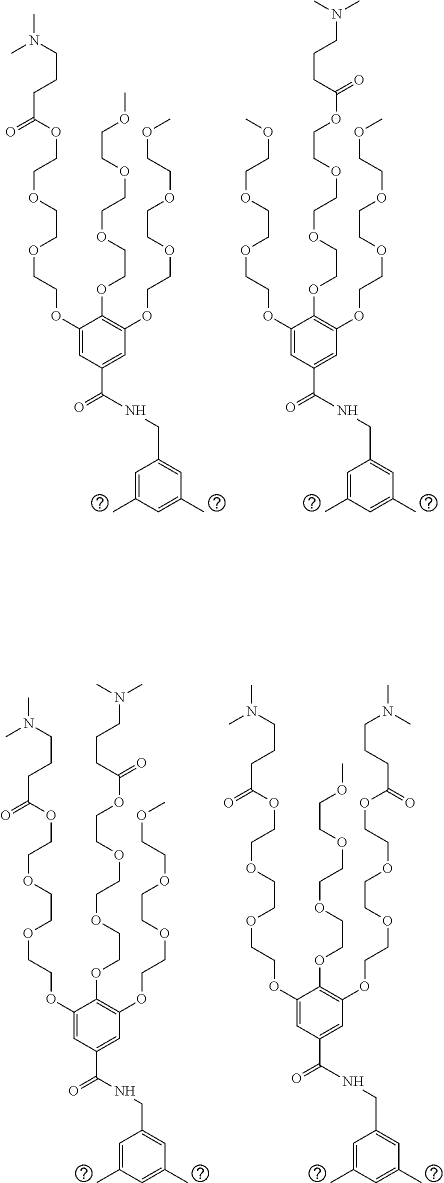 [SMILES: COCCOCCOCCOc1cc(C(=O)NCc2cc(C)cc(C)c2)cc(OCCOCCOCCOC(=O)CCCN(C)C)c1OCCOCCOCCOC ; COCCOCCOCCOc1cc(C(=O)NCc2cc(C)cc(C)c2)cc(OCCOCCOCCOC)c1OCCOCCOCCOC(=O)CCCN(C)C ; COCCOCCOCCOc1cc(C(=O)NCc2cc(C)cc(C)c2)cc(OCCOCCOCCOC(=O)CCCN(C)C)c1OCCOCCOCCOC(=O)CCCN(C)C ; COCCOCCOCCOc1c(OCCOCCOCCOC(=O)CCCN(C)C)cc(C(=O)NCc2cc(C)cc(C)c2)cc1OCCOCCOCCOC(=O)CCCN(C)C]

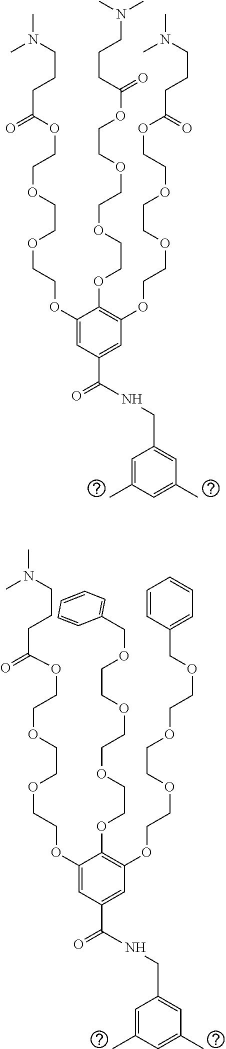 [SMILES: Cc1cc(C)cc(CNC(=O)c2cc(OCCOCCOCCOC(=O)CCCN(C)C)c(OCCOCCOCCOC(=O)CCCN(C)C)c(OCCOCCOCCOC(=O)CCCN(C)C)c2)c1 ; Cc1cc(C)cc(CNC(=O)c2cc(OCCOCCOCCOCc3ccccc3)c(OCCOCCOCCOCc3ccccc3)c(OCCOCCOCCOC(=O)CCCN(C)C)c2)c1]

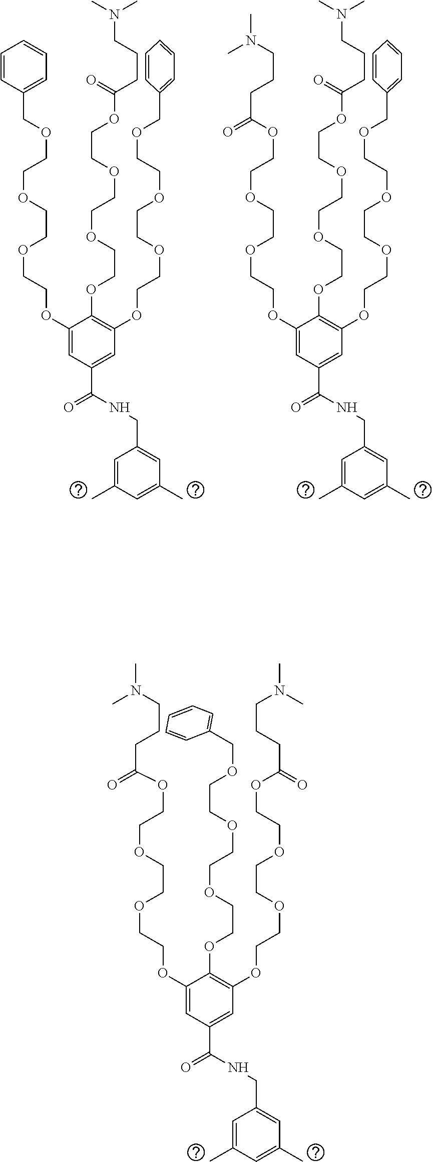 [SMILES: Cc1cc(C)cc(CNC(=O)c2cc(OCCOCCOCCOCc3ccccc3)c(OCCOCCOCCOC(=O)CCCN(C)C)c(OCCOCCOCCOCc3ccccc3)c2)c1 ; Cc1cc(C)cc(CNC(=O)c2cc(OCCOCCOCCOCc3ccccc3)c(OCCOCCOCCOC(=O)CCCN(C)C)c(OCCOCCOCCOC(=O)CCCN(C)C)c2)c1 ; Cc1cc(C)cc(CNC(=O)c2cc(OCCOCCOCCOC(=O)CCCN(C)C)c(OCCOCCOCCOCc3ccccc3)c(OCCOCCOCCOC(=O)CCCN(C)C)c2)c1]

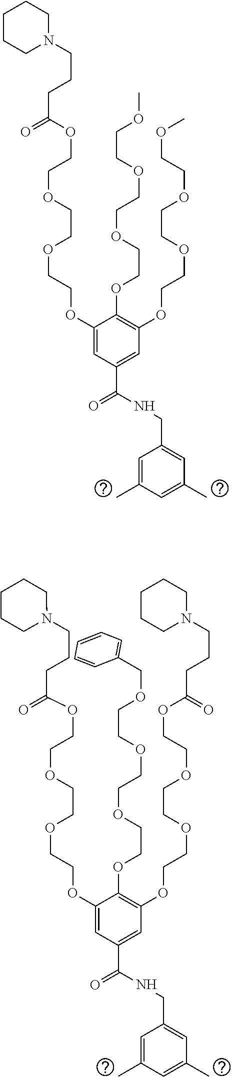 [SMILES: COCCOCCOCCOc1cc(C(=O)NCc2cc(C)cc(C)c2)cc(OCCOCCOCCOC(=O)CCCN2CCCCC2)c1OCCOCCOCCOC ; Cc1cc(C)cc(CNC(=O)c2cc(OCCOCCOCCOC(=O)CCCN3CCCCC3)c(OCCOCCOCCOCc3ccccc3)c(OCCOCCOCCOC(=O)CCCN3CCCCC3)c2)c1]

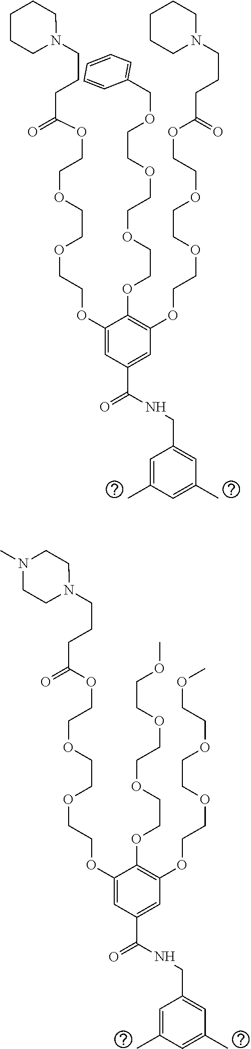

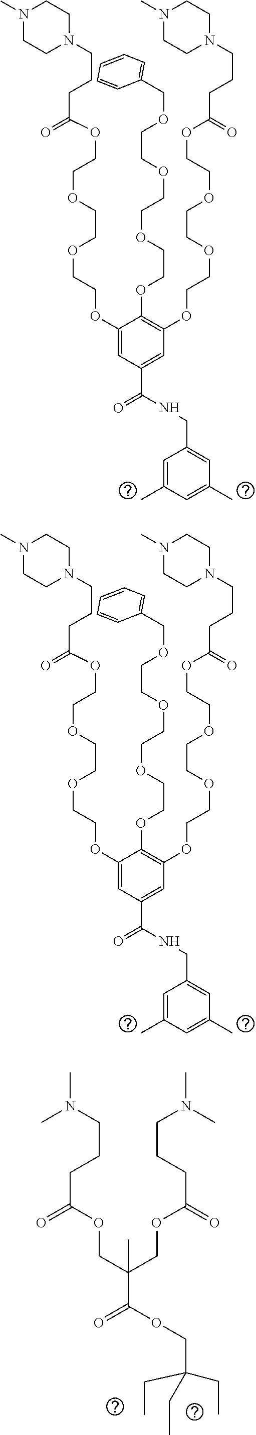

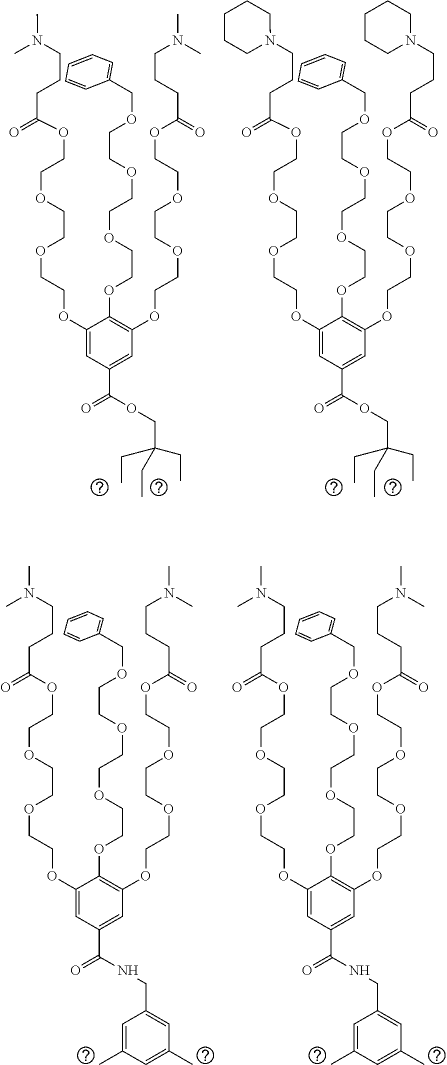

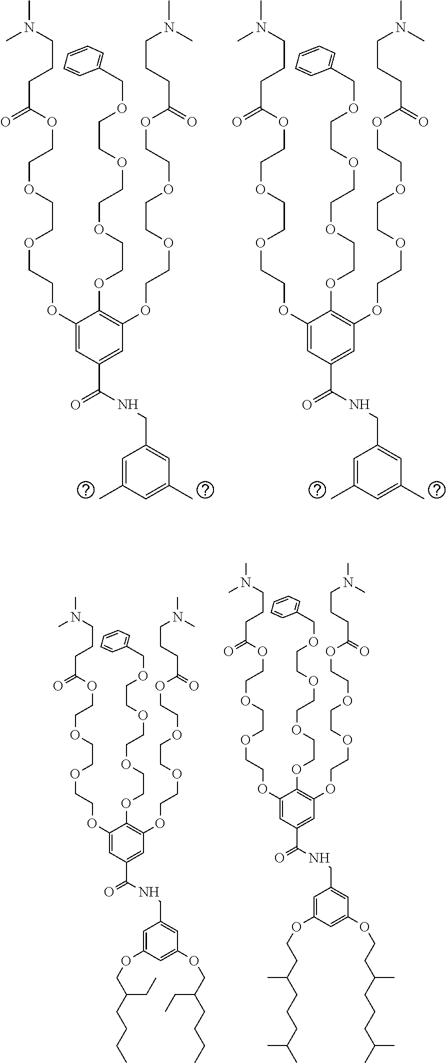 [SMILES: Cc1cc(C)cc(CNC(=O)c2cc(OCCOCCOCCOC(=O)CCCN(C)C)c(OCCOCCOCCOCc3ccccc3)c(OCCOCCOCCOC(=O)CCCN(C)C)c2)c1 ; Cc1cc(C)cc(CNC(=O)c2cc(OCCOCCOCCOC(=O)CCCN(C)C)c(OCCOCCOCCOCc3ccccc3)c(OCCOCCOCCOC(=O)CCCN(C)C)c2)c1 ; CCCCC(CC)COc1cc(CNC(=O)c2cc(OCCOCCOCCOC(=O)CCCN(C)C)c(OCCOCCOCCOCc3ccccc3)c(OCCOCCOCCOC(=O)CCCN(C)C)c2)cc(OCC(CC)CCCC)c1 ; CC(C)CCCC(C)CCOc1cc(CNC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCC(C)CCCC(C)C)c1]

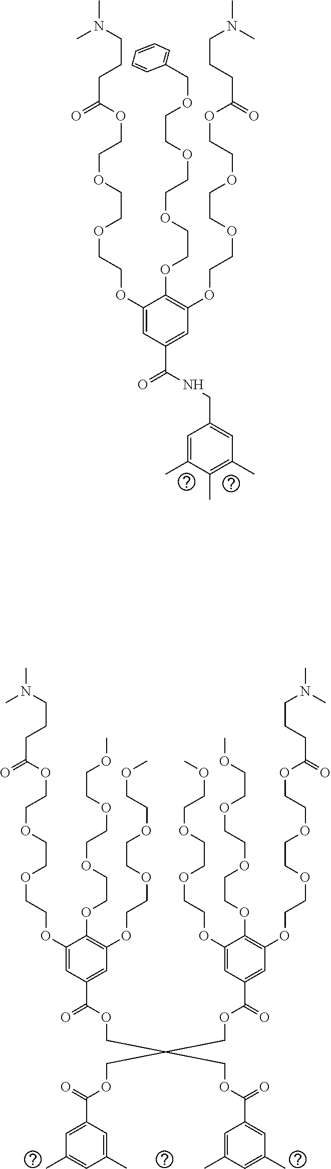 [SMILES: Cc1cc(CNC(=O)c2cc(OCCOCCOCCOC(=O)CCCN(C)C)c(OCCOCCOCCOCc3ccccc3)c(OCCOCCOCCOC(=O)CCCN(C)C)c2)cc(C)c1C ; COCCOCCOCCOc1cc(C(=O)OCC(COC(=O)c2cc(C)cc(C)c2)(COC(=O)c2cc(C)cc(C)c2)COC(=O)c2cc(OCCOCCOCCOC)c(OCCOCCOCCOC)c(OCCOCCOCCOC(=O)CCCN(C)C)c2)cc(OCCOCCOCCOC(=O)CCCN(C)C)c1OCCOCCOCCOC]

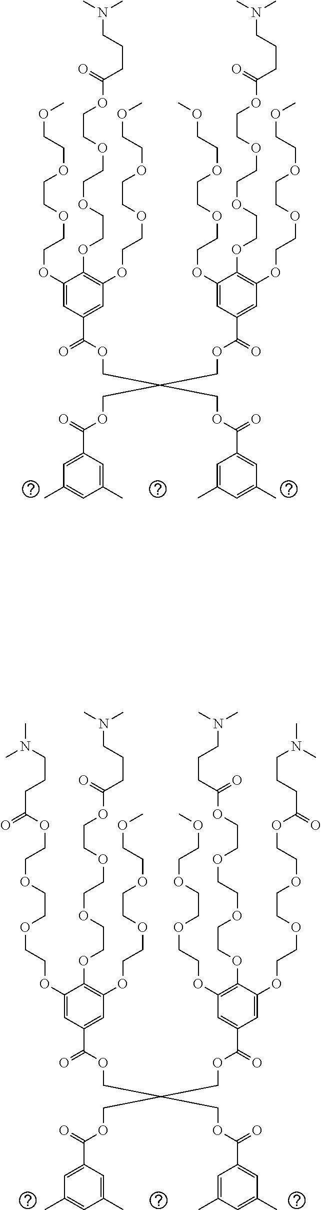 [SMILES: COCCOCCOCCOc1cc(C(=O)OCC(COC(=O)c2cc(C)cc(C)c2)(COC(=O)c2cc(C)cc(C)c2)COC(=O)c2cc(OCCOCCOCCOC)c(OCCOCCOCCOC(=O)CCCN(C)C)c(OCCOCCOCCOC)c2)cc(OCCOCCOCCOC)c1OCCOCCOCCOC(=O)CCCN(C)C ; COCCOCCOCCOc1cc(C(=O)OCC(COC(=O)c2cc(C)cc(C)c2)(COC(=O)c2cc(C)cc(C)c2)COC(=O)c2cc(OCCOCCOCCOC)c(OCCOCCOCCOC(=O)CCCN(C)C)c(OCCOCCOCCOC(=O)CCCN(C)C)c2)cc(OCCOCCOCCOC(=O)CCCN(C)C)c1OCCOCCOCCOC(=O)CCCN(C)C]

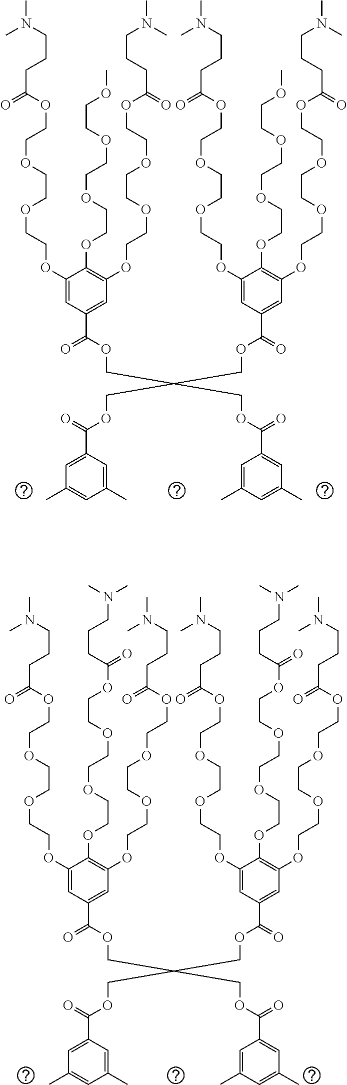 [SMILES: COCCOCCOCCOc1c(OCCOCCOCCOC(=O)CCCN(C)C)cc(C(=O)OCC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOC)c(OCCOCCOCCOC(=O)CCCN(C)C)c2)cc1OCCOCCOCCOC(=O)CCCN(C)C ; Cc1cc(C)cc(C(=O)OCC(COC(=O)c2cc(C)cc(C)c2)(COC(=O)c2cc(OCCOCCOCCOC(=O)CCCN(C)C)c(OCCOCCOCCOC(=O)CCCN(C)C)c(OCCOCCOCCOC(=O)CCCN(C)C)c2)COC(=O)c2cc(OCCOCCOCCOC(=O)CCCN(C)C)c(OCCOCCOCCOC(=O)CCCN(C)C)c(OCCOCCOCCOC(=O)CCCN(C)C)c2)c1]

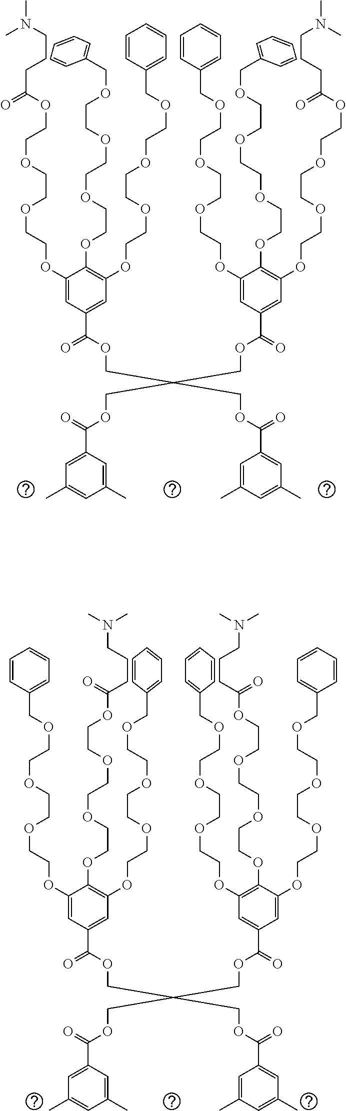 [SMILES: Cc1cc(C)cc(C(=O)OCC(COC(=O)c2cc(C)cc(C)c2)(COC(=O)c2cc(OCCOCCOCCOCc3ccccc3)c(OCCOCCOCCOCc3ccccc3)c(OCCOCCOCCOC(=O)CCCN(C)C)c2)COC(=O)c2cc(OCCOCCOCCOCc3ccccc3)c(OCCOCCOCCOCc3ccccc3)c(OCCOCCOCCOC(=O)CCCN(C)C)c2)c1 ; Cc1cc(C)cc(C(=O)OCC(COC(=O)c2cc(C)cc(C)c2)(COC(=O)c2cc(OCCOCCOCCOCc3ccccc3)c(OCCOCCOCCOC(=O)CCCN(C)C)c(OCCOCCOCCOCc3ccccc3)c2)COC(=O)c2cc(OCCOCCOCCOCc3ccccc3)c(OCCOCCOCCOC(=O)CCCN(C)C)c(OCCOCCOCCOCc3ccccc3)c2)c1]

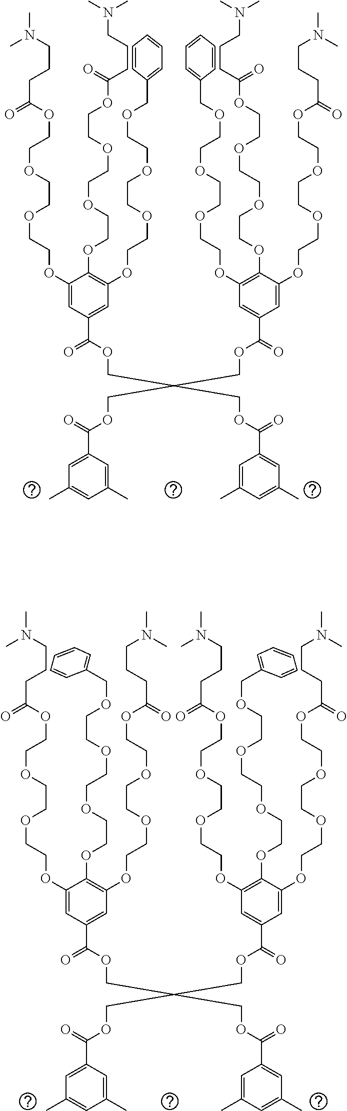 [SMILES: Cc1cc(C)cc(C(=O)OCC(COC(=O)c2cc(C)cc(C)c2)(COC(=O)c2cc(OCCOCCOCCOCc3ccccc3)c(OCCOCCOCCOC(=O)CCCN(C)C)c(OCCOCCOCCOC(=O)CCCN(C)C)c2)COC(=O)c2cc(OCCOCCOCCOCc3ccccc3)c(OCCOCCOCCOC(=O)CCCN(C)C)c(OCCOCCOCCOC(=O)CCCN(C)C)c2)c1 ; Cc1cc(C)cc(C(=O)OCC(COC(=O)c2cc(C)cc(C)c2)(COC(=O)c2cc(OCCOCCOCCOC(=O)CCCN(C)C)c(OCCOCCOCCOCc3ccccc3)c(OCCOCCOCCOC(=O)CCCN(C)C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)c1]

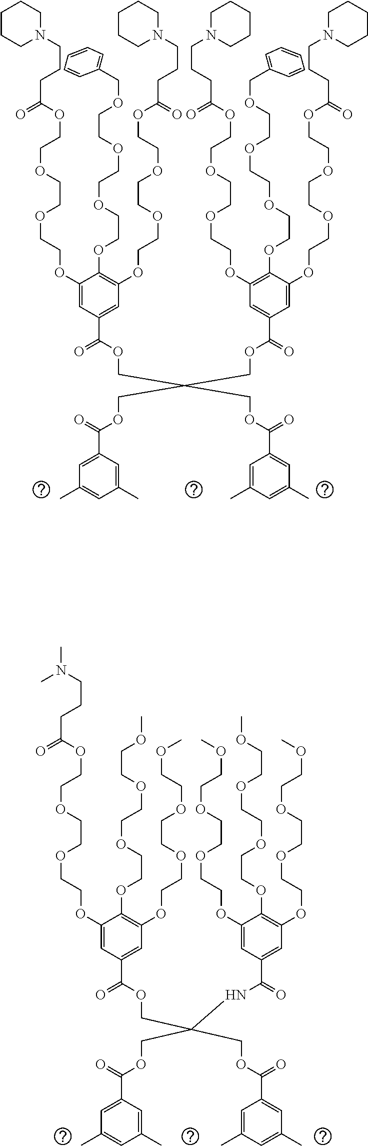 [SMILES: Cc1cc(C)cc(C(=O)OCC(COC(=O)c2cc(C)cc(C)c2)(COC(=O)c2cc(OCCOCCOCCOC(=O)CCCN3CCCCC3)c(OCCOCCOCCOCc3ccccc3)c(OCCOCCOCCOC(=O)CCCN3CCCCC3)c2)COC(=O)c2cc(OCCOCCOCCOC(=O)CCCN3CCCCC3)c(OCCOCCOCCOCc3ccccc3)c(OCCOCCOCCOC(=O)CCCN3CCCCC3)c2)c1 ; COCCOCCOCCOc1cc(C(=O)NC(COC(=O)c2cc(C)cc(C)c2)(COC(=O)c2cc(C)cc(C)c2)COC(=O)c2cc(OCCOCCOCCOC)c(OCCOCCOCCOC)c(OCCOCCOCCOC(=O)CCCN(C)C)c2)cc(OCCOCCOCCOC)c1OCCOCCOCCOC]

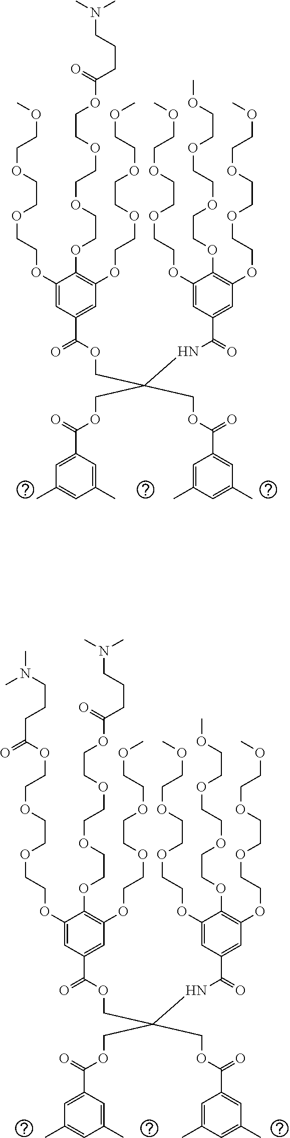 [SMILES: COCCOCCOCCOc1cc(C(=O)NC(COC(=O)c2cc(C)cc(C)c2)(COC(=O)c2cc(C)cc(C)c2)COC(=O)c2cc(OCCOCCOCCOC)c(OCCOCCOCCOC(=O)CCCN(C)C)c(OCCOCCOCCOC)c2)cc(OCCOCCOCCOC)c1OCCOCCOCCOC ; COCCOCCOCCOc1cc(C(=O)NC(COC(=O)c2cc(C)cc(C)c2)(COC(=O)c2cc(C)cc(C)c2)COC(=O)c2cc(OCCOCCOCCOC)c(OCCOCCOCCOC(=O)CCCN(C)C)c(OCCOCCOCCOC(=O)CCCN(C)C)c2)cc(OCCOCCOCCOC)c1OCCOCCOCCOC]

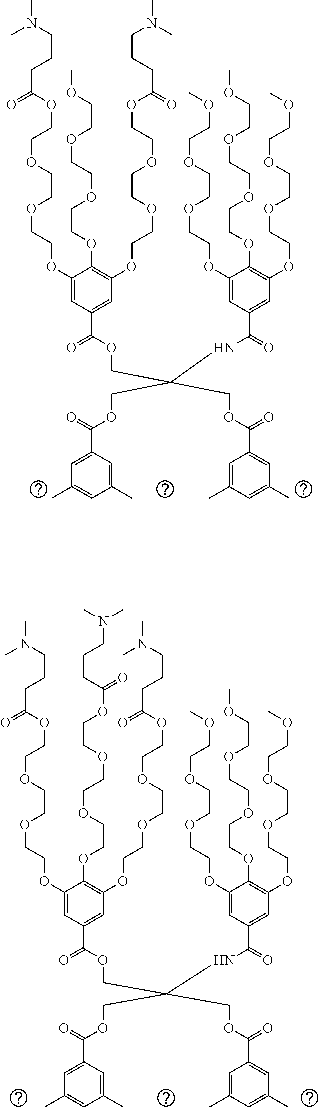 [SMILES: COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOC)c(OCCOCCOCCOC(=O)CCCN(C)C)c2)cc(OCCOCCOCCOC)c1OCCOCCOCCOC ; COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOC(=O)CCCN(C)C)c(OCCOCCOCCOC(=O)CCCN(C)C)c2)cc(OCCOCCOCCOC)c1OCCOCCOCCOC]

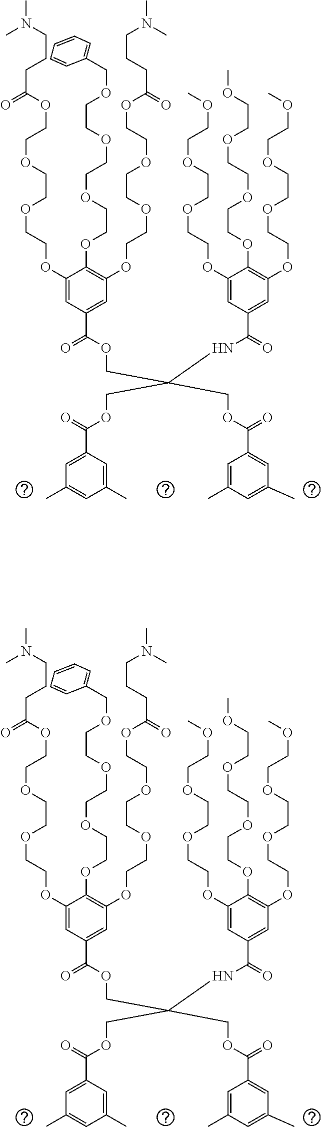 [SMILES: COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCOCCOCCOC)c1OCCOCCOCCOC ; COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCOCCOCCOC)c1OCCOCCOCCOC]

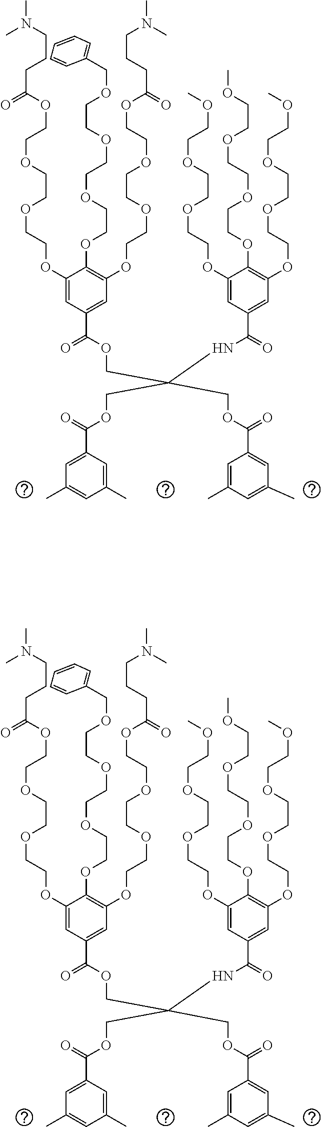 [SMILES: COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCOCCOCCOC)c1OCCOCCOCCOC ; COCCOCCOCCOc1cc(C(=O)NC(COC(=O)c2cc(C)cc(C)c2)(COC(=O)c2cc(C)cc(C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCOCCOCCOC)c1OCCOCCOCCOC]

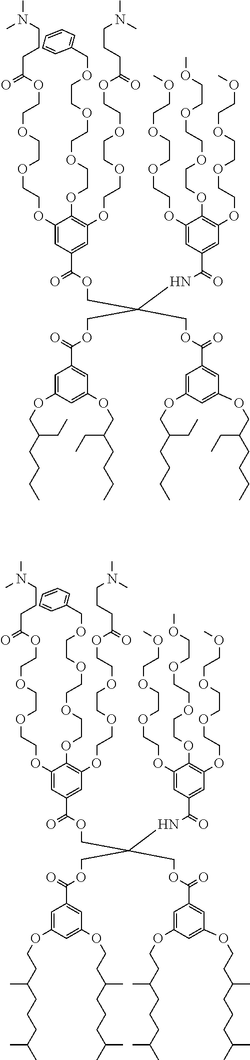 [SMILES: CCCCC(CC)COc1cc(OCC(CC)CCCC)cc(C(=O)OCC(COC(=O)c2cc(OCC(CC)CCCC)cc(OCC(CC)CCCC)c2)(COC(=O)c2cc(OCCOCCOCCOC(=O)CCCN(C)C)c(OCCOCCOCCOCc3ccccc3)c(OCCOCCOCCOC(=O)CCCN(C)C)c2)NC(=O)c2cc(OCCOCCOCCOC)c(OCCOCCOCCOC)c(OCCOCCOCCOC)c2)c1 ; COCCOCCOCCOc1cc(C(=O)NC(COC(=O)c2cc(OCCC(C)CCCC(C)C)cc(OCCC(C)CCCC(C)C)c2)(COC(=O)c2cc(OCCC(C)CCCC(C)C)cc(OCCC(C)CCCC(C)C)c2)COC(=O)c2cc(OCCOCCOCCOC(=O)CCCN(C)C)c(OCCOCCOCCOCc3ccccc3)c(OCCOCCOCCOC(=O)CCCN(C)C)c2)cc(OCCOCCOCCOC)c1OCCOCCOCCOC]

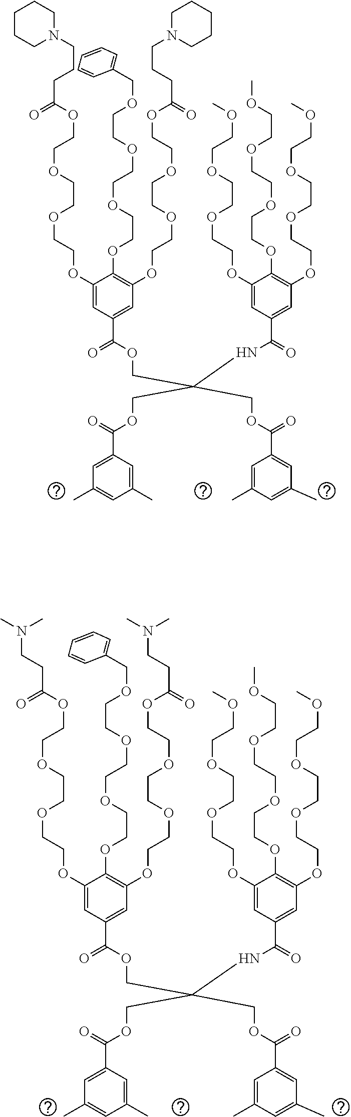

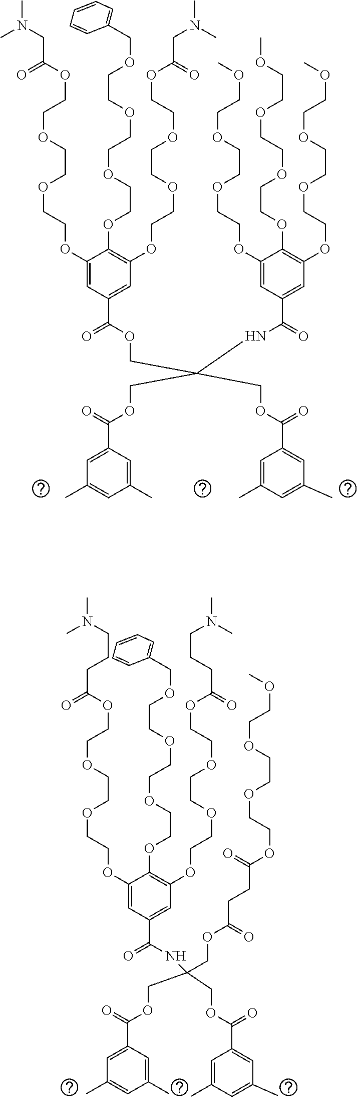

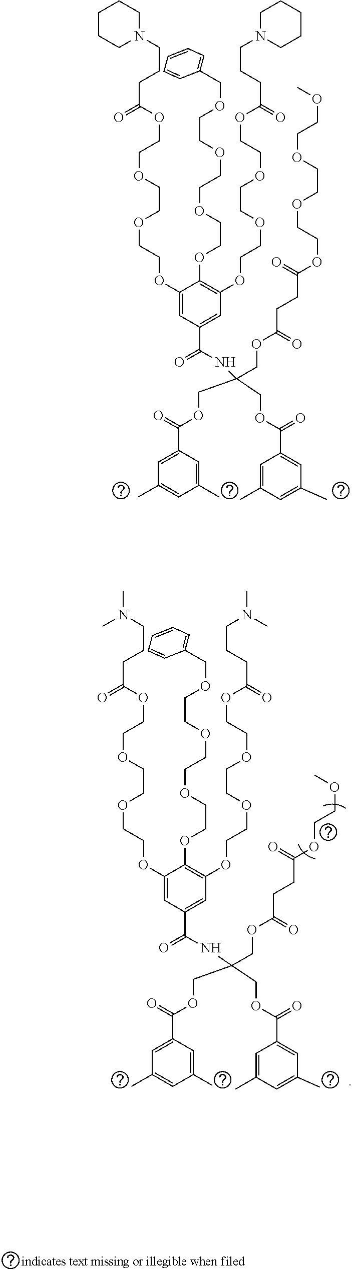

25. A nanoparticle comprising at least one ionizable amphiphilic Janus dendrimer of claim 1.

26. The nanoparticle of claim 25, wherein the nanoparticle comprises a first ionizable amphiphilic Janus dendrimer and a second ionizable amphiphilic Janus dendrimer,
wherein the first ionizable amphiphilic Janus dendrimer has a different structure than the second ionizable amphiphilic Janus dendrimer.

27. The nanoparticle of claim 25, wherein the nanoparticle comprises a homochiral ionizable amphiphilic Janus dendrimer, achiral ionizable amphiphilic Janus dendrimer, or any combination thereof.

28. The nanoparticle of claim 27, wherein the nanoparticle is a unilamellar nanoparticle or an onion multilamellar nanoparticle.

29. The nanoparticle of claim 25, wherein the nanoparticle comprises a racemic ionizable amphiphilic Janus dendrimer.

30. The nanoparticle of claim 29, wherein the nanoparticle is a multilamellar nanoparticle.

31. The nanoparticle of claim 25, wherein the nanoparticle further comprises at least one agent.

32. The nanoparticle of claim 31, wherein the at least one agent comprises a diagnostic agent, detectable agent, therapeutic agent, nucleic acid molecule, or any combination thereof.

33. The nanoparticle of claim 31, wherein the at least one agent is selected from the group consisting of an mRNA, siRNA, microRNA, CRISPR-Cas9, sgRNA, small molecule, protein, antibody, peptide, protein, and any combination thereof.

34. The nanoparticle of claim 31, wherein the at least one agent comprises a nucleic acid molecule.

35. The nanoparticle of claim 34, wherein the nucleic acid molecule is a DNA molecule or an RNA molecule.

36. The nanoparticle of claim 34, wherein the nucleic acid molecule is selected from the group consisting of cDNA, cRNA, CirRNA, mRNA, miRNA, siRNA, sgRNA, modified RNA, tRNA, antagomir, antisense molecule, targeted nucleic acid, and any combination thereof.

37. The nanoparticle of claim 34, wherein the nucleic acid molecule encodes at least one selected from the group consisting of an antigen, antibody, gene editing molecule, chimeric antigen receptor (CAR), and any combination thereof.

38. A composition comprising at least one selected from the group consisting of at least one ionizable amphiphilic Janus dendrimer of claim 1, at least one nanoparticle comprising thereof, and any combination thereof.

39. The composition of claim 38, wherein the composition further comprises an adjuvant.

40. The composition of claim 38, wherein the composition is a pharmaceutical composition.

41. The composition of claim 38, wherein the composition is a vaccine.

42. A method of delivering an agent to a subject in need thereof, wherein the method comprises administering at least one nanoparticle of claim 31 or a composition comprising the same to the subject.

43. The method of claim 42, wherein the composition further comprises an adjuvant.

44. The method of claim 42, wherein the agent is encapsulated within the nanoparticle.

45. The method of claim 42, wherein the method treats or prevents at least one condition selected from the group consisting of a viral infection, bacterial infection, fungal infection, parasitic infection, cancer, disease or disorder associated with cancer, autoimmune disease or disorder, and any combination thereof.

46. The method of claim 42, wherein the agent is a composition for protein replacement therapy.

47. The method of claim 42, wherein the agent is a composition for gene editing.

48. The method of claim 42, wherein the agent is a vaccine.

49. The method of claim 42, wherein the method further comprises delivering the agent to the liver of the subject.

50. The method of claim 42, wherein the method further comprises delivering the agent to the spleen of the subject.

51. The method of claim 42, wherein the method further comprises delivering the agent to the lungs of the subject.

52. The method of claim 42, wherein the agent comprises at least one selected from the group consisting of cDNA, cRNA, CirRNA, mRNA, miRNA, siRNA, sgRNA, modified RNA, tRNA, antagomir, antisense molecule, targeted nucleic acid, and any combination thereof.

53. The method of claim 52, wherein the modified RNA is a nucleoside-modified RNA.

54. The method of claim 53, wherein the nucleoside-modified RNA comprises pseudouridine.

55. The method of claim 53, wherein the nucleoside-modified RNA comprises pseudouridine plus 5-methyl-cytosine.

56. The method of claim 53, wherein the nucleoside-modified RNA comprises 5-methyl-uridine.

57. The method of claim 53, wherein the nucleoside-modified RNA comprises 1-methyl-pseudouridine.

58. A method of preventing or treating a disease or disorder in a subject in need thereof, wherein the method comprises administering at least one nanoparticle of claim 25 or a composition comprising the same to the subject.

59. The method of claim 58, wherein the disease or disorder is selected from the group consisting of a viral infection, bacterial infection, fungal infection, parasitic infection, cancer, disease or disorder associated with cancer, autoimmune disease or disorder, and any combination thereof.

60. A method of inducing an immune response in a subject in need thereof, wherein the method comprises administering at least one nanoparticle of claim 25 or a composition comprising the same to the subject.
